(19) United States
(12) Patent Application Publication
(10) Pub. No.: US 2026/0103736 A1
(43) Pub. Date: Apr. 16, 2026

(54) Methods Of Improving Production Of Morphinan Alkaloids And Derivatives

(71) Applicant: Antheia, Inc., Menlo Park, CA (US)

(72) Inventors: James T. PAYNE, Menlo Park, CA (US); Amy M. KOZINA, Menlo Park, CA (US); Rodrigo A. ESTRADA, Menlo Park, CA (US); Kristy M. HAWKINS, Menlo Park, CA (US); Christina D. SMOLKE, Menlo Park, CA (US)

(21) Appl. No.: 19/116,895

(22) PCT Filed: Sep. 29, 2023
(86) PCT No.: PCT/US2023/075653

§ 371 Date: Mar. 28, 2025

Related U.S. Application Data
(60) Provisional application No. 63/377,672, filed on Sep. 29, 2022.

(51) Int. Cl. C12P 17/18; C12N 1/16; C12N 9/02; C12N 15/52; C12N 15/81; C12R 1/645

(52) U.S. Cl. CPC C12P 17/18; C12N 1/16; C12N 9/0008; C12N 9/0042; C12N 9/0071; C12N 15/52; C12N 15/81; C12Y 102/01046; C12Y 106/02004; C12Y 114/14; C12R 2001/645

(57) ABSTRACT

Disclosed herein are methods that may be used for the synthesis of benzylisoquinoline alkaloids (“BIAs”) such as alkaloid morphinan. The methods disclosed can be used to produce thebaine, oripavine, codeine, morphine, oxycodone, hydrocodone, oxymorphone, hydromorphone, naltrexone, naloxone, hydroxycodeinone, neopinone, and/or buprenorphine.

### CROSS-REFERENCE TO RELATED APPLICATION

This application claims the benefit of priority to U.S. Provisional Patent Application No. 63/377,672 filed Sep. 29, 2022, the entire contents of which are incorporated by reference herein and for all purposes.

### BACKGROUND

The existing manufacturing methods for BIAs, including morphinan alkaloids and their derivatives, suffer from low yields and/or are expensive. Some of the known methodologies for the manufacture of BIAs exist in the production of undesirable quantities of morphinan alkaloid by-products (see e.g., Rinner, U., and Hudlicky, J., 2012, Top. Cur. Chem. 209: 33-66). No methods exist to commercially biosynthetically manufacture BIAs, including morphinan alkaloids and their derivatives. Therefore, there is a need for improved methods for the synthesis of BIAs including morphinan alkaloids and their derivatives.

### SUMMARY

The present disclosure provides methods for the production of diverse benzylisoquinoline alkaloids (BIAs) in engineered host cells. In certain embodiments, the engineered host cell is a non-plant cell. The present disclosure further provides compositions of diverse alkaloids produced in engineered host cells. Additionally, the present disclosure provides methods for the expression of one or more enzymes providing C-14-hydroxylase activity in engineered host cells. In certain embodiments, the one or more enzymes providing C-14-hydroxylase activity is a cytochrome P450 protein (“P450”). In some embodiments, the one or more enzymes providing C-14-hydroxylase activity is heterologous to the engineered host cell. Additionally, the present disclosure provides methods for the expression of one or more engineered P450s providing C-14-hydroxylase activity in engineered host cells. In particular cases, the disclosure provides methods for increasing production of diverse alkaloid products by the use of P450s providing C-14-hydroxylase activity. In some embodiments, the disclosure provides methods for increasing production of diverse alkaloid products through the overexpression of formaldehyde dehydrogenases. In some embodiments, the disclosure provides methods for increasing production of diverse alkaloid products through the overexpression of alcohol dehydrogenases. In further embodiments, the present disclosure provides methods for increasing production of diverse alkaloid products through preventing expression of glutamine amidotransferases. In some embodiments, the disclosure provides methods for increasing production of diverse alkaloid products through the conversion of a precursor morphinan alkaloid with a free hydrogen at carbon C-14 into a product morphinan alkaloid with a hydroxyl group at carbon C-14 via one or more enzymes providing C-14 hydroxylase activity in an engineered host cell. In further embodiments, the precursor morphinan alkaloid with a hydrogen at carbon C-14 is produced in the engineered cell via a heterologous biosynthetic pathway comprising a plurality of enzymes and starting with simple starting materials such as sugar and/or L-tyrosine. In some embodiments, the disclosure provides methods for increasing production of diverse alkaloid products through the conversion of a precursor morphinan alkaloid with a free hydrogen at carbon C-14 into a product morphinan alkaloid with a hydroxyl group at carbon C-14 via one or more enzymes providing C-14 hydroxylase activity in an engineered host cell. In certain embodiments, the one or more enzymes providing C-14-hydroxylase activity is heterologous to the engineered host cell. In other embodiments, the present disclosure provides methods for increasing production of diverse alkaloid products through the conversion of codeine to 14-hydroxycodeine via an enzyme providing C-14-hydroxylase activity. In further embodiments, the present disclosure provides methods for increasing production of diverse alkaloid products through the conversion of codeinone to 14-hydroxycodeinone via an enzyme providing C-14 hydroxylase activity. In further embodiments, the present disclosure provides methods for increasing production of diverse alkaloid products through the conversion of codeine to 14-hydroxycodeine via an enzyme providing C-14 hydroxylase activity. In some cases, the method further comprises engineering the host cell to comprise a plurality of heterologous enzymes to produce the diverse benzylisoquinoline alkaloid products from simple starting materials such as sugar and/or L-tyrosine. In some examples, an engineered host cell (e.g., non-plant cell) comprises a plurality of coding sequences each encoding an enzyme that is selected from the group of enzymes listed in Table 17. In some examples, the heterologous coding sequences may be operably connected. Heterologous coding sequences that are operably connected may be within the same pathway of producing a particular benzylisoquinoline alkaloid product via P450s, including one or more enzymes providing C-14 hydroxylase activity. In some cases, the method further comprises engineering the host cell with a plurality of heterologous enzymes to increase the production of BIA precursors, including L-tyrosine and 4-HPAA. In some examples, an engineered host cell comprises a plurality of coding sequences each encoding an enzyme that is selected from the group of enzymes listed in Table 17. In some examples, an engineered host cell further comprises inactivating mutations in selected enzymes that result in reduced production of byproducts. In some examples, an engineered host cell further comprises heterologous expression or overexpression of selected enzymes that result in reduced production of byproducts. In some examples, the byproducts comprise formaldehyde, tyrosol, phenylethanol, or methionol. In some examples, an engineered host cell further comprises inactivating mutations in selected enzymes that result in increased production of diverse benzylisoquinoline alkaloid products.

In some embodiments, the present disclosure provides a method of producing a benzylisoquinoline alkaloid (BIA) product in an engineered host cell, the method comprising: (a) expressing a heterologous enzyme having 14-hydroxylase activity in the engineered host cell and (b) optionally expressing a heterologous enzyme having cytochrome P450 reductase (CPR) activity in the engineered host cell, (c) contacting the heterologous enzyme with a BIA-precursor substrate that is a morphinan alkaloid with a free hydrogen at carbon C-14, wherein the heterologous enzyme hydroxylates the C-14 carbon on the BIA-precursor substrate; and (d) producing the BIA product within the host cell; wherein the engineered host cell produces more BIA product than a non-engineered host cell.

In some embodiments, the method comprises producing the product of the 14-hydroxylation of the BIA-precursor substrate described in (c) within the host cell.

In some embodiments, the method further comprises (e) producing a further BIA product downstream of the molecule described in (d) through the action of one or more additional enzymes.

In some embodiments, the engineered host cell produces at least 1.5 fold more of the BIA product than the same host cell which does not comprise enzymes a) and/or b).

In some embodiments, the engineered host cell produces at least 50% more of the BIA product than the same engineered host cell which does not comprise (enzymes a) and/or b)).

In some embodiments, the method further comprises providing a BIA-precursor substrate to the engineered host cell.

In some embodiments, the heterologous enzyme is a cytochrome P450.

In some embodiments, the enzyme is capable of converting codeine to 14-hydroxycodeine.

In some embodiments, the heterologous enzyme comprises an amino acid sequence having at least 90%, 95%, 96%, 97%, 98% or 99% sequence identity to SEQ ID NOs.: 190, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 178, 180, 182, 184, 186, 188, 192, 194, 196, 198, 200, 202, 204, 206, 208, 210, 212, 214, 216, 218, 220, 222, 224, 226, 228, 230, 232, 234, 236, 238, 240, 242, or 244.

In some embodiments, the heterologous enzyme comprises or consists of the amino acid sequence of SEQ ID NOs: 190, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 178, 180, 182, 184, 186, 188, 192, 194, 196, 198, 200, 202, 204, 206, 208, 210, 212, 214, 216, 218, 220, 222, 224, 226, 228, 230, 232, 234, 236, 238, 240, 242, or 244.

In some embodiments, the host cell is a cell that does not express enzyme a) and/or enzyme b).

In some embodiments, the BIA-precursor substrate is selected from the group consisting of codeine, codeinone, norcodeinone, hydrocodone, northebaine, oripavine, morphinone, normorphinone, hydromorphine, norhydromorphine, and norhydrocodone.

In some embodiments, the BIA-precursor substrate is codeinone, codeine, hydrocodone, or hydromorphone.

In some embodiments, the BIA product is noroxymorphone.

In some embodiments, the BIA product is 14-hydroxycodeine.

In some embodiments, the engineered host cell expresses a cytochrome P450 reductase (CPR).

In some embodiments, the CPR is heterologous to the engineered host cell.

In some embodiments, the CPR comprises an amino acid sequence having at least 90%, 95%, 96%, 97%, 98% or 99% sequence identity to SEQ ID NO: 168, 170, 172,174, 248, 250, 252, 254, 256, 258, 260, 262, 264, 266, 268, or 270.

In some embodiments, the CPR comprises or consists of SEQ ID NO: 168, 170, 172,174, 248, 250, 252, 254, 256, 258, 260, 262, 264, 266, 268, or 270.

In some embodiments, the CPR is a fungal CPR.

In some embodiments, the CPR is a plant CPR.

In some embodiments, the CPR is an animal CPR.

In some embodiments, the cytochrome P450 and the CPR are from the same genus.

Also provided herein, is a host cell that produces a BIA product, the host cell comprising a first heterologous polynucleotide that encodes a heterologous enzyme having 14-hydroxylase activity and a second heterologous polynucleotide that encodes a cytochrome P450 reductase (CPR).

In some embodiments, the enzyme having 14-hydroxylase is a cytochrome P450.

In some embodiments, the enzyme having 14-hydroxylase activity comprises an amino acid sequence having at least 90%, 95%, 96%, 97%, 98% or 99% sequence identity to 190, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 178, 180, 182, 184, 186, 188, 192, 194, 196, 198, 200, 202, 204, 206, 208, 210, 212, 214, 216, 218, 220, 222, 224, 226, 228, 230, 232, 234, 236, 238, 240, 242, or 244.

In some embodiments, the CPR is a fungal CPR.

In some embodiments, the CPR is a plant CPR.

In some embodiments, the CPR is an animal CPR.

In some embodiments, the CPR comprises an amino acid sequence having at least 90%, 95%, 96%, 97%, 98% or 99% sequence identity to SEQ ID NO. 168, 170, 172,174, 248, 250, 252, 254, 256, 258, 260, 262, 264, 266, 268, or 270.

In some embodiments, the production of the BIA product comprises hydroxylation of a C-14 carbon on a BIA-precursor substrate.

In some embodiments, the host cell is a microbial cell, a fungal cell, or a yeast cell.

In some embodiments, the host cell comprises a nucleic acid construct that comprises a promoter.

Also provided herein is a vector comprising a first polynucleotide that encodes an enzyme having 14-hydroxylase activity and a second polynucleotide that encodes a cytochrome P450 reductase (CPR).

Also provided herein, is a nucleic acid construct comprising a first polynucleotide that encodes an enzyme having 14-hydroxylase activity and a second polynucleotide that encodes a cytochrome P450 reductase (CPR).

In some embodiments, the first polynucleotide and/or the second polynucleotide is codon optimized for expression in a host cell.

In some embodiments, the enzyme having 14-hydroxylase is a cytochrome P450.

In some embodiments, the polynucleotide that encodes the enzyme having 14-hydroxylase activity comprises a nucleotide sequence selected from SEQ ID Nos. 191, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 179, 181, 183, 185, 187, 189, 193, 195, 197, 199, 201, 203, 205, 207, 209, 211, 213, 215, 217, 219, 221, 223, 225, 227, 229, 231, 233, 235, 237, 239, 241, 243, or 245.

In some embodiments, the CPR is a fungal CPR.

In some embodiments, the CPR is a plant CPR.

In some embodiments, the CPR is an animal CPR.

In some embodiments, the polynucleotide that encodes the CPR comprises a nucleotide sequence selected from SEQ ID Nos. 169, 171, 173, 175, 249, 251, 253, 255, 257, 259, 261, 263, 265, 267, 269, and 271.

In some embodiments, wherein the enzyme having 14-hydroxylase activity comprises an amino acid sequence having at least 90%, 95%, 96%, 97%, 98% or 99% sequence identity to SEQ ID NO. 190, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 178, 180, 182, 184, 186, 188, 192, 194, 196, 198, 200, 202, 204, 206, 208, 210, 212, 214, 216, 218, 220, 222, 224, 226, 228, 230, 232, 234, 236, 238, 240, 242, or 244.

In some embodiments, the CPR comprises an amino acid sequence having at least 90%, 95%, 96%, 97%, 98% or 99% sequence identity to SEQ ID NO. 168, 170, 172,174, 248, 250, 252, 254, 256, 258, 260, 262, 264, 266, 268, or 270.

Also provided herein is a host cell comprising a vector that comprises a first polynucleotide that encodes an enzyme having 14-hydroxylase activity and a second polynucleotide that encodes a cytochrome P450 reductase (CPR).

In some embodiments, the first polynucleotide and/or the second polynucleotide is codon optimized for expression in a host cell.

In some embodiments, the enzyme having 14-hydroxylase is a cytochrome P450.

In some embodiments, the polynucleotide that encodes the enzyme having 14-hydroxylase activity comprises a nucleotide sequence selected from SEQ ID Nos. 191, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 179, 181, 183, 185, 187, 189, 193, 195, 197, 199, 201, 203, 205, 207, 209, 211, 213, 215, 217, 219, 221, 223, 225, 227, 229, 231, 233, 235, 237, 239, 241, 243, or 245.

In some embodiments, the CPR is a fungal CPR.

In some embodiments, the CPR is a plant CPR.

In some embodiments, the CPR is an animal CPR.

In some embodiments, the polynucleotide that encodes the CPR comprises a nucleotide sequence selected from SEQ ID Nos. 169, 171, 173, 175, 249, 251, 253, 255, 257, 259, 261, 263, 265, 267, 269, and 271.

In some embodiments, wherein the enzyme having 14-hydroxylase activity comprises an amino acid sequence having at least 90%, 95%, 96%, 97%, 98% or 99% sequence identity to SEQ ID NO. 190, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 178, 180, 182, 184, 186, 188, 192, 194, 196, 198, 200, 202, 204, 206, 208, 210, 212, 214, 216, 218, 220, 222, 224, 226, 228, 230, 232, 234, 236, 238, 240, 242, or 244.

In some embodiments, the CPR comprises an amino acid sequence having at least 90%, 95%, 96%, 97%, 98% or 99% sequence identity to SEQ ID NO. 168, 170, 172,174, 248, 250, 252, 254, 256, 258, 260, 262, 264, 266, 268, or 270.

Also provided herein is a host cell comprising a vector that comprises a first heterologous polynucleotide sequence that encodes a heterologous enzyme having formaldehyde dehydrogenase activity and a second heterologous polynucleotide designed to repress expression of a target gene, wherein the target gene is selected from DUG2 or DUG3.

In some embodiments, the heterologous enzyme is SFA1.

In some embodiments, the host cell further comprises a third polynucleotide that encodes an enzyme having 14-hydroxylase activity and a fourth polynucleotide that encodes a cytochrome P450 reductase (CPR).

Also provided herein is an isolated polypeptide having 14-hydroxylase activity.

Also provided herein is an engineered polypeptide having 14-hydroxylase activity.

Also provided herein is an enzyme mixture comprising polypeptide or an engineered polypeptide having 14-hydroxylase activity and a polypeptide having CPR activity.

Also provided herein is an in vitro method of producing a benzylisoquinoline alkaloid (BIA) product, the method comprising contacting a BIA-precursor substrate that is a morphinan alkaloid with a free hydrogen at carbon C-14 with an enzyme mixture comprising a polypeptide having 14-hydroxylase activity and a polypeptide having CPR activity, wherein the heterologous enzyme having 14-hydroxylase activity hydroxylates the C-14 carbon on the BIA-precursor substrate, thereby producing a 14-hydroxylated BIA product.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A better understanding of the features and advantages of the disclosure will be obtained by reference to the following detailed description that sets forth illustrative embodiments, in which the principles of the disclosure are utilized, and the accompanying drawings of which:

FIG. 1 illustrates a biosynthetic scheme for conversion of glucose to 4-HPAA, dopamine, 3,4-DHPAA, and 1-benzylisoquinoline alkaloids to reticuline, in accordance with some embodiments of the disclosure.

FIG. 2 illustrates examples of tyrosine hydroxylase activities, and synthesis, recycling, and salvage pathways of tetrahydrobiopterin associated with tyrosine 3-monooxygenase activities, in accordance with some embodiments of the disclosure.

FIG. 3 illustrates a biosynthetic scheme for conversion of L-tyrosine to reticuline via norcoclaurine and norlaudanosoline, in accordance with some embodiments of the disclosure.

FIG. 4 illustrates a biosynthetic scheme for conversion of L-tyrosine to morphinan alkaloids, including natural and semi-synthetic opioids, in accordance with some embodiments of the disclosure.

FIG. 5 illustrates a biosynthetic scheme for production of natural opioids, including isomers of codeine and morphine, in accordance with some embodiments of the disclosure.

FIG. 6 illustrates a biosynthetic scheme for production of nor-opioids and nal-opioids, in accordance with some embodiments of the disclosure.

FIG. 7 illustrates a biosynthetic scheme for production of sanguinarine and related pathway metabolites, in accordance with some embodiments of the disclosure.

FIG. 8 illustrates a biosynthetic scheme for production of berberine and related pathway metabolites, in accordance with some embodiments of the disclosure.

FIG. 9 illustrates an enzyme having opioid 6-O-demethylase activity, in accordance with some embodiments of the disclosure.

FIG. 10 illustrates an enzyme having opioid 3-O-demethylase activity, in accordance with some embodiments of the disclosure.

FIG. 11 illustrates certain substrates for an enzyme having opioid 14-hydroxylase activity and resulting products, in accordance with some embodiments of the disclosure.

FIG. 12 illustrates an enzyme having opioid alcohol oxidoreductase activity, in accordance with some embodiments of the disclosure.

FIG. 13 illustrates an enzyme having opioid reductase activity, in accordance with some embodiments of the disclosure.

FIG. 14 illustrates an enzyme having opioid isomerase activity, in accordance with some embodiments of the disclosure.

FIG. 15 illustrates an enzyme having N-methyltransferase activity, in accordance with some embodiments of the disclosure.

FIG. 16 illustrates yeast platform strains for the production of reticuline from L-tyrosine, in accordance with some embodiments of the disclosure.

FIG. 17 illustrates yeast strains for the production of thebaine and hydrocodone from L-tyrosine, in accordance with some embodiments of the disclosure.

FIGS. 18A-18C illustrates the production of morphinan alkaloids from sugar and L-tyrsoine from engineered yeast strains, in accordance with some embodiments of the disclosure.

FIG. 19 illustrates an enzyme having norcoclaurine synthase activity, in accordance with some embodiments of the disclosure.

FIG. 20 depicts a phylogenetic tree of selected plant Bet v I proteins with predicted NCS activity. Represented species are Coptis japonica, Thalictrum flavum, Argemone mexicana, Sinopodophyllum hexandrum, Papaver bracteatum, Papaver somniferum, and. Cordalyis saxicola, in accordance with some embodiments of the disclosure.

FIG. 21 depicts N-terminal truncations of CjNCS (SEQ ID NO: 69) and the effect on enzymatic activity, in accordance with some embodiments of the disclosure.

FIG. 22 depicts the key residues identified in a directed evolution screen of NCS (SEQ ID NO: 70) (Table 6) mapped to the crystal structure of TfNCS (PDB: 5N8Q), in accordance with some embodiments of the disclosure.

FIG. 23 depicts the key residues for improving norcoclaurine synthase activity in the template NCS parent (SEQ ID NO: 70) and in NCS variants from Coptis japonica, Thalictrum flavum, Argemone mexicana, Sinopodophyllum hexandrum, Papaver bracteatum, Papaver somniferum, and Cordalyis Saxicola (SEQ ID NOS 69 and 75-82, respectively, in order of appearance), in accordance with some embodiments of the disclosure.

FIG. 24 depicts engineered NCS variants with enhanced norcoclaurine synthase activity, in accordance with some embodiments of the disclosure.

FIG. 25 depicts norcoclaurine synthase activity in the presence of increasing dopamine concentration, in accordance with some embodiments of the disclosure.

FIG. 26 depicts a bioprocess for thebaine, in accordance with some embodiments of the disclosure.

FIG. 27 depicts another bioprocess for thebaine, in accordance with some embodiments of the disclosure.

FIG. 28 illustrates a biosynthetic scheme for conversion of glucose to 4-HPAA, dopamine, 3,4-DHPAA, and 1-benzylisoquinoline alkaloids to reticuline, in accordance with some embodiments of the disclosure.

FIG. 29 illustrates a biosynthetic scheme for conversion of chorismate to tyrosine and phenylalanine through the arogenate intermediate, in accordance with some embodiments of the disclosure.

FIG. 30 illustrates a biosynthetic scheme for glycolysis with the phosphoketalase providing a route to acetyl-CoA, in accordance with some embodiments of the disclosure.

FIG. 31 depicts a bioprocess for thebaine, in accordance with some embodiments of the disclosure.

FIG. 32 depicts a bioprocess for thebaine, in accordance with some embodiments of the disclosure.

FIG. 33 depicts a bioprocess for thebaine, in accordance with some embodiments of the disclosure.

FIG. 34 illustrates a biosynthetic scheme for the recycling of methionine in accordance with some embodiments of the disclosure.

FIG. 35 depicts a bioprocess for thebaine, in accordance with some embodiments of the disclosure.

FIGS. 36A-36D illustrate exemplary biosynthetic schemes for producing morphanin intermediates involving removal of a methyl group via oxidation (demethylation) and resultant formaldehyde byproduct formation. FIG. 36A illustrates a biosynthetic scheme for converting thebaine to codeine showing oxidation of a morphanin intermediate methylated at position 6 producing formaldehyde as a byproduct in accordance with some embodiments of the disclosure. In some embodiments, an enzyme providing 14-hydroxylation activity subsequently acts on codeinone, codeine, and/or a downstream morphanin intermediate to add a hydroxyl group to a free hydrogen at the C14 position of codeine or the morphanin intermediate. FIG. 36B illustrates a biosynthetic scheme for converting thebaine to oripavine showing oxidation of a morphinan intermediate methylated at position 6 by 3-O-demethylase and producing formaldehyde as a byproduct in accordance with some embodiments with the disclosure. FIG. 36C illustrates a biosynthetic scheme for converting codeine to morphine using codeine O-demethylase (CODM) to oxidize a morphanin intermediate and producing formaldehyde as a byproduct in accordance with some embodiments of the disclosure. FIG. 36D illustrates a biosynthetic scheme for converting thebaine to northebaine using N-demethylase to oxidize a morphanin intermediate and producing formaldehyde as a byproduct in accordance with some embodiments of the disclosure.

FIG. 37 illustrates a bioprocess for formaldehyde detoxification utilizing the formaldehyde dehydrogenase enzyme SFA1 in accordance with some embodiments of the disclosure. In some embodiments, biosynthetic schemes for formaldehyde detoxification are provided to enhance morphanin alkaloid production.

FIGS. 38A-38H illustrate exemplary biosynthetic schemes for conversion of thebaine to noroxymorphone in accordance with some embodiments of the present disclosure.

FIGS. 39A and 39B depict charts quantifying codeine (FIG. 39A) and codeinone (FIG. 38B) production in engineered host cells in accordance with some embodiments of the present disclosure.

FIG. 40 depicts a chart quantifying 14-hydroxycodeine production by engineered host cells expressing a P450 variant and a cytochrome P450 reductase (CPR) variant in accordance with some embodiments of the present disclosure.

FIG. 41 depicts a chart quantifying 14-hydroxycodeine production by engineered host cells expressing a P450 variant and a CPR variant in accordance with some embodiments of the present disclosure.

FIG. 42 depicts a chart quantifying 14-hydroxycodeine production by engineered host cells expressing a P450 variant and a CPR variant in accordance with some embodiments of the present disclosure.

FIG. 43 depicts a chart quantifying 14-hydroxycodeine production by engineered host cells expressing a P450 variant and a CPR variant in accordance with some embodiments of the present disclosure.

FIG. 44 depicts a chart quantifying 14-hydroxycodeine production by engineered host cells expressing a P450 variant and a CPR variant in accordance with some embodiments of the present disclosure.

FIG. 45 depicts an exemplary vector for incorporating a CPR (here, 14HC_CPR 1) into a microbial strain.

FIG. 46 depicts an exemplary vector for incorporating a P450 into a microbial strain (here, 14HC_P450_5).

FIGS. 47A-47D depict charts quantifying thebaine (FIG. 47A), reticuline (FIG. 47B), salutaridine (FIG. 47C), and codeine (FIG. 47D) production by engineered de novo codeine 14-hydroxylase strains expressing a CPR (here, 14HC_CPR 1) and either a 14-hydroxylase (here, 14HC_P450_5) or an empty vector control.

FIG. 48 depicts the 14-hydroxycodeine titer (nM) produced by the expression of different CPRs in YA4997.

FIG. 49 depicts fold improvement over 14HC_P450_5 in in vivo 14-hydroxycodeine production of individual point mutations.

FIG. 50 depicts comparisons of in vivo 14-hydroxycodeine titer improvements relative to 14HC_P450_5 upon addition of E58K point mutations.

FIG. 51 depicts in vivo fold improvements over 14HC_P450_5 in 14-hydroxycodeine production of various combinations of 14HC_P450_5 point mutations.

FIG. 52 depicts in vivo fold improvements over 14HC_P450_5 in 14-hydroxycodeine production (white) and oxycodone production (gray) of engineered 14HC_P450_5 variants.

FIGS. 53A-53I show 14-hydroxycodeine titers for each mutation normalized for each amino acid position. As shown in FIG. 53A, at position 17, I and L improve 14-hydrocodeine production relative to 14HC_P450_36. At position 58, all amino acids tested improve 14-hydroxycodeine production relative to 14HC_P450_36 (FIG. 53B). However, E58K shows the most improvement of the ones tested. At position 59, D is the best amino acid of the ones tested (FIG. 53C). At position 102, L and M improve 14-hydroxycodeine production relative to 14HC_P450_36. (FIG. 53D). At position 181, G, I, L, M, P, Q, S, and V are improved relative to 14HC_P450_36 (FIG. 53E). At position 188, I is the best amino acid of those tested (FIG. 53F). At position 189, V improves 14-hydroxycodeine production relative to 14HC_P450_36 (FIG. 53G). At position 208, N improves 14-hydroxycodeine production relative to 14HC_P450_36 (FIG. 53H). At position 325, I, M, and V improve 14-hydroxycodeine production relative to 14HC_P450_36 (FIG. 53I).

FIG. 54 demonstrates in vitro production of 14-hydroxycodeinone when codeinone is used as substrate. There is a high level of spontaneous conversion of codeinone to 14-hydroxycodeinone when microsomes from strains expressing 14HC_P450_5 or empty vector used. However, there is significant in vitro production of 14-hydroxycodeinone by 14HC_P450_5 compared to the negative control, particularly at 24 hours.

FIG. 55 demonstrates in vitro production of 14-hydroxycodeine when codeine is used as substrate. There is significant production of 14-hydroxycodeine by 14HC_P450_5 over time compared to the Empty Vector negative control. Note that for this assay, samples were not taken at 3 hours.

FIG. 56 demonstrates in vitro production of oxycodone when hydrocodone is used as substrate. There is significant production of oxycodone by 14HC_P450_5 over time compared to the Empty Vector negative control. There is a background of about 4 nM oxycodone which does not change over the course of the assay.

FIG. 57 depicts in vitro production of oxymorphone when hydromorphone is used as substrate.

### DETAILED DESCRIPTION

The present disclosure provides methods for the production of diverse benzylisoquinoline alkaloids (BIAs) in engineered host cells. The present disclosure further provides compositions of diverse alkaloids produced in engineered host cells. Additionally, the present disclosure provides methods for the expression of one or more proteins providing C-14-hydroxylase activity in host cells engineered with a plurality of heterologous enzymes to produce a diverse benzylisoquinoline alkaloid product from simple starting materials such as sugar and/or L-tyrosine. In certain embodiments, the one more proteins providing C-14-hydroxylase activity is heterologous to the engineered host cell. In some embodiments, the one more proteins providing C-14-hydroxylase activity comprises a cytochrome P450 (P450) protein. Additionally, the present disclosure provides methods for the production of one or more engineered P450 proteins and/or one or more engineered cytochrome P450 reductase (CPR) proteins in engineered host cells. In particular cases, the disclosure provides methods for increasing production of diverse alkaloid products by engineered P450 proteins and/or engineered CPR proteins with particular amino acid mutations that increase activty. In particular cases, the disclosure provides methods for producing benzylisoquinolines, promorphinans, morphinans, protoberberines, protopines, benzophenanthridines, secoberberines, phthalideisoquinolines, aporphines, bisbenzylisoquinolines, nal-opioids, nor-opioids, and others through the increased conversion of precursor BIAs to a benzylisoquinoline alkaloid product in an engineered host cell. In further particular cases, the method comprises engineering the host cell with a plurality of heterologous enzymes to increase the production of BIA precursors, including L-tyrosine and 4-HPAA. In further particular examples, an engineered host cell further comprises inactivating mutations in selected enzymes that result in increased production of diverse benzylisoquinoline alkaloid products or decreased production of byproducts. In further particular examples, an engineered host cell further comprises heterologous expression or overexpression of selected enzymes that result in increased production of diverse benzylisoquinoline alkaloid products or decreased production of byproducts. In further particular cases, the byproducts comprise formaldehyde, tyrosol, phenylethanol, or methionol.

### Benzylisoquinoline Alkaloids (BIAs) of Interest

Host cells which produce BIAs of interest are provided. In some examples, engineered strains of host cells such as the engineered strains of the disclosure provide a platform for producing benzylisoquinoline alkaloids of interest and modifications thereof across several structural classes including, but not limited to, precursor BIAs, benzylisoquinolines, promorphinans, morphinans, protoberberines, protopines, benzophenanthridines, secoberberines, phthalideisoquinolines, aporphines, bisbenzylisoquinolines, nal-opioids, nor-opioids, and others. Each of these classes is meant to include biosynthetic precursors, intermediates, and metabolites thereof, of any convenient member of an engineered host cell biosynthetic pathway that may lead to a member of the class. Non-limiting examples of compounds are given below for each of these structural classes. In some cases, the structure of a given example may or may not be characterized itself as a benzylisoquinoline alkaloid. The present chemical entities are meant to include all possible isomers, including single enantiomers, racemic mixtures, optically pure forms, mixtures of diastereomers, and intermediate mixtures.

Benzylisoquinoline alkaloid precursors may include, but are not limited to, norcoclaurine (NC) and norlaudanosoline (NL), as well as NC and NL precursors, such as tyrosine, tyramine, 4-hydroxyphenylacetaldehyde (4-HPAA), 4-hydroxyphenylpyruvic acid (4-HPPA), L-3,4-dihydroxyphenylalanine (L-DOPA), 3,4-dihydroxyphenylacetaldehyde (3,4-DHPAA), and dopamine. In some embodiments, the one or more BIA precursors are 3,4-dihydroxyphenylacetaldehyde (3,4-DHPAA) and dopamine. In certain instances, the one or more BIA precursors are 4-hydroxyphenylacetaldehyde (4-HPAA) and dopamine. In particular, NL and NC may be synthesized, respectively, from precursor molecules via a Pictet-Spengler condensation reaction, where the reaction may occur spontaneously or may by catalyzed by any convenient enzymes.

Benzylisoquinolines may include, but are not limited to, norcoclaurine, norlaudanosoline, coclaurine, 3′-hydroxycoclaurine, 4′-O-methylnorlaudanosoline, 4′-O-methyl-laudanosoline, N-methylnorcoclaurine, laudanosoline, N-methylcoclaurine, 3′-hydroxy-N-methylcoclaurine, reticuline, norreticuline, papaverine, laudanine, laudanosine, tetrahydropapaverine, 1,2-dihydropapaverine, and orientaline.

Promorphinans may include, but are not limited to, salutaridine, salutaridinol, and salutaridinol-7-O-acetate.

Morphinans may include, but are not limited to, thebaine, codeinone codeine, morphine, morphinone, oripavine, neopinone, neopine, neomorphine, hydrocodone, dihydrocodeine, 14-hydroxycodeinone, oxycodone, 14-hydroxycodeine, hydromorphone, dihydromorphine, dihydroetorphine, ethylmorphine, etorphine, metopon, buprenorphine, pholcodine, heterocodeine, oxymorphone, norcodeinone, northebaine, oripavine, normorphinone, hydromorphine, norhydromorphine, and norhydrocodone.

Protoberberines may include, but are not limited to, scoulerine, cheilanthifoline, stylopine, nandinine, jatrorrhizine, stepholidine, discretamine, cis-N-methylstylopine, tetrahydrocolumbamine, palmatine, tetrahydropalmatine, columbamine, canadine, N-methylcanadine, 1-hydroxycanadine, berberine, N-methyl-ophiocarpine, 1,13-dihydroxy-N-methylcanadine, and 1-hydroxy-10-O-acetyl-N-methylcanadine.

Protopines may include, but are not limited to, protopine, 6-hydroxyprotopine, allocryptopine, cryptopine, muramine, and thalictricine.

Benzophenanthridines may include, but are not limited to, dihydrosanguinarine, sanguinarine, dihydrocheilirubine, cheilirubine, dihydromarcapine, marcapine, and chelerythrine.

Secoberberines may include, but are not limited to, 4′-O-desmethylmacrantaldehyde, 4′-O-desmethylpapaveroxine, 4′-O-desmethyl-3-O-acetylpapaveroxine, papaveroxine, and 3-O-aceteylpapaveroxine.

Phthalideisoquinolines may include, but are not limited to, narcotolinehemiacetal, narcotinehemiacetal, narcotoline, noscapine, adlumidine, adlumine, (+) or (−)-bicuculline, capnoidine, carlumine, corledine, corlumidine, decumbenine, 5′-O-demethylnarcotine, (+) or (−)-α or β-hydrastine, and hypecoumine.

Aporphines may include, but are not limited to, magnoflorine, corytuberine, apomorphine, boldine, isoboldine, isothebaine, isocorytuberine, and glaufine.

Bisbenzylisoquinolines may include, but are not limited to, berbamunine, guattegaumerine, dauricine, and liensinine.

Nal-opioids may include, but are not limited to, naltrexone, naloxone, nalmefene, nalorphine, nalorphine, nalodeine, naldemedine, naloxegol, 6β-naltrexol, naltrindole, methylnaltrexone, methylsamidorphan, alvimopan, axelopran, bevenpran, dinicotinate, levallorphan, samidorphan, buprenorphine, dezocine, eptazocine, butorphanol, levorphanol, nalbuphine, pentazocine, phenazocine, norbinaltorphimine, and diprenorphine.

Nor-opioids may include, but are not limited to, norcodeine, noroxycodone, northebaine, norhydrocodone, nordihydro-codeine, nor-14-hydroxy-codeine, norcodeinone, nor-14-hydroxy-codeinone, normorphine, noroxymorphone, nororipavine, norhydro-morphone, nordihydro-morphine, nor-14-hydroxy-morphine, normorphinone, and nor-14-hydroxy-morphinone.

Other compounds that may be produced by the engineered strains of the disclosure may include, but are not limited to, rhoeadine, pavine, isopavine, and cularine.

In certain embodiments, the engineered strains of the disclosure may provide a platform for producing compounds related to tetrahydrobiopterin synthesis including, but not limited to, dihydroneopterin triphosphate, 6-pyruvoyl tetrahydropterin, 5,6,7,8-tetrahydrobiopterin, 7,8-dihydrobiopterin, tetrahydrobiopterin 4a-carbinolamine, quinonoid dihydrobiopterin, and biopterin.

### Host Cells

Any convenient cells may be utilized in the subject host cells and methods. In some cases, the host cells are non-plant cells. In some instances, the host cells may be characterized as microbial cells. In certain cases, the host cells are insect cells, mammalian cells, bacterial cells, fungal cells, or yeast cells. Any convenient type of host cell may be utilized in producing the subject BIA-producing cells, see, e.g., US2008/0176754, US2014/0273109, PCT/US2014/063738, PCT/US2016/030808, PCT/US2015/060891, PCT/US2016/031506, and PCT/US2017/057237, the disclosures of which are incorporated by reference in their entirety. Host cells of interest include, but are not limited to, bacterial cells, such as Bacillus subtilis, Escherichia coli, Streptomyces, Anabaena, Arthrobacter, Acetobacter, Acetobacterium, Bacillus, Bifidobacterium, Brachybacterium, Brevibacterium, Carnobacterium, Clostridium, Corynebacterium, Enterobacter, Escherichia, Gluconacetobacter, Gluconobacter, Hafnia, Halomonas, Klebsiella, Kocuria, Lactobacillus, Leucononstoc, Macrococcus, Methylomonas, Methylobacter, Methylocella, Methylococcus, Microbacterium, Micrococcus, Microcystis, Moorella, Oenococcus, Pediococcus, Prochlorococcus, Propionibacterium, Proteus, Pseudoalteromonas, Pseudomonas, Psychrobacter, Rhodobacter, Rhodococcus, Rhodopseudomonas, Serratia, Staphylococcus, Streptococcus, Streptomyces, Synechococcus, Synechocystis, Tetragenococcus, Weissella, Zymomonas, and Salmonella typhimuium cells, insect cells such as Drosophila melanogaster S2 and Spodoptera frugiperda Sf9 cells, and yeast cells such as Saccharomyces cerevisiae, Schizosaccharomyces pombe, and Pichia pastoris cells. In some examples, the host cells are yeast cells or E. coli cells. In some cases, the host cell is a yeast cell. In some instances, the host cell is from a strain of yeast engineered to express one or more heterologous coding sequences, genes, and/or enzymes. As used herein, “heterologous” means that the material does not naturally occur in the source in which it is introduced or otherwise present. For example, a heterologous nucleotide sequence is a nucleotide sequence that is not naturally present in the host organism. Such a heterologous nucleotide sequence may be used to express a peptide or protein (e.g., an enzyme that is heterologous, i.e., not naturally present in the host organism. A heterologous nucleotide sequence may also be a nucleotide sequence that contains nucleotide sequences that are naturally present in the host organism, but which have been configured or arranged in a manner that does not naturally occur in the host organism. In some instances, the host cell is from a strain of yeast engineered to produce a BIA of interest, such as a 14-hydroxylated benzylisoquinoline alkaloid. In some instances, the host cell is from a strain of yeast engineered to express enzymes of interest. In some instances, the host cell is from a strain of yeast engineered to express an enzyme providing 14-hydroxylase activity. Additionally, in some embodiments a heterologous enzyme providing 14-hydroxylase activity may be able to more efficiently convert a benzylisoquinoline alkaloid to a 14-hydroxylated benzylisoquinoline alkaloid relative to an endogenous enzyme providing 14-hydroxylase activity and/or a wildtype 14-hydroxylase. In some embodiments, a heterologous enzyme providing 14-hydroxylase activity may be substantially similar to a 14-hydroxylase that naturally occurs in another species or organism, but which is heterologous to the host cell of interest. In some cases, a heterologous enzyme providing 14-hydroxylase activity may have an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more similar to an amino acid sequence of a 14-hydroxylase that naturally occurs in another species or organism, but which is heterologous to the host cell of interest. In some instances, the host cell is from a strain of yeast engineered to express a CPR enzyme. Additionally, in some embodiments the host cell engineered to express a CPR enzyme comprises a heterologous CPR enzyme. In some instances, the host cell engineered to express a CPR enzyme may be able to more efficiently convert a benzylisoquinoline alkaloid to a 14-hydroxylated benzylisoquinoline alkaloid relative to an endogenous CPR enzyme and/or a wildtype CPR enzyme. In some embodiments, a heterologous CPR enzyme may be substantially similar to an endogenous CPR enzyme and/or a wildtype CPR enzyme. In some cases, a heterologous CPR enzyme may have an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more similar to an amino acid sequence of an endogenous CPR enzyme and/or a wildtype CPR enzyme. In some instances, the host cell is from a strain of yeast engineered to express an enzyme providing 14-hydroxylase activity and a CPR enzyme.

In some instances, the host cell is a fungal cell. In certain embodiments, the fungal cells may be of the Aspergillus species and strains include Aspergillus niger (ATCC 1015, ATCC 9029, CBS 513.88), Aspergillus oryzae (ATCC 56747, RIB40), Aspergillus terreus (NIH 2624, ATCC 20542) and Aspergillus nidulans (FGSC A4). In certain embodiments, the fungal cells may be of the Rhizopus species, the Trichoderma species, or the Penicillium species. In certain embodiments, the fungal cells may be a yeast cell.

In some instances, the host cell is from a strain of yeast engineered to express a thebaine synthase. The thebaine synthase may be able to more efficiently convert a salutaridinol-7-O-acetate to a thebaine relative to a spontaneous reaction. In some instances, the host cell is from a strain of yeast engineered to produce an engineered thebaine synthase. In some embodiments, an engineered thebaine synthase may be an engineered fusion enzyme. Additionally, the engineered thebaine synthase may be able to more efficiently convert a salutaridinol-7-O-acetate to a thebaine relative to a parent thebaine synthase. In some embodiments, the parent thebaine synthase may be a wildtype thebaine synthase. In some embodiments, a parent thebaine synthase may be substantially similar to a wildtype thebaine synthase. In some cases, a parent thebaine synthase that is substantially similar to a wild-type thebaine synthase may have an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more similar to an amino acid sequence of a wild-type thebaine synthase. The engineered thebaine synthase may be engineered as a fusion enzyme to another enzyme to more efficiently convert a salutaridinol-7-O-acetate to a thebaine relative to the parent thebaine synthase.

In some instances, the host cell is from a strain of yeast engineered to produce a neopinone isomerase. The neopinone isomerase may be able to more efficiently convert a neopinone to a codeinone relative to a spontaneous reaction. In some instances, the host cell is from a strain of yeast engineered to produce an engineered neopinone isomerase. In some embodiments, an engineered neopinone isomerase may be an engineered fusion enzyme. Additionally, the engineered neopinone isomerase may be able to more efficiently convert a neopinone to a codeinone relative to a parent neopinone isomerase. In some embodiments, the parent neopinone isomerase may be a wildtype neopinone isomerase. In some embodiments, a parent neopinone isomerase may be substantially similar to a wildtype neopinone isomerase. In some cases, a parent neopinone isomerase that is substantially similar to a wild-type neopinone isomerase may have an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more similar to an amino acid sequence of a wild-type neopinone isomerase. The engineered neopinone isomerase may be engineered as a fusion enzyme to another enzyme to more efficiently convert a neopinone to a codeinone relative to the parent neopinone isomerase.

In some instances, the host cell is from a strain of yeast engineered to produce an engineered norcoclaurine synthase. Additionally, the engineered norcoclaurine synthase may be able to more efficiency convert a 4-HPAA and dopamine to a norcoclaurine relative to a parent norcoclaurine synthase. Additionally, the engineered norcoclaurine synthase may be able to more efficiently convert a 3,4-DHPA and dopamine to a norlaudanosoline relative to a parent norcoclaurine synthase. In some embodiments, the parent norcoclaurine synthase may be a wildtype norcoclaurine synthase. In some embodiments, a parent norcoclaurine synthase may be substantially similar to a wildtype norcoclaurine synthase. In some cases, a parent norcoclaurine synthase that is substantially similar to a wild-type norcoclaurine synthase may have an amino acid sequence that is at least 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more similar to an amino acid sequence of a wild-type norcoclaurine synthase.

Any of the host cells described in US2008/0176754, US2014/0273109, PCTUS2014/063738, PCT/US2016/030808, PCT/US2015/060891, PCT/US2016/031506, PCT/US2017/057237, International Patent Publication No. WO2022/109194A1, and U.S. Provisional Application No. 62/628,264 by Smolke et al. may be adapted for use in the subject cells and methods. In certain embodiments, the yeast cells may be of the species Saccharomyces cerevisiae (S. cerevisiae). In certain embodiments, the yeast cells may be of the species Schizosaccharomyces pombe. In certain embodiments, the yeast cells may be of the species Pichia pastoris. Yeast is of interest as a host cell because cytochrome P450 proteins are able to fold properly into the endoplasmic reticulum membrane so that their activity is maintained. In some examples, cytochrome P450 proteins are involved in some biosynthetic pathways of interest. In additional examples, cytochrome P450 proteins are involved in the production of BIAs of interest. In further examples, cytochrome P450 proteins are involved in the production of an enzyme of interest.

Yeast strains of interest that find use in the disclosure include, but are not limited to, CEN.PK (Genotype: MATa/α ura3-52 ura3-52 trp1-289 trp1-289 leu2-3_112 leu2-3_112 his3 Δ1 his3 Δ1 MAL2-8C/MAL2-8C SUC2 SUC2), S288C, W303, D273-10B, X2180, A364A, Σ1278B, AB972, SK1, and FL100. In certain cases, the yeast strain is any of S288C (MATα; SUC2 mal mel gal2 CUP1 flo1 flo8-1 hap1), BY4741 (MATα; his3A1; leu2Δ0; met15Δ0; ura3Δ0), BY4742 (MATα; his3A1; leu2Δ0; lys2Δ0; ura3Δ0), BY4743 (MATα/MATα; his3A1/his3A1; leu2Δ0/leu2Δ0; met15Δ0/MET15; LYS2/lys2Δ0; ura3Δ0/ura3Δ0), and WAT11 or W(R), derivatives of the W303-B strain (MATα; ade2-1; his3-11, -15; leu2-3, -112; ura3-1; canR; cyr+) which express the Arabidopsis thaliana NADPH-P450 reductase ATR1 and the yeast NADPH-P450 reductase CPR1, respectively. In another embodiment, the yeast cell is W303alpha (MATα; his3-11,15 trp1-1 leu2-3 ura3-1 ade2-1). The identity and genotype of additional yeast strains of interest may be found at EUROSCARF (web.uni-frankfurt.de/fbl5/mikro/euroscarf/col_index.html).

In certain embodiments, heterologous coding sequences may be codon optimized for expression in Aspergillus sp. and expressed from an appropriate promoter. In certain embodiments, the promoter may be selected from phosphoglycerate kinase promoter (PGK), MbfA promoter, cytochrome c oxidase subunit promoter (CoxA), SrpB promoter, TvdA promoter, malate dehydrogenase promoter (MdhA), beta-mannosidase promoter (ManB). In certain embodiments, a terminator may be selected from glucoamylase terminator (GlaA) or TrpC terminator. In certain embodiments, the expression cassette consisting of a promoter, heterologous coding sequence, and terminator may be expressed from a plasmid or integrated into the genome of the host. In certain embodiments, selection of cells maintaining the plasmid or integration cassette may be performed with antibiotic selection such as hygromycin or nitrogen source utilization, such as using acetamide as a sole nitrogen source. In certain embodiments, DNA constructs may be introduced into the host cells using established transformation methods such as protoplast transformation, lithium acetate, or electroporation. In certain embodiments, cells may be cultured in liquid ME or solid MEA (3% malt extract, 0.5% peptone, and ±1.5% agar) or in Vogel's minimal medium with or without selection.

In some instances, the host cell is a bacterial cell. The bacterial cell may be selected from any bacterial genus. Examples of genuses from which the bacterial cell may come include Anabaena, Arthrobacter, Acetobacter, Acetobacterium, Bacillus, Bifidobacterium, Brachybacterium, Brevibacterium, Carnobacterium, Clostridium, Corynebacterium, Enterobacter, Escherichia, Gluconacetobacter, Gluconobacter, Hafnia, Halomonas, Klebsiella, Kocuria, Lactobacillus, Leucononstoc, Macrococcus, Methylomonas, Methylobacter, Methylocella, Methylococcus, Microbacterium, Micrococcus, Microcystis, Moorella, Oenococcus, Pediococcus, Prochlorococcus, Propionibacterium, Proteus, Pseudoalteromonas, Pseudomonas, Psychrobacter, Rhodobacter, Rhodococcus, Rhodopseudomonas, Serratia, Staphylococcus, Streptococcus, Streptomyces, Synechococcus, Synechocystis, Tetragenococcus, Weissella, and Zymomonas. Examples of bacterial species which may be used with the methods of this disclosure include Arthrobacter nicotianae, Acetobacter aceti, Arthrobacter arilaitensis, Bacillus cereus, Bacillus coagulans, Bacillus licheniformis, Bacillus pumilus, Bacillus sphaericus, Bacillus stearothermophilus, Bacillus subtilis, Bifidobacterium adolescentis, Brachybacterium tyrofermentans, Brevibacterium linens, Carnobacterium divergens, Corynebacterium flavescens, Enterococcus faecium, Gluconacetobacter europaeus, Gluconacetobacter johannae, Gluconobacter oxydans, Hafnia alvei, Halomonas elongata, Kocuria rhizophila, Lactobacillus acidifarinae, Lactobacillus jensenii, Lactococcus lactis, Lactobacillus yamanashiensis, Leuconostoc citreum, Macrococcus caseolyticus, Microbacterium foliorum, Micrococcus lylae, Oenococcus oeni, Pediococcus acidilactici, Propionibacterium acidipropionici, Proteus vulgaris, Pseudomonas fluorescens, Psychrobacter celer, Staphylococcus condimenti, Streptococcus thermophilus, Streptomyces griseus, Tetragenococcus halophilus, Weissella cibaria, Weissella koreensis, Zymomonas mobilis, Corynebacterium glutamicum, Bifidobacterium bifidum/breve/longum, Streptomyces lividans, Streptomyces coelicolor, Lactobacillus plantarum, Lactobacillus sakei, Lactobacillus casei, Pseudoalteromonas citrea, Pseudomonas putida, Clostridium ljungdahlii/aceticum/acetobutylicum/beijerinckii/butyricum, and Moorella themocellum/thermoacetica.

In certain embodiments, the bacterial cells may be of a strain of Escherichia coli. In certain embodiments, the strain of E. coli may be selected from BL21, DH5a, XL1-Blue, HB101, BL21, and K12. In certain embodiments, heterologous coding sequences may be codon optimized for expression in E. coli and expressed from an appropriate promoter. In certain embodiments, the promoter may be selected from T7 promoter, tac promoter, trc promoter, tetracycline-inducible promoter (tet),lac operon promoter (lac), lacO1 promoter. In certain embodiments, the expression cassette consisting of a promoter, heterologous coding sequence, and terminator may be expressed from a plasmid or integrated into the genome. In certain embodiments, the plasmid is selected from pUC19 or pBAD. In certain embodiments, selection of cells maintaining the plasmid or integration cassette may be performed with antibiotic selection such as kanamycin, chloramphenicol, streptomycin, spectinomycin, gentamycin, erythromycin, or ampicillin. In certain embodiments, DNA constructs may be introduced into the host cells using established transformation methods such as conjugation, heat shock chemical transformation, or electroporation. In certain embodiments, cells may be cultured in liquid Luria-Bertani (LB) media at about 37° C. with or without antibiotics.

In certain embodiments, the bacterial cells may be a strain of Bacillus subtilis. In certain embodiments, the strain of B. subtilis may be selected from 1779, GP25, RO-NN-1, 168, BSn5, BEST195, 1A382, and 62178. In certain embodiments, heterologous coding sequences may be codon optimized for expression in Bacillus sp. and expressed from an appropriate promoter. In certain embodiments, the promoter may be selected from grac promoter, p43 promoter, or trnQ promoter. In certain embodiments, the expression cassette consisting of the promoter, heterologous coding sequence, and terminator may be expressed from a plasmid or integrated into the genome. In certain embodiments, the plasmid is selected from pHP13 pE194, pC194, pHT01, or pHT43. In certain embodiments, integrating vectors such as pDG364 or pDG1730 may be used to integrate the expression cassette into the genome. In certain embodiments, selection of cells maintaining the plasmid or integration cassette may be performed with antibiotic selection such as erythromycin, kanamycin, tetracycline, and spectinomycin. In certain embodiments, DNA constructs may be introduced into the host cells using established transformation methods such as natural competence, heat shock, or chemical transformation. In certain embodiments, cells may be cultured in liquid Luria-Bertani (LB) media at 37° C. or M9 medium plus glucose and tryptophan.

### Genetic Modifications to Host Cells

The host cells may be engineered to include one or more modifications (such as two or more, three or more, four or more, five or more, or even more modifications) that provide for the production of BIAs of interest. Additionally or alternatively, the host cells may be engineered to include one or more modifications (such as two or more, three or more, four or more, five or more, or even more modifications) that provide for the production of enzymes of interest. In some cases, a modification is a genetic modification, such as a mutation, addition, or deletion of a gene or fragment thereof, or transcription regulation of a gene or fragment thereof. As used herein, the term “mutation” refers to a deletion, insertion, or substitution of an amino acid(s) residue or nucleotide(s) residue relative to a reference sequence or motif. The mutation may be incorporated as a directed mutation to the native gene at the original locus. In some cases, the mutation may be incorporated as an additional copy of the gene introduced as a genetic integration at a separate locus, or as an additional copy on an episomal vector such as a 2μ or centromeric plasmid. In certain instances, the substrate inhibited copy of the enzyme is under the native cell transcriptional regulation. In some instances, the substrate inhibited copy of the enzyme is introduced with engineered constitutive or dynamic regulation of protein expression by placing it under the control of a synthetic promoter. In some examples, the object of one or more modifications may be a native gene. In some examples, the object of one or more modifications may be a non-native gene. In some examples, a non-native gene may be inserted into a host cell. In further examples, a non-native gene may be altered by one or more modifications prior to being inserted into a host cell.

An engineered host cell may overproduce one or more BIAs of interest. By overproduce is meant that the cell has an improved or increased production of a BIA molecule of interest relative to a control cell (e.g., an unmodified cell). By improved or increased production is meant both the production of some amount of the BIA of interest where the control has no BIA of interest production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some BIA of interest production. An engineered host cell may further overproduce one or more morphinan alkaloids. In some cases, the engineered host cell may produce some amount of the morphinan alkaloid of interest where the control has no morphinan alkaloid production, as well as an increase of about 1% or more, about 2% or more, about 3% or more, about 4% or more, about 5% or more, about 6% or more, about 7% or more, about 8% or more, about 9% or more, about 10% or more, about 20% or more, about 30% or more, about 40% or more, about 50% or more, about 60% or more, about 80% or more, about 100% or more, about 1%, about 2%, about 3%, about 4%, about 5%, about 6%, about 7%, about 8%, about 9%, about 10%, about 20%, about 30%, about 40%, about 50%, about 60%, about 80%, about 100%, about 1% to about 10%, about 5% to about 15%, about 15% to about 25%, about 25% to about 35%, about 35% to about 45%, about 45% to about 55%, about 55% to about 65%, about 65% to about 75%, about 75% to about 85%, about 85% to about 95%, about 2-fold, about 5-fold, about 10-fold, about 20-fold, about 30-fold, about 40-fold, about 50-fold, about 60-fold, about 70-fold, about 80-fold, about 90-fold, about 100 fold, about 110-fold, about 120-fold, about 130-fold, about 140-fold, about 150-fold, about 160-fold, about 170-fold, about 180-fold, about 190-fold, about 200-fold, or more than 200-fold in situations where the control host cell has some baseline morphinan alkaloid of interest production. In some cases, the morphinan alkaloid is formed from a 1-benzylisoquinoline alkaloid product, or derivative thereof, of a C-14 hydroxylation reaction catalyzed by an engineered C-14 hydroxylase and/or an engineered CPR within an engineered host cell. The engineered C-14 hydroxylase and/or engineered CPR may comprise two separate enzymes that work to produce a C-14 hydroxylase and/or engineered CPR reaction. An engineered host cell may further overproduce one or more of a promorphinan, a nor-opioid, or a morphinan alkaloid.

In some cases, the engineered host cell is capable of producing an increased amount of thebaine relative to a control host cell that lacks the one or more modifications (e.g., as described herein). In some cases, the engineered host cell having a thebaine synthase is capable of producing an increased amount of thebaine relative to a host cell that lacks a thebaine synthase. In some cases, the engineered host cell having an engineered thebaine synthase is capable of producing an increased amount of thebaine relative to a host cell having a wildtype thebaine synthase (e.g., as described herein). In certain instances, the increased amount of thebaine is about 10% or more relative to the control host cell, such as about 20% or more, about 30% or more, about 40% or more, about 50% or more, about 60% or more, about 80% or more, about 100% or more, about 2-fold or more, about 5-fold or more, or even about 10-fold or more relative to the control host cell. In some cases, thebaine is the product of a thebaine synthase reaction within an engineered host cell. In some cases, thebaine is the product of a thebaine synthase reaction catalyzed by at least one engineered thebaine synthase within an engineered host cell. In these cases, salutaridinol-7-O-acetate may be the substrate of the thebaine synthase reaction.

In some cases, the engineered host cell is capable of producing an increased amount of codeinone, or morphinan alkaloid product downstream from codeinone in a biosynthetic pathway, relative to a control host cell that lacks the one or more modifications (e.g., as described herein). In some cases, the engineered host cell having a neopinone isomerase is capable of producing an increased amount of codeinone, or morphinan alkaloid product downstream from codeinone in a biosynthetic pathway, relative to a host cell that lacks a neopinone isomerase. In some cases, the engineered host cell having an engineered neopinone isomerase is capable of producing an increased amount of codeinone, or morphinan alkaloid product downstream from codeinone in a biosynthetic pathway, relative to a host cell having a parent neopinone isomerase (e.g., as described herein). In certain instances, the increased amount of codeinone, or morphinan alkaloid product downstream from codeinone in a biosynthetic pathway, is about about 1% or more, about 2% or more, about 3% or more, about 4% or more, about 5% or more, about 6% or more, about 7% or more, about 8% or more, about 9% or more, about 10% or more, about 20% or more, about 30% or more, about 40% or more, about 50% or more, about 60% or more, about 80% or more, about 100% or more, about 1%, about 2%, about 3%, about 4%, about 5%, about 6%, about 7%, about 8%, about 9%, about 10%, about 20%, about 30%, about 40%, about 50%, about 60%, about 80%, about 100%, about 1% to about 10%, about 5% to about 15%, about 15% to about 25%, about 25% to about 35%, about 35% to about 45%, about 45% to about 55%, about 55% to about 65%, about 65% to about 75%, about 75% to about 85%, about 85% to about 95%, about 2-fold, about 5-fold, about 10-fold, about 20-fold, about 30-fold, about 40-fold, about 50-fold, about 60-fold, about 70-fold, about 80-fold, about 90-fold, about 100 fold, about 110-fold, about 120-fold, about 130-fold, about 140-fold, about 150-fold, about 160-fold, about 170-fold, about 180-fold, about 190-fold, about 200-fold, or more than 200-fold relative to the control host cell. In some cases, codeinone is the product of a neopinone isomerase reaction within an engineered host cell. In some cases, codeinone is the product of a neopinone isomerase reaction catalyzed by at least one engineered neopinone isomerase within an engineered host cell. In these cases, neopinone may be the substrate of the neopinone isomerase reaction.

Additionally, an engineered host cell may overproduce one or more enzymes of interest. By overproduce is meant that the cell has an improved or increased production of an enzyme of interest relative to a control host cell (e.g., an unmodified cell). By improved or increased production is meant both the production of some amount of the enzyme of interest where the control has no production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control host cell has some enzyme of interest production.

An engineered host cell may overproduce one or more thebaine synthase enzymes. In some cases, the engineered host cell may produce some amount of the thebaine synthase enzyme where the control host cell has no thebaine synthase enzyme production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some thebaine synthase enzyme production.

An engineered host cell may overproduce one or more engineered thebaine synthase enzymes. In some cases, the engineered host cell may produce some amount of the engineered thebaine synthase where the control host cell has no thebaine synthase enzyme production, or where the control host cell has a same level of production of wild-type thebaine synthase in comparison to the engineered host cell, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some thebaine synthase enzyme production. In some cases, an engineered thebaine synthase may be an engineered fusion enzyme.

An engineered host cell may overproduce one or more neopinone isomerase enzymes. In some cases, the engineered host cell may produce some amount of the neopinone isomerase enzyme where the control has no neopinone isomerase enzyme production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some neopinone isomerase enzyme production.

An engineered host cell may overproduce one or more engineered neopinone isomerase enzymes. In some cases, the engineered host cell may produce some amount of the engineered neopinone isomerase where the control has no neopinone isomerase enzyme production, or where the control has a same level of production of wild-type neopinone isomerase in comparison to the engineered host cell, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some neopinone isomerase enzyme production. In some cases, an engineered neopinone isomerase may be an engineered fusion enzyme.

An engineered host cell may further overproduce one or more enzymes that are derived from the neopinone isomerase enzyme. In some cases, the engineered host cell may produce some amount of the enzymes that are derived from the neopinone isomerase enzyme, where the control has no production of enzymes that are derived from the neopinone isomerase enzyme, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some production of enzymes that are derived from the neopinone isomerase enzyme

An engineered host cell may overproduce one or more engineered norcoclaurine synthase enzymes. In some cases, the engineered host cell may produce some amount of the engineered norcoclaurine synthase where the control has no norcoclaurine synthase enzyme production, or where the control has a same level of production of wild-type norcoclaurine synthase in comparison to the engineered host cell, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some norcoclaurine synthase enzyme production.

An engineered host cell may overproduce one or more enzymes providing C-14-hydroxylase activity. In some cases, the engineered host cell may produce some amount of the enzyme providing C-14-hydroxylase activity where the control host cell has no production of an enzyme providing C-14-hydroxylase activity, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control host cell has some production of an enzyme providing C-14-hydroxylase activity. In some embodiments, the enzyme providing C-14-hydroxylase activity is a P450.

An engineered host cell may overproduce one or more engineered enzymes providing C-14-hydroxylase activity. In some cases, the engineered host cell may produce some amount of the engineered enzyme providing C-14-hydroxylase activity where the control host cell produces no enzyme providing C-14-hydroxylase activity, or where the control host cell has a same level of production of a wild-type enzyme providing C-14-hydroxylase activity in comparison to the engineered host cell, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control host cell has some enzyme providing C-14-hydroxylase activity production. In some cases, an engineered enzyme providing C-14-hydroxylase activity may be an engineered fusion enzyme. In some embodiments, the enzyme providing C-14-hydroxylase activity is a P450.

An engineered host cell may further overproduce one or more enzymes that are derived from an enzyme providing C-14-hydroxylase activity. In some cases, the engineered host cell may produce some amount of the enzymes that are derived from an enzyme providing C-14-hydroxylase activity, where the control host cell has no production of enzymes that are derived from the enzyme providing C-14-hydroxylase activity, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control host cell has some production of enzymes that are derived from the enzyme providing C-14-hydroxylase activity. In some embodiments, the enzyme providing C-14-hydroxylase activity is a P450.

An engineered host cell may overproduce one or more CPR enzymes. In some cases, the engineered host cell may produce some amount of the CPR enzyme where the control host cell has no CPR enzyme production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control host cell has some production of the CPR enzyme.

An engineered host cell may overproduce one or more engineered CPR enzymes. In some cases, the engineered host cell may produce some amount of the engineered CPR enzyme where the control host cell produces no CPR enzyme production, or where the control host cell has a same level of production of a wild-type CPR enzyme in comparison to the engineered host cell, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control host cell has some CPR enzyme production. In some cases, an engineered CPR enzyme may be an engineered fusion enzyme.

An engineered host cell may further overproduce one or more enzymes that are derived from the CPR enzyme. In some cases, the engineered host cell may produce some amount of the enzymes that are derived from the CPR enzyme, where the control host cell has no production of enzymes that are derived from the CPR enzyme, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control host cell has some production of enzymes that are derived from the CPR enzyme.

Additionally, an engineered host cell may overproduce one or more 14-hydroxylated morphanin BIA products and/or intermediates. In particular, an engineered host cell is capable of producing an increased amount of C-14-hydroxylated morphanin BIA products and/or intermediates to a control host cell that lacks the one or more modifications (e.g., as described herein), including modifications related to harboring an engineered C-14 hydroxylase and an engineered CPR. In certain instances, the increased amount of C-14-hydroxylated morphanin BIA products and/or intermediates is about 1% or more, about 2% or more, about 3% or more, about 4% or more, about 5% or more, about 6% or more, about 7% or more, about 8% or more, about 9% or more, about 10% or more, about 20% or more, about 30% or more, about 40% or more, about 50% or more, about 60% or more, about 80% or more, about 100% or more, about 1%, about 2%, about 3%, about 4%, about 5%, about 6%, about 7%, about 8%, about 9%, about 10%, about 20%, about 30%, about 40%, about 50%, about 60%, about 80%, about 100%, about 1% to about 10%, about 5% to about 15%, about 15% to about 25%, about 25% to about 35%, about 35% to about 45%, about 45% to about 55%, about 55% to about 65%, about 65% to about 75%, about 75% to about 85%, about 85% to about 95%, about 2-fold, about 5-fold, about 10-fold, about 20-fold, about 30-fold, about 40-fold, about 50-fold, about 60-fold, about 70-fold, about 80-fold, about 90-fold, about 100 fold, about 110-fold, about 120-fold, about 130-fold, about 140-fold, about 150-fold, about 160-fold, about 170-fold, about 180-fold, about 190-fold, about 200-fold, or more than 200-fold relative to the control host cell. In some cases, the one or more C-14-hydroxylated morphanin BIA products and/or intermediates are formed from at least one BIA monomer that is the product, or derivative thereof, of a C-14 hydroxylation reaction catalyzed by an engineered epimerase within an engineered host cell. The engineered C-14 hydroxylase and engineered CPR may comprise two separate enzymes that work to produce a C-14 hydroxylation reaction. An engineered host cell may further overproduce one or more of codeinone, codeine, morphine, morphinone, oripavine, neopinone, neopine, neomorphine, hydrocodone, dihydrocodeine, 14-hydroxycodeinone, 14-hydroxyheterocodeine, oxycodone, 14-hydroxycodeine, hydromorphone, dihydromorphine, dihydroetorphine, ethylmorphine, etorphine, metopon, buprenorphine, pholcodine, heterocodeine, oxymorphone, noroxymorphone, norcodeinone, northebaine, nororipavine, normorphinone, hydromorphine, norhydromorphine, norheterocodeine, norhydromorphone, noroxymorphone, and norhydrocodone. In certain embodiments, an engineered host cell may further overproduce a nor or a 14-hydroxy derivative of a morphinan. In certain embodiments, an engineered host cell may further overproduce noroxymorphone. In certain embodiments, an engineered host cell may further overproduce 14-hydroxycodeinone. In certain embodiments, an engineered host cell may further overproduce 14-hydroxycodeine.

In some cases, the one or more (such as two or more, three or more, or four or more) modifications may be selected from: an engineered thebaine synthase modification; an engineered neopinone isomerase modification; an engineered norcoclaurine synthase modification; an enzyme expression modification; an inactivation modification; a C-14-hydroxylase modification; a CPR modification; and a byproduct inhibition alleviating modification, or a combination thereof. A cell that includes one or more modifications may be referred to as an engineered cell.

### Substrate Inhibition Alleviating Mutations

In some instances, the engineered host cells are cells that include one or more substrate inhibition alleviating mutations (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “substrate inhibition alleviating mutation” refers to a mutation that alleviates a substrate inhibition control mechanism of the cell.

A mutation that alleviates substrate inhibition reduces the inhibition of a regulated enzyme in the cell of interest relative to a control cell and provides for an increased level of the regulated compound or a downstream biosynthetic product thereof. In some cases, by alleviating inhibition of the regulated enzyme is meant that the IC₅₀ of inhibition is increased by 2-fold or more, such as by 3-fold or more, 5-fold or more, 10-fold or more, 30-fold or more, 100-fold or more, 300-fold or more, 1000-fold or more, or even more. By increased level is meant a level that is 110% or more of that of the regulated compound in a control cell or a downstream product thereof, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the regulated compound in the engineered host cell or a downstream product thereof.

A variety of substrate inhibition control mechanisms and biosynthetic enzymes in the engineered host cell that are directed to regulation of levels of BIAs of interest, or precursors thereof, may be targeted for substrate inhibition alleviation. The engineered host cell may include one or more substrate inhibition alleviating mutations in one or more biosynthetic enzyme genes. The one or more mutations may be located in any convenient biosynthetic enzyme genes where the biosynthetic enzyme is subject to regulatory control. In some embodiments, the one or more biosynthetic enzyme genes encode one or more tyrosine hydroxylase enzymes. In certain instances, the one or more substrate inhibition alleviating mutations are present in a biosynthetic enzyme gene that is TyrH. In some embodiments, the engineered host cell may include one or more substrate inhibition alleviating mutations in one or more biosynthetic enzyme genes such as one of those genes described in Table 11.

In certain embodiments, the one or more substrate inhibition alleviating mutations are present in the TyrH gene. The TyrH gene encodes tyrosine hydroxylase, which is an enzyme that converts tyrosine to L-DOPA. However, TyrH is inhibited by its substrate, tyrosine. Mammalian tyrosine hydroxylase activity, such as that seen in humans or rats, can be improved through mutations to the TyrH gene that relieve substrate inhibition. In particular, substrate inhibition from tyrosine can be relieved by a point mutation W166Y in the TyrH gene. The point mutation W166Y in the TyrH gene may also improve the binding of the cosubstrate of tyrosine hydroxylase, BH₄, to catalyze the reaction of tyrosine to L-DOPA. The mutants of TyrH, when expressed in yeast strains to produce BIAs from sugar (such as those described in U.S. Provisional Patent Application Ser. No. 61/899,496) can significantly improve the production of BIAs.

Any convenient numbers and types of mutations may be utilized to alleviate a substrate inhibition control mechanism. In certain embodiments, the engineered host cells of the present disclosure may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more substrate inhibition alleviating mutations, such as 1, 2, 3, 4, 5, 6, 7, 8, 9, 10, 11, 12, 13, 14 or 15 substrate inhibition alleviating mutations in one or more biosynthetic enzyme genes within the engineered host cell.

### Cofactor Recovery Promoting Mechanisms

In some instances, the engineered host cells are cells that include one or more cofactor recovery promoting mechanisms (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “cofactor recovery promoting mechanism” refers to a mechanism that promotes a cofactor recovery control mechanism of the cell.

A variety of cofactor recovery control mechanisms and biosynthetic enzymes in the engineered host cell that are directed to regulation of levels of BIAs of interest, or precursors thereof, may be targeted for cofactor recovery promotion. The engineered host cell may include one or more cofactor recovery promoting mechanism in one or more biosynthetic enzyme genes. In some examples, the engineered host cell may include a heterologous coding sequence that encodes dihydrofolate reductase (DHFR). When DHFR is expressed, it may convert 7,8-dihydrobiopterin (BH₂) to the tetrahydrobiopterin (BH₄), thereby recovering BH₄ as a TyrH cosubstrate. In some examples, the engineered host cell may include one or more cofactor recovery promoting mechanisms in one or more biosynthetic enzyme genes such as one of those genes described in Table 11.

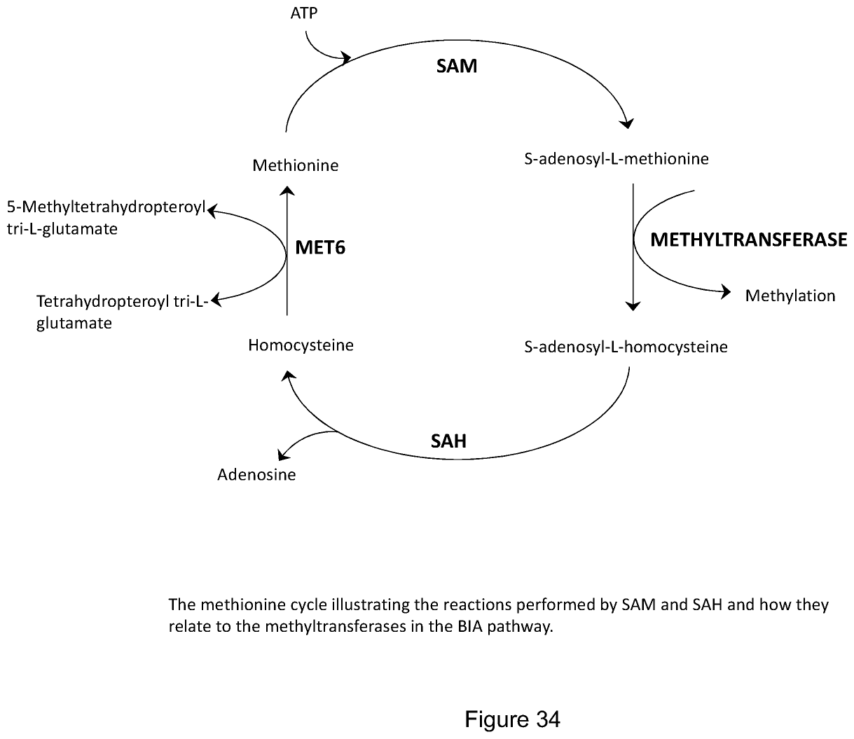

One important cofactor for production of BIAs of interest is S-adenosyl-L-methionine (SAM) used by multiple methyltransferase enzymes. As SAM is utilized in this reaction, it is converted to S-adenosyl-L-homocysteine (SAH), homocysteine, methionine, and then back to SAM. This pathway is illustrated in FIG. 34 and may be targeted for modification to increase cofactor recovery. In some examples, the engineered host cell may include overexpression of the native S-adenosyl-L-homocysteine hydrolase (SAH1). In some examples, the engineered host cell may include overexpression of the native methionine synthase (MET6). In some examples, the engineered host cell may include overexpression of the native S-adenosylmethionine synthetase (SAM2). When one or more of these genes is overexpressed, it may increase recovery of SAH to SAM. In some examples, the engineered host cell may include one or more cofactor recycling genes described in Table 11.

Any convenient numbers and types of mechanisms may be utilized to promote a cofactor recovery control mechanism. In certain embodiments, the engineered host cells of the present disclosure may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more cofactor recovery promoting mechanisms such as 1, 2, 3, 4, 5, 6, 7, 8, 9, 10, 11, 12, 13, 14 or 15 cofactor recovery promoting mechanisms in one or more biosynthetic enzyme genes within the engineered host cell.

### Cofactor Recovery Promoting Mechanisms

In some instances, the engineered host cells are cells that include one or more cofactor recovery promoting mechanisms (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “cofactor recovery promoting mechanism” refers to a mechanism that promotes a cofactor recovery control mechanism of the cell.

A variety of cofactor recovery control mechanisms and biosynthetic enzymes in the engineered host cell that are directed to regulation of levels of BIAs of interest, or precursors thereof, may be targeted for cofactor recovery promotion. The engineered host cell may include one or more cofactor recovery promoting mechanism in one or more biosynthetic enzyme genes. In some examples, the engineered host cell may include a heterologous coding sequence that encodes dihydrofolate reductase (DHFR). When DHFR is expressed, it may convert 7,8-dihydrobiopterin (BH₂) to the tetrahydrobiopterin (BH₄), thereby recovering BH₄ as a TyrH cosubstrate. In some examples, the engineered host cell may include one or more cofactor recovery promoting mechanisms in one or more biosynthetic enzyme genes such as one of those genes described in Table 11.

One important cofactor for production of BIAs of interest is S-adenosyl-L-methionine (SAM) used by multiple methyltransferase enzymes. As SAM is utilized in this reaction, it is converted to S-adenosyl-L-homocysteine (SAH), homocysteine, methionine, and then back to SAM. This pathway is illustrated in FIG. 34 and may be targeted for modification to increase cofactor recovery. In some examples, the engineered host cell may include overexpression of the native S-adenosyl-L-homocysteine hydrolase (SAH1). In some examples, the engineered host cell may include overexpression of the native methionine synthase (MET6). In some examples, the engineered host cell may include overexpression of the native S-adenosylmethionine synthetase (SAM2). When one or more of these genes is overexpressed, it may increase recovery of SAH to SAM. In some examples, the engineered host cell may include one or more cofactor recycling genes described in Table 11.

Any convenient numbers and types of mechanisms may be utilized to promote a cofactor recovery control mechanism. In certain embodiments, the engineered host cells of the present disclosure may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more cofactor recovery promoting mechanisms such as 1, 2, 3, 4, 5, 6, 7, 8, 9, 10, 11, 12, 13, 14 or 15 cofactor recovery promoting mechanisms in one or more biosynthetic enzyme genes within the engineered host cell.

### Product Inhibition Alleviating Mutations

In some instances, the engineered host cells are cells that include one or more product inhibition alleviating mutations (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “product inhibition alleviating mutation” refers to a mutation that alleviates a short term and/or long term product inhibition control mechanism of an engineered host cell. Short term product inhibition is a control mechanism of the cell in which there is competitive binding at a cosubstrate binding site. Long term product inhibition is a control mechanism of the cell in which there is irreversible binding of a compound away from a desired pathway.

A mutation that alleviates product inhibition reduces the inhibition of a regulated enzyme in the cell of interest relative to a control cell and provides for an increased level of the regulated compound or a downstream biosynthetic product thereof. In some cases, by alleviating inhibition of the regulated enzyme is meant that the IC₅₀ of inhibition is increased by 2-fold or more, such as by 3-fold or more, 5-fold or more, 10-fold or more, 30-fold or more, 100-fold or more, 300-fold or more, 1000-fold or more, or even more. By increased level is meant a level that is 110% or more of that of the regulated compound in a control cell or a downstream product thereof, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the regulated compound in the engineered host cell or a downstream product thereof.

A variety of product inhibition control mechanisms and biosynthetic enzymes in the engineered host cell that are directed to regulation of levels of BIAs of interest may be targeted for product inhibition alleviation. The engineered host cell may include one or more product inhibition alleviating mutations in one or more biosynthetic enzyme genes. The mutation may be located in any convenient biosynthetic enzyme genes where the biosynthetic enzyme is subject to regulatory control. In some embodiments, the one or more biosynthetic enzyme genes encode one or more tyrosine hydroxylase enzymes. In certain instances, the one or more product inhibition alleviating mutations are present in a biosynthetic enzyme gene that is TyrH. In some embodiments, the engineered host cell includes one or more product inhibition alleviating mutations in one or more biosynthetic enzyme genes such as one of those genes described in Tables 11 and 17.

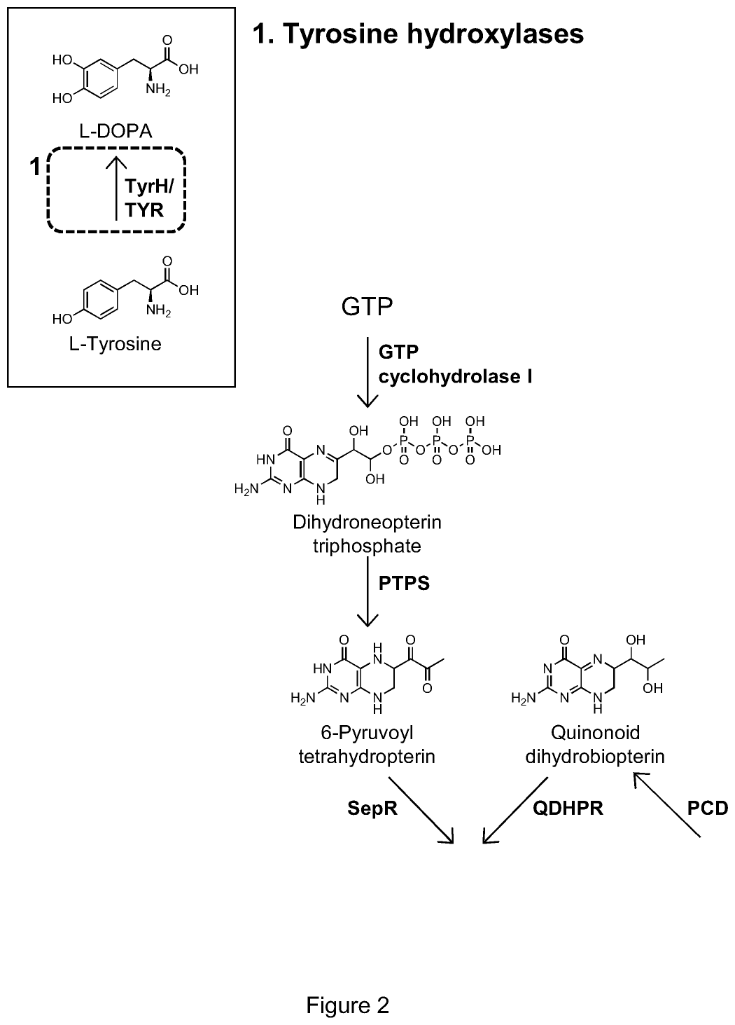

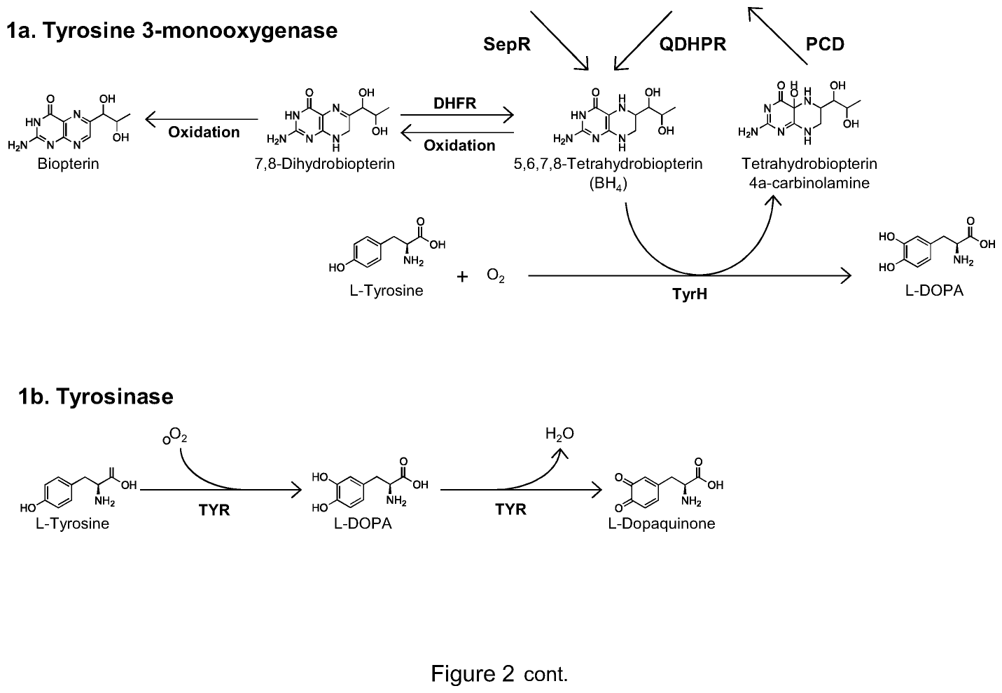

In certain embodiments, the one or more product inhibition alleviating mutations are present in the TyrH gene. The TyrH gene encodes tyrosine hydroxylase, which is an enzyme that converts tyrosine to L-DOPA. TyrH requires tetrahydrobiopterin (BH₄) as a cosubstrate to catalyze the hydroxylation reaction. Some microbial strains, such as Saccharomyces cerevisiae, do not naturally produce BH₄, but can be engineered to produce this substrate through a four-enzyme synthesis and recycling pathway, as illustrated in FIG. 2. FIG. 2 illustrates examples of synthesis, recycling, and salvage pathways of tetrahydrobiopterin, in accordance with some embodiments of the disclosure. FIG. 2 provides the use of the enzymes PTPS, pyruvoyl tetrahydropterin synthase; SepR, sepiapterin reductase; PCD, pterin 4a-carbinolamine dehydratase; QDHPR, dihydropteridine reductase; and DHFR, dihydrofolate reductase. Of the enzymes that are illustrated in FIG. 2, yeast synthesizes an endogenous GTP cyclohydrolase I. GTP and dihydroneopterin triphosphate are naturally synthesized in yeast. Additionally, other metabolites in FIG. 2 are not naturally produced in yeast.

TyrH is inhibited by its product L-DOPA, as well as other catecholamines, particularly dopamine. Mammalian tyrosine hydroxylase activity, such as from humans or rats, can be improved through mutations that relieve product inhibition. For example, short term product inhibition, such as competitive binding at the cosubstrate binding site, can be relieved by a point mutation W166Y on the TyrH gene. In particular, the point mutation W166Y on the TyrH gene may improve binding of the cosubstrate. Additionally, short term product inhibition to relieve competitive binding at the cosubstrate binding site may be improved by a point mutation S40D on the TyrH gene. Short term product inhibition may also be improved by the joint mutations of R37E, R38E on the TyrH gene. In particular, R37E, R38E mutations may together specifically improve tyrosine hydroxylase activity in the presence of dopamine.

Additionally, long term product inhibition may be relieved by point mutations on the TyrH gene. Long term product inhibition relief may include the irreversible binding of catecholamine to iron in the active site such that there is less catecholamine present to act as a product inhibitor of tyrosine hydroxylase activity. Long term product inhibition can be relieved by the mutations E332D and Y371F, respectively, in the TyrH gene.

Combinations of the mutations can be made (such as two or three or more mutations at once) to relieve multiple types of substrate and product inhibition to further improve the activity of TyrH. The mutants of TyrH, when expressed in yeast strains to produce BIAs from sugar (such as those described in U.S. Provisional Patent Application Ser. No. 61/899,496) can significantly improve the production of BIAs.

Any convenient numbers and types of mutations may be utilized to alleviate a product inhibition control mechanism. In certain embodiments, the engineered host cells of the present disclosure may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more product inhibition alleviating mutations, such as 1, 2, 3, 4, 5, 6, 7, 8, 9, 10, 11, 12, 13, 14 or 15 product inhibition alleviating mutations in one or more biosynthetic enzyme genes within the engineered host cell.

### Feedback Inhibition Alleviating Mutations

In some instances, the engineered host cells are cells that include one or more feedback inhibition alleviating mutations (such as two or more, three or more, four or more, five or more, or even more) in one or more biosynthetic enzyme genes of the cell. In some cases, the one or more biosynthetic enzyme genes are native to the cell (e.g., is present in an unmodified cell). Additionally or alternatively, in some examples the one or more biosynthetic enzyme genes are non-native to the cell. As used herein, the term “feedback inhibition alleviating mutation” refers to a mutation that alleviates a feedback inhibition control mechanism of an engineered host cell. Feedback inhibition is a control mechanism of the cell in which an enzyme in the synthetic pathway of a regulated compound is inhibited when that compound has accumulated to a certain level, thereby balancing the amount of the compound in the cell. A mutation that alleviates feedback inhibition reduces the inhibition of a regulated enzyme in the engineered host cell relative to a control cell. In this way, engineered host cell provides for an increased level of the regulated compound or a downstream biosynthetic product thereof. In some cases, by alleviating inhibition of the regulated enzyme is meant that the IC₅₀ of inhibition is increased by 2-fold or more, such as by 3-fold or more, 5-fold or more, 10-fold or more, 30-fold or more, 100-fold or more, 300-fold or more, 1000-fold or more, or even more. By increased level is meant a level that is 110% or more of that of the regulated compound in a control cell or a downstream product thereof, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the regulated compound in the host cell or a downstream product thereof.

A variety of feedback inhibition control mechanisms and biosynthetic enzymes that are directed to regulation of levels of BIAs of interest may be targeted for alleviation in the host cell. The host cell may include one or more feedback inhibition alleviating mutations in one or more biosynthetic enzyme genes native to the cell. The one or more mutations may be located in any convenient biosynthetic enzyme genes where the biosynthetic enzyme is subject to regulatory control. In some embodiments, the one or more biosynthetic enzyme genes may encode one or more enzymes selected from a 3-deoxy-d-arabinose-heptulosonate-7-phosphate (DAHP) synthase and a chorismate mutase. In some embodiments, the one or more biosynthetic enzyme genes encode a 3-deoxy-d-arabinose-heptulosonate-7-phosphate (DAHP) synthase. In some instances, the one or more biosynthetic enzyme genes may encode a chorismate mutase. In certain instances, the one or more feedback inhibition alleviating mutations may be present in a biosynthetic enzyme gene selected from ARO4 and ARO7. In certain instances, the one or more feedback inhibition alleviating mutations may be present in a biosynthetic enzyme gene that is ARO4. In certain instances, the one or more feedback inhibition alleviating mutations are present in a biosynthetic enzyme gene that is ARO7. In some embodiments, the engineered host cell may include one or more feedback inhibition alleviating mutations in one or more biosynthetic enzyme genes such as one of those genes described in Table 11.

Any convenient numbers and types of mutations may be utilized to alleviate a feedback inhibition control mechanism. As used herein, the term “mutation” refers to a deletion, insertion, or substitution of an amino acid(s) residue or nucleotide(s) residue relative to a reference sequence or motif. The mutation may be incorporated as a directed mutation to the native gene at the original locus. In some cases, the mutation may be incorporated as an additional copy of the gene introduced as a genetic integration at a separate locus, or as an additional copy on an episomal vector such as a 2μ or centromeric plasmid. In certain instances, the feedback inhibited copy of the enzyme is under the native cell transcriptional regulation. In some instances, the feedback inhibited copy of the enzyme is introduced with engineered constitutive or dynamic regulation of protein expression by placing it under the control of a synthetic promoter.

In certain embodiments, the one or more feedback inhibition alleviating mutations may be present in the ARO4 gene. ARO4 mutations of interest may include, but are not limited to, substitution of the lysine residue at position 229 with a leucine, a substitution of the glutamine residue at position 166 with a lysine residue, or a mutation as described by Hartmann M, et al. ((2003) Proc Natl Acad Sci USA 100(3):862-867) or Fukuda et al. ((1992) J Ferment Bioeng 74(2):117-119). In some instances, mutations for conferring feedback inhibition may be selected from a mutagenized library of enzyme mutants. Examples of such selections may include rescue of growth of o-fluoro-D,L-phenylalanine or growth of aro3 mutant yeast strains in media with excess tyrosine as described by Fukuda et al. ((1990) Breeding of Brewing Yeast Producing a Large Amount of Beta-Phenylethyl Alcohol and Beta-Phenylethyl Acetate. Agr Biol Chem Tokyo 54(1):269-271).

In certain embodiments, the engineered host cells of the present disclosure may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more feedback inhibition alleviating mutations, such as 1, 2, 3, 4, 5, 6, 7, 8, 9, 10, 11, 12, 13, 14 or 15 feedback inhibition alleviating mutations in one or more biosynthetic enzyme genes within the engineered host cell.

### Byproduct Inhibition Alleviating Modifications

The host cells may include one or more modifications (such as two or more, three or more, four or more, five or more, or even more modifications) of one or more biosynthetic enzyme genes of the cell that are directed to alleviating byproduct inhibition. In some examples, the one or more biosynthetic enzyme genes are native to the cell. In some examples, the one or more biosynthetic enzyme genes are non-native to the cell. Any convenient biosynthetic enzyme genes of the cell may be targeted for modification to alleviate accumulation of key byproducts. As used herein, the term “byproduct inhibition alleviating modification” refers to a modification that reduces the accumulation of a key inhibitory byproduct of an engineered host cell. Byproduct inhibition is a mechanism of the cell in which accumulation of a particular byproduct compound of fermentation inhibits production of BIAs of interest when that compound has accumulated to a certain level. A modification that alleviates byproduct inhibition reduces the accumulation of one or more byproduct compounds in the engineered host cell relative to a control cell. In this way, the engineered host cell provides for a decreased level of the byproduct compound and/or an increased level of the BIAs of interest. By increased level is meant a level that is at least about 110% of that of the BIAs of interest in a control cell, such as about 120%, about 130%, about 140%, about 150%, about 160%, about 170%, about 180%, about 190%, about 200%, or more than 200%, such as at least about 3-fold, at least about 5-fold, at least about 10-fold of the BIAs of interest in the control cell. By decreased level is meant a level that is reduced by at least about about 10% or more, such as by about 20%, about 30%, about 40%, about 50%, about 60%, about 70%, about 80%, about 90%, about 95%, about 97%, or about 99% of that of the byproduct compound in a control cell. Modifications of host cell processes of interest that may be adapted for use in the subject host cells are described in U.S. Publication No. 20140273109 (Ser. No. 14/211,611) by Smolke et al., the disclosure of which is herein incorporated by reference in its entirety.

A variety of byproduct inhibition alleviating modifications and biosynthetic enzymes in the engineered host cell that are directed to modifying the accumulation of levels of byproducts of interest may be targeted for modification. The engineered host cell may include one or more byproduct inhibition alleviating mechanism in one or more biosynthetic enzyme genes. In some examples, the byproducts of interest are fusel alcohols. In some examples, the byproducts are tyrosol, phenylethanol, or methionol. In some examples, the engineered host cell may include one or more byproduct inhibition alleviating mechanisms in one or more biosynthetic enzyme genes such as one of those genes described in Table 11.

In some examples, the engineered host cell may include one or more heterologous coding sequences that encode one or more biosynthetic enzymes. In some examples, the biosynthetic enzymes are 4-hydroxyphenylacetaldehyde synthase (HPAAS). When HPAAS is expressed, it may convert L-tyrosine to 4-HPAA. In some examples, the biosynthetic enzymes are phosphoketolase (PK). When PK is expressed, it may convert fructose-6-phosphate and xylulose-5-phosphate to acetyl-phosphate. In some examples, the biosynthetic enzymes are uridine 5′-diphospho-glucosyltransferase (UGT). When UGT enzyme is expressed, it may convert a phenol to an aryl beta-D-glucose. In cases where UGT is expressed, it may be in combination with an inactivating mutation in EGH1 to increase the availability of the substrate UDP-glucose.

In some examples, the engineered host cell may include one or more inactivating mutations in one or more genes that encode biosynthetic enzymes. In some examples, the one or more inactivating mutations are in ARO8, ARO9, ARO10, PDC1, PDC5, PDC6, ARI1, ATF1, ATF2, EHT1, EEB1, AAD3, YPR1, GRE2, ADH1, ADH2, ADH3, ADH4, ADH5, ADH6, ADH7, YPR1, YDR541c BAT2, HFD1, TYR1, PHA2, DUG2, SFA1, or DUG3.

Any convenient numbers and types of modifications may be utilized to alleviate a byproduct inhibition mechanism. In certain embodiments, the engineered host cells of the present disclosure may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more byproduct inhibition alleviating modifications, such as 1, 2, 3, 4, 5, 6, 7, 8, 9, 10, 11, 12, 13, 14 or 15 byproduct inhibition alleviating modifications in one or more biosynthetic enzyme genes within the engineered host cell.

### Transcriptional Modulation Modifications

The host cells may include one or more transcriptional modulation modifications (such as two or more, three or more, four or more, five or more, or even more modifications) of one or more biosynthetic enzyme genes of the cell. In some examples, the one or more biosynthetic enzyme genes are native to the cell. In some examples, the one or more biosynthetic enzyme genes are non-native to the cell. Any convenient biosynthetic enzyme genes of the cell may be targeted for transcription modulation. By transcription modulation is meant that the expression of a gene of interest in a modified cell is modulated, e.g., increased or decreased, enhanced, or repressed, relative to a control cell (e.g., an unmodified cell). In some cases, transcriptional modulation of the gene of interest includes increasing or enhancing expression. By increasing or enhancing expression is meant that the expression level of the gene of interest is increased by 2-fold or more, such as by 5-fold or more and sometimes by 25-, 50-, or 100-fold or more and in certain embodiments 300-fold or more or higher, as compared to a control, i.e., expression in the same cell not modified (e.g., by using any convenient gene expression assay). Alternatively, in cases where expression of the gene of interest in a cell is so low that it is undetectable, the expression level of the gene of interest is considered to be increased if expression is increased to a level that is easily detectable. In certain instances, transcriptional modulation of the gene of interest includes decreasing or repressing expression. By decreasing or repressing expression is meant that the expression level of the gene of interest is decreased by 2-fold or more, such as by 5-fold or more and sometimes by 25-, 50-, or 100-fold or more and in certain embodiments 300-fold or more or higher, as compared to a control. In some cases, expression is decreased to a level that is undetectable. Modifications of host cell processes of interest that may be adapted for use in the subject host cells are described in U.S. Publication No. 20140273109 (Ser. No. 14/211,611) by Smolke et al., the disclosures of which are herein incorporated by reference in their entirety.

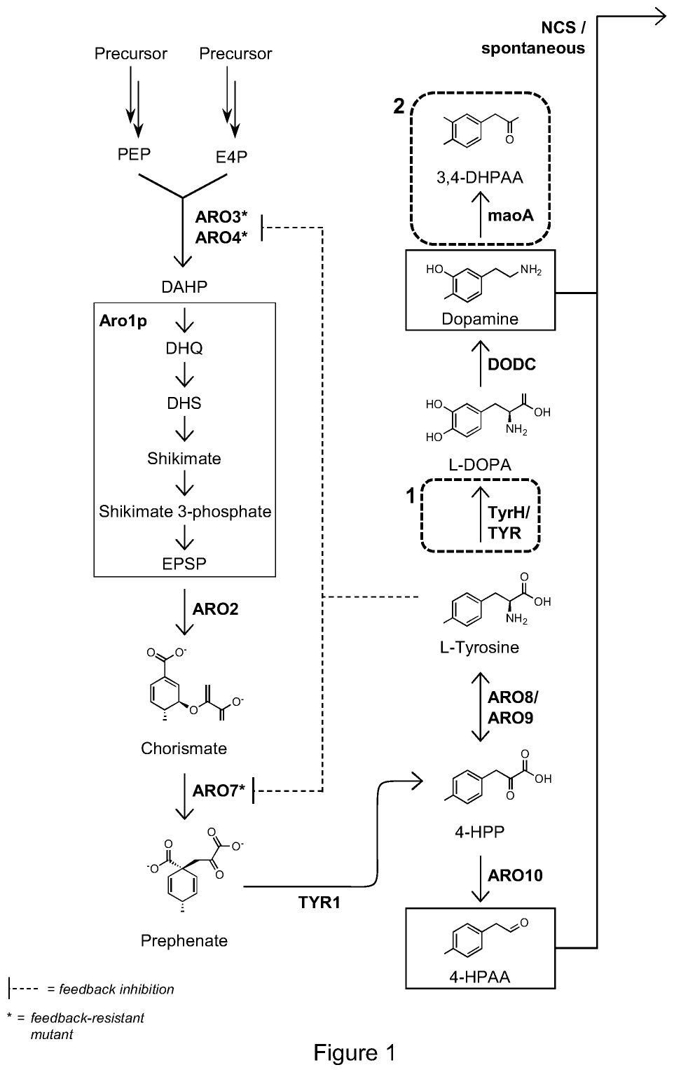

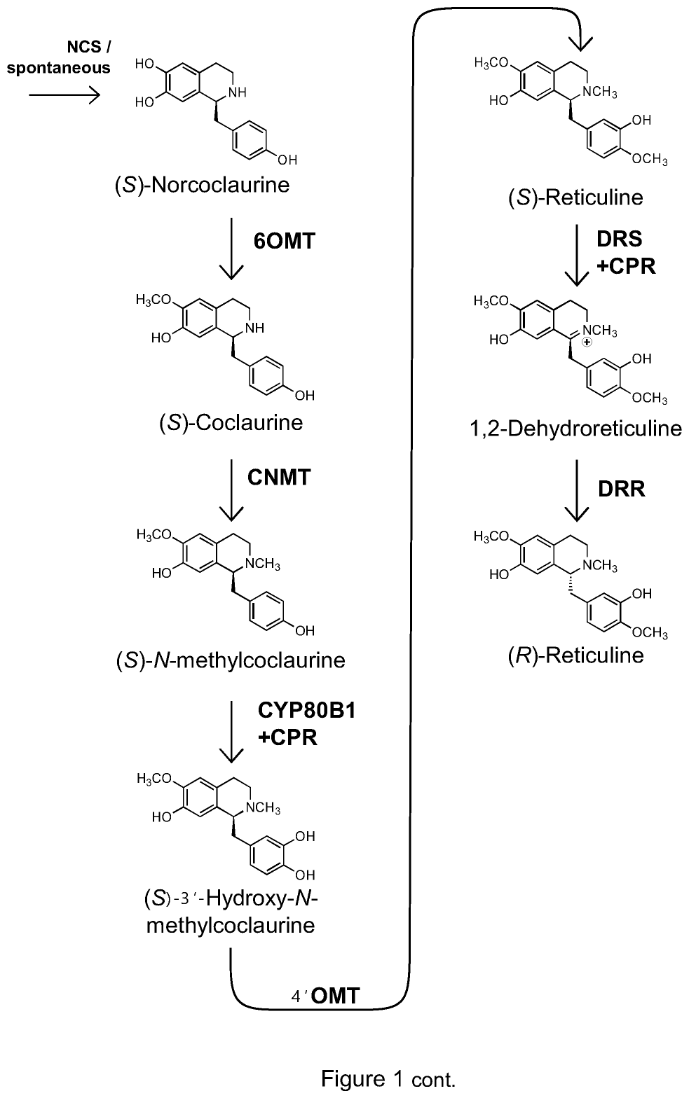

Any convenient biosynthetic enzyme genes may be transcriptionally modulated, and include but are not limited to, those biosynthetic enzymes described in FIG. 1. In particular, FIG. 1 illustrates a biosynthetic scheme for conversion of glucose to 4-HPAA, dopamine, and 3,4-DHPAA, in accordance with some embodiments of the disclosure. Examples of enzymes described in FIG. 1 include ARO3, ARO4, ARO1, ARO7, TYR1, TYR, TyrH, DODC, MAO, ARO10, ARO9, and ARO8. In some instances, the one or more biosynthetic enzyme genes may be selected from ARO10, ARO9, ARO8, and TYR1. In some cases, the one or more biosynthetic enzyme genes may be ARO10. In certain instances, the one or more biosynthetic enzyme genes may be ARO9. In some embodiments, the one or more biosynthetic enzyme genes may be TYR1. In some embodiments, the host cell includes one or more transcriptional modulation modifications to one or more genes such as one of those genes described in Table 11.

In some embodiments, the transcriptional modulation modification may include a substitution of a strong promoter for a native promoter of the one or more biosynthetic enzyme genes or the expression of an additional copy(ies) of the gene or genes under the control of a strong promoter. The promoters driving expression of the genes of interest may be constitutive promoters or inducible promoters, provided that the promoters may be active in the host cells. The genes of interest may be expressed from their native promoters. Additionally or alternatively, the genes of interest may be expressed from non-native promoters. Although not a requirement, such promoters may be medium to high strength in the host in which they are used. Promoters may be regulated or constitutive. In some embodiments, promoters that are not glucose repressed, or repressed only mildly by the presence of glucose in the culture medium, may be used. There are numerous suitable promoters, examples of which include promoters of glycolytic genes such as the promoter of the B. subtilis tsr gene (encoding fructose biphosphate aldolase) or GAPDH promoter from yeast S. cerevisiae (coding for glyceraldehyde-phosphate dehydrogenase) (Bitter G. A., Meth. Enzymol. 152:673 684 (1987)). Other strong promoters of interest include, but are not limited to, the ADHI promoter of baker's yeast (Ruohonen L., et al, J. Biotechnol. 39:193 203 (1995)), the phosphate-starvation induced promoters such as the PHO5 promoter of yeast (Hinnen, A., et al, in Yeast Genetic Engineering, Barr, P. J., et al. eds, Butterworths (1989), the alkaline phosphatase promoter from B. licheniformis (Lee. J. W. K., et al., J. Gen. Microbiol. 137:1127 1133 (1991)), GPD1, and TEF1. Yeast promoters of interest include, but are not limited to, inducible promoters such as Gal1-10, Gal1, GalL, GalS, repressible promoter Met25, tetO, and constitutive promoters such as glyceraldehyde 3-phosphate dehydrogenase promoter (GPD), alcohol dehydrogenase promoter (ADH), translation-elongation factor-1-alpha promoter (TEF), cytochrome c-oxidase promoter (CYC1), MRP7 promoter, etc. In some instances, the strong promoter is GPD1. In certain instances, the strong promoter is TEF1. Autonomously replicating yeast expression vectors containing promoters inducible by hormones such as glucocorticoids, steroids, and thyroid hormones are also known and include, but are not limited to, the glucorticoid responsive element (GRE) and thyroid hormone responsive element (TRE), see, e.g., those promoters described in U.S. Pat. No. 7,045,290. Vectors containing constitutive or inducible promoters such as alpha factor, alcohol oxidase, and PGH may be used. Additionally any convenient promoter/enhancer combination (as per the Eukaryotic Promoter Data Base EPDB) could also be used to drive expression of genes of interest. It is understood that any convenient promoters specific to the host cell may be selected, e.g., E. coli. In some cases, promoter selection may be used to optimize transcription, and hence, enzyme levels to maximize production while minimizing energy resources.

### Inactivating Mutations

The engineered host cells may include one or more inactivating mutations to an enzyme or protein of the cell (such as two or more, three or more, four or more, five or more, or even more). The inclusion of one or more inactivating mutations may modify the flux of a synthetic pathway of an engineered host cell to increase the levels of a BIA of interest or a desirable enzyme or precursor leading to the same. In some examples, the one or more inactivating mutations are to an enzyme native to the cell. Additionally or alternatively, the one or more inactivating mutations are to an enzyme non-native to the cell. As used herein, by “inactivating mutation” is meant one or more mutations to a gene or regulatory DNA sequence of the cell, where the mutation(s) inactivates a biological activity of the protein expressed by that gene of interest. In some cases, the gene is native to the cell. In some instances, the gene encodes an enzyme that is inactivated and is part of or connected to the synthetic pathway of a BIA of interest produced by the host cell. In some instances, an inactivating mutation is located in a regulatory DNA sequence that controls a gene of interest. In certain cases, the inactivating mutation is to a promoter of a gene. Any convenient mutations (e.g., as described herein) may be utilized to inactivate a gene or regulatory DNA sequence of interest. By “inactivated” or “inactivates” is meant that a biological activity of the protein expressed by the mutated gene is reduced by 10% or more, such as by 20% or more, 30% or more, 40% or more, 50% or more, 60% or more, 70% or more, 80% or more, 90% or more, 95% or more, 97% or more, or 99% or more, relative to a control protein expressed by a non-mutated control gene. In some cases, the protein is an enzyme, and the inactivating mutation reduces the activity of the enzyme.

In some examples, the engineered host cell includes an inactivating mutation in an enzyme or protein native to the cell. Any convenient enzymes may be targeted for inactivation. Enzymes of interest may include, but are not limited to those enzymes, described in Table 11 whose action in the synthetic pathway of the engineered host cell tends to reduce the levels of a BIA of interest. In some cases, the enzyme has glucose-6-phosphate dehydrogenase activity. In certain embodiments, the enzyme that includes an inactivating mutation is ZWF1. In some cases, the enzyme has alcohol dehydrogenase activity. In some embodiments, the enzyme that includes an inactivating mutation is selected from ADH2, ADH3, ADH4, ADH5, ADH6, ADH7, and SFA1. In certain embodiments, the enzyme that includes an inactivating mutation(s) is ADH2. In certain embodiments, the enzyme that includes an inactivating mutation(s) is ADH3. In certain embodiments, the enzyme that includes an inactivating mutation(s) is ADH4. In certain embodiments, the enzyme that includes an inactivating mutation(s) is ADH5. In certain embodiments, the enzyme that includes an inactivating mutation(s) is ADH6. In certain embodiments, the enzyme that includes an inactivating mutation(s) is ADH7. In some cases, the enzyme has aldehyde oxidoreductase activity. In certain embodiments, the enzyme that includes an inactivating mutation is selected from ALD2, ALD3, ALD4, ALD5, and ALD6. In certain embodiments, the enzyme that includes an inactivating mutation(s) is ALD2. In certain embodiments, the enzyme that includes an inactivating mutation(s) is ALD3. In certain embodiments, the enzyme that includes an inactivating mutation(s) is ALD4. In certain embodiments, the enzyme that includes an inactivating mutation(s) is ALD5. In certain embodiments, the enzyme that includes an inactivating mutation(s) is ALD6. In some cases, the enzyme has aldehyde reductase activity. In some embodiments, the enzyme that includes an inactivating mutation is ARI1. In some cases, the enzyme has aryl-alcohol dehydrogenase activity. In some embodiments, the enzyme that includes an inactivating mutation is selected from AAD4, AAD6, AAD10, AAD14, AAD15, AAD16. In certain embodiments, the enzyme that includes an inactivating mutation(s) is AAD4. In certain embodiments, the enzyme that includes an inactivating mutation(s) is AAD6. In certain embodiments, the enzyme that includes an inactivating mutation(s) is AAD10. In certain embodiments, the enzyme that includes an inactivating mutation(s) is AAD14. In certain embodiments, the enzyme that includes an inactivating mutation(s) is AAD15. In certain embodiments, the enzyme that includes an inactivating mutation(s) is AAD16. In some examples, the engineered host cell includes an inactivating mutation in a transcription regulator native to the cell. Transcriptional regulators of interest may include, but are not limited to those proteins, described in Table 11. In some cases, the protein has activity as a transcriptional regulator of phospholipid biosynthetic genes. In some embodiments, the transcriptional regulator that includes an inactivating mutation is OPI1. In some embodiments, the host cell includes one or more inactivating mutations to one or more genes described in Table 11.

In some examples, the engineered host cell includes an inactivating mutation in an enzyme or protein native to the cell. Enzymes of interest may include, but are not limited to those enzymes, described in Table 11 whose action in the synthetic pathway of the engineered host cell is part of the Erlich pathway to produce fusel alcohols. In some cases, the enzyme has phenylpyruvate decarboxylase activity. In certain embodiments, the enzyme that includes an inactivating mutation is ARO10. In some cases, the enzyme has pyruvate decarboxylase activity. In some embodiments, the enzyme that includes an inactivating mutation is selected from PDC1, PDC5, and PDC6. In certain embodiments, the enzyme that includes an inactivating mutation(s) is PDC1. In certain embodiments, the enzyme that includes an inactivating mutation(s) is PDC5. In certain embodiments, the enzyme that includes an inactivating mutation(s) is PDC6. In some cases, the enzyme has aromatic aminotransferase activity. In some embodiments, the enzyme that includes an inactivating mutation is selected from ARO8 and ARO9. In certain embodiments, the enzyme that includes an inactivating mutation(s) is ARO8. In certain embodiments, the enzyme that includes an inactivating mutation(s) is ARO9. In some cases, the enzyme has prephenate dehydrogenase activity. In certain embodiments, the enzyme that includes an inactivating mutation(s) is TYR1. In some cases, the enzyme has prephenate dehydratase activity. In certain embodiments, the enzyme that includes an inactivating mutation(s) is PHA2. In some embodiments, the host cell includes one or more inactivating mutations to one or more genes described in Table 11.

### Epimerization Modifications

Some methods, processes, and systems provided herein describe the conversion of (S)-1-benzylisoquinoline alkaloids to (R)-1-benzylisoquinoline alkaloids. Some of these methods, processes, and systems may comprise an engineered host cell. In some examples, the conversion of (S)-1-benzylisoquinoline alkaloids to (R)-1-benzylisoquinoline alkaloids is a key step in the conversion of a substrate to a diverse range of alkaloids. In some examples, the conversion of (S)-1-benzylisoquinoline alkaloids to (R)-1-benzylisoquinoline alkaloids comprises an epimerization reaction via an engineered epimerase. In some cases, epimerization of a substrate alkaloid may be performed by oxidizing an (S)-substrate to the corresponding Schiff base or imine intermediate, then stereospecifically reducing this intermediate to an (R)-product as provided in FIG. 1 and as represented generally in Scheme 1. As provided in Scheme 1, R₁, R₂, R₃, and R₄ may be H or CH₃. R₅ may be H, OH, or OCH₃.

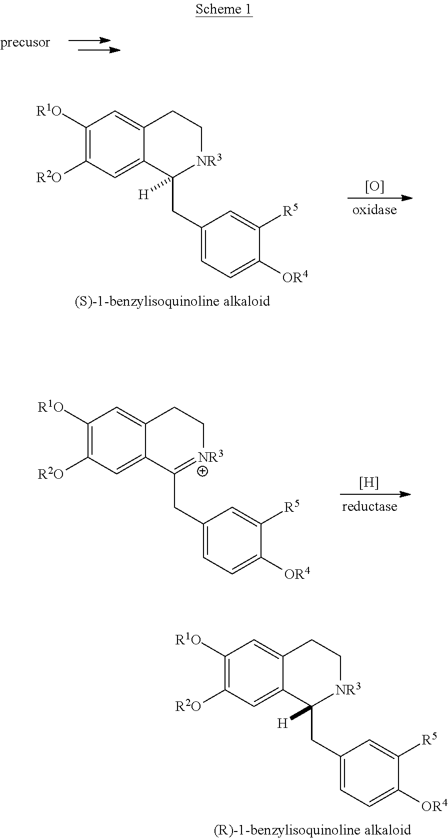

In some examples, the conversion of the (S)-substrate to the (R)-product may involve at least one oxidation reaction and at least one reduction reaction. In some cases, an oxidation reaction is optionally followed by a reduction reaction. In some cases, at least one of the oxidation and reduction reactions is carried out in the presence of an enzyme. In some cases, at least one of the oxidation and reduction reactions is catalyzed by an engineered epimerase. In some cases, the oxidation and reduction reactions are both carried out in the presence of an engineered fused epimerase. In some cases, the oxidation and reduction reactions are both carried out in the presence of an engineered split epimerase having a separately expressed oxidase component and reductase component, respectively. In some cases, an engineered epimerase is useful to catalyze the oxidation and reduction reactions. The oxidation and reduction reactions may be catalyzed by the same engineered epimerase.

In some methods, processes and systems described herein, an oxidation reaction may be performed in the presence of an enzyme that is part of an engineered epimerase. In some examples, the engineered epimerase may have an oxidase component. In some cases, the oxidase component may be a component of an engineered fused epimerase. In some case, the oxidase component may be independently expressed as part of an engineered split epimerase. The oxidase may use a (S)-1-benzylisoquinoline as a substrate. The oxidase may convert the (S)-substrate to a corresponding imine or Schiff base derivative. The oxidase may be referred to as 1,2-dehydroreticuline synthase (DRS). Non-limiting examples of enzymes suitable for oxidation of (S)-1-benzylisoquinoline alkaloids in this disclosure include a cytochrome P450 oxidase, a 2-oxoglutarate-dependent oxidase, and a flavoprotein oxidase. For example, (S)-tetrahydroprotoberberine oxidase (STOX, E.C 1.3.3.8) may oxidize (S)-norreticuline and other (S)-1-benzylisoquinoline alkaloids to 1,2-dehydronorreticuline and other corresponding 1,2-dehydro products. In some examples, a protein that comprises an oxidase domain of any one of the preceding examples may perform the oxidation. In some examples, the oxidase may catalyze the oxidation reaction within a host cell, such as an engineered host cell, as described herein. In some cases, the oxidase may have one or more activity-increasing components. In some examples, a reduction reaction may follow the oxidation reaction. The reduction reaction may be performed by an enzyme that is part of an engineered epimerase. In some examples, the reductase may use an imine or Schiff base derived from a 1-benzylisoquinoline as a substrate. The reductase may convert the imine or Schiff base derivative to a (R)-1-benzylisoquinoline. The reductase may be referred to as 1,2-dehydroreticuline reductase (DRR). Non-limiting examples of enzymes suitable for reduction of an imine or Schiff base derived from an (S)-1-benzylisoquinoline alkaloid include an aldo-keto reductase (e.g., a codeinone reductase-like enzyme (EC 1.1.1.247)) and a short chain dehydrogenase (e.g., a salutaridine reductase-like enzyme (EC 1.1.1.248)). In some examples, a protein that comprises a reductase domain of any one of the preceding examples may perform the reduction. In a further embodiment, the reduction is stereospecific. In some examples, the reductase may catalyze the reduction reaction within a host cell, such as an engineered host cell, as described herein.

An example of an enzyme that can perform an epimerization reaction that converts (S)-1-benzylisoquinoline alkaloids to (R)-1-benzylisoquinoline alkaloids includes an epimerase having an oxidase domain and a reductase domain. In particular, the epimerase may have a cytochrome P450 oxidase 82Y2-like domain. Additionally, the epimerase may have a codeinone reductase-like domain. An epimerase having a cytochrome P450 oxidase 82Y2-like domain and also having a codeinone reductase-like domain may be referred to as a DRS-DRR enzyme. In particular, a DRS-DRR enzyme may be a fusion enzyme that is a fusion epimerase. Further, when a DRS-DRR enzyme is modified by at least one activity-increasing modification, the fusion enzyme may be an engineered fusion epimerase.

Examples of amino acid sequences of a DRS-DRR enzyme that may be used to perform the conversion of (S)-1-benzylisoquinoline alkaloids to (R)-1-benzylisoquinoline alkaloids are set forth in Table 1. An amino acid sequence for an epimerase that is utilized in converting an (S)-1-benzylisoquinoline alkaloid to an (R)-1-benzylisoquinoline alkaloid may be 50% or more identical to a given amino acid sequence as listed in Table 1. For example, an amino acid sequence for such an epimerase may comprise an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the nucleic acid sequence encoding the amino acid sequence is altered such as to optimize codon usage for the host organism, for example.

Amino acid residues of homologous epimerases may be referenced according to the numbering scheme of SEQ ID NO. 16, and this numbering system is used throughout the disclosure to refer to specific amino acid residues of epimerases which are homologous to SEQ ID NO. 16. Epimerases homologous to SEQ ID NO. 16 may have at least about 50%, 55%, 60%, 65%, 70%, 75%, 80%, 85%, 90%, 95%, 97%, 98%, or 99% sequence identity to SEQ ID NO. 16. In some cases, an amino acid referred to as position 50 in a homologous epimerase may not be the 50^{th} amino acid in the homologous epimerase, but would be the amino acid which corresponds to the amino acid at position 50 in SEQ ID NO. 16 in a protein alignment of the homologous epimerase with SEQ ID NO. 16. In some cases, homologous enzymes may be aligned with SEQ ID NO. 16 either according to primary sequence, secondary structure, or tertiary structure.

An engineered host cell may be provided that produces an engineered epimerase that converts (S)-1-benzylisoquinoline alkaloid to (R)-1-benzylisoquinoline alkaloid, wherein the epimerase comprises an amino acid sequence selected from the group consisting of: SEQ ID NOs: 1, 2, 3, 4, 5, 6, 7, 8, 9, 10, 11, 12, 13, 14, 15, 16, 17, and 18, and having one or more activity-enhancing modifications. The epimerase that is produced within the engineered host cell may be recovered and purified so as to form a biocatalyst. In some cases, the epimerase may be split into one or more enzymes. Additionally, one or more enzymes that are produced by splitting the epimerase may be recovered from the engineered host cell. These one or more enzymes that result from splitting the epimerase may also be used to catalyze the conversion of (S)-1-benzylisoquinoline alkaloids to (R)-1-benzylisoquinoline alkaloids. Additionally, the use of an engineered split epimerase may be used to increase the production of benzylisoquinoline alkaloid products within a cell when compared to the production of benzylisoquinoline alkaloid products within a cell utilizing a fused epimerase.

In additional cases, the one or more enzymes that are recovered from the engineered host cell that produces the epimerase may be used in a process for converting a (S)-1-benzylisoquinoline alkaloid to a (R)-1-benzylisoquinoline alkaloid. The process may include contacting the (S)-1-benzylisoquinoline alkaloid with an epimerase in an amount sufficient to convert said (S)-1-benzylisoquinoline alkaloid to (R)-1-benzylisoquinoline alkaloid. In some examples, the (S)-1-benzylisoquinoline alkaloid may be contacted with a sufficient amount of the one or more enzymes such that at least 5% of said (S)-1-benzylisoquinoline alkaloid is converted to (R)-1-benzylisoquinoline alkaloid. In further examples, the (S)-1-benzylisoquinoline alkaloid may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said (S)-1-benzylisoquinoline alkaloid is converted to (R)-1-benzylisoquinoline alkaloid.

The one or more enzymes that may be used to convert a (S)-1-benzylisoquinoline alkaloid to a (R)-1-benzylisoquinoline alkaloid may contact the (S)-1-benzylisoquinoline alkaloid in vitro. Additionally, or alternatively, the one or more enzymes that may be used to convert a (S)-1-benzylisoquinoline alkaloid to a (R)-1-benzylisoquinoline alkaloid may contact the (S)-1-benzylisoquinoline alkaloid in vivo. Additionally, the one or more enzymes that may be used to convert a (S)-1-benzylisoquinoline alkaloid to a (R)-1-benzylisoquinoline alkaloid may be provided to a cell having the (S)-1-benzylisoquinoline alkaloid within, or may be produced within an engineered host cell.

In some examples, the methods provide for engineered host cells that produce an alkaloid product, wherein the epimerization of a (S)-substrate to a (R)-product may comprise a key step in the production of an alkaloid product. In some examples, the alkaloid produced is a (R)-1-benzylisoquinoline alkaloid. In still other embodiments, the alkaloid produced is derived from a (R)-1-benzylisoquinoline alkaloid, including, for example, 4-ring promorphinan and 5-ring morphinan alkaloids. In another embodiment, a (S)-1-benzylisoquinoline alkaloid is an intermediate toward the product of the engineered host cell. In still other embodiments, the alkaloid product is selected from the group consisting of 1-benzylisoquinoline, morphinan, promorphinan, nor-opioid, nal-opioid, or bisbenzylisoquinoline alkaloids.

In some examples, the (S)-substrate is a (S)-1-benzylisoquinoline alkaloid selected from the group consisting of (S)-norreticuline, (S)-reticuline, (S)-tetrahydropapaverine, (S)-norcoclaurine, (S)-coclaurine, (S)—N-methylcoclaurine, (S)-3′-hydroxy-N-methylcoclaurine, (S)-norisoorientaline, (S)-orientaline, (S)-isoorientaline, (S)-norprotosinomenine, (S)-protosinomenine, (S)-norlaudanosoline, (S)-laudanosoline, (S)-4′-O-methyllaudanosoline, (S)-6-O-methylnorlaudanosoline, (S)-4′-O-methylnorlaudanosoline.

In some examples, the (S)-substrate is a compound of Formula I:

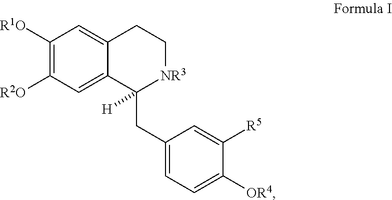

or a salt thereof, wherein:
R¹, R², R³, and R⁴ are independently selected from hydrogen and methyl; and
R⁵ is selected from hydrogen, hydroxy, and methoxy.

In some other examples, at least one of R¹, R², R³, R⁴, and R⁵ is hydrogen.

In still other examples, the (S)-substrate is a compound of Formula II:

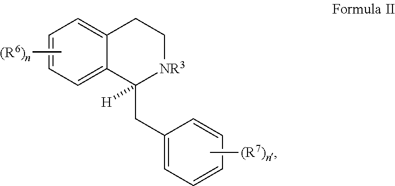

or a salt thereof, wherein:
R³ is selected from hydrogen and C₁-C₄ alkyl;
R⁶ and R⁷ are independently selected at each occurrence from hydroxy, fluoro, chloro, bromo, carboxaldehyde, C₁-C₄ acyl, C₁-C₄ alkyl, and C₁-C₄ alkoxy;
n is 0, 1, 2, 3, or 4; and
n′ is 0, 1, 2, 3, 4 or 5.

When a bond is drawn across a ring, it means substitution may occur at a non-specific ring atom or position. For example, in Formula II shown above, the hydrogen of any —CH— in the 6-membered ring may be replaced with R⁷ to form —CR⁷—.

In some examples, R⁶ and R⁷ are independently methyl or methoxy. In some other examples, n and n′ are independently 1 or 2. In still other embodiments, R is hydrogen or methyl.

In some examples, the methods provide for engineered host cells that produce alkaloid products from (S)-reticuline. The epimerization of (S)-reticuline to (R)-reticuline may comprise a key step in the production of diverse alkaloid products from a precursor. In some examples, the precursor is L-tyrosine or a sugar (e.g., glucose). The diverse alkaloid products can include, without limitation, 1-benzylisoquinoline, morphinan, promorphinan, nor-opioid, or nal-opioid alkaloids.

Any suitable carbon source may be used as a precursor toward an epimerized 1-benzylisoquinoline alkaloid. Suitable precursors can include, without limitation, monosaccharides (e.g., glucose, fructose, galactose, xylose), oligosaccharides (e.g., lactose, sucrose, raffinose), polysaccharides (e.g., starch, cellulose), or a combination thereof. In some examples, unpurified mixtures from renewable feedstocks can be used (e.g., cornsteep liquor, sugar beet molasses, barley malt, biomass hydrolysate). In still other embodiments, the carbon precursor can be a one-carbon compound (e.g., methanol, carbon dioxide) or a two-carbon compound (e.g., ethanol). In yet other embodiments, other carbon-containing compounds can be utilized, for example, methylamine, glucosamine, and amino acids (e.g., L-tyrosine). In some examples, a 1-benzylisoquinoline alkaloid may be added directly to an engineered host cell of the disclosure, including, for example, norlaudanosoline, laudanosoline, norreticuline, and reticuline. In still further embodiments, a 1-benzylisoquinoline alkaloid may be added to the engineered host cell as a single enantiomer (e.g., a (S)-1-benzylisoquinoline alkaloid), or a mixture of enantiomers, including, for example, a racemic mixture.

In some examples, the methods provide for the epimerization of a stereocenter of a 1-benzylisoquinoline alkaloid, or a derivative thereof, using an engineered epimerase. In a further embodiment, the method comprises contacting the 1-benzylisoquinoline alkaloid with an engineered epimerase. The engineered epimerase may invert the stereochemistry of a stereocenter of a 1-benzylisoquinoline alkaloid, or derivative thereof, to the opposite stereochemistry. In some examples, the engineered epimerase converts a (S)-1-benzylisoquinoline alkaloid to a (R)-1-benzylisoquinoline alkaloid. In some examples of this conversion of a (S)-1-benzylisoquinoline alkaloid to a (R)-1-benzylisoquinoline alkaloid utilizing the engineered epimerase, the (S)-1-benzylisoquinoline alkaloid is selected from the group consisting of (S)-norreticuline, (S)-reticuline, (S)-tetrahydropapaverine, (S)-norcoclaurine, (S)-coclaurine, (S)—N-methylcoclaurine, (S)-3′-hydroxy-N-methylcoclaurine, (S)-norisoorientaline, (S)-orientaline, (S)-isoorientaline, (S)-norprotosinomenine, (S)-protosinomenine, (S)-norlaudanosoline, (S)-laudanosoline, (S)-4′-O-methyllaudanosoline, (S)-6-O-methylnorlaudanosoline, and (S)-4′-O-methylnorlaudanosoline.

In still other embodiments, the 1-benzylisoquinoline alkaloid that is epimerized using an engineered epimerase may comprise two or more stereocenters, wherein only one of the two or more stereocenters is inverted to produce a diastereomer of the substrate (e.g., (S, R)-1-benzylisoquinoline alkaloid converted to (R, R)-1-benzylisoquinoline alkaloid). In some examples where only one stereocenter of a 1-benzylisoquinoline alkaloid is inverted when contacted with the at least one enzyme, the product is referred to as an epimer of the 1-benzylisoquinoline alkaloid.

In some examples, the 1-benzylisoquinoline alkaloid is presented to the enzyme as a single stereoisomer. In some other examples, the 1-benzylisoquinoline alkaloid is presented to the enzyme as a mixture of stereoisomers. In still further embodiments, the mixture of stereoisomers may be a racemic mixture. In some other examples, the mixture of stereoisomers may be enriched in one stereoisomer as compared to another stereoisomer.

In some examples, a 1-benzylisoquinoline alkaloid, or a derivative thereof, is recovered. In some examples, the 1-benzylisoquinoline alkaloid is recovered from a cell culture. In still further embodiments, the recovered 1-benzylisoquinoline alkaloid is enantiomerically enriched in one stereoisomer as compared to the original mixture of 1-benzylisoquinoline alkaloids presented to the enzyme. In still further embodiments, the recovered 1-benzylisoquinoline alkaloid has an enantiomeric excess of at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100%.

In some examples, a promorphinan, or a derivative thereof, is recovered. In some examples, the promorphinan is recovered from a cell culture. In still further embodiments, the recovered promorphinan has an enantiomeric excess of at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100%.

In some examples, a morphinan, or a derivative thereof, is recovered. In some examples, the morphinan is recovered from a cell culture. In still further embodiments, the recovered morphinan has an enantiomeric excess of at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100%.

In some examples, a bisbenzylisoquinoline, or a derivative thereof, is recovered. In some examples, the bisbenzylisoquinoline is recovered from a cell culture. In still further embodiments, the recovered bisbenzylisoquinoline is enantiomerically enriched in one stereoisomer as compared to the original mixture of bisbenzylisoquinoline presented to the enzyme. In still further embodiments, the recovered bisbenzylisoquinoline has an enantiomeric excess of at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100%.

In some examples, a nal-opioid, or a derivative thereof, is recovered. In some examples, the nal-opioid is recovered from a cell culture. In still further embodiments, the recovered nal-opioid has an enantiomeric excess of at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100%.

In some examples, a nor-opioid, or a derivative thereof, is recovered. In some examples, the nor-opioid is recovered from a cell culture. In still further embodiments, the recovered nor-opioid has an enantiomeric excess of at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100%.

“Isomers” are different compounds that have the same molecular formula. “Stereoisomers” are isomers that differ only in the way the atoms are arranged in space. “Enantiomers” are a pair of stereoisomers that are non superimposable mirror images of each other. A 1:1 mixture of a pair of enantiomers is a “racemic” mixture. “Diastereoisomers” or “diastereomers” are stereoisomers that have at least two asymmetric atoms but are not mirror images of each other. The term “epimer” as used herein refers to a compound having the identical chemical formula but a different optical configuration at a particular position. For example, the (R,S) and (S,S) stereoisomers of a compound are epimers of one another. In some examples, a 1-benzylisoquinoline alkaloid is converted to its epimer (e.g., epi-1-benzylisoquinoline alkaloid). The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms that can be defined, in terms of absolute stereochemistry, as (R)- or (S)-.

TABLE 1
Example amino acid sequences of DRS-DRR enzymes,
split DRS and DRR enzymes, and other nucleotide sequences

| | | SEQ. ID |
|---|---|---|
| Sequence | Description | NO. |
| MELQYISYFQPTSSVVALLLALVSILSSVVVLRKTFLNNYSSSPASSTKTAVLSH | P. | SEQ. ID |
| QRQQSCALPISGLLHIFMNKNGLIHVTLGNMADKYGPIFSFPTGSHRTLVVSSWE | somniferum | NO. 1 |
| MVKECFTGNNDTAFSNRPIPLAFKTIFYACGGIDSYGLSSVPYGKYWRELRKVC | plant source; | |
| VHNLLSNQQLLKFRHLIISQVDTSFNKLYELCKNSEDNHGNYTTTTTTAAGMVR | full-length | |
| IDDWLAELSFNVIGRIVCGFQSGPKTGAPSRVEQFKEAINEASYFMSTSPVSDNV | amino acid | |
| PMLGWIDQLTGLTRNMKHCGKKLDLVVESIINDHRQKRRFSRTKGGDEKDDEQ | sequence | |
| DDFIDICLSIMEQPQLPGNNNPSQIPIKSIVLDMIGGGTDTTKLTTIWTLSLLLNNP | >RQNK- | |
| HVLDKAKQEVDAHFRTKRRSTNDAAAAVVDFDDIRNLVYIQAIIKESMRLYPA | 2062398 | |
| SPVVERLSGEDCVVGGFHVPAGTRLWANVWKMQRDPKVWDDPLVFRPDRFLS | (also FPYZ- | |
| DEQKMVDVRGQNYELLPFGAGRRVCPGVSFSLDLMQLVLTRLILEFEMKSPSG | 2037562, | |
| KVDMTATPGLMSYKVIPLDILLTHRRIKPCVQSAASERDMESSGVPVITLGSGK | BMRX- | |
| VMPVLGMGTFEKVGKGSERERLAILKAIEVGYRYFDTAAAYETEEVLGEAIAE | 2007040, and | |
| ALQLGLVKSRDELFISSMLWCTDAHADRVLLALQNSLRNLKLEYVDLYMLPFP | MLPX- | |
| ASLKPGKITMDIPEEDICRMDYRSVWAAMEECQNLGFTKSIGVSNFSCKKLQEL | 2016197) | |
| MATANIPPAVNQVEMSPAFQQKKLREYCNANNILVSAISVLGSNGTPWGSNAV | | |
| LGSEVLKKIAMAKGKSVAQVSMRWVYEQGASLVVKSFSEERLRENLNIFDWEL | | |
| TKEDHEKIGEIPQCRILSAYFLVSPNGPFKSQEELWDDEA* | | |
| MELQYISYFQPTSSVVALLLALVSILSSVVVLRKTFLNNYSSSPASSTKTAVLSH | P. | SEQ. ID |
| QRQQSCALPISGLLHIFMNKNGLIHVTLGNMADKYGPIFSFPTGSHRTLVVSSWE | somniferum | NO. 2 |
| MVKECFTGNNDTAFSNRPIPLAFKTIFYACGGIDSYGLSSVPYGKYWRELRKVC | plant source; | |
| VHNLLSNQQLLKFRHLIISQVDTSFNKLYELCKNSEDNHGNYTTXLLLPQLAWR | full-length | |
| QPWKLYYXTTTTAAGMVRIDDWLAELSFNVIGRIVCGFQSGPKTGAPSRVEQF | amino acid | |
| KEAINEASYFMSTSPVSDNVPMLGWIDQLTGLTRNMKHCGKKLDLVVESIIND | sequence | |
| HRQKRRFSRTKGGDEKDDEQDDFIDICLSIMEQPQLPGNNNPSQIPIKSIVLDMIG | >KKCW- | |
| GGTDTTKLTTIWTLSLLLNNPHVLDKAKQEVDAHFRTKRRSTNDAAAAVVDFD | 2026866 | |
| DIRNLVYIQAIIKESMRLYPASPVVERLSGEDCVVGGFHVPAGTRLWANVWKM | (also FPYZ- | |
| QRDPKVWDDPLVFRPDRFLSDEQKMVDVRGQNYELLPFGAGRRVCPGVSFSL | 2037562, | |
| DLMQLVLTRLILEFEMKSPSGKVDMTATPGLMSYKVIPLDILLTHRRIKPCVQSA | MLPX- | |
| ASERDMESSGVPVITLGSGKVMPVLGMGTFEKVGKGSERERLAILKAIEVGYRY | 2016197) | |
| FDTAAAYETEEVLGEAIAEALQLGLVKSRDELFISSMLWCTDAHADRVLLALQ | | |
| NSLRNLKLEYVDLYMLPFPASLKPGKITMDIPEEDICRMDYRSVWAAMEECQN | | |
| LGFTKSIGVSNFSCKKLQELMATANIPPAVNQVEMSPAFQQKKLREYCNANNIL | | |
| VSAISVLGSNGTPWGSNAVLGSEVLKKIAMAKGKSVAQVSMRWVYEQGASLV | | |
| VKSFSEERLRENLNIFDWELTKEDHEKIGEIPQCRILSAYFLVSPNGPFKSQEELW | | |
| DDEA* | | |
| MELQYISYFQPTSSVVALLLALVSILSSVVVLRKTFLNNYSSSPASSTKTAVLSH | P. | SEQ. ID |
| QRQQSCALPISGLLHIFMNKNGLIHVTLGNMADKYGPIFSFPTGSHRTLVVSSWE | somniferum | NO. 3 |
| MVKECFTGNNDTAFSNRPIPLAFKTIFYACGGIDSYGLSSVPYGKYWRELRKVC | plant source; | |
| VHNLLSNQQLLKFRHLIISQVDTSFNKLYELCKNSEDNHGNYTTTTTTAAGMVR | partial-length | |
| IDDWLAELSFNVIGRIVCGFQSGPKTGAPSRVEQFKEAINEASYFMSTSPVSDNV | amino acid | |
| PMLGWIDQLTGLTRNMKHCGKKLDLVVESIINDHRQKRRFSRTKGGDEKDDEQ | sequence | |
| DDFIDICLSIMEQPQLPGNNNPSQIPIKSIVLDMIGGGTDTTKLTTIWTLSLLLNNP | >SUFP- | |
| HVLDKAKQEVDAHFRTKRRSTNDAAAAVVDFDDIRNLVYIQAIIKESMRLYPA | 2025636 | |
| SPVVERLSGEDCVVGGFHVPAGTRLWANVWKMQRDPKVWDDPLVFRPDRFLS | | |
| DEQKMVDVRGQNYELLPFGAGRRVCPGVSFSLDLMQLVLTRLILEFEMKSPSG | | |
| KVDMTATPGLMSYKVIPLDILLTHRRIKPCVQSAASERDMESSGVPVITLGSGK | | |
| VMPVLGMGTFEKVGKGSERERLAILKAIEVGYRYFDTAAAYETEEVLGEAIAE | | |
| ALQLGLVKSRDELFISSMLWCTDAHADRVLLALQNSLRNLKLEYVDLYMLPFP | | |
| ASLKPGKITMDIPEEDICRMDYRXVSKPWLH* | | |
| MRWHRXIDSYGLSSVPYGKYWRELRKVCVHNLLSNQQLLKFRHLIISQVDTSF | P. | SEQ. ID |
| NKLYELCKNSEDNQGNYPTTTTAAGMVRIDDWLAELSFNVIGRIVCGFQSGPK | somniferum | NO. 4 |
| TGAPSRVEQFKEAINEASYFMSTSPVSDNVPMLGWIDQLTGLTRNMKHCGKKL | plant source; | |
| DLVVESIINDHRQKRRFSRTKGGDEKDDEQDDFIDICLSIMEQPQLPGNNNPSQIP | partial-length | |
| IKSIVLDMIGGGTDTTKLTTIWTLSLLLNNPHVLDKAKQEVDAHFRTKRRSTND | amino acid | |
| AAAAVVDFDDIRNLVYIQAIIKESMRLYPASPVVERLSGEDCVVGGFHVPAGTR | sequence | |
| LWANVWKMQRDPKVWDDPLVFRPDRFLSDEQKMVDVRGQNYELLPFGAGRR | >MIKW- | |
| VCPGVSFSLDLMQLVLTRLILEFEMKSPSGKVDMTATPGLMSYKVIPLDILLTHR | 2013651 | |
| RIKPCVQSAASERDMESSGVPVITLGSGKVMPVLGMGTFEKVGKGSERERLAIL | | |
| KAIEVGYRYFDTAAAYETEEVLGEAIAEALQLGLVKSRDELFISSMLWCTDAHA | | |
| DRVLLALQNSLRNLKLEYVDLYMLPFPASLKPGKITMDIPEEDICRMDYRSVW | | |
| AAMEECQNLGFTKSIGVSNFSCKKLQELMATANIPPAVNQVEMSPAFQQKKLR | | |
| EYCNANNILVSAISVLGSNGTPWGSNAVLGSEVLKKIAMAKGKSVAQVSMRW | | |
| VYEQGASLVVKSFSEERLRENLNIFDWELTKEDHEKIGEIPQCRILSAYFLVSPN | | |
| GPFKSQEELWDDEA* | | |
| MELQYISYFQPTSSVVALLLALVSILSSVVVLRKTFLNNYSSSPASSTKTAVLSH | P. setigerum | SEQ. ID |
| QRQQSCALPISGLLHIFMNKNGLIHVTLGNMADKYGPIFSFPTGSHRTLVVSSWE | plant source; | NO. 5 |
| MVKECFTGNNDTAFSNRPIPLAFKTIFYACGGIDSYGLSSVPYGKYWRELRKVC | full-length | |
| VHNLLSNQQLLKFRHLIISQVDTSFNKLYELCKNSEDNQGNYTTTTTAAGMVRI | amino acid | |
| DDWLAELSFNVIGRIVCGFQSGPKTGAPSRVEQFKEAINEASYFMSTSPVSDNVP | sequence | |
| MLGWIDQLTGLTRNMKHCGKKLDLVVESIINDHRQKRRFSRTKGGDEKDDEQ | >EPRK- | |
| DDFIDICLSIMEQPQLPGNNNPSQIPIKSIVLDMIGGGTDTTKLTTIWTLSLLLNNP | 2027940 | |
| HVLDKAKQEVDAHFRTKRRSTNDAAAAVVDFDDIRNLVYIQAIIKESMRLYPA | (also FPYZ- | |
| SPVVERLSGEDCVVGGFHVPAGTRLWANVWKMQRDPKVWDDPLVFRPDRFLS | 2037562, | |
| DEQKMVDVRGQNYELLPFGAGRRVCPGVSFSLDLMQLVLTRLILEFEMKSPSG | STDO- | |
| KVDMTATPGLMSYKVIPLDILLTHRRIKPCVQSAASERDMESSGVPVITLGSGK | 2019715, | |
| VMPVLGMGTFEKVGKGSERERLAILKAIEVGYRYFDTAAAYETEEVLGEAIAE | FNXH- | |
| ALQLGLVKSRDELFISSMLWCTDAHADRVLLALQNSLRNLKLEYVDLYMLPFP | 2029312, | |
| ASLKPGKITMDIPEEDICRMDYRSVWAAMEECQNLGFTKSIGVSNFSCKKLQEL | MLPX- | |
| MATANIPPAVNQVEMSPAFQQKKLREYCNANNILVSAISVLGSNGTPWGSNAV | 2016196, | |
| LGSEVLKKIAMAKGKSVAQVSMRWVYEQGASLVVKSFSEERLRENLNIFDWEL | MLPX- | |
| TKEDHEKIGEIPQCRILSAYFLVSPNGPFKSQEELWDDEA* | 2016197) | |
| MELQYISYFQPTSSVVALLLALVSILSSVVVLRKTFLNNYSSSPASSTKTAVLSH | P. setigerum | SEQ. ID |
| QRQQSCALPISGLLHIFMNKNGLIHVTLGNMADKYGPIFSFPTGSHRTLVVSSWE | plant source; | NO. 6 |
| MVKECFTGNNDTAFSNRPIPLAFKTIFYACGGIDSYGLSSVPYGKYWRELRKVC | partial-length | |
| VHNLLSNQQLLKFRHLIISQVDTSFNKLYELCKNSEDNQGNYTTTTTAAGMVRI | amino acid | |
| DDWLAELSFNVIGRIVCGFQSGPKTGAPSRVEQFKEAINEASYFMSTSPVSDNVP | sequence | |
| MLGWIDQLTGLTRNMKHCGKKLDLVVESIINDHRQKRRFSRTKGGDEKDDEQ | >QCOU- | |
| DDFIDICLSIMEQPQLPGNNNPSQIPIKSIVLDMIGGGTDTTKLTTIWTLSLLLNNP | 2000833 | |
| HVLDKAKQEVDAHFRTKRRSTNDAAAAVVDFDDIRNLVYIQALYPASPVVERL | | |
| SGEDCVVGGFHVPAGTRLWANVWKMQRDPKVWDDPLVFRPDRFLSDEQKMV | | |
| DVRGQNYELLPFGAGRRVCPGVSFSLDLMQLVLTRLILEFEMKSPSGKVDMTA | | |
| TPGLMSYKVIPLDILLTHRRIKPCVQSAASERDMESSGVPVITLGSGKVMPVLG | | |
| MGTFEKVGKGSERERLAILKAIEVGYRYFDTAAAYETEEVLGEAIAEALQLGLV | | |
| KSRDELFISSMLWCTDAHADRVLLALQNSLRNLKLEYVDLYMLPFPASLKPGKI | | |
| TMDIPEEDICRMDYRSVWAAMEE | | |
| MELQYFSYFQPTSSVVALLLALVSILFSVVVLRKTFSNNYSSPASSTETAVLCHQ | P. bracteatum | SEQ. ID |
| RQQSCALPISGLLHVFMNKNGLIHVTLGNMADKYGPIFSFPTGSHRTLVVSSWE | plant source; | NO. 7 |
| MVKECFTGNNDTAFSNRPIPLAFQTIFYACGGIDSYGLSSVPYGKYWRELRKVC | full-length | |
| VHNLLSNQQLLKFRHLIISQVDTSFNKLYELCKNSEDNQGMVRMDDWLAQLSF | amino acid | |
| NVIGRIVCGFQSDPKTGAPSRVEQFKEVINEASYFMSTSPVSDNVPMLGWIDQL | sequence | |
| TGLTRNMKHCGKKLDLVVESIIKDHRQKRRFSRTKGGDEKDDEQDDFIDICLSI | >SSDU- | |
| MEQPQLPGNNSPPQIPIKSIVLDMIGGGTDTTKLTTIWTLSLLLNNPHVLDKAKQ | 2015634 | |
| EVDAHFRKKRRSTDDAAAAVVDFDDIRNLVYIQAIIKESMRLYPASPVVERLSG | (also SSDU- | |
| EDCVVGGFHVPAGTRLWANVWKMQRDPKVWDDPLVFRPERFLSDEQKMVDV | 2015636, | |
| RGQNYELLPFGAGRRICPGVSFSLDLMQLVLTRLILEFEMKSPSGKVDMTATPG | ZSNV- | |
| LMSYKVVPLDILLTHRRIKSCVQLASSERDMESSGVPVITLSSGKVMPVLGMGT | 2027701 | |
| FEKVGKGSERERLAILKAIEVGYRYFDTAAAYETEEVLGEAIAEALQLGLIESRD | RRID- | |
| ELFISSMLWCTDAHPDRVLLALQNSLRNLKLEYLDLYMLPFPASLKPGKITMDI | 2004435) | |
| PEEDICRMDYRSVWSAMEECQNLGFTKSIGVSNFSSKKLQELMATANIPPAVNQ | | |
| VEMSPAFQQKKLREYCNANNILVSAVSILGSNGTPWGSNAVLGSEVLKQIAMA | | |
| KGKSVAQVSMRWVYEQGASLVVKSFSEERLRENLNIFDWELTKEDNEKIGEIP | | |
| QCRILTAYFLVSPNGPFKSQEELWDDKA* | | |
| MELQYFSYFQPTSSVVALLLALVSILFSVVVLRKTFSNNYSSPASSTETAVLCHQ | P. bracteatum | SEQ. ID |
| RQQSCALPISGLLHVFMNKNGLIHVTLGNMADKYGPIFSFPTGSHRTLVVSSWE | plant source; | NO. 8 |
| MVKECFTGNNDTAFSNRPIPLAFQTIFYACGGIDSYGLSSVPYGKYWRELRKVC | full-length | |
| VHNLLSNQQLLKFRHLIISQVDTSFNKLYELCKNSEDNQGMVRMDDWLAQLSF | amino acid | |
| NVIGRIVCGFQSDPKTGAPSRVEQFKEVINEASYFMSTSPVSDNVPMLGWIDQL | sequence | |
| TGLTRNMKHCGKKLDLVVESIIKDHRQKRRFSRTKGGDEKDDEQDDFIDICLSI | >TMWO- | |
| MEQPQLPGNNSPPQIPIKSIVLDMIGGGTDTTKLTTIWTLSLLLNNPHVLDKAKQ | 2027322 | |
| EVDAHFRKKRRSTDDAAAAVVDFDDIRNLVYIQAIIKESMRLYPASPVVERLSG | (also RRID- | |
| EDCVVGGFHVPAGTRLWANVWKMQRDPKVWDDPLVFRPERFLSDEQKMVDV | 2004435) | |
| RGQNYELLPFGAGRRICPGVSFSLDLMQLVLTRLILEFEMKSPSGKVDMTATPG | | |
| LMSYKVVPLDILLTHRRIKSCVQLASSERDMESSGVPVITLSSGKVMPVLGMGT | | |
| FEKVGKGSERERLAILKAIEVGYRYFDTAAAYETEEVLGEAIAEALQLGLIESRD | | |
| ELFISSMLWCTDAHPDRVLLALQNSLRNLKLEYLDLYMLPFPASLKPGKITMDI | | |
| PEEDICRMDYRSVWSAMEECQNLGFTKSIGVSNFSCKKLQELMATANIPPAVN | | |
| QVEMSPAFQQKKLREYCNANNILVSAVSILGSNGTPWGSNAVLGSEVLKQIAM | | |
| AKGKSVAQVSMRWVYEQGASLVVKSFSEERLRENLNIFDWELTKEDNEKIGEI | | |
| PQCRILTAYFLVSPNGPFKSQEELWDDKA* | | |
| SSPASSTETAVLCHQRQQSCALPISGLLHIFMNKNGLIHVTLGNMADKYGPIFSF | P. bracteatum | SEQ. ID |
| PTGSHRILVVSSWEMVKECFTGNNDTAFSNRPIPLAFKTIFYACRGIDSYGLSSV | plant source; | NO. 9 |
| PYGKYWRELRKVCVHNLLSNQQLLKFRHLIISQVDTSFNKLYELCKNSEDNQG | partial-length | |
| MVRMDDWLAQLSFSVIGRIVCGFQSDPKTGAPSRVEQFKEAINEASYFMSTSPV | amino acid | |
| SDNVPMLGWIDQLTGLTRNMTHCGKKLDLVVESIINDHRQKRRFSRTKGGDEK | sequence | |
| DDEQDDFIDICLSIMEQPQLPGNNNPPKIPIKSIVLDMIGAGTDTTKLTIIWTLSLL | >pbr.PBRST | |
| LNNPNVLAKAKQEVDAHFETKKRSTNEASVVVDFDDIGNLVYIQAIIKESMRLY | 1PF_89405 | |
| PVSPVVERLSSEDCVVGGFHVPAGTRLWANVWKMQRDPKVWDDPLVFRPERF | | |
| LSDEQKMVDVRGQNYELLPFGAGRRICPGVSFSLDLMQLVLTRLILEFEMKSPS | | |
| GKVDMTATPGLMSYKVVPLDILLTHRRIKSCVQLASSERDMESSGVPVITLRSG | | |
| KVMPVLGMGTFEKAGKGSERERLAILKAIEVGYRYFDTAAAYETEEVLGEAIA | | |
| EALQLGLIKSRDELFISSMLWCTDAHPDRVLLALQNSLRNLKLEYVDLYMLPFP | | |
| ASLKPGKITMDIPEEDICPMDYRSVWSAMEECQNLGLTKSIGVSNFSCKKLEEL | | |
| MATANIPPAVNQVEMSPAFQQKKLREYCNANNILVSAVSILGSNGTPWGSNAV | | |
| LGSEVLKKIAMAKGKSVAQVSMRWVYEQGASLVVKSFSEERLRENLNIFDWQ | | |
| LTKEDNEKIGEIPQCRILSAYFLVSPKGPFKSQEELWDDKA* | | |
| SSPASSTETAVLCHQRQQSCALPISGLLHIFMNKNGLIHVTLGNMADKYGPIFSF | P. bracteatum | SEQ. ID |
| PTGSHRILVVSSWEMVKECFTGNNDTFFSNRPIPLAFKIIFYAGGVDSYGLALVP | plant source; | NO. 10 |
| YGKYWRELRKICVHNLLSNQQLLKFRHLIISQVDTSFNKLYELCKNSEDNQGM | partial-length | |
| VRMDDWLAQLSFSVIGRIVCGFQSDPKTGAPSRVEQFKEAINEASYFMSTSPVS | amino acid | |
| DNVPMLGWIDQLTGLTRNMTHCGKKLDLVVESIINDHRQKRRFSRTKGGDEKD | sequence | |
| DEQDDFIDICLSIMEQPQLPGNNNPPKIPIKSIVLDMIGGGTDTTKLTTIWTLSLLL | >pbr.PBRST | |
| NNPHVLDKAKQEVDAHFLTKRRSTNDAAVVDFDDIRNLVYIQAIIKESMRLYP | 1PF_4328 | |
| ASPVVERLSGEDCVVGGFHVPAGTRLWVNVWKMQRDPNVWADPMVFRPERF | | |
| LSHGQKKMVDVRGKNYELLPFGAGRRICPGISFSLDLMQLVLTRLILEFEMKSP | | |
| SGKVDMTATPGLMSYKVVPLDILLTHRRIKSCVQLASSERDMESSGVPVITLRS | | |
| GKVMPVLGMGTFEKAGKGSERERLAILKAIEVGYRYFDTAAAYETEEVLGEAI | | |
| AEALQLGLIKSRDELFISSMLWCTDAHPDRVLLALQNSLRNLKLEYVDLYMLPF | | |
| PASLKPGKITMDIPEEDICPMDYRSVWSAMEECQNLGLTKSIGVSNFSCKKLEEL | | |
| MATANIPPAVNQVEMSPAFQQKKLREYCNANNILVSAVSILGSNGTPWGSNAV | | |
| LGSEVLKKIAMAKGKSVAQVSMRWVYEQGASLVVKSFSEERLRENLNIFDWQ | | |
| LTKEDNEKIGEIPQCRILSAYFLVSPKGPFKSQEELWDDKA* | | |
| SSPASSTETAVLCHQRQQSCALPISGLLHIFMNKNGLIHVTLGNMADKYGPIFSF | P. bracteatum | SEQ. ID |
| PTGSHRILVVSSWEMVKECFTGNNDTFFSNRPIPLAFKIIFYAGGVDSYGLALVP | plant source; | NO. 11 |
| YGKYWRELRKICVHNLLSNQQLLNFRHLIISQVDTSFNKLYDLSNKKKNTTTDS | partial-length | |
| GTVRMDDWLAQLSFNVIGRIVCGFQTHTETSATSSVERFTEAIDEASRFMSIATV | amino acid | |
| SDTFPWLGWIDQLTGLTRKMKHYGKKLDLVVESIIEDHRQNRRISGTKQGDDFI | sequence | |
| DICLSIMEQPQIIPGNNDPPRQIPIKSIVLDMIGGGTDTTKLTTTWTLSLLLNNPHV | >pbr.PBRST | |
| LEKAREEVDAHFGTKRRPTNDDAVMVEFDDIRNLVYIQAIIKESMRLYPASPVV | 1PF_12180 | |
| ERLSGEDCVVGGFHVPAGTRLWVNVWKMQRDPNVWADPMVFRPERFLSDEQ | | |
| KMVDVRGQNYELLPFGAGRRICPGVSFSLDLMQLVLTRLILEFEMKSPSGKVD | | |
| MTATPGLMSYKVVPLDILLTHRRIKSCVQLASSERDMESSGVPVITLRSGKVMP | | |
| VLGMGTFEKAGKGSERERLAILKAIEVGYRYFDTAAAYETEEVLGEAIAEALQL | | |
| GLIKSRDELFISSMLWCTDAHPDRVLLALQNSLRNLKLEYVDLYMLPFPASLKP | | |
| GKITMDIPEEDICPMDYRSVWSAMEECQNLGLTKSIGVSNFSCKKLEELMATAN | | |
| IPPAVNQVEMSPAFQQKKLREYCNANNILVSAVSILGSNGTPWGSNAVLGSEVL | | |
| KKIAMAKGKSVAQVSMRWVYEQGASLVVKSFSEERLRENLNIFDWQLTKEDN | | |
| EKIGEIPQCRILSAYFLVSPKGPFKSQEELWDDKA* | | |
| VALRKKILKNYYSSSSSTATAVSHQWPKASRALPLIDLLHVFFNKTDLMHVTLG | P. bracteatum | SEQ. ID |
| NMADKFGPIFSFPTGSHRTLVVSSWEKAKECFTGNNDIVFSGRPLPLAFKLIFYA | plant source; | NO. 12 |
| GGIDSYGISQVPYGKKWRELRNICVHNILSNQQLLKFRHLMISQVDNSFNKLYE | partial-length | |
| VCNSNKDEGDSATSTTAAGIVRMDDWLGKLAFDVIARIVCGFQSQTETSTTSSM | amino acid | |
| ERFTEAMDEASRFMSVTAVSDTVPWLGWIDQLTGLKRNMKHCGKKLNLVVKS | sequence | |
| IIEDHRQKRRLSSTKKGDENIIDEDEQDDFIDICLSIMEQPQLPGNNNPPKIPIKSIV | >pbr.PBRST | |
| LDMIGGGTDTTKLTTIWTLSLLLNNPHVLDKAKQEVDAHFLTKRRSTNDAAVV | 1PF_4329 | |
| DFDDIRNLVYIQAIIKESMRLYPASPVVERLSGEDCVVGGFHVPAGTRLWVNV | | |
| WKMQRDPNVWADPMVFRPERFLSDEQKMVDVRGQNYELLPFGAGRRICPGVS | | |
| FSLDLMQLVLTRLILEFEMKSPSGKVDMTATPGLMSYKVVPLDILLTHRRIKSC | | |
| VQLASSERDMESSGVPVITLRSGKVMPVLGMGTFEKAGKGSERERLAILKAIEV | | |
| GYRYFDTAAAYETEEVLGEAIAEALQLGLIKSRDELFISSMLWCTDAHPDRVLL | | |
| ALQNSLRNLKLEYVDLYMLPFPASLKPGKITMDIPEEDICPMDYRSVWSAMEEC | | |
| QNLGLTKSIGVSNFSCKKLEELMATANIPPAVNQVEMSPAFQQKKLREYCNAN | | |
| NILVSAVSILGSNGTPWGSNAVLGSEVLKKIAMAKGKSVAQVSMRWVYEQGA | | |
| SLVVKSFSEERLRENLNIFDWQLTKEDNEKIGEIPQCRILSAYFLVSPKGPFKSQE | | |
| ELWDDKA* | | |
| MELQYFSYFQPTSSVVALLLALVSILFSVVVLRKTFSNNYSSPASSTETAVLCHQ | P. bracteatum | SEQ. ID |
| RQQSCALPISGLLHVFMNKNGLIHVTLGNMADKYGPIFSFPTGSHRTLVVSSWE | plant source; | NO. 13 |
| MVKECFTGNNDTAFSNRPIPLAFQTIFYACGGIDSYGLSSVPYGKYWRELRKVC | partial-length | |
| VHNLLSNQQLLKFRHLIISQVDTSFNKLYELCKNSEDNQGMVRMDDWLAQLSF | amino acid | |
| NVIGRIVCGFQSDPKTGAPSRVEQFKEVINEASYFMSTSPVSDNVPMLGWIDQL | sequence | |
| TGLTRNMKHCGKKLDLVVESIIKDHRQKRRFSRTKGGDEKDDEQDDFIDICLSI | >SSDU- | |
| MEQPQLPGNNSPPQIPIKSIVLDMIGGGTDTTKLTTIWTLSLLLNNPHVLDKAKQ | 2015635 | |
| EVDAHFRKKRRSTDDAAAAVVDFDDIRNLVYIQAIIKESMRLYPASPVVERLSG | | |
| EDCVVGGFHVPAGTRLWANVWKMQRDPKVWDDPLVFRPERFLSDEQKMVDV | | |
| RGQNYELLPFGAGRRICPGVSFSLDLMQLVLTRLILEFEMKSPSGKVDMTATPG | | |
| LMSYKVVPLDILLTHRRIKSCVQLASSERDMESSGVPVITLSSGKVMPVLGMGT | | |
| FEKVGKGSERERLAILKAIEVGYRYFDTAAAYETEEVLGEAIAEALQLGLIESRD | | |
| ELFISSMLWCTDAHPDRVLLALQNSLRNLKLEYLDLYMLPFPASLKPGKITMDI | | |
| PEEDICRMDYRSVWSAMEECQNLGFTKSIGVSNFSSKKLQELMATANIPPAVNQ | | |
| VEMSPAFQQKKLREYCNANNILVSAVSILGSNGTPWGSNAVLGSEVLKQIAMA | | |
| KGKSVAQVSMRWVXKFSAYAIVWSLFFGHRICITLYSFLIRNVAYICITY* | | |
| MELQYFSYFQPTSSVVALLLALVSILFSVVVLRKTFSNNYSSPASSTETAVLCHQ | P. bracteatum | SEQ. ID |
| RQQSCALPISGLLHVFMNKNGLIHVTLGNMADKYGPIFSFPTGSHRTLVVSSWE | plant source; | NO. 14 |
| MVKECFTGNNDTAFSNRPIPLAFQTIFYACGGIDSYGLSSVPYGKYWRELRKVC | partial-length | |
| VHNLLSNQQLLKFRHLIISQVDTSFNKLYELCKNSEDNQGMVRMDDWLAQLSF | amino acid | |
| NVIGRIVCGFQSDPKTGAPSRVEQFKEVINEASYFMSTSPVSDNVPMLGWIDQL | sequence | |
| TGLTRNMKHCGKKLDLVVESIIKDHRQKRRFSRTKGGDEKDDEQDDFIDICLSI | >SSDU- | |
| MEQPQLPGNNSPPQIPIKSIVLDMIGGGTDTTKLTTIWTLSLLLNNPHVLDKAKQ | 2015637 | |
| EVDAHFRKKRRSTDDAAAAVVDFDDIRNLVYIQAIIKESMRLYPASPVVERLSG | | |
| EDCVVGGFHVPAGTRLWANVWKMQRDPKVWDDPLVFRPERFLSDEQKMVDV | | |
| RGQNYELLPFGAGRRICPGVSFSLDLMQLVLTRLILEFEMKSPSGKVDMTATPG | | |
| LMSYKVVPLDILLTHRRIKSCVQLASSERDMESSGVPVITLSSGKVMPVLGMGT | | |
| FEKVGKGSERERLAILKAIEVGYRYFDTAAAYETEEVLGEAIAEALQLGLIESRD | | |
| ELFISSMLWCTDAHPDRVLLALQNSLRQVFLMQIRLIYICTYQQVHLNIYFQINE | | |
| FVLCDMYRNLKLEY | | |
| LNNYSSSPASSTKTAVLSHQRQQSCALPISGLLHIFMNKNGLIHVTLGNMADKY | C. majus | SEQ. ID |
| GPIFSFPTGSHRTLVVSSWEMVKECFTGNNDTAFSNRPIPLAFKTIFYACGGIDSY | plant source; | NO. 15 |
| GLSSVPYGKYWRELRKVCVHNLLSNQQLLKFRHLIISQVDTSFNKLYELCKNSE | partial-length | |
| DNQGNYPTTTTAAGMVRIDDWLAELSFNVIGRIVCGFQSGPKTGAPSRVEQFKE | amino acid | |
| AINEASYFMSTSPVSDNVPMLGWIDQLTGLTRNMKHCGKKLDLVVESIINDHR | sequence | |
| QKRRFSRTKGGDEKDDEQDDFIDICLSIMEQPQLPGNNNPSQIPIKSIVLDMIGGG | >chm.CMAS | |
| TDTTKLTTIWTLSLLLNNPHVLDKAKQEVDAHFRTKRRSTNDAAAAVVDFDDI | T2PF_14984 | |
| RNLVYIQAIIKESMRLYPASPVVERLSGEDCVVGGFHVPAGTRLWANVWKMQ | | |
| RDPKVWDDPLVFRPDRFLSDEQKMVDVRGQNYELLPFGAGRRVCPGVSFSLDL | | |
| MQLVLTRLILEFEMKSPSGKVDMTATPGLMSYKVIPLDILLTHRRIKPCVQSAAS | | |
| ERDMESSGVPVITLGSGKVMPVLGMGTFEKVGKGSERERLAFLKAIEVGYRYF | | |
| DTAAAYETEEFLGEAIAEALQLGLIKSRDELFITSKLWPCDAHPDLVVPALQNSL | | |
| RNLKLEYVDLYMLPFPASLKPGKITMDIPEEDICRMDYRSVWAAMEECQNLGF | | |
| TKSIGVSNFSCKKLQELMATANIPPAVNQVEMSPAFQQKKLREYCNANNILVSA | | |
| ISVLGSNGTPWGSNAVLGSEVLKKIAMAKGKSVAQVSMRWVYEQGASLVVKS | | |
| FSEERLRENLNIFDWELTKEDHEKIGEIPQCRILSAYFLVSPNGPFKSQEELWDDE | | |
| A* | | |
| MELQYFSYFQPTSSVVALLLALVSILFSVVVLRKTFSNNYSSPASSTETAVLCHQ | P. bracteatum | SEQ. ID |
| RQQSCALPISGLLHVFMNKNGLIHVTLGNMADKYGPIFSFPTGSHRTLVVSSWE | DRS-DRR | NO. 16 |
| MVKECFTGNNDTAFSNRPIPLAFQTIFYACGGIDSYGLSSVPYGKYWRELRKVC | | |
| VHNLLSNQQLLKFRHLIISQVDTSFNKLYELCKNSEDNQGMVRMDDWLAQLSF | | |
| NVIGRIVCGFQSDPKTGAPSRVEQFKEVINEASYFMSTSPVSDNVPMLGWIDQL | | |
| TGLTRNMKHCGKKLDLVVESIIKDHRQKRRFSRTKGGDEKDDEQDDFIDICLSI | | |
| MEQPQLPGNNSPPQIPIKSIVLDMIGGGTDTTKLTTIWTLSLLLNNPHVLDKAKQ | | |
| EVDAHFRKKRRSTDDAAAAVVDFDDIRNLVYIQAIIKESMRLYPASPVVERLSG | | |
| EDCVVGGFHVPAGTRLWANVWKMQRDPKVWDDPLVFRPERFLSDEQKMVDV | | |
| RGQNYELLPFGAGRRICPGVSFSLDLMQLVLTRLILEFEMKSPSGKVDMTATPG | | |
| LMSYKVVPLDILLTHRRIKSCVQLASSERDMESSGVPVITLSSGKVMPVLGMGT | | |
| FEKVGKGSERERLAILKAIEVGYRYFDTAAAYETEEVLGEAIAEALQLGLIESRD | | |
| ELFISSMLWCTDAHPDRVLLALQNSLRNLKLEYLDLYMLPFPASLKPGKITMDI | | |
| PEEDICRMDYRSVWSAMEECQNLGFTKSIGVSNFSCKKLQELMATANIPPAVN | | |
| QVEMSPAFQQKKLREYCNANNILVSAVSILGSNGTPWGSNAVLGSEVLKQIAM | | |
| AKGKSVAQVSMRWVYEQGASLVVKSFSEERLRENLNIFDWELTKEDNEKIGEI | | |
| PQCRILTAYFLVSPNGPFKSQEELWDDKA* | | |
| MELQYFSYFQPTSSVVALLLALVSILFSVVVLRKTFSNNYSSPASSTETAVLCHQ | P. bracteatum | SEQ. ID |
| RQQSCALPISGLLHVFMNKNGLIHVTLGNMADKYGPIFSFPTGSHRTLVVSSWE | DRS | NO. 17 |
| MVKECFTGNNDTAFSNRPIPLAFQTIFYACGGIDSYGLSSVPYGKYWRELRKVC | | |
| VHNLLSNQQLLKFRHLIISQVDTSFNKLYELCKNSEDNQGMVRMDDWLAQLSF | | |
| NVIGRIVCGFQSDPKTGAPSRVEQFKEVINEASYFMSTSPVSDNVPMLGWIDQL | | |
| TGLTRNMKHCGKKLDLVVESIIKDHRQKRRFSRTKGGDEKDDEQDDFIDICLSI | | |
| MEQPQLPGNNSPPQIPIKSIVLDMIGGGTDTTKLTTIWTLSLLLNNPHVLDKAKQ | | |
| EVDAHFRKKRRSTDDAAAAVVDFDDIRNLVYIQAIIKESMRLYPASPVVERLSG | | |
| EDCVVGGFHVPAGTRLWANVWKMQRDPKVWDDPLVFRPERFLSDEQKMVDV | | |
| RGQNYELLPFGAGRRICPGVSFSLDLMQLVLTRLILEFEMKSPSGKVDMTATPG | | |
| LMSYKVVPLDILLTHRRIKSCVQLASSERD | | |
| MESSGVPVITLSSGKVMPVLGMGTFEKVGKGSERERLAILKAIEVGYRYFDTAA | P. bracteatum | SEQ. ID |
| AYETEEVLGEAIAEALQLGLIESRDELFISSMLWCTDAHPDRVLLALQNSLRNL | DRR | NO. 18 |
| KLEYLDLYMLPFPASLKPGKITMDIPEEDICRMDYRSVWSAMEECQNLGFTKSI | | |
| GVSNFSCKKLQELMATANIPPAVNQVEMSPAFQQKKLREYCNANNILVSAVSIL | | |
| GSNGTPWGSNAVLGSEVLKQIAMAKGKSVAQVSMRWVYEQGASLVVKSFSEE | | |
| RLRENLNIFDWELTKEDNEKIGEIPQCRILTAYFLVSPNGPFKSQEELWDDKA* | | |

### Morphinan Alkaloid Generating Modifications

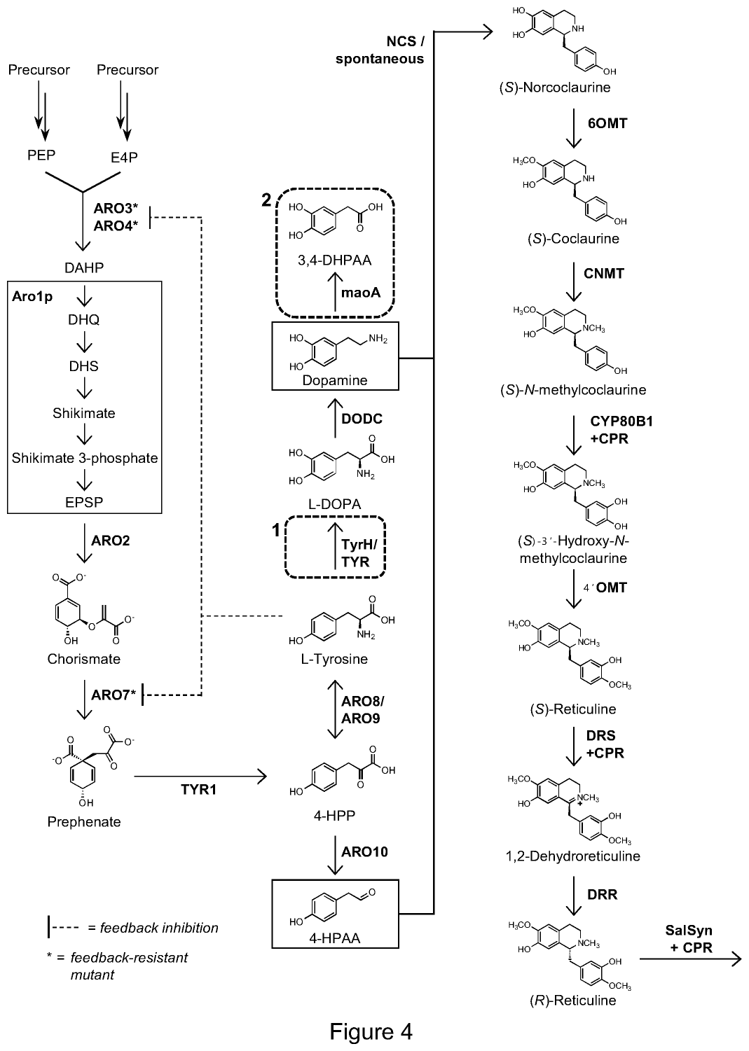

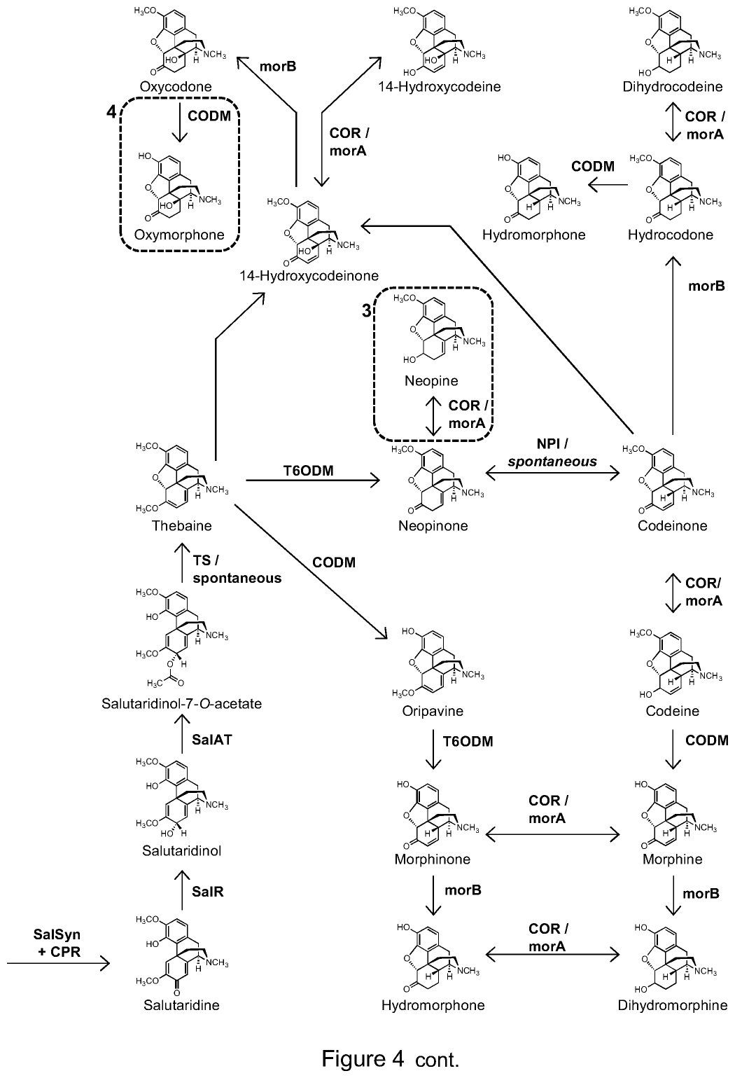

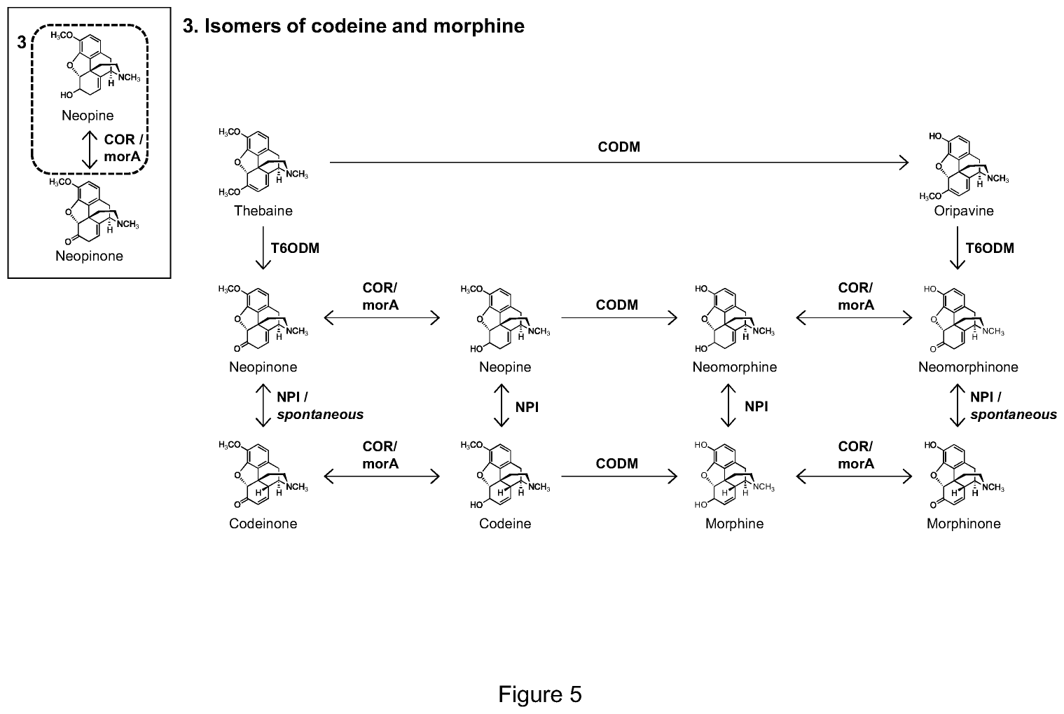

Some methods, processes, and systems provided herein describe the conversion of promorphinan alkaloids to morphinan alkaloids. Some of the methods, processes, and systems describe the conversion of a tetracyclic scaffold to a pentacyclic scaffold (FIG. 4). Some of the methods, processes, and systems may comprise an engineered host cell. In some examples, the production of pentacyclic thebaine, or a morphinan alkaloid, from a tetracyclic precursor, or a promorphinan alkaloid is described. In some examples, the conversion of promorphinan alkaloids to thebaine are key steps in the conversion of a substrate to a diverse range of benzylisoquinoline alkaloids.

In some examples, the tetracyclic precursor may be salutaridine, salutaridinol, or salutaridinol-7-O-acetate. The tetracyclic precursor may be converted to pentacyclic thebaine by closure of an oxide bridge between C-4 and C-5. In some examples, the tetracyclic precursor salutaridine may be prepared for ring closure by stepwise hydroxylation and O-acetylation at C-7. Ring closure may be activated by elimination of an acetate leaving group. In some examples, the allylic elimination and oxide ring closure that generates thebaine occurs spontaneously. In other examples, the ring closure reaction that generates pentacyclic thebaine is promoted by factors such as pH or solvent. In other examples, the thebaine-generating ring closure reaction is promoted by contact with a protein or enzyme. These conversion steps are provided in FIG. 4 and represented generally in Scheme 2. R₁, R₂, and R₃ may be H or CH₃. R₄ may be CH₃, CH₃CH₂, CH₃CH₂CH₂, or other appropriate alkyl group. In some cases, R₁, R₂, R₃, and R₄ may be CH₃ as provided in FIG. 4.

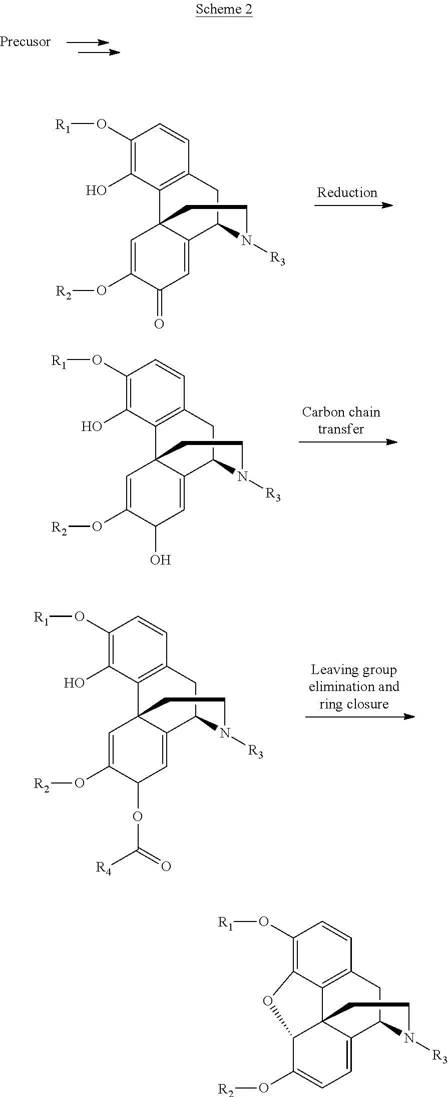

In some examples, the first enzyme that prepares the tetracyclic precursor is salutaridine reductase (SalR). In some cases, SalR hydroxylates the substrate salutaridine at the C-7 position (see Formula III). The product of this reaction may be one or more salutaridinol epimers. In some examples, the product is (7S)-salutaridinol. In some examples, the salutaridine reductase may catalyze the reduction reaction within a host cell, such as an engineered host cell, as described herein. In some examples, the second enzyme that prepares the tetracyclic precursor is salutaridinol 7-O-acetyltransferase (SalAT). In some cases, SalAT transfers the acetyl from acetyl-CoA to the 7-OH of salutaridinol (see Formula IV). In other cases, SalAT may utilize a novel cofactor such as n-propionyl-CoA and transfer the propionyl to the 7-OH of salutaridinol. In some examples, the product of SalAT is (7S)-salutaridinol-7-O-acetate. In some examples, the salutaridinol 7-O-acetyltransferase may catalyze the acetyl transfer reaction within a host cell, such as an engineered host cell, as described herein.

In some examples, the tetracyclic precursor of thebaine is (7S)-salutaridinol-7-O-acetate. In some examples (7S)-salutaridinol-7-O-acetate is unstable and spontaneously eliminates the acetate at C-7 and closes the oxide bridge between C-4 and C-5 to form thebaine (see Formula V). In some examples, the rate of elimination of the acetate leaving group is promoted by pH. In some examples, the allylic elimination and oxide bridge closure is catalyzed by an enzyme with thebaine synthase activity, or a thebaine synthase. In some examples, this enzyme is a Bet v 1-fold protein. In some examples, this enzyme is an engineered thebaine synthase, an engineered SalAT, a dirigent (DIR) protein, or a chalcone isomerase (CHI). In some examples, the enzyme encoding thebaine synthase activity may catalyze the ring closure reaction within a host cell, such as an engineered host cell, as described herein.

In some examples, the salutaridine reductase enzyme may be SalR or a SalR-like enzyme from plants in the Ranunculales order that biosynthesize thebaine, for example Papaver somniferum. In other examples, the enzyme with salutaridine reductase activity may be from mammals or any other vertebrate or invertebrate that biosynthesizes endogenous morphine.

In some examples, the salutaridinol 7-O-acetyltransferase enzyme may be SalAT or a SalAT-like enzyme from plants in the Ranunculales order that biosynthesize thebaine, for example P. somniferum. In other examples, the enzyme with salutaridinol 7-O-acetyltransferase activity may be from mammals or any other vertebrate or invertebrate that biosynthesizes endogenous morphine.

In some examples, the thebaine synthase (TS) enzyme may be a Bet v 1 fold protein from plants in the Ranunculales order that biosynthesize thebaine, for example P. somniferum. In some examples, the Bet v 1 protein includes the following domains in order from the N-terminus to C-terminus: a β-strand, one or two α-helices, six β-strands, and one or two α-helices. The protein is organized such that it has a Bet v 1 fold and an active site that accepts large, bulky, hydrophobic molecules, such as morphinan alkaloids. This protein may be any plant Bet v 1 protein, pathogenesis-related 10 protein (PR-10), a major latex protein (MLP), fruit or pollen allergen, plant hormone binding protein (e.g., binding to cytokinin or brassinosteroids), plant polyketide cyclase-like protein, or norcoclaurine synthase (NCS)-related protein that has a Bet v 1 fold. Other non-plant examples of the Bet v 1 fold protein are polyketide cyclases, activator of Hsp90 ATPase homolog 1 (AHA1) proteins, SMU440-like proteins (e.g., from Streptococcus mutans), PA1206-related proteins (e.g., from Pseudomonas aeruginosa), CalC calicheamicin resistance protein (e.g., from Micromonospora echinospora), and the CoxG protein from carbon monoxide metabolizing Oligotropha carboxidovorans. Further examples from Bet v 1-related families include START lipid transfer proteins, phosphatidylinositol transfer proteins, and ring hydroxylases.

In some examples, the thebaine synthase enzyme may be a dirigent protein from plants in the Ranunculales order that biosynthesize thebaine, for example P. somniferum. In other examples, the enzyme may be any dirigent protein from plants.

In some examples, the thebaine synthase enzyme may be a chalcone isomerase protein from plants in the Ranunculales order that biosynthesize thebaine, for example P. somniferum. In other examples, the enzyme may be any chalcone isomerase protein from plants.

In some examples, the thebaine synthase enzyme may be a SalAT-like enzyme from plants in the Ranunculales order that biosynthesize thebaine, for example P. somniferum. In other examples, the enzyme may be any SalAT-like protein from plants.

In some examples, the enzyme with thebaine synthase activity may be from mammals or any other vertebrate or invertebrate that biosynthesizes endogenous morphine.

In some examples, combinations of the above enzymes together with additional accessory proteins may function to convert various tetracyclic precursors into thebaine. In some examples, these enzymes catalyze the reactions within a host cell, such as an engineered host cell, as described herein.

Examples of amino acid sequences for thebaine synthase activity are set forth in Table 2. An amino acid sequence for a thebaine synthase that is utilized in a tetracyclic precursor to thebaine may be 50% or more identical to a given amino acid sequence as listed in Table 2. For example, an amino acid sequence for such a thebaine synthase may comprise an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the nucleic acid sequence encoding the amino acid sequence is altered such as to optimize codon usage for the host organism, for example.

An engineered host cell may be provided that produces a salutaridine reductase, salutaridinol 7-O-acetyltransferase, and thebaine synthase that converts a tetracyclic precursor into thebaine, wherein the thebaine synthase comprises an amino acid sequence selected from the group consisting of SEQ ID NOs: 19, 20, 21, 22, 23, 24, 25, and 26 as listed in Table 2. In some cases, the thebaine synthase may form a fusion protein with other enzymes. The enzymes that are produced within the engineered host cell may be recovered and purified so as to form a biocatalyst. These one or more enzymes may also be used to catalyze the conversion of a tetracyclic promorphinan precursor to thebaine.

In other examples, the thebaine synthase comprises an amino acid sequence selected from the group consisting of SEQ ID NOs: 27, 28, 29, 30, 31, 32, 33, and 34 as listed in Table 2.

In additional cases, the one or more enzymes that are recovered from the engineered host cell may be used in a process for converting a tetracyclic promorphinan precursor to a thebaine. The process may include contacting the tetracyclic promorphinan precursor with the recovered enzymes in an amount sufficient to convert said tetracyclic promorphinan precursor to thebaine. In some examples, the tetracyclic promorphinan precursor may be contacted with a sufficient amount of the one or more enzymes such that at least 5% of said tetracyclic promorphinan precursor is converted to thebaine. In further examples, the tetracyclic promorphinan precursor may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said tetracyclic promorphinan precursor is converted to thebaine.

In some examples, process conditions are implemented to support the formation of thebaine in engineered host cells. In some cases, engineered host cells are grown at pH 3.3, and once high cell density is reached the pH is adjusted to pH 8.0 to support continued production of thebaine at higher pH. In some cases, the engineered host cells produce additional enzymes to convert sugar and other simple precursors, such as tyrosine, to thebaine. In some cases, the SalAT enzyme has been engineered to exhibit higher activity at pH 8.0 and is expressed from a late stage promoter.

In some examples, one or more of the enzymes converting a tetracyclic promorphinan precursor to a thebaine are localized to cellular compartments. In some examples, SalR, SalAT, and thebaine synthase (TS) may be modified such that they encode targeting sequences that localize them to the endoplasmic reticulum membrane of the engineered host cell. In particular, in certain instances, the host cell may be engineered to increase production of salutaridinol or thebaine or products for which thebaine is a precursor from reticuline or its precursors by localizing TS and/or SalR and/or SalAT to organelles in the yeast cell. TS and/or SalR and/or SalAT may be localized to the yeast endoplasmic reticulum in order to decrease the spatial distance between TS and/or SalR and/or SalAT and CYP2D2 or CYP2D6 or SalSyn or an engineered cytochrome P450 enzyme that catalyzes the conversion of reticuline to salutaridine. By increased production is meant both the production of some amount of the compound of interest where the control has no production of the compound of interest, as well as an increase of 10% or more, such as 50% or more, including 2-fold or more, e.g., 5-fold or more, such as 10-fold or more in situations where the control has some production of the compound of interest.

In other examples, SalAT and TS may be co-localized into a single protein fusion. In some examples, the fusion is created between SalAT and TS by one of several methods, including, direct fusion, co-localization to a yeast organelle, or by enzyme co-localization tools such as leucine zippers, protein scaffolds that utilize adaptor domains, or RNA scaffolds that utilize aptamers. Co-localizing the thebaine synthesis enzyme may facilitate substrate channeling between the active sites of the enzymes and limit the diffusion of unstable intermediates such as salutaridinol-7-O-acetate.

In some examples, an engineered salutaridinol 7-O-acetyltransferase (SalAT) enzyme is used in converting a tetracyclic promorphinan precursor to a thebaine. In some examples, a SalAT enzyme is engineered to combine two functions: (1) the transfer of an acyl group from acetyl-CoA to the 7-OH of salutaridinol, and (2) the subsequent elimination of the acetyl group and closure of an oxide bridge between carbons C4 and C5 to form thebaine.

In some examples, an enzyme with salutaridinol 7-O-acetyltransferase activity is fused to a peptide with a Bet v 1 fold. In some examples, salutaridinol 7-O-acetyltransferase enzyme and the Bet v 1 fold protein may be fused in any order from N-terminus to C-terminus, C-terminus to N-terminus, N-terminus to N-terminus, or C-terminus to C-terminus. In some examples, the two protein sequences may be fused directly or fused through a peptide linker region.

In some examples, an enzyme with salutaridinol 7-O-acetyltransferase activity is fused to a peptide with a Bet v 1 fold by circular permutation. In some cases, the N- and C-termini of SalAT are fused and the Bet v 1 sequence is then inserted randomly within this sequence. In some cases, the resulting fusion protein library is screened for thebaine production. In other cases, a circular permutation SalAT library is first screened for activity in the absence of Bet v 1. In other cases, the N- and C-termini of SalAT are fused and the enzyme is digested and blunt end cloned. In other cases, this library of circularly permuted SalAT is screened for salutaridinol 7-O-acetyltransferase activity. In other cases, active variants from the circularly permuted SalAT library are then used to design protein fusions with a peptide with a Bet v 1 fold.

The one or more enzymes that may be used to convert a tetracyclic promorphinan precursor to a thebaine may contact the tetracyclic promorphinan precursor in vitro. Additionally, or alternatively, the one or more enzymes that may be used to convert a tetracyclic promorphinan precursor to thebaine may contact the tetracyclic promorphinan precursor in vivo. Additionally, the one or more enzymes that may be used to convert a tetracyclic promorphinan precursor to thebaine may be provided to a cell having the tetracyclic promorphinan precursor within, or may be produced within an engineered host cell.

In some examples, the methods provide for engineered host cells that produce an alkaloid product, wherein the conversion of a tetracyclic promorphinan precursor to a thebaine may comprise a key step in the production of an alkaloid product. In some examples, the alkaloid product is a thebaine. In still other embodiments, the alkaloid product is derived from a thebaine, including for example, downstream morphinan alkaloids. In another embodiment, a tetracyclic promorphinan precursor is an intermediate toward the product in of the engineered host cell. In still other embodiments, the alkaloid product is selected from the group consisting of morphinan, nor-opioid, or nal-opioid alkaloids.

In some examples, the substrate of the reduction reaction is a compound of Formula III:

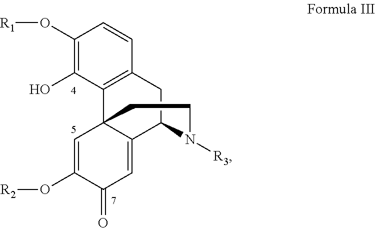

or a salt thereof, wherein:
R₁, R₂, and R₃ are independently selected from hydrogen and methyl.

In some other examples, R₁, R₂, and R₃ are methyl, and the reduction reaction is catalyzed by a salutaridine reductase.

In some examples, the substrate of the carbon chain transfer reaction is a compound of Formula IV:

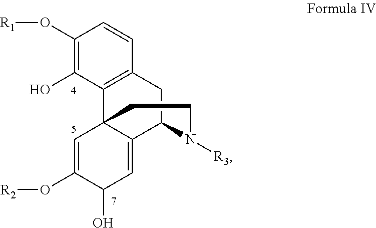

or a salt thereof, wherein:
R₁, R₂, and R₃ are independently selected from hydrogen and methyl.

In some other examples, R₁, R₂, and R₃ are methyl, and the carbon chain transfer reaction is catalyzed by a salutaridinol 7-O-acetyltransferase.

In some examples, the substrate of thebaine synthase is a compound of Formula V:

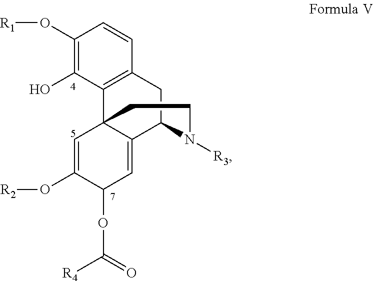

or a salt thereof, wherein:
R₁, R₂, and R₃ are independently selected from hydrogen and methyl; and
R₄ is selected from methyl, ethyl, propyl, and other appropriate alkyl group.

In some other examples, R₁, R₂, R₃, and R₄ are methyl, and the ring closure reaction is catalyzed by a thebaine synthase. In some examples, the thebaine synthase is a Bet v 1 protein. In some examples, the methods provide for engineered host cells that produce alkaloid products from salutaridine. The conversion of salutardine to thebaine may comprise a key step in the production of diverse alkaloid products from a precursor. In some examples, the precursor is L-tyrosine or a sugar (e.g., glucose). The diverse alkaloid products can include, without limitation, morphinan, nor-opioid, or nal-opioid alkaloids. Any suitable carbon source may be used as a precursor toward a pentacyclic morphinan alkaloid. Suitable precursors can include, without limitation, monosaccharides (e.g., glucose, fructose, galactose, xylose), oligosaccharides (e.g., lactose, sucrose, raffinose), polysaccharides (e.g., starch, cellulose), or a combination thereof. In some examples, unpurified mixtures from renewable feedstocks can be used (e.g., cornsteep liquor, sugar beet molasses, barley malt, biomass hydrolysate). In still other embodiments, the carbon precursor can be a one-carbon compound (e.g., methanol, carbon dioxide) or a two-carbon compound (e.g., ethanol). In yet other embodiments, other carbon-containing compounds can be utilized, for example, methylamine, glucosamine, and amino acids (e.g., L-tyrosine). In some examples, a 1-benzylisoquinoline alkaloid may be added directly to an engineered host cell of the disclosure, including, for example, norlaudanosoline, laudanosoline, norreticuline, and reticuline.

In some examples, the benzylisoquinoline alkaloid product, or a derivative thereof, is recovered. In some examples, the benzylisoquinoline alkaloid product is recovered from a cell culture. In some examples, the benzylisoquinoline alkaloid product is a morphinan, nor-opioid, or nal-opioid alkaloid.

TABLE 2
Example amino acid sequences of morphinan alkaloid generating enzymes.

| Sequence | | | SEQ. ID |
|---|---|---|---|
| Name | Description | Sequence | NO. |
| Bet v1 | | | |
| | P. bracteatum | MAPRGVSGLVGKLSTELDVNCDAEKYYNMYKNGEDVQKAVPHLC | SEQ. ID. |
| | | MDVKVISGDATRSGCIKEWNVNIDGKTIRSVEETTHNDETKTLRH | NO. 19 |
| | | RVFEGDMMKDYKKFDTIMEVNPKPDGNGCVVTRSIEYEKVNENS | |
| | | PTPFDYLQFGHQAMEDMNKY | |
| | P. setigerum | MLVGKLSTELEVDCDAEKYYNMYKHGEDVKKALCVDVKVISGDP | SEQ. ID. |
| | | TRSGCIKEWNVNIDGKTIRSVEETTHNDETKTLRHRVFEGDMMK | NO. 20 |
| | | DFKKFDTIMVVNPKPDGNGCVVTRSIEYEKTNENSPTPFDYLQFG | |
| | | HQAIEDMNKYL | |
| | P. setigerum | MLVGKLSTELEVDCDAEKYYNMYKHGEDKRQCVDVKVISGDPTR | SEQ. ID. |
| | | SGCIKEWNVNIDGKTIRSVEETTHNDETKTLRHRVFEGDMMKDF | NO. 21 |
| | | KKFDTIMVVNPKPDGNGCVVTRSIEYEKTNENSPTPFDYLQFGHQ | |
| | | AIEDMNKY | |
| | P. setigerum | MLVGKLSTELEVDCDAEKYYNMYKHGEDVKKAVPHLCVDVKIISG | SEQ. ID. |
| | | DPTSSGCIKEWNVNIDGKTIRSVEETTHDDETKTLRHRVFEGDVM | NO. 22 |
| | | KDFKKFDTIMVVNPKPDGNGCVVTRSIEYEKTNENSPTPFDYLQF | |
| | | GHQAIEDMNKYL | |
| | P. setigerum | MVKIISGDPTSSGCIKEWNVNIDGKTIRSVEETTHDDETKTLRHRV | SEQ. ID. |
| | | FEGDVMKDFKKFDTIMVVNPKPDGNGCVVTRSIEYEKTNENSPTP | NO. 23 |
| | | FDYLQFGHQAIEDMNKYL | |
| | P. | MDSINSSIYFCAYFRELIIKLLMAPPGVSGLVGKLSTELEVNCDAEK | SEQ. ID. |
| | somniferum | YYNMYKHGEDVQKAVPHLCVDVKVISGDPTRSGCIKEWNVNIDG | NO. 24 |
| | | KTIRSVEETTHNDETKTLRHRVFEGDVMKDFKKFDTIMVVNPKP | |
| | | DGNGCVVTRSIEYEKTNDNSPTPFDYLQFGHQAIEDMNKYLRDSE | |
| | P. | MNFFIKDHLYICLVGKLSTELEVDCDAEKYYNMYKHGEDVKKAVP | SEQ. ID. |
| | somniferum | HLCVDVKIISGDPTSSGCIKEWNVNIDGKTIRSVEETTHDDETKTL | NO. 25 |
| | | RHRVFEGDVMKDFKKFDTIMVVNPKPDGNGCVVTRSIEYEKTNE | |
| | | NSPTPFDYLQFGHQAIEDMNKYLRDSESN | |
| | P. | MAPLGVSGLVGKLSTELEVDCDAEKYYNMYKHGEDVKKAVPHLC | SEQ. ID. |
| | somniferum | VDVKIISGDPTSSGCIKEWNVNIDGKTIRSVEETTHDDETKTLRHR | NO. 26 |
| | | VFEGDVMKDFKKFDTIMVVNPKPDGNGCVVTRSIEYEKTNENSP | |
| | | TPFDYLQFGHQAIEDMNKYLRDSESN | |
| SalAT | | | |
| | P. | MMKVCVSSREKIKPSRPTPGHLKTHKLSFLDQVAARIYVPLLLYYA | SEQ. ID. |
| | somniferum | GNKENVDTDTRCNIIKKSLAETLTKFYILAGKIVNDEIERFVNCND | NO. 27 |
| | | DGVDFCVTKVSNCQLFQVIKRPDIFDQVTLFLPFDPCDNEITASGD | |
| | | FLLSVQVNVFEDCRGMVIGLCINHKVADASSITTFVNYWATIARGL | |
| | | VLNVDDRQIQDPCFQVQSIFPQKEKGIGFKISSSSIDGTLVTKKFGF | |
| | | EASKLAELKERCKFAGATEDIRGGYKPNRVEALSTFLWKCFIDIDQ | |
| | | AKTKAAAPARVYLASNAVNIRSRIVPQLPTSSFGNMVAITDAIFTV | |
| | | NSNENNGINDPYYPKLVQKFRDAVKRVDGEYIEALQSTDLLLNNV | |
| | | TKLFKHILNGQTLSISFTSWCRFPFYDTDLLD | |
| | P. | MKVQVISKELIKPSTPTPPRLRNFKLSLLDQLLPPFYVPIIIFYPAND | SEQ. ID. |
| | somniferum | DHESNNNDQCIKANILKKSLSETLTRFYPIAGRIRDKILVECNDEG | NO. 28 |
| | | VHYIEAKVNAVMSDFMSLDVIHQLHPSYITLDDLAEEAQLAVQVT | |
| | | MFDCGGIALSICSSHKIIDGCTSTTFLNSWAATARAPSNPEIVYPTF | |
| | | DAAAIFPAQPSGVQVSTLESDDRLQGENVVTKRFLFSASKITALRA | |
| | | RIAESRSSNILSKYPSRSEAVSALVWKSFMETSRVKVTREHTFSAE | |
| | | ASTKPIVRSIANFVVNLRTRLNPPLPNVSFGNIIMDATAESLIIDNG | |
| | | ENTLGFVETLDGLISQLRLGVTKMDDEYVRKLREDDVEFLKSLDE | |
| | | ASHPSNGEGDGNGERV | |
| | P. setigerum | MNDTMKIEVVSKESIKPSYPTPNNLKIHNLSNLDQLIPAFYMDHIL | SEQ. ID. |
| | | YYPSLDSNDSSLGDDEEDKKMIFSASSRHRCDVVKKSLAETLTRYY | NO. 29 |
| | | PLAGRIKDEKSVECNDEGVDYIEARVVGITVSQVIQLASSDIEVMEP | |
| | | FLPYEPYGGTGSAFRRAGIHSNSKPLLKIQVNVFDCGGMVICLSGS | |
| | | HKVIDATSILNFVNDWAATARGGFDTHDDELKVAVVDKPCYIFSS | |
| | | MFPPTSFGNQEEKDTADQAQLVPDRIEIVTKRFVFKDSSIAKLKKK | |
| | | CIHVNTNNGSDHQVDKQEHNMQQMPSRIEALTSLIWMCFMDVD | |
| | | RRFRVKQIDDAVSPVNTVNEVSLPKQVQYVAGFAINLRTRTIQPLP | |
| | | TNSFGNMTDTAIAEVTLNLTGSDHFNNEKGIRDQSQNYPELVSKI | |
| | | KDSIKLVDNKHIEAMKRNLAISCNNIKMHQMMKESTFDQNTREL | |
| | | LMFSSWCRFPIYEADFGWGKPSWASITKLLYKNCVMFLDTSSGDG | |
| | | IEAWVSLKEEDMVEFERHEELVALAS | |
| | P. | MKVQVISKEIIKPSSPTPPHLRNFKLSLLDQILPPFYVPIVMFYPAG | SEQ. ID. |
| | somniferum | DDYVTNNNIHDQSSKSEFLKKSLSETLTRFYPIAGRIKDNILIDCNN | NO. 30 |
| | | EGVDYIEAKVNGIMSDFMSVDVVHQLHPSHIMLDDVAKEAQLAV | |
| | | QVNLFDCGGIAISISMSHKIVDACTAITFINGWAATARAAPKQEIVC | |
| | | PTFDSAAIFPALPPGVQVSSLESDDSVQGVNVVTKMFAFTAPKIAS | |
| | | LRARIAELRSSSDGLSKYPTRTEALSALVWKSFIRTSRVKAARKYS | |
| | | LSPASTKPVIKSVANYAVNLRTRLNPPLPQVSFGNILMDATAESTT | |
| | | TIDDDDSHEFADTLAGLIGQLRLGVSRINGDYIRKLQEGDLAFLKS | |
| | | LDEASHDSNGEKVQICWISSLCRFPFYEADFGWGKPSWVALNTN | |
| | | AEYKNSLFLMDTKCGTGIEAWVSLEEDDMAIFEEDQDLLQCVKSI | |
| | | N | |
| | P. setigerum | MENMKVEVVLKQTIKPSTQTPLHSKTFNLSFLDQHLGPPIYIPFTL | SEQ. ID. |
| | | YYESGDVNNKNNHCDGYKNNLEEACEHRVSVIKQSLSETLARYYP | NO. 31 |
| | | LAGRMKEDNLAVECNDEGVEYFETRVSDVRLSQVIKRSPNHNSVL | |
| | | RKFLPPCISSCDNSMSIPFDYGFKSKTLLAIQVNIFECGGIVIGMCM | |
| | | AHRLADASTMFTFITDWAATARGAIEDIKGPSFDFSYTLFPQKDVI | |
| | | NNFKPFDPMLTREEDLVTKYFVFPASKIVELKRRNVNNIVCQDTS | |
| | | QQNTSPCTRVEAVTSFMWKRYMDSVRAKNQTQATSVEKYGALY | |
| | | TVNLRSRITPPLPANSFGNIYTFTIALSTPSDENDIDDGLRKDVSSP | |
| | | NDLNLVGKVRDAIKKIDDKYTRKLQSSEDELVNDVKPLTSGEAIFL | |
| | | GFSSWCRFPIYEADFGWGKPTWVSIGTMALRNTVFLMDTKSGDG | |
| | | IEAFVNMAKEDMDNFEVKLLADQ | |
| | P. setigerum | MENMKVEVVLEQTIKPSTQTPLHSKTFNLSFLDQHLGPPIYIPFTL | SEQ. ID. |
| | | YYESGDVNNKNNHCDGYKNNLEEVCEHRVSVIKQSLSETLARYYP | NO. 32 |
| | | LAGRMKEDNLAVECNDEGVEYFETRVSDVRLSQVIKRSPNHNSVL | |
| | | RKFLPPCISSCDNSMSIPFDYGFKSKTLLAIQVNIFECGGIVIGMCM | |
| | | AHRLADASTMFTFITDWAATARGAIEDIKGPSFDFSYTLFPQKDVI | |
| | | NNFKPFDPMLTREEDLVTKYFVFPASKIVELKRRNVNNIVCQDTS | |
| | | QQNTSPCTRVEAVTSFMWKRYMDSVRAKNQTQATSVEKYGALY | |
| | | TVNLRSRITPPLPANSFGNIYTFTIALSTPSDENDIDDGLRKDVSSP | |
| | | NDLNLVGKVRDAIKKIDDKYTRKLQSSEDELVNDVKPLTSGEAIFL | |
| | | GFSSWCRFPIYEADFGWGKPTWVSIGTMALRNTVFLMDTKSGDG | |
| | | IEAFVNMAKEDMDNFEVKLLADQLLHVHPTV | |
| | P. setigerum | MSSTVEVISKQTIKPSTPTPIQRKNHSLSLIDQHFAPIYIPIVLFYPA | SEQ. ID. |
| | | AAVNDTGNVQHGDNTCVLKRSLSETLVHFYPLAGRMKDNIVVDC | NO. 33 |
| | | NDQGVEFTEVKVSGTMCDFLMKPDEQLSGLLPSEAVCMNFVREA | |
| | | QVMIQVNTFDCGSKAISLCVSHKIADASTITTFSRCWAETTIAVSKS | |
| | | TSAVTPIVSSKFHPTFDAASLFPPIKQLISPSGVTPALPELIPSEESK | |
| | | FGKIISKRFLFSATTINSVREKLSALMADKLKYRRLTRVEVVSALIW | |
| | | NSFDKLATTGSVAVMVKHAVNLRKRIDPPLPDVSFGNILEFTKAV | |
| | | VGEAAANTTTQGTVGSSSKLLEELSEFAGQLREPVSKMNKGDHDF | |
| | | DMENTDYEERDLWMSSWCNYGLYDIDFGCGKPVWVTTVATMYP | |
| | | YSDGFFMNDTRCGQGIEVWGNLVEEDMANFQLNLSELLDRI | |
| | P. | MMKVCVSSREKIKPSRPTPGHLKTHKLSFLDQVAARIYVPLLLYYA | SEQ. ID. |
| | somniferum | GNKENVDTDTRCNIIKKSLAETLTKFYILAGKIVNDEIERFVNCND | NO. 34 |
| | | DGVDFCVTKVSNCQLFQVIKRPDIFDQVTLFLPFDPCDNEITASGD | |
| | | FLLSVQVNVFEDCRGMVIGLCINHKVADASSITTFVNYWATIARGL | |
| | | VLNVDDRQIQDPCFQVQSIFPQKEKGIGFKISSSSIDGTLVTKKFGF | |
| | | EASKLAELKERCKFTTEPEDGYKPTRVEALSAFLWKCFIDIDQAKL | |
| | | KGVARTKVYLATNAVNMRSRMVPQLPTSSFGNIISITDAVFSINND | |
| | | DSTGINDPYYPKLVRKFRDAIKKIDRDYIEALRSTDLLLNNMMKLI | |
| | | EHVLSGHTLSIYFSSWCRFPLYETDFGWGKPIWVSTCTIPQKNVIV | |
| | | LMDSNSSADGIEAYVTLAKEDMGELEHHEELLALIS | |

### Morphinan Alkaloid Isomerization Modifications

Some methods, processes, and systems provided herein describe the production of morphinan alkaloid isomers. Some of the methods, processes, and systems describe the conversion of a precursor morphinan alkaloid with a carbon-carbon double bond between carbons C-14 and C-8 into a product morphinan alkaloid with a carbon-carbon double bond between carbons C-8 and C-7 (FIG. 4). Some of the methods, processes, and systems may comprise an engineered host cell. In some examples, the conversion of a precursor morphinan alkaloid with a carbon-carbon double bond between carbons C-14 and C-8 into a product morphinan alkaloid with a carbon-carbon double bond between carbons C-8 and C-7 are significant steps in the conversion of a precursor to a diverse range of benzylisoquinoline alkaloids.

In some examples, the production of precursor morphinan alkaloids with a carbon-carbon double bond between carbons C-14 and C-8 occurs within the engineered host cell comprising a plurality of heterologous enzymes for converting simple starting materials to the precursor morphinan alkaloids. In some examples, the simple starting materials are sugar and/or L-tyrosine.

In some examples, the isomer precursor morphinan alkaloid may be neopinone, neopine, neomorphine, or neomorphinone. The precursor morphinan alkaloid may be converted to the desired isomer by rearrangement of a carbon-carbon double bond between carbons C-14 and C-8 and carbons C-8 and C-7. In some cases, examples of the products formed by isomerization may be codeinone, codeine, morphine, or morphinone. In some examples, the rearrangement that generates the desired isomer occurs spontaneously. In other examples, the rearrangement that generates the desired isomer is promoted by factors such as pH and solvent. In other examples, the carbon-carbon double bond is transposed by contact with a protein or enzyme. The isomerization conversion step is provided in FIG. 4 and represented generally in Scheme 3. R₁, R₂, R₃, and R₄ may be 0, OH, H, CH₃, or other appropriate alkyl groups.

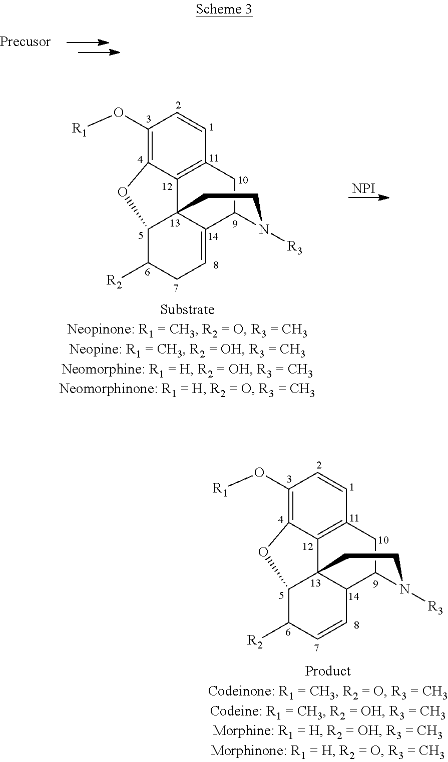

In some examples, the first enzyme that generates an isomer precursor morphinan alkaloid is thebaine 6-O-demethylase (T6ODM). In some cases, T6ODM O-demethylates the substrate thebaine at the C-6 position. In some examples, the product of this reaction is neopinone. In some examples, the T6ODM may catalyze the O-demethylation reaction within a host cell, such as an engineered host cell, as described herein.

In some examples, the isomer precursor morphinan alkaloid is neopinone. In some examples, neopinone undergoes isomerization to codeinone. In some examples, partitioning from neopinone to codeinone may reach equilibrium in aqueous solution such that neopinone and codeinone exist at steady state concentrations. In some examples, the rate of conversion of neopinone to codeinone is promoted by pH. In some examples, the rearrangement of neopinone to codeinone is catalyzed by an enzyme with neopinone isomerase activity. In some examples, this enzyme is a Bet v 1-fold protein. In some examples, this enzyme is a neopinone isomerase (NPI). In some examples, this enzyme is an engineered protein with a truncation of its N-terminal sequence. In some examples, the NPI may catalyze the isomerization reaction within a host cell, such as an engineered host cell, as described herein.

In some examples, the enzyme that acts on codeinone is codeinone reductase (COR). In some cases, COR reduces the ketone at position C-6 of codeinone to form a hydroxyl. In some examples, the product of this reaction is codeine. In some examples, COR is selected from numerous gene duplication and alternative splicing isoforms to exhibit the highest activity when paired with the protein encoding the neopinone isomerase activity. In some examples, the COR may catalyze the reduction reaction within a host cell, such as an engineered host cell, as described herein.

In some examples, the enzyme that acts on codeinone is morphinone reductase (morB). In some cases, morB saturates the carbon-carbon double bond between C-7 and C-8 of codeinone. In some examples, the product of this reaction is hydrocodone. In some examples, the morB may catalyze the reduction reaction within a host cell, such as an engineered host cell, as described herein.

In some examples, the thebaine 6-O-demethylase enzyme may be T6ODM or a T6ODM-like enzyme from plants in the Ranunculales order that biosynthesize morphine, for example Papaver somniferum. In some examples, T6ODM may be a T6ODM-like enzyme from plants that biosynthesize benzylisoquinoline alkaloids, for example P. bracteatum, P. rhoeas, P. nudicaule, and P. orientale. In some examples, the plant enzyme is a 2-oxoglutarate/Fe(II)-dependent dioxygenase that uses 2-oxoglutarate and oxygen and generates succinate and carbon dioxide when demethylating thebaine to produce neopinone. In some examples, T6ODM can also demethylate oripavine to generate neomorphinone.

In other examples, the enzyme with thebaine 6-O-demethylase activity may be from mammals or another vertebrate or invertebrate that biosynthesizes endogenous morphinan alkaloids.

In some examples, the neopinone isomerase (NPI) enzyme may be a Bet v 1-fold protein from plants in the Ranunculales order that biosynthesize morphine, for example Papaver somniferum. In some examples, NPI may be a NPI-like enzyme from plants that biosynthesize benzylisoquinoline alkaloids, for example P. bracteatum, P. rhoeas, P. nudicaule, and P. orientale. In some examples, the Bet v 1 protein includes the following domains in order from the N-terminus to the C-terminus: a β-strand, one or two α-helices, six β-strands, and one or two α-helices. In some examples, a truncation is performed at the N-terminus of the enzyme to remove all or part of the first domain. In some examples, the enzyme may have one or more activity-increasing components as discussed herein and as described in Examples 6 and 7. In some examples, the protein is organized such that it has a Bet v 1 fold and an active site that accepts large, bulky, hydrophobic molecules, such as the morphinan alkaloids. In some examples, the protein may be any plant Bet v 1 protein, pathogenesis-related 10 protein (PR-10), a major latex protein (MLP), fruit or pollen allergen, plant hormone binding protein (e.g., binding to cytokinin or brassinosteroids), plant polyketide cyclase-like protein, or norcoclaurine synthase (NCS)-related protein that has a Bet v 1 fold. In some examples, the function of the Bet v 1-fold protein is to catalyze a reaction that can also occur spontaneously.

In other examples, the enzyme with neopinone isomerase activity may be from mammals or another vertebrate or invertebrate that biosynthesizes endogenous morphinan alkaloids.

In some examples, the codeinone reductase enzyme may be COR or a COR-like enzyme from plants in the Ranunculales order that biosynthesize morphine, for example P. somniferum. In some examples, COR may be a COR-like enzyme from plants that biosynthesize benzylisoquinoline alkaloids, for example P. bracteatum, P. rhoeas, P. nudicaule, and P. orientale. In some examples, the plant enzyme is an oxidoreductase that uses NADPH as a cofactor in the reversible reduction of codeinone to codeine. In some examples, the COR enzyme is a particular gene duplication or splicing variant selected to have select kinetic parameters, for example a higher rate of activity for one or more reactions (K_{cat}), improved binding affinity to one or more substrates (K_{M}), enhanced specificity for substrate codeinone over neopinone, or enhanced thermostability. In some examples, the COR enzyme may act to reduce other morphinan alkaloid substrates, for example neopinone, morphinone, neomorphinone, hydrocodone, hydromorphone, oxycodone, oxymorphone, 14-hydroxycodeinone, or 14-hydroxymorphinone. In some examples, the products of COR activity are neopine, morphine, neomorphine, dihydrocodeine, dihydromorphine, oxycodol, oxymorphol, 14-hydroxcodeine, or 14-hydroxymorphine.

In some examples, the morphinone reductase enzyme may be morB or a morB-like enzyme from bacteria in the Pseudomonas genus. In some examples, morphinone reductase may be an alkene reductase enzyme from a gram-negative bacterium. In some examples, the bacterial enzyme is a α/β-barrel flavoprotein that uses NADH and FMN as cofactors to saturate the carbon-carbon double bond between C-7 and C-8 of codeinone. In some examples, the morB enzyme has select kinetic parameters, for example a higher rate of activity for one or more reactions (Kₑₐₜ), improved substrate binding affinity for one or more substrates (K_{M}), enhanced specificity for one substrate, or enhanced thermostability. The morB enzyme may also reduce other morphinan substrates, for example morphinone, neomorphinone, codeine, morphine, neopine, neomorphine, 14-hydroxycodeinone, or 14-hydroxymorphinone. Examples of products of morB activity are hydromorphone, dihydrocodeine, dihydromorphine, oxycodone, or oxymorphone.

In other examples, combinations of the above enzymes together with additional accessory proteins may function in the production of select morphinan alkaloid isomers. In some examples, these enzymes catalyze the reactions within a host cell, such as an engineered host cell, described herein.

Examples of amino acid sequences for neopinone isomerase activity are set forth in Table 3. An amino acid sequence for a neopinone isomerase that is utilized in converting a precursor morphinan alkaloid with a carbon-carbon double bond between carbons C-14 and C-8 into a product morphinan alkaloid with a carbon-carbon double bond between carbons C-8 and C-7 may be 50% or more identical to a given amino acid sequence as listed in Table 3. For example, an amino acid sequence for such a neopinone isomerase may comprise an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases, an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the nucleic acid sequence encoding the amino acid sequence is altered such as to optimize codon usage for the host organism, for example.

An engineered host cell may be provided that produces a thebaine 6-O-demethylase, neopinone isomerase, and codeinone reductase that converts a precursor morphinan alkaloid isomer into a desired product morphinan alkaloid isomer by rearrangement of a carbon-carbon double bond between carbons C-14 and C-8 and carbons C-8 and C-7, wherein the neopinone isomerase comprises an amino acid sequence selected from the group consisting of SEQ ID NOs: 54, 55, 56, 57, and 58. In some cases, the neopinone isomerase may physically interact with one or more pathway enzymes. In some cases, the physical interaction may change the activity of the one or more pathway enzymes. In some cases, the neopinone isomerase may form a fusion protein with one or more other enzymes. Enzymes that are produced within the engineered host cell may be recovered and purified so as to form a biocatalyst. These one or more enzymes may also be used to catalyze the conversion of a precursor morphinan alkaloid with a carbon-carbon double bond between carbons C-14 and C-8 into a product morphinan alkaloid with a carbon-carbon double bond between carbons C-8 and C-7.

In other examples, the neopinone isomerase comprises an amino acid sequence selected from the group consisting of SEQ ID NOs: 43, 44, 45, 46, 47, 48, 49, 50, 51, 52, 53, 54, 55, 56, 57, and 58 as listed in Table 3.

Examples of amino acid sequences for codeinone reductase activity are set forth in Table 4. An amino acid sequence for a codeinone reductase that is utilized in reducing a ketone at the C-6 position of a morphinan alkaloid to a hydroxyl at that position may be 50% or more identical to a given amino acid sequence as listed in Table 4. For example, an amino acid sequence for such a codeinone reductase may comprise an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases, an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the nucleic acid sequence encoding the amino acid sequence is altered such as to optimize codon usage for the host organism, for example.

An engineered host cell may be provided that produces a thebaine 6-O-demethylase, neopinone isomerase, and codeinone reductase that converts a precursor morphinan alkaloid isomer into a desired product morphinan alkaloid isomer by rearrangement of a carbon-carbon double bond between carbons C-14 and C-8 and carbons C-8 and C-7 and reduction of a ketone at the C-6 position to a hydroxyl, wherein the codeinone reductase comprises an amino acid sequence selected from the group consisting of SEQ ID NOs: 59, 60, 61, 62, 63, 64, 65, 66, 67, and 68 as listed in Table 4. In some cases, the codeinone reductase may interact with or form a fusion protein with other enzymes. The enzymes that are produced within the engineered host cell may be recovered and purified so as to form a biocatalyst. These one or more enzymes may also be used to catalyze the conversion of a precursor morphinan alkaloid with a carbon-carbon double bond between carbons C-14 and C-8 into a product morphinan alkaloid with a carbon-carbon double bond between carbons C-8 and C-7.

In additional cases, the one or more enzymes that are recovered from the engineered host cell may be used in a process for converting a precursor morphinan alkaloid with a carbon-carbon double bond between carbons C-14 and C-8 into a product morphinan alkaloid with a carbon-carbon double bond between carbons C-8 and C-7. The process may include contacting the precursor morphinan alkaloid isomer with the recovered enzymes in an amount sufficient to convert said precursor morphinan alkaloid isomer to the desired morphinan alkaloid isomer product. In some examples, the precursor morphinan alkaloid isomer may be contacted with a sufficient amount of the one or more enzymes such that at least 5% of said precursor morphinan alkaloid isomer is converted to the desired product morphinan alkaloid isomer. In further examples, the precursor morphinan alkaloid isomer may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said precursor morphinan alkaloid isomer is converted to the desired product morphinan alkaloid isomer.

In some examples, process conditions are implemented to support the formation of the desired product morphinan alkaloid isomer in engineered host cells. In some cases, engineered host cells are grown at pH 3.3, and once high cell density is reached the pH is adjusted to pH 6-6.5 to support continued production of the desired product morphinan alkaloid isomers at higher pH. In some cases, the engineered host cells produce additional enzymes to convert sugar and other simple starting materials, such as tyrosine, to the desired product morphinan alkaloid isomers.

In some examples, one or more of the enzymes converting a precursor morphinan alkaloid with a carbon-carbon double bond between carbons C-14 and C-8 to a product morphinan alkaloid with a carbon-carbon double bond between carbons C-8 and C-7 are localized to cellular compartments. In some examples, T6ODM, COR or morB, and NPI may be modified such that they encode targeting sequences that localize them to the endoplasmic reticulum membrane of the engineered host cell. In particular, in certain instances, the host cell may be engineered to increase production of product morphinan alkaloid isomers or its precursors by localizing NPI and/or T6ODM and/or COR and/or morB to organelles in the yeast cell. NPI and/or T6ODM and/or COR and/or morB may be localized to the yeast endoplasmic reticulum in order to decrease the spatial distance between these enzymes. By increased production is meant both the production of some amount of the compound of interest where the control has no production of the compound of interest, as well as an increase of 10% or more, such as 50% or more, including 2-fold or more, e.g., 5-fold or more, such as 10-fold or more in situations where the control has some production of the compound of interest.

In other examples, T6ODM and NPI may be co-localized to a single protein fusion. In other examples, COR or morB and NPI may be co-localized to a single protein fusion. In some examples, the fusion is between the proteins is created by one of several methods, including, direct fusion, co-localization to a yeast organelle, or by enzyme co-localization tools such as leucine zippers, protein scaffolds that utilize adaptor domains, or RNA scaffolds that utilize aptamers. Co-localizing the neopinone isomerase enzyme may facilitate substrate channeling between the active sites of the enzymes and limit the diffusion of unstable intermediates such as neopinone and codeinone.

In some examples, an engineered T6ODM enzyme is used in converting between morphinan alkaloid isomers. In some examples, a T6ODM enzyme is engineered to combine two functions: (1) the O-demethylation of thebaine at the C-6 position, and (2) the rearrangement of a carbon-carbon double bond between carbons C-14 and C-8 and carbons C-8 and C-7.

In some examples, an enzyme with thebaine 6-O-demethylase activity is fused to a peptide with a Bet v 1 fold. In some examples, the thebaine 6-O-demethylase enzyme and the Bet v 1 fold protein may be fused in any order from N-terminus to C-terminus, C-terminus to N-terminus, N-terminus to N-terminus, or C-terminus to C-terminus. In some examples, the two protein sequences may be fused directly or fused through a peptide linker region.

In some examples, an enzyme with thebaine 6-O-demethylase activity is fused to a peptide with a Bet v 1 fold by circular permutation. In some cases, the N- and C-termini of T6ODM are fused and the Bet v 1 sequence is then inserted randomly within this sequence. In some cases, the resulting fusion protein library is screened for production of the desired morphinan alkaloid isomer product. In other cases, a circular permutation T6ODM library is first screened for activity in the absence of Bet v 1. In other cases, the N- and C-termini of T6ODM are fused and the enzyme is digested and blunt end cloned. In other cases, this library of circularly permuted T6ODM is screened for thebaine 6-O-demethylase activity. In other cases, active variants from the circularly permuted T6ODM library are then used to design protein fusions with a peptide with a Bet v 1 fold.

In some examples, an engineered COR or morB enzyme is used in converting between morphinan alkaloid isomers. In some examples, a COR or morB enzyme is engineered to combine two functions: (1) the rearrangement of a carbon-carbon double bond between carbons C-14 and C-8 and carbons C-8 and C-7, and (2) the reduction of a morphinan alkaloid isomer product.

In some examples, an enzyme with opioid reductase activity is fused to a peptide with a Bet v 1 fold. In some examples, the COR or morB enzyme and the Bet v 1 fold protein may be fused in any order from N-terminus to C-terminus, C-terminus to N-terminus, N-terminus to N-terminus, or C-terminus to C-terminus. In some examples, the two protein sequences may be fused directly or fused through a peptide linker region.

In some examples, an enzyme with opioid reductase activity is fused to a peptide with a Bet v 1 fold by circular permutation. In some cases, the N- and C-termini of COR or morB are fused and the Bet v 1 sequence is then inserted randomly within this sequence. In some cases, the resulting fusion protein library is screened for production of the desired morphinan alkaloid isomer product. In other cases, a circular permutation COR or morB library is first screened for activity in the absence of Bet v 1. In other cases, the N- and C-termini of COR or morB are fused and the enzyme is digested and blunt end cloned. In other cases, this library of circularly permuted COR or morB is screened for opioid reductase activity. In other cases, active variants from the circularly permuted COR or morB library are then used to design protein fusions with a peptide with a Bet v 1 fold.

The one or more enzymes that may be used to convert a precursor morphinan alkaloid with a carbon-carbon double bond between carbons C-14 and C-8 to a product morphinan alkaloid with a carbon-carbon double bond between carbons C-8 and C-7 may contact the precursor morphinan alkaloid isomer in vitro. Additionally, or alternatively, the one or more enzymes that may be used to convert a precursor morphinan alkaloid with a carbon-carbon double bond between carbons C-14 and C-8 to a product morphinan alkaloid with a carbon-carbon double bond between carbons C-8 and C-7 may contact the precursor morphinan alkaloid isomer in vivo. Additionally, the one or more enzymes that may be used to convert a precursor morphinan alkaloid with a carbon-carbon double bond between carbons C-14 and C-8 to a product morphinan alkaloid with a carbon-carbon double bond between carbons C-8 and C-7 may be provided to a cell having the precursor morphinan alkaloid isomer within, or may be produced within an engineered host cell.

In some examples, the methods provide for engineered host cells that produce an alkaloid product, wherein the conversion of a precursor morphinan alkaloid with a carbon-carbon double bond between carbons C-14 and C-8 to a product morphinan alkaloid with a carbon-carbon double bond between carbons C-8 and C-7 may comprise a significant step in the production of an alkaloid product. In some examples, the alkaloid product is a codeinone. In still other embodiments, the alkaloid product is derived from a codeinone, including for example, downstream morphinan alkaloids. In another embodiment, a precursor morphinan alkaloid with a carbon-carbon double bond between carbons C-14 and C-8 is an intermediate toward the product in of the engineered host cell. In still other embodiments, the alkaloid product is selected from the group consisting of morphinan, nor-opioid, or nal-opioid alkaloids.

In some examples, the substrate of the O-demethylation reaction is a compound of Formula VI:

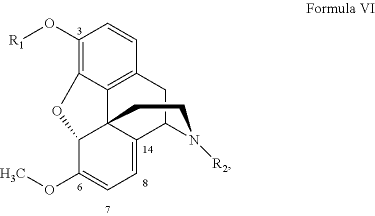

or a salt thereof, wherein:
R₁, and R₂ are independently selected from hydrogen and methyl.

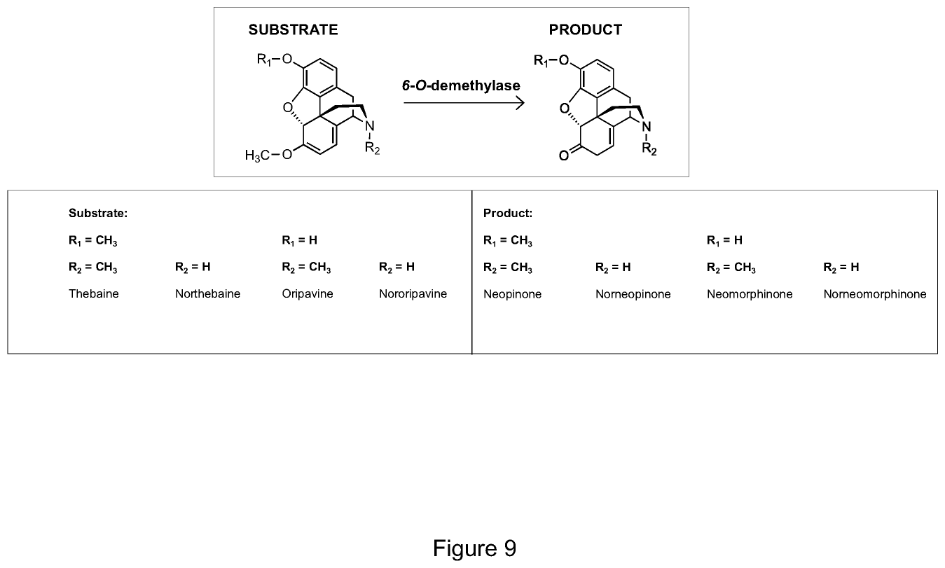

In some other examples, R₁ and R₂ are methyl, and the O-demethylation reaction is catalyzed by a thebaine 6-O-demethylase. Other examples of 6-O-demethylation reactions are provided in FIG. 9.

In some examples, the substrate of the isomerization reaction is a compound of Formula VII:

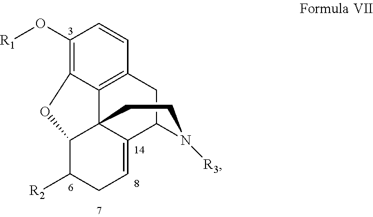

or a salt thereof, wherein:
R₁, and R₃ are independently selected from hydrogen and methyl, and R₂ is independently selected from hydroxyl and oxygen.

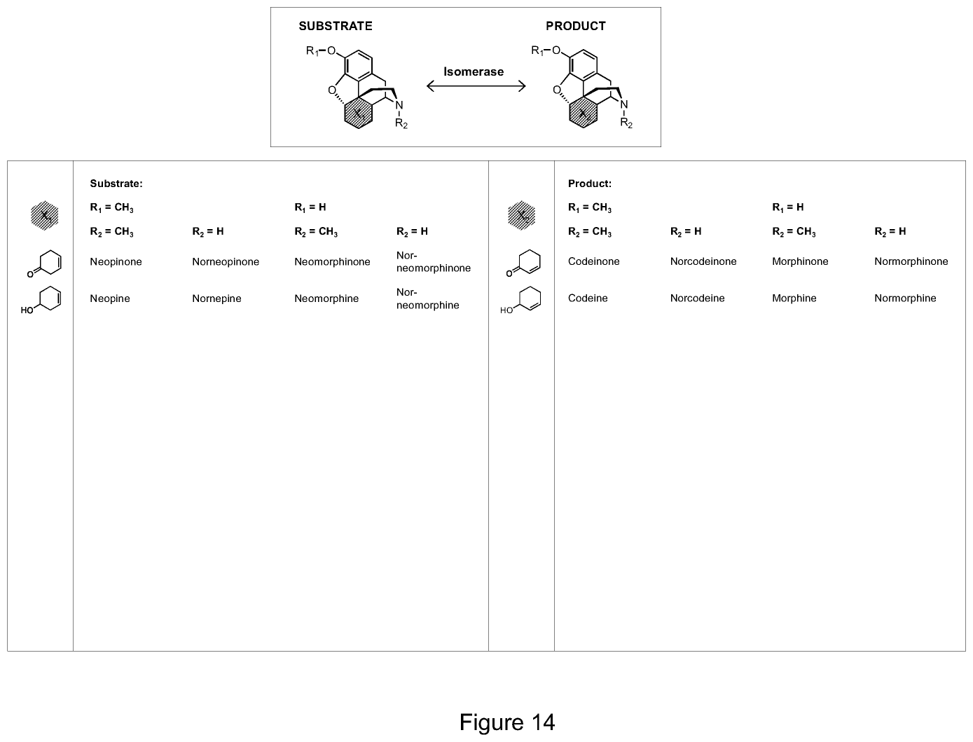

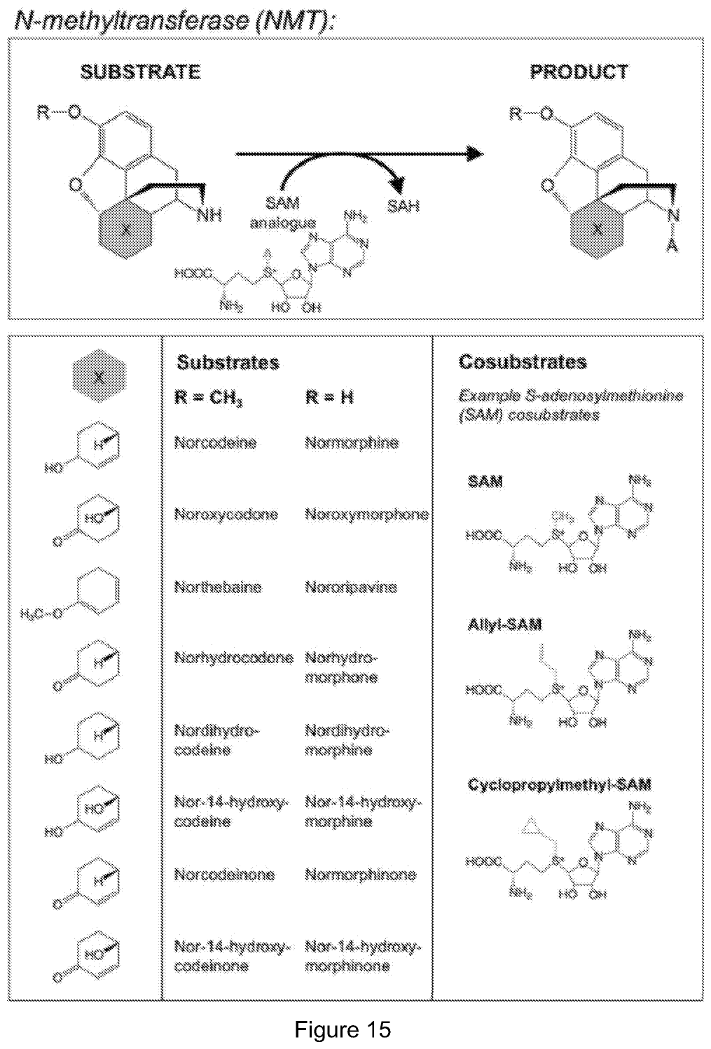

In some other examples, R₁, and R₃ are methyl and R₂ is oxygen, and the isomerization reaction is catalyzed by a neopinone isomerase. Other examples of isomerization reactions are provided in FIG. 14.

In some examples, the substrate of the reduction reaction is a compound of Formula VIII:

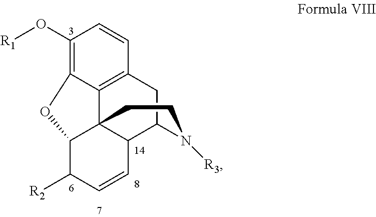

or a salt thereof, wherein:
R₁, and R₃ are independently selected from hydrogen and methyl; and R₂ is independently selected from hydroxyl and oxygen.

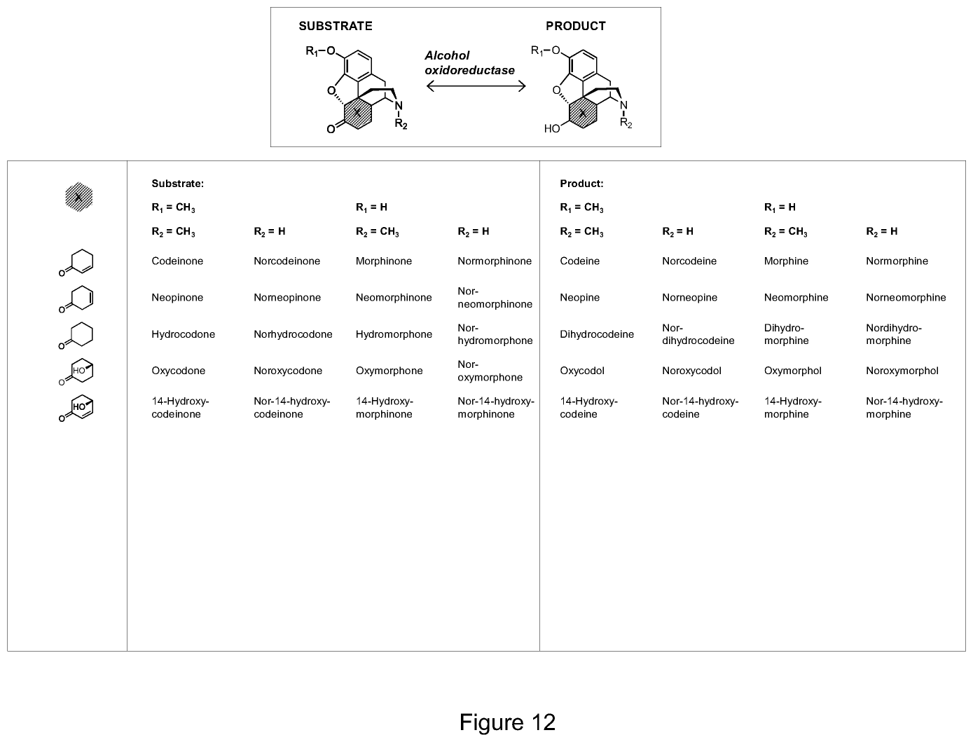

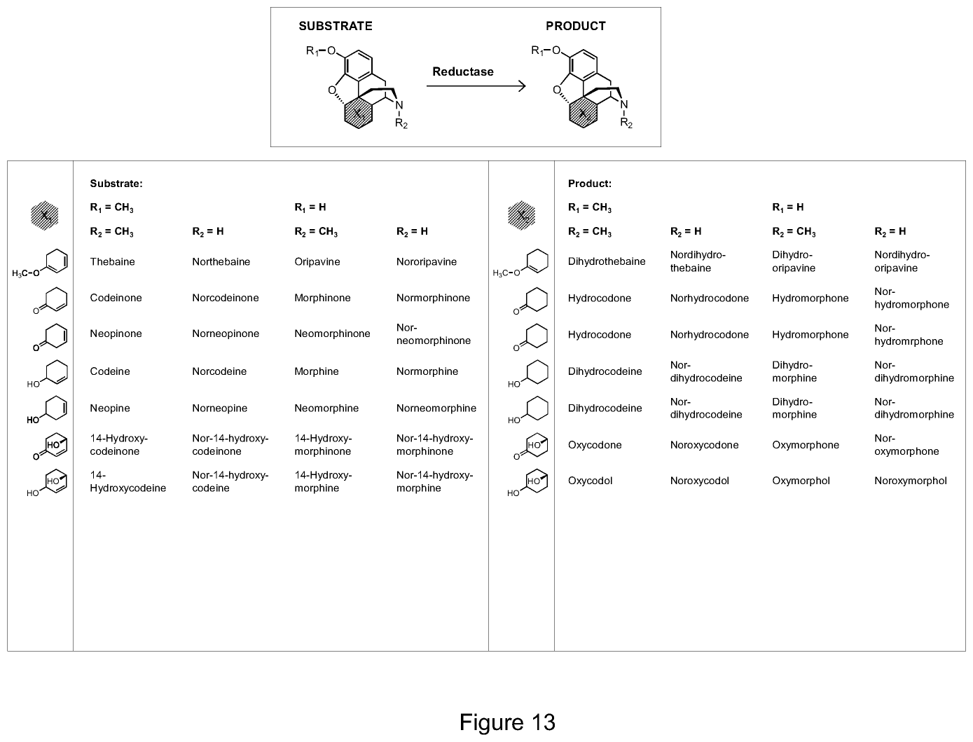

In some other examples, R₁ and R₃ are methyl and R₂ is oxygen, and the reduction reaction is catalyzed by a codeinone reductase. In some other examples, the reduction reaction is catalyzed by a morphinone reductase. Other examples of reduction reactions are provided in FIGS. 12 and 13.

In some examples, the methods provide for engineered host cells that produce morphinan alkaloid products from neopinone. The conversion of neopinone to codeinone may comprise a significant step in the production of diverse morphinan alkaloid products from a simple starting material. In some examples, the simple starting material is L-tyrosine or a sugar (e.g., glucose). The diverse alkaloid products can include, without limitation, morphinan, nor-opioid, or nal-opioid alkaloids.

In some examples, the engineered host cells are grown through a fed-batch fermentation process in which the simple starting material is fed over time and converted to the precursor morphinan alkaloid continuously over time in the engineered host cell, thereby providing a constant source of the precursor morphinan alkaloid. In some examples, the continuous source of precursor morphinan alkaloid is isomerized to the product morphinan alkaloid isomer continuously over time and then converted to the downstream alkaloid product through one or more enzymes that act on the morphinan alkaloid isomer in the engineered host cell, thereby providing a constant pull of the product isomer to the downstream alkaloid product. In some examples, the dynamic system process (e.g., continuous supply of the precursor morphinan alkaloid and continuous conversion of the product morphinan alkaloid isomer to a downstream alkaloid product) is a beneficial component to achieving increased production of desired alkaloid products through an enhanced reversible isomerization reaction.

In some cases, the pairing of a neopinone isomerase with a COR variant exhibiting particular kinetic properties is a beneficial component to achieving increased production of desired alkaloid products in an engineered host cell. In some cases, the pairing of a neopinone isomerase with a morB variant exhibiting particular kinetic properties is a beneficial component to achieving increased production of desired alkaloid products in an engineered host cell.

Any suitable carbon source may be used as a starting material toward a morphinan alkaloid. Suitable precursors can include, without limitation, simple starting materials such as monosaccharides (e.g., glucose, fructose, galactose, xylose), oligosaccharides (e.g., lactose, sucrose, raffinose), polysaccharides (e.g., starch, cellulose), or a combination thereof. In some examples, unpurified mixtures from renewable feedstocks can be used (e.g., cornsteep liquor, sugar beet molasses, barley malt, biomass hydrolysate). In still other embodiments, the carbon precursor can be a one-carbon compound (e.g., methanol, carbon dioxide) or a two-carbon compound (e.g., ethanol). In yet other embodiments, other carbon-containing compounds can be utilized, for example, methylamine, glucosamine, and amino acids (e.g., L-tyrosine).

In some examples, the benzylisoquinoline alkaloid product, or a derivative thereof, is recovered. In some examples, the benzylisoquinoline alkaloid product is recovered from a cell culture. hn some examples, the benzylisoquinoline alkaloid product is a morphinan, nor-opioid, or nal-opioid alkaloid.

TABLE 3
Example amino acid sequences of morphinan alkaloid isomerizing enzymes.

| Sequence | | | SEQ. ID. |
|---|---|---|---|
| Name | Description | Sequence | NO. |
| THS | | | |
| | P. bracteatum | MAPRGVSGLVGKLSTELDVNCDAEKYYNMYKNGEDVQKAVPHLC | SEQ. ID. |
| | | MDVKVISGDATRSGCIKEWNVNIDGKTIRSVEETTHNDETKTLRH | NO. 35 |
| | | RVFEGDMMKDYKKFDTIMEVNPKPDGNGCVVTRSIEYEKVNENSP | |
| | | TPFDYLQFGHQAMEDMNKY | |
| | P. setigerum | MLVGKLSTELEVDCDAEKYYNMYKHGEDVKKALCVDVKVISGDPT | SEQ. ID. |
| | | RSGCIKEWNVNIDGKTIRSVEETTHNDETKTLRHRVFEGDMMKDF | NO. 36 |
| | | KKFDTIMVVNPKPDGNGCVVTRSIEYEKTNENSPTPFDYLQFGHQ | |
| | | AIEDMNKYL | |
| | P. setigerum | MLVGKLSTELEVDCDAEKYYNMYKHGEDKRQCVDVKVISGDPTRS | SEQ. ID. |
| | | GCIKEWNVNIDGKTIRSVEETTHNDETKTLRHRVFEGDMMKDFK | NO. 37 |
| | | KFDTIMVVNPKPDGNGCVVTRSIEYEKTNENSPTPFDYLQFGHQAI | |
| | | EDMNKY | |
| | P. setigerum | MLVGKLSTELEVDCDAEKYYNMYKHGEDVKKAVPHLCVDVKIISG | SEQ. ID. |
| | | DPTSSGCIKEWNVNIDGKTIRSVEETTHDDETKTLRHRVFEGDVM | NO. 38 |
| | | KDFKKFDTIMVVNPKPDGNGCVVTRSIEYEKTNENSPTPFDYLQFG | |
| | | HQAIEDMNKYL | |
| | P. setigerum | MVKIISGDPTSSGCIKEWNVNIDGKTIRSVEETTHDDETKTLRHRV | SEQ. ID. |
| | | FEGDVMKDFKKFDTIMVVNPKPDGNGCVVTRSIEYEKTNENSPTP | NO. 39 |
| | | FDYLQFGHQAIEDMNKYL | |
| | P. somniferum | MDSINSSIYFCAYFRELIIKLLMAPPGVSGLVGKLSTELEVNCDAEK | SEQ. ID. |
| | | YYNMYKHGEDVQKAVPHLCVDVKVISGDPTRSGCIKEWNVNIDGK | NO. 40 |
| | | TIRSVEETTHNDETKTLRHRVFEGDVMKDFKKFDTIMVVNPKPDG | |
| | | NGCVVTRSIEYEKTNDNSPTPFDYLQFGHQAIEDMNKYLRDSE | |
| | P. somniferum | MNFFIKDHLYICLVGKLSTELEVDCDAEKYYNMYKHGEDVKKAVP | SEQ. ID. |
| | | HLCVDVKIISGDPTSSGCIKEWNVNIDGKTIRSVEETTHDDETKTLR | NO. 41 |
| | | HRVFEGDVMKDFKKFDTIMVVNPKPDGNGCVVTRSIEYEKTNENS | |
| | | PTPFDYLQFGHQAIEDMNKYLRDSESN | |
| | P. somniferum | MAPLGVSGLVGKLSTELEVDCDAEKYYNMYKHGEDVKKAVPHLC | SEQ. ID. |
| | | VDVKIISGDPTSSGCIKEWNVNIDGKTIRSVEETTHDDETKTLRHR | NO. 42 |
| | | VFEGDVMKDFKKFDTIMVVNPKPDGNGCVVTRSIEYEKTNENSPT | |
| | | PFDYLQFGHQAIEDMNKYLRDSESN | |
| NPI | | | |
| | P. setigerum | MAQNGDFGIVGKLVIELEVSSPADKFYTIFKHQKDVPKAIPHLFTD | SEQ. ID. |
| | | GKVIEGDARRSGCIKEWKYVLEGKTISVTEKTTHNDETKTLHHRIF | NO. 43 |
| | | EGDLMKDYKKFDSIIEVNPKPTGHGSIVTWSFVYEKINKNSPTPFA | |
| | | YLPFCYQAIEDINNHLAASE | |
| | P. setigerum | MAHHGVSGLVGKLVTQLEVNCDADKLYKIYVPKAISHLFTGVKVLE | SEQ. ID. |
| | | GHGLRSGCIKEWKYIIDGKALTAVEETTHGDETRTLKHRVIDGDL | NO. 44 |
| | | MKDYKKFEKIIEANPKPNGHGSIVTVSLLYEKINEDSPAPFDHLKFF | |
| | | HQNIEDMNSHICASE | |
| | P. setigerum | MARHSVSGLVGKLVTELEVSSDAEKYYKVYKHAEDVEKAIPHLCT | SEQ. ID. |
| | | GIRVIKGEASRSGCIKEWNFILEGKAIRSIEETTHNDATRTVHHRIFE | NO. 45 |
| | | GNLMKDYKKFDSIIEVNGCIVARSIVYEKRSEDSPTPFAYILFCHQAI | |
| | | EDMNKHLCDNE | |
| | P. setigerum | MDSVSAALVFHSSIYLCAMAHHGVSGLVGKIVTELEVNCNADEFYK | SEQ. ID. |
| | | ILKRDEDVPRAVSDLFPPVKIAKGDGLVSGCIKEWDCVLDGKAMSG | NO. 46 |
| | | KEETTHNDETRTLRHRELKKFDSIIEVNPKPNGHGSIVTWSIEYEK | |
| | | MNEDSPAPFAYLASFHQPNGHGSIVTWSIEYEKMNEDSPAPFAYL | |
| | | ASFHQNVVEVDSHLCLSE | |
| | P. bracteatum | MYSVSAALVSIAPYTFVRTDNNLRLLMACDGVSGLVGKLVTELKV | SEQ. ID. |
| | | NCDADKYYQIYKRPDDLQKAIPHLCTGIKLINGDASRSGCIKEWNF | NO. 47 |
| | | TLEGKRIHTVEETTHNDETRTLHHRIFEGDLMKDYKKFDSIIEVNP | |
| | | KPNGNGCVVKRSIVYEKINKDSPTPFSYLPFCHQAIEDMNKHLCDS | |
| | | E | |
| | P. bracteatum | MACDGVSGLVGKLVTELKVNCDADKYYQIYKRPDDLQKAIPHLCT | SEQ. ID. |
| | | GIKLINGDASRSGCIKEWNFTLEGKRIHTVEETTHNDETRTLHHRI | NO. 48 |
| | | FEGDLMKDYKKFDSIIEVNPKPNGNGCVVKRSIVYEKINKDSPTPFS | |
| | | YLPFCHQAIEDMNKHLCDSE | |
| | P. bracteatum | MAHHGVSGLVGKLVTQLEVNCDADEFYKIWKHHEEVPQAVSHLF | SEQ. ID. |
| | | PAVKVVKGDGLVSGCIKEWDYILEGKAMSAMEETTHNDETRTLH | NO. 49 |
| | | HRIVEGEVMKDYKAIASIIEVNPNPNGHGSIVTWSIEYEKMNEDSP | |
| | | TPFAYLEFFHQNLVDMNSHLYVGSDSHLHVDE | |
| | P. rhoeas | MAPHGVSDLSGKLVTELEVSCDADKYYKIYKHAEDVQKAVPHLCT | SEQ. ID. |
| | | DVKVINGDATLSGCIKEWHYILEGKALSAKEETTINDETRTLHHRV | NO. 50 |
| | | LEGDMMKDYKKFDSVIEVNPKPNGNGSVVTRSIAYEKINEDSPTPF | |
| | | AYILFSHRAVEDMNKYLCDSE | |
| | P. rhoeas | MAPHGVSDLSGKLVTELEVSCDADKYYKIYKHAEDVQKAVPHLCT | SEQ. ID. |
| | | DVKVINGDATLSGCIKEWHYILEGKALSAKEETTINDETRTLHHRV | NO. 51 |
| | | LEGDMMKDYKKFDSVIEVNPKPNGNGSVVTRSIAYEKINEDPAPFA | |
| | | YLAFFHQNAVEVNSYLCLSE | |
| | P. rhoeas | MAPHGVSDLSGKLVTELEVSCDADKYYKIYKHAEDVQKAVPHLCT | SEQ. ID. |
| | | DVKVINGDATLSGCIKEWHYILEGKALSAKEETTINDETRTLHHRV | NO. 52 |
| | | LEGDMMKDYKKFDSVIEVNPKPNGNGSVVTRSIAYEKINEDSSSLC | |
| | | VSSFLPSERG | |
| | P. rhoeas | MAHHGVSGLVGKLVTQLEVNCDADKFYKMAKHHEDVPKAVPHFF | SEQ. ID. |
| | | TAVKVTEGDGLVSGCIKEWDYILEGKAMSCKEEEETTHYDETRTL | NO. 53 |
| | | HHRVFGGDMMMDYKKFDAIIEVNPKPNVHGCIVTWSIAYEKINED | |
| | | SPVPFDYLAFYHQNIIDVGSHLCSE | |
| | P. somniferum | MDSVSAALVFHSSIYLCAMAHHGVSGLVGKIVTELEVNCNADEFYK | SEQ. ID. |
| | | ILKRDEDVPRAVSDLFPPVKIAKGDGLVSGCIKEWDCVLDGKAMSG | NO. 54 |
| | | KEETTHNDETRTLRHRELEGDLMKDYKKFDSIIEVNPKPNGHGSIV | |
| | | TWSIEYEKMNEDSPAPFAYLASFHQNVVEVDSHLCLSE | |
| | P. somniferum | MAHHGVSGLVGKIVTELEVNCNADEFYKILKRDEDVPRAVSDLFPP | SEQ. ID. |
| | | VKIAKGDGLVSGCIKEWDCVLDGKAMSGKEETTHNDETRTLRHRE | NO. 55 |
| | | LEGDLMKDYKKFDSIIEVNPKPNGHGSIVTWSIEYEKMNEDSPAPF | |
| | | AYLASFHQNVVEVDSHLCLSE | |
| | P. somniferum | MAHHGISGLVGKLVTQLEVNCDADEFYKIWKHHEEVPKAVSHLLP | SEQ. ID. |
| | | AVKVVKGDGLVSGCIKEWHYILEGKAMSAMEETTHNDETRTLHH | NO. 56 |
| | | QVVEGELMKDYKAIASIIQVNPNGSIVTWSIEYEKMNEDSPTPFAY | |
| | | LEFFHQNIIDMNSHLYVGSDSHLHVDE | |
| | P. somniferum | MAHHGVSGLVGKLVTELEVHCNADAYYKIFKHQEDVPKAMPHLY | SEQ. ID. |
| | | TGGKVISGDATRSGCIKEWNYILEGKALIAVEETTHDDETRTLTHRI | NO. 57 |
| | | TGGDLTKDYKKFVKIVEVNPKPNGHGSIVTVSLVYEKMNEGSPTPF | |
| | | NYLQFVHQTIVGLNSHICAS | |
| | P. somniferum | MAHHGISGLVGKLVIGLEVNCDADKYYQIFKHAEDVQKAVPHHYD | SEQ. ID. |
| | | SIKVINGDAKSSGCIKEWNFIHEGKTFHTVEETTHNDETRTLHHRI | NO. 58 |
| | | FEGDLMKDYKKFDLIIEANPKPTGNGCVVTWTIEYEKINQDSPAPI | |
| | | AYLPFCNQVIEDMNKHLCDSE | |

TABLE 4
Example amino acid sequences of morphinan alkaloid reducing enzymes.

| Sequence | | | SEQ. ID. |
|---|---|---|---|
| Name | Description | Sequence | NO. |
| COR | | | |
| | P. somniferum | MESNGVPMITLSSGIRMPALGMGTAETMVKGTEREKLAFLKAI | SEQ. ID. |
| | COR 1.3 | EVGYRHFDTAAAYQSEECLGEAIAEALQLGLIKSRDELFITSKL | NO. 59 |
| | | WCADAHADLVLPALQNSLRNLKLDYLDLYLIHHPVSLKPGKFV | |
| | | NEIPKDHILPMDYKSVWAAMEECQTLGFTRAIGVCNFSCKKLQ | |
| | | ELMAAAKIPPVVNQVEMSPTLHQKNLREYCKANNIMITAHSVL | |
| | | GAICAPWGSNAVMDSKVLHQIAVARGKSVAQVSMRWVYQQGA | |
| | | SLVVKSFNEGRMKENLKIFDWELTAENMEKISEIPQSRTSSADF | |
| | | LLSPTGPFKTEEEFWDEKD | |
| | P. somniferum | MESNGVPMITLSSGIRMPALGMGTVETMEKGTEREKLAFLKAI | SEQ. ID. |
| | | EVGYRRFDTAAAYQTEECLGEAIAEALQLGLIKSRDELFITSKL | NO. 60 |
| | | WCADAHADLVLPALQNSLRNLKLDYLDLYLIHHPVSLKPGKFV | |
| | | NEIPKDHILPMDYKSVWAAMEECQTLGFTRAIGVCNFSCKKLQ | |
| | | ELMATANSPPVVNQVEMSPTLHQKNLREYCKANNIMITAHSV | |
| | | LGAIGAPWGSNAVMDSKVLHQIAVARGKSVAQVSMRWVYQQG | |
| | | ASLVVKSFNEARMKENLKIFDWELTAEDMEKISEIPQSRTSSAA | |
| | | FLLSPTGPFKTEEEFWDEKD | |
| | P. somniferum | MESNGVPMITLSSGIRMPALGMGTAETMVKGTEREKLAFLKAI | SEQ. ID. |
| | | EVGYRHFDTAAAYQSEECLGEAIAEALQLGLIKSRDELFITSKL | NO. 61 |
| | | WCADAHADLVLPALQNSLRNLKLDYLDLYLIHHPVSLKPGKFV | |
| | | NEIPKDHILPMDYKSVWAAMEECQTLGFTRAIGVCNFSCKKLQ | |
| | | ELMATANSPPVVNQVEMSPTLHQKNLREYCKANNIMITAHSV | |
| | | LGAVGAAWGTNAVMHSKVLHQIAVARKSVAQVSMRWVYQQG | |
| | | ASLVVKSFNEARMKEDLKIFDWELTAEDMEKISEIPQSRTSSAA | |
| | | FLLSPTGPFKTEEEFWDEKD | |
| | P. somniferum | MESNGVPMITLSSGIRMPALGMGTVETMEKGTEREKLAFLKAI | SEQ. ID. |
| | | EVGYRHFDTAAAYQTEECLGEAIAEALQLGLIKSRDELFITSKL | NO. 62 |
| | | WCADAHADLVLPALQNSLRNLKLDYLDLYLIHHPVSLKPGKFV | |
| | | NEIPKDHILPMDYKSVWAAMEECQTLGFTRAIGVCNFSCKKLQ | |
| | | ELMATANSPPVVNRVEMSPTLHQKNLREYCKANNIMITAHSVL | |
| | | GAVGAAWGTNAVMHSKVLHQIAVARGKSVAQVSMRWVYQQG | |
| | | ASLVVKSFNEARMKENLKIFDWELTAEDMEKISEIPQSRTSSAA | |
| | | FLLSPTGPFKTEEEFWDEKD | |
| | P. somniferum | MESNGVPMITLSSGIRMPALGMGTAETMVKGTEREKLAFLKAI | SEQ. ID. |
| | | EVGYRHFDTAAAYQSEECLGEAIAEALQLGLIKSRDELFITSKL | NO. 63 |
| | | WCADAHADLVLPALQNSLRNLKLEYLDLYLIHHPVSLKPGKFV | |
| | | NEIPKDHILPMDYKSVWAAMEECQTLGFTRAIGVCNFSCKKLQ | |
| | | ELMATANSPPVVNQVEMSPTLHQKNLREYCKANNIMITAHSV | |
| | | LGAVGAAWGTNAVMHSKVLHQIAVARGKSVAQVSMRWVYQQ | |
| | | GASLVVKSFNEARMKENLKIFDWELTAEDVEKISEIPQSRTSSA | |
| | | AFLLSPTGPFKTEEEFWDEKD | |
| | P. somniferum | MESNGVPMITLSSGIRMPALGMGTVETMEKGTEREKLAFLKAI | SEQ. ID. |
| | | EVGYRHFDTAAAYQTEECLGEAIAEALQLGLIKSRDELFITSKL | NO. 64 |
| | | WCADAHADLVLPALQNSLRNLKLDYLDLYLIHHPVSLKPGKFV | |
| | | NEIPKDHILPMDYKSVWAAMEECQTLGFTRAIGVCNFSCKKLQ | |
| | | ELMATANSPPVVNQVEMSPTLHQKNLREYCKANNIMITAHSV | |
| | | LGAVGAAWGTNAVMHSKVLHQIAVARGKSVAQVSMRWVYQQ | |
| | | GASLVVKSFNEARMKENLKIFDWELTAEDMEKISEIPQSRTSSA | |
| | | DFLLSPTGPFKTEEEFWDEKD | |
| | P. setigerum | MESNGVPMITLSSGIRMPALGMGTVETMEKGTEREKLAFLKAI | SEQ. ID. |
| | | EVGYRHFDTAAAYQTEECLGEAIAEALQLGLIKSRDELFITSKL | NO. 65 |
| | | WCADAHADLVLPALQNSLRNLKLDYLDLYLIRHPVSLKPGKFV | |
| | | NEIPKDHILPMDYKSVWAAMEECQTLGFTRAIGVCNFSCKKLQ | |
| | | ELMATVNSPPVVNQVEMSPTLHQKNLREYCKANNIMITAHSVL | |
| | | GAVGAAWGTKAVMHSKVLHQIAVARGKSVAQVSMRWVYQQG | |
| | | ASLVVKSFNEARMKENLKIFDWELTAEDMEKISEIPQSRTSSAA | |
| | | FLLSPTGPFKTEEEFWDEKD | |
| | P. bracteatum | MESNGVPMITLSSGIRMPALGMGTVETMEKGTEREKLAFLKAI | SEQ. ID. |
| | | EVGYRHFDTAAAYQTEECLGEAIAEALQLGLIKSREELFITSKL | NO. 66 |
| | | WCTDAHADLVLPALQNSLRNLKLEYLDLYLIHFPVSLKPGKIVS | |
| | | DIPKDQMLPMDYKSVWVAMEECQTLGFTRAIGVSNFSCKKLQ | |
| | | ELMATANSPPVVNEVEMSPVFQQKNLRAYCKANNIMITAYSVL | |
| | | GARGAAWGSNAVMDSKVLHEIAVARGKSVAQVSMRWVYQQG | |
| | | ACLVVKSFNEERMKENLKIFDWELSAEDMEMISEIPQCRTSSA | |
| | | DFLLSPTGPFKTEEEFWDEKD | |
| | P. somniferum | MESNGVPMITLSSGIRMPALGMGTAETMVKGAEREKLAFLKAI | SEQ. ID. |
| | COR 1.3 Mutant | EVGYRHFDTAAAYQSEECLGEAIAEALQLGLIKSRDELFITSKL | NO. 67 |
| | | WCADAHADLVLPALQNSLRNLKLDYLDLYLIHHPVSLKPGKFV | |
| | | NEIPKDHILPMDYKSVWAAMEECQTLGFTRAIGVCNFSCKKLQ | |
| | | ELMAAAKIPPVVNQVEMSPTLHQKNLREYCKANNIMITAHSVL | |
| | | GAICAPWGSNAVMDSKVLHQIAVARGKSVAQVSMRWVYQQGA | |
| | | SLVVKSFNEGRMKENLKIFDWELTAENMEKISEIPQSRTSSADF | |
| | | LLSPTGPFKTEEEFWDEKD | |
| | P. somniferum | MESNGVPMITLSSGIRMPALGMGTAETMVKGTEREKLAFLKAI | SEQ. ID. |
| | COR 1.3 Mutant | EVGYRHFDTAAAYQSEECLGEAIAEALQLGLIKSRDELFITSKL | NO. 68 |
| | | WCADAHADLVLPALQNSLRNLKLDYLDLYLIHHPVSLKPGKFV | |
| | | NEIPKDHILPMDYKSVWAAMEECQTLGFTRAIGVCNFSCKKLQ | |
| | | ELMAAAKIPPVVNQVEMSPTLHQKNLREYCKANNIMITAHSVL | |
| | | GAICAPWGSNAVMDFKVLHQIAVARGKSVAQVSMRWVYQQG | |
| | | ASLVVKSFNEGRMKENLKIFDWELTAENMEKISEIPQSRTSSAD | |
| | | FLLSPTGPFKTEEEFWDEKD | |

### Benzylquinoline Alkaloid Generating Modifications

Some methods, processes, and systems provided herein describe the conversion of BIA precursors to 1-benzylisoquinoline alkaloids. Some of these methods, processes, and systems may comprise an engineered host cell. In some examples, the production of nococlaurine, or a 1-benzylisoquinoline alkaloid, from 4-HPAA and dopamine, or BIA precursors, is described. In some examples, the production of norlaudonosoline, or a 1-benzylisoquinoline alkaloid, from 3,4-DHPA and dopamine, or BIA precursors, is described. In some examples, the conversion of BIA precursors to 1-benzylisoquinoline alkaloids is a key step in the conversion of a substrate to a diverse range of benzylisoquinoline alkaloids.

In some examples, the BIA precursors may be 4-HPAA and dopamine. In some examples, the BIA precursors may be 3,4-DHPA and dopamine. In some cases, a condensation reaction between two BIA precursors occurs between an amine of a first substrate and an aldehyde of a second substrate to generate an iminium ion followed by carbon-carbon bond formation between the C-6 of the first substrate and C-1 of the second substrate as provided in FIG. 1 and as represented generally in Scheme 4. As provided in Scheme 4, R₁, R₂, R₃, and R₄ may be H or OH.

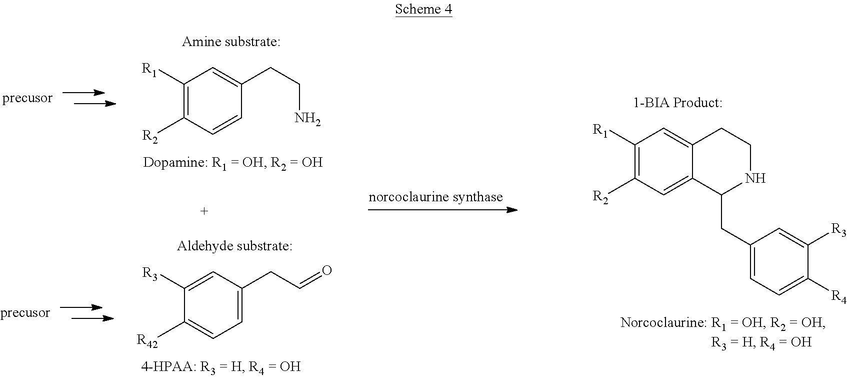

In some examples, the condensation of the BIA precursors to the 1-benzylisoquinoline alkaloid product may occur spontaneously. In some examples, the condensation reaction is promoted by conditions such as pH or solvent. In other examples, the 1-benzylisoquinoline alkaloid-generating Pictet-Spengler cyclization reaction is promoted by contact with a protein or enzyme with norcoclaurine synthase activity, or a norcoclaurine synthase. In some examples, this enzyme is a Bet v 1-fold protein. In some examples, this enzyme is an engineered norcoclaurine synthase. In some examples, this enzyme is an engineered norcoclaurine synthase with a truncation of its N-terminal sequence. In some examples, the enzyme encoding norcoclaurine synthase activity may catalyze the condensation reaction within a host cell, such as an engineered host, as described herein.

In some examples, the norcoclaurine synthase enzyme may be a Bet v 1 fold protein from plants in the Ranunculales order that biosynthesize thebaine, for example P. somniferum. In some examples, the norcoclaurine synthase enzyme may be a Bet v 1 fold protein from plants in the Ranunculales order that biosynthesize benzylisoquinoline alkaloids, for example C. japonica or E. californica. In some examples, the Bet v 1 protein includes the following domains in order from the N-terminus to C-terminus: a β-strand, one or two α-helices, six β-strands, and one or two α-helices. In some examples, a truncation is performed at the N-terminus of the enzyme to remove all or part of the first domain. In some examples, the enzyme may have one or more activity-increasing components as discussed herein and as described in Examples 14, 15, and 16. The protein is organized such that it has a Bet v 1 fold and an active site that accepts large, bulky, hydrophobic molecules, such as 1-benzylisoquinoline alkaloids. This protein may be any plant Bet v 1 protein.

In some examples, the enzyme with norcoclaurine synthase activity may be from mammals or any other vertebrate or invertebrate that biosynthesizes endogenous morphine.

In some examples, the norcoclaurine synthase may be combined with additional accessory proteins that may function to convert any BIA precursors into 1-benzylisoquinoline alkaloids. In some examples, these enzymes catalyze the reactions within a host cell, such as an engineered host, as described herein.

Examples of amino acid sequences for norcoclaurine synthases are set forth in Table 5. An amino acid sequence for a norcoclaurine synthase that is utilized in converting BIA precursors to 1-benzylisoquinoline alkaloid may be 75% or more identical to a given amino acid sequence as listed in Tables 6, 7, and 8. For example, an amino acid sequence for such a norcoclaurine synthase may comprise an amino acid sequence that is at least 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the nucleic acid sequence encoding the amino acid sequence is altered such as to optimize codon usage for the host organism, for example.

Amino acid residues of homologous norcoclaurine synthases may be referenced according to the numbering scheme of SEQ ID NO. 70, and this numbering system is used throughout the disclosure to refer to specific amino acid residues of norcoclaurine synthases which are homologous to SEQ ID NO. 70. Norcoclaurine synthases homologous to SEQ ID NO. 70 may have at least about 70%, 75%, 80%, 85%, 90%, 95%, 97%, 98%, or 99% sequence identity to SEQ ID NO. 70. In some cases, an amino acid referred to as position 50 in a homologous norcoclaurine synthase may not be the 50^{th} amino acid in the homologous norcoclaurine synthase, but would be the amino acid which corresponds to the amino acid at position 50 in SEQ ID NO. 70 in a protein alignment of the homologous norcoclaurine synthase with SEQ ID NO. 70. In some cases, homologous enzymes may be aligned with SEQ ID NO. 70 either according to primary sequence, secondary structure, or tertiary structure.

An engineered host cell may be provided that produces an engineered norcoclaurine synthase that converts BIA precursors to 1-benzylisoquinoline alkaloid, wherein the engineered norcoclaurine synthase comprises an amino acid sequence selected from the group consisting of: SEQ ID NOs: 69, 70, 71, 72, 73, 74, 75, 76, 77, 78, 79, 80, 81, and 82, and having one or more activity-enhancing modifications as described in Tables 6, 7, and 8. The engineered norcoclaurine synthase that is produced within the engineered host cell may be recovered and purified so as to form a biocatalyst. In some cases, the engineered norcoclaurine synthase may have a N-terminal truncation. These engineered norcoclaurine synthase enzymes may also be used to catalyze the conversion of BIA precursors to 1-benzylisoquinoline alkaloids. Additionally, the use of an engineered norcoclaurine synthase may be used to increase the production of benzylisoquinoline alkaloid products within a cell when compared to the production of benzylisoquinoline alkaloid products within a cell utilizing a parent norcoclaurine synthase.

In additional cases, the one or more enzymes that are recovered from the engineered host cell that produces the norcoclaurine synthase may be used in a process for converting BIA precursors to a 1-benzylisoquinoline alkaloid. The process may include contacting the BIA precursors with the recovered enzymes in an amount sufficient to convert said BIA precursors to 1-benzylisoquinoline alkaloid. In examples, the BIA precursors may be contacted with a sufficient amount of the one or more enzymes such that at least 5% of said BIA precursors is converted to 1-benzylisoquinoline alkaloid. In further examples, the BIA precursors may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said BIA precursors are converted to 1-benzylisoquinoline alkaloid.

In some examples, one or more enzymes converting BIA precursors to a 1-benzylisoquinoline alkaloid are localized to cellular compartments. In some examples, Bet v 1 may be modified such that it encodes targeting sequences that localize it to the endoplasmic reticulum membrane of the engineered host cell. In particular, in certain instances, the host cell may be engineered to increase production of norcoclaurine or norlaudanosoline or products for which norcoclaurine or norlaudanosline is a precursor from BIA precursors by localizing Bet v 1 to organelles in the yeast cell. Bet v 1 and/or DODC may be localized to the yeast endoplasmic reticulin in order to decrease the spatial distance between Bet v 1 and/or DODC. By increased production is meant both the production of some amount of the compound of interest where the control has no production of the compound of interest, as well as an increase of 10% or more, such as 50% or more, including 2-fold or more, e.g., 5-fold or more, such as 10-fold or more in situations where the control has some production of the compound of interest.

In other examples, DODC and Bet v 1 may be co-localized to a single protein fusion. In some examples, the fusion is created between DODC and Bet v 1 by one of several methods, including, direct fusion, co-localization to a yeast organelle, or by enzyme co-localization tools such as leucine zippers, protein scaffolds that utilize adapter domains, or RNA scaffolds that utilize aptamers. Co-localizing the norcoclaurine synthase enzyme may facilitate substrate channeling between the active sites of the enzymes and limit the diffusion of unstable intermediates such as 4-HPAA.

In some examples, an enzyme with DODC activity is fused to a peptide with a Bet v 1 fold. In some examples, the DODC enzyme and the Bet v 1 fold protein may be fused in any order from N-terminus to C-terminus, C-terminus to N-terminus, N-terminus to N-terminus, or C-terminus to C-terminus. In some examples, the two protein sequences may be fused directly or fused through a peptide linker region.

In some examples, an enzyme with DODC activity is fused to a peptide with a Bet v 1 fold by circular permutation. In some cases, the N- and C-termini of DODC are fused and the Bet v 1 sequence is then inserted randomly within this sequence. In some cases, the resulting fusion protein library is screened for 1-benzylisoquinoline alkaloid production.

The one or more enzymes that may be used to convert BIA precursors to a 1-benzylisoquinoline alkaloid may contact the BIA precursors in vitro. Additionally, or alternatively, the one or more enzymes that may be used to convert BIA precursors to a 1-benzylisoquinoline alkaloid may contact the BIA precursors in vivo. Additionally, the one or more enzymes that may be used to convert BIA precursors to a 1-benzylisoquinoline alkaloid may be provided to a cell having the BIA precursors within, or may be produced within an engineered host cell.

In some examples, the methods provide for engineered host cells that produce an alkaloid product, wherein the condensation of BIA precursors to a 1-benzylisoquinoline alkaloid product may comprise a key step in the production of an alkaloid product. In some examples, the alkaloid produced is a 1-benzylisoquinoline alkaloid. In still other embodiments, the alkaloid produced is derived from a 1-benzylisoquinoline alkaloid, including, for example, 4-ring promorphinan and 5-ring morphinan alkaloids. In another embodiment, a BIA precursor is an intermediate toward the product of the engineered host cell. In still other embodiments, the alkaloid product is selected from the group consisting of 1-benzylisoquinoline, promorphinan, morphinan, protoberberine, protopine, benzophenanthridine, secoberberine, phthalideisoquinoline, aporphine, bisbenzylisoquinoline, nal-opioid, or nor-opioid akaloids.

In some examples, the BIA precursor substrates are selected from the group consisting of 4-HPAA, 3,4-DHPA, and dopamine.

In some examples, the first BIA precursor substrate, or amine substrate, is a compound of Formula IX:
or a salt thereof, wherein

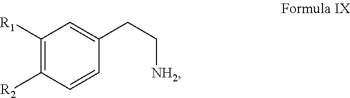

R₁ and R₂ are independently selected from hydrogen and hydroxy.

In some other examples, R₁ and R₂ are hydroxy, and the first BIA precursor substrate is dopamine.

In some examples, the second BIA precursor substrate, or aldehyde substrate, is a compound of Formula X:

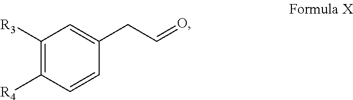

or a salt thereof, wherein:
R₃ and R₄ are independently selected from hydrogen and hydroxy.

In some examples, R₃ is a hydrogen and R₄ is a hydroxy, and the second BIA precursor is 4-HPAA.

In other examples, R₃ and R₄ are hydroxy, and the second BIA precursor is 3,4-DHPAA.

In some examples, the methods provide for engineered host cells that produce alkaloid products from BIA precursors. In some cases, the condensation of 4-HPAA and dopamine to norcoclaurine may comprise a key step in the production of diverse alkaloid products from a precursor. In some cases, the condensation of 3,4-DHPA and dopamine to norlaudanosoline may comprise a key step in the production of diverse alkaloid products from a precursor. In some examples, the precursor is L-tyrosine or a sugar (e.g., glucose). The diverse alkaloid products can include, without limitation, 1-benzylisoquinoline, promorphinan, morphinan, protoberberine, protopine, benzophenanthridine, secoberberine, phthalideisoquinoline, aporphine, bisbenzylisoquinoline, nal-opioid, and nor-opioid akaloids.

Any suitable carbon source may be used as a precursor toward a 1-benzylisoquinoline alkaloid. Suitable precursors can include, without limitation, monosaccharides (e.g., glucose, fructose, galactose, xylose), oligosaccharides (e.g., lactose, sucrose, raffinose), polysaccharides (e.g., starch, cellulose), or a combination thereof. In some examples, unpurified mixtures from renewable feedstocks can be used (e.g., cornsteep liquor, sugar beet molasses, barley malt, biomass hydrolysate). In still other embodiments, the carbon precursor can be a one-carbon compound (e.g., methanol, carbon dioxide) or a two-carbon compound (e.g., ethanol). In yet other embodiments, other carbon-containing compounds can be utilized, for example, methylamine, glucosamine, and amino acids (e.g., L-tyrosine). In some examples, a BIA precursor substrate may be added directly to an engineered host cell of the disclosure, including, for example, 4-HPAA, 3,4-DHPA, and/or dopamine.

In some examples, a benzylisoquinoline alkaloid product, or a derivative thereof, is recovered. In some examples, the benzylisoquinoline alkaloid product is recovered from a cell culture. In some examples, the benzylisoquinoline alkaloid product is a 1-benzylisoquinoline, promorphinan, morphinan, protoberberine, protopine, benzophenanthridine, secoberberine, phthalideisoquinoline, aporphine, bisbenzylisoquinoline, nal-opioid, or nor-opioid akaloids. O-Demethylation Modifications

Some methods, processes, and systems provided herein describe the conversion of a first benzylisoquinoline alkaloid to a second benzylisoquinoline alkaloid by the removal of an O-linked methyl group. Some of these methods, processes, and systems may comprise an engineered host cell. In some examples, the conversion of a first benzylisoquinoline alkaloid to a second benzylisoquinoline alkaloid is a key step in the conversion of a substrate to a nor-opioids or nal-opioids. In some examples, the conversion of a first alkaloid to a second alkaloid comprises a demethylase reaction.

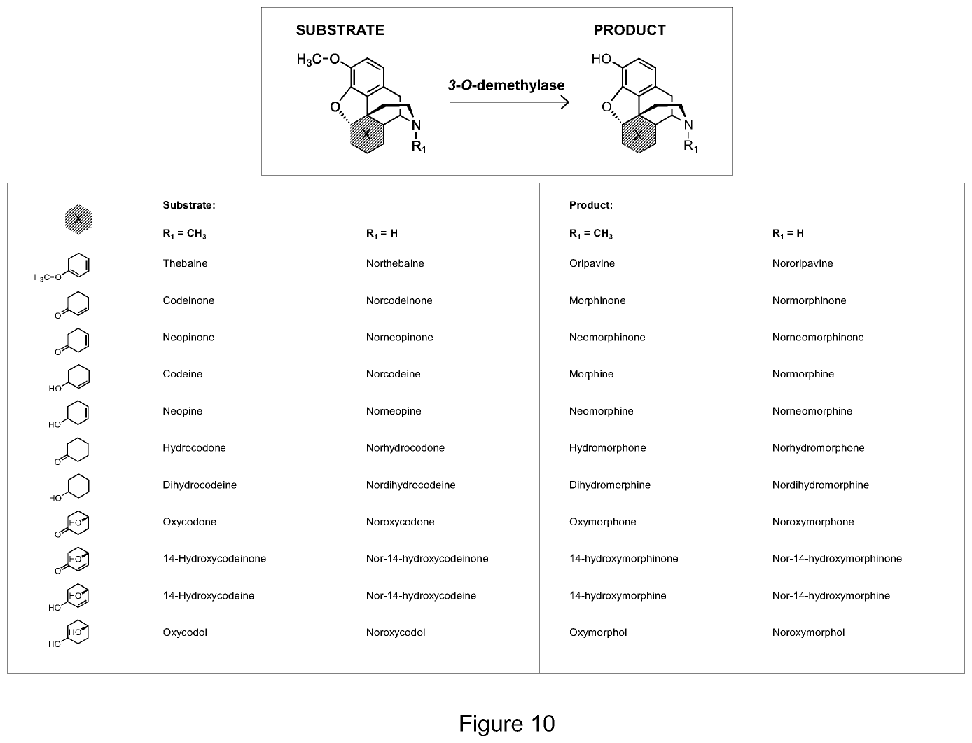

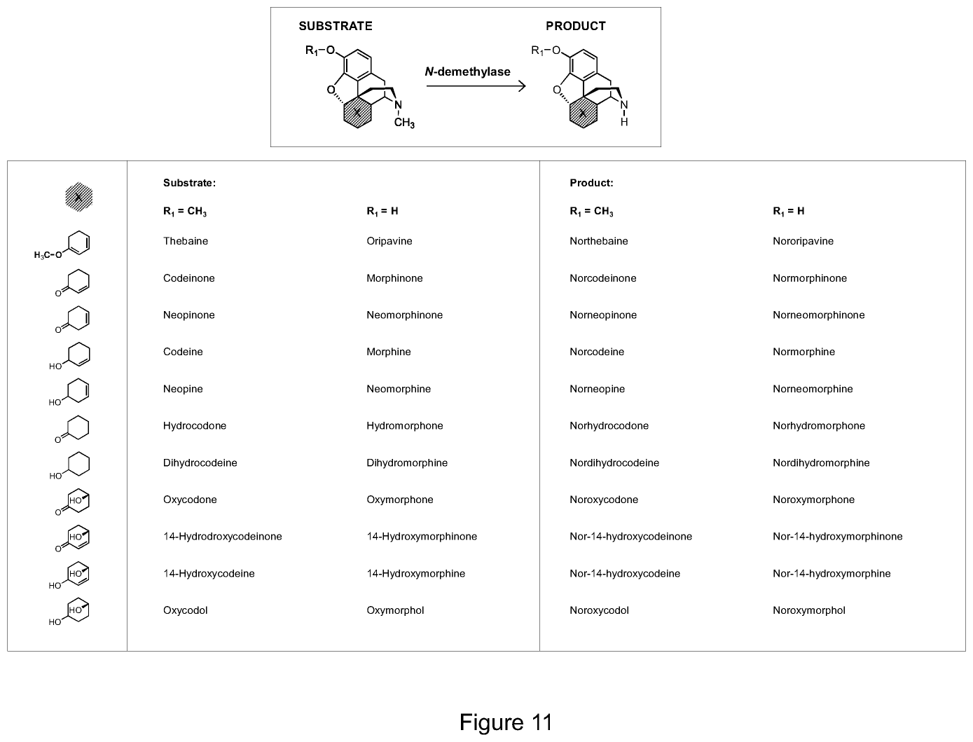

FIG. 10 illustrates an enzyme having opioid 3-O-demethylase (ODM) activity, in accordance with some embodiments of the invention. Specifically, the enzyme may act on morphinan alkaloid structures to remove the methyl group from the oxygen bound to carbon 3.

Examples of amino acid sequences of ODM enzymes are set forth in Table 12. An amino acid sequence for an ODM that is utilized in converting a first alkaloid to a second alkaloid may be 50% or more identical to a given amino acid sequence as listed in Table 12. For example, an amino acid sequence for such an epimerase may comprise an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the DNA sequence is altered such as to optimize codon usage for the host organism, for example.

An engineered host cell may be provided that produces an ODM that converts a first alkaloid to a second alkaloid, wherein the ODM comprises a given amino acid sequence as listed in Table 12. An engineered host cell may be provided that produces one or more ODM enzymes. The ODM that is produced within the engineered host cell may be recovered and purified so as to form a biocatalyst. The process may include contacting the first alkaloid with an ODM in an amount sufficient to convert said first alkaloid to a second alkaloid. In some examples, the first alkaloid may be contacted with a sufficient amount of the one or more enzymes such that at least 5% of said first alkaloid is converted to a second alkaloid. In further examples, the first alkaloid may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said first alkaloid is converted to a second alkaloid.

The one or more enzymes that may be used to convert a first alkaloid to a second alkaloid may contact the first alkaloid in vitro. Additionally, or alternatively, the one or more enzymes that may be used to convert a first alkaloid to a second alkaloid may contact the first alkaloid in vivo. In some examples, the one or more enzymes that may be used to convert a first alkaloid to a second alkaloid may be provided to a cell having the first alkaloid within. In some examples, the one or more enzymes that may be used to convert a first alkaloid to a second alkaloid may be produced within an engineered host cell.

In some examples, the methods provide for engineered host cells that produce an alkaloid product, wherein the O-demethylation of a substrate to a product may comprise a key step in the production of an alkaloid product. In some examples, the alkaloid produced is a nor-opioid or a nal-opioid. In still other embodiments, the alkaloid produced is derived from a nor-opioid or a nal-opioid. In another embodiment, a first alkaloid is an intermediate toward the product of the engineered host cell. In still other embodiments, the alkaloid product is selected from the group consisting of morphine, oxymorphine, oripavine, hydromorphone, dihydromorphine, 14-hydroxymorphine, morphinone, and 14-hydroxymorphinone.

In some examples, the substrate alkaloid is an opioid selected from the group consisting of codeine, oxycodone, thebaine, hydrocodone, dihydrocodeine, 14-hydroxycodeine, codeinone, and 14-hydroxycodeinone.

### Heterologous Coding Sequences

In some instances, the engineered host cells harbor one or more heterologous coding sequences (such as two or more, three or more, four or more, five or more) which encode activity(ies) that enable the engineered host cells to produce desired enzymes of interest and/or BIAs of interest, e.g., as described herein. As used herein, the term “heterologous coding sequence” is used to indicate any polynucleotide that codes for, or ultimately codes for, a peptide or protein or its equivalent amino acid sequence, e.g., an enzyme, that is not normally present in the host organism and may be expressed in the host cell under proper conditions. As such, “heterologous coding sequences” includes multiple copies of coding sequences that are normally present in the host cell, such that the cell is expressing additional copies of a coding sequence that are not normally present in the cells. The heterologous coding sequences may be RNA or any type thereof, e.g., mRNA, DNA or any type thereof, e.g., cDNA, or a hybrid of RNA/DNA. Coding sequences of interest include, but are not limited to, full-length transcription units that include such features as the coding sequence, introns, promoter regions, 3′-UTRs, and enhancer regions.

In some examples, the engineered host cells may comprise a plurality of heterologous coding sequences each encoding an enzyme, such as an enzyme listed in Tables 11, 17 (e.g., P450), and 18 (e.g., CPR). In some examples, the plurality of enzymes encoded by the plurality of heterologous coding sequences may be distinct from each other. In some examples, some of the plurality of enzymes encoded by the plurality of heterologous coding sequences may be distinct from each other and some of the plurality of enzymes encoded by the plurality of heterologous coding sequences may be duplicate copies.

In some examples, the heterologous coding sequences may be operably connected. Heterologous coding sequences that are operably connected may be within the same pathway of producing a particular benzylisoquinoline alkaloid product and/or epimerase product. In some examples, the operably connected heterologous coding sequences may be directly sequential along the pathway of producing a particular benzylisoquinoline alkaloid product and/or epimerase product. In some examples, the operably connected heterologous coding sequences may have one or more native enzymes between one or more of the enzymes encoded by the plurality of heterologous coding sequences. In some examples, the heterologous coding sequences may have one or more heterologous enzymes between one or more of the enzymes encoded by the plurality of heterologous coding sequences. In some examples, the heterologous coding sequences may have one or more non-native enzymes between one or more of the enzymes encoded by the plurality of heterologous coding sequences.

The engineered host cells may also be modified to possess one or more genetic alterations to accommodate the heterologous coding sequences. Alterations of the native host genome include, but are not limited to, modifying the genome to reduce or ablate expression of a specific protein that may interfere with the desired pathway. The presence of such native proteins may rapidly convert one of the intermediates or final products of the pathway into a metabolite or other compound that is not usable in the desired pathway. Thus, if the activity of the native enzyme were reduced or altogether absent, the produced intermediates would be more readily available for incorporation into the desired product.

Heterologous coding sequences include but are not limited to sequences that encode enzymes, either wild-type or equivalent sequences, that are normally responsible for the production of BIAs of interest in plants. In some cases, the enzymes for which the heterologous sequences code may be any of the enzymes in the 1-benzylisoquinoline alkaloid pathway, and may be from any convenient source. The choice and number of enzymes encoded by the heterologous coding sequences for the particular synthetic pathway may be selected based upon the desired product. In certain embodiments, the host cells of the disclosure may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more heterologous coding sequences, such as 1, 2, 3, 4, 5, 6, 7, 8, 9, 10, 11, 12, 13, 14, or 15 heterologous coding sequences.

As used herein, the term “heterologous coding sequences” also includes the coding portion of the peptide or enzyme, i.e., the cDNA or mRNA sequence, of the peptide or enzyme, as well as the coding portion of the full-length transcriptional unit, i.e., the gene including introns and exons, as well as “codon optimized” sequences, truncated sequences or other forms of altered sequences that code for the enzyme or code for its equivalent amino acid sequence, provided that the equivalent amino acid sequence produces a functional protein. Such equivalent amino acid sequences may have a deletion of one or more amino acids, with the deletion being N-terminal, C-terminal, or internal. Truncated forms are envisioned as long as they have the catalytic capability indicated herein. Fusions of two or more enzymes are also envisioned to facilitate the transfer of metabolites in the pathway, provided that catalytic activities are maintained.

Operable fragments, mutants, or truncated forms may be identified by modeling and/or screening. In some cases, this is achieved by deletion of, for example, N-terminal, C-terminal, or internal regions of the protein in a step-wise fashion, followed by analysis of the resulting derivative with regard to its activity for the desired reaction compared to the original sequence. If the derivative in question operates in this capacity, it is considered to constitute an equivalent derivative of the enzyme proper.

In some examples, some heterologous proteins may show occurrences where they are incorrectly processed when expressed in a recombinant host. For example, plant proteins such as cytochrome P450 enzymes expressed in microbial production hosts may have occurrences of incorrect processing. In particular, salutaridine synthase may undergo N-linked glycosylation when heterologously expressed in yeast. This N-linked glycosylation may not be observed in plants, which may be indicative of incorrect N-terminal sorting of the nascent SalSyn transcript so as to reduce the activity of the enzyme in the heterologous microbial host. In such examples, protein engineering directed at correcting N-terminal sorting of the nascent transcript so as to remove the N-linked glycosylation pattern may result in improved activity of the salutaridine synthase enzyme in the recombinant production host.

Aspects of the disclosure also relate to heterologous coding sequences that code for amino acid sequences that are equivalent to the native amino acid sequences for the various enzymes. An amino acid sequence that is “equivalent” is defined as an amino acid sequence that is not identical to the specific amino acid sequence, but rather contains at least some amino acid changes (deletions, substitutions, inversions, insertions, etc.) that do not essentially affect the biological activity of the protein as compared to a similar activity of the specific amino acid sequence, when used for a desired purpose. The biological activity refers to, in the example of an epimerase, its catalytic activity. Equivalent sequences are also meant to include those which have been engineered and/or evolved to have properties different from the original amino acid sequence. Mutable properties of interest include catalytic activity, substrate specificity, selectivity, stability, solubility, localization, etc.

In some instances, the expression of each type of enzyme is increased through additional gene copies (i.e., multiple copies), which increases intermediate accumulation and/or BIA of interest production. Some embodiments of the disclosure include increased BIA of interest production in a host cell through simultaneous expression of multiple species variants of a single or multiple enzymes. In some cases, additional gene copies of a single or multiple enzymes are included in the host cell. Any convenient methods may be utilized including multiple copies of a heterologous coding sequence for an enzyme in the host cell.

In some examples, the engineered host cell includes multiple copies of a heterologous coding sequence for an enzyme, such as 2 or more, 3 or more, 4 or more, 5 or more, or even 10 or more copies. In certain embodiments, the engineered host cell includes multiple copies of heterologous coding sequences for one or more enzymes, such as multiple copies of two or more, three or more, four or more, etc. In some cases, the multiple copies of the heterologous coding sequence for an enzyme are derived from two or more different source organisms as compared to the host cell. For example, the engineered host cell may include multiple copies of one heterologous coding sequence, where each of the copies is derived from a different source organism. As such, each copy may include some variations in explicit sequences based on inter-species differences of the enzyme of interest that is encoded by the heterologous coding sequence.

In certain embodiments, the engineered host cell includes multiple copies of heterologous coding sequences for one or more enzymes, such as multiple copies of two or more, three or more, four or more, etc. In some cases, the multiple copies of the heterologous coding sequence for an enzyme are derived from two or more different source organisms as compared to the host cell. For example, the engineered host cell may include multiple copies of one heterologous coding sequence, where each of the copies is derived from a different source organism. As such, each copy may include some variations in explicit sequences based on inter-species differences of the enzyme of interest that is encoded by the heterologous coding sequence.

The engineered host cell medium may be sampled and monitored for the production of BIAs of interest. The BIAs of interest may be observed and measured using any convenient methods. Methods of interest include, but are not limited to, LC-MS methods (e.g., as described herein) where a sample of interest is analyzed by comparison with a known amount of a standard compound. Additionally, there are other ways that BIAs of interest may be observed and/or measured. Examples of alternative ways of observing and/or measuring BIAs include GC-MS, UV-vis spectroscopy, NMR, LC-NMR, LC-UV, TLC, capillary electrophoresis, among others. Identity may be confirmed, e.g., by m/z and MS/MS fragmentation patterns, MRM transitions, and quantitation or measurement of the compound may be achieved via LC trace peaks of know retention time and/or EIC MS peak analysis by reference to corresponding LC-MS analysis of a known amount of a standard of the compound. In some cases, identity may be confirmed via multiple reaction monitoring using mass spectrometry.

Additionally, a culture of the engineered host cell may be sampled and monitored for the production of enzymes of interest, such as a neopinone isomerase enzyme. The enzymes of interest may be observed and measured using any convenient methods. Methods of interest include enzyme activity assays, polyacrylamide gel electrophoresis, carbon monoxide spectroscopy, and western blot analysis. Formaldehyde Toxicity Alleviating Modifications

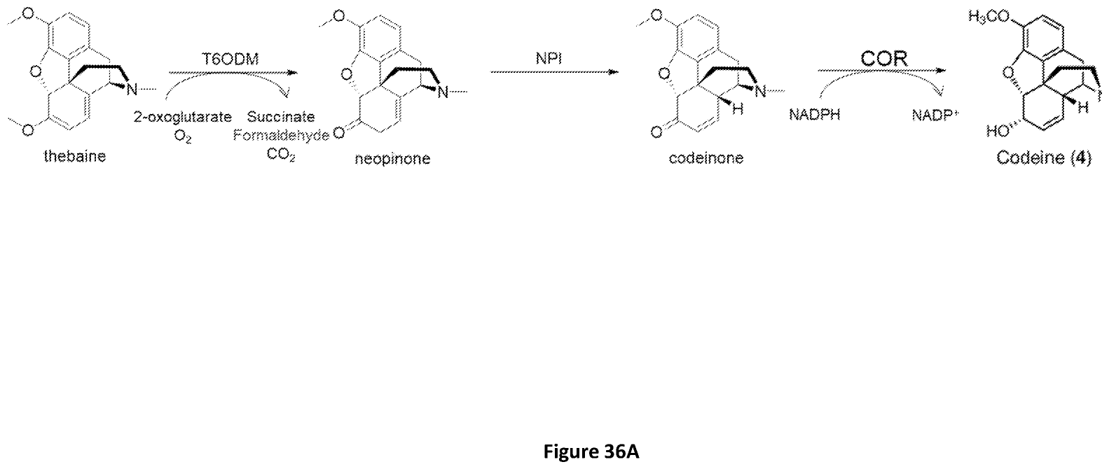

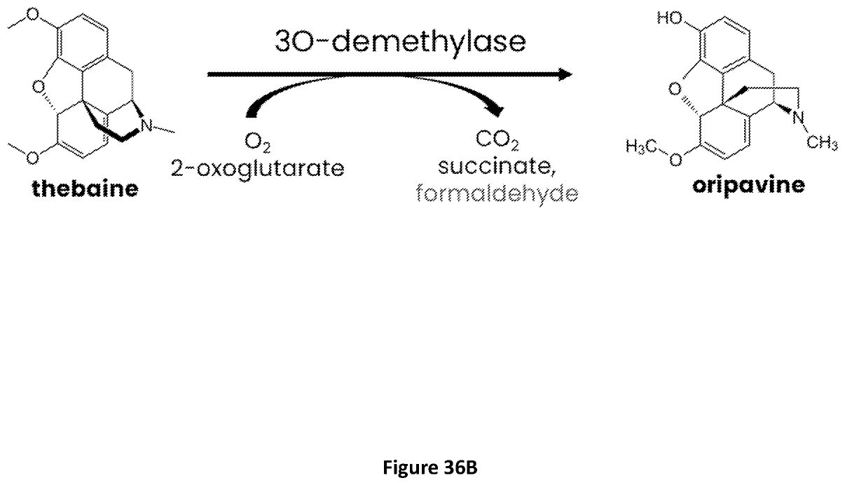

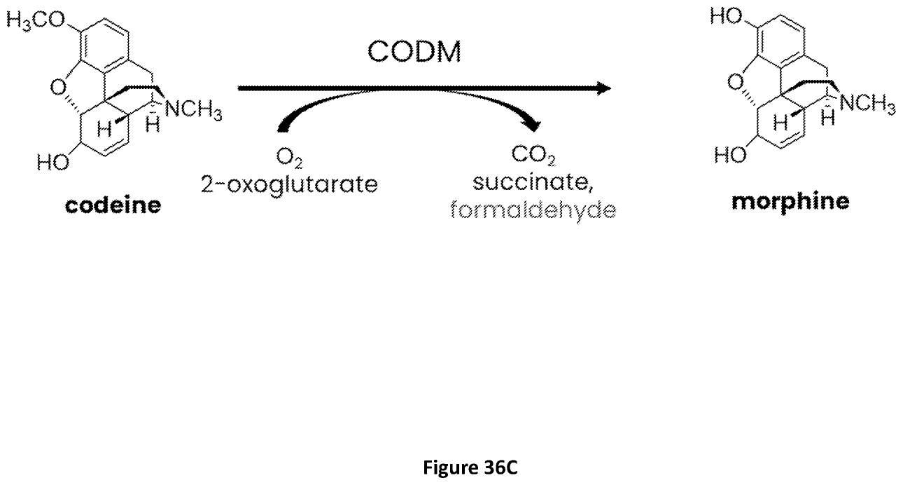

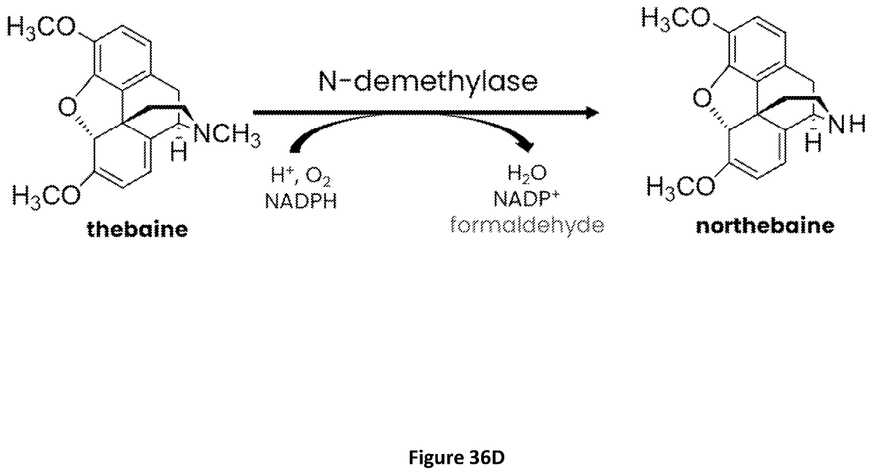

The host cells may include one or more modifications (such as two or more, three or more, four or more, five or more, or even more modifications) of one or more biosynthetic enzyme genes of the cell that are directed to alleviating formaldehyde toxicity. In some examples, the one or more biosynthetic enzyme genes are native to the cell. In some examples, the one or more biosynthetic enzyme genes are non-native (e.g., heterologous) to the cell. Any convenient biosynthetic enzyme genes of the cell may be targeted for modification to alleviate accumulation of formaldehyde. As used herein, the term “formaldehyde toxicity alleviating modification” refers to a modification that reduces the accumulation formaldehyde that may be produced as a byproduct of a biosynthetic process in an engineered host cell. Formaldehyde toxicity is a process that occurs in a cell when formaldehyde builds up in a cell. For example, biosynthetic processes involving oxidation of a methyl group for demethylation produces the undesired byproduct formaldehyde, which is toxic to yeast. For example, to produce noroxymorphone, a yeast strain must also produce intermediates in the noroxymorphone pathway, particularly benzylisoquinoline alkaloids (BIAs) such as codeinone, codeine, hydrocodone, morphinone, and other morphanin intermediates. One key step in pathway to produce noroxymorphone from these morphanin intermediates is oxidation of an intermediate methylated at position 6 of a morphanin intermediate, such as thebaine, oripavine, northebaine, or nororipavine or others. This demethylation is known to be catalyzed by the 2-oxoglutarate and Fe(II)-dependent dioxygenase (20DD) enzyme, thebaine 6-O-demethylase (T6ODM), or any other oxidase with similar activity. Depending on the substrate for the reaction, products include neopinone, neomorphinone, norneopinone (N-demethylate neopinone), norneomorphinone (N-demethylated neomorphinone) or others. Regardless of the oxidative enzyme used, the substrate, or product, oxidation of a methyl group for demethylation produces the undesired byproduct formaldehydeFor example, FIGS. 36A-36D depict exemplary metabolic pathways showing oxidation of morphinans resulting in the production of formaldehyde as a biproduct. FIG. 36A depicts an exemplary metabolic pathway showing codeine biosynthesis using the conversion from thebaine to codeine, which involves demethylation of thebaine at position 6 (shown as catalyzed by T6ODM in FIG. 36A). As can be seen in FIG. 36A, formaldehyde is a necessary byproduct of this reaction. FIG. 36B depicts another exemplary metabolic pathway showing biosynthesis of a morphinan (here, oripavine) from thebaine. In this exemplary pathway, thebaine is oxidized by 3-O-demethylase to form oripavine, generating formaldehyde as a byproduct of the reaction. FIG. 36C depicts yet another exemplary metabolic pathway showing biosynthesis of a morphinan (here, morphine) from thebaine. In this exemplary pathway, thebaine is oxidized by CODM to form morphine, generating formaldehyde as a byproduct of the reaction. FIG. 36D depicts yet another exemplary metabolic pathway showing biosynthesis of a morphanane (here, northebaine) from thebaine. In this exemplary pathway, thebaine is oxidized by N-demethylase to form northebaine, generating formaldehyde as a byproduct of the reaction. Other demethylations of morphinans can be used without departing from the present disclosure (e.g., a methylation may occur at the 3-position or the nitrogen group and produce formaldehyde).

A modification that alleviates formaldehyde toxicity in an engineered host cell reduces the accumulation of formaldehyde in the engineered host cell relative to a control cell. In this way, the engineered host cell provides for a decreased level of formaldehyde and/or an increased level of the BIAs of interest. By increased level is meant a level that is 110% or more of that of the BIAs of interest in a control cell, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the BIAs of interest in the control cell. By decreased level is meant a level that is reduced by 10% or more, such as by 20% or more, 30% or more, 40% or more, 50% or more, 60% or more, 70% or more, 80% or more, 90% or more, 95% or more, 97% or more, 99% or more, about 10%, about 20%, about 20%, about 40%, about 50%, about 60%, about 70%, about 80%, about 90%, about 95%, about 97%, about 99%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 100% of that of the formaldehyde accumulated in a control cell.

A variety of formaldehyde toxicity alleviating modifications and biosynthetic enzymes in the engineered host cell that are directed to reducing the accumulation of formaldehyde may be targeted for modification. The engineered host cell may include one or more formaldehyde toxicity alleviating modifications in one or more biosynthetic enzyme genes. In some examples, the engineered host cell includes a modification that increases the expression of a formaldehyde dehydrogenase. In some examples, the formaldehyde dehydrogenase is the enzyme SFA1.

In some examples, formaldehyde toxicity alleviating modifications may result in undesirable downstream effects. For example, formaldehyde detoxification may deplete the glutathione pool in a host cell. For example, FIG. 36 depicts the major detoxification pathway in yeast, which utilizes the formaldehyde dehydrogenase enzyme SFA1. The first step is spontaneous conjugation of formaldehyde to glutathione (the thiol group of glutathione is specifically shown to demonstrate where the reaction occurs on the molecule). The second step is oxidation to S-formylglutathione by SFA1. Glutathione, which is necessary for formaldehyde detoxification via SFA1, can be depleted by catabolism to glutamate and cysteinylglycine which is catalyzed by DUG2/3. In this example, the undesirable downstream effect of depleting the glutathione pool may be alleviated by introducing a modification that is directed to maintain the glutathione pool. In some examples, the modification that is directed to maintain the glutathione pool reduces or knocks out the expression of DUG2 and/or DUG3 or any other suitable enzyme.

Any convenient numbers and types of modifications may be utilized to alleviate formaldehyde toxicity and/or an undesirable downstream effect. In certain embodiments, the engineered host cells of the present disclosure may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more modifications to alleviate formaldehyde toxicity and/or an undesirable downstream effect, such as 1, 2, 3, 4, 5, 6, 7, 8, 9, 10, 11, 12, 13, 14 or 15 modifications in one or more biosynthetic enzyme genes within the engineered host cell. 14-Hydroxylation Modifications

Some methods, processes, and systems provided herein include the conversion of first morphanin alkaloid to a second morphanin alkaloid by adding a hydroxyl group to a free hydrogen at the C14-position of the first morphanin alkaloid. An exemplary morphanin alkaloid with a free hydrogen at the C14-position is depicted below in Formula XI. Some of these methods, processes, and systems may comprise an engineered host cell. In some examples, the conversion of a first morphanin alkaloid to a second morphanin alkaloid is a key step in the conversion of a substrate to a nor-opioid. In some examples, the conversion of a first morphanin alkaloid to a second morphanin alkaloid comprises a hydroxylation reaction.

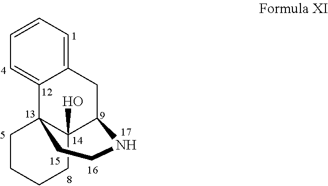

Any suitable enzyme providing 14-hydroxylase activity can be used to perform the hydroxylation of the free hydrogen at the 14 position of the first morphanin alkaloid. In some embodiments, the enzyme is a P450 enzyme.

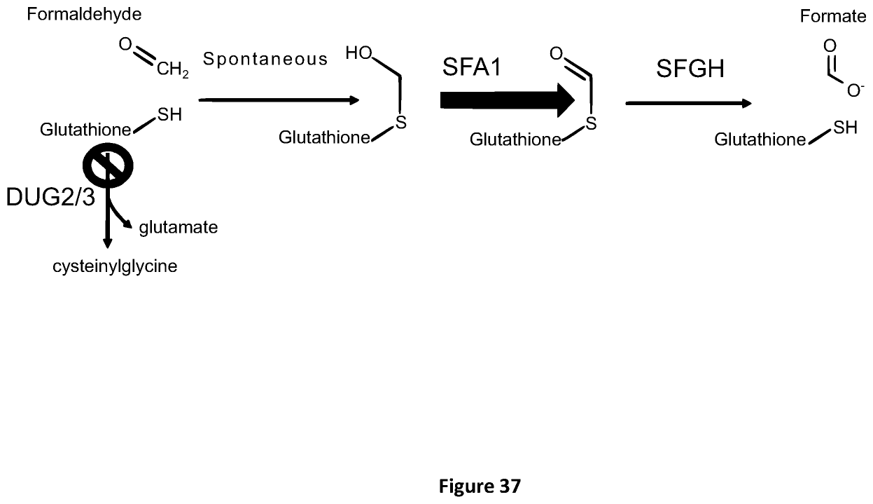

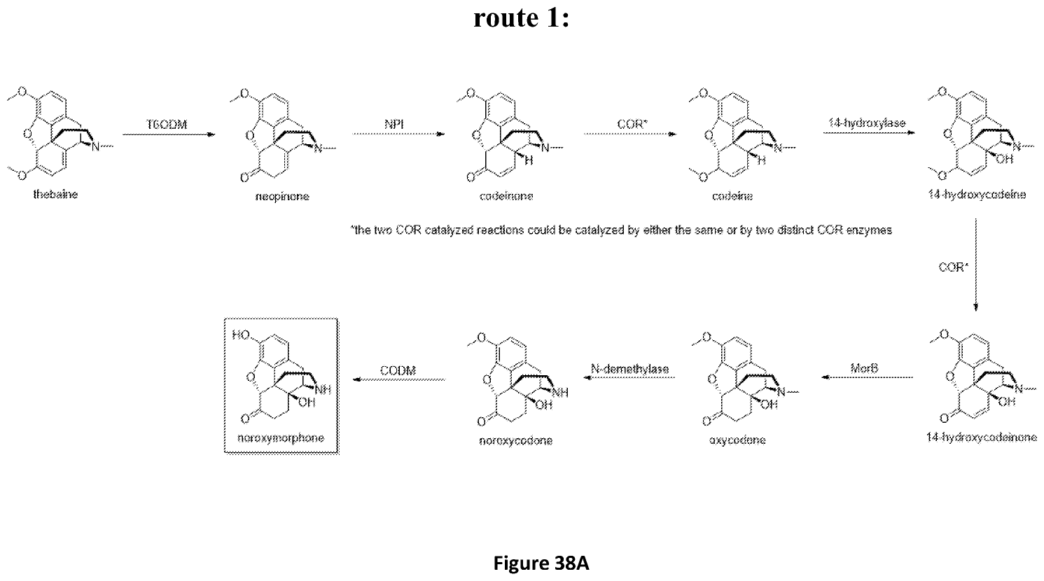

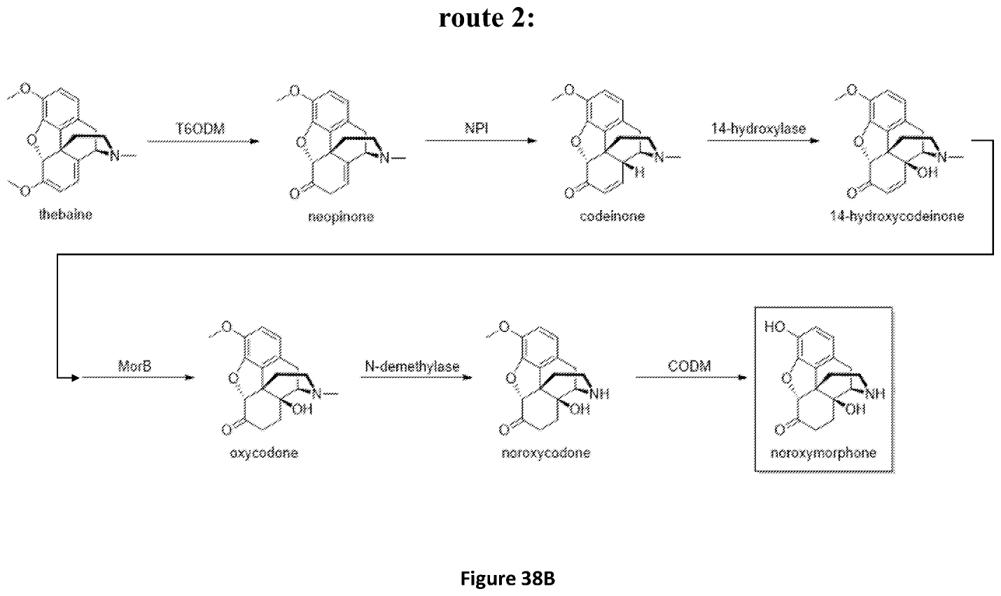

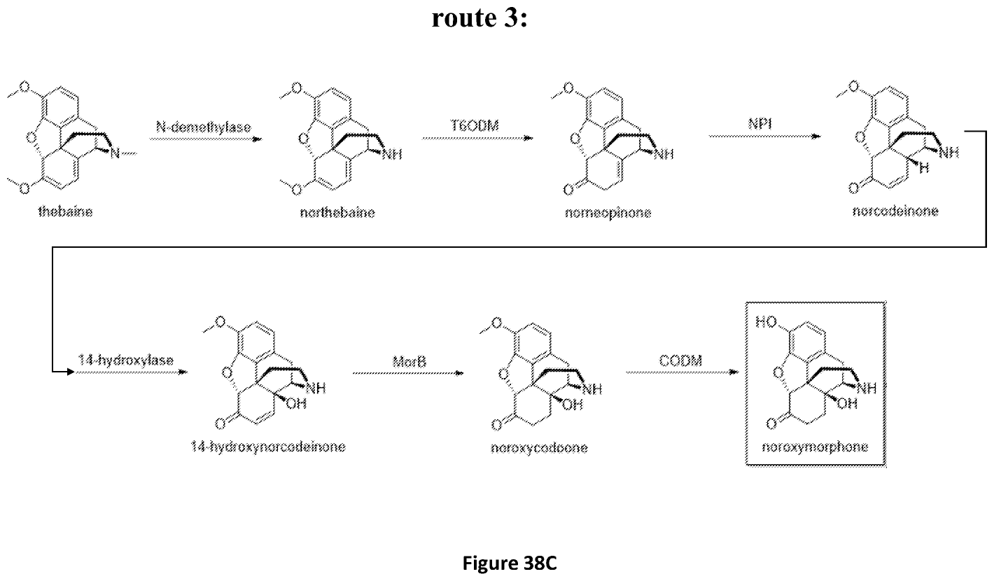

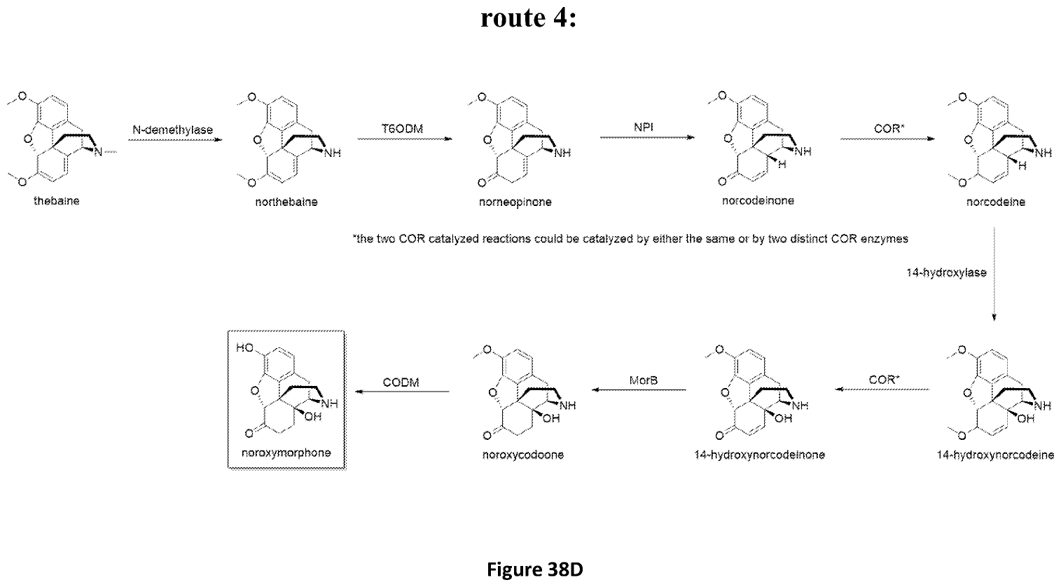

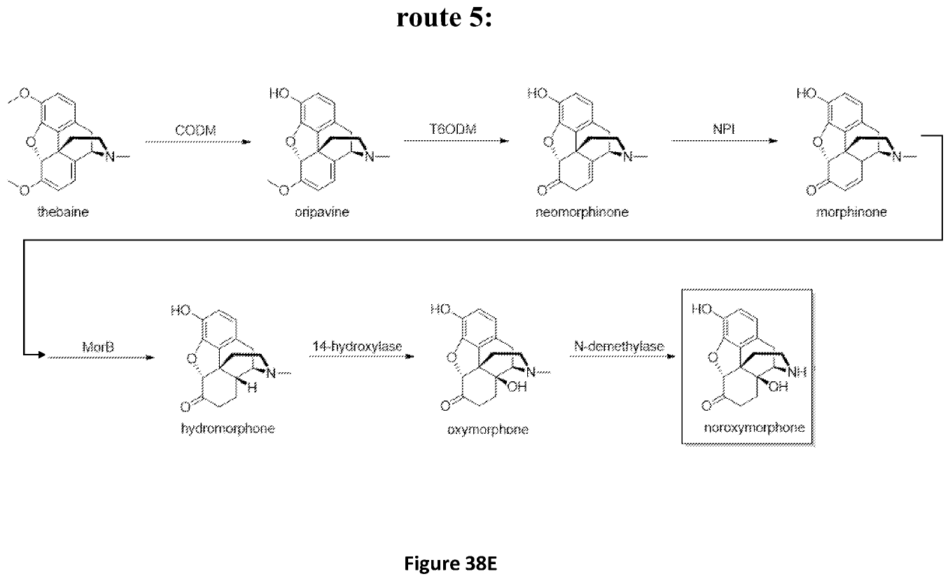

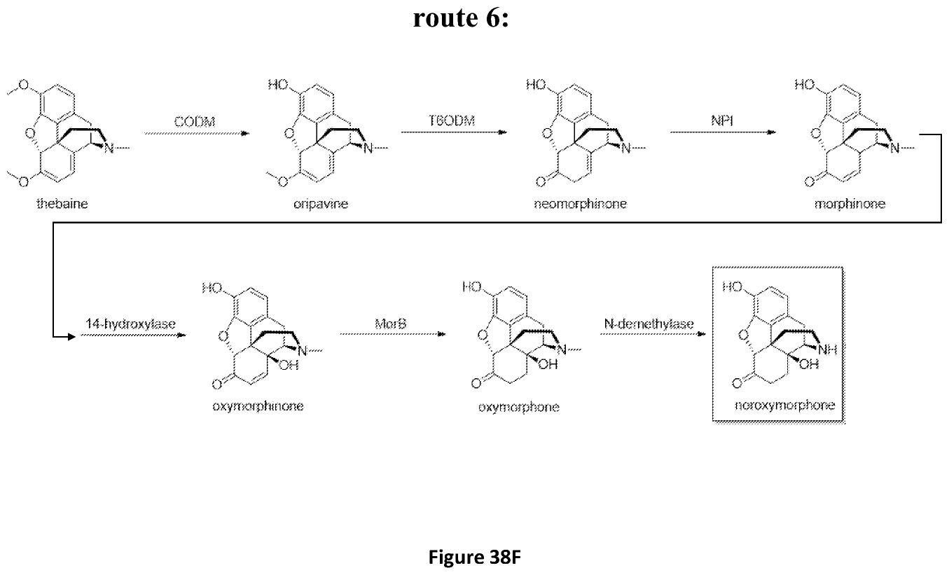

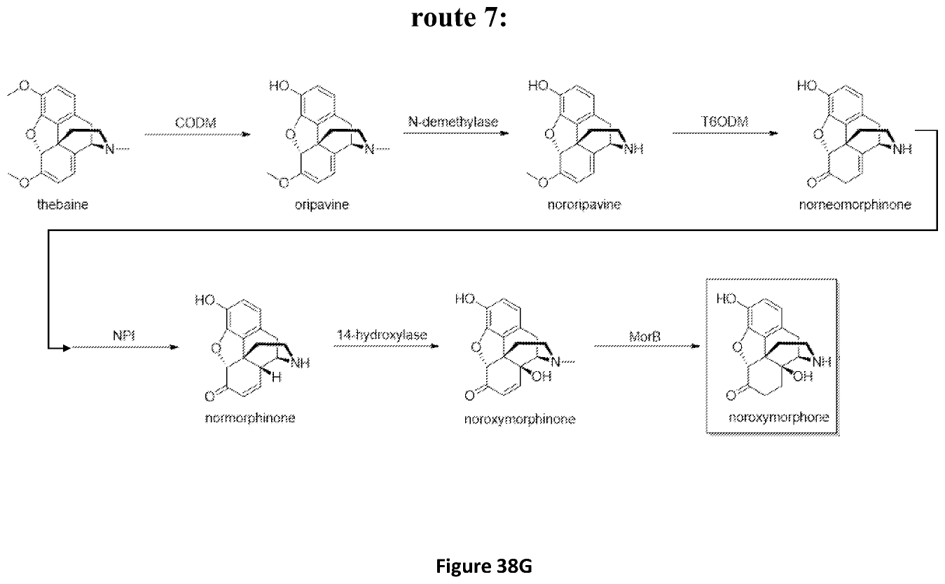

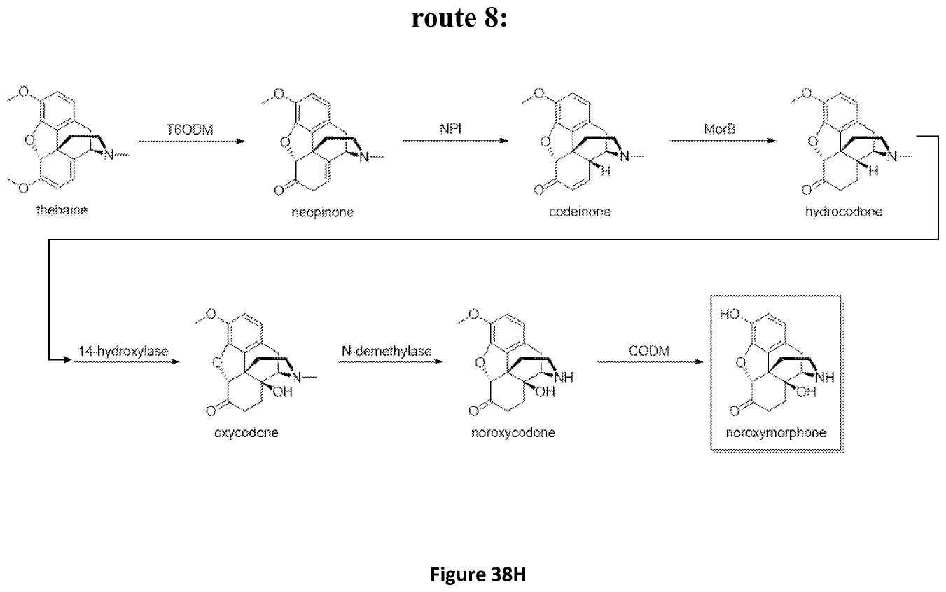

FIGS. 37A-H illustrate eight exemplary biosynthetic routes, each involving at least one enzyme having 14-hydroxylation activity, in accordance with some embodiments of the disclosure. Specifically, the enzyme may act on morphinan alkaloid structures to add a hydroxyl group to an available C—H group at the 14-position.

An enzyme that may add a hydroxyl group to an available C—H group at the 14-position of a morphanin alkaloid is an enzyme comprising 14-hydroxylase activity. In certain embodiments, the one or more proteins comprising 14-hydroxylase activity is heterologous to the engineered host cell. In some embodiments, the one or more proteins comprising 14-hydroxylase activity comprises a cytochrome p450 (P450) protein.

In some embodiments, the cytochrome P450 enzyme comprises one or more mutations relative to a wildtype sequence. In some embodiments, the wildtype sequence is 14HC_P450_5 (SEQ ID NO: 179). In some embodiments, the cytochrome P450 comprises one or mutations at positions 58, 59, 102, 181, 188, 189, 17, 280, 325, and/or 396. In some embodiments, the mutation is at position 17 and comprises an I or L substitution. In some embodiments, the mutation is at position 58 and comprises a K substitution. In some embodiments, the mutation is at position 59 and comprises a D substitution. In some embodiments, the mutation is at position 102 and comprises an L or M substitution. In some embodiments, the mutation is at position 181 and comprises a G, I, L, M, P, O, S, or V substitution. In some embodiments, the mutation is at position 188 and comprises an I substitution. In some embodiments, the mutation is at position 189 and comprises a V substitution. In some embodiments, the mutation is at position 208 and comprises an N substitution. In some embodiments, the mutation is at position 325 and comprises an I, M. or V substitution.

In some embodiments, the one or more mutations comprise E58K, A59D, F102L, D181E, L188I, and D189E mutations relative to SEQ ID NO: 179. In some embodiments, the cytochrome P450 enzyme comprises SEQ ID NO: 190.

In some embodiments, the one or more mutations comprise L188I and D189E mutations relative to SEQ ID NO: 179. In some embodiments, the cytochrome P450 enzyme comprises SEQ ID NO: 184.

In some embodiments, the one or mutations comprise A59D, L188I and D189E mutations relative to SEQ ID NO: 179. In some embodiments, the cytochrome P450 enzyme comprises SEQ ID NO: 186.

In some embodiments, the cytochrome P450 enzyme having an amino acid sequence having at least 90%, 95%, 96%, 97%, 98% or 99% sequence identity to SEQ ID NOs.: 190, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 178, 180, 182, 184, 186, 188, 192, 194, 196, 198, 200, 202, 204, 206, 208, 210, 212, 214, 216, 218, 220, 222, 224, 226, 228, 230, 232, 234, 236, 238, 240, 242, or 244.

In some embodiments, the heterologous enzyme comprises or consists of the amino acid sequence of SEQ ID NOs: 190, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 178, 180, 182, 184, 186, 188, 192, 194, 196, 198, 200, 202, 204, 206, 208, 210, 212, 214, 216, 218, 220, 222, 224, 226, 228, 230, 232, 234, 236, 238, 240, 242, or 244

In some embodiments, a P450 protein is expressed with a CPR enzyme. Examples of amino acid sequences of enzymes comprising 14-hydroxylase activity, and exemplary nucleic acid sequences encoding those amino acid sequences, are set forth in Table 17.

In some embodiments, the CPR is a fungal CPR. In some embodiments, the CPR is a plant CPR. In some embodiments, the CPR is an animal CPR.

In some embodiments, the P450 protein and the CPR enzyme are from the same genus. In some embodiments, both P450 protein and the CPR enzyme are from fungus.

In some embodiments, the CPR comprises an amino acid sequence having at least 90%, 95%, 96%, 97%, 98% or 99% sequence identity to SEQ ID NO: 168, 170, 172,174, 248, 250, 252, 254, 256, 258, 260, 262, 264, 266, 268, or 270.

In some embodiments, the CPR comprises or consists of SEQ ID NO: 168, 170, 172,174, 248, 250, 252, 254, 256, 258, 260, 262, 264, 266, 268, or 270.

In an example, an amino acid sequence for an enzyme comprising 14-hydroxylation activity that is utilized in converting a morphanin alkaloid to a 14-hydroxylated morphanin alkaloid may be 50% or more identical to a given amino acid sequence as listed in Table 17. For example, an amino acid sequence for such an enzyme comprising 14-hydroxylase activity may comprise an amino acid sequence that is at least 50% or more, 55% or more, 60% or more, 65% or more, 70% or more, 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the nucleic acid sequence encoding the amino acid sequence is altered such as to optimize codon usage for the host organism, for example.

An engineered host cell may be provided that expresses an enzyme providing 14-hydroxylation activity that converts a first alkaloid to a second alkaloid, wherein the enzyme comprising 14-hydroxylation activity comprises an amino acid sequence as provided in Table 17. An engineered host cell may be provided that expresses an enzyme providing 14-hydroxylation activity and a CPR enzyme, wherein the enzyme providing 14-hydroxylation activity comprises an amino acid sequence as provided in Table 17 and/or the CPR enzyme comprises an amino acid sequence as provided in Table 18. An engineered host cell may be provided that expresses one or more enzymes providing 14-hydroxylation activity. The enzyme providing 14-hydroxylation activity that is produced within the engineered host cell may be recovered and purified so as to form a biocatalyst. The process may include contacting the first alkaloid with an enzyme providing 14-hydroxylation activity in an amount sufficient to convert said first alkaloid to a second alkaloid. In some examples, the first alkaloid may be contacted with a sufficient amount of the one or more enzymes such that at least 5% of said first alkaloid is converted to a second alkaloid. In further examples, the first alkaloid may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said first alkaloid is converted to a second alkaloid.

The one or more enzymes that may be used to convert a first alkaloid to a second alkaloid may contact the first alkaloid in vitro. Additionally, or alternatively, the one or more enzymes that may be used to convert a first alkaloid to a second alkaloid may contact the first alkaloid in vivo. In some examples, the one or more enzymes that may be used to convert a first alkaloid to a second alkaloid may be provided to a cell having the first alkaloid within. In some examples, the one or more enzymes that may be used to convert a first alkaloid to a second alkaloid may be produced within an engineered host cell.

In some examples, the methods provide for engineered host cells that produce an alkaloid product, wherein the 14-hydroxylation of a substrate to a product may comprise a key step in the production of an alkaloid product. In some examples, the alkaloid produced is a nor-opioid. In another embodiments, a first alkaloid is an intermediate toward the product of the engineered host cell. In still other embodiments, the alkaloid product is selected from the group including naloxone, naltrexone, and nalmefene.

In some examples, the substrate alkaloid is an opioid selected from the group consisting of norcodeine, noroxycodone, northebaine, norhydrocodone, nordihydro-codeine, nor-14-hydroxy-codeine, norcodeinone, nor-14-hydroxy-codeinone, normorphine, noroxymorphone, nororipavine, norhydro-morphone, nordihydro-morphine, nor-14-hydroxy-morphine, normorphinone, and nor-14-hydroxy-morphinone. In some examples, the cosubstrate is S-adenosylmethionine, allyl-S-adenosylmethionine, or cyclopropylmethyl-S-adenosylmethionine.

In some embodiments, the methods provide for engineered host cells that produce a BIA product at least 1.1, at least 1.2, at least 1.3, at least 1.4, at least 1.5, at least 1.6, at least 1.7, at least 1.8, at least 1.9, at least about 2, at least about 3, at least about 4, at least about 5,fold more than the same host cell that is not engineered.

In some embodiments, the methods provide for engineered host cells that produce a BIA product at least about 10%, 15%, 20%, 25%, 30%, 35%, 40%, 45%, 50% more than the same host cell that is not engineered.

### Methods

### Methods for Culturing Host Cells for BIA Production

As summarized above, some aspects of the disclosure include methods of preparing benzylisoquinoline alkaloids (BIAs) of interest. Additionally, some aspects of the disclosure include methods of preparing enzymes of interest. As such, some aspects of the disclosure include culturing an engineered host cell under conditions in which the one or more host cell modifications (e.g., as described herein) are functionally expressed such that the cell converts starting compounds of interest into product enzymes and/or BIAs of interest. Also provided are methods that include culturing an engineered host cell under conditions suitable for protein production such that one or more heterologous coding sequences are functionally expressed and convert starting compounds of interest into product enzymes or BIAs of interest. In some examples, the method is a method of preparing a benzylisoquinoline alkaloid (BIA) that includes culturing an engineered host cell (e.g., as described herein); adding a starting compound to the cell culture; and recovering the BIA from the cell culture. In some examples, the method is a method of preparing an enzyme that includes culturing an engineered host cell (e.g., as described herein); adding a starting compound to the cell culture; and recovering the enzyme from the cell culture.

Fermentation media may contain suitable carbon substrates. The source of carbon suitable to perform the methods of this disclosure may encompass a wide variety of carbon containing substrates. Suitable substrates may include, without limitation, monosaccharides (e.g., glucose, fructose, galactose, xylose), oligosaccharides (e.g., lactose, sucrose, raffinose), polysaccharides (e.g., starch, cellulose), or a combination thereof. In some cases, unpurified mixtures from renewable feedstocks may be used (e.g., cornsteep liquor, sugar beet molasses, barley malt). In some cases, the carbon substrate may be a one-carbon substrate (e.g., methanol, carbon dioxide) or a two-carbon substrate (e.g., ethanol). In other cases, other carbon containing compounds may be utilized, for example, methylamine, glucosamine, and amino acids.

Any convenient methods of culturing engineered host cells may be employed for producing the enzymes and/or BIAs of interest. The particular protocol that is employed may vary, e.g., depending on the engineered host cell, the heterologous coding sequences, the enzymes of interest, the BIAs of interest, etc. The engineered host cells may be present in any convenient environment, such as an environment in which the engineered host cells are capable of expressing one or more functional heterologous enzymes. In some embodiments, the engineered host cells are cultured under conditions that are conducive to enzyme expression and with appropriate substrates available to allow production of enzymes and/or BIAs of interest in vivo. In some embodiments, the functional enzymes are extracted from the engineered host for production of enzymes and/or BIAs of interest under in vitro conditions. In some instances, the engineered host cells are placed back into a multicellular host organism. The engineered host cells are in any phase of growth, including, but not limited to, stationary phase and log-growth phase, etc. In addition, the cultures themselves may be continuous cultures or they may be batch cultures.

Cells may be grown in an appropriate fermentation medium at a temperature between 14-40° C. Cells may be grown with shaking at any convenient speed (e.g., 200 rpm). Cells may be grown at a suitable pH. Suitable pH ranges for the fermentation may be between pH 5-9. Fermentations may be performed under aerobic, anaerobic, or microaerobic conditions. Any suitable growth medium may be used. Suitable growth media may include, without limitation, common commercially prepared media such as synthetic defined (SD) minimal media or yeast extract peptone dextrose (YEPD) rich media. Any other rich, defined, or synthetic growth media appropriate to the microorganism may be used.

Cells may be cultured in a vessel of essentially any size and shape. Examples of vessels suitable to perform the methods of this disclosure may include, without limitation, multi-well shake plates, test tubes, flasks (baffled and non-baffled), and bioreactors. The volume of the culture may range from 10 microliters to greater than 10,000 liters.

The addition of agents to the growth media that are known to modulate metabolism in a manner desirable for the production of alkaloids may be included. In a non-limiting example, cyclic adenosine 2′3′-monophosphate may be added to the growth media to modulate catabolite repression.

Any convenient cell culture conditions for a particular cell type may be utilized. In certain embodiments, the host cells that include one or more modifications are cultured under standard or readily optimized conditions, with standard cell culture media and supplements. As one example, standard growth media when selective pressure for plasmid maintenance is not required may contain 20 g/L yeast extract, 10 g/L peptone, and 20 g/L dextrose (YPD). Host cells containing plasmids are grown in synthetic complete (SC) media containing 1.7 g/L yeast nitrogen base, 5 g/L ammonium sulfate, and 20 g/L dextrose supplemented with the appropriate amino acids required for growth and selection. Alternative carbon sources which may be useful for inducible enzyme expression include, but are not limited to, sucrose, raffinose, and galactose. Cells are grown at any convenient temperature (e.g., 30° C.) with shaking at any convenient rate (e.g., 200 rpm) in a vessel, e.g., in test tubes or flasks in volumes ranging from 1-1000 mL, or larger, in the laboratory.

Culture volumes may be scaled up for growth in larger fermentation vessels, for example, as part of an industrial process. The industrial fermentation process may be carried out under closed-batch, fed-batch, or continuous chemostat conditions, or any suitable mode of fermentation. In some cases, the engineered host cells may be immobilized on a substrate as whole cell catalysts and subjected to fermentation conditions for alkaloid production.

A batch fermentation is a closed system, in which the composition of the medium is set at the beginning of the fermentation and not altered during the fermentation process. The desired organism(s) are inoculated into the medium at the beginning of the fermentation. In some instances, the batch fermentation is run with alterations made to the system to control factors such as pH and oxygen concentration (but not carbon). In this type of fermentation system, the biomass and metabolite compositions of the system change continuously over the course of the fermentation. Cells typically proceed through a lag phase, then to a log phase (high growth rate), then to a stationary phase (growth rate reduced or halted), and eventually to a death phase (if left untreated). In additional cases, the batch fermentation system may be opened at certain times to add additional substrates for fermentating the desired organism. In particular, in some cases, a fermentation system may include a fed batch reactor.

A continuous fermentation is an open system, in which a defined fermentation medium is added continuously to the bioreactor and an equal amount of fermentation media is continuously removed from the vessel for processing. Continuous fermentation systems are generally operated to maintain steady state growth conditions, such that cell loss due to medium being removed must be balanced by the growth rate in the fermentation. Continuous fermentations are generally operated at conditions where cells are at a constant high cell density. Continuous fermentations allow for the modulation of one or more factors that affect target product concentration and/or cell growth.

The liquid medium may include, but is not limited to, a rich or synthetic defined medium having an additive component described above. Media components may be dissolved in water and sterilized by heat, pressure, filtration, radiation, chemicals, or any combination thereof. Several media components may be prepared separately and sterilized, and then combined in the fermentation vessel. The culture medium may be buffered to aid in maintaining a constant pH throughout the fermentation.

Process parameters including temperature, dissolved oxygen, pH, stirring, aeration rate, and cell density may be monitored or controlled over the course of the fermentation. For example, temperature of a fermentation process may be monitored by a temperature probe immersed in the culture medium. The culture temperature may be controlled at the set point by regulating the jacket temperature. Water may be cooled in an external chiller and then flowed into the bioreactor control tower and circulated to the jacket at the temperature required to maintain the set point temperature in the vessel.

Additionally, a gas flow parameter may be monitored in a fermentation process. For example, gases may be flowed into the medium through a sparger. Gases suitable for the methods of this disclosure may include compressed air, oxygen, and nitrogen. Gas flow may be at a fixed rate or regulated to maintain a dissolved oxygen set point.

The pH of a culture medium may also be monitored. In some examples, the pH may be monitored by a pH probe that is immersed in the culture medium inside the vessel. If pH control is in effect, the pH may be adjusted by acid and base pumps which add each solution to the medium at the required rate. The acid solutions used to control pH may be sulfuric acid or hydrochloric acid. The base solutions used to control pH may be sodium hydroxide, potassium hydroxide, or ammonium hydroxide.

Further, dissolved oxygen may be monitored in a culture medium by a dissolved oxygen probe immersed in the culture medium. If dissolved oxygen regulation is in effect, the oxygen level may be adjusted by increasing or decreasing the stirring speed. The dissolved oxygen level may also be adjusted by increasing or decreasing the gas flow rate. The gas may be compressed air, oxygen, or nitrogen.

Stir speed may also be monitored in a fermentation process. In some examples, the stirrer motor may drive an agitator. The stirrer speed may be set at a consistent rpm throughout the fermentation or may be regulated dynamically to maintain a set dissolved oxygen level.

Additionally, turbidity may be monitored in a fermentation process. In some examples, cell density may be measured using a turbidity probe. Alternatively, cell density may be measured by taking samples from the bioreactor and analyzing them in a spectrophotometer. Further, samples may be removed from the bioreactor at time intervals through a sterile sampling apparatus. The samples may be analyzed for alkaloids produced by the host cells. The samples may also be analyzed for other metabolites and sugars, the depletion of culture medium components, or the density of cells.

In another example, a feed stock parameter may be monitored during a fermentation process. In particular, feed stocks including sugars and other carbon sources, nutrients, and cofactors that may be added into the fermentation using an external pump. Other components may also be added during the fermentation including, without limitation, anti-foam, salts, chelating agents, surfactants, and organic liquids.

Any convenient codon optimization techniques for optimizing the expression of heterologous polynucleotides in host cells may be adapted for use in the subject host cells and methods, see, e.g., Gustafsson, C. et al. (2004) Trends Biotechnol, 22, 346-353.

The subject method may also include adding a starting compound to the cell culture. Any convenient methods of addition may be adapted for use in the subject methods. The cell culture may be supplemented with a sufficient amount of the starting materials of interest (e.g., as described herein), e.g., a mM to M amount such as between about 1-5 mM of a starting compound. It is understood that the amount of starting material added, the timing and rate of addition, the form of material added, etc., may vary according to a variety of factors. The starting material may be added neat or pre-dissolved in a suitable solvent (e.g., cell culture media, water, or an organic solvent). The starting material may be added in concentrated form (e.g., 10× over desired concentration) to minimize dilution of the cell culture medium upon addition. The starting material may be added in one or more batches, or by continuous addition over an extended period of time (e.g., hours or days).

Methods for Isolating Products from the Fermentation Medium

The subject methods may also include recovering the enzymes and/or BIAs of interest from the cell culture. Any convenient methods of separation and isolation (e.g., chromatography methods or precipitation methods) may be adapted for use in the subject methods to recover the enzymes and/or BIAs of interest from the cell culture. Filtration methods may be used to separate soluble from insoluble fractions of the cell culture. In some cases, liquid chromatography methods (e.g., reverse phase HPLC, size exclusion, normal phase chromatography) may be used to separate the BIA of interest from other soluble components of the cell culture. In some cases, extraction methods (e.g., liquid extraction, pH based purification, solid phase extraction, affinity chromatography, ion exchange, etc.) may be used to separate the enzymes and/or BIAs of interest from other components of the cell culture.

The produced alkaloids may be isolated from the fermentation medium using methods known in the art. A number of recovery steps may be performed immediately after (or in some instances, during) the fermentation for initial recovery of the desired product. Through these steps, the alkaloids (e.g., BIAs) may be separated from the engineered host cells, cellular debris and waste, and other nutrients, sugars, and organic molecules may remain in the spent culture medium. This process may be used to yield a BIA-enriched product.

In an example, a product stream having a benzylisoquinoline alkaloid (BIA) product is formed by providing engineered yeast cells and a feedstock including nutrients and water to a batch reactor. In particular, the engineered yeast cells may be subjected to fermentation by incubating the engineered yeast cells for a time period of at least about 5 minutes to produce a solution comprising the BIA product and cellular material. Once the engineered yeast cells have been subjected to fermentation, at least one separation unit may be used to separate the BIA product from the cellular material to provide the product stream comprising the BIA product. In particular, the product stream may include the BIA product as well as additional components, such as a clarified yeast culture medium. Additionally, a BIA product may comprise one or more BIAs of interest, such as one or more BIA compounds.

Different methods may be used to remove cells from a bioreactor medium that include an enzyme and/or BIA of interest. In some examples, cells may be removed by sedimentation over time. This process of sedimentation may be accelerated by chilling or by the addition of fining agents such as silica. The spent culture medium may then be siphoned from the top of the reactor or the cells may be decanted from the base of the reactor. Alternatively, cells may be removed by filtration through a filter, a membrane, or other porous material. Cells may also be removed by centrifugation, for example, by continuous flow centrifugation or by using a continuous extractor.

If some valuable enzymes and/or BIAs of interest are present inside the engineered host cells, the engineered host cells may be permeabilized or lysed and the cell debris may be removed by any of the methods described above. Agents used to permeabilize the engineered host cells may include, without limitation, organic solvents (e.g., DMSO) or salts (e.g., lithium acetate). Methods to lyse the engineered host cells may include the addition of surfactants such as sodium dodecyl sulfate, or mechanical disruption by bead milling or sonication.

Enzymes and/or BIAs of interest may be extracted from the clarified spent culture medium through liquid-liquid extraction by the addition of an organic liquid that is immiscible with the aqueous culture medium. In some examples, the use of liquid-liquid extraction may be used in addition to other processing steps. Examples of suitable organic liquids include, but are not limited to, isopropyl myristate, ethyl acetate, chloroform, butyl acetate, methylisobutyl ketone, methyl oleate, toluene, oleyl alcohol, ethyl butyrate. The organic liquid may be added to as little as 10% or as much as 100% of the aqueous medium. The organic liquid may be as little as 10%, may be 100%, may be 200%, may be 300%, may be 400%, may be 500%, may be 600%, may be 700%, may be 800%, may be 900%, may be 1000%, may be more than 1000%, or may be a percentage in between those listed herein of the volume of the aqueous liquid.

In some cases, the organic liquid may be added at the start of the fermentation or at any time during the fermentation. This process of extractive fermentation may increase the yield of enzymes and/or BIAs of interest from the host cells by continuously removing enzymes and/or BIAs to the organic phase.

Agitation may cause the organic phase to form an emulsion with the aqueous culture medium. Methods to encourage the separation of the two phases into distinct layers may include, without limitation, the addition of a demulsifier or a nucleating agent, or an adjustment of the pH. The emulsion may also be centrifuged to separate the two phases, for example, by continuous conical plate centrifugation.

Alternatively, the organic phase may be isolated from the aqueous culture medium so that it may be physically removed after extraction. For example, the solvent may be encapsulated in a membrane.

In some examples, enzymes and/or BIAs of interest may be extracted from a fermentation medium using adsorption methods. In some examples, BIAs of interest may be extracted from clarified spent culture medium by the addition of a resin such as Amberlite® XAD4 or another agent that removes BIAs by adsorption. The BIAs of interest may then be released from the resin using an organic solvent. Examples of suitable organic solvents include, but are not limited to, methanol, ethanol, ethyl acetate, or acetone.

BIAs of interest may also be extracted from a fermentation medium using filtration. At high pH, the BIAs of interest may form a crystalline-like precipitate in the bioreactor. This precipitate may be removed directly by filtration through a filter, membrane, or other porous material. The precipitate may also be collected by centrifugation and/or decantation.

The extraction methods described above may be carried out either in situ (in the bioreactor) or ex situ (e.g., in an external loop through which media flows out of the bioreactor and contacts the extraction agent, then is recirculated back into the vessel). Alternatively, the extraction methods may be performed after the fermentation is terminated using the clarified medium removed from the bioreactor vessel.

Methods for Purifying Products from Alkaloid-Enriched Solutions

Subsequent purification steps may involve treating the post-fermentation solution enriched with BIA product(s) of interest using methods known in the art to recover individual product species of interest to high purity.

In one example, BIAs of interest extracted in an organic phase may be transferred to an aqueous solution. In some cases, the organic solvent may be evaporated by heat and/or vacuum, and the resulting powder may be dissolved in an aqueous solution of suitable pH. In a further example, the BIAs of interest may be extracted from the organic phase by addition of an aqueous solution at a suitable pH that promotes extraction of the BIAs of interest into the aqueous phase. The aqueous phase may then be removed by decantation, centrifugation, or another method.

The BIA-containing solution may be further treated to remove metals, for example, by treating with a suitable chelating agent. The BIA of interest-containing solution may be further treated to remove other impurities, such as proteins and DNA, by precipitation. In one example, the BIA of interest-containing solution is treated with an appropriate precipitation agent such as ethanol, methanol, acetone, or isopropanol. In an alternative example, DNA and protein may be removed by dialysis or by other methods of size exclusion that separate the smaller alkaloids from contaminating biological macromolecules.

In further examples, the solution containing BIAs of interest may be extracted to high purity by continuous cross-flow filtration using methods known in the art.

If the solution contains a mixture of BIAs of interest, it may be subjected to acid-base treatment to yield individual BIA of interest species using methods known in the art. In this process, the pH of the aqueous solution is adjusted to precipitate individual BIAs.

For high purity, small-scale preparations, the BIAs may be purified in a single step by liquid chromatography.

### Liquid Chromatography Mass Spectrometry (LCMS) Method

The BIA compounds of interest, including 1-benzylisoquinoline alkaloids, bisbenzylisoquinoline alkaloids, promorphinan alkaloids, morphinan alkaloids, nal-opioids, and nor-opioids, may be separated using liquid chromatography, and detected and quantified using mass spectrometry. Compound identity may be confirmed by characteristic elution time, mass-to-charge ratio (m/z) and fragmentation patterns (MS/MS). Quantitation may be performed by comparison of compound peak area to a standard curve of a known reference standard compound. Additionally, BIAs of interest may be detected by alternative methods such as GC-MS, UV-vis spectroscopy, NMR, LC-NMR, LC-UV, TLC, and capillary electrophoresis.

### Purpald Assay Method

For high throughput screening of demethylation reactions a purpald assay may be used. For example, demethylation catalyzed by 2-oxoglutarate dependent dioxygenases produces formaldehyde as product as shown in the generalized chemical equation: [substrate]+2-oxoglutarate+O₂: [product]+formaldehyde+succinate+CO₂. Purpald reagent in alkaline conditions undergoes a color change in the presence of formaldehyde that can be quantified to concentrations as low as 1 nM with a spectrophotometer at 510 nm.

### Yeast-Derived Alkaloid APIs Versus Plant-Derived APIs

The clarified yeast culture medium (CYCM) may contain a plurality of impurities. The clarified yeast culture medium may be dehydrated by vacuum and/or heat to yield an alkaloid-rich powder. This product is analogous to the concentrate of poppy straw (CPS) or opium, which is exported from poppy-growing countries and purchased by API manufacturers. For the purposes of this disclosure, CPS is a representative example of any type of purified plant extract from which the desired alkaloids product(s) may ultimately be further purified. Tables 14 and 15 highlight the impurities in these two products that may be specific to either CYCM or CPS or may be present in both. While some BIAs may have a pigment as an impurity, other BIAs may be categorized as pigments themselves. Accordingly, these BIAs may be assessed for impurities based on non-pigment impurities. By analyzing a product of unknown origin for a subset of these impurities, a person of skill in the art could determine whether the product originated from a yeast or plant production host.

API-grade pharmaceutical ingredients are highly purified molecules. As such, impurities that could indicate the plant- or yeast-origin of an API (such as those listed in Tables 14 and 15) may not be present at the API stage of the product. Indeed, many of the API products derived from yeast strains of some embodiments of the present disclosure may be largely indistinguishable from the traditional plant-derived APIs. In some cases, however, conventional alkaloid compounds may be subjected to chemical modification using chemical synthesis approaches, which may show up as chemical impurities in plant-based products that require such chemical modifications. For example, chemical derivatization may often result in a set of impurities related to the chemical synthesis processes. In certain situations, these modifications may be performed biologically in the yeast production platform, thereby avoiding some of the impurities associated with chemical derivation from being present in the yeast-derived product. In particular, these impurities from the chemical derivation product may be present in an API product that is produced using chemical synthesis processes but may be absent from an API product that is produced using a yeast-derived product. Alternatively, if a yeast-derived product is mixed with a chemically-derived product, the resulting impurities may be present but in a lesser amount than would be expected in an API that only or primarily contains chemically-derived products. In this example, by analyzing the API product for a subset of these impurities, a person of skill in the art could determine whether the product originated from a yeast production host or the traditional chemical derivatization route.

Non-limiting examples of impurities that may be present in chemically-derivatized morphinan APIs but not in biosynthesized APIs include a codeine-O(6)-methyl ether impurity in API codeine; 8,14-dihydroxy-7,8-dihydrocodeinone in API oxycodone; and tetrahydrothebaine in API hydrocodone. The codeine-O(6)-methyl ether may be formed by chemical over-methylation of morphine. The 8,14-dihydroxy-7,8-dihydrocodeinone in API oxycodone may be formed by chemical over-oxidation of thebaine. Additionally, the tetrahydrothebaine in API hydrocodone may be formed by chemical over-reduction of thebaine.

However, in the case where the yeast-derived compound and the plant-derived compound are both subjected to chemical modification through chemical synthesis approaches, the same impurities associated with the chemical synthesis process may be expected in the products. In such a situation, the starting material (e.g., CYCM or CPS) may be analyzed as described above.

### Host Cell Derived Nal-Opioids Vs Chemically Derived Nal-Opioids

Nal-opioids produced by chemical synthesis may contain a plurality of impurities. These impurities may arise from many different causes, for example, unreacted starting materials, incomplete reactions, the formation of byproducts, persistence of intermediates, dimerization, or degradation. An example of an unreacted starting material could be oxymorphone remaining in a preparation of naltrexone. An example of an impurity arising from an incomplete reaction could be 3-O-Methylbuprenorphine resulting from the incomplete 3-O-demethylation of thebaine. Chemical modification can result in the addition or removal of functional groups at off-target sites. For example, the oxidation of C10 to create 10-hydroxynaltrexone and 10-ketonaltrexone during naltrexone synthesis, or the removal of the 6-O-methyl group to give 6-O-desmethylbuprenorphine during buprenorphine synthesis. Impurites may arise from the persistence of reaction intermediates, for example the persistence of N-oxides like oxymorphone N-oxide formed during the N-demethylation process. Another source of impurities is dimerization, the conjugation of two opioid molecules, for example two buprenorphine molecules (2,2′-bisbuprenorphine), two naltrexone molecules (2,2′-bisnaltrexone), or two naloxone molecules (2,2′-bisnaloxone). Impurities may arise from degradation of starting materials, reaction intermediates, or reaction products. The extreme physical conditions used in chemical syntheses may make the presence of degradation more likely. An example of an impurity that may arise from degradation is dehydrobuprenorphine produced by oxidizing conditions during buprenorphine synthesis.

Nal-opioids produced by enzyme catalysis in a host cell may contain different impurities than nal-opioids produced by chemical synthesis. Nal-opioids produced by enzyme catalysis in a host cell may contain fewer impurities than nal-opioids produced by chemical synthesis. Nal-opioids produced by enzyme catalysis in a host cell may lack certain impurities that are found in nal-opioids produced by chemical synthesis. In some examples, key features of enzyme synthesis may include, (1) enzymes target a specific substrate and residue with high fidelity; (2) enzymes perform reactions in the mild physiological conditions within the cell which do not compromise the stability of the molecules; and (3) enzymes are engineered to be efficient catalysts that drive reactions to completion.

Table 16 highlights some of the impurities that may be specific to chemically produced nal-opioids. Accordingly, nal-opioids may be assessed for impurities to determine the presence or absence of any impurity from Table 16. By analyzing a product of unknown origin for a subset of these impurities, a person of skill in the art could determine whether the product originated from a chemical or enzymatic synthesis.

### Methods of Engineering Host Cells

Also included are methods of engineering host cells for the purpose of producing enzymes and/or BIAs of interest. Inserting DNA into host cells may be achieved using any convenient methods. The methods are used to insert the heterologous coding sequences into the engineered host cells such that the host cells functionally express the enzymes and convert starting compounds of interest into product enzymes and/or BIAs of interest.

Any convenient promoters may be utilized in the subject engineered host cells and methods. The promoters driving expression of the heterologous coding sequences may be constitutive promoters or inducible promoters, provided that the promoters are active in the engineered host cells. The heterologous coding sequences may be expressed from their native promoters, or non-native promoters may be used. Such promoters may be low to high strength in the host in which they are used. Promoters may be regulated or constitutive. In certain embodiments, promoters that are not glucose repressed, or repressed only mildly by the presence of glucose in the culture medium, are used. Promoters of interest include but are not limited to, promoters of glycolytic genes such as the promoter of the B. subtilis tsr gene (encoding the promoter region of the fructose bisphosphate aldolase gene) or the promoter from yeast S. cerevisiae gene coding for glyceraldehyde 3-phosphate dehydrogenase (GPD, GAPDH, or TDH3), the ADH1 promoter of baker's yeast, the phosphate-starvation induced promoters such as the PHO5 promoter of yeast, the alkaline phosphatase promoter from B. licheniformis, yeast inducible promoters such as Gal1-10, Gal1, GalL, GalS, repressible promoter Met25, tetO, and constitutive promoters such as glyceraldehyde 3-phosphate dehydrogenase promoter (GPD), alcohol dehydrogenase promoter (ADH), translation-elongation factor-1-α promoter (TEF), cytochrome c-oxidase promoter (CYC1), MRP7 promoter, etc. Autonomously replicating yeast expression vectors containing promoters inducible by hormones such as glucocorticoids, steroids, and thyroid hormones may also be used and include, but are not limited to, the glucorticoid responsive element (GRE) and thyroid hormone responsive element (TRE). These and other examples are described in U.S. Pat. No. 7,045,290, which is incorporated by reference, including the references cited therein. Additional vectors containing constitutive or inducible promoters such as a factor, alcohol oxidase, and PGH may be used. Additionally any promoter/enhancer combination (as per the Eukaryotic Promoter Data Base EPDB) could also be used to drive expression of genes. Any convenient appropriate promoters may be selected for the host cell, e.g., E. coli. One may also use promoter selection to optimize transcript, and hence, enzyme levels to maximize production while minimizing energy resources.

Any convenient vectors may be utilized in the subject engineered host cells and methods. Vectors of interest include vectors for use in yeast and other cells. The types of yeast vectors may be broken up into 4 general categories: integrative vectors (YIp), autonomously replicating high copy-number vectors (YEp or 2 plasmids), autonomously replicating low copy-number vectors (YCp or centromeric plasmids) and vectors for cloning large fragments (YACs). Vector DNA is introduced into prokaryotic or eukaryotic cells via any convenient transformation or transfection techniques. DNA of another source (e.g. PCR-generated double stranded DNA product, or synthesized double stranded or single stranded oligonucleotides) may be used to engineer the yeast by integration into the genome. Any single transformation event may include one or several nucleic acids (vectors, double stranded or single stranded DNA fragments) to genetically modify the host cell. Table 10 illustrates examples of convenient vectors.

### Utility

The engineered host cells and methods of the disclosure, e.g., as described above, find use in a variety of applications. Applications of interest include, but are not limited to: research applications and therapeutic applications. Methods of the disclosure find use in a variety of different applications including any convenient application where the production of enzymes and/or BIAs is of interest.

The subject engineered host cells and methods find use in a variety of therapeutic applications. Therapeutic applications of interest include those applications in which the preparation of pharmaceutical products that include BIAs is of interest. The engineered host cells described herein produce BIAs of interest and enzymes of interest. Reticuline is a major branch point intermediate of interest in the synthesis of BIAs including engineering efforts to produce end products such as opioid products. The subject host cells may be utilized to produce BIAs of interest from simple and inexpensive starting materials that may find use in the production of BIAs of interest, including reticuline, and BIA end products. As such, the subject host cells find use in the supply of therapeutically active BIAs of interest.

In some instances, the engineered host cells and methods find use in the production of commercial scale amounts of BIAs thereof where chemical synthesis of these compounds is low yielding and not a viable means for large-scale production. In certain cases, the host cells and methods are utilized in a fermentation facility that would include bioreactors (fermenters) of, e.g., 5,000-200,000 liter capacity allowing for rapid production of BIAs of interest thereof for therapeutic products. Such applications may include the industrial-scale production of BIAs of interest from fermentable carbon sources such as cellulose, starch, and free sugars.

The subject engineered host cells and methods find use in a variety of research applications. The subject host cells and methods may be used to analyze the effects of a variety of enzymes on the biosynthetic pathways of a variety of enzymes and/or BIAs of interest. In addition, the engineered host cells may be engineered to produce enzymes and/or BIAs of interest that find use in testing for bioactivity of interest in as yet unproven therapeutic functions. In some cases, the engineering of host cells to include a variety of heterologous coding sequences that encode for a variety of enzymes elucidates the high yielding biosynthetic pathways towards enzymes and/or BIAs of interest. In certain cases, research applications include the production of enzymes and/or BIAs of interest for therapeutic molecules of interest that may then be further chemically modified or derivatized to desired products or for screening for increased therapeutic activities of interest. In some instances, host cell strains are used to screen for enzyme activities that are of interest in such pathways, which may lead to enzyme discovery via conversion of BIA metabolites produced in these strains.

The subject engineered host cells and methods may be used as a production platform for plant specialized metabolites. The subject host cells and methods may be used as a platform for drug library development as well as plant enzyme discovery. For example, the subject engineered host cells and methods may find use in the development of natural product based drug libraries by taking yeast strains producing interesting scaffold molecules, such as guattegaumerine, and further functionalizing the compound structure through combinatorial biosynthesis or by chemical means. By producing drug libraries in this way, any potential drug hits are already associated with a production host that is amenable to large-scale culture and production. As another example, these subject engineered host cells and methods may find use in plant enzyme discovery. The subject host cells provide a clean background of defined metabolites to express plant EST libraries to identify new enzyme activities. The subject host cells and methods provide expression methods and culture conditions for the functional expression and increased activity of plant enzymes in yeast.

### Kits and Systems

Aspects of the disclosure further include kits and systems, where the kits and systems may include one or more components employed in methods of the disclosure, e.g., engineered host cells, starting compounds, heterologous coding sequences, vectors, culture medium, etc., as described herein. In some embodiments, the subject kit includes an engineered host cell (e.g., as described herein), and one or more components selected from the following: starting compounds, a heterologous coding sequence and/or a vector including the same, vectors, growth feedstock, components suitable for use in expression systems (e.g., cells, cloning vectors, multiple cloning sites (MCS), bi-directional promoters, an internal ribosome entry site (IRES), etc.), and a culture medium.

Any of the components described herein may be provided in the kits, e.g., host cells including one or more modifications, starting compounds, culture medium, etc. A variety of components suitable for use in making and using heterologous coding sequences, cloning vectors and expression systems may find use in the subject kits. Kits may also include tubes, buffers, etc., and instructions for use. The various reagent components of the kits may be present in separate containers, or some or all of them may be pre-combined into a reagent mixture in a single container, as desired.

Also provided are systems for producing enzymes and/or BIAs of interest, where the systems may include engineered host cells including one or more modifications (e.g., as described herein), starting compounds, culture medium, a fermenter and fermentation equipment, e.g., an apparatus suitable for maintaining growth conditions for the host cells, sampling and monitoring equipment and components, and the like. A variety of components suitable for use in large scale fermentation of yeast cells may find use in the subject systems.

In some cases, the system includes components for the large scale fermentation of engineered host cells, and the monitoring and purification of enzymes and/or BIA compounds produced by the fermented host cells. In certain embodiments, one or more starting compounds (e.g., as described herein) are added to the system, under conditions by which the engineered host cells in the fermenter produce one or more desired BIA products of interest. In some instances, the host cells produce a BIA of interest (e.g., as described herein). In certain cases, the BIA products of interest are opioid products, such as thebaine, codeine, neopine, morphine, neomorphine, hydrocodone, oxycodone, hydromorphone, dihydrocodeine, 14-hydroxycodeine, dihydromorphine, and oxymorphone. In some cases, the BIA products of interest are nal-opioids, such as naltrexone, naloxone, nalmefene, nalorphine, nalorphine, nalodeine, naldemedine, naloxegol, 60-naltrexol, naltrindole, methylnaltrexone, methylsamidorphan, alvimopan, axelopran, bevenpran, dinicotinate, levallorphan, samidorphan, buprenorphine, dezocine, eptazocine, butorphanol, levorphanol, nalbuphine, pentazocine, phenazocine, norbinaltorphimine, and diprenorphine. In some cases, the BIA products of interest are nor-opioids, such as norcodeine, noroxycodone, northebaine, norhydrocodone, nordihydro-codeine, nor-14-hydroxy-codeine, norcodeinone, nor-14-hydroxy-codeinone, normorphine, noroxymorphone, nororipavine, norhydro-morphone, nordihydro-morphine, nor-14-hydroxy-morphine, normorphinone, and nor-14-hydroxy-morphinone. In some cases, the BIA products are bisbenzylisoquinoline products, such as berbamunine, guattegaumerine, dauricine, and liensinine.

In some cases, the system includes processes for monitoring and or analyzing one or more enzymes and/or BIAs of interest compounds produced by the subject host cells. For example, a LC-MS analysis system as described herein, a chromatography system, or any convenient system where the sample may be analyzed and compared to a standard, e.g., as described herein. The fermentation medium may be monitored at any convenient times before and during fermentation by sampling and analysis. When the conversion of starting compounds to enzymes and/or BIA products of interest is complete, the fermentation may be halted, and purification of the BIA products may be done. As such, in some cases, the subject system includes a purification component suitable for purifying the enzymes and/or BIA products of interest from the host cell medium into which it is produced. The purification component may include any convenient process that may be used to purify the enzymes and/or BIA products of interest produced by fermentation, including but not limited to, silica chromatography, reverse-phase chromatography, ion exchange chromatography, HIC chromatography, size exclusion chromatography, liquid extraction, and pH extraction methods. In some cases, the subject system provides for the production and isolation of enzyme and/or BIA fermentation products of interest following the input of one or more starting compounds to the system.

The following examples are put forth so as to provide those of ordinary skill in the art with a complete disclosure and description of how to make and use the present disclosure, and are not intended to limit the scope of what the inventors regard as their disclosure nor are they intended to represent that the experiments below are all or the only experiments performed. Efforts have been made to ensure accuracy with respect to numbers used (e.g. amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Discussion of Enzyme List

The host cells may be engineered to include one or more modifications (such as two or more, three or more, four or more, five or more, or even more modifications) that provide for the production of BIAs of interest and/or enzymes of interest. Tables 11 and 17 provide a list of exemplary genes that may be acted upon by one or more modifications so as to provide for the production of BIAs of interest and/or enzymes of interest in an engineered host cell.

Modifications of genes as provided in Tables 11 and 17 may be used to produce BIAs of interest from engineered host cells that are supplied with a medium containing the minimal nutrients required for growth. This minimal medium may contain a carbon source, a nitrogen source, amino acids, vitamins, and salts. For example, modifications of genes as provided in Tables 11 and 17 may be used to produce BIAs of interest from engineered host cells that are fed sugar. Additionally, modifications of one or more genes as provided in Tables 11 and 17 may be used to augment the biosynthetic processes of host cells that may be engineered for drug production.

Additionally, the use of these modifications to provide for the production of BIAs of interest and/or enzymes of interest in engineered host cells is not readily apparent from the mere identification of enzymes that may be produced by the genes. In particular, synthetic pathways that have been reconstructed in host cells, such as yeast cells, as described herein comprise a variety of enzymes that do not act together in nature within a single organism. Additionally, some of the enzymes discussed herein do not act for BIA biosynthesis in their natural context. Further, some of the enzymes described herein are not evolved to function in particular host cells, such as yeast cells, and are not evolved to function together. In these cases, it would not be obvious that the enzymes would exhibit sufficient activity in the context of the synthetic BIA pathway in a host cell, such as yeast, to have sufficient flux through the pathway to produce downstream BIA end products.

For example, plant enzymes are often difficult to functionally express in heterologous microbial hosts, such as yeast. In many cases the enzymes may be misfolded, not correctly localized within the host cell, and/or incorrectly processed. The differences in protein translation and processing between yeast and plants can lead to these enzymes exhibiting substantially reduced to no detectable activities in the yeast host. These challenges arise commonly for endomembrane localized enzymes, such as cytochrome P450s, which are strongly represented in the BIA pathways. Even reduced enzyme activities may pose a substantial challenge to engineering yeast to produce complex BIAs, which requires sufficient activity at each step to ensure high-level accumulation of the desired BIA products.

Additionally, there are endogenous enzymes/pathways in some host cells, such as yeast, that may act on many of the early precursors in the BIA pathway (i.e., intermediates from tyrosine to norcoclaurine), and thus it may not be readily apparent that there would be sufficient flux through the heterologous pathway to achieve substantial BIA production given these competing endogenous pathways. For example, the Erlich pathway (Hazelwood, et al. 2008. Appl. Environ. Microbiol. 74: 2259-66; Larroy, et al. 2003. Chem. Biol. Interact. 143-144: 229-38; Larroy, et al. 2002. Eur. J. Biochem. 269: 5738-45) in yeast is the main endogenous pathway that would act to convert many of the intermediates in the early BIA pathway to undesired products and divert flux from the synthetic pathway.

Further, many of the enzymes as discussed herein, and as provided in Tables 11 and 17, may function under very specific regulation strategies, including spatial regulation, in the native plant hosts, which may be lost upon transfer to the heterologous yeast host. In addition, plants present very different biochemical environments than yeast cells under which the enzymes are evolved to function, including pH, redox state, and substrate, cosubstrate, coenzyme, and cofactor availabilities. Given the differences in biochemical environments and regulatory strategies between the native hosts and the heterologous yeast hosts, it is not obvious that the enzymes would exhibit substantial activities when in the context of the yeast environment and further not obvious that they would work together to direct simple precursors such as sugar to complex BIA compounds. Maintaining the activities of the enzymes in the yeast host is particularly important as many of the pathways have many reaction steps (>10), such that if these steps are not efficient then one would not expect accumulation of desired downstream products.

In addition, in the native plant hosts, the associated metabolites in these pathways may be localized across different cell and tissue types. In several examples, there are cell types that may be specialized for biosynthesis and cell types that may be synthesized for metabolite accumulation. This type of cell specialization may be lost when expressing the pathways within a heterologous yeast host, and may play an important role in controlling the toxicity of these metabolites on the cells. Thus, it is not obvious that yeast could be successfully engineered to biosynthesize and accumulate these metabolites without being harmed by the toxicity of these compounds.

As one example, in the native plant hosts, the enzyme BBE is reported to have dynamic subcellular localization. In particular, the enzyme BBE initially starts in the ER and then is sorted to the vacuole (Bird and Facchini. 2001. Planta. 213: 888-97). It has been suggested that the ER-association of BBE in plants (Alcantara, et al. 2005. Plant Physiol. 138: 173-83) provides the optimal basic pH (pH ˜8.8) for BBE activity (Ziegler and Facchini. 2008. Annu. Rev. Plant Biol. 59: 735-69). As another example, there is evidence that sanguinarine biosynthesis occurs in specialized vesicles within plant cells (Amann, et al. 1986. Planta. 167: 310-20), but only some of the intermediates accumulate in the vesicles. This may occur so as to sequester them from other enzyme activities and/or toxic effects.

As another example, the biosynthetic enzymes in the morphinan pathway branch are all localized to the phloem, which is part of the vascular tissue in plants. In the phloem, the pathway enzymes may be further divided between two cell types: the sieve elements common to all plants, and the laticifer which is a specialized cell type present only in certain plants which make specialized secondary metabolites. The upstream enzymes (i.e., from NCS through to SalAT) are predominantly in the sieve elements, and the downstream enzymes (i.e., T6ODM, COR, CODM) are mostly in the laticifer (Onoyovwe, et al. 2013. Plant Cell. 25: 4110-22). Additionally, it was discovered that the final steps in the noscapine biosynthetic pathway take place in the laticifer (Chen and Facchini. 2014. Plant J. 77: 173-84). This compartmentalization is thought to be highly important for regulating biosynthesis by isolating or trafficking intermediates, providing optimal pH, enhancing supply of cofactors, although the nature of the poppy laticifer microenvironment is still under investigation (Ziegler and Facchini. 2008. Annu. Rev. Plant Biol. 59: 735-69). Further, it is predicted that several of the enzymes may function as multi-enzyme complexes or metabolic channels common to plant secondary metabolism (Kempe, et al. 2009. Phytochemistry. 70: 579-89; Allen, et al. 2004. Nat. Biotechnol. 22: 1559-66). When biosynthetic enzymes are combined from different hosts and/or expressed recombinantly in a heterologous yeast cell it is not clear that these complexes or channels will form as they would in the native host. In an additional example, in Coptis japonica, berberine is biosynthesized in root tissues and then accumulated within the rhizome via the action of specialized ATP-binding cassette transport proteins (Shitan, et al. 2013. Phytochemistry. 91: 109-16). In opium poppy, morphinan alkaloids are accumulated within the latex (cytoplasm of laticifer cells) (Martin, et al. 1967. Biochemistry. 6: 2355-63).

Further, even without these considerations, it is also the case that the plant enzymes for several of the steps in the pathways described herein have not yet been characterized. For example, the conversion of tyrosine to the early benzylisoquinoline alkaloid scaffold norcoclaurine has not yet been characterized. Thus, for several of the steps in the pathways described herein, alternative biosynthetic scheme were produced by bringing together enzyme activities that do not normally occur together in nature for the biosynthesis of BIAs or identifying new enzyme activities from genome sequence information to use in the reconstructed pathways.

For example, the two-step conversion of tyrosine to dopamine may be achieved by combining at least 5 mammalian enzymes and 1 bacterial enzyme, which do not naturally occur together and were not evolved to function in the context of this pathway or with plant enzymes. In these instances, it may not be obvious to utilize these enzymes for the biosynthesis of compounds they were not evolved for in nature and that they would function effectively in the context of a heterologous microbial host and this pathway.

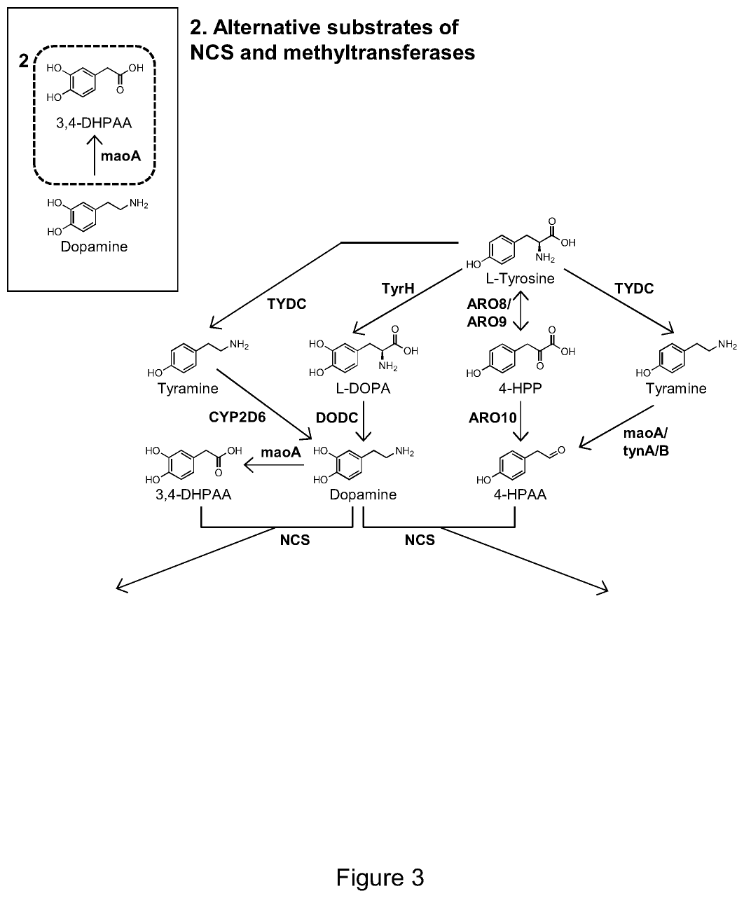

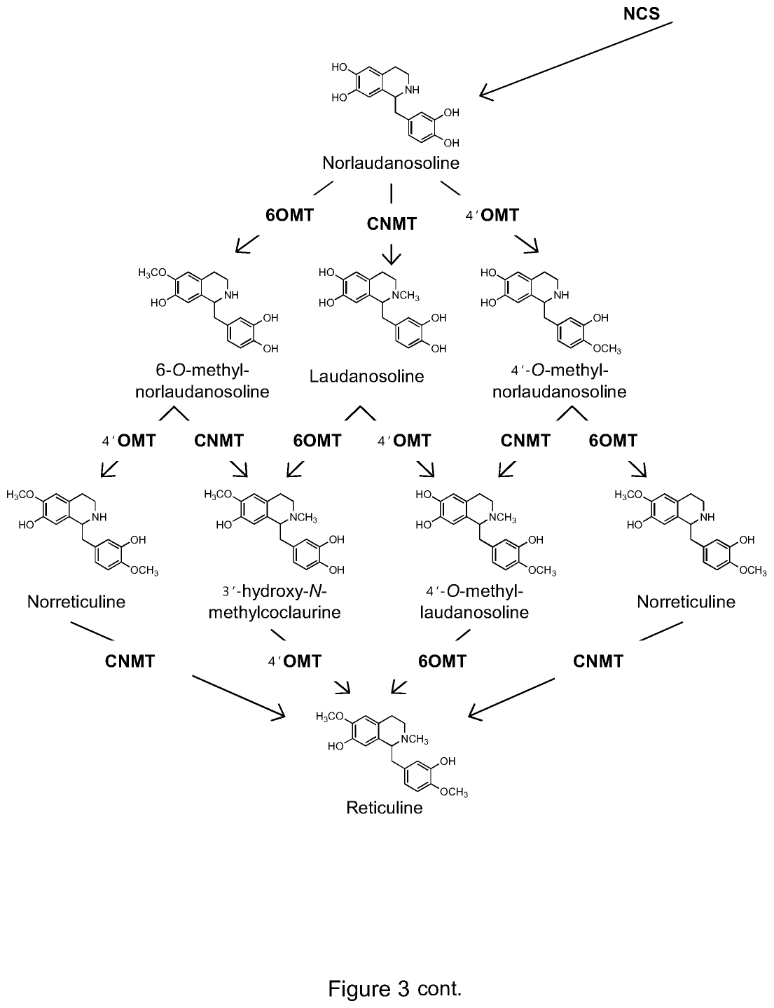

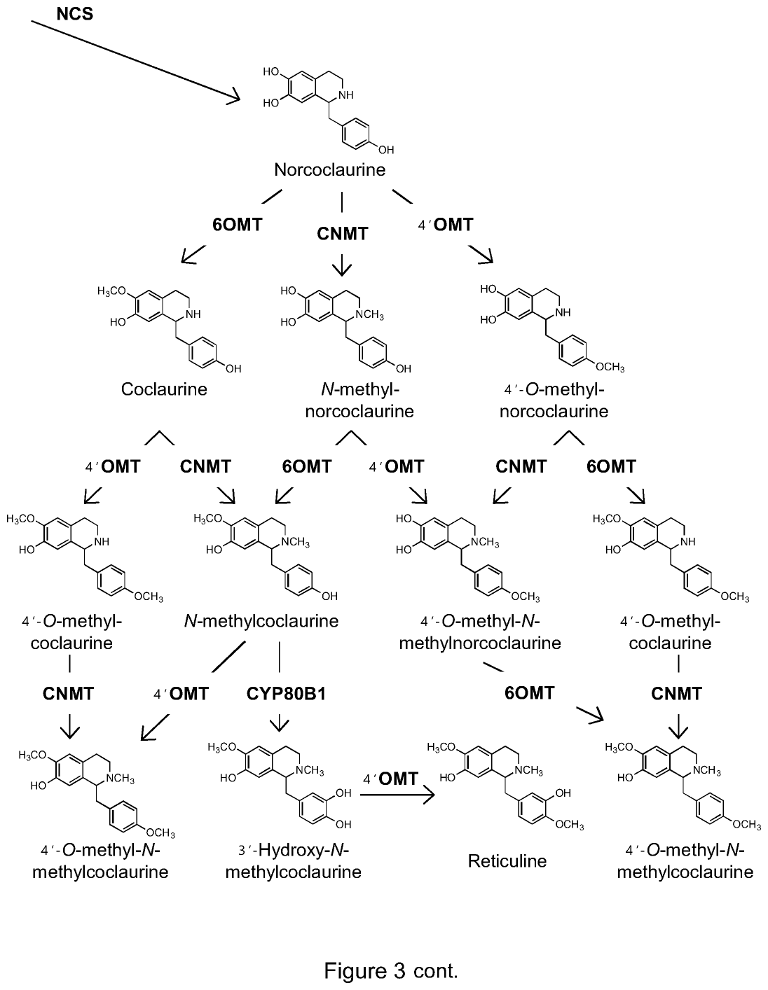

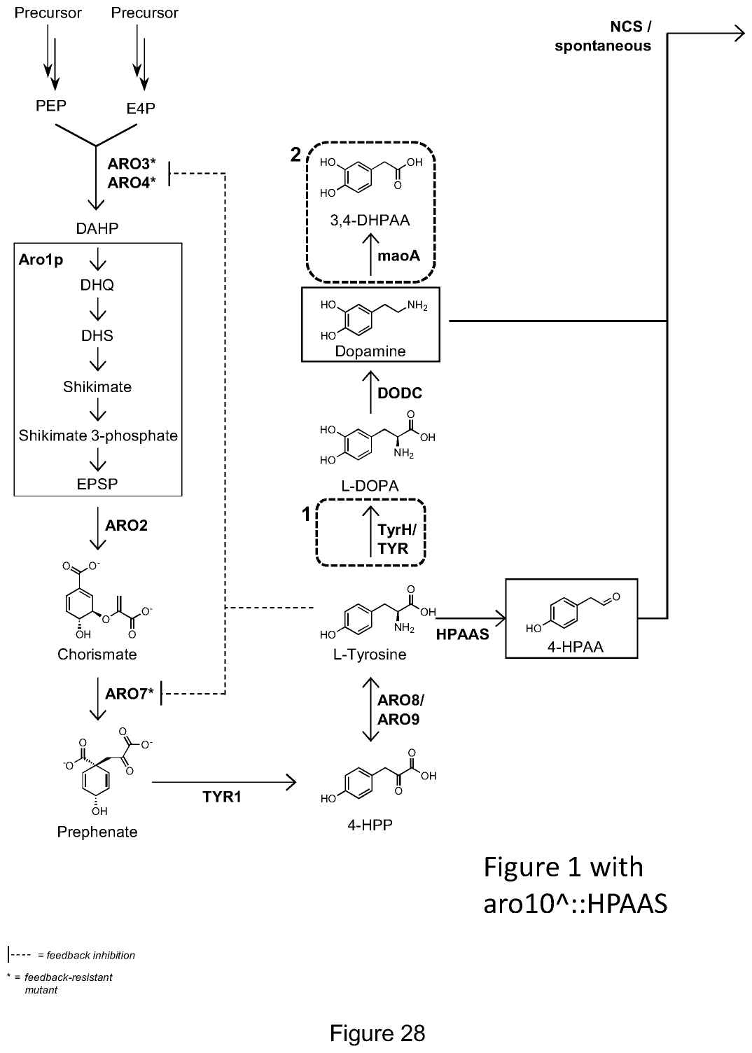

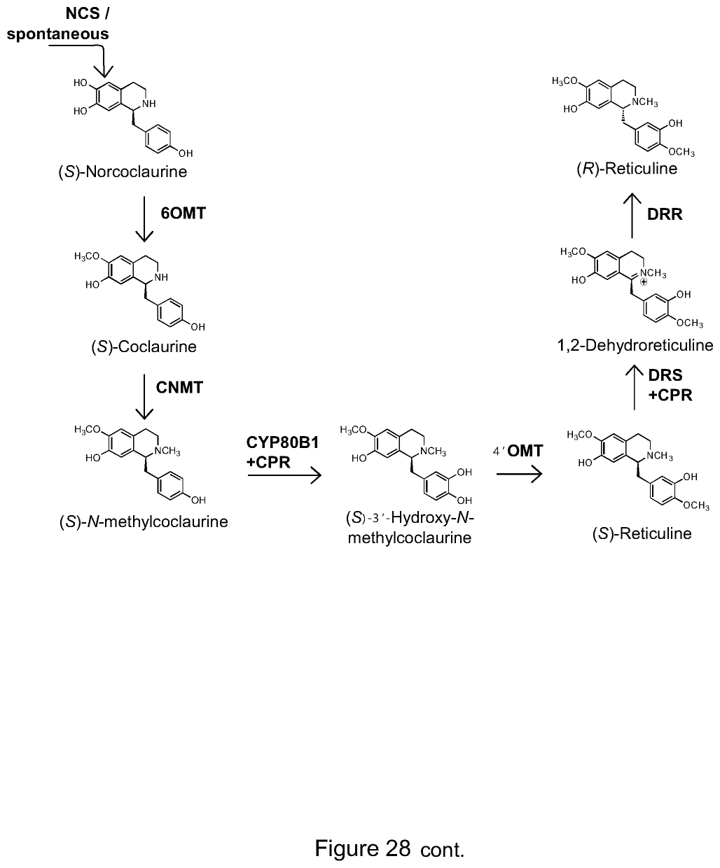

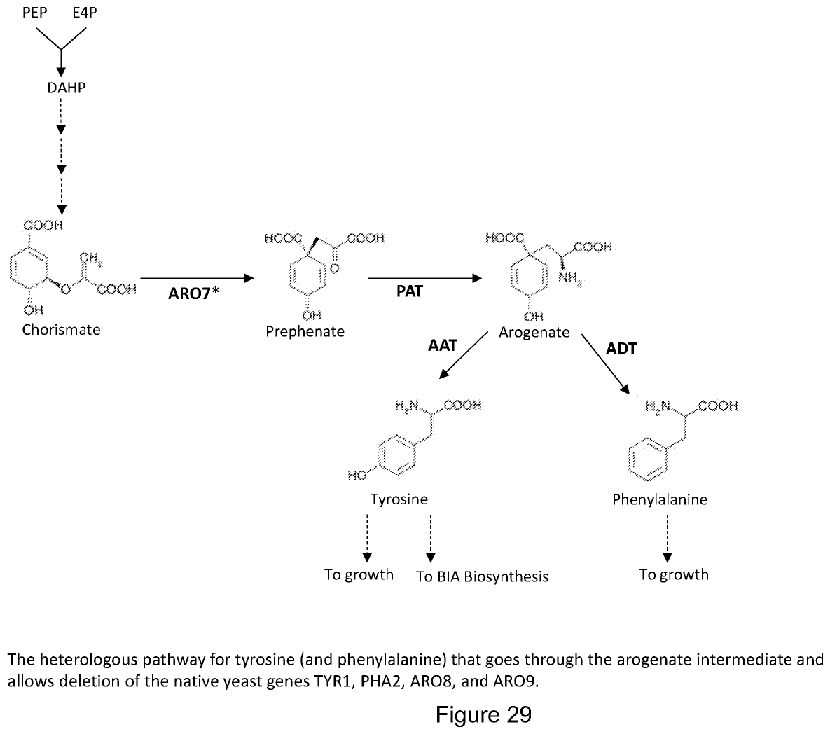

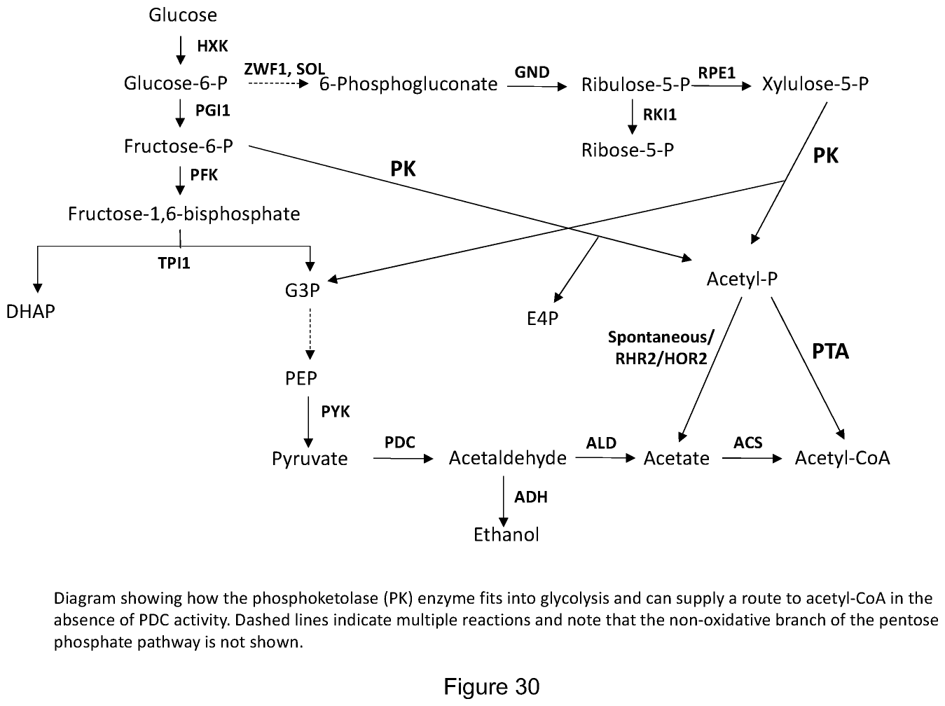

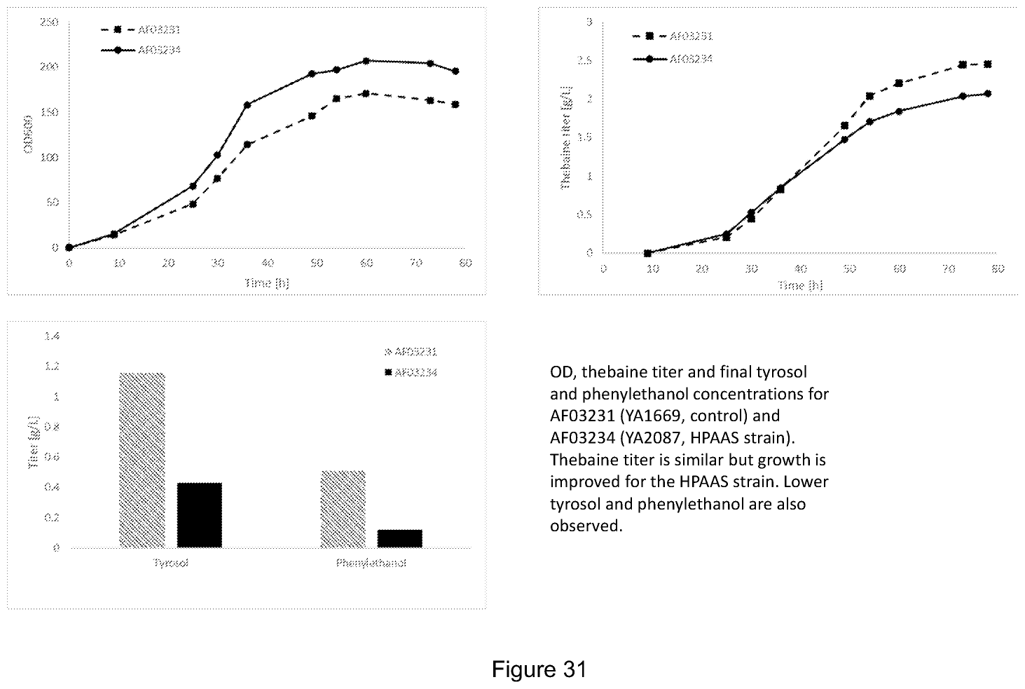

Examples of the genes that are the object of modifications so as to produce BIAs of interest and/or enzymes of interest are discussed below. Additionally, the genes are discussed in the context of a series of Figures that illustrate pathways that are used in generating BIAs of interest and/or enzymes of interest.

[TKL1] In some examples, the engineered host cell may modify the expression of the enzyme transketolase. Transketolase is encoded by the TKL1 gene. In some examples, transketolase catalyzes the reaction of fructose-6-phosphate+glyceraldehyde-3-phosphate ↔xylulose-5-phosphate+erythrose-4-phosphate, as referenced in FIG. 1. An engineered host cell may be modified to include constitutive overexpression of the TKL1 gene in the engineered host cell. Additionally or alternatively, the engineered host cell may be modified to synthetically regulate the expression of the TKL1 gene in the engineered host cell. In some examples, the engineered host cell may be modified to incorporate a copy, copies, or additional copies, of the TKL1 gene. Additionally or alternatively, the engineered host cell may be modified to incorporate the introduction of a strong promoter element for the overexpression of the TKL1 gene within the engineered host cell. The TKL1 gene may be derived from Saccharomyces cerevisiae or another species.

[ZWF1] In some examples, the engineered host cell may modify the expression of the enzyme glucose-6-phosphate dehydrogenase. Glucose-6-phosphate dehydrogenase is encoded by the ZWF1 gene. In some examples, glucose-6-phosphate dehydrogenase catalyzes the reaction of glucose-6-phosphate→6-phosphogluconolactone, as referenced in FIG. 1. An engineered host cell may be modified to delete the coding region of the ZWF1 gene in the engineered host cell. Alternatively, the engineered host cell may be modified to disable the functionality of the ZWF1 gene, such as by introducing an inactivating mutation.

[ARO4] In some examples, the engineered host cell may modify the expression of the enzyme 3-deoxy-D-arabino-heptulosonate-7-phosphate (DAHP) synthase. DAHP synthase is encoded by the ARO4 gene. In some examples, DAHP synthase catalyzes the reaction of erythrose-4-phosphate+phosphoenolpyruvic acid→DAHP, as referenced in FIG. 1. An engineered host cell may modify the ARO4 gene to incorporate one or more feedback inhibition alleviating mutations. In particular, a feedback inhibition alleviating mutation (e.g., ARO4^{IR}) may be incorporated as a directed mutation to a native ARO4 gene at the original locus; as an additional copy introduced as a genetic integration at a separate locus; or as an additional copy on an episomal vector such as a 2-μm or centromeric plasmid. The identifier “FBR” in the mutation ARO4F^{BR} refers to feedback resistant mutants and mutations. The feedback inhibited copy of the DAHP synthase enzyme may be under a native yeast transcriptional regulation, such as when the engineered host cell is a yeast cell. Alternatively, the feedback inhibited copy of the DAHP synthase enzyme may be introduced to the engineered host cell with engineered constitutive or dynamic regulation of protein expression by placing it under the control of a synthetic promoter. In some cases, the ARO4 gene may be derived from Saccharomyces cerevisiae. Examples of modifications to the ARO4 gene include a feedback inhibition resistant mutation, K229L, or Q166K.

[ARO7] In some examples, the engineered host cell may modify the expression of the enzyme chorismate mutase. Chorismate mutase is encoded by the ARO7 gene. In some examples, chorismate mutase catalyzes the reaction of chorismate→prephenate, as referenced in FIG. 1. An engineered host cell may modify the ARO7 gene to incorporate one or more feedback inhibition alleviating mutations. In particular, a feedback inhibition alleviating mutation (e.g., ARO7^{FBR}) may be incorporated as a directed mutation to a native ARO7 gene at the original locus; as an additional copy introduced as a genetic integration at a separate locus; or as an additional copy on an episomal vector such as a 2-μm or centromeric plasmid. The identifier “FBR” in the mutation ARO7^{IR} refers to feedback resistant mutants and mutations. The feedback inhibited copy of the chorismate mutase enzyme may be under a native yeast transcriptional regulation, such as when the engineered host cell is a yeast cell. Alternatively, the feedback inhibited copy of the chorismate mutase enzyme may be introduced to the engineered host cell with engineered constitutive or dynamic regulation of protein expression by placing it under the control of a synthetic promoter. In some cases, the ARO7 gene may be derived from Saccharomyces cerevisiae. Examples of modifications to the ARO7 gene include a feedback inhibition resistant mutation or T226I.

[ARO10] In some examples, the engineered host cell may modify the expression of the enzyme phenylpyruvate decarboxylase. Phenylpyruvate decarboxylase is encoded by the ARO10 gene. In some examples, phenylpyruvate decarboxylase catalyzes the reaction of hydroxyphenylpyruvate→4-hydroxyphenylacetate (4-HPAA), as referenced in FIG. 1. An engineered host cell may be modified to include constitutive overexpression of the ARO10 gene in the engineered host cell. Additionally or alternatively, the engineered host cell may be modified to synthetically regulate the expression of the ARO10 gene in the engineered host cell. In some examples, the engineered host cell may be modified to incorporate a copy, copies, or additional copies, of the ARO10 gene. Additionally or alternatively, the engineered host cell may be modified to incorporate the introduction of a strong promoter element for the overexpression of the ARO10 gene within the engineered host cell. The ARO10 gene may be derived from Saccharomyces cerevisiae or another species.

[ADH2-7, SFA1] In some examples, the engineered host cell may modify the expression of alcohol dehydrogenase enzymes. Alcohol dehydrogenase enzymes may be encoded by one or more of the ADH2, ADH3, ADH4, ADH5, ADH6, ADH7, and SFA1 genes. In some examples, alcohol dehydrogenase catalyzes the reaction of 4-HPAA→tyrosol. An engineered host cell may be modified to delete the coding region of one or more of the ADH2, ADH3, ADH4, ADH5, ADH6, ADH7, and SFA1 genes in the engineered host cell. Alternatively, the engineered host cell may be modified to disable the functionality of one or more of the ADH2, ADH3, ADH4, ADH5, ADH6, ADH7, and SFA1 genes, such as by introducing an inactivating mutation. Alternatively, the engineered host cell may be modified to express the SFA1 gene from a constitutive promoter. In some examples, SFA1 oxidizes S-hydroxymethyglutathione to form S-formylglutathione during glutathione-dependent formaldehyde detoxification.

[ALD2-6] In some examples, the engineered host cell may modify the expression of aldehyde oxidase enzymes. Aldehyde oxidase enzymes may be encoded by one or more of the ALD2, ALD3, ALD4, ALD5, and ALD6 genes. In some examples, aldehyde oxidase catalyzes the reaction of 4-HPAA→hydroxyphenylacetic acid. An engineered host cell may be modified to delete the coding region of one or more of the ALD2, ALD3, ALD4, ALD5, and ALD6 genes in the engineered host cell. Alternatively, the engineered host cell may be modified to disable the functionality of one or more of the ALD2, ALD3, ALD4, ALD5, and ALD6 genes, such as by introducing an inactivating mutation.

[AAD4], [AAD6], [AAD10]], [AAD14], [AAD15], [AAD16] In some examples, the engineered host cell may modify the expression of aryl-alcohol dehydrogenase enzymes. Aryl-alcohol dehydrogenase enzymes may be encoded by one or more of AAD4, AAD6, AAD10, AAD14, AAD15, and AAD16 genes. In some examples, aryl-alcohol dehydrogenase catalyzes the reaction of aromatic aldehyde+NAD⁺→aromatic alcohol+NADH.

[ARI1] In some examples, the engineered host cell may modify the expression of an aldehyde reductase. The aldehyde reductase enzyme may be encoded by the ARI1 gene. In some examples, aldehyde reductase catalyzes the reduction of aromatic aldehyde substrates. In some examples, aldehyde reductase catalyzes the reduction of alophatic aldehyde substrates. In some examples the substrate of the aldehyde reductase ARI1 is 4-hydroxyphenylacetaldehyde (4-HPAA). An engineered host cell may be modified to delete the coding region of ARI. Alternatively, the engineered host cell may be modified to functionally disable ARI1, such as by introducing an inactivating mutation.

[OPI] In some examples, the engineered host cell may modify the expression of a transcriptional regulator of phospholipid biosynthetic genes. The transcriptional regulator may be encoded by the OPI1 gene. In some examples, the transcriptional regulator represses phospholipid biosynthetic genes. An engineered host cell may be modified to delete the coding region of OPI1. Alternatively, the engineered host cell may be modified to functionally disable OPI1, such as by introducing an inactivating mutation.

[ARO9] In some examples, the engineered host cell may modify the expression of the enzyme aromatic aminotransferase. Aromatic aminotransferase is encoded by the ARO9 gene. In some examples, aromatic aminotransferase catalyzes the reaction of hydroxyphenylpyruvate+L-alanine↔tyrosine+pyruvate, as referenced in FIG. 1. An engineered host cell may be modified to include constitutive overexpression of the ARO9 gene in the engineered host cell. Additionally or alternatively, the engineered host cell may be modified to synthetically regulate the expression of the ARO9 gene in the engineered host cell. In some examples, the engineered host cell may be modified to incorporate a copy, copies, or additional copies, of the ARO9 gene. Additionally or alternatively, the engineered host cell may be modified to incorporate the introduction of a strong promoter element for the overexpression of the ARO9 gene within the engineered host cell. The ARO9 gene may be derived from Saccharomyces cerevisiae or another species.

[ARO8] In some examples, the engineered host cell may modify the expression of the enzyme aromatic aminotransferase. Aromatic aminotransferase is encoded by the ARO8 gene. In some examples, aromatic aminotransferase catalyzes the reaction of hydroxyphenylpyruvate+glutamate↔tyrosine+alpha-ketogluterate, as referenced in FIG. 1. An engineered host cell may be modified to include constitutive overexpression of the ARO8 gene in the engineered host cell. Additionally or alternatively, the engineered host cell may be modified to synthetically regulate the expression of the ARO8 gene in the engineered host cell. In some examples, the engineered host cell may be modified to incorporate a copy, copies, or additional copies, of the ARO8 gene. Additionally or alternatively, the engineered host cell may be modified to incorporate the introduction of a strong promoter element for the overexpression of the ARO8 gene within the engineered host cell. The ARO8 gene may be derived from Saccharomyces cerevisiae or another species.

[TYR1] In some examples, the engineered host cell may modify the expression of the enzyme prephenate dehydrogenase. Prephenate dehydrogenase is encoded by the TYR1 gene. In some examples, prephenate dehydrogenase catalyzes the reaction of prephenate+NADP⁺→4-hydroxyphenylpyruvate+CO₂+NADPH, as referenced in FIG. 1. An engineered host cell may be modified to include constitutive overexpression of the TYR1 gene in the engineered host cell. Additionally or alternatively, the engineered host cell may be modified to synthetically regulate the expression of the TYR1 gene in the engineered host cell. In some examples, the engineered host cell may be modified to incorporate a copy, copies, or additional copies, of the TYR1 gene. Additionally or alternatively, the engineered host cell may be modified to incorporate the introduction of a strong promoter element for the overexpression of the TYR1 gene within the engineered host cell. The TYR1 gene may be derived from Saccharomyces cerevisiae or another species.

[HPAAS] In some examples, the engineered host cell may modify the expression of the enzyme 4-hydroxyphenylacetaldehyde synthase. 4-Hydroxyphenylacetaldehyde synthase is encoded by the 4HPAAS gene. In some examples, 4-hydroxyphenylacetaldehyde synthase catalyzes the reaction of L-tyrosine→4-hydroxyphenylacetaldehyde as referenced in FIG. 28. The engineered host cell may be modified to include constitutive expression of the 4HPAAS gene in the engineered host cell. Additionally or alternatively, the engineered host cell may be modified to synthetically regulate the expression of the 4HPAAS gene in the engineered host cell. In some examples, the engineered host cell may be modified to incorporate a copy, copies, or additional copies, of the 4HPAAS gene. Additionally or alternatively, the engineered host cell may be modified to incorporate the introduction of a strong promoter element for the overexpression of the 4HPAAS gene within the engineered host cell. In some cases, the 4HPAAS gene may be codon optimized for expression in Saccharomyces cerevisiae. The 4HPAAS gene may be derived from Petroselinum crispum, Rhodiola rosea, or another species.

[SAH] In some examples, the engineered host cell may modify the expression of the enzyme S-adenosyl-L-homocysteine hydrolase. S-adenosyl-L-homocysteine hydrolase is encoded by the SAH1 gene. In some examples, S-adenosyl-L-homocysteine catalyzes the reaction of S-adenosyl-L-homocysteine→L-homocysteine+adenosine as referenced in FIG. 34. The engineered host cell may be modified to include constitutive expression of the SAH1 gene in the engineered host cell. Additionally or alternatively, the engineered host cell may be modified to synthetically regulate the expression of the SAH1 gene in the engineered host cell. In some examples, the engineered host cell may be modified to incorporate a copy, copies, or additional copies, of the SAH1 gene. Additionally or alternatively, the engineered host cell may be modified to incorporate the introduction of a strong promoter element for the overexpression of the SAH1 gene within the engineered host cell. In some cases, the SAH1 gene may be codon optimized for expression in Saccharomyces cerevisiae. The SAH1 gene may be derived from Saccharomyces cerevisiae or another species.

[SAM] In some examples, the engineered host cell may modify the expression of the enzyme S-adenosylmethionine synthetase. S-adenosylmethionine synthetase is encoded by the SAM1 and SAM2 genes. In some examples, S-adenosylmethionine synthetase catalyzes the reaction of ATP+methionine→S-adenosylmethionine as referenced in FIG. 34. The engineered host cell may be modified to include constitutive expression of the SAM1 or SAM2 gene in the engineered host cell. Additionally or alternatively, the engineered host cell may be modified to synthetically regulate the expression of the SAM1 or SAM2 gene in the engineered host cell. In some examples, the engineered host cell may be modified to incorporate a copy, copies, or additional copies, of the SAM1 or SAM2 gene. Additionally or alternatively, the engineered host cell may be modified to incorporate the introduction of a strong promoter element for the overexpression of the SAM1 or SAM2 gene within the engineered host cell. In some cases, the SAM1 or SAM2 gene may be codon optimized for expression in Saccharomyces cerevisiae. The SAM1 or SAM2 gene may be derived from Saccharomyces cerevisiae or another species.

[PAT] In some examples, the engineered host cell may modify the expression of the enzyme prephenate aminotransferase. Prephenate aminotransferase is encoded by the PAT gene. In some examples, prephenate aminotransferase catalyzes the reaction of prephenate 4 arogenate as referenced in FIG. 29. The engineered host cell may be modified to include constitutive expression of the PAT gene in the engineered host cell. Additionally or alternatively, the engineered host cell may be modified to synthetically regulate the expression of the PAT gene in the engineered host cell. In some examples, the engineered host cell may be modified to incorporate a copy, copies, or additional copies, of the PAT gene. Additionally or alternatively, the engineered host cell may be modified to incorporate the introduction of a strong promoter element for the overexpression of the PAT gene within the engineered host cell. In some cases, the PAT gene may be codon optimized for expression in Saccharomyces cerevisiae. The PAT gene may be derived from Arabidopsis thaliana or another species.

[AAT] In some examples, the engineered host cell may modify the expression of the enzyme arogenate dehydrogenase. Arogenate dehydrogenase is encoded by the AAT gene. In some examples, arogenate dehydrogenase catalyzes the reaction of arogenate 4 tyrosine as referenced in FIG. 29. The engineered host cell may be modified to include constitutive expression of the AAT gene in the engineered host cell. Additionally or alternatively, the engineered host cell may be modified to synthetically regulate the expression of the AAT gene in the engineered host cell. In some examples, the engineered host cell may be modified to incorporate a copy, copies, or additional copies, of the AAT gene. Additionally or alternatively, the engineered host cell may be modified to incorporate the introduction of a strong promoter element for the overexpression of the AAT gene within the engineered host cell. In some cases, the AAT gene may be codon optimized for expression in Saccharomyces cerevisiae. The AAT gene may be derived from Arabidopsis thaliana or another species.

[ADT] In some examples, the engineered host cell may modify the expression of the enzyme arogenate dehydrogenase. Arogenate dehydrogenase is encoded by the ADT gene. In some examples, arogenate dehydrogenase catalyzes the reaction of arogenate→phenylalanine as referenced in FIG. 29. The engineered host cell may be modified to include constitutive expression of the ADT gene in the engineered host cell. Additionally or alternatively, the engineered host cell may be modified to synthetically regulate the expression of the ADT gene in the engineered host cell. In some examples, the engineered host cell may be modified to incorporate a copy, copies, or additional copies, of the ADT gene. Additionally or alternatively, the engineered host cell may be modified to incorporate the introduction of a strong promoter element for the overexpression of the ADT gene within the engineered host cell. In some cases, the ADT gene may be codon optimized for expression in Saccharomyces cerevisiae. The ADT gene may be derived from Papaver somniferum, Arabidopsis thaliana or another species.

[PK] In some examples, the engineered host cell may modify the expression of the enzyme phosphoketolase. Phosphoketolase is encoded by the PK gene. In some examples, phosphoketolase catalyzes the reaction of fructose-6-phosphate→erythrose-4-phosphate+acetyl-phosphate as referenced in FIG. 30. In some examples, phosphoketolase catalyzes the reaction of xylulose-5-phosphate→glyceraldehyde-3-phosphate+acetyl-phosphate as referenced in FIG. 30. The engineered host cell may be modified to include constitutive expression of the PK gene in the engineered host cell. Additionally or alternatively, the engineered host cell may be modified to synthetically regulate the expression of the PK gene in the engineered host cell. In some examples, the engineered host cell may be modified to incorporate a copy, copies, or additional copies, of the PK gene. Additionally or alternatively, the engineered host cell may be modified to incorporate the introduction of a strong promoter element for the overexpression of the PK gene within the engineered host cell. In some cases, the PK gene may be codon optimized for expression in Saccharomyces cerevisiae. The PK gene may be derived from Bifidobacterium breve, Bifidobacterium animalis, Leuconostoc mesenteroides, Clostridium acetobutylicum, or another species.

[PTA] In some examples, the engineered host cell may modify the expression of the enzyme phosphate acetyltransferase. Phosphate acetyltransferase is encoded by the PTA gene. In some examples, phosphate acetyltransferase catalyzes the reaction of acetyl-CoA+phosphate→acetyl-phosphate+CoA as referenced in FIG. 30. The engineered host cell may be modified to include constitutive expression of the PTA gene in the engineered host cell. Additionally or alternatively, the engineered host cell may be modified to synthetically regulate the expression of the PTA gene in the engineered host cell. In some examples, the engineered host cell may be modified to incorporate a copy, copies, or additional copies, of the PTA gene. Additionally or alternatively, the engineered host cell may be modified to incorporate the introduction of a strong promoter element for the overexpression of the PTA gene within the engineered host cell. In some cases, the PTA gene may be codon optimized for expression in Saccharomyces cerevisiae. The PTA gene may be derived from Escherichia coli, Clostridium kluyveri, Methanosarcina thermophila, Salmonella enterica, Bacillus subtilis or another species.

[UGT] In some examples, the engineered host cell may modify the expression of the enzyme uridine 5′-diphospho-glucosyltransferase. Uridine 5′-diphospho-glucosyltransferase activity is encoded by the UGT gene. In some examples, uridine 5′-diphospho-glucosyltransferase catalyzes the reaction of UDP-glucose+a phenol→UDP+an aryl beta-D-glucoside. The engineered host cell may be modified to include constitutive expression of the UGT gene in the engineered host cell. Additionally or alternatively, the engineered host cell may be modified to synthetically regulate the expression of the UGT gene in the engineered host cell. In some examples, the engineered host cell may be modified to incorporate a copy, copies, or additional copies, of the UGT gene. Additionally or alternatively, the engineered host cell may be modified to incorporate the introduction of a strong promoter element for the overexpression of the UGT gene within the engineered host cell. In some cases, the UGT gene may be codon optimized for expression in Saccharomyces cerevisiae. The UGT gene may be derived from Rhodiola rosea or another species.

[TYR] In some examples, the engineered host cell may modify the expression of the enzyme tyrosinase. Tyrosinase is encoded by the TYR gene. In some examples, tyrosinase catalyzes the reaction of tyrosine→L-DOPA, as referenced in FIGS. 1 and 2. In other examples, tyrosinase catalyzes the reaction of L-DOPA→dopaquinone. An engineered host cell may be modified to include constitutive expression of the TYR gene in the engineered host cell. Additionally or alternatively, the engineered host cell may be modified to synthetically regulate the expression of the TYR gene in the engineered host cell. In some examples, the engineered host cell may be modified to incorporate a copy, copies, or additional copies, of the TYR gene. Additionally or alternatively, the engineered host cell may be modified to incorporate the introduction of a strong promoter element for the overexpression of the TYR gene within the engineered host cell. The TYR gene may be derived from Ralstonia solanacearum, Agaricus bisporus, Escherichia coli or another species.

[TyrH] In some examples, the engineered host cell may modify the expression of the enzyme tyrosine hydroxylase. Tyrosine hydroxylase is encoded by the TyrH gene. In some examples, tyrosine hydroxylase catalyzes the reaction of tyrosine→L-DOPA, as referenced in FIGS. 1 and 2. An engineered host cell may be modified to include constitutive expression of the TyrH gene in the engineered host cell. Additionally or alternatively, the engineered host cell may be modified to synthetically regulate the expression of the TyrH gene in the engineered host cell. In some examples, the engineered host cell may be modified to incorporate a copy, copies, or additional copies, of the TyrH gene. Additionally or alternatively, the engineered host cell may be modified to incorporate the introduction of a strong promoter element for the overexpression of the TyrH gene within the engineered host cell. The TyrH gene may be derived from Homo sapiens, Rattus norvegicus, Mus musculus, Drosophilia melanogaster, Apis melifera, or another species.

[DODC] In some examples, the engineered host cell may modify the expression of the enzyme L-DOPA decarboxylase. L-DOPA decarboxylase is encoded by the DODC gene. In some examples, L-DOPA decarboxylase catalyzes the reaction of L-DOPA 4 dopamine, as referenced in FIG. 1. An engineered host cell may be modified to include constitutive expression of the DODC gene in the engineered host cell. Additionally or alternatively, the engineered host cell may be modified to synthetically regulate the expression of the DODC gene in the engineered host cell. In some examples, the engineered host cell may be modified to incorporate a copy, copies, or additional copies, of the DODC gene. Additionally or alternatively, the engineered host cell may be modified to incorporate the introduction of a strong promoter element for the overexpression of the DODC gene within the engineered host cell. The DODC gene may be derived from Pseudomonas putida, Rattus norvegicus, or another species.

[TYDC] In some examples, the engineered host cell may modify the expression of the enzyme tyrosine/DOPA decarboxylase. Tyrosine/DOPA decarboxylase is encoded by the TYDC gene. In some examples, tyrosine/DOPA decarboxylase catalyzes the reaction of L-DOPA 4 dopamine, as referenced in FIG. 3. An engineered host cell may be modified to include constitutive expression of the TYDC gene in the engineered host cell. Additionally or alternatively, the engineered host cell may be modified to synthetically regulate the expression of the TYDC gene in the engineered host cell. In some examples, the engineered host cell may be modified to incorporate a copy, copies, or additional copies, of the TYDC gene. Additionally or alternatively, the engineered host cell may be modified to incorporate the introduction of a strong promoter element for the overexpression of the TYDC gene within the engineered host cell. The TYDC gene may be derived from Papaver somniferum or another species.

[MAO] In some examples, the engineered host cell may modify the expression of the enzyme monoamine oxidase. Monoamine oxidase is encoded by the MAO gene. In some examples, monoamine oxidase catalyzes the reaction of dopamine→3,4-DHPA, as referenced in FIGS. 1 and 3. An engineered host cell may be modified to include constitutive expression of the MAO gene in the engineered host cell. Additionally or alternatively, the engineered host cell may be modified to synthetically regulate the expression of the MAO gene in the engineered host cell. In some examples, the engineered host cell may be modified to incorporate a copy, copies, or additional copies, of the MAO gene. Additionally or alternatively, the engineered host cell may be modified to incorporate the introduction of a strong promoter element for the overexpression of the MAO gene within the engineered host cell. In some cases, the MAO gene may be codon optimized for expression in Saccharomyces cerevisiae. The MAO gene may be derived from Escherichia coli, Homo sapiens, Micrococcus luteus, or another species.

[NCS] In some examples, the engineered host cell may modify the expression of the enzyme norcoclaurine synthase. Norcoclaurine synthase is encoded by the NCS gene. In some examples, norcoclaurine synthase catalyzes the reaction of 4-HPAA+dopamine→(S)-norcoclaurine, as referenced in FIGS. 1 and 3. In particular, FIG. 1 illustrates a biosynthetic scheme for conversion of L-tyrosine to reticuline via norcoclaurine, in accordance with some embodiments of the disclosure. FIG. 1 provides the use of the enzymes TyrH, tyrosine hydroxylase; DODC, DOPA decarboxylase; NCS, norcoclaurine synthase, as discussed herein; 60MT, 6-O-methyltransferase; CNMT, coclaurine N-methyltransferase; CYP80B1, cytochrome P450 80B1; CPR, cytochrome P450 NADPH reductase; 4′OMT, 3′hydroxy-N-methylcoclaurine 4′-O-methyltransferase. L-DOPA, L-3,4-dihydroxyphenylalanine; and 4-HPAA, 4-hydroxyphenylacetylaldehyde. Of the enzymes that are illustrated in FIG. 1, 4-HPAA and L-tyrosine are naturally synthesized in yeast. All other listed metabolites are not naturally produced in yeast. Additionally, although TyrH may catalyze the conversion of L-tyrosine to L-DOPA, other enzymes may also be used to perform this step as described in the specification. For example, tyrosinases may also be used to perform the conversion of L-tyrosine to L-DOPA. In addition, other enzymes such as cytochrome P450 oxidases may also be used to perform the conversion of L-tyrosine to L-DOPA. Such enzymes may exhibit oxidase activity on related BIA precursor compounds including L-DOPA and L-tyrosine.

Additionally, norcoclaurine synthase catalyzes the reaction of 3,4-DHPAA+dopamine→(S)-norlaudanosoline, as referenced in FIGS. 1 and 3. In particular, FIG. 3 illustrates a biosynthetic scheme for conversion of L-tyrosine to reticuline via norlaudanosoline, in accordance with some embodiments of the disclosure. FIG. 3 provides the use of the enzymes TyrH, tyrosine hydroxylase; DODC, DOPA decarboxylase; maoA, monoamine oxidase; NCS, norcoclaurine synthase; 60MT, 6-O-methyltransferase; CNMT, coclaurine N-methyltransferase; 4′OMT, 3′hydroxy-N-methylcoclaurine 4′-O-methyltransferase. L-DOPA, L-3,4-dihydroxyphenylalanine; and 3,4-DHPAA, 3,4-dihydroxyphenylacetaldehyde. Of the enzymes that are illustrated in FIG. 3, L-tyrosine is naturally synthesized in yeast.

An engineered host cell may be modified to include constitutive expression of the NCS gene in the engineered host cell. Additionally or alternatively, the engineered host cell may be modified to synthetically regulate the expression of the NCS gene in the engineered host cell. In some examples, the engineered host cell may be modified to incorporate a copy, copies, or additional copies, of the NCS gene. Additionally or alternatively, the engineered host cell may be modified to incorporate the introduction of a strong promoter element for the overexpression of the NCS gene within the engineered host cell. Additionally, the norcoclaurine synthase may have an N-terminal truncation. In some cases, the NCS gene may be codon optimized for expression in Saccharomyces cerevisiae. The NCS gene may be derived from Coptis japonica, Papaver somniferum, Papver bracteatum, Thalicitum flavum, Corydalis saxicola, or another species.

[60MT] In some examples, the engineered host cell may modify the expression of the enzyme norcoclaurine 6-O-methyltransferase. Norcoclaurine 6-O-methyltransferase is encoded by the 60MT gene. In some examples, norcoclaurine 6-O-methyltransferase catalyzes the reaction of norcoclaurine 4 coclaurine, as referenced in FIG. 1. In other examples, norcoclaurine 6-O-methyltransferase catalyzes the reaction of norlaudanosoline→3′hydroxycoclaurine, as well as other reactions detailed herein, such as those provided in FIG. 3. Additionally, the engineered host cell may be modified to include constitutive expression of the 60MT gene in the engineered host cell. Additionally or alternatively, the engineered host cell may be modified to synthetically regulate the expression of the 60MT gene in the engineered host cell. In some examples, the engineered host cell may be modified to incorporate a copy, copies, or additional copies, of the 60MT gene. Additionally or alternatively, the engineered host cell may be modified to incorporate the introduction of a strong promoter element for the overexpression of the 60MT gene within the engineered host cell. The 60MT gene may be derived from P. somniferum, T. flavum, Coptis japonica, or another species.

[CNMT] In some examples, the engineered host cell may modify the expression of the enzyme coclaurine-N-methyltransferase. Coclaurine-N-methyltransferase is encoded by the CNMT gene. In some examples, coclaurine-N-methyltransferase catalyzes the reaction of coclaurine→N-methylcoclaurine, as referenced in FIG. 1. In other examples, the coclaurine-N-methyltransferase enzyme may catalyze the reaction of 3′hydroxycoclaurine→3′hydroxy-N-methylcoclaurine. In other examples, coclaurine-N-methyltransferase may catalyze other reactions detailed herein, such as those provided in FIG. 3. Additionally, the engineered host cell may be modified to include constitutive expression of the CNMT gene in the engineered host cell. Additionally or alternatively, the engineered host cell may be modified to synthetically regulate the expression of the CNMT gene in the engineered host cell. In some examples, the engineered host cell may be modified to incorporate a copy, copies, or additional copies, of the CNMT gene. Additionally or alternatively, the engineered host cell may be modified to incorporate the introduction of a strong promoter element for the overexpression of the CNMT gene within the engineered host cell. The CNMT gene may be derived from P. somniferum, T. flavum, Coptis japonica, or another species.

[4′OMT] In some examples, the engineered host cell may modify the expression of the enzyme 4′-O-methyltransferase. 4′-O-methyltransferase is encoded by the 4′OMT gene. In some examples, 4′-O-methyltransferase catalyzes the reaction of 3′-hydroxy-N-methylcoclaurine 4 reticuline, as referenced in FIG. 1. In other examples, 4′-O-methyltransferase catalyzes other reactions detailed herein, such as those provided in FIG. 3. Additionally, the engineered host cell may be modified to include constitutive expression of the 4′OMT gene in the engineered host cell. Additionally or alternatively, the engineered host cell may be modified to synthetically regulate the expression of the 4′OMT gene in the engineered host cell. In some examples, the engineered host cell may be modified to incorporate a copy, copies, or additional copies, of the 4′OMT gene. Additionally or alternatively, the engineered host cell may be modified to incorporate the introduction of a strong promoter element for the overexpression of the 4′OMT gene within the engineered host cell. The 4′OMT gene may be derived from P. somniferum, T. flavum, Coptis japonica, or another species.

[CYP80B1] In some examples, the engineered host cell may modify the expression of the enzyme cytochrome P450 80B1. Cytochrome P450 80B1 is encoded by the CYP80B1 gene. In some examples, cytochrome P450 80B1 catalyzes the reaction of N-methylcoclaurine+3′-hydroxy-N-methylcoclaurine, as referenced in FIG. 1. An engineered host cell may be modified to include constitutive expression of the cytochrome P450 80B1 gene in the engineered host cell. Additionally or alternatively, the engineered host cell may be modified to synthetically regulate the expression of the cytochrome P450 80B1 gene in the engineered host cell. In some examples, the engineered host cell may be modified to incorporate a copy, copies, or additional copies, of the cytochrome P450 80B1 gene. Additionally or alternatively, the engineered host cell may be modified to incorporate the introduction of a strong promoter element for the overexpression of the cytochrome P450 80B1 gene within the engineered host cell. In some cases, the CYP80B1 gene may be codon optimized for expression in Saccharomyces cerevisiae. The cytochrome P450 80B1 gene may be derived from P. somniferum, E. californica, T. flavum, or another species.

[FOL2] In some examples, the engineered host cell may modify the expression of the enzyme GTP cyclohydrolase. GTP cyclohydrolase is encoded by the FOL2 gene. In some examples, GTP cyclohydrolase catalyzes the reaction of GTP 4 dihydroneopterin triphosphate, as referenced in FIG. 2. The engineered host cell may be modified to include constitutive overexpression of the FOL2 gene in the engineered host cell. The engineered host cell may also be modified to include native regulation. Additionally or alternatively, the engineered host cell may be modified to synthetically regulate the expression of the FOL2 gene in the engineered host cell. In some examples, the engineered host cell may be modified to incorporate a copy, copies, or additional copies, of the FOL2 gene. Additionally or alternatively, the engineered host cell may be modified to incorporate the introduction of a strong promoter element for the overexpression of the FOL2 gene within the engineered host cell. The FOL2 gene may be derived from Saccharomyces cerevisiae, Homo sapiens, Mus musculus, or another species.

[PTPS] In some examples, the engineered host cell may modify the expression of the enzyme 6-pyruvoyl tetrahydrobiopterin (PTP) synthase. Pyruvoyl tetrahydrobiopterin synthase is encoded by the PTPS gene. In some examples, 6-pyruvoyl tetrahydrobiopterin synthase catalyzes the reaction of dihydroneopterin triphosphate→PTP, as referenced in FIG. 2. The engineered host cell may be modified to include constitutive expression of the PTPS gene in the engineered host cell. Additionally or alternatively, the engineered host cell may be modified to synthetically regulate the expression of the PTPS gene in the engineered host cell. In some examples, the engineered host cell may be modified to incorporate a copy, copies, or additional copies, of the PTPS gene. Additionally or alternatively, the engineered host cell may be modified to incorporate the introduction of a strong promoter element for the overexpression of the PTPS gene within the engineered host cell. In some cases, the PTPS gene may be codon optimized for expression in Saccharomyces cerevisiae. The PTPS gene may be derived from Rattus norvegicus, Homo sapiens, Mus musculus, or another species.

[SepR] In some examples, the engineered host cell may modify the expression of the enzyme sepiapterin reductase. Sepiapterin reductase is encoded by the SepR gene. In some examples, sepiapterin reductase catalyzes the reaction of PTP→BH₄, as referenced in FIG. 2. The engineered host cell may be modified to include constitutive expression of the SepR gene in the engineered host cell. Additionally or alternatively, the engineered host cell may be modified to synthetically regulate the expression of the SepR gene in the engineered host cell. In some examples, the engineered host cell may be modified to incorporate a copy, copies, or additional copies, of the SepR gene. Additionally or alternatively, the engineered host cell may be modified to incorporate the introduction of a strong promoter element for the overexpression of the SepR gene within the engineered host cell. In some cases, the SepR gene may be codon optimized for expression in Saccharomyces cerevisiae. The SepR gene may be derived from Rattus norvegicus, Homo sapiens, Mus musculus, or another species.

[PCD] In some examples, the engineered host cell may modify the expression of the enzyme 4a-hydroxytetrahydrobiopterin (pterin-4a-carbinolamine) dehydratase. 4a-hydroxytetrahydrobiopterin dehydratase is encoded by the PCD gene. In some examples, 4a-hydroxytetrahydrobiopterin dehydratase catalyzes the reaction of 4a-hydroxytetrahydrobiopterin→H₂O+quinonoid dihydropteridine, as referenced in FIG. 2. The engineered host cell may be modified to include constitutive expression of the PCD gene in the engineered host cell. Additionally or alternatively, the engineered host cell may be modified to synthetically regulate the expression of the PCD gene in the engineered host cell. In some examples, the engineered host cell may be modified to incorporate a copy, copies, or additional copies, of the PCD gene. Additionally or alternatively, the engineered host cell may be modified to incorporate the introduction of a strong promoter element for the overexpression of the PCD gene within the engineered host cell. In some cases, the PCD gene may be codon optimized for expression in Saccharomyces cerevisiae. The PCD gene may be derived from Rattus norvegicus, Homo sapiens, Mus musculus, or another species.

[QDHPR] In some examples, the engineered host cell may modify the expression of the enzyme quinonoid dihydropteridine reductase. Quinonoid dihydropteridine reductase is encoded by the QDHPR gene. In some examples, quinonoid dihydropteridine reductase catalyzes the reaction of quinonoid dihydropteridine→BH₄, as referenced in FIG. 2. The engineered host cell may be modified to include constitutive expression of the QDHPR gene in the engineered host cell. Additionally or alternatively, the engineered host cell may be modified to synthetically regulate the expression of the QDHPR gene in the engineered host cell. In some examples, the engineered host cell may be modified to incorporate a copy, copies, or additional copies, of the QDHPR gene. Additionally or alternatively, the engineered host cell may be modified to incorporate the introduction of a strong promoter element for the overexpression of the QDHPR gene within the engineered host cell. In some cases, the QDHPR gene may be codon optimized for expression in Saccharomyces cerevisiae. The QDHPR gene may be derived from Rattus norvegicus, Homo sapiens, Mus musculus, or another species.

[DHFR] In some examples, the engineered host cell may modify the expression of the enzyme dihydrofolate reductase. Dihydrofolate reductase is encoded by the DHFR gene. In some examples, dihydrofolate reductase catalyzes the reaction of 7,8-dihydrobiopterin (BH₂)→5,6,7,8-tetrahydrobiopterin (BH₄), as referenced in FIG. 2. This reaction may be useful in recovering BH₄ as a co-substrate for the conversation of tyrosine to L-DOPA, as illustrated in FIG. 2. The engineered host cell may be modified to include constitutive expression of the DHFR gene in the engineered host cell. Additionally or alternatively, the engineered host cell may be modified to synthetically regulate the expression of the DHFR gene in the engineered host cell. In some examples, the engineered host cell may be modified to incorporate a copy, copies, or additional copies, of the DHFR gene. Additionally or alternatively, the engineered host cell may be modified to incorporate the introduction of a strong promoter element for the overexpression of the DHFR gene within the engineered host cell. In some cases, the DHFR gene may be codon optimized for expression in Saccharomyces cerevisiae. The DHFR gene may be derived from Rattus norvegicus, Homo sapiens, or another species.

[DRS-DRR] As discussed above with regard to epimerizing 1-BIAs, the engineered host cell may modify the expression of a BIA epimerase. The BIA epimerase is encoded by the DRS-DRR gene. In some examples, DRS-DRR may also be referred to as CYP—COR. In some examples, an engineered split version, or an engineered fused version, of a BIA epimerase catalyzes the conversion of (S)-1-BIA→(R)-1-BIA, as referenced in FIG. 4. In particular, FIG. 4 illustrates a biosynthetic scheme for conversion of L-tyrosine to morphinan alkaloids, in accordance with some embodiments of the invention. FIG. 4 provides the use of the enzymes CPR, cytochrome P450 reductase; DRS-DRR, dehydroreticuline synthase and dehydroreticuline reductase; SalSyn, salutaridine synthase; SalR, salutaridine reductase; SalAT, salutaridinol 7-O-acetyltransferase; T6ODM, thebaine 6-O-demethylase; COR, codeinone reductase; and CODM, codeine-O-demethylase.

The engineered host cell may be modified to include constitutive expression of the engineered DRS-DRR gene in the engineered host cell. In some cases, the engineered DRS-DRR gene may encode an engineered fusion epimerase. In some cases, the engineered DRS-DRR gene may encode an engineered split epimerase. Additionally or alternatively, the engineered host cell may be modified to synthetically regulate the expression of the DRS-DRR gene in the engineered host cell. In some examples, the engineered host cell may be modified to incorporate a copy, copies, or additional copies, of the DRS-DRR gene. Additionally or alternatively, the engineered host cell may be modified to incorporate the introduction of a strong promoter element for the overexpression of the DRS-DRR gene within the engineered host cell. The DRS-DRR gene may be derived from Papaver bracteatum, Papaver somniferum, Papaver setigerum, Chelidonium majus, or another species.

[CPR] In some examples, the engineered host cell may modify the expression of the enzyme cytochrome P450 reductase. The cytochrome P450 reductase is encoded by the CPR gene. In some examples, the cytochrome P450 reductase catalyzes the reaction of (R)-reticuline→salutaridine, as referenced in FIG. 4. Additionally, the cytochrome P450 reductase catalyzes other reactions such as those described in FIGs. throughout the application. In some embodiments, the CPR catalyzes a 14-hydroxylation reaction. In some embodiments, the CPR catelizes a 14-hydroxylation reaction with a P450. In some embodiments, the 14-hydroxylation reaction comprises 14-hydroxylation of a morphanin alkaloid. The engineered host cell may be modified to include constitutive expression of the CPR gene in the engineered host cell. Additionally or alternatively, the engineered host cell may be modified to synthetically regulate the expression of the CPR gene in the engineered host cell. In some examples, the engineered host cell may be modified to incorporate a copy, copies, or additional copies, of the CPR gene. Additionally or alternatively, the engineered host cell may be modified to incorporate the introduction of a strong promoter element for the overexpression of the CPR gene within the engineered host cell. In certain embodiments, the CPR gene may be derived from E. californica, P. somniferum, H. sapiens, S. cerevisiae, A. thaliana, or another species.

[SalSyn] In some examples, the engineered host cell may modify the expression of the enzyme salutaridine synthase. The salutaridine synthase is encoded by the SalSyn gene. In some examples, the salutaridine synthase catalyzes the reaction of (R)-reticuline 4 salutaridine, as referenced in FIG. 4. The engineered host cell may be modified to include constitutive expression of the SalSyn gene in the engineered host cell. Additionally or alternatively, the engineered host cell may be modified to synthetically regulate the expression of the SalSyn gene in the engineered host cell. In some examples, the engineered host cell may be modified to incorporate a copy, copies, or additional copies, of the SalSyn gene. Additionally or alternatively, the engineered host cell may be modified to incorporate the introduction of a strong promoter element for the overexpression of the SalSyn gene within the engineered host cell. In some cases, the SalSyn gene may be codon optimized for expression in Saccharomyces cerevisiae. In some examples the SalSyn may be modified at the N-terminus. The SalSyn gene may be derived from Papaver somniferum, Papaver spp, Chelidonium majus, or another species.

[SalR] In some examples, the engineered host cell may modify the expression of the enzyme salutaridine reductase. Salutaridine reductase is encoded by the SalR gene. In some examples, salutaridine reductase reversibly catalyzes the reaction of salutaridinol 4 salutaridine, as referenced in FIG. 4. The engineered host cell may be modified to include constitutive expression of the SalR gene in the engineered host cell. Additionally or alternatively, the engineered host cell may be modified to synthetically regulate the expression of the SalR gene in the engineered host cell. In some examples, the engineered host cell may be modified to incorporate a copy, copies, or additional copies, of the SalR gene. Additionally or alternatively, the engineered host cell may be modified to incorporate the introduction of a strong promoter element for the overexpression of the SalR gene within the engineered host cell. In some cases, the SalR gene may be codon optimized for expression in Saccharomyces cerevisiae. The SalR gene may be derived from Papaver somniferum, Papaver bracteatum, Papaver spp., Chelidonium majus, or another species.

[SalAT] In some examples, the engineered host cell may modify the expression of the enzyme acetyl-CoA:salutaridinol 7-O-acetyltransferase. Acetyl-CoA:salutaridinol 7-O-acetyltransferase is encoded by the SalAT gene. In some examples, acetyl-CoA:salutaridinol 7-O-acetyltransferase catalyzes the reaction of acetyl-CoA+salutaridinol→CoA+7-O-acetylsalutaridinol, as referenced in FIG. 4. The engineered host cell may be modified to include constitutive expression of the SalAT gene in the engineered host cell. Additionally or alternatively, the engineered host cell may be modified to synthetically regulate the expression of the SalAT gene in the engineered host cell. In some examples, the engineered host cell may be modified to incorporate a copy, copies, or additional copies, of the SalAT gene. Additionally or alternatively, the engineered host cell may be modified to incorporate the introduction of a strong promoter element for the overexpression of the SalAT gene within the engineered host cell. In some cases, the SalAT gene may be codon optimized for expression in Saccharomyces cerevisiae. The SalAT gene may be derived from Papaver somniferum, Papaver bracteatum, Papaver orientale, Papaver spp., or another species.

[TS] In some examples, the engineered host cell may modify the expression of the enzyme thebaine synthase. Thebaine synthase is encoded by the TS gene. In some examples, a thebaine synthase or an engineered thebaine synthase catalyzes the reaction of 7-O-acetylsalutaridinol→thebaine+acetate, as referenced in FIG. 4. In some examples, the reaction of 7-O-acetylsalutaridinol→thebaine+acetate occurs spontaneously, but thebaine synthase catalyzes some portion of this reaction. In particular, FIG. 4 illustrates a biosynthetic scheme for conversion of L-tyrosine to morphinan alkaloids, in accordance with some embodiments of the disclosure. FIG. 4 provides the use of the enzymes CPR, cytochrome P450 reductase; DRS-DRR, dehydroreticuline synthase and dehydroreticuline reductase; SalSyn, salutaridine synthase; SalR, salutaridine reductase; SalAT, salutaridinol 7-O-acetyltransferase; TS, thebaine synthase; T6ODM, thebaine 6-O-demethylase; COR, codeinone reductase; and CODM, codeine-O-demethylase.

The engineered host cell may be modified to include constitutive expression of the TS gene or the engineered TS gene in the engineered host cell. In some cases, the engineered TS gene may encode an engineered fusion enzyme. Additionally or alternatively, the engineered host cell may be modified to synthetically regulate the expression of the TS gene in the engineered host cell. In some examples, the engineered host cell may be modified to incorporate a copy, copies, or additional copies, of the TS gene. Additionally or alternatively, the engineered host cell may be modified to incorporate the introduction of a strong promoter element for the overexpression of the TS gene within the engineered host cell. In some cases, the TS gene may be codon optimized for expression in Saccharomyces cerevisiae. The TS gene may be derived from Papaver somniferum, Papaver bracteatum, Papaver orientale, Papaver spp., or another species.

[T6ODM] In some examples, the engineered host cell may modify the expression of the enzyme thebaine 6-O-demethylase. Thebaine 6-O demethylase is encoded by the T6ODM gene. In some examples, thebaine 6-O-demethylase catalyzes the reaction of thebaine→neopinone, as referenced in FIG. 4. Once the neopinone has been produced, the neopinone may be converted to codeinone. The conversion of neopinone→codeinone may occur spontaneously. Alternatively, the conversion of neopinone→codeinone may occur as a result of a catalyzed reaction. In other examples, the T6ODM enzyme may catalyze the O-demethylation of substrates other than thebaine. For example, T6ODM may O-demethylate oripavine to produce morphinone. Alternatively, T6ODM may catalyze the O-demethylation of BIAs within the 1-benzylisoquinoline, protoberberine, or protopine classes such as papaverine, canadine, and allocryptopine, respectively. The engineered host cell may be modified to include constitutive expression of the T6ODM gene in the engineered host cell. Additionally or alternatively, the engineered host cell may be modified to synthetically regulate the expression of the T6ODM gene in the engineered host cell. In some examples, the engineered host cell may be modified to incorporate a copy, copies, or additional copies, of the T6ODM gene. Additionally or alternatively, the engineered host cell may be modified to incorporate the introduction of a strong promoter element for the overexpression of the T6ODM gene within the engineered host cell. In some cases, the T6ODM gene may be codon optimized for expression in Saccharomyces cerevisiae. The T6ODM gene may be derived from Papaver somniferum, or another species.

[NPI] In some examples, the engineered host cell may modify the expression of the enzyme neopinone isomerase. Neopinone isomerase is encoded by the NPI gene. In some examples, a neopinone isomerase or an engineered neopinone isomerase catalyzes the reaction of neopinone 4 codeinone, as referenced in FIG. 4. In some examples, the reaction of neopinone 4 codeinone occurs spontaneously, but neopinone isomerase catalyzes some portion of this reaction. In particular, FIG. 4 illustrates a biosynthetic scheme for conversion of L-tyrosine to morphinan alkaloids, in accordance with some embodiments of the invention. FIG. 4 provides the use of the enzymes CPR, cytochrome P450 reductase; DRS-DRR, dehydroreticuline synthase and dehydroreticuline reductase; SalSyn, salutaridine synthase; SalR, salutaridine reductase; SalAT, salutaridinol 7-O-acetyltransferase; TS, thebaine synthase; T6ODM, thebaine 6-O-demethylase; NPI, neopinone isomerase; COR, codeinone reductase; and CODM, codeine-O-demethylase.

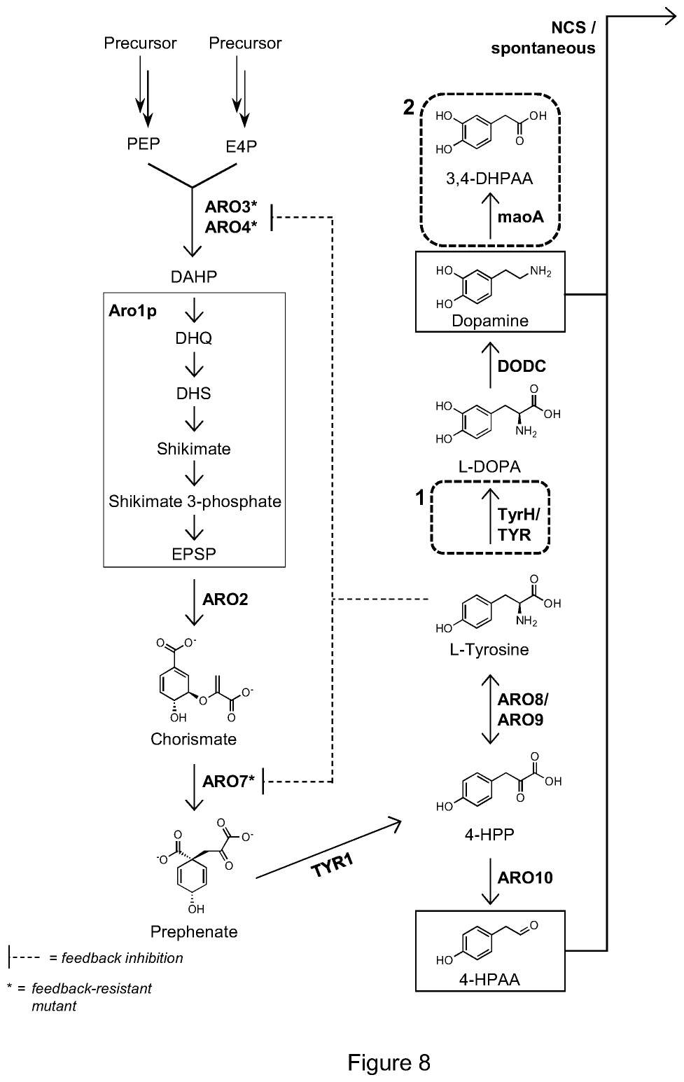

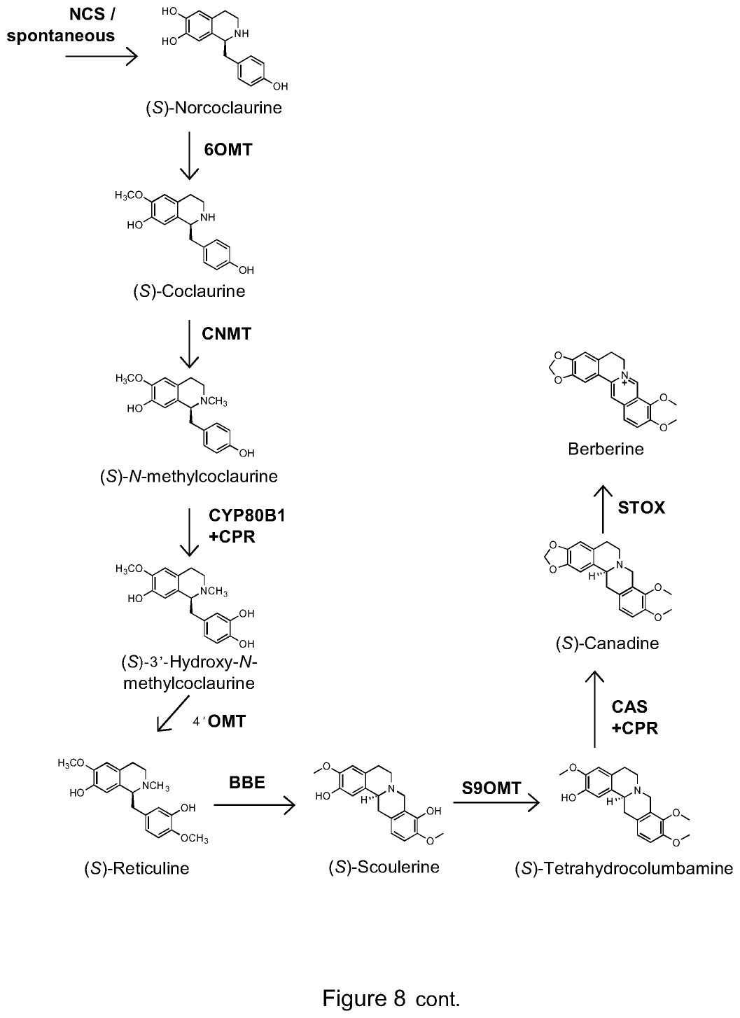

The engineered host cell may be modified to include constitutive expression of the NPI gene or the engineered NPI gene in the engineered host cell. In some cases, the engineered NPI gene may encode an engineered fusion enzyme. Additionally or alternatively, the engineered host cell may be modified to synthetically regulate the expression of the NPI gene in the engineered host cell. In some examples, the engineered host cell may be modified to incorporate a copy, copies, or additional copies, of the NPI gene. Additionally or alternatively, the engineered host cell may be modified to incorporate the introduction of a strong promoter element for the overexpression of the NPI gene within the engineered host cell. In some cases, the NPI gene may be codon optimized for expression in Saccharomyces cerevisiae. The NPI gene may be derived from Papaver somniferum, Papaver bracteatum, Papaver orientale, Papaver spp., or another species.

[COR] In some examples, the engineered host cell may modify the expression of the enzyme codeinone reductase. Codeinone reductase is encoded by the COR gene. In some examples, codeinone reductase catalyzes the reaction of codeinone to codeine, as referenced in FIG. 4. In some cases, codeinone reductase can catalyze the reaction of neopinone to neopine. In other examples, COR can catalyze the reduction of other morphinans including hydrocodone→dihydrocodeine, 14-hydroxycodeinone→14-hydroxycodeine, and hydromorphone →dihydromorphine. The engineered host cell may be modified to include constitutive expression of the COR gene in the engineered host cell. Additionally or alternatively, the engineered host cell may be modified to synthetically regulate the expression of the COR gene in the engineered host cell. In some examples, the engineered host cell may be modified to incorporate a copy, copies, or additional copies, of the COR gene. Additionally or alternatively, the engineered host cell may be modified to incorporate the introduction of a strong promoter element for the overexpression of the COR gene within the engineered host cell. In some cases, the COR gene may be codon optimized for expression in Saccharomyces cerevisiae. Additionally or alternatively, the COR gene may be modified with the addition of targeting sequences for mitochondria, vacuole, endoplasmic reticulum, or a combination thereof. The COR gene may be derived from Papaver somniferum, or another species.

[CODM] In some examples, the engineered host cell may modify the expression of the enzyme codeine O-demethylase. Codeine O-demethylase is encoded by the CODM gene. In some examples, codeine O-demethylase catalyzes the reaction of codeine to morphine, as referenced in FIG. 4. Codeine O-demethylase can also catalyze the reaction of neopine to neomorphine. Codeine O-demethylase can also catalyze the reaction of thebaine to oripavine. In other examples, CODM may catalyze the O-demethylation of BIAs within the 1-benzylisoquinoline, aporphine, and protoberberine classes such as reticuline, isocorydine, and scoulerine, respectively. In other examples, the CODM enzyme may catalyze an O,O-demethylenation reaction to cleave the methylenedioxy bridge structures in protopines. The engineered host cell may be modified to include constitutive expression of the CODM gene in the engineered host cell. Additionally or alternatively, the engineered host cell may be modified to synthetically regulate the expression of the CODM gene in the engineered host cell. In some examples, the engineered host cell may be modified to incorporate a copy, copies, or additional copies, of the CODM gene. Additionally or alternatively, the engineered host cell may be modified to incorporate the introduction of a strong promoter element for the overexpression of the CODM gene within the engineered host cell. In some cases, the CODM gene may be codon optimized for expression in Saccharomyces cerevisiae. Additionally or alternatively, the CODM gene may be modified with the addition of targeting sequences for mitochondria. The CODM gene may be derived from Papaver somniferum, Papaver spp., or another species.

[BBE] In some examples, the engineered host cell may modify the expression of the enzyme berberine bridge enzyme. The berberine bridge enzyme is encoded by the BBE gene. In some examples, berberine bridge enzyme catalyzes the reaction of (S)-reticuline →(S)-scoulerine, as referenced in FIG. 8. FIG. 8 illustrates a biosynthetic scheme for conversion of L-tyrosine to protoberberine alkaloids, in accordance with some embodiments of the disclosure. In particular, FIG. 8 provides the use of the enzymes BBE, berberine bridge enzyme; S9OMT, scoulerine 9-O-methyltransferase; CAS, canadine synthase; CPR, cytochrome P450 reductase; and STOX, tetrahydroprotoberberine oxidase. The engineered host cell may be modified to include constitutive expression of the BBE gene in the engineered host cell. Additionally or alternatively, the engineered host cell may be modified to synthetically regulate the expression of the BBE gene in the engineered host cell. In some examples, the engineered host cell may be modified to incorporate a copy, copies, or additional copies, of the BBE gene. Additionally or alternatively, the engineered host cell may be modified to incorporate the introduction of a strong promoter element for the overexpression of the BBE gene within the engineered host cell. The BBE gene may be derived from Papaver somniferum, Argemone mexicana, Eschscholzia californica, Berberis stolonifera, Thalictrum flavum subsp. glaucum, Coptis japonica, Papaver spp., or another species.

[CYP2D6] In some examples, the engineered host cell may modify the expression of cytochrome P450, family 2, subfamily D, polypeptide 6. This particular cytochrome P450 is encoded by the CYP2D6 gene. This particular cytochrome P450 enzyme may be characterized as a promiscuous oxidase. In some examples, this particular cytochrome P450 enzyme may catalyze the reaction of (R)-reticuline+NADPH+H⁺+O₂→salutaridine+NADP⁺2 H₂O, among other reactions.

[S9OMT] In some examples, the engineered host cell may modify the expression of the enzyme S-adenosyl-L-methionine:(S)-scoulerine 9-O-methyltransferase. S-adenosyl-L-methionine:(S)-scoulerine 9-O-methyltransferase is encoded by the S9OMT gene. In some examples, S-adenosyl-L-methionine:(S)-scoulerine 9-O-methyltransferase catalyzes the reaction of S-adenosyl-L-methionine+(S)-scoulerine →S-adenosyl-L-homocysteine+(S)-tetrahydrocolumbamine, as referenced in FIG. 8. The engineered host cell may be modified to include constitutive expression of the S9OMT gene in the engineered host cell. Additionally or alternatively, the engineered host cell may be modified to synthetically regulate the expression of the S9OMT gene in the engineered host cell. In some examples, the engineered host cell may be modified to incorporate a copy, copies, or additional copies, of the S9OMT gene. Additionally or alternatively, the engineered host cell may be modified to incorporate the introduction of a strong promoter element for the overexpression of the S9OMT gene within the engineered host cell. In some cases, the S9OMT gene may be codon optimized for expression in Saccharomyces cerevisiae. The S9OMT gene may be derived from Thalictrum flavum subsp. glaucum, Coptis japonica, Coptis chinensis, Papaver somniferum, Thalictrum spp., Coptis spp., Papaver spp., or another species. In some examples, the S9OMT gene may be 100% similar to the naturally occurring gene.

[CAS] In some examples, the engineered host cell may modify the expression of the enzyme (S)-canadine synthase. (S)-canadine synthase is encoded by the CAS gene. In some examples, (S)-canadine synthase catalyzes the reaction of (S)-tetrahydrocolumbamine 4 (S)-canadine, as referenced in FIG. 8. The engineered host cell may be modified to express the CAS gene in the engineered host cell. The engineered host cell may be modified to include constitutive expression of the CAS gene in the engineered host cell. Additionally or alternatively, the engineered host cell may be modified to synthetically regulate the expression of the CAS gene in the engineered host cell. In some examples, the engineered host cell may be modified to incorporate a copy, copies, or additional copies, of the CAS gene. Additionally or alternatively, the engineered host cell may be modified to incorporate the introduction of a strong promoter element for the overexpression of the CAS gene within the engineered host cell. The CAS gene may be derived from Thalictrum flavum subsp. glaucum, Coptis japonica, Thalictrum spp., Coptis spp., or another species.

[STOX] In some examples, the engineered host cell may modify the expression of the enzyme (S)-tetrahydroprotoberberine oxidase. (S)-tetrahydroprotoberberine oxidase is encoded by the STOX gene. In some examples, (S)-tetrahydroprotoberberine oxidase catalyzes the reaction of (S)-tetrahydroberberine+2 O₂→berberine+2 H₂O₂, as referenced in FIG. 8. The engineered host cell may be modified to include constitutive expression of the STOX gene in the engineered host cell. Additionally or alternatively, the engineered host cell may be modified to synthetically regulate the expression of the STOX gene in the engineered host cell. In some examples, the engineered host cell may be modified to incorporate a copy, copies, or additional copies, of the STOX gene. Additionally or alternatively, the engineered host cell may be modified to incorporate the introduction of a strong promoter element for the overexpression of the STOX gene within the engineered host cell. In some examples the STOX may be modified at the N-terminus. In some cases, the STOX gene may be codon optimized for expression in Saccharomyces cerevisiae. The STOX gene may be derived from Berberis wilsonae, Coptis japonica, Berberis spp., Coptis spp., or another species.

[morA] In some examples, the engineered host cell may modify the expression of the enzyme morphine dehydrogenase. Morphine dehydrogenase is encoded by the morA gene. In some examples, morphine dehydrogenase catalyzes the reaction of morphine →morphinone, as referenced in FIG. 4. In other examples, morphine dehydrogenase catalyzes the reaction of codeinone →codeine, also as referenced in FIG. 4. FIG. 4 illustrates a biosynthetic scheme for production of semi-synthetic opiods, in accordance with some embodiments of the disclosure. In particular, FIG. 4 illustrates extended transformations of thebaine in yeast by incorporating morA, morphine dehydrogenase; and morB, morphine reductase.

The engineered host cell may be modified to include constitutive expression of the morA gene in the engineered host cell. Additionally or alternatively, the engineered host cell may be modified to synthetically regulate the expression of the morA gene in the engineered host cell. In some examples, the engineered host cell may be modified to incorporate a copy, copies, or additional copies, of the morA gene. Additionally or alternatively, the engineered host cell may be modified to incorporate the introduction of a strong promoter element for the overexpression of the morA gene within the engineered host cell. In some cases, the morA gene may be codon optimized for expression in Saccharomyces cerevisiae. The morA gene may be derived from Pseudomonas putida or another species.

[morB] In some examples, the engineered host cell may modify the expression of the enzyme morphinone reductase. Morphinone reductase is encoded by the morB gene. In some examples, morphinone reductase catalyzes the reaction of codeinone→hydrocodone, as referenced in FIG. 4. In other examples, morphinone reductase catalyzes the reaction of morphinone →hydromorphone, also as referenced in FIG. 4. In other examples, morphinone reductase catalyzes the reaction 14-hydroxycodeinone→oxycodone. The engineered host cell may be modified to include constitutive expression of the morB gene in the engineered host cell. Additionally or alternatively, the engineered host cell may be modified to synthetically regulate the expression of the morB gene in the engineered host cell. In some examples, the engineered host cell may be modified to incorporate a copy, copies, or additional copies, of the morB gene. Additionally or alternatively, the engineered host cell may be modified to incorporate the introduction of a strong promoter element for the overexpression of the morB gene within the engineered host cell. In some cases, the morB gene may be codon optimized for expression in Saccharomyces cerevisiae. The morB gene may be derived from Pseudomonas putida or another species.

[3ODM] In some examples, the engineered host cell may modify the expression of the enzyme 3-O-demethylase. 3-O-demethylase is encoded by the 3ODM gene. In some examples, 3-O-demethylase may catalyze reactions such as oxycodone→oxymorphone; hydrocodone→hydromorphone; dihydrocodeine→dihydromorphine; 14-hydroxycodeine→14-hydroxymorphine; codeinone→morphinone; and 14-hydroxycodeinone→14-hydroxymorphinone, among other reactions.

[BM3] In some examples, the engineered host cell may express the enzyme BM3. BM3 is a Bacillus megaterium cytochrome P450 involved in fatty acid monooxygenation in its native host. In some cases BM3 N-demethylates an opioid to produce a nor-opioid. In some cases the host cell is modified to express BM3 in addition to other heterologous enzymes for the production of a nal-opioid or nor-opioid. The engineered host cell may be modified to include constitutive expression of the BM3 gene in the engineered host cell. Additionally or alternatively, the engineered host cell may be modified to synthetically regulate the expression of the BM3 gene in the engineered host cell. In some examples, the engineered host cell may be modified to incorporate a copy, copies, or additional copies, of the BM3 gene. Additionally or alternatively, the engineered host cell may be modified to incorporate the introduction of a strong promoter element for the overexpression of the BM3 gene within the engineered host cell. BM3 has several advantages as a biosynthetic enzyme including that it is soluble, comes with a fused reductase partner protein, and can readily be engineered to accept new substrates. Additionally, Table 9 illustrates variants of BM3 N-demethylases.

[SFA1] In some examples, the engineered host cell may express the enzyme SFA1. SFA1 may function as a formaldehyde dehydrogenase enzyme that can be used for formaldehyde detoxification in yeast (Achkor, H., Diaz, M., Fernindez, M. R., Biosca, J. A., Pares, X., & Martinez, M. C. (2003). Enhanced formaldehyde detoxification by overexpression of glutathione-dependent formaldehyde dehydrogenase from Arabidopsis. Plant Physiology, 132(4), 2248-2255). This pathway is depicted in FIG. 37. In this pathway, formaldehyde spontaneously conjugates to glutathione, a common yeast metabolite, to form S-hydroxymethyglutathione. SFA1 then oxidizes S-hydroxymethyglutathione to S-formylglutathione. In certain embodiments, the engineered host cell may be modified to include constitutive expression of SFA1 in the engineered host cell. Additionally or alternatively, the engineered host cell may be modified to synthetically regulate the expression of SFA1 in the engineered host cell. In some examples, the engineered host cell may be modified to incorporate a copy, copies, or additional copies, of the SFA1 gene. Additionally or alternatively, the engineered host cell may be modified to incorporate the introduction of a strong promoter element for the overexpression of SFA 1 within the engineered host cell.

[DUG2/DUG3] In some examples, the engineered host cell may be engineered to reduce or eliminate the expression of DUG2 or DUG3. DUG2 and DUG3 are proteins that together form a peptidase complex that cleaves the link between glutamate and cysteine (Baudouin-Cornu, P., Lagniel, G., Kumar, C., Huang, M. E., & Labarre, J. (2012). Glutathione degradation is a key determinant of glutathione homeostasis. Journal of Biological Chemistry, 287(7), 4552-4561.). Both DUG2 and DUG3 are necessary for formation of the active peptidase in yeast. In certain embodiments, the engineered host cell may be modified to delete or reduce the expression of DUG2 and/or DUG3 in the engineered host cell using CRISPR/Cas9 or other gene editing technology. Additionally or alternatively, in some embodiments the engineered host cell may be modified to synthetically regulate the expression of the DUG2 or DUG3 gene in the engineered host cell using synthetic transcription factors and promoters. Additionally or alternatively, the engineered host cell may be modified to incorporate the introduction of a different gene to replace the expression of the DUG2 or DUG3 gene within the engineered host cell using genome editing tools.

[P450] P450s are a superfamily of monooxygenases, which show broad diversity in their reaction chemistry. Certain P450s provide 14-hydroxylase activity. In some embodiments, the engineered host cell may be engineered to express a cytochrome P450 (P450) enzyme conferring C-14-hydroxylase activity. In certain embodiments, the engineered host cell may be modified to include constitutive expression of a P450 in the engineered host cell. Additionally or alternatively, the engineered host cell may be modified to synthetically regulate the expression of a P450 in the engineered host cell. In some examples, the engineered host cell may be modified to incorporate a copy, copies, or additional copies, of a P450 gene. Additionally or alternatively, the engineered host cell may be modified to incorporate the introduction of a strong promoter element for the overexpression of a P450 within the engineered host cell.

[CPR] P450s providing 14-hydroxylase activity commonly require a partner enzyme, a cytochrome P450 reductase (CPR), to provide the necessary reducing power so a P450 can reduce oxygen to produce an active intermediate for hydroxylation. In some embodiments, the engineered host cell may be engineered to express a CPR enzyme. In certain embodiments, the engineered host cell may be modified to include constitutive expression of a CPR in the engineered host cell. Additionally or alternatively, the engineered host cell may be modified to synthetically regulate the expression of a CPR in the engineered host cell. In some examples, the engineered host cell may be modified to incorporate a copy, copies, or additional copies, of a CPR gene. Additionally or alternatively, the engineered host cell may be modified to incorporate the introduction of a strong promoter element for the overexpression of a CPR within the engineered host cell.

Examples of the aforementioned genes can be expressed from a number of different platforms in the host cell, including plasmid (2p, ARS/CEN), YAC, or genome. In addition, examples of the aforementioned gene sequences can either be native or codon optimized for expression in the desired heterologous host (e.g., Saccharomyces cerevisiae).

### EXAMPLES

The following examples are given for the purpose of illustrating various embodiments of the disclosure and are not meant to limit the disclosure in any fashion. Where indicated, expression constructs are understood to incorporate a suitable promoter, gene, and terminator, even if the exact terminator sequence used is not specified. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Bioinformatic Identification of Enzymes for Morphinan Alkaloid Production

The OneKP (Matasci N et al. 2014. Data access for the 1,000 Plants (1KP) project. Gigascience 3:17) and plant transcriptome database was queried with amino acid sequences of representative variants from each of the hypothesized classes of enzymes. In particular, the Papaver genus, which includes many plant species that produce benzylisoquinoline alkaloids of interest, were searched. The list of candidate sequences from these plants were narrowed down using an e-value cutoff of 10⁻⁵⁰ to the representative sequence. For some candidates, the complete sequence was not present in the assembled transcriptome. In these cases, the sequence was completed using raw sequencing reads.

### Example 2: Platform Yeast Strains Engineered to Produce (S)-Reticuline from Glucose and Simple Nitrogen Sources

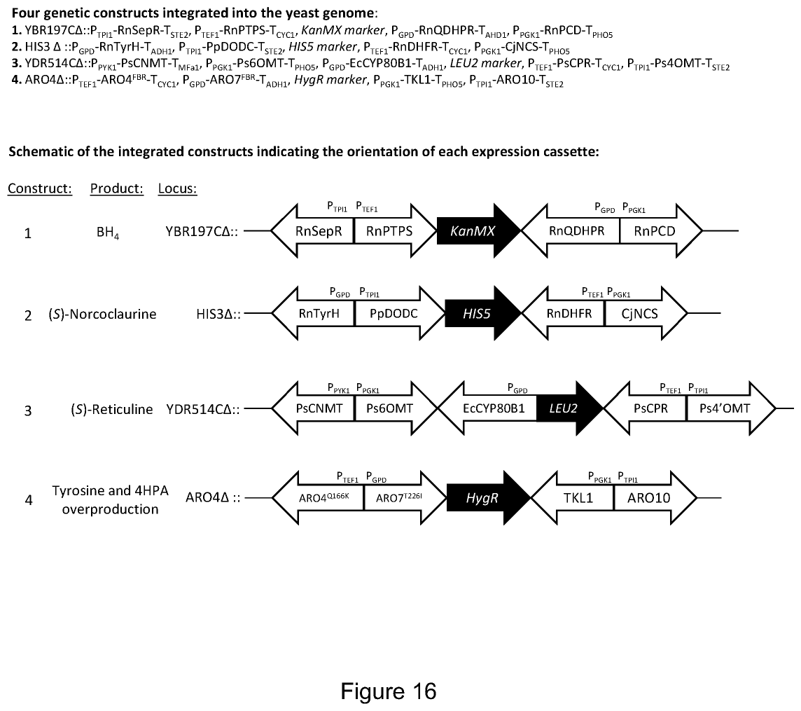

A platform yeast strain that produces the significant branch point BIA intermediate (S)-reticuline from L-tyrosine was constructed (FIG. 16). Specifically, four multi-gene expression constructs were integrated into the genome of a yeast strain. The composition of the four constructs is indicated in FIG. 16. Each construct is comprised of 4 or 5 genes expressed from yeast promoters. Genes are positioned at each locus as complete expression cassettes comprising a promoter, gene open reading frame, and terminator as specified in the annotations above the schematic. The schematic shows the orientation of each expression cassette by the direction of the arrow representing a given gene. Selectable markers are italicized in the annotation and represented by grey arrows in the schematic. Each selection marker is flanked by loxP sites to allow removal of the marker from the locus. Additionally, each construct has a selectable marker flanked by loxP sites so that it can be removed by Cre recombinase.

In the first integration construct, four heterologous genes from Rattus norvegicus are integrated into the YBR197C locus together with a G418 selection marker (KanMX). RnPTPS, RnSepR, RnPCD, and RnQDHPR are required to synthesize and regenerate tetrahydrobiopterin (BH₄) from the yeast endogenous folate synthesis pathway as indicated in FIG. 2. Each gene is codon optimized for expression in yeast.

In the second integration construct, four heterologous genes are integrated into the HIS3 locus together with the HIS5 selection marker. Rattus norvegicus tyrosine hydroxylase (RnTyrH) converts tyrosine to L-DOPA using the cosubstrate BH₄ generated by the preceding integration construct. The RnTyrH gene can be any of the wild-type or improved mutants which confer enhanced activity (e.g., W166Y, R37E, and R38E). A second Rattus norvegicus gene, RnDHFR, encodes an enzyme that reduces dihydrobiopterin (an oxidation product of BH₄) to BH₄, in this way increasing the availability of this cosubstrate. Also included in the third construct is PpDODC from Pseudomonas putida, an enzyme that converts L-DOPA to dopamine. The fourth enzyme is CjNCS from Coptis japonica, which condenses 4-HPAA and dopamine to make norcoclaurine. Each gene is codon optimized for expression in yeast.

In the third integration construct, five heterologous genes from plants and the LEU2 selection marker are integrated into the locus YDR514C. Ps60MT, Ps4′OMT, and PsCNMT are methyltransferases from Papaver somniferum and are expressed as native plant nucleotide sequences. A fourth P. somniferum gene, yPsCPRv2, is codon optimized for yeast and encodes a reductase that supports the activity of a cytochrome P450 from Eschscholzia californica, EcCYP80A1. The enzymes encoded in this construct perform two O-methylations, an N-methylation, and a hydroxylation to produce reticuline from the norcoclaurine produced by the preceding integration construct. Each gene is codon optimized for expression in yeast.

In the final integration construct, additional copies of Saccharomyces cerevisiae endogenous genes ARO4^{Q166K}, ARO7^{T226}, TYR1, and ARO10 are integrated into the ARO4 locus together with a hygromycin resistance selection marker. ARO4^{Q166K} and ARO7^{T2261} are feedback-resistant mutants of ARO4 and ARO7 which each encode a single base pair substitution relative to the wild-type sequence. TYR1 and ARO10 are identical to the native yeast genes, but are expressed behind strong promoters. Aro4p and Aro7p are enzymes in the biosynthesis of aromatic amino acids including tyrosine. Removing feedback inhibition from these enzymes results in upregulation of endogenous tyrosine biosynthesis. Overexpression of Tyr1p upregulates tyrosine biosynthesis and thus production of tyrosine. Overexpression of Aro10p increases the production of 4-HPA.

Platform yeast strains can be constructed with any number of the four expression cassettes. Specifically, platform yeast strains were constructed with integration constructs 1-4 and integration constructs 1-3. In the latter strain in which the tyrosine over-production construct (construct 4) is excluded, additional tyrosine may be supplied in the culture medium to support the biosynthesis of reticuline. Additional genetic modifications may be incorporated into the platform strains to support production of downstream BIAs and increased flux to BIA biosynthesis.

The yeast strains were grown in synthetic complete media with the appropriated amino acid drop out solution at 28° C. BIA metabolites in the media supernatant were analyzed after 48 and 96 hours of growth by LC-MS/MS analysis.

### Example 3: Platform Yeast Strains Engineered to Produce Thebaine from Glucose and Simple Nitrogen Sources

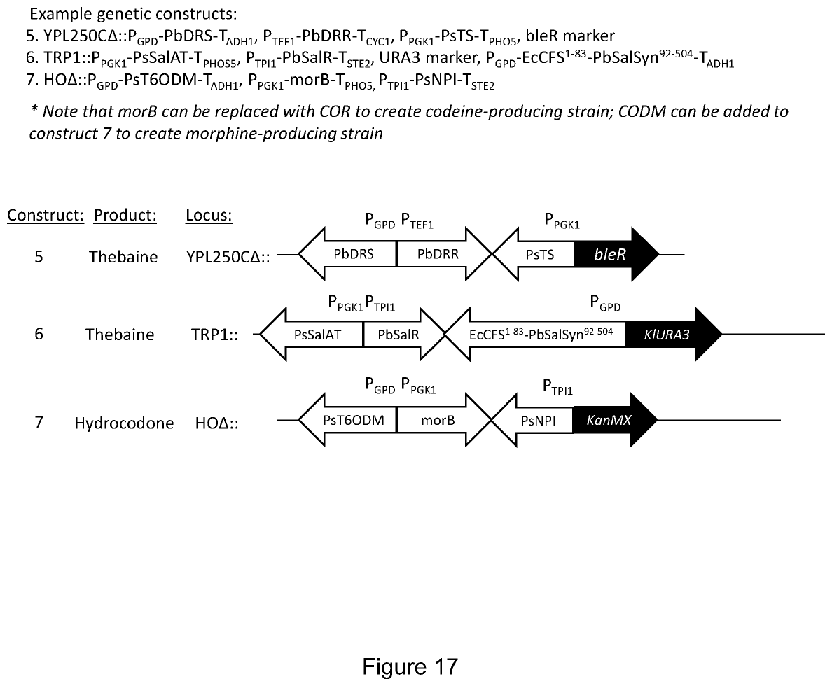

Yeast strains can be engineered for the production of the morphinan alkaloid thebaine from early precursors such as tyrosine. As an example, the platform yeast strains described in Example 2 can be further engineered to produce the morphinan alkaloid products from L-tyrosine (FIG. 17).

The platform yeast strain producing (S)-reticuline from L-tyrosine (see description in Example 2) was further engineered to incorporate an engineered split epimerase DRS-DRR, an engineered salutaridine synthase, salutaridine reductase, salutaridinol acetyltransferase, and thebaine synthase to convert the biosynthesized (S)-reticuline to the first morphinan alkaloid thebaine (FIG. 4). Three expression cassettes (P_{TDH3}-yEcCFS¹⁻²⁶-yPbSS³³⁻⁵⁰⁴, P_{TP11}-yPbSalR, P_{TEF1}-yPsSalAT) were assembled into an integration construct with a URA3 selective marker and integrated into the locus TRP1 in the platform yeast strain. An additional three expression cassettes (P_{TDH3}-yPbDRS, P_{TEF1}-yPbDRR, P_{PGK1}-yPsTS) were assembled into an integration construct with a bleR selective marker and integrated into the locus YPL250CA in the platform yeast strain. The composition of the two constructs is indicated in FIG. 17.

The yeast strains harboring the integrated cassettes were grown in synthetic complete media with the appropriated drop out solution at 28° C. After 96 hours of growth, the media was analyzed for BIA metabolites by LC-MS/MS analysis.

### Example 4: Yeast Strains Engineered to Produce Downstream Morphinan Alkaloids from Glucose and Simple Nitrogen Sources

Yeast strains can be engineered for the production of the downstream morphinan alkaloids from early precursors such as tyrosine. As an example, the platform yeast strains described in Example 3 can be further engineered to produce the downstream morphinan alkaloid products from L-tyrosine (FIG. 4).

The platform yeast strain producing thebaine from L-tyrosine (see description in Example 3) was further engineered to incorporate thebaine 6-O-demethylase, neopinone isomerase, codeinone reductase, and codeinone-O-demethylase to convert the biosynthesized thebaine to the downstream morphinan alkaloids including morphine (FIG. 17). Four expression cassettes (P_{GPD}-T6ODM, P_{PGK1}—COR, P_{ADH1}-CODM, P_{TP11}-yPsNPI) were directly assembled with a KanMX selective marker and integrated into the HOA locus in the thebaine platform yeast strain to create a morphine-producing yeast strain (Thodey et al., 2014). Three expression cassettes (P_{GPD}-T6ODM, P_{PGK1}—COR, P_{TP11}-yPsNPI) were directly assembled with a KanMX selective marker and integrated into the HOA locus in the thebaine platform yeast strain to create a codeine-producing yeast strain.

The yeast strains harboring the integrated cassettes were grown in synthetic complete media with the appropriated drop out solution at 28° C. After 96 hours of growth, the media was analyzed for BIA metabolites by LC-MS/MS analysis.

### Example 5: Yeast Strains Engineered to Produce Semi-Synthetic Opioids from Glucose and Simple Nitrogen Sources

Yeast strains can be engineered for the production of the downstream semi-synthetic morphinan alkaloids from early precursors such as tyrosine. As an example, the yeast strains described in Examples 3 and 4 can be further engineered to produce the semi-synthetic opioid products from L-tyrosine (FIG. 4).

The yeast strains producing thebaine from L-tyrosine (see description in Examples 3 and 4) were further engineered to incorporate thebaine 6-O-demethylase, neopinone isomerase, and morphinone reductase to convert the biosynthesized thebaine to the semi-synthetic morphinan alkaloid hydrocodone (FIG. 17). Three expression cassettes (P_{GPD}-T6ODM, P_{PGK1}-morB, P_{TP11}-yPsNPJ) were directly assembled with a KanMX selective marker and integrated into the HOA locus in the thebaine platform yeast strain to create a hydrocodone-producing yeast strain (Thodey et al., 2014).

The yeast strains harboring the integrated cassettes were grown in synthetic complete media with the appropriated drop out solution at 28° C. After 96 hours of growth, the media was analyzed for BIA metabolites by LC-MS/MS analysis.

### Example 6: Production of Downstream Morphinan Alkaloids from Glucose and Simple Nitrogen Sources Via Engineered Yeast Strains

Yeast strains were engineered as described in Examples 2, 3, and 4 to produce the downstream morphinan alkaloids codeine and morphine directly from simple sugars (e.g., glucose) and nitrogen sources present in standard growth media. Specifically, a CEN.PK strain of Saccharomyces cerevisiae was engineered to express the following heterologous enzymes via integration into the yeast chromosome: TyrH, DODC, PTPS, SepR, PCD, QDHPR, NCS, 60MT, CNMT, CYP80B1, CPR, 40MT, DRS, DRR, SalSyn, SalR, SalAT, TS, T6ODM, COR (variant 1.3, SEQ ID NO. 87). A version of this yeast strain was also engineered to express CODM via integration into the yeast chromosome. In this example, the SalSyn enzyme is engineered to have its leader sequence replaced with 83 amino acids from the N-terminus of Eschscholzia californica chelanthifoline synthase (EcCFS). Additional modifications were made to the strain to increase BIA precursor accumulation, including: overexpression of ARO10, overexpression of TYR1, expression of a feedback resistant ARO4 (ARO4^{Q166K}), and expression of a feedback resistant ARO7 (ARO7^{T2261}). Separate engineered yeast strains were made as described, harboring different variants of enzymes encoding neopinone isomerase activity (NPI), including SEQ ID NO. 83, which is a variant of SEQ ID NO. 82 with a N-terminal truncation of the first 18 amino acids (i.e., NPI (truncated)), and no neopinone isomerase enzyme (codeine-producing strain: YA1033; morphine-producing strain: YA1022). The sequences of the enzyme variants are provided in Table 3.

The described yeast strains were inoculated into 2 ml of synthetic complete media (yeast nitrogen base and amino acids) with 2% glucose and grown for approximately 4 hours at 28° C. Then, 10 μL of each culture was transferred to 400 μL of fresh media in a 96-well plate in replicates of 4 and grown for an additional 48 hours at 28° C. The production media contains 1× yeast nitrogen broth and amino acids, 20 mM ascorbic acid, 300 mg/L tyrosine, 40 g/L maltodextrin, and 2 units/L amylase. The amylase is used to mimic a fed-batch process and gradually releases glucose from maltodextrin polymer so that the yeast can use it as a carbon source. The cells were separated from the media by centrifugation, and thebaine concentration was measured directly in the supernatant by LC-MS/MS analysis.

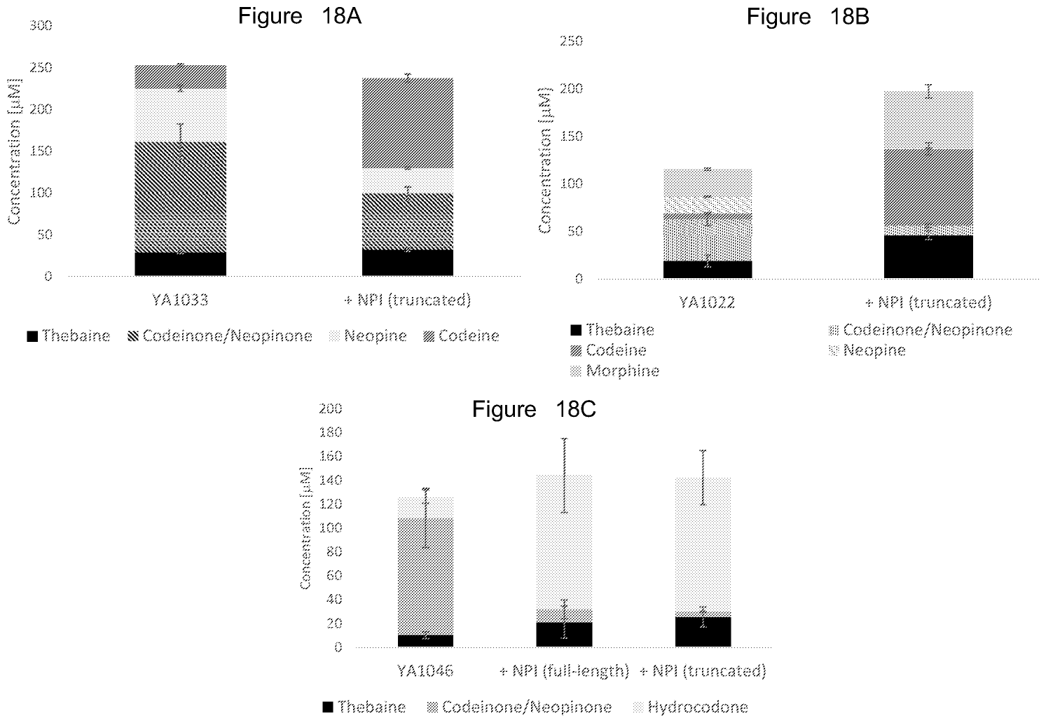

Engineered codeine-producing yeast strains produced thebaine, codeine, and other benzylisoquinoline alkaloids from glucose and simple nitrogen sources present in the growth media (FIG. 18A). Engineered morphine-producing yeast strains produced thebaine, codeine, morphine, and other benzylisoquinoline alkaloids from glucose and simple nitrogen sources present in the growth media (FIG. 18B).

### Example 7: Production of Downstream Semi-Synthetic Opioids from Glucose and Simple Nitrogen Sources Via Engineered Yeast Strains

Yeast strains were engineered as described in Examples 2, 3, 4, and 5 to produce the downstream semi-synthetic opioid hydrocodone directly from simple sugars (e.g., glucose) and nitrogen sources present in standard growth media. Specifically, a CEN.PK strain of Saccharomyces cerevisiae was engineered to express the following heterologous enzymes via integration into the yeast chromosome: TyrH, DODC, PTPS, SepR, PCD, QDHPR, NCS, 60MT, CNMT, CYP80B1, CPR, 40MT, DRS, DRR, SalSyn, SalR, SalAT, TS, T6ODM, morB. In this example, the SalSyn enzyme is engineered to have its leader sequence replaced with 83 amino acids from the N-terminus of Eschscholzia californica chelanthifoline synthase (EcCFS). Additional modifications were made to the strain to increase BIA precursor accumulation, including: overexpression of ARO10, overexpression of TYR1, expression of a feedback resistant ARO4 (ARO4^{Q166K}), and expression of a feedback resistant ARO7 (ARO7^{T2261}) Separate engineered yeast strains were made as described, harboring different variants of enzymes encoding neopinone isomerase activity (NPI), including SEQ ID NO. 54 (i.e., NPI (full-length)) and SEQ ID NO. 55, which is a variant of SEQ ID NO. 56 with a N-terminal truncation of the first 18 amino acids (i.e., NPI (truncated)), and no neopinone isomerase enzyme (YA1046). The sequences of the enzyme variants are provided in Table 3.

The described yeast strains were inoculated into 2 ml of synthetic complete media (yeast nitrogen base and amino acids) with 2% glucose and grown for approximately 4 hours at 28° C. Then, 10 μL of each culture was transferred to 400 μL of fresh media in a 96-well plate in replicates of 4 and grown for an additional 48 hours at 28° C. The production media contains 1× yeast nitrogen broth and amino acids, 20 mM ascorbic acid, 300 mg/L tyrosine, 40 g/L maltodextrin, and 2 units/L amylase. The amylase is used to mimic a fed-batch process and gradually releases glucose from maltodextrin polymer so that the yeast can use it as a carbon source. The cells were separated from the media by centrifugation, and thebaine concentration was measured directly in the supernatant by LC-MS/MS analysis.

Engineered hydrocodone-producing yeast strains produced thebaine, hydrocodone, and other benzylisoquinoline alkaloids from glucose and simple nitrogen sources present in the growth media (FIG. 18C).

### Example 8: Norcoclaurine Synthase Activity in Microbial Strains

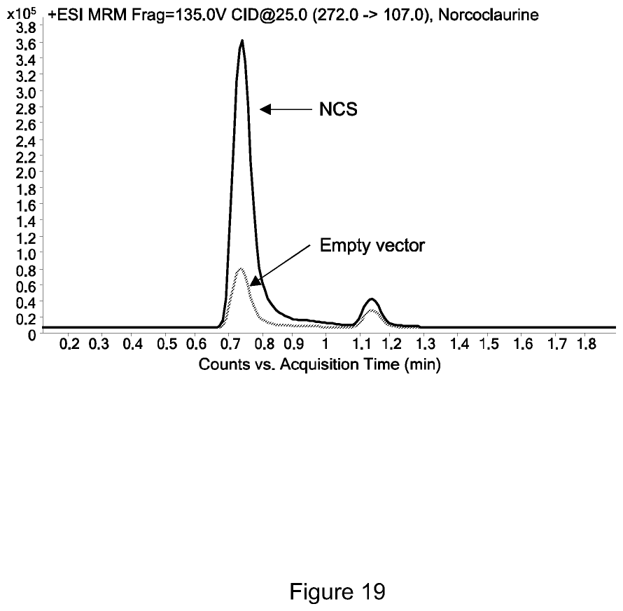

To engineer norcoclaurine production in yeast we: (1) introduced a heterologous NCS enzyme on a plasmid, (2) supplied tyrosine in the culture medium to promote 4-HPAA production by the host cell's native tyrosine catabolic pathway, and (3) supplied dopamine directly in the culture medium to provide the second substrate for NCS activity. CEN.PK2 was transformed with either a yeast shuttle plasmid containing NCS (SEQ ID NO: 70) cloned in an expression cassette with a P_{TDH3} promoter and T_{CYC1} terminator, or with an empty vector in which there was no gene inserted between the promoter and terminator. Cells were transformed using the standard lithium acetate technique, plated on selective solid medium, and cultured 2 days at 30° C. Colonies were picked at random and cultured first in standard synthetic complete (SC) dropout liquid medium for 48 hours at 30° C. with 300 rpm agitation. The stationary phase cultures were then backdiluted 100× into SC media supplemented with 0 or 100 mM dopamine and 300 mg/L tyrosine and cultured a further 48 hours at 30° C., 300 rpm. When the cells were pelleted and the culture medium analyzed by LCMS we observed that the empty vector control strain (no-enzyme control) biosynthesized and exported 21.9 μM norcoclaurine into the culture medium when supplied with 100 mM dopamine (FIG. 19). This demonstrated that wild type yeast can take up dopamine from the culture medium to support a low-level of spontaneous norcoclaurine production by the condensation of endogenous 4-HPAA and exogenous dopamine. However, the engineered strain expressing an active NCS enzyme and supplied with 100 mM dopamine in the culture medium produced 72.1 μM norcoclaurine, more than 3-fold greater than observed in the no-enzyme control, demonstrating that NCS is active in yeast and catalyzes the formation of norcoclaurine (FIG. 19).

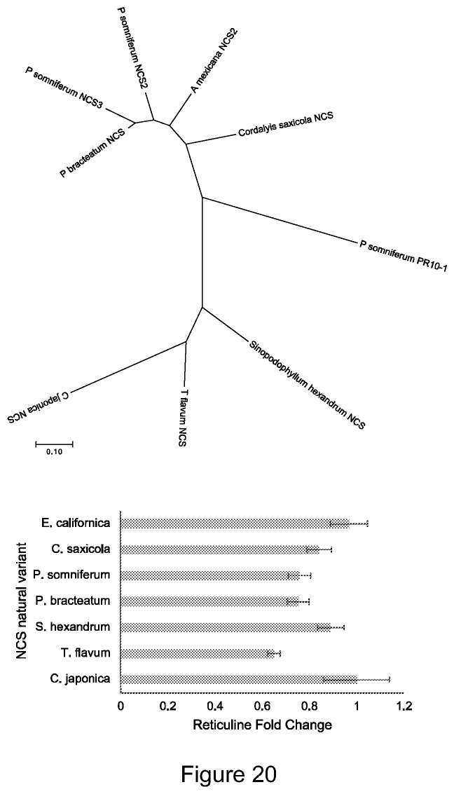

To investigate NSC natural diversity, plant Bet v I proteins from Coptis japonica, Thalictrum flavum, Argemone mexicana, Sinopodophyllum hexandrum, Papaver bracteatum, Papaver somniferum, and. Cordalyis saxicola (Table 5) were identified from the public transcriptomics databases and expressed in yeast. The open ready frames of each gene were first codon optimized for Saccharomyces cerevisiae and then synthesized with 30 base pair overlaps to the P_{TDH3} promoter and T_{CYC1} terminator of the yeast shuttle vector. The genes were then individually transformed into yeast strain YA139 (harboring a complete biosynthetic pathway to reticuline, but lacking NCS activity) together with linearized vector. On transformation, each NCS was incorporated into the plasmid by gap repair creating a construct in which NCS expression was driven by the P_{TDH3} promoter. After two days incubation at 30° C. on solid selective medium individual colonies were picked and assayed for NCS activity. Strains were cultured first in standard SC medium for 48 hours at 30° C. and then backdiluted 100× into SC media supplemented with 200 mg/L tyrosine and cultured a further 48 hours at 30° C. Because strain YA 139 encodes a complete heterologous pathway to reticuline (with the exception of a functional NCS enzyme) the spent culture medium was analyzed by LCMS for the production of the final end product, reticuline. All tested NCS proteins were observed to be active and catalyzed the formation of norcoclaurine which was incorporated into reticuline and exported into the culture medium (FIG. 20).

TABLE 5
Norcoclaurine synthase sequences.

| SEQ ID | | | Species |
|---|---|---|---|
| NO | Name | Amino acid sequence | of origin |
| 69 | CjNCS full | MRMEVVLVVFLMFIGTINCERLIFNGRPLLHRVTKEETVMLYH | Coptis |
| | length | ELEVAASADEVWSVEGSPELGLHLPDLLPAGIFAKFEITGDGGE | japonica |
| | | GSILDMTFPPGQFPHHYREKFVFFDHKNRYKLVEQIDGDFFDLG | |
| | | VTYYMDTIRVVATGPDSCVIKSTTEYHVKPEFAKIVKPLIDTVPL | |
| | | AIMSEAIAKVVLENKHKSSE | |
| 70 | NCS parent | MNGRPLLHRVTKEETVMLYHELEVAASADEVWSVEGSPELGL | C. japonica |
| | | HLPDLLPAGIFAKFEITGDGGEGSILDMTFPPGQFPHHYREKFVF | |
| | | FDHKNRYKLVEQIDGDFFDLGVTYYMDTIRVVATGPDSCVIKST | |
| | | TEYHVKPEFAKIVKPLIDTVPLAIMSEAIAKVVLENKHKSSE | |
| 71 | NCS1 | MNGRPLLHRVTKEETVMLYHELEVAASADEVWSVEGSPELGL | C. japonica |
| | | HLPDLLPAGIFAKFEITGDGGEGSILDMTFPPGQFPHHYREKFVF | |
| | | FDHKNRYKLVEQIDGDFFDLGVTYYMDTIRVVATGPDSCVIKST | |
| | | TEYHVKPEFAKIVKPLIDTVPLANMSEAIAKVVLENKHKSSE | |
| 72 | NCS2 | MNGRPLLHRVTKEETVMLYHELEVAASADEVWSVEGSPELGL | C. japonica |
| | | HLPDLLPAGIFAKFEITGDGGEGSILDMTFPPGQFPHHYREKFVF | |
| | | FDHKNRYKLVEQIDGDFFDLGVTYYMDTIRVVATGPDSCVIKST | |
| | | TEYHVKPEFAKIVKPLITTVPLANMSEAIAKVVLENKHKSSE | |
| 73 | NCS3 | MNGRPLLHRVTKEETVMLYHELEVAASADEVWSVEGSPELGL | C. japonica |
| | | HLPDLLPAGIFAKFEITGDGGEGSILDITFPPGQFPHHYREKFVF | |
| | | FDHKNRYKLVEQIDGDFFDLGVTYYMDTIRVVATGPDSCVIKST | |
| | | TEYHVKPEFAKIVKPLITTVPLANMSEAIAKVVLENKHKSSE | |
| 74 | TfNCS | MMKMEVVFVFLMLLGTINCQKLILTGRPFLHHQGIINQVSTVTK | Thalictrum |
| | PDB: | VIHHELEVAASADDIWTVYSWPGLAKHLPDLLPGAFEKLEIIGD | flavum |
| | 5N8Q | GGVGTILDMTFVPGEFPHEYKEKFILVDNEHRLKKVQMIEGGYL | |
| | | DLGVTYYMDTIHVVPTGKDSCVIKSSTEYHVKPEFVKIVEPLITT | |
| | | GPLAAMADAISKLVLEHKSKSNSDEIEAAIITV | |
| 75 | TfNCS | MMKMEVVFVFLMLLGTINCQKLILTGRPFLHHQGIINQVSTVTK | Thalictrum |
| | | VIHHELEVAASADDIWTVYSWPGLAKHLPDLLPGAFEKLEIIGD | flavum |
| | | GGVGTILDMTFVPGEFPHEYKEKFILVDNEHRLKKVQMIEGGYL | |
| | | DLGVTYYMDTIHVVPTGKDSCVIKSSTEYHVKPEFVKIVEPLITT | |
| | | GPLAAMADAISKLVLEHKSKSNSDEIEAAIITV | |
| 76 | AmNCS | MSKLITTAPLKSMSEAIANYVLKKQSVIRKVVTYELEVPASADSI | Argemone |
| | | WAVYSSPNIPTLLRDVLLPGVFEKLDVIEGNGGVGTVLDIVFPPG | mexicana |
| | | AVPRRYKEKFVKINNEKRLKEVIMIEGGYLDMGCTSYMDRIHVL | |
| | | EKTPNSCVIESSIIYEVKQEYADEMSKLITTVPLKSMSEVIANYVL | |
| | | KKQFRVFGYEIKPKLGLSLLLCLIICLVILGGLLIAGVPV | |
| 77 | ShNCS | MRMEIVSLFLFFLACGTSQGRPLLESKLYRKSTIQKVLHHESTVA | Sinopodophyllum |
| | | ASAEEVWQVYSSPELPKHLSELLPGAFEKVEIFGDGGVGTILEM | hexandrum |
| | | VFPPGEIPQSYKEKFVLIDDERRLKKVEMIEGGYLDMGVTFYMD | |
| | | TIHIVATGCNSCIIKSSTEYYVKPEFADKVQPLITTAPLEAMDEAI | |
| | | TKIVLANKNKSLIEI | |
| 78 | PbNCS | MMRKVIKYDMEVATSADSVWAVYSSPDIPRLLRDVLLPGVFEK | Papaver |
| | | LDVIEGNGGVGTVLDIAFPPGAVPRSYKEKFVNIDRVKRLKEVI | bracteatum |
| | | MIEGGYLDMGCTFYLDRIHVVEKTPNSCVIESSIIYEVKEEFADK | |
| | | MAKLITTEPLQSMAEVISGYVLKKRLQVFGFEIKPNLRFNLLLCL | |
| | | IICLVIAGGMLIGRVP | |
| 79 | PsNCS2 | MSKLITTEPLKSMAEVISNYVIQRESFSARNILNKNSLVKKEIRY | Papaver |
| | | DLEVPTSADSIWSVYSCPDIPRLLRDVLLPGVFQKLDVIEGNGGV | somniferum |
| | | GTVLDIVFPPGAVPRSYKEKFVNINHEKRLKEVIMIEGGYLDMG | |
| | | CTFYMDRIHIFEKTPNSCVIESSIIYEVKEEYAGKMAKLITTEPLE | |
| | | SMAEVISGYVLKKRLQVFGFEIKPKLRFNLLLCLIICLVIAGGMFV | |
| | | AGVPL | |
| 80 | PsNCS3 | MRKVIKYDMEVAVSADSVWAVYSSPDIPRLLRDVLLPGVFEKL | P. somniferum |
| | | DVIEGNGGVGTVLDIVFPPGAVPRSYKEKFVNIDREKRLKEVIMI | |
| | | EGGYLDMGCTFYLDRIHVVEKTKSSCVIESSIVYDVKEECADAM | |
| | | SKLITTEPLKSMAEVISNYVIQKELFSARNILSKQSVVKKEIRYDL | |
| | | EVPISVDSIWSVYSCPDIPRLL | |
| 81 | CsNCS | MGKRIQKKEAKKILRKELTHELEVPTSADSIWAVYGSPDIPRLL | Cordalyis |
| | | RDVLLPGVFEKLDIIEGNGGVGTVLDIAFPPGTVPRSYKEKFVKV | saxicola |
| | | DHDKHLKEVVMIEGGYLDLGCTFYMDRIHVLPKGPNSCVIESSL | |
| | | IYEVREELADAVGSLISIEPLASMAEVVSSYVLKQQLRVFGVVVQ | |
| | | PRVGLSLLLCLILCLVILGGLLIGGVSI | |
| 82 | PsPR10-1 | MRYELINEFDVGASADDVWAVYSSPDLPRLIVELLPGVFKKIDV | P. somniferum |
| | | VEGNGGLGTVLHLVYPHGSVPLSYKEKFVTMDNHKRLKEVRQI | |
| | | EGGYLEMGCTFYMDSFQILKKTHDSCTIRSITKYEVSAELAEKVS | |
| | | SHISVESLVDMARAISKYVIQDNEKNKAEDPSTCSEEGHDKKHG | |
| | | HLHRIWISLFGK | |

### Example 9: Microbial Strains with Enhanced Production of 4-HPAA

To support the production of norcoclaurine by yeast fermentation, strains were engineered to enhance the supply of 4-HPAA from the host cell metabolism. Yeast produce 4-HPAA from 4-hydroxyphenylpyruvate (4HPP) when tyrosine is catabolized in the Ehrlich pathway. Therefore, to enhance production of 4-HPAA we: (1) enhanced flux through the shikimate pathway to upregulate production of 4HPP and tyrosine, and (2) overexpressed ARO10, the enzyme responsible for the irreversible conversion of 4HPP to 4-HPAA.

A yeast strain was engineered with modifications to the chromosomal loci encoding shikimate pathway enzymes Aro4p, Aro7p, and Tyr1p, and Ehrlich pathway enzyme Aro10p (FIG. 1). Specifically, using homologous recombination we introduced mutations ARO4^{Q166K} and ARO7^{T2261} to relieve tyrosine feedback inhibition of these enzymes. We further modified the upstream regions of the TYR1 gene and ARO10 gene to replace the native promoters with the P_{TDH3} and P_{GAL7} promoters, respectively. These promoter swaps removed the native regulation and introduced promoters that drive constitutive expression when yeast are cultured in medium with select carbon sources. Additionally, the GAL80 gene was deleted to allow for constitutive expression of P_{GAL7}-ARO10 in the presence of glucose. The 4-HPAA-engineered strain and a control strain were transformed with a plasmid encoding NCS and the resulting colonies were cultured 48 hours in standard SC media. The stationary phase cultures were then backdiluted 100× into SC media supplemented with 0 or 100 mM dopamine and cultured a further 48 hours. When the culture medium was analyzed by LCMS the 4-HPAA-engineered strain was observed to produce 3- to 4-fold more norcoclaurine relative to the control strain.

### Example 10: Microbial Strains that Biosynthesizes Dopamine

To support the production of norcoclaurine by yeast fermentation, strains were engineered to produce its second substrate, dopamine. In mammalian cells dopamine is synthesized from tyrosine, with either 3,4-dihydroxy-L-phenylalanine (L-DOPA) or tyramine as intermediates. To re-construct the mammalian biosynthetic pathway in yeast we first engineered a functional tyrosine hydroxylase (TyrH) system to convert tyrosine to L-DOPA. TyrH requires a cofactor, tetrahydrobiopterin (BH₄), that is not produced naturally in yeast. Therefore, we introduced a four-enzyme system into yeast to: (1) convert dihydroneopterin triphosphate from the native pathway for tetrahydrofolate synthesis into BH₄, and (2) return BH₄ to its reduced state after it is oxidized by TyrH. Rat BH₄ biosynthesis genes 6-pyruvoyl-tetrahydropterin synthase (RnPTPS) and sepiapterin reductase (RnSepR) were integrated into the yeast genome together with rat BH₄ recycling genes pterin-4alpha-carbinolamine dehydratase (RnPCD) and quinonoid dihydropteridine reductase (RnQDHPR). Next we integrated a human TyrH gene encoding mutations HsTyrH^{W166Y,R37E,R38E} to relieve substrate inhibition by tyrosine. Finally, we integrated a bacterial DOPA decarboxylase from Pseudomonas putida, PpDODC, to complete the pathway from tyrosine to dopamine. All heterologous enzymes were codon optimized for expression in S. cerevisiae and expressed from yeast high-expression or constitutive-expression promoters.

The dopamine engineering described above was combined with the 4-HPAA engineering from Example 9 in a strain expressing an NCS variant (FIG. 1). This norcoclaurine total biosynthesis strain and a control strain were cultured 48 hours in standard SC media. The stationary phase cultures were then backdiluted 100× into SC media supplemented with 300 mg/L tyrosine and 20 mM ascorbic acid and cultured a further 48 hours. The cells were pelleted and the culture medium analyzed by LCMS. L-DOPA, dopamine, and norcoclaurine were all observed in the culture medium from the norcoclaurine total biosynthesis strain, but not in the culture medium from the control strain.

### Example 11: Improving NCS Activity by N-Terminal Truncation

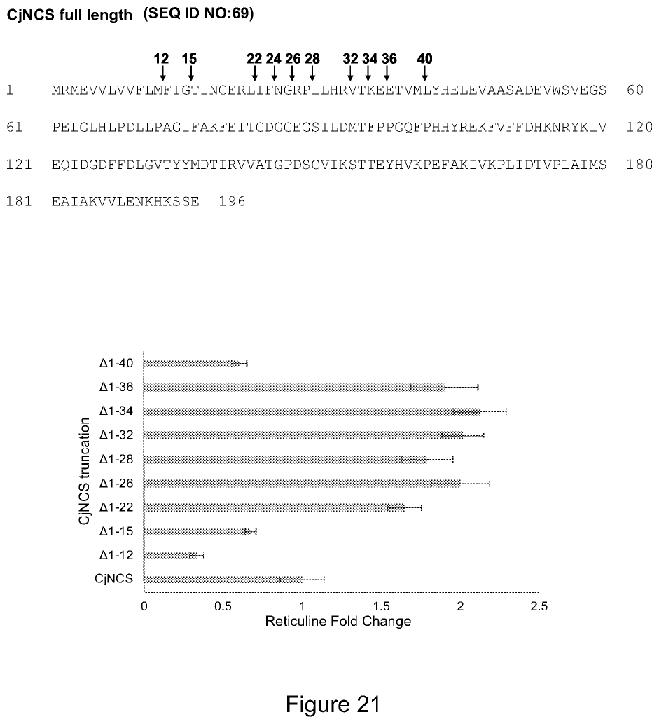

CjNCS (SEQ ID NO: 69) contains a hydrophobic domain in the first 24 amino acids of the N-terminus. This domain could represent a signal peptide, a transmembrane or membrane-interacting domain, or a protein-protein interaction domain. Alternatively, this region could be involved in the regulation or catalytic function of the enzyme. To determine if NCS activity could be enhanced by removal of the N-terminal region, we made deletions of the first 12 to 40 amino acid residues (FIG. 21). The CjNCS template was PCR amplified with oligos to remove the first 12, 15, 22, 26, 28, 32, 34, 36, or 40 amino acids (while replacing the methionine start codon) and introduce 30 base pair overlaps to the P_{TDH3} promoter and T_{CYC1} terminator of a yeast shuttle vector. The PCR products were each transformed together with linearized vector into yeast strains encoding the 4-HPAA and dopamine engineering described in Examples 9 and 10 (FIG. 1). The strains further encoded a heterologous pathway from norcoclaurine to reticuline comprised of P. somniferum norcoclaurine 6-O-methyltransferase (Ps60MT), P. somniferum coclaurine N-methyltransferase (PsCNMT), P. bracteatum N-methylcoclaurine-3′-hydroxylase (PbCYP80B1), P. somniferum cytochrome P450 reductase (PsCPR) and P. somniferum 3′-hydroxy-N-methylcoclaurine 4′-O-methyltransferase (Ps40MT) (FIG. 1).

Strains expressing full-length and N-terminal truncated CjNCS variants were cultured 48 hours in standard SC dropout medium, 30° C., at 300 rpm. The stationary phase cultures were then backdiluted 100× into SC media supplemented with 200 mg/L tyrosine and 10 mM ascorbic acid and cultured a further 48 hours. Cells were pelleted and the culture medium was analyzed by LCMS. Strains expressing the truncated CjNCS variants were observed to produce different titers of reticuline relative to the full-length CjNCS control strain. Truncation of 12 or 15 amino acids from the N-terminus of CjNCS resulted in titers 3-fold and 1.5-fold lower than full-length CjNCS (FIG. 21). In contrast, truncation of 22, 26, 28, 32, 34, or 36 CjNCS N-terminal amino acids increased reticuline titers to 1.6- to 2.1-fold that of the full-length enzyme. Further truncation of 40 amino acid residues caused a drop in reticuline titers.

### Example 12: Improving NCS Activity by Mutagenesis

A directed evolution campaign was carried out using a 24 amino acid N-terminally deleted (Δ1-24) Coptis japonica NCS sequence (SEQ ID NO: 70) as a starting template. The purpose of the screen was to identify residues in any Bet v I fold protein that can be modified to enhance activity. A pool of randomly mutated NCS variants was generated by error-prone PCR to incorporate base pair mutations at a rate of 1-4 bp changes per gene (the NCS open reading frame is 522 bp including the start and stop codons). The oligos were designed to introduce 30 bp overlaps to the P_{TDH3} promoter and T_{CYC1} terminator of a yeast shuttle vector. The mutagenized PCR products were transformed into YA139 together with linearized vector to generate a library of NCS variants by gap repair. A control PCR was also set up with the same NCS template and oligos but using a high-fidelity polymerase for amplification in place of the error-prone polymerase. The non-mutagenized PCR product and linear vector were transformed into YA139 in a second transformation to generate a control strain expressing the parent NCS sequence. Strain YA139 contains the 4-HPAA, dopamine, and reticuline engineering from Examples 2-4 above, but lacks a functional NCS enzyme. Individual colonies were picked and cultured in 96-well microtiter plates in 400 μL standard SC dropout medium at 30° C. with 300 rpm shaking. After 48 hours the wells were backdiluted 100× into 400 μL standard SC dropout medium without supplementation and incubated again at 30° C. with 300 rpm agitation. 48 hours following dilution the plates were centrifuged to pellet the cells and the culture medium in each well analyzed by LCMS to determine the reticuline titer.

Final reticuline titers were compared between strains expressing the NCS mutagenesis library variants and the control strain expressing parent NCS (8 wells of every 96-well plate were reserved for “parent”). For every well determined to have enhanced reticuline titer, 100 μL of yeast cells was removed and used to prepare plasmid. The plasmids were then transferred to Escherichia coli for amplification and sequencing. Many NCS variants contained multiple mutations, some of them silent. To identify which mutations were causal, oligos were ordered to recreate the individual mutations in yeast by gap repair for further testing and validation. The results of the mutagenesis screen are provided in Table 6. Residues confirmed to positively impact reticuline titer (and hence norcoclaurine synthase activity) were then subjected to saturation mutagenesis. Specifically, oligos were ordered, each with an NNN codon at the position of the residue of interest. The new PCR products were transformed into YA139 to generate a library of NCS variants with every possible codon at the target residue. These new saturation mutagenesis libraries were then screened for enhanced reticuline titers. The results of the saturation mutagenesis screen are provided in Table 7. Finally, improved variants from the random mutagenesis and saturation mutagenesis screens were shuffled to identify combinations of mutations that gave the greatest increase in NCS activity. The results of the shuffling screen are provided in Table 8. The random mutagenesis, saturation mutagenesis, and shuffling rounds were later repeated with NCS2 (SEQ ID NO: 72) as a template.

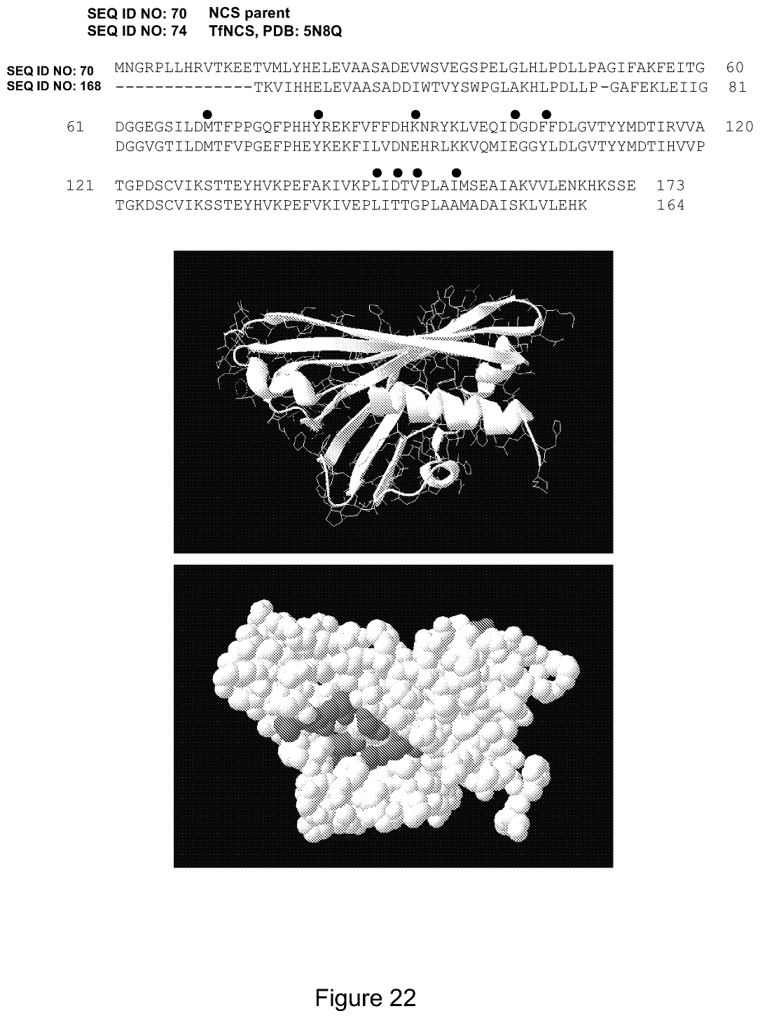

The residues identified as being important for catalytic function in the mutagenesis screen included amino acids 70, 81, 91, 101, 104, 147, 149, 151, and 155 of NCS parent, SEQ ID NO: 70. To identify the structural locations important for engineering Bet v I enzymes, NCS parent (SEQ ID NO: 70) was aligned with TfNCS (PDB: 5N8Q) (SEQ ID NO: 74), a natural norcoclaurine synthase enzyme with a solved crystal structure. Mapping of the residues from NCS parent to TfNCS (PDB: 5N8Q) revealed that residues M70, Y81, D101, F104, L147, D149, V151, I155 are located at the entry point to the active site (FIG. 22). One exception is K91 which is located at the opposite end of the catalytic tunnel.

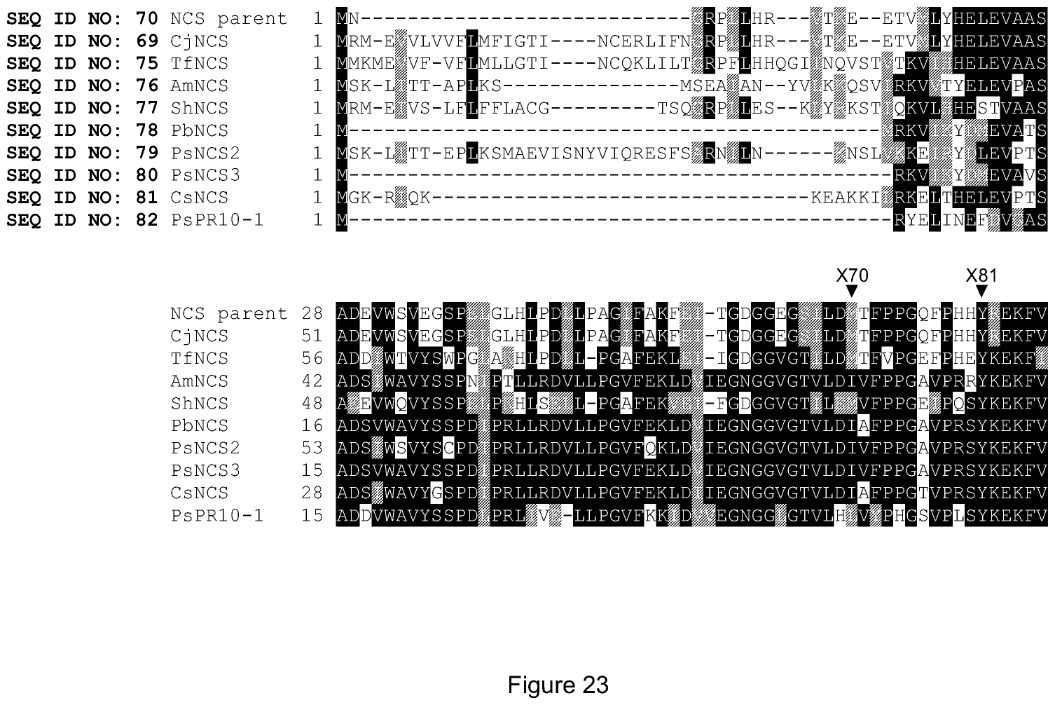

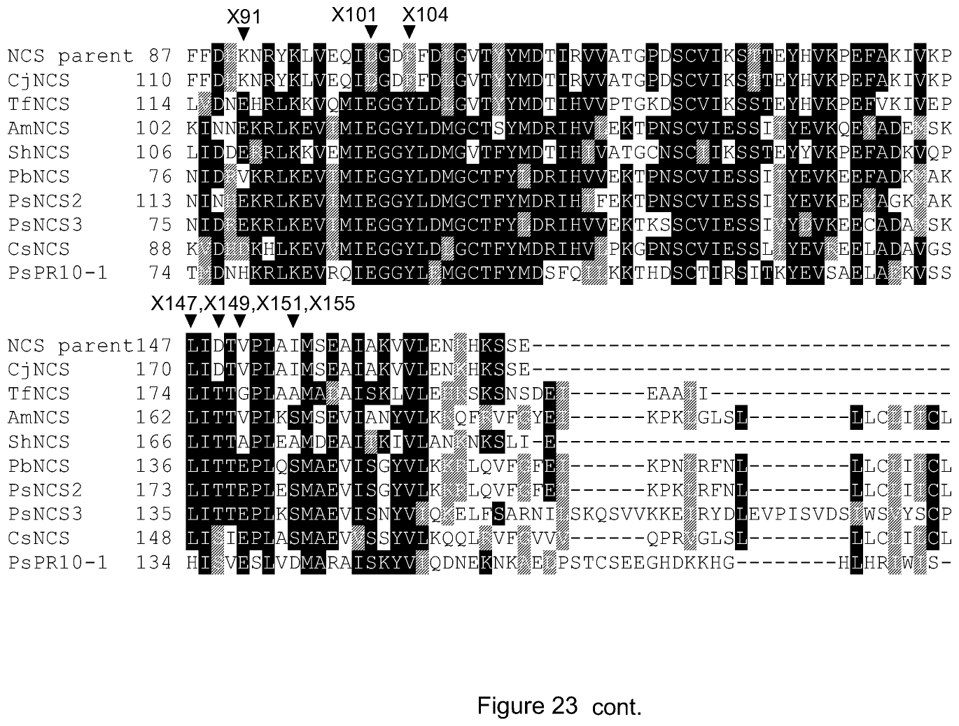

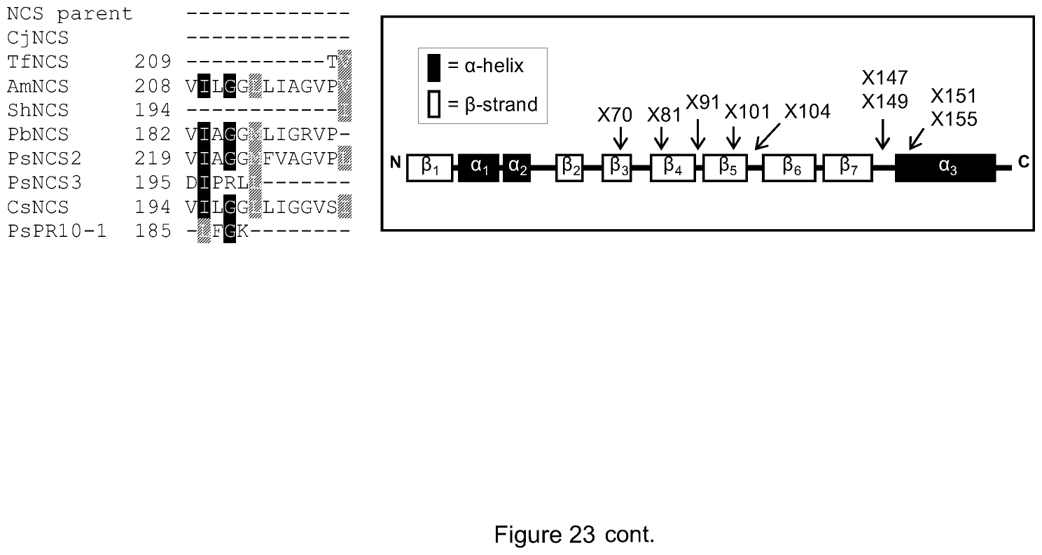

To further characterize the structural features of Bet v I enzymes that are important for engineering improved activity, NCS parent (SEQ ID NO: 70) was aligned with plant Bet v I proteins from Coptis japonica, Thalictrum flavum, Argemone mexicana, Sinopodophyllum hexandrum, Papaver bracteatum, Papaver somniferum, and. Cordalyis saxicola (FIG. 23). Given that Bet v I proteins are highly conserved at the structural level, the indicated residues of the NCS parent sequence point to the equivalent structural and sequence locations for engineering any one of these or other Bet v I proteins. The locations in the alpha helices and beta strands common to plant Bet v I proteins are shown in the inset of FIG. 23 and described in Table 9.

In general, the norcoclaurine synthase natural and improved variants are described and recorded here as amino acid sequences using the standard one letter abbreviation for amino acid residues. These peptide sequences could be translated to nucleotide sequences using any of the codons in Table 10. In addition, any one amino acid could be substituted for another amino acid of similar properties as indicated in Table 10.

TABLE 6
Results of a random mutagenesis screen
to identify improved variants of NCS.

| Base pair | | Amino acid | |
|---|---|---|---|
| mutation | Codon change | change | |
| | | | Activity (relative |
| | | | to SEQ ID NO: 70) |
| A → T | TAC → TTC | Y81F | 1.3 |
| A → G | AAG → GAG | K91E | 1.2 |
| T → A | GAT → GAA | D101E | 1.9 |
| T → C | TTC → CTC | F104L | 1.3 |
| T → A | TTG → ATG | L147M | 1.1 |
| G → A | GAT → AAT | D149N | 1.5 |
| T → A | GTT → GAT | V151D | 1.1 |
| T → A | ATT → AAT | I155N | 1.5 |
| | | | Activity (relative |
| | | | to SEQ ID NO: 72) |
| G → A | ATG → ATA | M70I | 1.6 |

TABLE 7
Results of an NNN saturation mutagenesis
screen at individual residues of NCS.

| | | Mutant: | | |
|---|---|---|---|---|
| Parent: | | | | Activity |
| | Parent | Amino acid | New | (relative to |
| Residue | codon | substitution | codon | SEQ ID NO: 70) |
| M70 | ATG | L | CTA | 1.5 |
| | | L | TTG | 1.7 |
| | | I | ATT | 1.5 |
| | | I | ATC | 1.5 |
| | | V | GTC | 1.4 |
| Y81 | TAC | F | TTT | 1.6 |
| K91 | AAG | A | GCG | 1.6 |
| | | A | GCC | 1.5 |
| | | V | GTT | 1.5 |
| | | V | GTC | 1.6 |
| | | S | AGT | 1.5 |
| | | S | TCA | 1.6 |
| | | L | CTT | 1.6 |
| D101 | GAT | S | AGT | 1.1 |
| | | S | TCA | 1.2 |
| | | S | AGC | 1.1 |
| | | A | GCA | 1.1 |
| F104 | TTC | L | TTG | 1.5 |
| L147 | TTG | Y | TAC | 1.5 |
| | | Y | TAT | 1.3 |
| | | H | CAC | 1.3 |
| | | F | TTC | 1.4 |
| D149 | GAT | N | AAT | 1.3 |
| | | T | ACG | 1.3 |
| | | T | ACA | 1.3 |
| | | T | ACC | 1.5 |
| | | S | AGT | 1.2 |
| V151 | GTT | E | GAG | 1.6 |
| | | A | GCA | 1.6 |
| | | A | GCG | 1.7 |
| | | A | GCT | 1.6 |
| | | K | AAG | 1.7 |
| | | K | AAA | 1.6 |
| | | T | ACG | 1.7 |
| | | S | AGT | 1.7 |
| I155 | ATT | Q | CAG | 2.1 |
| | | A | GCT | 1.4 |
| | | T | ACG | 1.5 |
| | | S | AGT | 1.4 |
| | | N | AAT | 1.4 |

TABLE 8
Shuffled variants of NCS.

| Mutations (relative to SEQ ID NO: 70) | |
|---|---|
| | Activity (relative to |
| | SEQ ID NO: 70) |
| D149T, I155Q | 2.5 |
| D149T, I155N | 2.8 |
| F104L, D149N, I155Q | 2.7 |
| K91S, D149T, I155Q | 2.4 |
| D149S, I155Q | 2.5 |
| K91A, D149T, I155Q | 2.3 |
| K91A, D149T, I155Q | 2.7 |
| K91S, D101S, F104L, D149T, I155Q | 2.5 |
| K91V, D149S, I155N | 2.4 |
| Y81F, K91A, D149N | 2.3 |
| Y81F, D149N, I155N | 2.4 |
| | Activity (relative to |
| | SEQ ID NO: 5) |
| N2S, M70L, F140A, D149T, I155N | 1.1 |
| M70L, K91A, D149T, I155N | 1.1 |

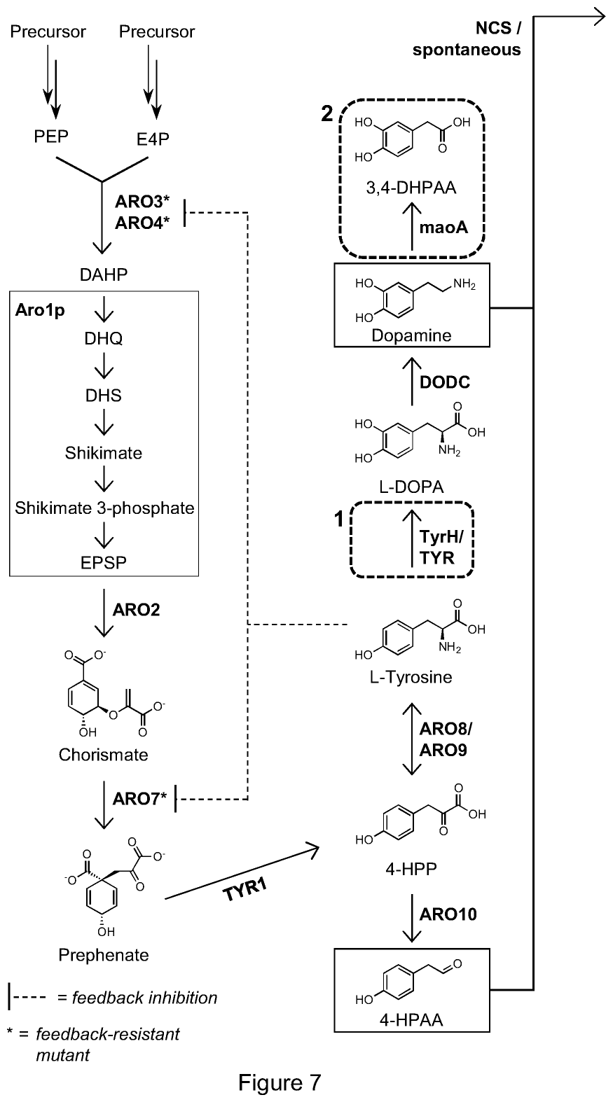

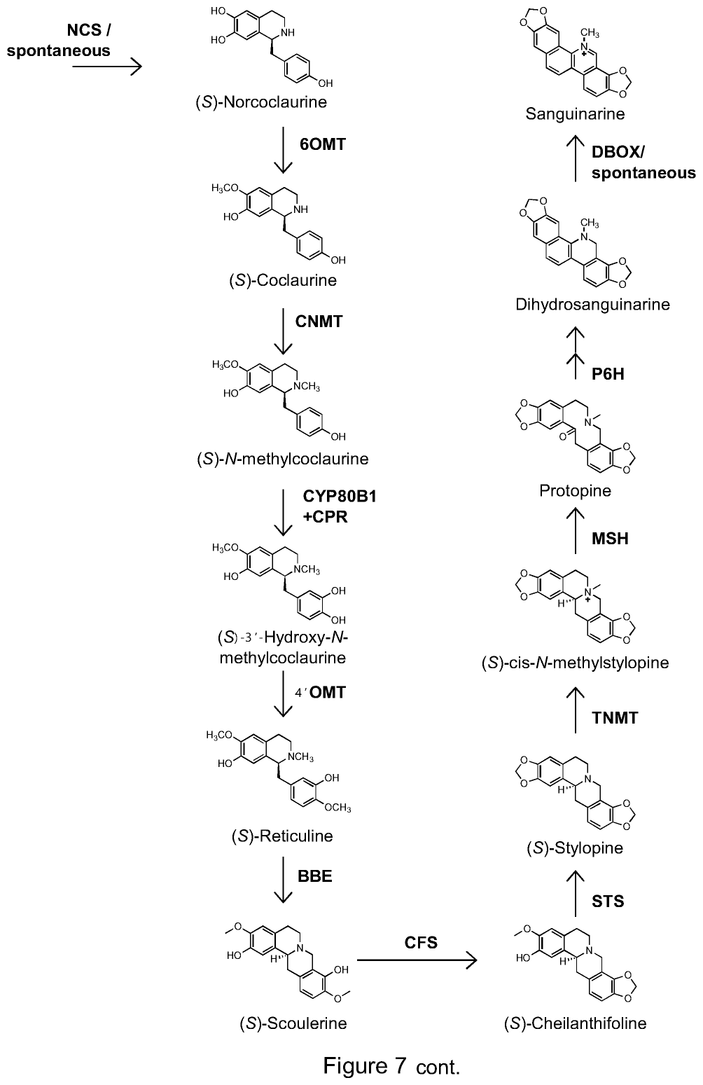

TABLE 9
Structural locations useful for engineering Bet v I fold enzymes.

| Corresponding position in | Structural location |
|---|---|
| SEQ ID NO: 70 | (see FIG. 7) |
| X70 | Beta strand 3 |
| X81 | Beta strand 4 |
| X91 | Between beta strands 4 and 5 |
| X101 | Beta strand 5 |
| X104 | Between beta strands 5 and 6 |
| X147 | Between beta strand 7 and alpha helix 3 |
| X149 | Between beta strand 7 and alpha helix 3 |
| X151 | Alpha helix 3 |
| X155 | Alpha helix 3 |

TABLE 10
Amino acid (also called “residue”) abbreviations, codons, and possible
substitutions for amino acids with similar side chains and properties.

| Amino acid/ | One letter | Three letter | | |
|---|---|---|---|---|
| residue | abbreviation | abbreviation | Possible codons | Possible substitutions |
| Alanine | A | Ala | GCT, GCC, GCA, GCG | Other aliphatic, non-polar: |
| | | | | V, I, L, M, G |
| Arginine | R | Arg | CGT, CGC, CGA, CGG, | Other basic polar: H, K |
| | | | AGA, AGG | |
| Asparagine | N | Asn | AAT, AAC | Other uncharged polar: S, |
| | | | | T, Q |
| Aspartic acid | D | Asp | GAT, GAC | Other acidic polar: E |
| Cysteine | C | Cys | TGT, TGC | |
| Glutamic acid | E | Glu | GAA, GAG | Other acidic polar: D |
| Glutamine | Q | Gln | CAA, CAG | Other uncharged polar: S, |
| | | | | T, N |
| Glycine | G | Gly | GGT, GGC, GGA, GGG | Other aliphatic, non-polar: |
| | | | | A, V, I, L, M |
| Histidine | H | His | CAT, CAC | Other basic polar: R, K |
| Isoleucine | I | Ile | ATT, ATC, ATA, | Other aliphatic, non-polar: |
| | | | | A, V, L, M, G |
| Leucine | L | Leu | TTA, TTG, CTT, CTC, | Other aliphatic, non-polar: |
| | | | CTA, CTG | A, V, I, M, G |
| Lysine | K | Lys | AAA, AAG | Other basic polar: R, H |
| Methionine | M | Met | ATG | Other non-polar: A, V, I, |
| | | | | L, G |
| Phenylalanine | F | Phe | TTT, TTC | Other aromatic: Y, W |
| Proline | P | Pro | CCT, CCC, CCA, CCG, | |
| Serine | S | Ser | TCT, TCC, TCA, TCG, | Other uncharged polar: N, |
| | | | AGT, AGC | T, Q |
| Threonine | T | Thr | ACT, ACC, ACA, ACG | Other uncharged polar: S, |
| | | | | N, Q |
| Tryptophan | W | Trp | TGG | Other aromatic: Y, F |
| Tyrosine | Y | Tyr | TAT, TAC | Other aromatic: F, W |
| Valine | V | Val | GTT, GTC, GTA, GTG, | Other aliphatic, non-polar: |
| | | | | A, I, L, M, G |

### Example 13: Production of BIAs in Microbial Strains with Engineered NCS

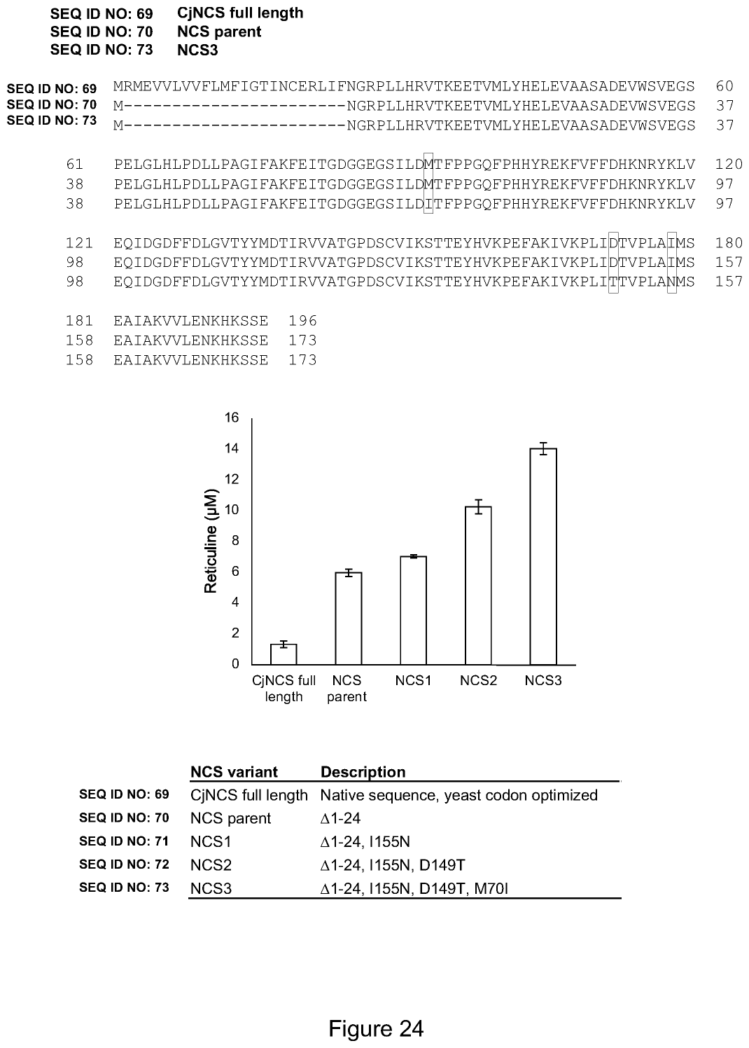

To demonstrate the utilization of the engineered NCS enzymes in enhancing the production of benzylisoquinoline alkaloids in yeast, both untruncated and truncated NCS variants with one, two, or three beneficial mutations were used to generate an enzyme ladder. Specifically, plasmids encoding CjNCS full length (SEQ ID NO: 69), NCS parent (SEQ ID NO: 70), NCS 1 (SEQ ID NO: 71), NCS2 (SEQ ID NO: 72), and NCS3 (SEQ ID NO: 73) were transformed into YA139. When cultured under standard assay conditions in 96-well microtiter plates (and without tyrosine supplementation), NCS 3 with a 24 amino acid N-terminal truncation and mutations M70I, D149T, and I155N, produced 14.02 μM reticuline (FIG. 24). This observed titer was more than 10-fold greater than the 1.31 μM reticuline produced by a strain expressing CjNCS full length.

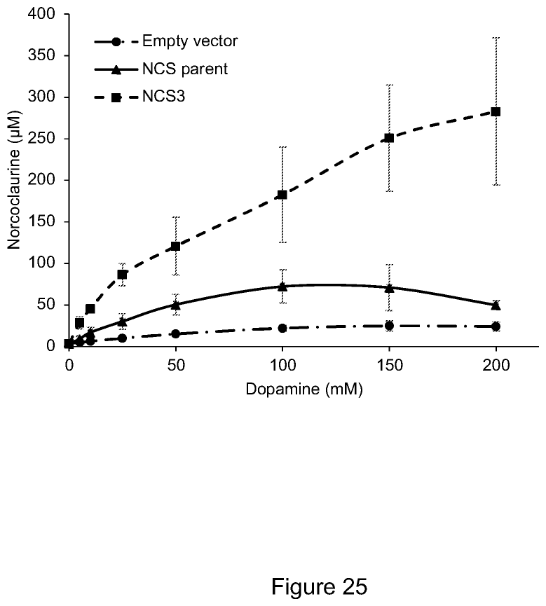

To further characterize the enhanced activity of NCS3 (SEQ ID NO: 73), a wild type CEN.YPK2 strain was transformed with plasmids carrying either NCS parent (SEQ ID NO: 70), NCS3 (SEQ ID NO: 73), or an empty plasmid (no-enzyme control). Strains were cultured in 96-well microtiter plates with medium supplemented with 300 mg/L tyrosine (to promote 4-HPAA production by the host cell's native metabolism) and 0, 5, 10, 25, 50, 100, 150, 200 mM dopamine. After 48 hours growth the plates were centrifuged to pellet the cells and the final norcoclaurine titer determined for each well by LCMS (FIG. 25). The strain expressing NCS parent produced 72.1 μM norcoclaurine when supplied with 100 mM dopamine in the culture medium. However, at higher concentrations of 150 and 200 mM dopamine, the norcoclaurine yield for this strain decreased, indicating the NCS parent suffered inhibition at dopamine concentrations over 100 mM. In contrast, the strain expressing NCS3 accumulated 182.6 μM norcoclaurine at 100 mM dopamine, and titers increased further to 250.8 and 283.0 μM norcoclaurine at dopamine supplementation of 150 and 200 mM, respectively.

### Example 14: Production of Thebaine Using Engineered Microbial Strains

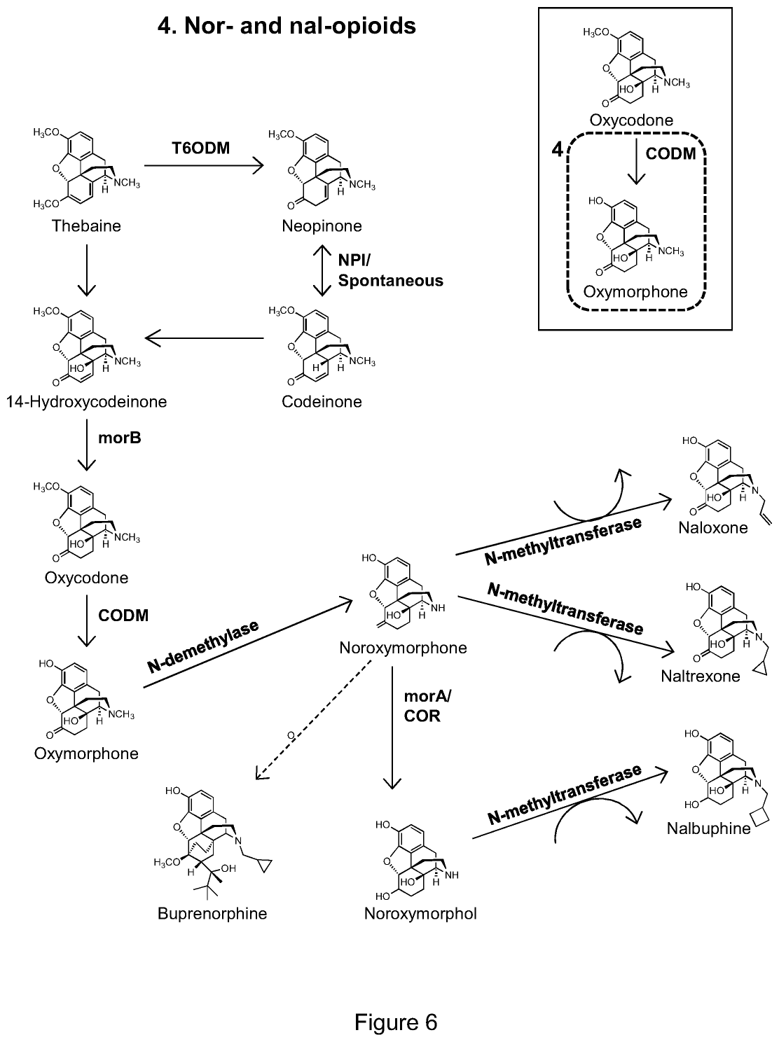

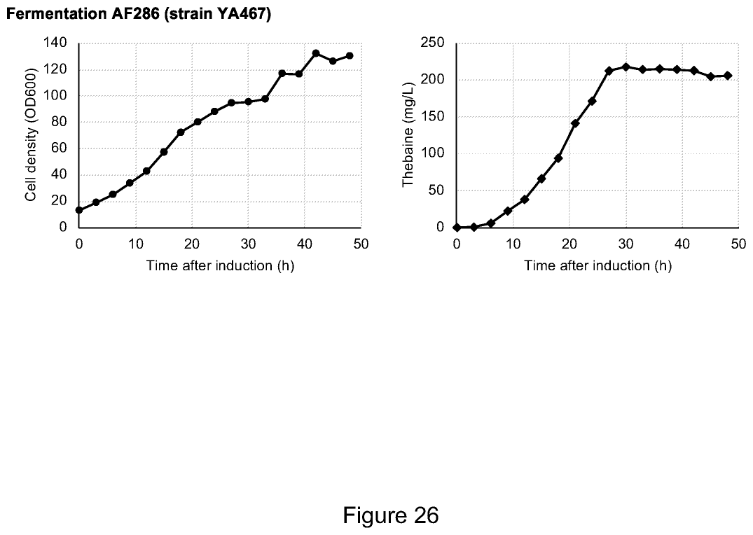

A thebaine production stain was constructed that included three copies of the improved norcoclaurine synthase variant, NCS3, and the complete biosynthetic pathway to thebaine (strain YA467, FIG. 4). Strain YA467 was cultured in a 1 L fermentor with fed-batch glucose (fermentation AF286). The synthetic complete medium was further supplemented with amino acids, vitamins, salts, and trace elements at the start of the culture and at time points during the fermentation. After 48 h the bioprocess reached OD₆₀₀ 130 and achieved a final titer of 206 mg/L thebaine (FIG. 26). The thebaine strain and bioprocess could be further modified to produce any of the opioid molecules depicted in FIGS. 4 and 6.

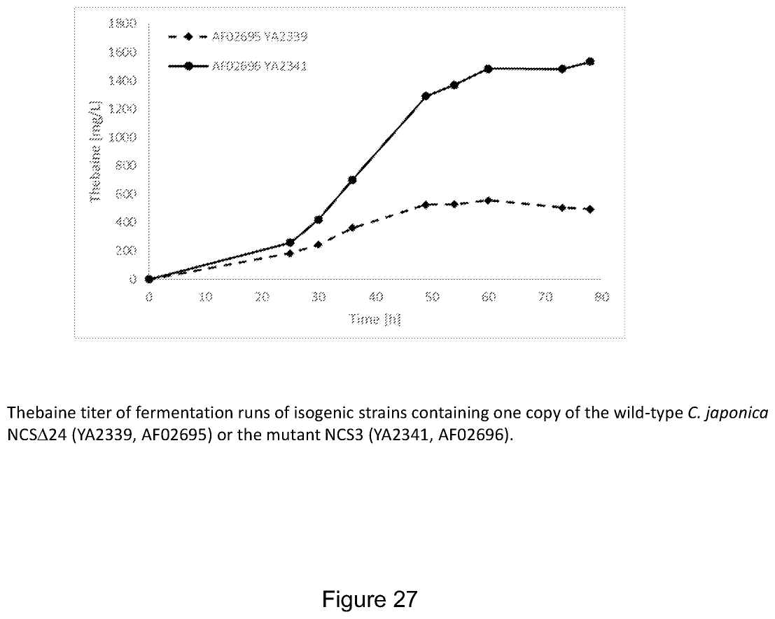

The improved variant NCS3 was also compared to the wild-type NCSA24 sequence in strains producing thebaine from glucose. Strains were run in a fed-batch fermentation process supplemented with additional amino acids, vitamins, salts, and trace elements. The strain with NCS3 (YA2341, AF02696) produced 1.5 g/L thebaine compared to the wild-type sequence with an N-terminal truncation (YA2339, AF02695) that produced only 0.5 g/L thebaine after 78 h (FIG. 27).

### Example 15: Platform Microbial Strains Engineered to Produce Thebaine with Reduced Ethanol and Fusel Alcohol Production

Yeast strains that produce thebaine and use 4HPAAS can be further engineered to reduce ethanol and fusel alcohol accumulation. The platform yeast producing thebaine from glucose and using 4HPAAS was further engineered to reduce production of acetaldehydes by disruption of pyruvate decarboxylase genes PDC1, PDC5, and PDC6. Without pyruvate decarboxylase activity, an alternative route to acetyl-CoA is required for growth on glucose. An expression cassette was constructed (P_{TDH3}-CaPK) to introduce a phosphoketolase enzyme that converts fructose-6-phosphate to acetyl-P and erythrose-4-phosphate. Acetyl-P is spontaneously converted to acetate which can then go to acetyl-CoA using the native acetyl-CoA synthetases (ACS1 or ACS2). Alternatively, a phosphotransacetylase enzyme (PTA) can be used to convert acetyl-P directly to acetyl-CoA which uses less ATP. With an alternative route to acetyl-CoA in place, the pyruvate decarboxylase genes PDC1, PDC5, and PDC6 were disrupted.

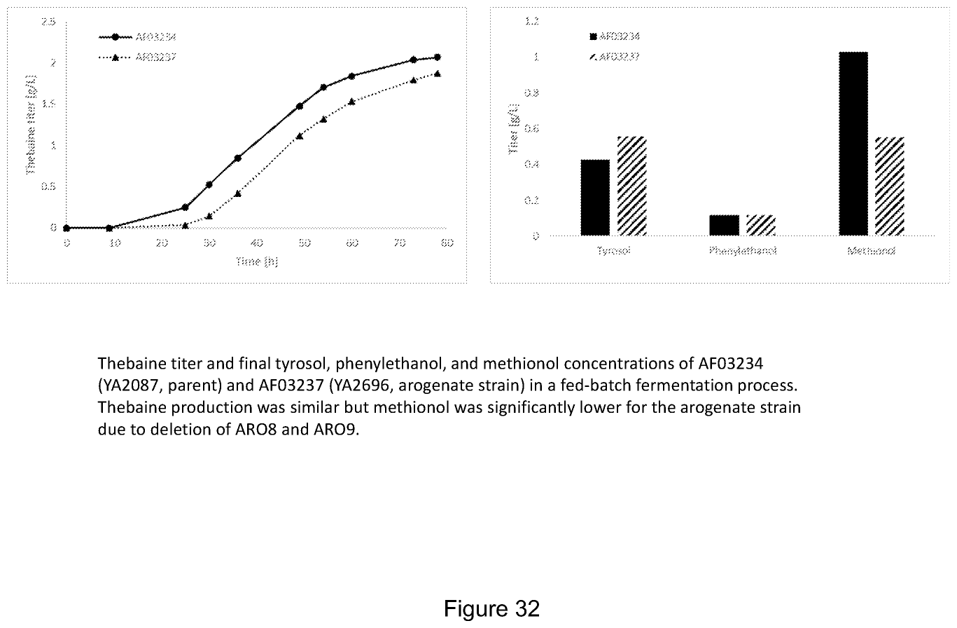

The resulting strain YA2156 with no PDC activity and PK integrated was compared to parent YA2087 in a shake flask experiment. After overnight growth in minimal media, strains were diluted 1 to 100 in fresh media containing 8% maltodextrin plus amylase to provide a slow release of glucose along with additional vitamins and amino acids. Shake flask cultures were incubated with shaking at 30° C. for 72 hours. Thebaine titer for YA2156 was 0.17 g/L and titer for YA2087 was 0.55 g/L (FIG. 32). In YA2156, tyrosol was not detected, phenylethanol was 0.11 g/L, and methionol was 0.25 g/L. In YA2087, tyrosol was 0.05 g/L, phenylethanol was 0.12 g/L, and methionol was 0.42 g/L. Neither strain accumulated significant ethanol under this condition.

### Example 16: Platform Microbial Strains Engineered to Produce Thebaine with Increased SAM and Improved Methyltransferase Activity

Yeast strains that produce thebaine can be further engineered to increase the co-factor S-adenosyl-L-methionine (SAM) and improve the flux through several methyltransferase enzymes in the pathway. In a platform yeast strain producing thebaine, additional constructs were integrated to add two additional copies of SAH1 and one copy of SAM2 from S. cerevisiae (P_{TDH3}-SAH1 at the PAU24 locus and SAM2-P_{GAL1,10}-SAH1 at the BAS1 locus).

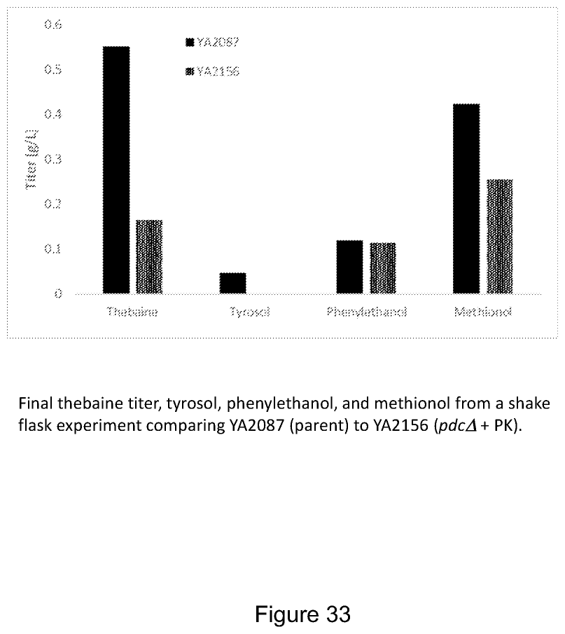

The resulting strain YA1669 with additional copies of SAM2 and SAH1 was run in a 1 L fermentor in a glucose fed-batch process (fermentation AF01877). The medium was supplemented with vitamins, trace elements, and amino acids. After 78 h, the thebaine titer of YA1669 was 2.4 g/L compared to only 1.3 g/L for the parent strain (YA1158, AF01863) (FIG. 33). The methyltransferase substrates norcoclaurine, coclaurine, and 3′OH—N-methylcoclaurine were significantly reduced.

### Example 17: Platform Microbial Strains Engineered to Produce Thebaine that Converts Aromatic Fusel Alcohols to Less Toxic by-Products

Yeast strains that produce thebaine can be further engineered to convert excess fusel alcohols tyrosol and/or phenylethanol to salidroside and phenylethyl beta-D-glucoside. In a platform yeast strain producing thebaine, an additional constructs were integrated to express the UGT33 enzyme from Rhodiola rosea (P_{TDH3}-UGT33 at the HXT5 locus and P_{GAL7}-UGT33 deleting the EGH1 locus). A third copy of UGT from Oryza sativa was integrated (P_{TDH3}-UGT45) at the HST2 locus and BAT2 was also deleted in this background.

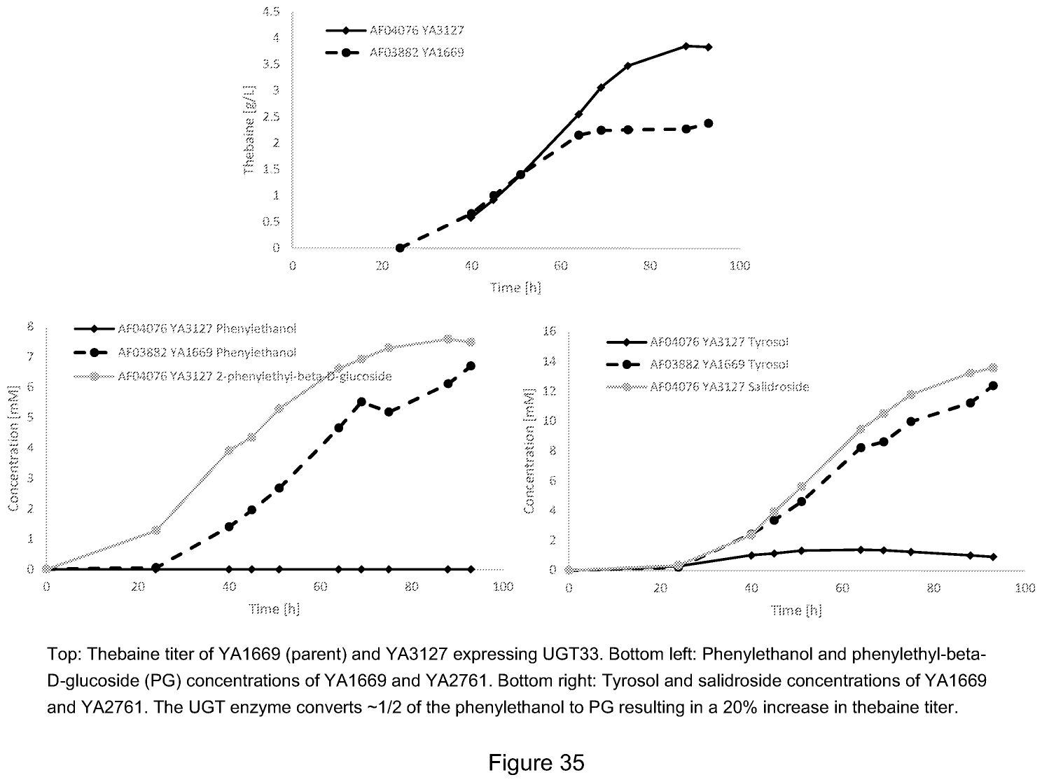

The resulting strain YA3127 producing thebaine and also expressing UGT33 was run in a 1 L fermentor in a glucose fed-batch process (fermentation AF04076). YA3127 produced 3.8 g/L thebaine compared to the parent strain YA1669 which made 2.4 g/L thebaine (FIG. 35). The UGT preferentially used phenylethanol as a substrate and converted nearly all of the phenylethanol to the glucoside (PG). Over 80% of the tyrosol was also converted to salidroside, resulting in a 65% increase in thebaine titer.

### Example 18: Microbial Strains Engineered to Reduce Formaldehyde Toxicity and Maintain Glutathione Pool

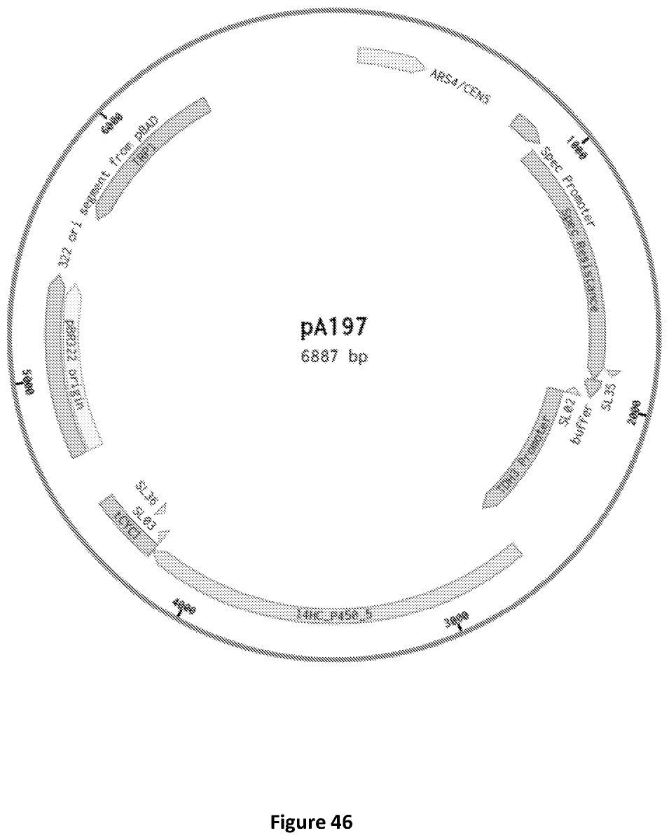

Yeast strains were engineered according to the present disclosure to support the production of benzylisoquinoline alkaloids from thebaine by yeast fermentation. In this Example, yeast strains were engineered to support the production of codeine and codeinone; however, the stains could be used to engineer alternative benzylisoquinoline alkaloids (e.g., morphine). Specifically, strains were engineered to reduce formaldehyde toxicity and maintain the glutathione pool in the cell. To produce noroxymorphone, a yeast strain must also produce morphinan intermediates, particularly benzylisoquinoline alkaloids (BIAs) such as codeinone, codeine, hydrocodone, morphinone, and/or others. A key step in the production of morphinan intermediates is oxidation of an intermediate methylated at position 6 (e.g., thebaine, oripavine, northebaine, nororipavine and the like). This demethylation can be catalyzed by the 20DD enzyme, T6ODM, or other suitable oxidase with similar activity. The product of this reaction depends on the substrate used, and potential products include, for example, neopinone, neomorphinone, norneopinone (N-demethylate neopinone), norneomorphinone (N-demethylated neomorphinone) or the like. Regardless of the oxidative enzyme used, the substrate, or product, oxidation of a methyl group for demethylation produces formaldehyde as a byproduct. An exemplary reaction illustrating this demethylation during codeine biosynthesis is shown in FIG. 46. Formaldehyde is a necessary byproduct of this reaction, but it is undesirable because formaldehyde is toxic to yeast.

The major formaldehyde detoxification pathway in yeast utilizes the formaldehyde dehydrogenase enzyme SFA1. (Achkor, H., Diaz, M., Fernindez, M. R., Biosca, J. A., Pards, X., & Martinez, M. C. (2003). Enhanced formaldehyde detoxification by overexpression of glutathione-dependent formaldehyde dehydrogenase from Arabidopsis. Plant Physiology, 132(4), 2248-2255). This pathway is depicted in FIG. 37. In this pathway, formaldehyde spontaneously conjugates to glutathione, a common yeast metabolite, to form S-hydroxymethyglutathione. SFA1 then oxidizes S-hydroxymethyglutathione to S-formylglutathione.

By increasing flux to this formaldehyde detoxification pathway, there is a risk that glutathione is depleted inside the cell. Glutathione is necessary for formaldehyde detoxification via SFA1. Accordingly, it is desirable to maintain the glutathione pool to allow for continued formaldehyde detoxification. Proteins DUG2 and DUG3 together form a peptidase complex which cleaves the link between glutamate and cysteine (Baudouin-Cornu, P., Lagniel, G., Kumar, C., Huang, M. E., & Labarre, J. (2012). Glutathione degradation is a key determinant of glutathione homeostasis. Journal of Biological Chemistry, 287(7), 4552-4561.). By preventing cleavage of the link between glutamate and cystine, glutathione degradation can be reduced thereby preserving the glutathione pool in the cell. Both DUG2 and DUG3 are necessary for formation of the active peptidase in yeast. Accordingly, removing DUG2 and/or DUG3 prevents expression of the active enzyme.

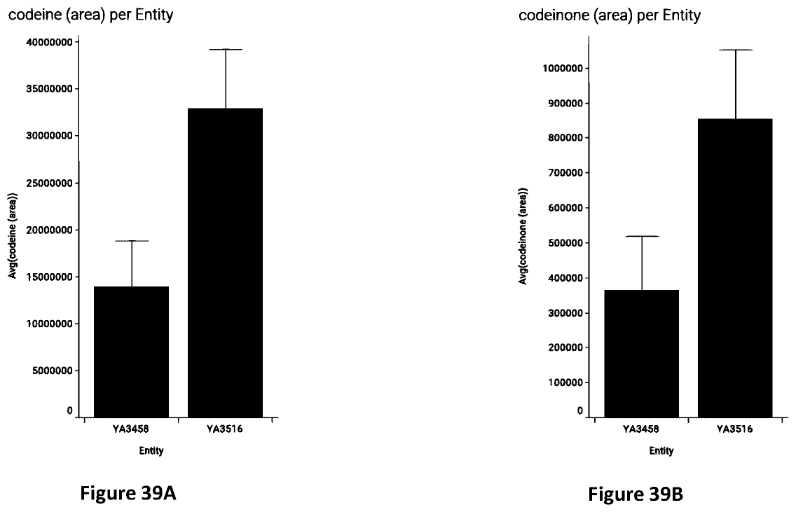

The yeast strain in this Example was engineered to increase overall codeine production by removing the formaldehyde that is formed by T6ODM from the strain. This was achieved by converting the formaldehyde into a less toxic metabolite using a formaldehyde dehydrogenase. Specifically, an engineered Saccharomyces cerevisiae strain producing codeine but auxotrophic for the SFA1 enzyme (YA3458) was further engineered to constitutively overexpress SFA1 by placing a wildtype SFA1 sequence under the control of a constitutive promoter that was integrated into the yeast chromosome. Because glutathione—which is necessary for formaldehyde detoxification via SFA1—can be depleted by catabolism to glutamate and cysteine during the formaldehyde detoxification process (FIG. 36), the strain in this Example was further engineered to maintain the glutathione pool by preventing DUG2/3 from catabolizing glutathione into glutamate and cysteinylglycine. Specifically, the YA3458 strain (which is wildtype for DUG3) was further engineered to knock out DUG3 by replacing it with the constitutively-overexpressed SFA1. To support the removal of formaldehyde from cells, the YA3458 Saccharomyces cerevisiae strain was engineered to constitutively overexpress the enzyme SFA1 by placing a wildtype SFA1 sequence under the control of a constitutive overexpression promoter that was integrated into the yeast chromosome. The resultant strain (YA3516) with the DUG3\hat{ }::SFA1 engineering of the present Example makes 2.5 times as much codeinone and codeine compared to the control strain YA3458, which has an active DUG3 but no SFA1 (FIGS. 39A, 39B).

### Example 19: Microbial Strains Engineered to Produce 14-Hydroxylated Opioid Compounds from Thebaine

Platform yeast strains can be engineered to produce the morphinan alkaloid thebaine de novo in high titers as described in Example 18. Yeast strains that produce thebaine can be further engineered to produce 14-hydroxylated opioid compounds from thebaine, including noroxymorphone. One of the enzymes required for this pathway is a 14-hydroxylase, an enzyme capable of installing a hydroxyl group on the 14-position of one of the intermediates between thebaine and noroxymorphone. There are numerous intermediates between thebaine and noroxymorphone, including, for example, codeinone, norcodeinone, hydrocodone, northebaine, oripavine, morphinone, normorphinone, hydromorphine, norhydromorphine, norhydrocodone, and codeine. The 14-hydroxylation can occur on any intermediate between thebaine and noroxymorphone because enzymes in this pathway are capable of acting on multiple substrates, and the final product (noroxymorphone) must possess such hydroxyl group in the 14-position. Certain organisms have been reported to produce 14-hydroxylated compounds.

One class of enzymes capable of performing the 14-hydroxylation of an intermediate between thebaine and noroxymorphone are the cytochromes P450 (P450s). As discussed above, P450s are monooxygenases which incorporate a hydroxyl group onto diverse substrates at diverse positions, but P450s often demonstrate remarkable substrate- and regio-selectivity (i.e., can specifically target only one or a select few of many available positions). Several P450s have been reported to accept benzylisoquinoline alkaloids (BIAs) as substrates, which is the class of molecules to which thebaine, noroxymorphone, and intermediates between the two belong. See, e.g., International Patent Publication No. WO2022/109194A1. None have been reported, to our knowledge, to accomplish the 14-hydroxylation of any BIA substrate. P450s that are active on BIA substrates were examined to determine whether they are capable of performing a 14-hydroxylation reaction on intermediates between thebaine and noroxymorphone.

Codeine was selected as the first target substrate because we have engineered yeast strains that produce high titers of codeine de novo as described in Example 19, codeine and 14-hydroxycodeine are stable molecules, codeine is a potential intermediate between thebaine and noroxymorphone, and codeine has chemical similarities to several other potential intermediates. Exemplary P450s that have been shown to accept at least one BIA as a substrate are listed in Table 17. Each P450 listed in Table 17 (14HC_P450_1-32) was individually incorporated using a vector into an engineered yeast strain that produces codeine. See FIG. 39. An exemplary vector for incorporating a P450 into a microbial strain is depicted for 14HC_P450_5 in FIG. 46 (“pA197”; sequence provided in Table 17).

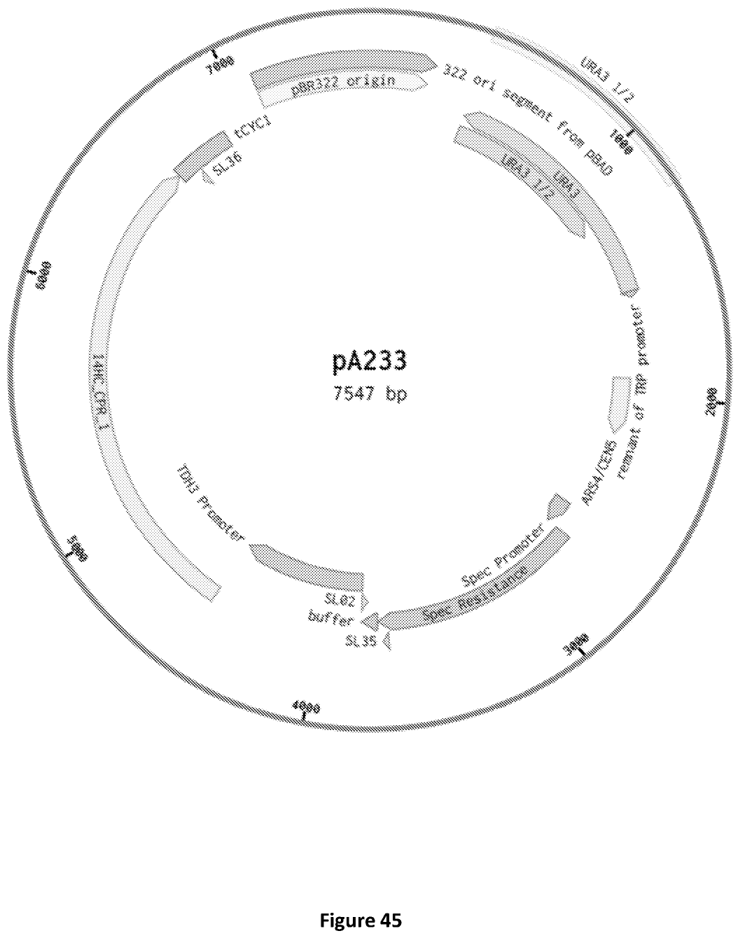

P450s generally require a partner enzyme to function (a cytochrome P450 reductase, or “CPR”). CPRs provide the necessary reducing power so a P450 can reduce oxygen to produce an active intermediate for hydroxylation. The microbial strains were therefore further engineered to express a CPR listed in Table 18 (14HC_CPR_1-4) in addition to a P450 as described below. An exemplary vector for incorporating a CPR into a microbial strain is depicted for 14HC_CPR_1 in FIG. 45 (“pA233”; sequence provided in Table 19).

Specifically, genes encoding CPR and C-14-hydroxylase candidates were codon optimized for S. cerevisiae expression and synthesized (named 14HC_CPR1-3 and 14HC_P450_1-32, respectively). Genes were first amplified via PCR and cloned into a yeast expression vector via Gibson assembly (FIGS. 45 and 46; Table 19). CPR candidates were cloned into pA48 (ARS/CEN origin of replication, uracil marker, TDH3 promoter and CYC1 terminator), and 14-hydroxylase candidates were cloned into pA36 (ARS/CEN origin of replication, tryptophan marker, TDH3 promoter and CYC1 terminator). A person of skill in the art will understand that the CPR and C-14 hydroxylase candidates may be expressed using any suitable platform in the host cell, including, but not limited to, using a different plasmid, yeast artificial chromosome, or genome.

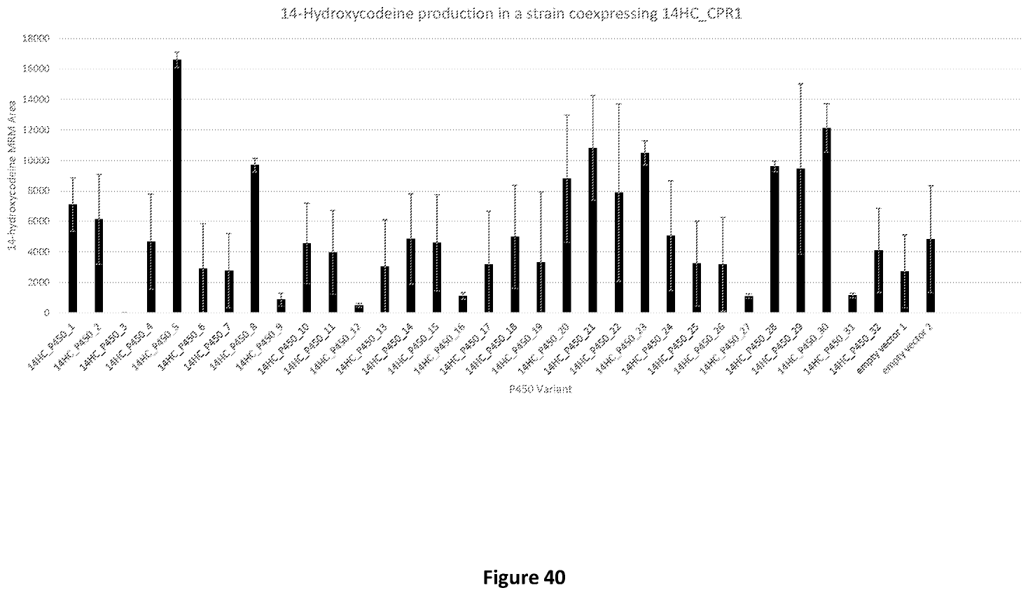

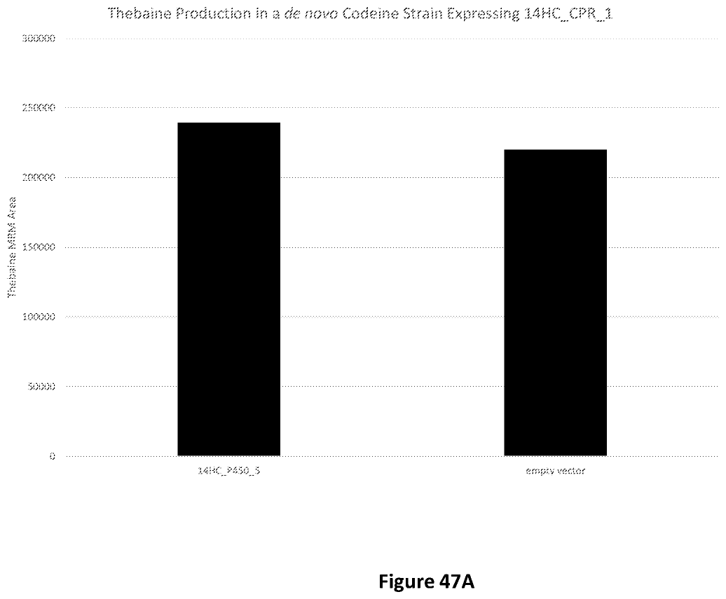

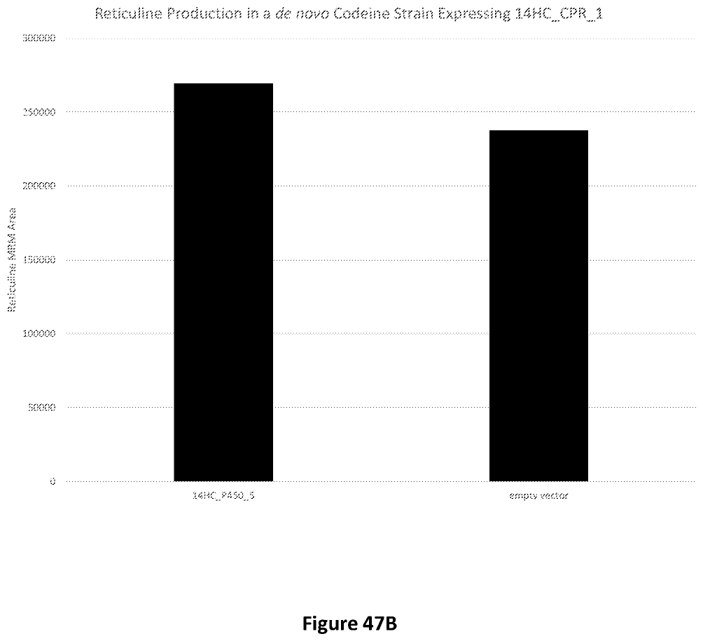

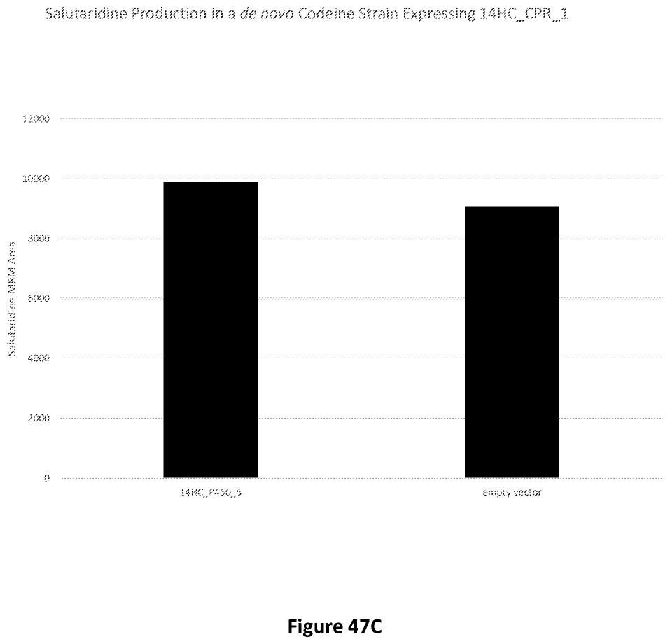

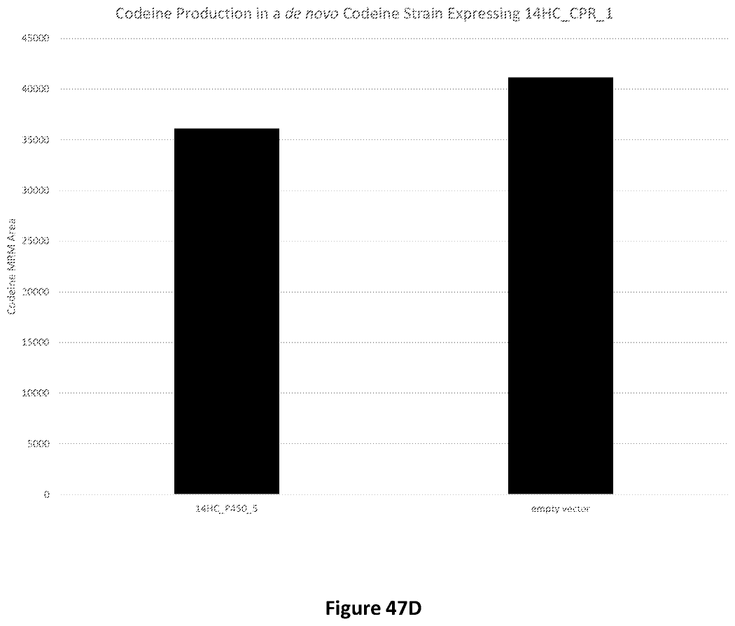

To examine 14-hydroxycodeine production by strains expressing different C-14 hydroxylase candidates, plasmids encoding a CPR candidate (14HC_CPR1) were transformed in tandem with a plasmid encoding a C-14 hydroxylase candidate (14HC_P450_1-32) into yeast strain YA3509 (a codeine producing strain engineered as described in Example 18 that was further engineered to be auxotrophic for tryptophan and uracil) using a commercially available transformation kit and were plated on selective solid media. A person of ordinary skill in the art will understand that other suitable yeast transformation protocols may be used. After 3 days of culturing at 30° C., colonies randomly selected and cultured in 400 μL of standard synthetic complete (SC) dropout liquid medium for 72 hours at 28° C. in a 96-well plate. Then, 20 μL of each culture was transferred to 380 μL of fresh production media at pH 3.65 in a 96-well plate in replicates of 3 and grown for an additional 72 hours at 28° C. The production media in the present Example was prepared containing 3.13 g/L ammonium sulfate, 5 g/L monosodium glutamate, 2.75 g/L methionine, 0.03 g/L inositol, 0.1 g/L ampicillin, 19.2 g/L citric acid, 0.014 g/L EDTA, 0.55 mg/L copper(II)sulfate, 0.25 mg/L iron(III) chloride, 3.0 mg/L iron(III)sulfate heptahydrate, 3.0 mg/L manganese(II)sulfate monohydrate, 0.75 mg/L sodium molybdate(VI) dihydrate, 1.4 mg/L zinc sulfate heptahydrate, 3.0 mg/L boric acid, 0.6 mg/L potassium iodide, 0.25 g/L magnesium sulfate heptahydrate, 0.63 g/L potassium phosphate, 0.63 mg/L biotin, 1.5 mg/L p-aminobenzoic acid, 3.0 mg/L nicotinic acid, 8.1 mg/L pyridoxine hydrochloride, 3.0 mg/L thiamine hydrochloride, 13 mg/L D-calcium pantothenate, 14 mg/L adenine sulfate, 80 g/L maltodextrin and 2 units/L amylase. The amylase is used to mimic a fed-batch process and gradually releases maltose from maltodextrin polymer so that the yeast can use it as a carbon source. Following the 72-hour incubation, the cells were separated from the media by centrifugation and the supernatant was diluted 10× in water with 0.1% formic acid. 14-hydroxycodeine in the supernatant was measured by LC-MS/MS analysis (FIG. 40). Further LC-MS/MS analysis was performed on an exemplary engineered 14-hydroxycodeine strain expressing a CPR (14HC_CPR_1) and a 14-hydroxylase (14HC_P450_5) or an empty vector control as described. LC-MS/MS revealed that the engineered 14-hydroxycodeine strain produced thebaine, codeine, and other benzylioquinoline alkaloids from glucose and simple nitrogen sources present in the growth media (FIGS. 47A-47D).

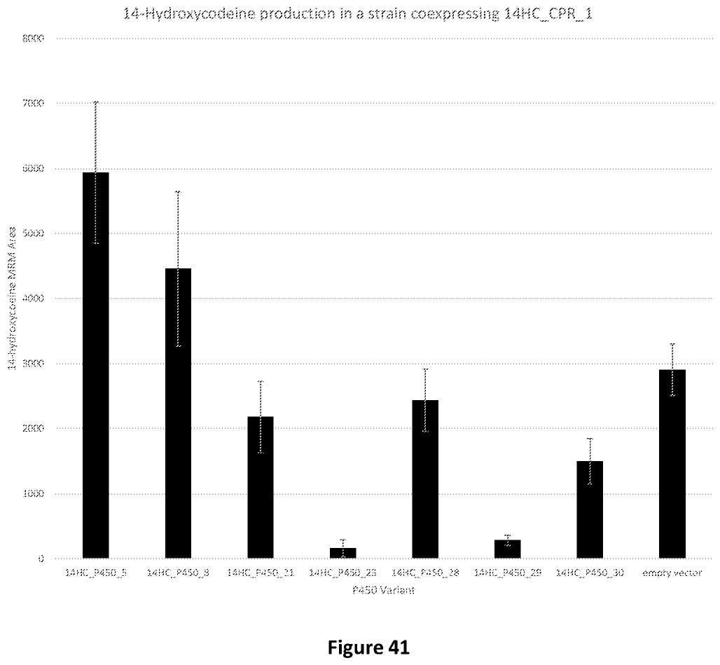

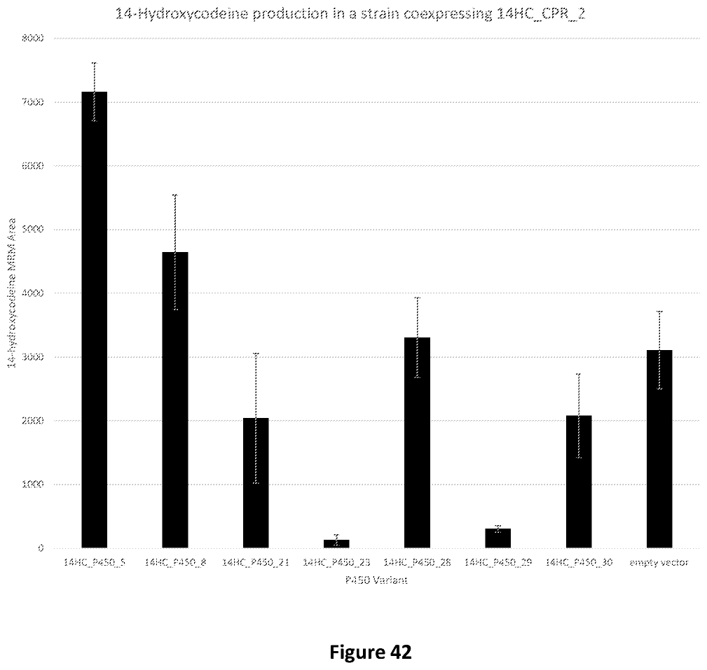

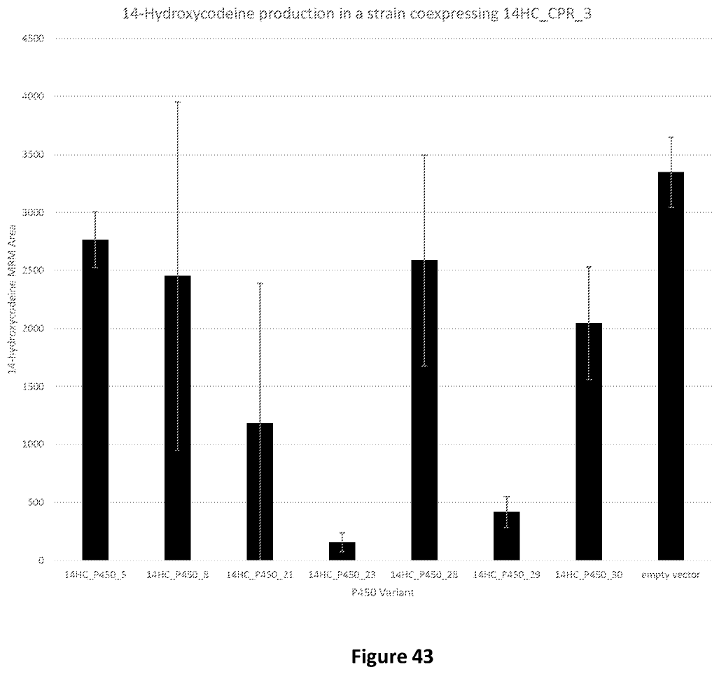

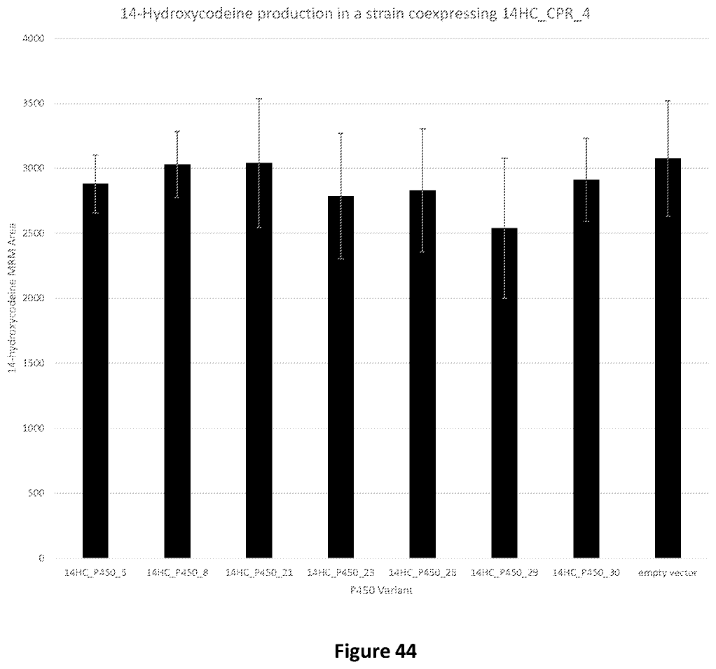

Seven C-14 hydroxylase candidates (14HC_P450_5, 14HC_P450_8, 14HC_P450_21, 14HC_P450_23, 14HC_P450_28, 14HC_P450_29, and 14HC_P450_30) were then selected for further examination with additional CPR candidates (14HC_CPR_1-5). Plasmids encoding a C-14 hydroxylase candidate and a CPR candidate were transformed into yeast strain YA3509, cultured, and selected as described above. Colonies were grown for an additional 72 hours at 28° C. as described above in replicates of 12. Next, 14-hydroxycodeine in the supernatant was measured by LC-MS/MS analysis as described above (FIGS. 40-44). From these cultures, candidate P450 14HC_P450_5 demonstrated significant production of 14-hydroxycodeine above the background level when co-expressed with one of two candidate CPRs (14HC_CPR_1 or 14HC_CPR_2) (FIGS. 41, 42). Empty vector controls (strain transformed with a plasmid as described above but lacking P450) showed that some 14-hydroxycodeine is spontaneously produced even in the absence of an additional P450 added for this purpose. Even accounting for the possibility of P450s present in the strain (both native yeast P450s and P450s added for the production of thebaine), 14-hydroxycodeine is produced above background level by 14HC_P450_5 under identical reaction conditions, in strains that differ in only this one enzyme. Furthermore, this activity is dependent on the presence of a co-expressed CPR partner. Because no 14-hydroxycodeine was produced above background when 14HC_P450_5 was co-expressed with 14HC_CPR 3, results shown in FIG. 43 indicate that an appropriate CPR partner must also be present (e.g., a fungal P450 paired with a fungal CPR).

The results presented in this Example demonstrate that we have identified an enzyme that performs a 14-hydroxylation enzymatically. Because both codeine and codeinone are present in the yeast strain, they are both potential substrates for this reaction. For example, codeinone could be converted to 14-hydroxycodeinone, which could then be reduced to 14-hydroxycodeine by a codeine reductase (COR), which is present in these strains. Additional substrates for this enzyme may include, but are not limited to, hydrocodone, norhydrocodone, norcodeine, norcodeinone, hydromorphone, morphinone, normorphinone, norhydromorphinone. Through one or more of these intermediates, in coordination with additional enzymes, fully de novo biosynthesis of noroxymorphone may be engineered thereby providing an improved route for the production of this essential pharmaceutical intermediate previously accessible only through laborious synthetic chemistry.

### Example 20: 14-Hydroxycodeine Produced by the Expression of Different CPRs

In order to function, P450 enzymes, including those described herein require an additional partner enzyme, a cytochrome P450 reductase (CPR). As shown above, it is reported that 14HC_P450_5 is active with two of the four CPRs tested: 14HC_CPR_1 and 14HC_CPR_2. Both of these CPRs are of fungal origin, whereas the other two CPRs tested, with both of which 14HC_P450_5 failed to demonstrate any activity, are of non-fungal origin. Furthermore, the level of activity seen with 14HC_CPR_1 and 14HC_CPR_2 differed significantly, showing the ability of the CPR to modulate the degree of activity seen as well.

Additional CPRs of different origins were then tested and are also shown in Table 18. The 14-hydroxylase enzyme 14HC_P450_41 was integrated into the ARG2 locus under the control of the TDH3 promoter to generate strain YA4997. Into this strain were transformed plasmids expressing different candidate CPR enzymes under the control of the pTDH3 promoter. Eight individual colonies were picked for each CPR candidate and used to inoculate 400 μL of uracil dropout synthetic complete media. These precultures were grown for 48 hours at 28° C. and 300 rpm. After 48 hours, the precultures had reached saturation, and 20 μL of each preculture were used to inoculate 380 μL of maltodextrin media. The production media in the present Example was prepared containing 3.13 g/L ammonium sulfate, 5 g/L monosodium glutamate, 2.75 g/L methionine, 0.03 g/L inositol, 0.1 g/L ampicillin, 19.2 g/L citric acid, 0.014 g/L EDTA, 0.55 mg/L copper(II)sulfate, 0.25 mg/L iron(III) chloride, 3.0 mg/L iron(III)sulfate heptahydrate, 3.0 mg/L manganese(II)sulfate monohydrate, 0.75 mg/L sodium molybdate(VI) dihydrate, 1.4 mg/L zinc sulfate heptahydrate, 3.0 mg/L boric acid, 0.6 mg/L potassium iodide, 0.25 g/L magnesium sulfate heptahydrate, 0.63 g/L potassium phosphate, 0.63 mg/L biotin, 1.5 mg/L p-aminobenzoic acid, 3.0 mg/L nicotinic acid, 8.1 mg/L pyridoxine hydrochloride, 3.0 mg/L thiamine hydrochloride, 13 mg/L D-calcium pantothenate, 14 mg/L adenine sulfate, 80 g/L maltodextrin and 2 units/L amylase. These production cultures were grown for 72 hours at 28° C. and 300 rpm. After 72 hours the cultures were clarified by centrifugation and the supernatants were analyzed by LCMS to determine the level of 14-hydroxycodeine produced.

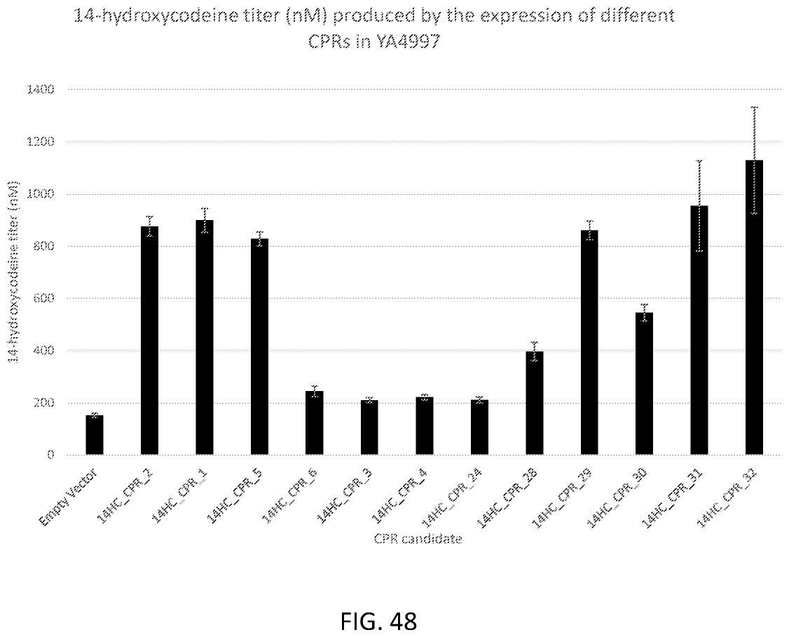

As shown in FIG. 48, 14hydroxycodeine production that was significantly above the background level was observed with 12 of the CPRs examined. Most of these active CPRs were of fungal origin, but two (14HC_CPR_3 and 14HC_CPR_24) were of animal origin, while one (14HC_CPR_4) was of plant origin. The fungal CPRs that had activity represented a diverse range of sequences, with 14HC_CPR_30 having less than 60% identity to 14HC_CPR 2, our originally reported most active CPR.

### Example 21: Mutagenesis Library of 14-Hydroxylase Variants

A mutagenesis library of 14-hydroxylase variants derived from 14HC_P450_5 was generated from error-prone PCR using the GeneMorph II Random Mutagenesis Kit (Agilent, 200550). The initial template plasmid for error-prone PCR was constructed by cloning 14HC_P450_5 into pUC19. Various PCR conditions were tested to optimize for a mutation rate of two to four mutations per amplicon. Based on these results, two condition combinations (1000 ng/20 cycles and 1000 ng/30 cycles) were selected to generate the final mutagenesis libraries. Each error-prone PCR reaction was prepared with 1× Mutazyme II reaction buffer, 800 uM dNTP mix, 0.2 uM forward primer, 0.2 uM reverse primer, 1000 ng template plasmid, 0.05 U/uL Mutazyme II DNA polymerase, and nuclease-free water to the final reaction volume. The error-prone PCR was run using the following thermocyler program: lx: 95° C. for 2 min; 20× or 30×: 95° C. for 30 sec →56° C. for 30 sec →72° C. for 1 min 30 sec; 1×: 72° C. for 10 min. Upon completion, 1 uL of Dpn1 (Thermo Scientific FD1703) was added to every 50 uL error-prone PCR product and incubated at 37 C for 3-4 hours. The digested amplicons were visualized on a 1% agarose gel, cut-out, and purified using the Zymoclean Gel DNA Recovery Kit (Zymo Research 4008). The resulting mutagenesis libraries were then transformed into yeast expressed under the pA36 plasmid and screened for 14-hydroxylase activity as previously described.

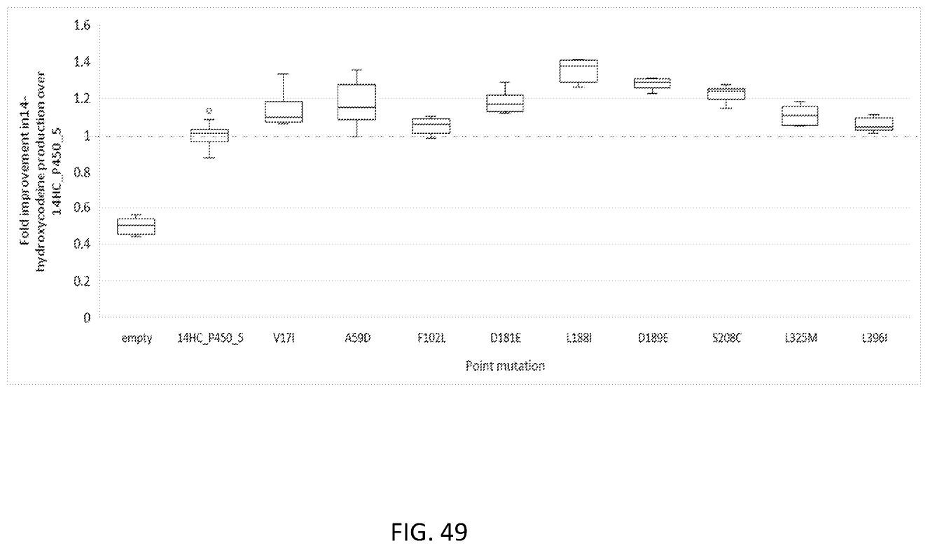

Fold improvement over 14HC_P450_5 in in vivo 14-hydroxycodeine production of individual point mutations (SEQ ID NOs: 180, 182, 196, 198, 200, 202, 204, 206, and 208) is shown in FIG. 49. Plasmids containing 14HC_P450_5 with individual mutations were transformed into YA4228 and assayed for 14-hydroxycodeine production in eight replicates. Outliers (defined as data points ±1.5*IQR) were excluded.

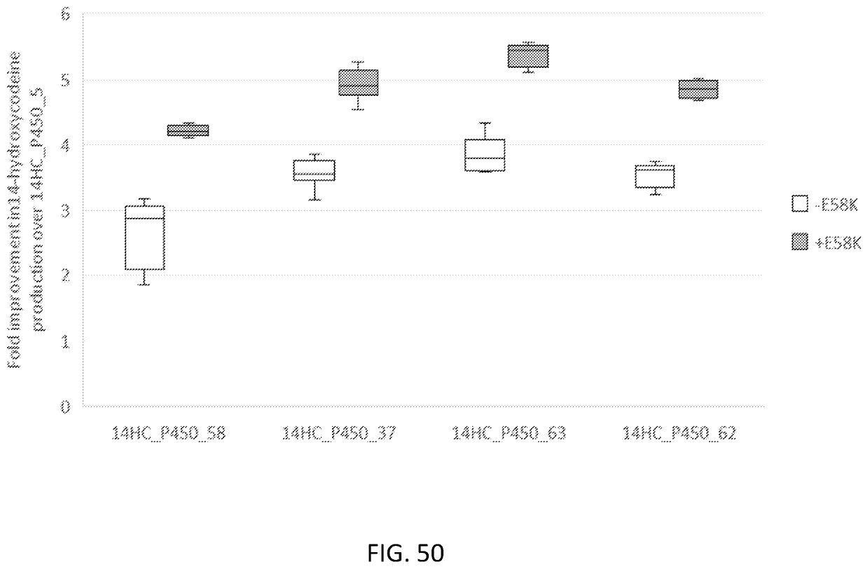

Comparisons of in vivo 14-hydroxycodeine titer improvements relative to 14HC_P450_5 upon addition of E58K point mutations were then performed and results are shown in FIG. 50. Plasmids containing engineered 14HC_P450_5 variants (14HC_P450_58 (A59D, D181E, L188I, D189E, S208C), 14HC_P450_37 (A59D, F102L, D181E, L188I, D189E, S208C, L325M), 14HC_P450_63 (A59D, F102L, D181E, L188I, D189E), 14_HC_P450_62 (V171, A59D, F102L, D181E, L188I, D189E, S208C); SEQ ID NOs: 226, 188, 236, and 234 were transformed into YA4228 (an engineered Saccharomyces cerevisiae strain producing codeine that was further engineered to be auxotrophic for tryptophan and express 14HC_CPR_2) and assayed for 14-hydroxycodeine production in eight replicates. Outliers (defined as data points ±1.5*IQR) were excluded.

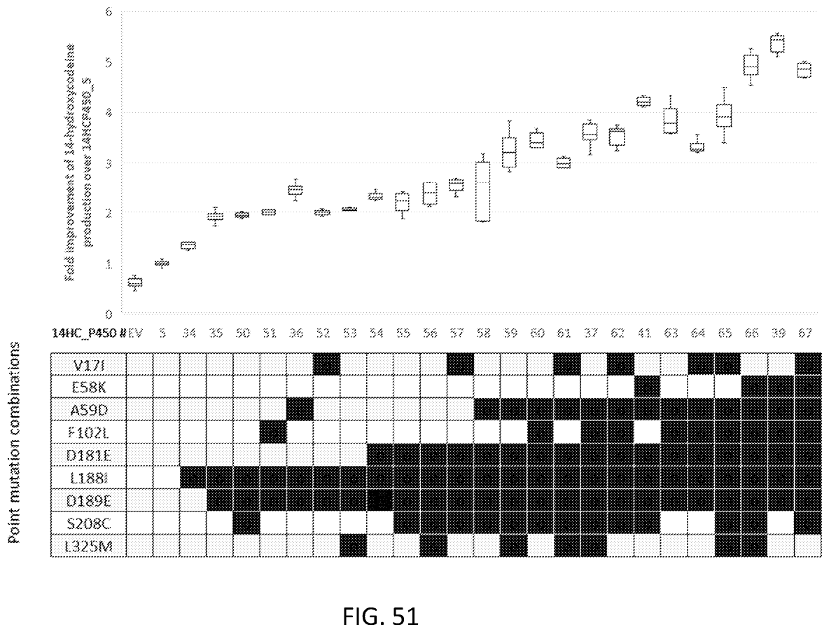

Various combinations of mutations were then examined. In vivo fold improvements over 14HC_P450_5 in 14-hydroxycodeine production of various combinations of 14HC_P450_5 point mutations are shown in FIG. 51. Plasmids containing 14HC_P450_5 variants were transformed into YA4228 as done in previous Examples and assayed for 14-hydroxycodeine production in eight replicates. EV indicates empty vector. The point mutations included are indicated with a blacked-out box for each variant. Outliers (defined as data points ±1.5*IQR) were excluded. The best performing combination, 14HC_P450_39 (E58K, A59D, F102L, D181E, L188I, and D189E), was improved 5.37-fold in comparison to 14HC_P450_5. Synergistic effects could be observed for certain combinations. 14HC_P450_35 (L188I, D189E) had 1.92-fold improvement compared to 14HC_P450_5 even though individually, L188I and D189E were 1.36 and 1.28-fold improved compared to 14HC_P450_5, respectively. 14HC_P450_36 (A59D, L188I, D189E) had 1.27-fold improvement compared to 14HC_P450_35 even though 14HC_P450_33 (A59D) was 1.16-fold improved compared to 14HC_P450_5.

### Example 22: In Vivo Oxycodone Production

To examine oxycodone production by strains expressing different engineered 14HC_P450_5 variants, plasmids encoding a engineered 14HC_P450_5 variant (14HC_P450_33-68) were transformed into yeast strain YA4229 (identical to YA4228 but the COR has been replaced by a copy of MorB to produce hydrocodone rather than codeine) using a commercially available transformation kit and were plated on selective solid media. A person of ordinary skill in the art will understand that other suitable yeast transformation protocols may be used. After 3 days of culturing at 30° C., colonies randomly selected and cultured and analyzed as described in Example 19.

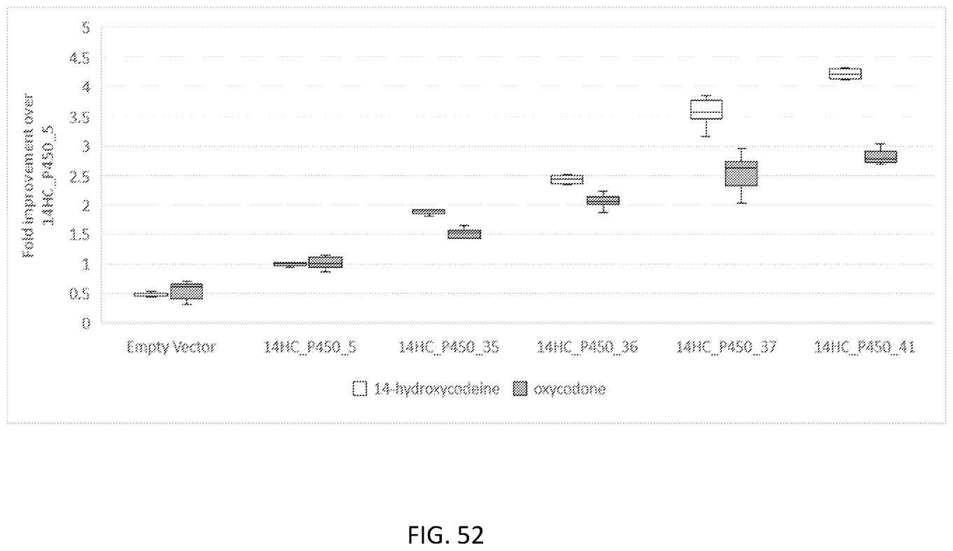

FIG. 52 shows In vivo fold improvements over 14HC_P450_5 in 14-hydroxycodeine production (white) and oxycodone production (gray) of engineered 14HC_P450_5 variants. Plasmids containing 14HC_P450_5 variants were transformed into YA4228 and YA4229 and assayed for 14-hydroxycodeine and oxycodone production, respectively, in eight replicates. Outliers (defined as data points ±1.5*IQR) were excluded.

### Example 23: Additional Data for 14-Hydroxylase Mutagenesis

Site-directed mutagenesis libraries of 14-hydroxylase variants were generated using 14HC_P450_36 (A59D, L188I, D189E) as the template. Libraries for each position were constructed by substituting the codon at the position with an NNK degenerate codon. Two rounds of PCR were used to construct each library.

In the first PCR, two gene fragments were amplified from 14HC_P450_36: one fragment comprised of bases from the 5′ end of 14HC_P450_36 to the position of interest, and one fragment comprised of bases from the position of interest to the 3′ end of the gene. NNK primers were designed so that each fragment overlapped 20-30 base pairs at the position of interest (Tm of 68-70C, as calculated by New England Bioscience's Tm calculator (version 1.16.5)). Each PCR reaction was prepared with 1×Q5 reaction buffer, 200 uM dNTP mix, 0.5 uM forward primer, 0.5 uM reverse primer, 0.02 ng/uL template plasmid, 0.02 U/uL Q5 High-Fidelity DNA Polymerase, and nuclease-free water to the final reaction volume. The PCR was run using the following thermocyler program: 1×: 98° C. for 2 min; 30×: 98° C. for 15 sec →67° C. for 45 sec →72° C. for 2 min; lx: 72° C. for 5 min. Upon completion, each 50 uL PCR reaction was treated with 1 uL of Dpn1 (Thermo Scientific FD1703) for 3-4 hours at 37 C. The digested amplicons were then pooled, purified (Zymo Research 4004) and then eluted in nuclease-free water in preparation for the second PCR.

The second PCR was prepared with 1×Q5 reaction buffer, 200 uM dNTP mix, 0.5 uM forward primer binding to the 5′ end of 14HC_P450_36, 0.5 uM reverse primer binding to the 3′ end of 14HC_P450_36, 9 uL purified amplicons, 0.02 U/uL Q5 High-Fidelity DNA Polymerase, and nuclease-free water to 100 μL. The second PCR was run using the following thermocyler program: lx: 98° C. for 2 min; 25×: 98° C. for 15 sec →67° C. for 45 sec →72° C. for 2 min; lx: 72° C. for 10 min. Amplicons from the second PCR were visualized on a 1% agarose gel, cut-out, and purified using the Zymoclean Gel DNA Recovery Kit (Zymo Research 4008). The resulting mutagenesis libraries were then transformed into yeast expressed under the pA36 plasmid and screened for 14-hydroxylase activity as previously described.

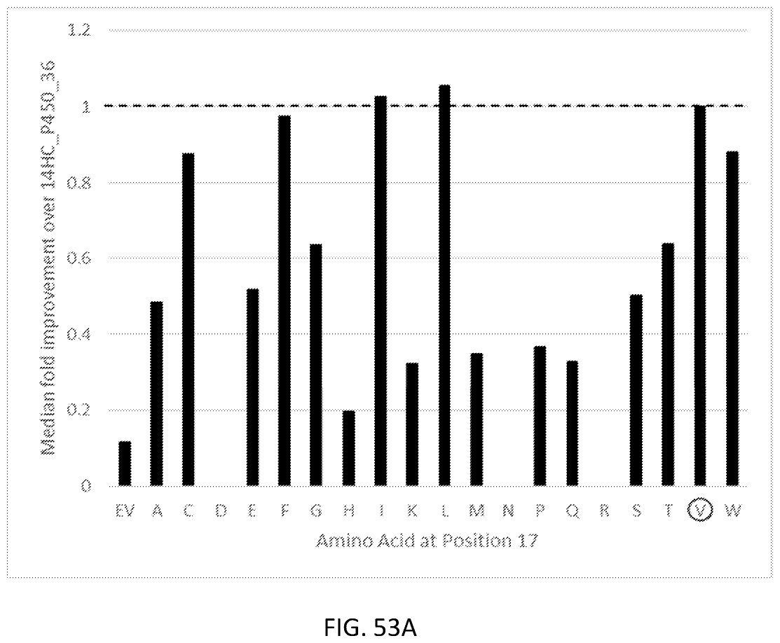

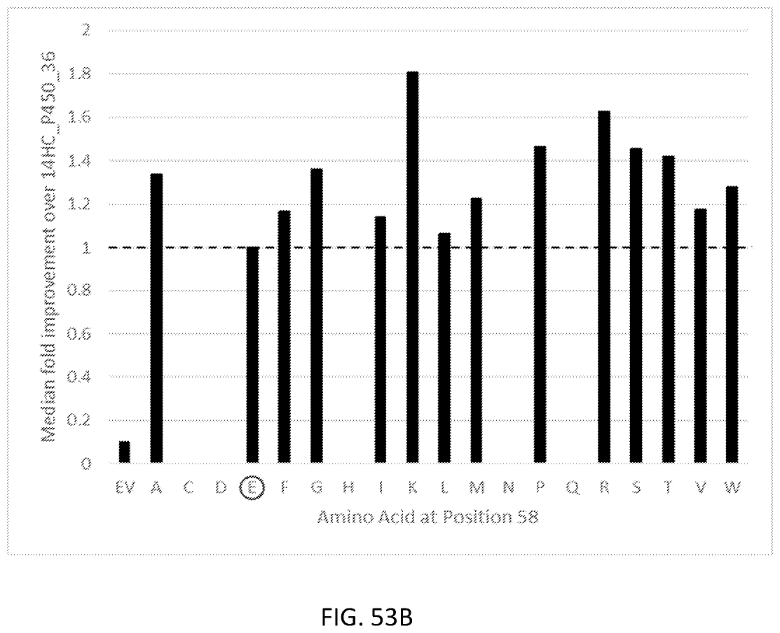

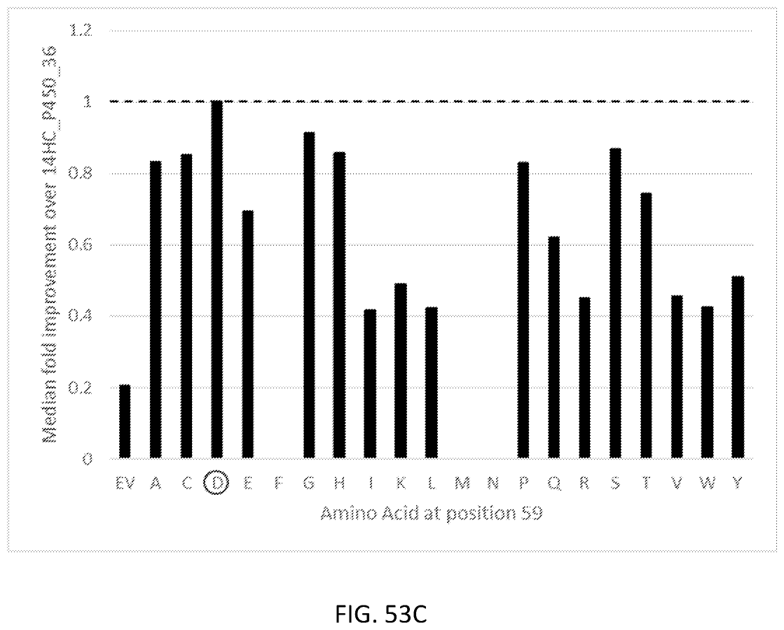

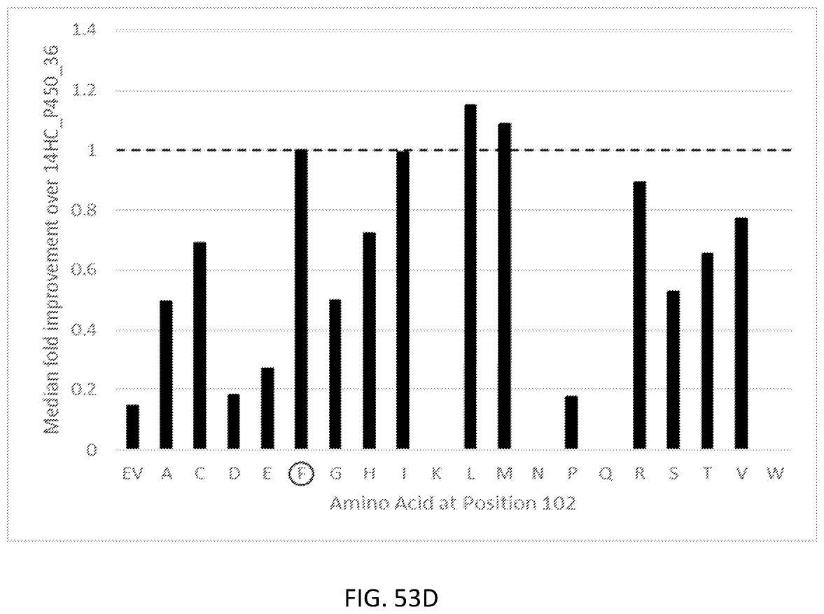

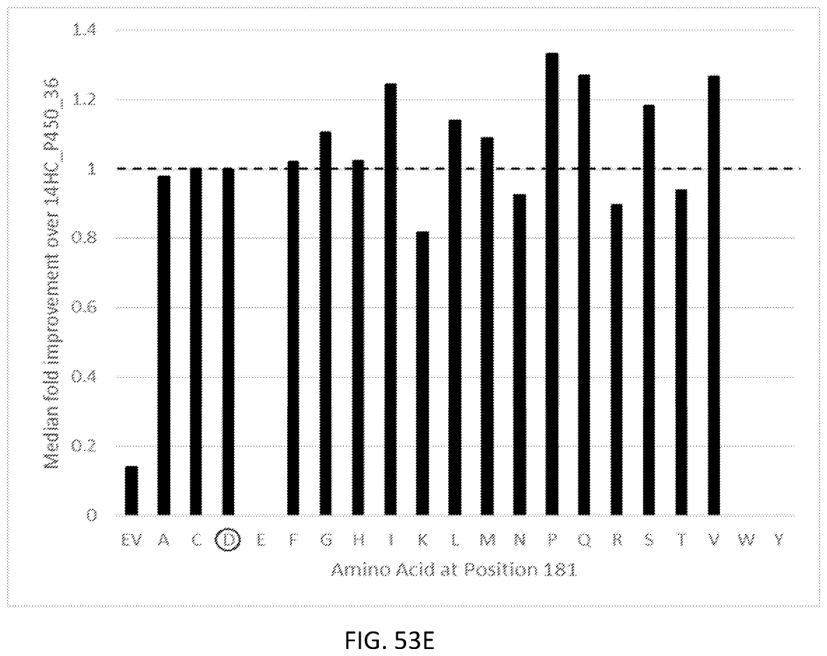

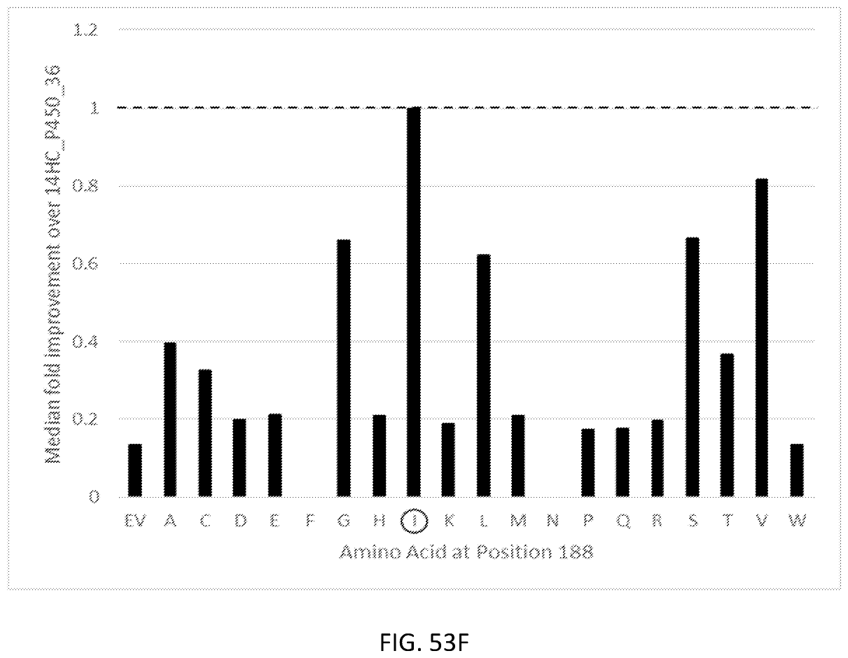

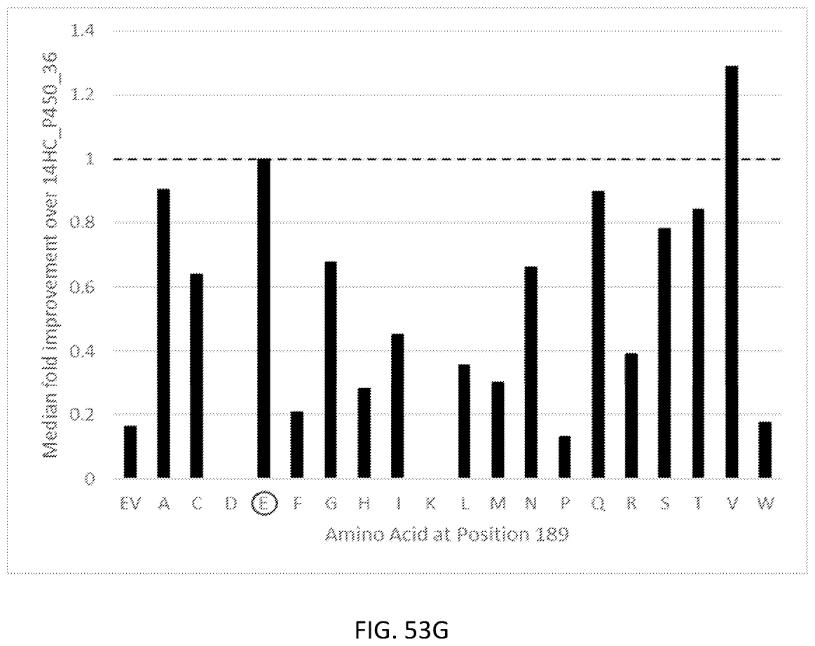

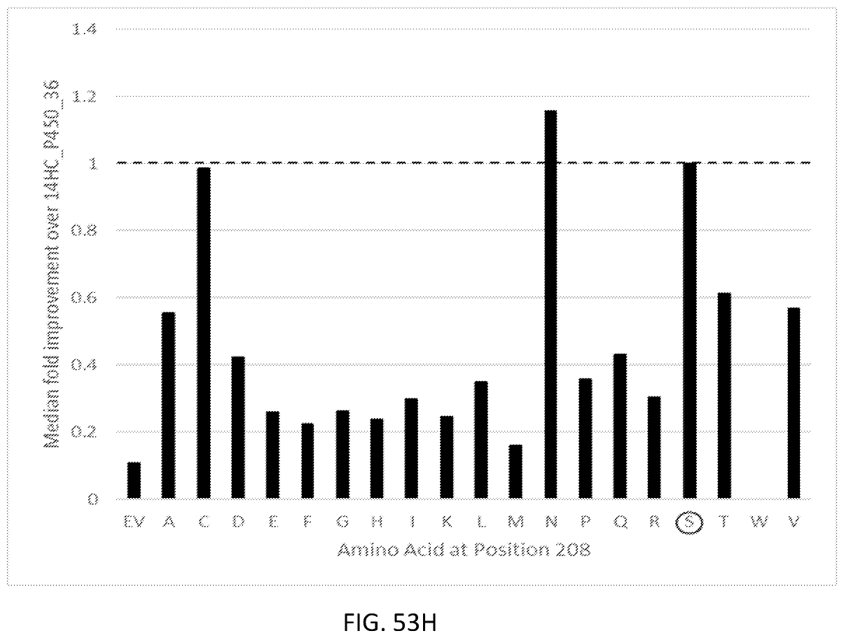

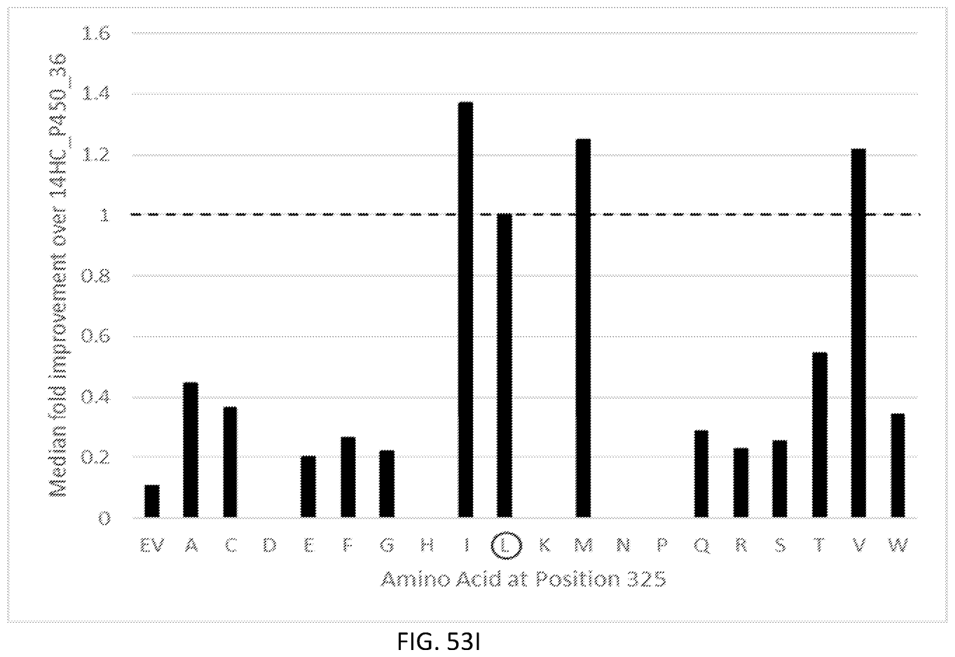

14-hydroxycodeine titers for each mutation were normalized for each amino acid position by dividing by the median of the parental control (14HC_P450_36 (A59D, L188I, D189E)) (FIGS. 53A-53I). Each parental control had eight replicates. Each bar represents the median fold improvement of 14-hydroxycodeine for the amino acid at that position. Lack of a bar indicates that the amino acid at that position was not tested. The amino acid at the indicated position in the parental control is circled on the x-axis of each figure. EV indicates empty vector.

As shown in FIG. 53A, at position 17, I and L improve 14-hydroxycodeine production relative to 14HC_P450_36. At position 58, all amino acids tested improve 14-hydroxycodeine production relative to 14HC_P450_36 (FIG. 53B). However, E58K shows the most improvement of the ones tested. At position 59, D is the best amino acid of the ones tested (FIG. 53C). At position 102, L and M improve 14-hydroxycodeine production relative to 14HC_P450_36. (FIG. 53D). At position 181, G, I, L, M, P, Q, S, and V are improved relative to 14HC_P450_36 (FIG. 53E). At position 188, I is the best amino acid of those tested (FIG. 53F). At position 189, V improves 14-hydroxycodeine production relative to 14HC_P450_36 (FIG. 53G). At position 208, N improves 14-hydroxycodeine production relative to 14HC_P450_36 (FIG. 53H). At position 325, I, M, and V improve 14-hydroxycodeine production relative to 14HC_P450_36 (FIG. 53I).

### Example 24: In Vitro Activity of 14-Hydroxylase Candidates on a Suite of BIAs

Yeast microsomes were prepared using a modified protocol from Pompon et al. (Pompon, Denis, et al. “[6] Yeast expression of animal and plant P450s in optimized redox environments.” Methods in enzymology. Vol. 272. Academic Press, 1996. 51-64.) Yeast strains containing 14HC-CPR2 integrated with either pA36-14HC_P450_5 or pA36 were grown on a YPD agar plate for 2 days. Single colonies were inoculated into 5 mL of YPD and grown at 30° C. at 200 rpm for 24h. 500 μL of this starter culture was added to 50 mL of YPGE (YPD, 2% v/v ethanol) and grown for 48h until OD 30-40. Cultures were then centrifuged for 4 minutes at 4000×g. Supernatant was removed and cell pellets were resuspended in 5 mL of TEK buffer (50 mM Tris-HCL, 1 mM EDTA, 0.1M KCl, pH 7.4) to rest at room temperature for 5 minutes. Cells were then centrifuged for 4 minutes at 4000×g and diluted in 6 mL of cold TES B buffer (50 mM Tris-HCL, 1 mM EDTA, 0.6M sorbitol, pH 7.4). Cells were lysed by hand shaking with 0.5 mm glass beads for 15 minutes. Lysed cells were removed and beads were rinsed with an additional 5 mL of cold TES B. The rinse was combined with the original lysed cell suspension and centrifuged at 5 C for 10 min at >10,000×g. The pellet was discarded and the supernatant was diluted with TES B to a final volume of 11.5 mL. 450 μL of 5M NaCl and 3 mL of 50% PEG were added to the supernatant. After gentle mixing, the solution was precipitated on ice for 30 minutes. The solution was centrifuged for 10 min at >10,000×g and the supernatant was discarded. The cell pellet was resuspended in 500 μL of cold TEG (50 mM Tris-HCL, 1 mM EDTA, 20% v/v glycerol, pH 7.4) and the Abs280 protein concentration was measured via Nanodrop. Microsomes were stored at −80° C. until used.

14HC_P450_5 was tested in vitro for its activity on codeinone, codeine, hydrocodone and hydromorphone. Reactions were run at room temperature in 50 mM sodium phosphate buffer (pH 7.5), 5 mM NADPH, 1 mM substrate and 5 mg/mL of microsomes from strains expressing either pA36-14HC005 or pA36 in 225 μL total reaction volume. The reaction was initiated with addition of enzyme and samples were taken at 30 seconds, 1.5 hours, 3 hours, and 24 hours. 50 μL samples were taken at each timepoint and immediately diluted 1:1 with methanol and stored at −20° C. until analysis. Samples were then diluted another 10 fold with 0.1% formic acid in water and analyzed with LC-MS as described previously.

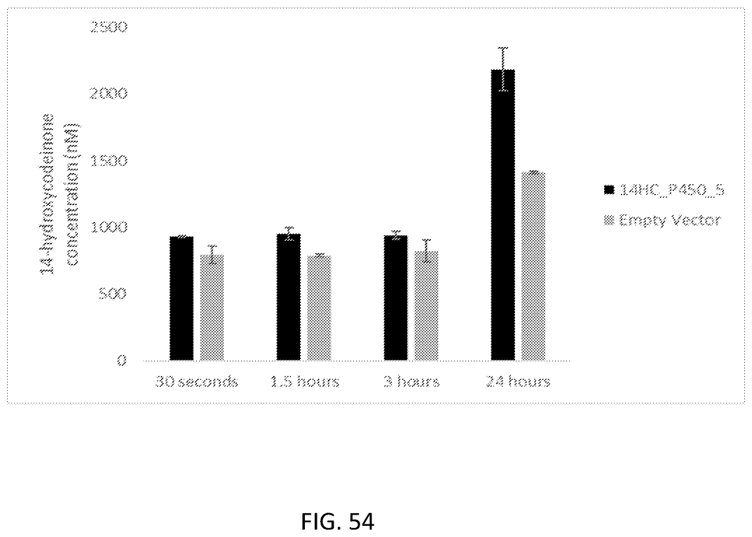

FIGS. 54-57 demonstrate 14HC_P450_5 can hydroxylate a variety of substrates such as codeinone, codeine, and hydrocodone. Error bars represent standard deviation of at least two replicates. These data demonstrate that 14HC_P450_5 is promiscuous and can be used to produce 14-hydroxycodeine and oxycodone both directly, via oxidation of codeine or hydrocodone, and indirectly via production of 14-hydroxycodeinone which can be further reduced to 14-hydroxycodeine or oxycodone.

FIG. 54 demonstrates in vitro production of 14-hydroxycodeinone when codeinone is used as substrate. There is a high level of spontaneous conversion of codeinone to 14-hydroxycodeinone when microsomes from strains expressing 14HC_P450_5 or empty vector used. However, there is significant in vitro production of 14-hydroxycodeinone by 14HC_P450_5 compared to the negative control, particularly at 24 hours.

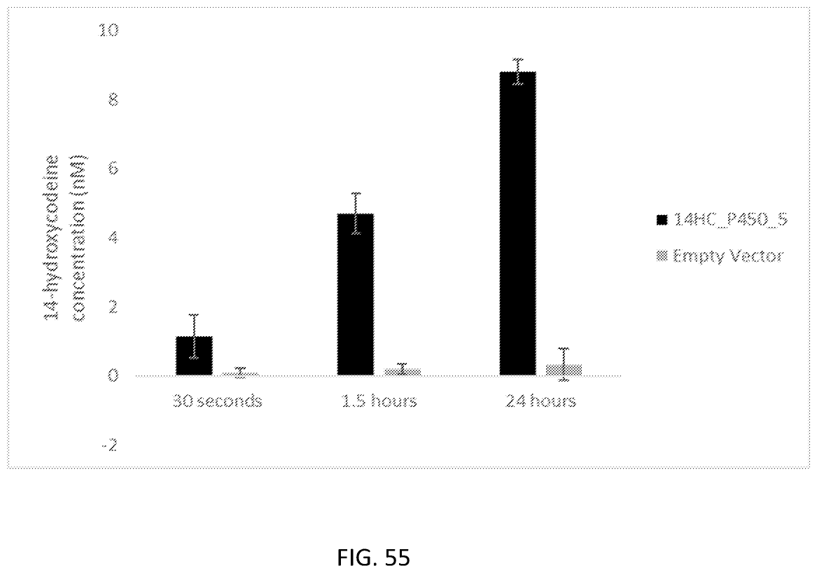

FIG. 55 demonstrates in vitro production of 14-hydroxycodeine when codeine is used as substrate. There is significant production of 14-hydroxycodeine by 14HC_P450_5 over time compared to the Empty Vector negative control. Note that for this assay, samples were not taken at 3 hours.

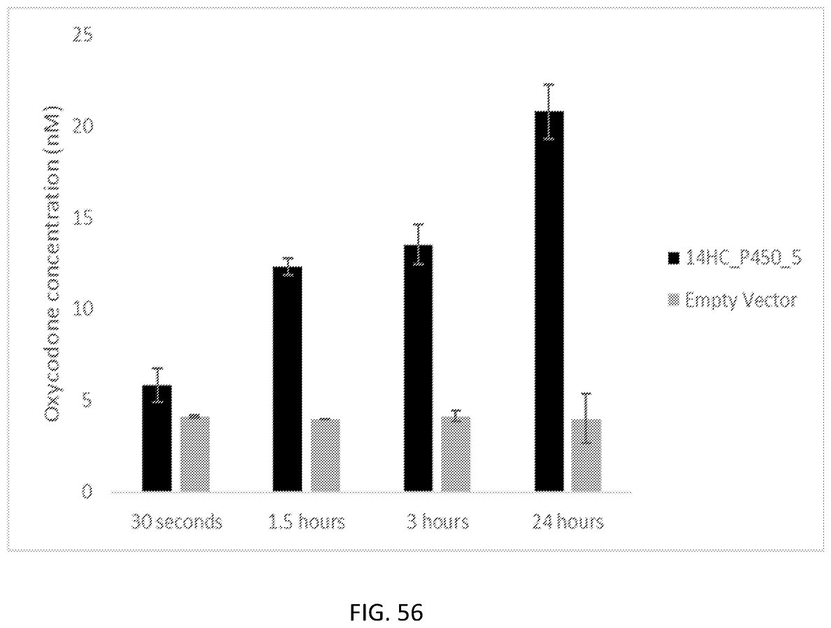

FIG. 56 demonstrates in vitro production of oxycodone when hydrocodone is used as substrate. There is significant production of oxycodone by 14HC_P450_5 over time compared to the Empty Vector negative control. There is a background of about 4 nM oxycodone which does not change over the course of the assay.

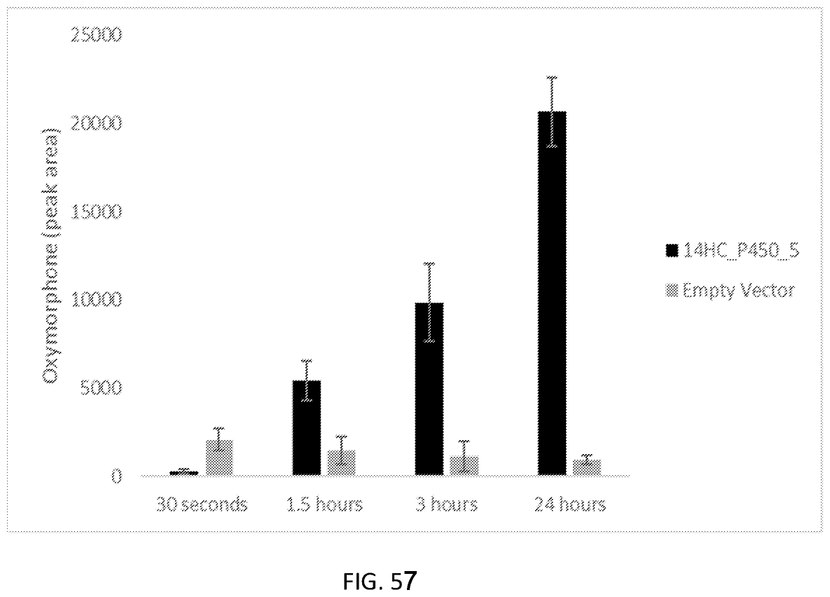

FIG. 57 demonstrates in vitro production of oxymorphone when hydromorphone is used as substrate. There is significant production of oxymorphone by 14HC_P450_5 overtime compared to the Empty Vector negative control. Oxymorphone levels are below the limit of quantification, but peak areas and transitions match the oxymorphone standard. This experiment demonstrates that 14HC_P450_5 is active on at least one 3-hydroxy version of an opioid.

TABLE 11
List of enzymes

| Enzyme | Abbrev | Catalyzed Reactions | Source organisms | Genbank # |
|---|---|---|---|---|
| Transketolase | TKL1 | fructose-6-phosphate + | Saccharomyces | NP_015399.1 |
| | | glyceraldehyde-3-phosphate | cerevisiae | |
| | | ↔ xylulose-5-phosphate + | | |
| | | erythrose-4-phosphate (EC | | |
| | | 2.2.1.1) | | |
| Glucose-6-phosphate | ZWF1 | glucose-6-phosphate → 6- | Saccharomyces | CAA96146.1 |
| dehydrogenase | | phosphogluconolactone (EC | cerevisiae | |
| | | 1.1.1.49) | | |
| Prephenate dehydrogenase | TYR1 | prephenate + NADP⁺ → 4- | Saccharomyces | CAA85127.1 |
| | | hydroxyphenylpyruvate + CO₂ + | cerevisiae | |
| | | NADPH (EC 1.3.1.13) | | |
| 3-deoxy-d-arabinose- | ARO4, | erythrose-4-phosphate + | Saccharomyces | CAA85212.1 |
| heptulosonate-7-phosphate | DAHP | PEP → DAHP (EC 2.5.1.54) | cerevisiae | |
| synthase | synthase | | | |
| Chorismate mutase | ARO7 | chorismate → prephenate | Saccharomyces | NP_015385.1 |
| | | (EC 5.4.99.5) | cerevisiae | |
| Phenylpyruvate decarboxylase | ARO10 | hydroxyphenylpyruvate → | Saccharomyces | NP_010668.3 |
| | | 4-HPAA (EC 4.1.1.80) | cerevisiae | |
| Alcohol dehydrogenase | ADH2-7, | 4-HPAA → tyrosol (EC | Saccharomyces | NP_014032.1, |
| | SFA1 | 1.1.1.90) | cerevisiae | AAT93007.1, |
| | | | | NP_011258.2, |
| | | | | NP_009703.3, |
| | | | | NP_014051.3, |
| | | | | NP_010030.1, |
| | | | | NP_010113.1 |
| Aldehyde oxidase | ALD2-6 | 4-HPAA → | Saccharomyces | NP_013893.1, |
| | | hydroxyphenylacetic acid | cerevisiae | NP_013892.1, |
| | | (EC 1.2.1.39) | | NP_015019.1, |
| | | | | NP_010996.2, |
| | | | | NP_015264.1 |
| Aryl-alcohol | AAD4, 6, | aromatic aldehyde + NAD⁺ | Saccharomyces | GAX67600, |
| dehydrogenase | 10, 14-16 | → aromatic alcohol + NADH | cerevisiae | GAX72034, |
| | | (EC 1.1.1.90) | | AAT93180, |
| | | | | AAS56234, |
| | | | | NP_012689, |
| | | | | GAX69843, |
| | | | | NP_014477, |
| | | | | AAS56127 |
| Aldehyde reductase | ARI1 | aldehyde → alcohol | Saccharomyces | KZV11071.1 |
| | | | cerevisiae | |
| Transcription regulator of | OPI1 | knockout phenotype: | Saccharomyces | KZV10697.1 |
| phospholipid biosynthetic | | overproduction of inositol | cerevisiae | |
| genes | | | | |
| Aromatic | ARO9 | hydroxyphenylpyruvate + L- | Saccharomyces | AEC14313.1 |
| aminotransferase | | alanine ↔ tyrosine + | cerevisiae | |
| | | pyruvate (EC 2.6.1.58) | | |
| Aromatic | ARO8 | hydroxyphenylpyruvate + | Saccharomyces | KZV11027.1 |
| aminotransferase | | glutamate ↔ tyrosine + | cerevisiae | |
| | | alpha-ketogluterate (EC | | |
| S-adenosylmethionine | SAM2 | ATP + L-methionine + H₂O | Saccharomyces | NP_010790.3 |
| synthetase | | ↔ phosphate + | cerevisiae | |
| | | diphosphate + S-adenosyl-L- | | |
| | | methionine (EC 2.5.1.6) | | |
| S-adenosyl-L- | SAH1 | S-adenosyl-L-homocysteine + | Saccharomyces | KZV11795.1 |
| homocysteine hydrolase | | H₂O ↔ L-homocysteine + | cerevisiae | |
| | | adenosine (EC 3.3.1.1) | | |
| Pyruvate | PDC1, | pyruvate → acetaldehyde + | Saccharomyces | EGA85775.1, |
| decarboxylase | PDC5, | CO₂ (EC 4.1.1.1) | cerevisiae | NP_013235.1, |
| | PDC6 | | | NP_011601.3 |
| Prephenate | PHA2 | prephenate → | Saccharomyces | QHB11078.1 |
| dehydratase | | phenylpyruvate + CO₂ + H₂O | cerevisiae | |
| | | (EC 4.2.1.51) | | |
| Phosphate | PTA | acetyl-CoA + phosphate ↔ | Clostridium kluyveri | WP_012101779.1 |
| acetyltransferase | | acetyl-phosphate + CoA (EC | | |
| | | 2.3.1.8) | | |
| Phosphoketolase | PK | fructose-6-phosphate + | Clostridium | WP_034583456.1, |
| | | phosphate → acetyl- | acetobutylicum, | WP_101428637.1, |
| | | phosphate + erythrose-4- | Bifidobacterium | WP_011680497.1 |
| | | phosphate + H₂O (EC | pseudolongum, | |
| | | 4.1.2.22) | Leuconostoc | |
| | | | mesenteroides | |
| 4-Hydroxyphenylacetaldehyde | HPAAS | tyrosine → 4-HPAA (EC | Petroselinum | Q06086.1, |
| synthase | | 4.1.1.108) | crispum, Rhodiola | MF674522.1 |
| | | | rosea | |
| Uridine 5′-diphospho- | UGT | UDP-glucose + a phenol → | Rhodiola rosea, Vitis | AUI41147.1, |
| glucosyltransferase | | UDP + an aryl beta-D- | vinifera, Oryza sativa | AUI41117.1, |
| | | glucoside (EC 2.4.1.35) | | XP_002285770.1, |
| | | | | XP015622802.1 |
| Steryl beta-glucosidase | EGH1 | Cholesteryl-beta-glucoside + | Saccharomyces | NP_012272.3 |
| | | H2O ↔ D-glucose + | cerevisiae | |
| | | colesterol (EC 3.2.1.104) | | |
| Arogenate | ADT | L-arogenate → L- | Papaver somniferum | XP_026414836.1 |
| dehydratase | | phenylalanine + CO₂ + H₂O | | |
| | | (EC 4.2.1.91) | | |
| Arogenate | AAT | L-arogenate + NAD⁺ → L- | Arabidopsis thaliana | NP_001331736.1 |
| dehydrogenase | | tyrosine + NADH + CO₂ + H⁺ | | |
| | | (EC 1.3.1.43) | | |
| Aspartate-prephenate | PAT | L-arogenate + oxaloacetate | Arabidopsis thaliana | NP_001031394.1 |
| aminotransferase | | ↔ prephenate + L- | | |
| | | aspartate (EC 2.6.1.78) | | |
| Tyrosinase | TYR | tyrosine → L-DOPA, L-DOPA | Ralstonia | NP_518458.1, |
| | | → dopaquinone (EC | solanacearum, | AJ223816, |
| | | 1.14.18.1) | Agaricus bisporus | |
| Tyrosine | TyrH | L-tyrosine → L-dopa (EC | Homo sapiens, Rattus | NP_000351.2, |
| hydroxylase | | 1.14.16.2) | norvegicus, Hypsibius | NP_036872.1, |
| | | | dujardini, Drosophila | OWA52330.1, |
| | | | melanogaster, | NP_476897.1, |
| | | | Schistosoma mansoni, | O17446.1, |
| | | | Schistosoma | ADV58258.1, |
| | | | japonicum, Canis | NP_001002966.1, |
| | | | lupus familiaris, Apis | NP_001011633.1 |
| | | | mellifera | |
| L-DOPA | DODC | L-DOPA → dopamine (EC | Pseudomonas putida, | AE015451.1, |
| decarboxylase | | 4.1.1.28) | Rattus norvegicus | NP_001257782.1 |
| Tyrosine/DOPA | TYDC | L-DOPA → dopamine (EC | Papaver somniferum | AAA97535 |
| decarboxylase | | 4.1.1.28) | | |
| | | tyrosine → tyramine (EC | | |
| | | 4.1.1.25) | | |
| Monoamine oxidase | MAO | dopamine → 3,4-DHPA (EC | E. coli, Homo sapiens, | J03792, D2367, |
| | | 1.4.3.4) | Micrococcus luteus | AB010716.1 |
| Norcoclaurine synthase | NCS | 4-HPAA + dopamine → S- | Coptis japonica, | BAF45337.1, |
| | | norcoclaurine (EC 4.2.1.78) | Papaver somniferum, | AB267399.2, ACI45396.1, |
| | | 3,4-DHPA + dopamine → S- | Papaver bracteatum, | ACO90258.1, |
| | | norlaudanosoline | Thalicitum flavum, | ACO90247.1, |
| | | | Corydalis saxicola | AEB71889.1 |
| Norcoclaurine 6-O- | 6OMT | Norcoclaurine → coclaurine | P. somniferum | AY268894 |
| methyltransferase | | Norlaudanosoline | T. flavum | AY610507 |
| | | → 3′hydroxycoclaurine | Coptis japonica* | D29811 |
| | | (EC 2.1.1.128) | | |
| Coclaurine-N- | CNMT | Coclaurine → N- | P. somniferum | AY217336 |
| methyltransferase | | methylcoclaurine | T. flavum | AY610508 |
| | | 3′hydroxycoclaurine | Coptis japonica* | AB061863 |
| | | → 3′-hydroxy-N- | | |
| | | methylcoclaurine | | |
| | | (EC 2.1.1.140) | | |
| 4′-O-methyltransferase | 4′OMT | 3′-hydroxy-N-methylcoclaurine | P. somniferum | AY217333, |
| | | → Reticuline ( EC 2.1.1.116) | T. flavum | AY217334 |
| | | | Coptis japonica* | AY610510 |
| | | | | D29812 |
| Cytochrome P450 80B1 | CYP80B1 | N-methylcoclaurine → 3′- | P. somniferum, | AAF61400.1 |
| | | hydroxy-N-methylcoclaurine | E. californica, | AAC39453.1 |
| | | (EC 1.14.13.71) | T. flavum, | AAU20767.1 |
| | | | P. bracteatum | ACO90224.1 |
| GTP cyclohydrolase | FOL2 | GTP → dihydroneopterin | Saccharomyces | CAA97297.1, |
| | | triphosphate (EC 3.5.4.16) | cerevisiae, Homo | NP_001019195.1, |
| | | | sapiens, | NP_032128.1 |
| | | | Mus musculus | |
| 6-pyruvoyl | PTPS | dihydroneopterin | Rattus norvegicus, | AAH59140.1, |
| tetrahydrobiopterin (PTP) | | triphosphate → PTP (EC | Homo sapiens, | BAA04224.1, |
| synthase | | 4.2.3.12) | Mus musculus | AAH29013.1 |
| Sepiapterin reductase | SepR | PTP → BH4 (EC 1.1.1.153) | Rattus norvegicus, | NP_062054.1, |
| | | | Homo sapiens, | NP_003115.1, |
| | | | Mus musculus | NP_035597.2 |
| 4a- | PCD | 4a- | Rattus norvegicus, | NP_001007602.1, |
| hydroxytetrahydrobiopterin | | hydroxytetrahydrobiopterin | Homo sapiens, | AAB25581.1, |
| (pterin-4α-carbinolamine) | | → H2O + quinoid | Mus musculus | NP_079549.1 |
| dehydratase | | dihydropteridine (EC | | |
| | | 4.2.1.96) | | |
| Quinoid dihydropteridine | QDHPR | quinoid dihydropteridine → | Rattus norvegicus, | AAH72536.1, |
| reductase | | BH4 (EC 1.5.1.34) | Homo sapiens, | NP_000311.2, |
| | | | Mus musculus | AAH02107.1 |
| Dihydrofolate reductase | DHFR | 7,8-Dihydrobiopterin → | Rattus norvegicus, | AF318150.1 |
| | | 5,6,7,8-Tetrahydrobiopterin | Homo sapiens | |
| | | (BH4) | | |
| | | (EC 1.5.1.3) | | |
| 1-benzylisoquinoline alkaloid | DRS-DRR | (S)-reticuline −> (R)-reticuline | Papaver | PODKI7.1, |
| epimerase (cytochrome P450 | (CYP- | (S)-1-benzylisoquinoline−>(R)-1- | bracteatum, | AKO60175.1, |
| 82Y2-codeinone reductase; | COR) | benzylisoquinoline | Papaver | AKO60180.1, |
| dehydroreticuline synthase- | | (EC 1.5.1.27) | somniferum, | AKO60179.1, |
| dehydroreticuline reductase) | | | Papaver setigerum, | AKO60175.1 |
| | | | Chelidonium majus | |
| (R)-reticuline, NADPH:oxygen | SalSyn | (R)-reticuline + NADPH + H+ + | Papaver | EF451150 |
| oxidoreductase (C-C phenol- | | O2 → salutaridine + NADP+ + 2 | somniferum, | (Ref PMID |
| coupling), also known as | | H2O | Papaver spp | 22424601) |
| salutaridine synthase | | (EC 1.14.21.4) | Chelidonium majus | |
| salutaridinol:NADP+ 7- | SalR | salutaridinol + NADP+ ↔ | Papaver | DQ316261, |
| oxidoreductase, also known | | salutaridine + NADPH + H+ | somniferum, | EF184229 |
| as salutaridine reductase | | (EC 1.1.1.248) | Papaver | (Ref PMID |
| | | | bracteatum, | 22424601) |
| | | | Papaver spp | |
| | | | Chelidonium majus | |
| acetyl-CoA:salutaridinol 7-O- | SalAT | acetyl-CoA + salutaridinol → | Papaver | AF339913, |
| acetyltransferase | | CoA + 7-O- acetylsalutaridinol | somniferum, | FJ200355, |
| | | (EC 2.3.1.150) | Papaver | FJ200358, |
| | | | bracteatum, | FJ200356, |
| | | | Papaver orientale, | JQ659008 |
| | | | Papaver spp | |
| Thebaine synthase | TS | 7-O-acetylsalutaridinol → | Papaver | AWQ63979, |
| | | thebaine + acetate | somniferum, | AWQ63980 |
| | | (EC 4.2.99.24) | Papaver | |
| | | | bracteatum, | |
| | | | Papaver orientale, | |
| | | | Papaver setigerum, | |
| | | | Papaver spp | |
| Thebaine 6-O demethylase | T6ODM | thebaine → neopinone (EC | Papaver | GQ500139.1 |
| | | 1.14.11.31) | somniferium, | |
| | | | Papaver spp. | |
| Neopinone Isomerase | NPI | neopinone → codeinone | Papaver | XP_026424272.1 |
| | | neomorphinone → | somniferum, | |
| | | morphinone | Papaver | |
| | | (EC 5.3.3.—) | bracteatum, | |
| | | | Papaver orientale, | |
| | | | Papaver setigerum, | |
| | | | Papaver rhoeas, | |
| | | | Papaver spp | |
| Codeinone reductase | COR | codeinone→ codeine (EC | Papaver | AF108432.1 |
| | | 1.1.1.247), | somniferium, | AF108433.1 |
| | | neopinone→ neopine | Papaver spp. | AF108434.1 |
| | | | | AF108435.1 |
| Codeine O-demethylase | CODM | codeine→ morphine (EC | Papaver | GQ500141.1 |
| | | 1.14.11.32), | somniferium, | |
| | | neopine→ neomorphine | Papaver spp. | |
| Morphine dehydrogenase | morA | morphine → morphinone (EC | Pseudomonas | M94775.1 |
| | | 1.1.1.218), | putida | |
| | | codeinone → codeine (EC | | |
| | | 1.1.1.247) | | |
| Morphinone reductase | morB | codeinone → hydrocodone | Pseudomonas | U37350.1 |
| | | morphinone → | putida | |
| | | hydromorphone (EC 1.3.1.—) | | |
| NADPH:hemoprotein | ATR1, | NADPH + H+ + n oxidized | Arabidopsis | NM118585, |
| oxidoreductase, also known | CPR | hemoprotein → NADP+ + | thaliana, E. | CAB58576.1, |
| as cytochrome P450 | | n reduced hemoprotein (EC | californica, P. | CAB58575.1, |
| reductase | | 1.6.2.4) | somniferum, H. | AAC05021.1, |
| | | | sapiens, S. | AAC05022.1 many |
| | | | cerevisiae, P. | others (Ref PMID |
| | | | bracteatum, | 19931102) |
| | | | Papaver spp., all | |
| | | | plants | |
| Cytochrome P450, family 2, | CYP2D6 | Promiscuous oxidase, can | Homo sapiens | BC067432 |
| subfamily D, polypeptide 6 | | perform | | |
| | | (R)-reticuline + NADPH + H+ + | | |
| | | O2 → salutaridine + NADP+ + 2 | | |
| | | H2O among other reactions | | |
| | | (EC 1.14.14.1) | | |
| S-adenosyl-L-methionine:(S)- | S9OMT | S-adenosyl-L-methionine + (S)- | Thalictrum flavum | AY610512, |
| scoulerine 9-O- | | scoulerine → S- adenosyl-L- | subsp. glaucum, | D29809, |
| methyltransferase | | homocysteine + (S)- | Papaver | EU980450, |
| | | tetrahydrocolumbamine | somniferum, Coptis | JN185323 |
| | | (EC 2.1.1.117) | japonica, Coptis | |
| | | | chinensis, | |
| | | | Thalictrum spp, | |
| | | | Coptis spp, | |
| | | | Papaver spp | |
| Tetrahydroprotoberberine- | TNMT | Stylopine → cis-N- | P. somniferum | DQ028579 |
| N-methyltransferase | | methylstylopine ( EC | E. californica | EU882977 |
| | | 2.1.1.122) | P. bracteatum | EU882994 |
| | | Canadine → N-methylcanadine | A. mexicana | HQ116698 |
| Cheilanthifoline synthase | CFS | Cheilanthifoline → | P. somniferum | GU325749 |
| | | stylopine | E. californica | AB434654 |
| | | (EC 1.14.21.1) | P. bracteatum | EF451152 |
| | | | A. mexicana | |
| Stylopine synthase | STS | Stylopine → cis-N- | P. somniferum | GU325750 |
| | | methylstylopine | E. californica | AB126257 |
| | | (EC 2.1.1.122) | P. bracteatum | EF451151 |
| | | | A. mexicana | |
| Cis-N-methylstylopine 14- | MSH | Cis-N-methylstylopine | P. somniferum | KC154003 |
| hydroxylase | | →protopine | E. californica | |
| | | (EC 1.14.13.37) | P. bracteatum | |
| | | | A. mexicana | |
| Protopine-6-hydroxylase | P6H | Protopine → 6- | E. californica | AB598834 |
| | | hydroxyprotopine | P. somniferum | AGC92397 |
| | | (EC 1.14.13.55) | P. bracteatum | |
| | | | A. mexicana | |
| Dihydrobenzophenanthridine | DBOX | Dihydrosanguinarine → | P. somniferum | AGL44336, |
| oxidase | | sanguinarine | E. californica | AGL44335, |
| | | (EC 1.5.3.12) | P. bracteatum | AGL44334 |
| | | | A. mexicana | |
| (S)- | STOX | (S)-tetrahydroberberine + 2 O₂ | Berberis wilsonae, | HQ116697, |
| tetrahydroprotoberberine | | → berberine + 2 H₂O₂ | Coptis japonica, | AB564543 |
| oxidase | | (EC 1.3.3.8) | Berberis spp, | |
| | | | Coptis spp | |
| (S)-tetrahydrocolumbamine, | CAS | (S)-tetrahydrocolumbamine + | Thalictrum flavum | AY610513, |
| NADPH:oxygen | | NADPH + H+ + O2 → (S)- | subsp. glaucum, | AB026122, |
| oxidoreductase | | canadine + NADP+ + 2 H2O | Coptis japonica, | AB374407, |
| (methylenedioxy-bridge- | | (EC 1.14.21.5) | Thalictrum spp, | AB374408 |
| forming), also known as (S)- | | | Coptis spp | |
| canadine synthase | | | | |
| (S)-reticuline:oxygen | BBE | (S)-reticuline + O2 → (S)- | Papaver somniferum, | AF025430, |
| oxidoreductase (methylene- | | scoulerine + H2O2 | Argemone mexicana, | EU881889, |
| bridge-forming), also known | | (EC 1.21.3.3) | Eschscholzia | EU881890, |
| as berberine bridge enzyme | | | californica, Berberis | S65550 |
| | | | stolonifera, | AF005655, |
| | | | Thalictrum flavum | AF049347, |
| | | | subsp. glaucum, | AY610511, |
| | | | Coptis japonica, | AB747097 |
| | | | Papaver spp, | |
| | | | Eschscholzia spp, | |
| | | | Berberis spp, | |
| | | | Thalictrum spp | |
| Berbamunine synthase | CYP80A1 | (S)-N-methylcoclaurine + (R)-N- | Berberis stolonifera, | AAC48987, |
| | | methylcoclaurine −> | Capsicum chinense, | PHU28278, |
| | | berbamunine | Quercus suber | POF05239 |
| | | (EC 1.14.21.3) | | |
| | | 1-benzylisoquinoline alkaloid + 1- | | |
| | | benzylisoquinoline alkaloid −> | | |
| | | bis-benzylisoquinoline alkaloid | | |
| Protopine O-dealkylase | PODA | O,O-demethylenation of | P. somniferum, | GQ500140.1 |
| | | canadine, stylopine and | Papaver spp. | |
| | | berberine | | |
| Reticuline N- | RNMT | reticuline→tembetarine | Papaver | KX369612.1 |
| methyltransferase | | | somniferum, | |
| | | | Papaver spp. | |
| Papaverine 7-O-demethylase | P7OMT | papaverine→pacodine | Papaver | KT159979.1 |
| | | | somniferum, | |
| | | | Papaver spp. | |
| 3-O-demethylase | 3ODM | oxycodone→oxymorphone | Papaver | |
| | | hydrocodone→hydromorphone | somniferum, | |
| | | dihydrocodeine→dihydromorphine | Papaver | |
| | | 14-hydroxycodeine→14- | bracteatum, | |
| | | hydroxymorphine | Papaver rhoeas, | |
| | | codeinone→morphinone | Papaver spp. | |
| | | 14-hydroxycodeinone→14- | | |
| | | hydroxymorphinone | | |
| N-demethylase (in some | NDM | Codeine→Norcodeine | Bacillus | |
| cases the NDM activity is | | Morphine→Normorphine | megaterium, Homo | |
| encoded in BM3 or an | | Oxycodone→Noroxycodone | sapiens, Papaver | |
| engineered variant of BM3) | | Oxymorphone→Noroxymorphone | somniferum, | |
| | | Thebaine→Northebaine | Papaver spp., | |
| | | Oripavine→Nororipavine | Chelidonium majus, | |
| | | Hydrocodone→Norhydrocodone | Stylophorum | |
| | | Hydromorphone→Norhydromorphone | diphyllum, Nigella | |
| | | Dihydrocodeine→Nordihydrocodeine | sativa, Hydrastis | |
| | | Dihydromorphine→Nordihydromorphine | canadensis, | |
| | | 14-hydroxycodeine→Nor-14- | Glaucium flavum, | |
| | | hydroxycodeine | Eschscholzia | |
| | | 14-hydroxymorphine→Nor-14- | californica, | |
| | | hydroxymorphine | Menispermum | |
| | | Codeinone→Norcodeinone | canadense, Papaver | |
| | | Morphinone→Normorphinone | bracteatum | |
| | | 14-hydroxycodeinone→Nor-14- | | |
| | | hydroxycodeinone | | |
| | | 14-hydroxymorphinone→Nor- | | |
| | | 14-hydroxymorphinone | | |
| N-methyltransferase | NMT | Norcodeine→codeine | Papaver spp., | |
| | | Normorphine→morphine | Chelidonium majus, | |
| | | Noroxycodone→oxycodone | Thalictrum flavum, | |
| | | Noroxymorphone→noroxymorphone | Coptis japonica, | |
| | | Northebaine→thebaine | Papaver | |
| | | Nororipavine→oripavine | somniferum, | |
| | | Norhydrocodone→hydrocodone | Eschscholzia | |
| | | Norhydromorphone→ | californica, Papaver | |
| | | Hydromorphone | bracteatum, | |
| | | Nordihydrocodeine→ | Argenome | |
| | | Dihydrocodeine | mexicana, Glaucium | |
| | | Nordihydromorphine→ | flavum, Sanguinaria | |
| | | Dihydromorphine | canadensis, | |
| | | Nor-14-hydroxycodeine→ 14- | Corydalis | |
| | | hydroxycodeine | chelanthifolia, | |
| | | Nor-14-hydroxymorphine→ 14- | Nigella sativa, | |
| | | hydroxymorphine | Jeffersonia diphylla, | |
| | | Norcodeineone→ Codeineone | Berberis thunbergii, | |
| | | Normorphinone→ Morphinone | Mahonia | |
| | | Nor-14-hydroxy-codeinone→ 14- | aquifolium, | |
| | | hydroxycodeinone | Menispermum | |
| | | (Nor-14-hydroxy-morphinone→ | canadense, | |
| | | 14-hydroxymorphinone | Tinospora cordifolia, | |
| | | | Cissampelos | |
| | | | mucronata, | |
| | | | Cocculus trilobus | |
| N-methyltransferase | NMT | Norcodeine→codeine | Papaver spp., | |
| | | Normorphine→morphine | Chelidonium majus, | |
| | | Noroxycodone→oxycodone | Thalictrum flavum, | |
| | | Noroxymorphone→noroxymorphone | Coptis japonica, | |
| | | Northebaine→thebaine | Papaver | |
| | | Nororipavine→oripavine | somniferum, | |
| | | Norhydrocodone→hydrocodone | Eschscholzia | |
| | | Norhydromorphone→ | californica, Papaver | |
| | | Hydromorphone | bracteatum, | |
| | | Nordihydrocodeine→ | Argenome | |
| | | Dihydrocodeine | mexicana, Glaucium | |
| | | Nordihydromorphine→ | flavum, Sanguinaria | |
| | | Dihydromorphine | canadensis, | |
| | | Nor-14-hydroxycodeine→ 14- | Corydalis | |
| | | hydroxycodeine | chelanthifolia, | |
| | | Nor-14-hydroxymorphine→ 14- | Nigella sativa, | |
| | | hydroxymorphine | Jeffersonia diphylla, | |
| | | Norcodeineone→ Codeineone | Berberis thunbergii, | |
| | | Normorphinone→ Morphinone | Mahonia | |
| | | Nor-14-hydroxy-codeinone→ 14- | aquifolium, | |
| | | hydroxycodeinone | Menispermum | |
| | | Nor-14-hydroxy-morphinone→ | canadense, | |
| | | 14-hydroxymorphinone | Tinospora cordifolia, | |
| | | | Cissampelos | |
| | | | mucronata, | |
| | | | Cocculus trilobus | |
| N-allyltransferase | NAT | Norcodeine→N-allyl-norcodeine | Papaver spp., | |
| | | Normorphine→N-allyl- | Chelidonium majus, | |
| | | normorphine | Thalictrum flavum, | |
| | | Noroxycodone→N-allyl- | Coptis japonica, | |
| | | noroxycodone | Papaver | |
| | | Noroxymorphone→N-allyl- | somniferum, | |
| | | nornoroxymorphone | Eschscholzia | |
| | | Northebaine→N-allyl- | californica, Papaver | |
| | | northebaine | bracteatum, | |
| | | Nororipavine→N-allyl- | Argenome | |
| | | nororipavine | mexicana, Glaucium | |
| | | Norhydrocodone→N-allyl- | flavum, Sanguinaria | |
| | | norhydrocodone | canadensis, | |
| | | Norhydromorphone→ N-allyl- | Corydalis | |
| | | norhydromorphone | chelanthifolia, | |
| | | Nordihydrocodeine→ N-allyl- | Nigella sativa, | |
| | | nordihydrocodeine | Jeffersonia diphylla, | |
| | | Nordihydromorphine→ N-allyl- | Berberis thunbergii, | |
| | | nordihydromorphine | Mahonia | |
| | | Nor-14-hydroxycodeine→ N- | aquifolium, | |
| | | allyl-nor-14-hydroxycodeine | Menispermum | |
| | | Nor-14-hydroxymorphine→ N- | canadense, | |
| | | allyl-nor-14-hydroxymorphine | Tinospora cordifolia, | |
| | | Norcodeineone→ N-allyl- | Cissampelos | |
| | | norcodeineone | mucronata, | |
| | | | Cocculus trilobus | |
| N- | CPMT | Norcodeine→N(cyclopropylmethyl)norcodeine | Papaver spp., | |
| cyclopropylmethyltransferase | | Normorphine→N(cyclopropylmethyl) | Chelidonium majus, | |
| | | normorphine | Thalictrum flavum, | |
| | | Noroxycodone→N(cyclopropylmethyl) | Coptis japonica, | |
| | | noroxycodone | Papaver | |
| | | Noroxymorphone→N(cyclopropylmethyl) | somniferum, | |
| | | nornoroxymorphone | Eschscholzia | |
| | | Northebaine→N(cyclopropylmethyl) | californica, Papaver | |
| | | northebaine | bracteatum, | |
| | | Nororipavine→N(cyclopropylmethyl) | Argenome | |
| | | nororipavine | mexicana, Glaucium | |
| | | Norhydrocodone→N(cyclopropylmethyl) | flavum, Sanguinaria | |
| | | norhydrocodone | canadensis, | |
| | | Norhydromorphone→ | Corydalis | |
| | | N(cyclopropylmethyl)norhydromorphone | chelanthifolia, | |
| | | Nordihydrocodeine→ | Nigella sativa, | |
| | | N(cyclopropylmethyl)nordihydrocodeine | Jeffersonia diphylla, | |
| | | Nordihydromorphine→ | Berberis thunbergii, | |
| | | N(cyclopropylmethyl)nordihydromorphine | Mahonia | |
| | | Nor-14-hydroxycodeine→ | aquifolium, | |
| | | N(cyclopropylmethyl)nor-14- | Menispermum | |
| | | | canadense, | |
| | | | Tinospora cordifolia, | |
| | | | Cissampelos | |
| | | | mucronata, | |
| | | | Cocculus trilobus | |

TABLE 12
O-demethylase candidate enzymes

| | | SEQ |
|---|---|---|
| Name | Sequence | ID NO. |
| T6ODM | MEKAKLMKLGNGMEIPSVQELAKLTLAEIPSRYVCANENLLLPMGASVINDHETIPVIDIENLLSPEPIIGKLEL | 83 |
| | DRLHFACKEWGFFQVVNHGVDASLVDSVKSEIQGFFNLSMDEKTKYEQEDGDVEGFGQGFIESEDQTLDWAD | |
| | IFMMFTLPLHLRKPHLFSKLPVPLRETIESYSSEMKKLSMVLFNKMEKALQVQAAEIKGMSEVFIDGTQAMRM | |
| | NYYPPCPQPNLAIGLTSHSDFGGLTILLQINEVEGLQIKREGTWISVKPLPNAFVVNVGDILEIMTNGIYHSVDH | |
| | RAVVNSTNERLSIATFHDPSLESVIGPISSLITPETPALFKSGSTYGDLVEECKTRKLDGKSFLDSMRI | |
| CODM | METPILIKLGNGLSIPSVQELAKLTLAEIPSRYTCTGESPLNNIGASVTDDETVPVIDLQNLLSPEPVVGKLELDK | 84 |
| | LHSACKEWGFFQLVNHGVDALLMDNIKSEIKGFFNLPMNEKTKYGQQDGDFEGFGQPYIESEDQRLDWTEVF | |
| | SMLSLPLHLRKPHLFPELPLPFRETLESYLSKMKKLSTVVFEMLEKSLQLVEIKGMTDLFEDGLQTMRMNYYP | |
| | PCPRPELVLGLTSHSDFSGLTILLQLNEVEGLQIRKEERWISIKPLPDAFIVNVGDILEIMTNGIYRSVEHRAVVN | |
| | STKERLSIATFHDSKLESEIGPISSLVTPETPALFKRGRYEDILKENLSRKLDGKSFLDYMRM | |
| PsP7ODM | MEKAKLMKLGNGLSIPSVQELAELTFAEVPSRYVCTNDENLLLMTMGASEIDDETVPVIDLQNLLSPEPAIGKS | 85 |
| | ELDWLHYSCKEWGFFQLVNHGVDALLVDHVKSEIHSFFNLPLNEKTKYGQRDGDVEGFGQAFLVSENQKLD | |
| | WADMFFINTLPLHLRKPHLFPNLPLPLRETIESYSSEMKKLSMVLFEMMGKAIEVIDIKEAITEMFEDGMQSM | |
| | RMNYYPPCPQPERVIGITPHSDFDGLTILLQLNEVEGLQIRKEDKWISIKPLPDAFIVNVGDIWEIMTNGVHRSV | |
| | DHRGVINSTKERLSIATFHSPKLELEIGPISSLIRPETPAVFKSAGRFEDLLKEGLSRKLDGKSFLDCMRM | |
| PsoDIO | MEKAKLMKLGNGMEIPSVQELAKLTLAEIPSRYVCANENLLLPMGASVINDHETIPVIDIENLLSPEPIIGKLEL | 86 |
| X1 | DRLHFACKEWGFFQVVNHGVDASLVDSVKSEIQGFFNLSMDEKTKYEQEDGDVEGFGQGFIESEDQTLDWAD | |
| | IFMMFTLPLHLRKPHLFSKLPVPLRETIESYSSEMKKLSMVLFNKMEKALQVQAAEIKGMSEVFIDGTQAMRM | |
| | NYYPPCPQPNLAIGLTSHSDFGGLTILLQINEVEGLQIKREGTWISVKPLPNAFVVNVGDILEIMTNGIYHSVD | |
| PsoDIO | METAKLMKLGNGMSIPSVQELAKLTLAEIPSRYICTVENLQLPVGASVIDDHETVPVIDIENLISSEPVTEKLELD | 87 |
| X2 | RLHSACKEWGFFQVVNHGVDTSLVDNVKSDIQGFFNLSMNEKIKYGQKDGDVEGFGQAFVASEDQTLDWADI | |
| | FMILTLPLHLRKPHLFSKLPLPLRETIESYSSEMKKLSMVLFEKMEKALQVQAVEIKEISEVFKDMTQVMRMN | |
| | YYPPCPQPELAIGLTPHSDFGGLTILLQLNEVEGLQIKNEGRWISVKPLPNAFVVNVGDVLEIMTNGMYRSVDH | |
| | RAVVNSTKERLSIATFHDPNLESEIGPISSLITPNTPALFRSGSTYGELVEEFHSRKLDGKSFLDSMRM | |
| PbrDIO | METPKSIKLGGSLLVPSVQELAQQSFAEVPARYVRDDLEPLTDLSGVSMIDQTIPVIDLQKLQSPVPIIRELESEK | 88 |
| X2 | LHSACKEWGFFQVVNHGVDILLVEKTKSEIKDFFNLPMDEKKKFWQEEGDIQGFGQAFVQSEDQKLDWADIF | |
| | LMVTLPRHTRNPRLFPKLPLPLRNTMDSYSSKLSKLASTLIEMMGKALHMETSVLAELFEDGRQTMRINYYPP | |
| | CPQPKDVIGLTPHSDGGGLTILLQLNEVDGLQIRKEKIWIPIKPLPNAFVVNIGNILEIMTNGIYRSVEHRATIHS | |
| | TKERLSVAAFHNPKVGVEIGPIVSMITPESPALFRTIEYDDYGKKYFSRKLDGKSSLDFMRIGEGDEENKAT | |
| PbrDIO | METPKLIKLGGSLLVPSVLELTKQSPAEVPARYIRNDLEPMTDLSSASLTDQTIPVIDLQNLLSPEPELELEKLHS | 89 |
| X3 | GCKEWGFFQVMNHGVDILLVEKVKSEIQGFFNLPIDEKNKFWQEEGDLEGYGKAFVHSEDEKLDWADMFFIL | |
| | TQPQYMRKPRVFPKLPLRLRETIESYSLELSKLGLTLLDLMGKALQIETGVMSELFEDGRQTMRMNYYPPCPQ | |
| | PEHVIGLTPHSDGGALTILLQLNQVDGLQIRKEEIWVPIKPLPNAFVVNIGDILEIMSNGVYRSVEHRATINSSKE | |
| | RLSVAIFQSPKHGTEIGPILSMITPEAPALFKTIPYEDYLRKFFSRKLGGKSFVDSMRIGESDEDNNTA | |
| PbrDIO | METQKQENFGASLSVPNVQELAKQSPEQVPDRYIRSDQDSSTNISCPSMTDQIPVIDLQSLLSPDPIIGELELERL | 90 |
| X4 | HSACKEWGFFQVVNHGVDNLLVEKVKSEIQGFFNLPMDEKKKFWQEEGDFEGFGQAFVFSEDQKLDWGDVF | |
| | FILTQPQHMRKPRLFPKLPLPFRKTIESYSLETNKLSMTLLELMEKALKIETGVMTELFEGGIQRMRMTYYPPC | |
| | PQPKHVIGLTPHSDPDALTILLQLNEVDGLQIRKEKIWVPIKPLSNAFVVNIGDILEIMSNGIYRSVEHRATVNS | |
| | TKERLSVATFHSPRKDTEIGPILITPETPALFRTSGFEDYFRKFFAHKLNGKSFLSSIRIGETDEGNNAT | |
| PbrDIO | MEAPKLIMLGGSLFVPSVQELAKQSLAEVPVRYVRDDQDTLGNNINITPMSMIDQSIPVIDLEKLLSPEPIVGEL | 91 |
| X5 | ELERLHSACKEWGFFQVVNHGVDSLLVEKVKSEIEGFFKLPMDEKTKFWQEEGDIEGFGQVFVHSQDQKLDW | |
| | GDMFLMQTLPRHTRKPRLFPNLPLPLRQTIESYSSELSKLVLTLVDLMGKALQMESGVLTELFENGIQRMRMN | |
| | YYPPCPQPEQVIGLTPHSDVGGLTILLQLNEVDGLQIKKDKVWVPIKPLANAFVVNVGDALEIMSNGIYRSVEH | |
| | RATINSTKERLSIATFHNPRADREIGPIPSMISPETPALFKTTGYEEYFKKFFSRKLEGKSFLDSLRIREGDEHCG | |
| | RLDVKGPCN | |
| PbrDIO | MEIPNPIKIGSSLLVPSVQELAKQSFAEVPARYIRNDVDPLITKLSDVSLIDQTVPVIDLQKLLSPEPIV | 92 |
| X6 | GELELERLHSACKEWGFFQVVNHGVDNLLVEKVKSEIQGFFNLPMEEKKKFWQEEGDFEGFGQMFVQSEE | |
| | QKLDWGDMFFILTQPQHMRKPRLFSKLPLPLRETIESYSLELIKLGLTIIKLMEKALQIDAGVMAELFED | |
| | GIHTMRMNYYPPCPQPEHVIGLTPHSDGGGLTILLQLNEVDGLQIRRENIWVPIKPLPNAFVVNIGDILE | |
| | ILSNGIYRSVEHRSTVNATKERLSVATFQNPKQESVIGPNMITPERPALFRKIVYKDYMKKLFSRKLDGK | |
| | SFLDSLRIGEGDERP | |
| PbrDIO | METLKTVKPGGSLFIPNGQELAKQSLEEVYVGNDQDTMLLIGQTIPVIDLQKLLSPEPITGDMELDKLHS | 93 |
| X8 | ACKEWGFFQVVNHGVDILLVEKVKSEVHDFFNIPMDEKKPFWQEEGDLEGFGQVFITSEDQQLDWGDMFF | |
| | MVTLPKHMRKPRLFLKLPLPLRETIESYSLKLSKLGVTLVELMGKALQMEDRIMSELFDDGRQTMRMNYY | |
| | PPCPQPEQVIGLTPHSDPGGLTILLELNEVNGLIRKENIWVPIIPLPNAFIVNIGDILEIMSNGIYHSVE | |
| | HRATINSTKERLSVAMFNSPKVDTEIGPIHSMITPETPALFRTIGYDEYLKIFFSRKLDGKSLLESMKI | |
| PbrDIO | MEAPKLIMLGGSLFVPSVQELAKQSLAEVPVRYVRDDQDTLGNNINITPMSMIDQSIPVIDLEKLLSPEP | 94 |
| X10 | IVGELELERLHSACKEWGFFQVVNHGVDSLLVEKVKSEIEGFFELPVDEKKKFWQEEGDIEGFGQIFVHS | |
| | EDQKLDWADMFYMLTLPPNMRKPRLFPNLPLPLRQTIDSYSSELSKLVLTLVDLMGKALQMESGVLTELF | |
| | ENGIQRMRMNYYPPCPQPEQVIGLTPHSDVGGLTILLQLNEVDGLQIKKDKIWVPIKPLRNAFVVNVGDA | |
| | LEIMSNGIYRSVEHRATINSTKERLSIATFHNPRADREIGPIPSMISPETPALFKTTGYEEYFKKFFSRK | |
| | LEGKSFLDSLRIGEGDEHCGRLXVKGXCN | |
| PbrDIO | METPKLMKLGGSLFVPSVQELAKQSLAEVPARYVRDDRDMVGNIINVTPMSMIDQSIPVIDLEKLLSPDL | 95 |
| X11 | IVGELELERLHSACKEWGFFQVVNHGVDSLLVEKVKSEIEGFFELPMDEKKKFWQEEGDAEGFAQFFVQS | |
| | EDQKLDYSGDMFFMLNLPQHMRKPRLFLKLPLPLRETIESYSLKLSKLGVTLVELMGKALQMEDRIMSEL | |
| | FDDGRQTMRMNYYPPCPQPEQVIGLTPHSDPGGLTILLELNEVNGLIRKENIWVPIIPLPNAFIVNIGDI | |
| | LEIMSNGIYHSVEHRATINSTKERLSVAMFNSPKVDTEIGPIHSMITPETPALFRTIGYDEYLKIFFSRK | |
| | LDGKSLLESMKI | |
| PbrDIO | METPKLRDFGSFLPVPSVQELAKQVLTEIPPRYIRTDLEALNKLSCASNTDQTVPIIDMQCLLSAEPEME | 96 |
| X13 | LEKLHSACKEWGFFRVVNHGVDNLESVKSEIESFLNLPVNAKNKYGQKQGDDQGFGSRFVLSEEQKLDWG | |
| | DFFYMVTRPLYLRKPHLFPELPLPLRETIESYSSEVSKLAMALFEMMGKALKIETGVMTEIFEGGMQAMR | |
| | MNYYPPCPRPDLVIGLNAHSDFGGLTILLQLNEVEGLEIRNKGEWVSVKPLANAFVVNVGDVMEILTNGI | |
| | YHSVEHRATINSSKERLSVATFHYPKLETGIGPLPCMITPKTPALFGRIERYELLLRKYYARKLNGKSTL | |
| | DCMRIGNGFEDDNTA | |
| PbrDIO | MEAPKLIMLGGSLFVPSVQELAKQSLAEVPARYVRDDQDTLGNNINITPMSMIDQSIPVIDLEKLLSPEP | 97 |
| X18 | IVGELELERLHSACKEWGFFQVVNHGVDSLLVEKVKSEIEGFFELPVDEKKKFWQEEGDIEGFGQIFVHS | |
| | EDQKLDWADMFYMLTLPPNMRKPRLFPNLPLPLRQTIDSYSSELSKLVLTLVDLMGKALQMESGVLTELF | |
| | ENGIQRMRMNYYPPCPQPEQVIGLTPHSEVGGLTILLQLNEVDGLQIRKEKIWVPIKPLSNAFIVNIGDI | |
| | LEIMSNGIYRSVEHRATVNSTKERLSVATFHSPRKDTEIGPILITPETPALFRTSGFEDYFRKFFAHKLN | |
| | GKSFLSSIRIGETDEGNNAT | |
| PbrDIO | MSMIDQSIPVIDLEKLLSPEPIVGELELERLHSACKEWGFFQVVNHGVDSLLVEKVKSEIEGFFELPVDE | 98 |
| X19 | KKKFWQEEGDIEGFGQIFVHSEDQKLDWADMFYMLTLPPNMRKPRLFPNLPLPLRQTIDSYSSELSKLVL | |
| | TLVDLMGKALQMESGVLTELFENGIQRMRMNYYPPCPQPEQVIGLTPHSDVGGLTILLQLNEVDGLQIRK | |
| | EKIWVPIKPLSNAFIVNIGDILEIMSNGIYHSVEHRATINSTKERLSVAMFNSPKVDTEIGPIHSMITPE | |
| | TPALFRTIGYDEYLKIFFSRKLDGKSLLESMKI | |
| PbrDIO | METPKLVKSSGSSLFLSTSVQELAKQSLPEVPARYIRTNLEPLSNVSGDSQSVPVIDLQKLLSSEPIIGE | 99 |
| X21 | LELDKLHSACKEWGFFQVVNHGVDNLVMEKIKTEIQGFFNLSLDEKQKFWKKEGDAEGFGQNFIESEDQK | |
| | LDWGDTFGMFTLPIHMRNPRLFPELPLPLRETIESYSLDVRKLALALIGLMEKALKIKTSAMSELFEDGG | |
| | QAMRMNYYPPCPQPEHVIGLTPHSDAGGLTILLQLNEVDGLQIKKDKIWVPIKPLPNAFVVNIGDILEIM | |
| | TNGIYRSVEHRATINSSKERLSVAAFHSPKGDTLIGPMVSLITPETPALFRTIGYQDYMKKFMSRKLDGK | |
| | SLVNSMRIGEGDEDK | |
| PbrDIO | METPTLMKLGNGLSVPSVQELAKATLAEIPSRYICTDENLLTMGASTTDNETVPVIDLQNLLSPEPVIGMLELD | 100 |
| X-ZSNV- | RLHSACKEWGFFQLVNHGVDALLVDNEVQGFFNLPMDEKTKYGQKDGDDEGFGQFFVISEDQKLDWADVFY | |
| 2004018 | MSTLPLHSRKPHLFPELPLPLRETMESYSSEMKKLSMVLFDMMGKALQVVEIKGITELFEDGAQQIRMNYYPP | |
| | CPQPELVFGLTSHSDFDGLTILLQLGEVEGLQIKKEERWISIKPLPDAFIVNVGDILEIMTNGIYRSVDHRAVVNS | |
| | IKERLTIATFHDPRLEAEIGPISSLITPETPALFKRGVFEDLLKEMFLRKLDGKSFLDCMRM | |
| PrhDIO | GNGLSVPSVQELAKQTLAEIPSRYICTDENPLITGASVVDDETVPVINLQNLLSPEPVIGKLELDKLHSACKEWG | 101 |
| X-MVTX- | FFQVVNHGVNDSLVDSVKSEIEGFFNLPANEKLKYGQKDGDVEGFGQHFVVSEDQKLDWADVFYMVTLPVRL | |
| 2001522 | RKPHLFPELPLPLRDTLDSYSSELNKLSMVLLEMMEKALKLVECKGITDFFEDGFQQMRMNYYPPCPRPELVT | |
| | GLTSHSDFGGLTILLQLNDVEGLQIKKEERWISIKPLPNAFIVNIGDVLEIMSNGIYRSVDHRAVINSTKVRMSVA | |
| | TFHDPRLEAVIGPISSLITPETPALFKRGVFEDLLKEMFLRKLDGKSFLDCMRI | |
| PseDIO | LMKLANGMSVPIVQELAKLTVGEIPSRYICTDGNLLTMGASVIDYETVPVIDLQNLQSREPVIEKLELDRLHSAC | 102 |
| X-JSVC- | KEWGFFQLLNHGVDASLMDNVRSEIRGFFNLPISDKMKYGQKDGDEEGFGQHFIVSEDQKLDWVDAFMMFT | |
| 2005842 | LPLHSRNPRLTPEFPQPLRETVESYSSEMKKLSVLLFELMEKALQVKGITEMFEDGLQSIRMNYYPPCPRPELA | |
| | IGLTSHSDFDGLTILLQLNEVEGLQIKKEERWISIKPLPNAFIVNVGDVLEVMTNGIYRSVDHRAVVNSTKERLS | |
| | IATFHDPELESEIGPIASLITPETPALFKRGRFKDLLKENLSTKLDGKSFLDCIRM | |
| CYP2D6 | MGLEALVPLAVIVAIFLLLVDLMHRRQRWAARYSPGPLPLPGLGNLLHVDFQNTPYCFDQLRRRFGDVFSLQL | 103 |
| | AWTPVVVLNGLAAVREALVTHGEDTADRPPVPITQILGFGPRSQGVFLARYGPAWREQRRFSVSTLRNLGLGK | |
| | KSLEQWVTEEAACLCAAFANHSGRPFRPNGLLDKAVSNVIASLTCGRRFEYDDPRFLRLLDLAQEGLKEESGF | |
| | LREVLNAVPVLLHIPALAGKVLRFQKAFLTQLDELLTEHRMTWDPAQPPRDLTEAFLAEMEKAKGNPESSFN | |
| | DENLRIVVADLFSAGMVTTSTTLAWGLLLMILHPDVQRRVQQEIDDVIGQVRRPEMGDQAHMPYTTAVIHEV | |
| | QRFGDIVPLGVTHMTSRDIEVQGFRIPKGTTLITNLSSVLKDEAVWEKPFRFHPEHFLDAQGHFVKPEAFLPFS | |
| | AGRRACLGEPLARMELFLFFTSLLQHFSFSVPTGQPRPSHHGVFAFLVTPSPYELCAVPR | |

TABLE 13
Tailoring enzymes

| Reaction Catalyzed | Enzyme | Species |
|---|---|---|
| Carbon-carbon | Berberine bridge enzyme (BBE) | Ps, Ec, Cj, Bs, Tf |
| coupling | Salutaridine synthase (SalSyn) | Ps |
| | Corytuberine synthase (CorSyn) | Cj |
| Oxidation | Tetrahydroprotoberberine oxidase (STOX) | Cj, Am, Bw |
| | Dihydrobenzophenanthridine oxidase | Ps |
| | (DBOX) | |
| Methylenedioxy | Methylstylopine hydroxylase (MSH) | Ps |
| bridge | Protopine 6-hydroxylase (P6H) | Ps, Ec |
| formation | Stylopine synthase (StySyn) | Ps, Ec, Am |
| | Cheilanthifoline synthase (CheSyn) | Ps, Ec, Am |
| | Canadine synthase (CAS) | Tf, Cc |
| O-methylation | Norcoclaurine 6-O-methyltransferase | Ps, Tf, Cj, Pb |
| | (6OMT) | |
| | 3′hydroxy-N-methylcoclaurine 4′-O- | Ps, Tf, Cj, Cc |
| | methyltransferase (4′OMT) | |
| | Reticuline 7-O-methyltransferase (7OMT) | Ps, Ec |
| | Scoulerine 9-O-methyltransferase (9OMT) | Ps, Tf, Cj, Cc |
| N-methylation | Coclaurine N-methyltransferase (CNMT) | Ps, Tf, Cj |
| | Tetrahydroprotoberberine N- | Ps, Ec, Pb |
| | methyltransferase (TMNT) | |
| O-demethylation | Thebaine demethylase (T6ODM) | Ps |
| | Codeine demethylase (CODM) | Ps, Ga |
| Reduction | Salutaridine reductase (SalR) | Ps, Pb, Ga |
| | Codeinone reductase (COR) | Ps |
| | Sanguinarine reductase (SanR) | Ec |
| Acetylation | Salutaridine acetyltransferase (SalAT) | Ps |

TABLE 14
Comparison of impurities that may be present in concentrate
of poppy straw and clarified yeast culture medium.

| | | Concentrate | |
|---|---|---|---|
| | | of | Clarified Yeast |
| Impurities: | | Poppy Straw | Culture Medium |
| Inorganic | Sodium | ✓ | ✓ |
| | Magnesium | ✓ | ✓ |
| | Silicon | ✓ | x(not in culture medium) |
| | Phosphorus | ✓ | ✓ |
| | Sulfur | ✓ | ✓ |
| | Chloride | ✓ | ✓ |
| | Potassium | ✓ | ✓ |
| | Calcium | ✓ | ✓ |
| | Copper | ✓ | ✓ |
| | Zinc | ✓ | ✓ |
| | Molybdenum | ✓ | ✓ (sodium molybdenite in |
| | | | medium) |
| | Iron | ✓ | ✓ |
| | Manganese | ✓ | ✓ |
| | Ammonium | ✓ | ✓ |
| | Boron | ✓ | ✓ |
| Organic | Polysaccharides (starch, cellulose, xylan) | ✓ | x(yeast fed simple sugars) |
| | Lignin (p- cournaryl,, coniferyl, sinapyl, alcohols) | ✓ | x |
| | Pigments (chlorophyll, anthocyanins, carotenoids) | ✓ | x |
| | Flavonoids | ✓ | x |
| | Phenanthreoids | ✓ | x |
| | Latex, gum, and wax | ✓ | x |
| | Rubisco | ✓ | x |
| | Meconic acid | ✓ | x |
| | Pseudomorphine | ✓ | x |
| | Narceine | ✓ | x |
| | Thebaol | ✓ | x |
| Other | Pesticides | ✓ | x |
| | Pollen | ✓ | x |

TABLE 15
Distinct groups of molecules present in clarified yeast culture medium (CYCM). Unlike concentrate of poppy
straw (CPS), yeast host strains may be engineered to produce molecules of a predetermined class of alkaloids
(i.e., only one biosynthesis pathway per strain) such that other classes of alkaloids are not present. Therefore,
the CYCM may contain molecules within a single biosynthesis pathway including a subset of molecules spanning
one or two columns, whereas the CPS may contain a subset of molecules across many columns.

| 1- | Protoberberine and | | | | |
|---|---|---|---|---|---|
| Benzylisoquinoline | Phthalideisoquinoline | Morphinan | Isopavine | Aporphine | BisBIA |
| Tetrahydropapaverine | Scoulerine | Salutaridine | Pavine | Magnoflorine | Dauricine |
| Dihydropapaverine | Chelanthifoline | Salutaridinol | Caryachine | Corytuberine | Berbamunine |
| Papaverine | Stylopine | Salutaridine-7-O-acetate | Bisnorargemonine | Apomorphine | Ligensinine |
| | Cis-N-methylstylopine | Thebaine | Isonoraremonine | Boldine | Fangchinoline |
| | Protophine | Codeinone | | | Tetrandrine |
| | Dihydrosanguinarine | Oripavine | | | Curine |
| | Sanguinarine | Morphinone | | | Cepharanthine |
| | Tetrahydrocolumbamine | Neopinone | | | Berbamine |
| | Canadine | Neopine | | | |
| | N-methylcanadine | Codeine | | | |
| | Noscapine | Morphine | | | |
| | Berberine | Neomorphine | | | |
| | | Hydrocodone | | | |
| | | Oxycodone | | | |
| | | 14-hydroxycodenone | | | |
| | | 14-hydroxycodeine | | | |
| | | Dihydromorphine | | | |
| | | Dihydrocodeine | | | |

TABLE 16
Impurities that may be present in chemical synthesis preparations of compounds

| Compound | Impurities |
|---|---|
| Buprenorphine | 15,16-Dehydrobuprenorphine, 17,18-Dehydrobuprenorphine, 18,19- |
| | demethylbuprenorphine, 19,19′-Ethylbuprenorphine, 2,2′-Bisbuprenorphine, |
| | 3-Deshydroxybuprenorphine, 3-O-Methylbuprenorphine, 3-O-Methyl-N- |
| | cyanonorbuprenorphine, 3-O-Methyl-N-methylnorbuprenorphine, 6-O- |
| | Desmethylbuprenorphine, Buprenorphine N-oxide, N-But-3- |
| | enylnorbuprenorphine, N-But-3-enylnormethylbuprenorphine, N- |
| | Butylnorbuprenorphine, N-Methylbuprenorphine, Norbuprenorphine, |
| | Tetramethylfuran buprenorphine |
| Oxymorphone | 1-Bromooxymorphone, 6-Beta oxymorphol, 10-Alpha-hydroxyoxymorphone, |
| | 10-Ketooxymorphone, 2,2-Bisoxymorphone, Noroxymorphone, |
| | Oxymorphone N-oxide, 10-Hydroxyoxymorphone, 4-Hydroxyoxymorphone, |
| | 8-Hydroxyoxymorphone, Hydromorphinol. |
| Naltrexone | 10-Hydroxynaltrexone, 10-Ketonaltrexone, 14-Hydroxy-17- |
| | cyclopropylmethylnormorphinone, 2,2′-Bisnaltrexone, 3- |
| | Cyclopropylmethylnaltrexone, 3-O-Methylnaltrexone, 8-Hydroxynaltrexone, |
| | N-(3-Butenyl)-noroxymorphone, Naltrexone aldol dimer, N-Formyl- |
| | noroxymorphone |
| Naloxone | 10-Alpha-hydroxynaloxone, 10-Beta-hydroxynaloxone, 10-Ketonaloxone, 3- |
| | O-Allylnaloxone, 7,8-Didehydronaloxone, 2,2′-Bisnaloxone, Naloxone N- |
| | oxide |
| Nalbuphine | Beta-epimer of nalbuphine, 2,2′-Bisnalbuphine, 6-Ketonalbuphine, 10- |
| | Ketonalbuphine, Alpha-noroxymorphol, N-(Cyclobutylcarbonyl)-alpha- |
| | noroxymorphol, N-Formyl-6-alpha-noroxymophol. |

TABLE 17
P450 sequences.

| SEQ ID | | |
|---|---|---|
| NO. | Description | Sequence |
| 104 | Sorangium | MVDQDAFPELFHPSSRAEPHAIYARMRAAGRLHRLVHPRLDVPIWVAVRY |
| | cellulosum | DDCVELLKDPRLIRDFRKLPDEVRRRYFPLSDRTTFMDQHMLDADPPDHT |
| | species of | RLRAIVQRAFSPRMMEGLRPRIQEIADGLIDAVIDRRRMELIADFAFPLPTAV |
| | origin; amino | IAELLGLPVEDRGRFRRWTKILLAPAKDREFVERAQPVVEEFAAYFRALADA |
| | acid sequence; | RRKAPRDDLISGLLLAEEQEHKLSPAELSSMVFLLLVAGHETTVHLIASGMLL |
| | 14HC_P450_1 | LLSHPAERRRLDEDPGLVGSAVEEALRCEGPAELSTIRWSLEDIELFGARVPA |
| | | GEGVAAGLLAANRDPQHFPDPDRFDIGRSPNRHIGFGGGIHFCLGAMLARIE |
| | | AAIAFSTLLRRLPRIELATSTRDIVWSEWPTIRGPAAVPVVF* |
| 105 | Sorangium | ATGGTGGACCAGGATGCTTTTCCTGAGCTTTTTCATCCCAGCTCACGTGCCG |
| | cellulosum | AGCCCCATGCGATATACGCCAGGATGAGAGCGGCTGGGAGGTTGCACAGAC |
| | species of | TAGTGCATCCTAGGCTTGATGTGCCGATCTGGGTTGCAGTACGTTATGACG |
| | origin; nucleic | ACTGCGTGGAGCTATTGAAAGATCCTAGACTAATTCGTGATTTTCGTAAAT |
| | acid sequence | TACCCGACGAAGTCCGTCGTAGATACTTTCCGTTAAGTGATCGTACGACTT |
| | 14HC_P450_1 | TCATGGACCAACATATGTTGGATGCTGATCCCCCGGACCATACCAGACTGA |
| | | GAGCAATCGTACAAAGAGCGTTCTCCCCGAGAATGATGGAGGGCTTACGTC |
| | | CTCGTATTCAAGAAATCGCAGATGGACTAATAGATGCTGTAATTGATCGTA |
| | | GAAGGATGGAGCTTATTGCGGACTTTGCGTTCCCACTTCCCACGGCCGTTA |
| | | TTGCGGAACTGTTGGGCCTACCTGTGGAGGATAGGGGCAGATTCCGTCGTT |
| | | GGACTAAAATTCTTCTAGCTCCTGCGAAGGATCGTGAGTTCGTAGAGAGAG |
| | | CCCAACCAGTGGTTGAGGAGTTTGCCGCCTACTTTCGTGCGTTGGCAGACG |
| | | CTAGGAGGAAGGCACCTCGTGATGATCTGATCTCCGGACTGCTTCTGGCAG |
| | | AAGAACAGGAGCATAAACTAAGCCCAGCGGAATTAAGTAGCATGGTATTCC |
| | | TTCTGTTAGTTGCAGGTCATGAAACGACCGTACACTTAATCGCGTCAGGCA |
| | | TGCTTCTATTGCTTTCTCACCCGGCTGAAAGGAGAAGGTTGGACGAGGACC |
| | | CAGGCCTAGTTGGGTCAGCCGTCGAGGAGGCGTTACGTTGTGAGGGTCCAG |
| | | CAGAGTTGTCCACTATCAGGTGGTCACTTGAGGATATAGAGTTGTTCGGCG |
| | | CCAGAGTGCCTGCCGGCGAGGGGGTTGCAGCTGGCTTACTAGCGGCTAATC |
| | | GTGACCCCCAACATTTCCCAGACCCCGATAGATTTGATATTGGGAGATCAC |
| | | CTAATAGACACATCGGTTTTGGCGGGGGAATACACTTTTGTCTTGGTGCTA |
| | | TGTTGGCTAGGATAGAAGCCGCAATCGCTTTCAGTACGTTATTAAGGCGTT |
| | | TGCCCCGTATTGAGCTTGCAACATCTACCCGTGATATAGTTTGGAGCGAGT |
| | | GGCCCACTATCCGTGGGCCCGCAGCGGTTCCGGTGGTTTTTTGA |
| 106 | Absidia glauca | MPIVNALQTYSYEEPINFLKDVWLNRLFPAGTSNRKKAALLTLGATIALVCR |
| | species of | TVYRLYRVPRSLRHIPAVGYVAMLRSILKREDATTRAMTIFQPAMKKGNGV |
| | origin; amino | FLVNFPLEWSIYVAEPLAAKAVLMKSDNFPKSIDFIHALGKENPVVKFFGTD |
| | acid sequence; | NVALVNGEQWKRQRKVMNPAFHRAMPVQMFGRLLQKGFKNIEEQGHQVA |
| | 14HC_P450_2 | ALDFFQRLTLDALGHAVFGFDFGALNDREAVWTVTYESIRLNLRNPLAFAF |
| | | PSMDWLLKYVIPGRLQMAASVDKLNGLMMDMIKEKRIKLLESQAQDDTPE |
| | | NEKDLLTLMLEAEQRGEGTTNDEELRSNMAVFFLAGHETTANTMSFCLYN |
| | | LAMDKSVQEKARQEVIKVLGDEPEDVLPRNEELRQIPYLDMILKENLRRFGP |
| | | ASMLNARKTVEDFDMNGTFIPKNTSVIVETNALHHNPEVWKNPEQFDPER |
| | | FAPGGEHETSHEGMAWLPFSSGSRGCLGMNFSMAEQRVFLAMLLRKYEWD |
| | | LPKDSIHKNGIQIGNFQNACPDSLKIQFTPRY* |
| 107 | Absidia glauca | ATGCCTATAGTAAACGCCCTTCAGACATATTCATATGAGGAACCAATCAAC |
| | species of | TTTCTTAAGGATGTATGGTTAAACCGTCTGTTCCCGGCTGGCACATCCAAC |
| | origin; nucleic | AGGAAGAAAGCAGCACTACTAACGCTAGGCGCGACTATCGCGTTGGTATGT |
| | acid sequence; | AGAACGGTGTACAGGTTGTATCGTGTTCCGCGTAGTTTAAGGCACATCCCA |
| | 14HC_P450_2 | GCGGTTGGATACGTGGCTATGCTAAGAAGTATTCTTAAGCGTGAGGATGCG |
| | | ACAACCAGGGCTATGACGATTTTTCAACCCGCCATGAAAAAGGGTAATGGG |
| | | GTTTTCCTAGTAAATTTCCCCTTGGAATGGTCTATTTATGTGGCAGAACCC |
| | | TTAGCTGCTAAAGCCGTCTTGATGAAGTCCGACAATTTCCCGAAATCCATT |
| | | GACTTCATCCATGCGTTGGGTAAGGAGAACCCAGTCGTTAAGTTTTTCGGC |
| | | ACCGATAACGTGGCACTTGTCAACGGGGAACAGTGGAAAAGGCAGAGGAAA |
| | | GTTATGAACCCTGCATTTCATAGAGCCATGCCAGTACAGATGTTCGGCAGG |
| | | TTGTTGCAGAAAGGATTTAAAAATATTGAGGAACAAGGCCACCAAGTGGCG |
| | | GCCTTAGACTTTTTTCAACGTCTTACGCTGGATGCGTTAGGACATGCTGTG |
| | | TTCGGGTTCGACTTCGGGGCTTTGAATGATCGTGAAGCTGTGTGGACGGTA |
| | | ACTTATGAGTCAATCAGACTGAACCTAAGAAATCCTCTGGCATTTGCGTTC |
| | | CCTTCCATGGACTGGCTTCTTAAATATGTGATTCCCGGGAGATTACAGATG |
| | | GCAGCGTCCGTAGACAAGTTGAATGGTCTAATGATGGATATGATAAAAGA |
| | | AAAAAGGATTAAGTTGCTAGAGTCACAGGCTCAGGACGACACTCCGGAGAA |
| | | TGAAAAAGATTTGCTTACGCTGATGCTGGAAGCGGAACAGAGAGGCGAGGG |
| | | AACCACTAACGACGAAGAACTTAGAAGCAATATGGCCGTCTTTTTCTTGGC |
| | | TGGACATGAGACAACCGCCAATACCATGTCCTTCTGCTTATACAATTTAGC |
| | | AATGGACAAGAGTGTGCAGGAGAAGGCGAGACAGGAGGTAATAAAGGTCC |
| | | TAGGGGATGAGCCAGAGGATGTATTGCCACGTAACGAAGAGCTAAGACAAA |
| | | TCCCGTATCTAGATATGATATTAAAGGAGAATCTTCGTAGGTTCGGTCCTG |
| | | CGTCTATGCTTAATGCCAGGAAGACCGTGGAAGACTTTGATATGAATGGCA |
| | | CCTTCATCCCGAAGAATACGTCTGTAATTGTCGAAACAAACGCGCTTCATC |
| | | ACAATCCTGAAGTATGGAAGAACCCGGAGCAATTCGATCCGGAAAGATTTG |
| | | CTCCAGGTGGAGAGCACGAGACCAGCCACGAAGGGATGGCTTGGTTGCCTT |
| | | TTTCATCTGGTAGCAGGGGTTGTTTGGGAATGAACTTTTCTATGGCGGAAC |
| | | AGCGTGTCTTCTTGGCAATGCTGCTGAGGAAATACGAGTGGGATCTACCAA |
| | | AGGATAGCATTCATAAGAACGGTATCCAGATTGGTAACTTTCAAAACGCTT |
| | | GCCCTGACAGTCTGAAGATCCAATTTACGCCGCGTTATTAG |
| 108 | Choanephora | MFTETALQIYHQMAEKVLPILKRQPKSSYIGAALVFLLVAEIRRQLSVPKHLK |
| | cucurbitarum | KFPTIGVFTLMKSFMRNDSVIERHNRLVAPIVKQGHKFYAAKIPFDWSLSIV |
| | species of | DPEIAKIMLMKSDAFPKSQGFTRKLGDSSLIVRFTGRDNVSISNGHVWKRQR |
| | origin; amino | KFMNPAFRRSTPIKTFGELTLKFFDCVDEQPQDFPAAIKLKNYTLDALGIAA |
| | acid sequence; | FDFDFKSLSGDPEGWTEIYNVIMKGMFDPWVFLFGKMEFILQYIIPSKRECIK |
| | 14HC_P450_3 | SVVKFNKMLVEMADKRRQEIQNGKKLNTPNSEKDLLTLMIEAEMEEGIMT |
| | | TNEELRENIALFFLAGQDSTGNSLSFCLYHLAKNKHVQDKLRREIMSVMGD |
| | | RDLDAIPTVEDFKDMPYLNMVIKENLRLSGPADRFLDRVVAEDIVLGGELIP |
| | | KGTLITVDVASIHYNPEYWHDPEVFIPERFEPNGEFDQHAGVAWLPFSNGA |
| | | RQCIGMNFSLAEQRVFLTMLLRRYEVGISKDSIHYDSIVYEQSFTFAPSSLTLN |
| | | FTKLN* |
| 109 | Choanephora | ATGTTCACAGAGACTGCGTTGCAGATCTATCACCAAATGGCGGAAAAAGTG |
| | cucurbitarum | CTACCCATTTTGAAAAGACAACCCAAATCTTCTTATATCGGCGCGGCGTTA |
| | species of | GTCTTCCTGCTTGTAGCCGAAATAAGAAGGCAGCTATCTGTACCTAAACAC |
| | origin; nucleic | CTGAAGAAGTTTCCGACGATTGGGGTTTTCACGTTAATGAAGAGCTTTATG |
| | acid sequence; | AGGAATGACTCAGTGATCGAAAGGCATAATAGGTTGGTGGCACCCATTGTG |
| | 14HC_P450_3 | AAGCAAGGTCATAAGTTTTACGCGGCAAAGATTCCTTTTGATTGGTCTTTG |
| | | TCAATTGTGGACCCAGAGATCGCCAAGATAATGCTTATGAAATCTGATGCT |
| | | TTCCCCAAAAGCCAGGGTTTCACCAGGAAACTGGGTGACTCATCACTGATC |
| | | GTGAGGTTTACTGGCAGGGATAATGTGAGTATTTCTAATGGACACGTTTGG |
| | | AAAAGACAGAGAAAGTTCATGAATCCCGCGTTCAGGCGTTCTACACCCATT |
| | | AAGACCTTCGGGGAATTAACGCTAAAATTCTTCGATTGTGTAGATGAACAA |
| | | CCGCAAGATTTCCCTGCCGCAATTAAACTAAAGAACTACACACTTGATGCG |
| | | CTGGGAATTGCGGCATTCGATTTCGACTTTAAAAGTCTATCTGGCGATCCC |
| | | GAAGGATGGACAGAAATTTACAACGTGATCATGAAGGGTATGTTTGATCCA |
| | | TGGGTATTCTTGTTCGGTAAGATGGAGTTTATTCTACAGTATATCATACCA |
| | | TCAAAACGTGAGTGCATTAAATCTGTCGTTAAGTTCAATAAAATGCTAGTC |
| | | GAAATGGCAGACAAAAGACGTCAAGAAATCCAGAATGGAAAAAAATTGAA |
| | | CACGCCCAATAGTGAAAAAGACCTGCTAACTTTGATGATAGAAGCGGAAAT |
| | | GGAAGAGGGTATAATGACTACTAATGAAGAGCTTCGTGAGAACATCGCCCT |
| | | ATTTTTCTTGGCCGGTCAGGACAGTACTGGTAACAGTCTGAGTTTCTGTTT |
| | | ATATCATTTGGCAAAAAATAAACATGTACAGGACAAACTAAGAAGGGAGA |
| | | TCATGTCTGTGATGGGGGATAGAGACCTTGATGCAATACCCACGGTGGAAG |
| | | ACTTCAAAGACATGCCTTATCTTAATATGGTAATCAAAGAAAACTTACGTT |
| | | TGTCTGGCCCTGCGGATCGTTTCCTAGATAGAGTTGTAGCGGAAGATATCG |
| | | TGCTTGGGGGTGAGTTAATCCCAAAAGGAACCTTAATAACAGTTGATGTAG |
| | | CCAGTATTCATTACAATCCTGAGTACTGGCATGACCCGGAGGTCTTCATCC |
| | | CCGAAAGATTCGAGCCGAACGGTGAGTTCGATCAGCACGCTGGTGTGGCGT |
| | | GGCTACCATTCTCTAATGGAGCCCGTCAATGCATAGGTATGAATTTCAGTC |
| | | TTGCTGAGCAGAGAGTGTTTCTGACTATGTTATTGCGTCGTTACGAGGTGG |
| | | GTATCAGTAAAGATAGCATCCATTACGATAGCATAGTATATGAGCAATCAT |
| | | TTACGTTTGCTCCTAGTAGCCTTACGCTGAATTTTACGAAGCTGAACTAG |
| 110 | Lichtheimia | MYTLVSFLKDQNIIAVLKNAIHAQQQGTTASSITILSLAVAITTFAVHRIRLYS |
| | corymbifera | VKEHGVPLVPYVLPFIGSSPEYRKDPKAFLEKWTAKFGPVFRAHIFGRVYTII |
| | species of | SGHYVREVFLNEDFSFEVGMGKTFDGWLITDTKKSELFSTPLVRSMVMKHL |
| | origin; amino | AVDVKNYTPRAVEHLTIAAREMLGDIGESKELPHLYPLIQHMVSTAIASILVG |
| | acid sequence; | LKICKDKDLLETFKNVAVDIGSELNPDSYLYEAFPTISRLRQWYLGKYGKAIN |
| | 14HC_P450_4 | KHQEHMLRVLGPEIDERLAAMERGDGGWERPEDILQGILETAKLTSDHPQR |
| | | YMVPIKWFLILVFASIHTTSQNTTVAMYRLLQHPEVIDELLEEQNQVFEKHH |
| | | GSNYDDHDITKLLTGEVIKDLVKLDSVCREAMRISSFYAELPHTYIGKSPLTM |
| | | SNGTIINPGDDVLINGYTNHHDPDIQIDGGGDYAEFKPFRFVEKGRQSTRIGD |
| | | DYLIFGQGKHACPGRWFAMQEMKTTISFLIRQYIITAKGDIMFLKGHRQKIP |
| | | MGQVIFQKR* |
| 111 | Lichtheimia | ATGTATACCCTAGTCTCATTCTTAAAAGACCAAAACATCATTGCGGTGTTG |
| | corymbifera | AAAAATGCCATTCATGCACAACAACAAGGCACCACCGCAAGTTCTATTACT |
| | species of | ATTCTTTCTCTAGCTGTTGCGATAACGACTTTCGCTGTTCATCGTATTAGA |
| | origin; nucleic | CTTTACTCCGTTAAGGAGCATGGTGTTCCCCTGGTACCGTATGTCCTACCCT |
| | acid sequence; | TTATTGGGTCCTCTCCAGAGTACAGAAAAGACCCAAAGGCCTTCCTAGAAA |
| | 14HC_P450_4 | AGTGGACCGCTAAATTCGGGCCAGTGTTCCGTGCGCATATCTTTGGCAGGG |
| | | TGTACACAATAATATCCGGCCATTACGTCAGGGAGGTTTTCTTGAATGAAG |
| | | ATTTCAGCTTTGAAGTTGGTATGGGTAAAACGTTCGACGGCTGGCTTATAA |
| | | CCGACACAAAGAAATCTGAGCTGTTTTCCACTCCACTTGTACGTAGTATGG |
| | | TCATGAAGCATCTGGCCGTTGATGTCAAGAACTACACGCCGAGAGCAGTGG |
| | | AACATCTTACCATTGCCGCTAGGGAGATGCTGGGTGACATCGGTGAATCAA |
| | | AAGAGCTGCCACACTTATATCCCCTGATTCAACATATGGTAAGCACGGCGA |
| | | TAGCCTCTATCTTGGTGGGCCTTAAAATCTGTAAAGACAAAGATCTACTTG |
| | | AGACCTTTAAGAACGTGGCCGTTGATATAGGCTCTGAATTGAATCCAGACT |
| | | CCTACTTGTATGAAGCGTTTCCTACCATAAGCAGGTTGAGACAGTGGTACC |
| | | TGGGAAAGTATGGGAAGGCAATCAACAAACACCAGGAACACATGCTGAGAG |
| | | TACTGGGACCTGAGATAGATGAACGTCTAGCGGCCATGGAGCGTGGAGATG |
| | | GTGGCTGGGAAAGGCCCGAGGACATTCTTCAAGGGATTTTAGAGACCGCCA |
| | | AACTGACAAGTGATCACCCTCAACGTTACATGGTTCCCATCAAATGGTTTC |
| | | TGATATTGGTTTTTGCAAGTATCCACACGACCTCACAGAATACAACGGTAG |
| | | CTATGTACAGGCTTTTGCAACATCCGGAAGTTATAGATGAGTTACTTGAAG |
| | | AACAAAACCAGGTCTTCGAGAAGCACCACGGGAGCAATTATGATGATCATG |
| | | ACATAACTAAGTTGCTAACGGGGGAAGTCATAAAAGATTTGGTCAAGTTAG |
| | | ACTCAGTCTGCCGTGAAGCAATGCGTATATCATCTTTCTACGCTGAATTGC |
| | | CTCACACATACATTGGCAAGAGTCCATTAACTATGAGTAACGGAACAATCA |
| | | TCAACCCCGGAGACGATGTGCTGATTAATGGCTACACCAACCACCATGACC |
| | | CCGACATTCAGATTGACGGGGGTGGCGACTATGCAGAATTCAAGCCCTTCA |
| | | GGTTTGTCGAGAAAGGCAGGCAGTCTACAAGAATCGGAGACGATTATCTTA |
| | | TCTTCGGACAAGGCAAACACGCATGTCCAGGAAGGTGGTTTGCCATGCAAG |
| | | AAATGAAGACTACTATCTCCTTCTTGATACGTCAATATATTATCACAGCCA |
| | | AGGGTGATATAATGTTCCTAAAGGGTCACAGACAAAAAATCCCTATGGGAC |
| | | AGGTGATTTTTCAGAAGCGTTAG |
| 112 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTLDALGKSVFGFEFGALDDPDS |
| | 14HC_P450_5 | VWVKTYRQVFDSFTDVFSLVFPRLDPIYRYFSAKHREQYNAVYKLIDLLDGM |
| | | ADKKRSMLQDASNSDIKDVPDHEKDLLQLMLEAELRGEGSWTKRELRHNM |
| | | AIFFVAGQDTTSHALTFCLYLLAKNQDIQKKAREEILNVFGDEPKDVFPTLE |
| | | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 113 | Lichtheimia | ATGACAGAGATCAAGGAGCACATCTACAGATACCGTCACTATATCGGGGTT |
| | ramosa species | GCGGCTGCTGTCGCTTTGGTGTGCCAACAAGTGTATTACAGGATCTTTCGT |
| | of origin; | ATCCCTAAGAACCTGAGGCACATACCTGCCATTCCGTACGGTCAACAACTA |
| | nucleic acid | AAGGCATTGAGGTCCGATGAGGCTCTTACCAGCAGGACGAAGCGTTTAGTA |
| | sequence; | TTCCCTTTGCTATCTAAGTGTAATGGGGTATACCTTAATAGAATGCCATTT |
| | 14HC_P450_5 | AAGTGGACAATTTATGTCGCGGATCCAGAAATCGCGAGGGCGATTCTTTTT |
| | | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | TTAGACGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 114 | Mucor | MTLIASYNQTLLERVISVVRKNKTSYIGMAILCVVLQQVYASFTVPPKYLRRY |
| | circinelloides | PTVSFFEMMKSFYRKESVLNRNKRLVTPLTNAGHGFYVCRIPLDWTIYVTD |
| | species of | PIAAKTLLLKTDNFPKSHAIFGALGESSPAVKFMGSENVAVSNGEMWKKQR |
| | origin; amino | KIMNPAFHRSQPVKLFGGVMPDLFALIDQDPEHVFVAPRMKSFALDALGLS |
| | acid sequence; | AFGFDFQSLKGDPEGWTSKYNTVISTLFNPFINLFAKYDFLIKYISPERRRVIK |
| | 14HC_P450_6 | ATDEFNVMLSNLADKRRQEILDGEKKDMAENEKDLLTLMIEADIREGVETT |
| | | TTELRHNMAIFFLAGHDTTANTLALCLYQLAKHKHVQKKARQEVLDILGDD |
| | | PCDVTPTLEDLKKLNYVNMVIKENLRRNSPVDNLMARDTHQDIDLNGTFIP |
| | | KGSKVTVNVASIHLNPKIWHDPESFIPERFEPGGEYDGHDGFTWIPFSNGSR |
| | | QCLGLNFSLTEQRVVLCMLLKRYEIEIPKDSIHYNEIVFDKPFTFAPQSLELSF |
| | | KKRY* |
| 115 | Mucor | ATGACCCTGATAGCTTCTTATAATCAGACACTGCTTGAACGTGTTATAAGC |
| | circinelloides | GTGGTTAGGAAGAACAAGACCTCCTATATCGGAATGGCGATTTTATGCGTG |
| | species of | GTTCTGCAGCAAGTGTATGCGTCTTTTACGGTGCCGCCTAAATATTTAAGG |
| | origin; nucleic | AGATACCCGACAGTTAGCTTTTTTGAAATGATGAAGAGCTTTTACCGTAAA |
| | acid sequence; | GAGTCAGTGCTGAACCGTAATAAGCGTCTGGTAACCCCCCTTACTAACGCC |
| | 14HC_P450_6 | GGGCATGGTTTCTATGTGTGTAGAATCCCATTGGATTGGACCATTTATGTT |
| | | ACCGATCCTATAGCAGCTAAGACCCTACTGCTTAAAACAGACAACTTTCCT |
| | | AAATCTCACGCAATATTTGGCGCGTTAGGCGAATCCTCCCCTGCAGTCAAG |
| | | TTCATGGGCTCTGAAAATGTAGCTGTATCTAACGGCGAAATGTGGAAAAAG |
| | | CAAAGAAAAATAATGAACCCCGCGTTTCACAGATCACAGCCCGTTAAGCTT |
| | | TTCGGTGGCGTTATGCCGGATCTATTCGCTCTGATTGACCAGGACCCTGAG |
| | | CATGTCTTTGTTGCGCCGCGTATGAAAAGTTTTGCGCTTGATGCCCTTGGG |
| | | CTGTCAGCCTTCGGTTTTGATTTTCAAAGTCTGAAGGGAGACCCTGAGGGT |
| | | TGGACCAGCAAGTACAATACCGTGATATCTACGCTATTTAACCCTTTTATC |
| | | AACCTTTTCGCTAAGTATGACTTCCTTATTAAGTACATTTCTCCGGAAAGG |
| | | CGTCGTGTGATTAAAGCTACGGACGAGTTTAATGTAATGCTGTCCAATCTA |
| | | GCCGACAAGAGAAGGCAGGAAATCTTGGACGGGGAAAAGAAAGACATGGC |
| | | CGAAAATGAAAAGGACCTATTGACATTAATGATTGAAGCGGATATAAGAG |
| | | AGGGAGTTGAAACAACTACAACGGAGCTGAGACACAATATGGCTATATTTT |
| | | TTCTTGCTGGGCACGACACTACCGCCAATACCCTGGCATTATGTTTATATC |
| | | AACTTGCCAAACATAAGCATGTACAAAAAAAAGCTAGACAAGAGGTGTTG |
| | | GACATCCTAGGAGACGACCCATGCGACGTTACCCCGACTCTAGAGGACTTG |
| | | AAGAAGCTTAACTACGTGAACATGGTGATAAAGGAAAACCTGCGTCGTAAT |
| | | TCCCCCGTGGACAATTTGATGGCCAGGGACACACACCAAGACATAGACCTG |
| | | AATGGTACTTTTATCCCGAAAGGAAGCAAAGTCACTGTTAATGTAGCGAGT |
| | | ATCCATCTGAATCCCAAAATTTGGCATGATCCCGAAAGTTTCATACCGGAG |
| | | AGGTTCGAGCCAGGCGGGGAATATGATGGTCATGATGGATTCACCTGGATT |
| | | CCGTTTAGTAATGGGAGTCGTCAGTGCCTGGGGCTGAATTTTAGTTTAACA |
| | | GAGCAGAGAGTAGTCTTATGTATGTTGCTAAAGAGATACGAAATCGAGAT |
| | | ACCAAAAGATTCAATACATTACAATGAGATTGTCTTCGATAAGCCCTTCAC |
| | | GTTCGCGCCCCAGTCCTTGGAATTAAGCTTTAAAAAGCGTTATTAG |
| 116 | Rhizopus | MDHLIQVYNSSTQVLIPVLQKRSKASYITAAIALIIAQRLYSYFRVPKHLRGFP |
| | microsporus | KLPYFGIAKSFFAKESPRERVKKYILPIINERDGFYISNIPFGWMLYVTNPIAA |
| | species of | KQILLKSNGFPKNHGLLEDMGENLFIEFIGKDNVVLTNGDTWKRQRKVMNP |
| | origin; amino | AFHHSLPIKTMSNVVFSLISVIDQANGTVPVASTMQNFTLDTLGLAIFGFDFK |
| | acid sequence; | ALQGDGDEWTKTYRLVSDCLFDPIINVFSSYSFIFDRIYPRRRRGAMATRKLG |
| | 14HC_P450_7 | EKFLEIAQQKRMEIKSGAFADVPDNEKDLLTLMLEAEEKGDVWTSEDELRH |
| | | NIAVLFLAGHDTTAHALSFCFYHLAKNKDIQQKLRKEVLDLLGDEPVDVVPT |
| | | VEQLKDMQYLNMVIKENLRMNSPADMLFSRDVQEDIVLANTFIPKGTVISIN |
| | | IEALHCNPKLWHNPDQFDPERFAPGGEHEQHEGMTWLPFSNGTRQCLGM |
| | | NFSLFEQRLVIAMILKKYEISIPEDSIHRNHIINDMPFNVAPKSLELTFTKRY* |
| 117 | Rhizopus | ATGGATCATTTAATTCAAGTATACAATAGTTCTACACAAGTATTGATTCCA |
| | microsporus | GTTCTTCAAAAGAGATCAAAGGCATCTTACATTACAGCCGCGATTGCCTTA |
| | species of | ATAATCGCTCAACGACTGTACTCTTACTTTCGTGTACCCAAGCATCTTCGT |
| | origin; nucleic | GGCTTCCCCAAATTACCTTACTTTGGAATAGCAAAGTCATTCTTTGCAAAA |
| | acid sequence; | GAATCACCAAGGGAAAGAGTGAAGAAATATATATTACCTATTATTAATGA |
| | 14HC_P450_7 | ACGCGATGGATTTTATATTAGTAATATCCCTTTTGGATGGATGCTCTACGT |
| | | CACTAACCCTATAGCAGCCAAGCAGATTCTACTCAAATCAAATGGGTTTCC |
| | | CAAGAATCATGGACTACTAGAAGATATGGGTGAAAATCTATTTATAGAAT |
| | | TCATTGGCAAAGATAATGTAGTTCTTACTAATGGAGATACCTGGAAGAGGC |
| | | AAAGAAAGGTCATGAACCCCGCTTTCCATCACTCTTTGCCTATCAAAACCA |
| | | TGTCTAACGTTGTGTTCTCATTGATATCAGTCATCGACCAAGCAAACGGTA |
| | | CAGTTCCGGTTGCATCTACCATGCAGAATTTTACTCTTGATACACTTGGTC |
| | | TTGCTATCTTTGGCTTTGACTTTAAAGCATTACAGGGCGATGGGGATGAAT |
| | | GGACCAAGACTTACAGACTTGTAAGCGACTGTCTCTTTGACCCTATTATCA |
| | | ATGTCTTCAGCTCGTACTCTTTTATCTTTGACCGCATCTATCCTAGACGACG |
| | | AAGAGGCGCTATGGCTACTAGAAAACTTGGTGAAAAATTCTTGGAGATTGC |
| | | GCAACAAAAGCGCATGGAAATCAAAAGTGGTGCTTTTGCAGATGTACCAGA |
| | | CAATGAAAAGGATCTTTTGACACTTATGCTTGAGGCTGAAGAAAAGGGCGA |
| | | TGTATGGACATCAGAAGACGAGCTTAGACATAATATTGCAGTCTTGTTTTT |
| | | AGCTGGACATGATACCACTGCTCATGCTCTGTCCTTCTGTTTCTACCATCTT |
| | | GCAAAGAATAAGGATATTCAACAAAAGCTAAGAAAAGAAGTGCTTGATCT |
| | | TCTGGGCGATGAGCCAGTTGATGTAGTGCCTACAGTTGAGCAACTCAAAGA |
| | | TATGCAATACCTCAATATGGTCATAAAAGAGAATCTGCGTATGAATAGCCC |
| | | AGCTGATATGCTATTTTCACGAGATGTACAAGAAGACATCGTTTTGGCCAA |
| | | CACGTTCATTCCCAAGGGTACTGTTATTTCAATCAATATAGAGGCTTTGCA |
| | | CTGCAACCCCAAGCTTTGGCATAACCCAGACCAATTTGATCCTGAAAGATT |
| | | TGCTCCTGGTGGTGAACATGAACAACATGAAGGTATGACTTGGCTACCTTT |
| | | TAGTAATGGAACCCGTCAATGCCTTGGCATGAATTTCTCTCTGTTTGAGCA |
| | | ACGATTGGTTATTGCTATGATCTTGAAAAAGTATGAGATTAGTATTCCGGA |
| | | AGACTCCATTCATCGTAATCATATTATTAATGACATGCCATTCAATGTCGC |
| | | GCCCAAGTCACTGGAACTGACTTTTACCAAGCGTTATTAA |
| 118 | Rhizopus | MDHLIQVYNSSTQVLIPVLQKRSKASYITAAIALIIAQRLYSYFRVPKHLRGFP |
| | microsporus | KLPYFGIAKSLLTRESPRERVKKYVLPIIDENDGFYISKIPFGWMLYIANPVAA |
| | species of | KQLLLKSSGFPKNHGLLDNMGENLFVEFIGKDNVALSNGDTWKRQRKVMN |
| | origin; amino | PAFHHSLPIKTMSKVVFSLISAIEQANGTIPVASAMQNFTLDTLGLAIFGFDF |
| | acid sequence; | KALQGDPDEWTKTYRFVSDCIFDPVINVFSSYSFIIDRIYPRRRRGAIATKKLS |
| | 14HC_P450_8 | EKFLKIAQQKRKEIKSGAYADVPDNEKDLLTLMLEAEEKGDTWTSEDELRH |
| | | NIAILFVAGHDTTAHALSFCFYHLAKNKDVQQKLRKEVLSILGDKPVDVVPT |
| | | VEQLKNMQYLNMVIKENLRINSPADMLFSRDVKEDTILANTLIPKGTVVTIN |
| | | IEALHYNPRLWHNPDQFDPERFAPGGEHEKHEGMTWLPFSNGTRQCLGM |
| | | NFSLFEQRLVIAMILKKYEISIPEDSIHRDHIVSDIPFNGAPKSLKLTFTKRH* |
| 119 | Rhizopus | ATGGATCATCTTATTCAGGTGTACAACAGCTCAACGCAAGTTTTAATTCCG |
| | microsporus | GTTCTTCAGAAAAGGTCTAAGGCGAGTTACATTACCGCCGCAATAGCGCTG |
| | species of | ATTATAGCACAGAGGCTATATTCTTACTTTCGTGTACCTAAACATCTGCGT |
| | origin; nucleic | GGCTTTCCCAAATTACCGTACTTCGGCATCGCCAAATCACTACTGACTAGG |
| | acid sequence; | GAAAGCCCAAGAGAGAGAGTCAAGAAGTACGTATTACCTATAATCGACGAG |
| | 14HC_P450_8 | AACGATGGTTTCTACATAAGTAAAATTCCCTTTGGGTGGATGCTGTATATC |
| | | GCAAATCCGGTGGCTGCGAAACAACTTTTGCTGAAGTCTTCAGGGTTCCCC |
| | | AAGAACCACGGCTTATTAGATAACATGGGCGAAAATTTATTCGTGGAGTTT |
| | | ATTGGAAAGGACAACGTCGCTCTTAGCAATGGGGATACGTGGAAGAGGCAG |
| | | AGAAAAGTCATGAACCCAGCCTTCCACCATTCTTTGCCTATTAAGACCATG |
| | | TCTAAAGTAGTGTTCTCACTGATCTCAGCGATTGAACAGGCAAATGGCACA |
| | | ATCCCCGTCGCGTCAGCTATGCAGAATTTTACGCTAGACACTTTGGGGTTG |
| | | GCTATTTTCGGGTTTGACTTCAAAGCCTTACAAGGAGATCCCGACGAATGG |
| | | ACAAAAACATACCGTTTTGTCTCTGACTGTATTTTTGACCCCGTTATCAAC |
| | | GTCTTTTCTTCCTACAGTTTCATAATCGACCGTATATATCCTAGAAGAAGG |
| | | CGTGGCGCAATTGCAACGAAAAAGTTAAGTGAGAAATTCCTTAAAATCGCC |
| | | CAACAAAAAAGGAAGGAGATAAAATCTGGAGCCTACGCCGACGTGCCTGAT |
| | | AACGAGAAAGATTTGCTAACGCTGATGTTGGAAGCAGAGGAAAAGGGCGA |
| | | CACTTGGACCAGCGAAGATGAGCTTAGACACAATATCGCGATCCTTTTCGT |
| | | CGCGGGCCATGATACAACGGCCCACGCTCTATCATTCTGTTTTTATCATCTG |
| | | GCCAAGAACAAAGACGTACAACAGAAGCTGAGGAAGGAGGTTCTAAGTATC |
| | | CTAGGGGATAAGCCCGTGGATGTTGTCCCCACCGTGGAGCAATTAAAGAAC |
| | | ATGCAGTACCTGAATATGGTGATAAAAGAAAATCTTAGGATTAACTCACCC |
| | | GCCGATATGTTATTCAGCAGGGATGTCAAGGAGGACACGATATTGGCTAAC |
| | | ACTCTAATCCCGAAAGGGACAGTGGTCACGATCAATATCGAAGCCCTACAT |
| | | TACAATCCGAGGTTATGGCATAACCCGGATCAGTTCGATCCGGAAAGGTTC |
| | | GCTCCTGGAGGCGAACATGAGAAACACGAAGGCATGACATGGTTACCATTC |
| | | TCCAATGGAACACGTCAGTGTTTAGGAATGAATTTTAGCTTGTTCGAGCAA |
| | | AGATTAGTTATCGCCATGATTTTGAAGAAGTACGAGATATCCATACCCGAG |
| | | GATTCTATTCATAGGGACCATATAGTGAGTGATATACCCTTTAACGGTGCT |
| | | CCTAAATCTCTGAAGCTAACTTTTACTAAGAGGCACTAG |
| 120 | Homo sapiens | MDLIPNLAVETWLLLAVSLVLLYLYGTRTHGLFKRLGIPGPTPLPLLGNVLSY |
| | species of | RQGLWKFDTECYKKYGKMWGTYEGQLPVLAITDPDVIRTVLVKECYSVFTN |
| | origin; amino | RRSLGPVGFMKSAISLAEDEEWKRIRSLLSPTFTSGKLKEMFPIIAQYGDVLV |
| | acid sequence; | RNLRREAEKGKPVTLKDIFGAYSMDVITGTSFGVNIDSLNNPQDPFVESTKK |
| | 14HC_P450_9 | FLKFGFLDPLFLSIILFPFLTPVFEALNVSLFPKDTINFLSKSVNRMKKSRLND |
| | | KQKHRLDFLQLMIDSQNSKETESHKALSDLELAAQSIIFIFAGYETTSSVLSFT |
| | | LYELATHPDVQQKLQKEIDAVLPNKAPPTYDAVVQMEYLDMVVNETLRLFP |
| | | VAIRLERTCKKDVEINGVFIPKGSMVVIPTYALHHDPKYWTEPEEFRPERFS |
| | | KKKDSIDPYIYTPFGTGPRNCIGMRFALMNMKLALIRVLQNFSFKPCKETQIP |
| | | LKLDTQGLLQPEKPIVLKVDSRDGTLSGE* |
| 121 | Homo sapiens | ATGGACCTTATCCCAAACCTGGCGGTCGAGACTTGGTTACTTCTGGCCGTA |
| | species of | AGCCTAGTTTTGCTGTATTTGTATGGTACTCGTACTCACGGTTTATTCAAG |
| | origin; nucleic | AGACTAGGTATTCCAGGGCCTACTCCCTTACCTCTGTTAGGAAATGTTCTG |
| | acid sequence; | TCTTACAGACAAGGCCTATGGAAATTCGATACGGAGTGCTACAAGAAATAT |
| | 14HC_P450_9 | GGCAAAATGTGGGGCACCTACGAGGGACAGCTTCCCGTGCTGGCGATTACG |
| | | GATCCTGACGTAATAAGAACCGTGTTGGTAAAAGAATGCTACAGTGTATTC |
| | | ACCAACCGTAGGTCCTTAGGACCTGTCGGATTCATGAAATCCGCGATAAGC |
| | | CTAGCAGAGGACGAAGAATGGAAGAGGATCAGATCCTTATTAAGCCCAACG |
| | | TTTACATCAGGAAAGTTGAAAGAAATGTTCCCCATTATTGCTCAGTACGGT |
| | | GATGTCCTGGTCAGGAATTTGAGGCGTGAAGCTGAGAAGGGTAAACCCGTT |
| | | ACACTTAAAGACATCTTTGGAGCTTACTCCATGGACGTCATCACCGGCACA |
| | | TCTTTCGGTGTCAACATCGATAGTCTTAACAATCCTCAAGATCCCTTTGTG |
| | | GAGAGCACAAAGAAGTTTCTAAAATTTGGGTTTTTGGACCCTCTATTTCTA |
| | | TCTATAATACTTTTCCCGTTTCTTACGCCTGTCTTCGAGGCTTTAAATGTCT |
| | | CACTTTTTCCGAAGGATACCATTAATTTCTTGAGTAAATCAGTCAATCGTA |
| | | TGAAAAAATCACGTTTGAATGACAAGCAGAAACATCGTCTGGACTTTTTAC |
| | | AACTGATGATCGATTCTCAGAATTCCAAGGAAACTGAGTCTCACAAGGCGC |
| | | TTTCAGATTTGGAGCTAGCTGCTCAGAGTATAATTTTTATCTTTGCCGGGT |
| | | ATGAGACGACTAGCTCTGTCCTGTCATTTACTCTTTACGAGTTAGCAACGC |
| | | ATCCTGATGTTCAACAAAAACTTCAGAAAGAAATTGATGCTGTACTACCGA |
| | | ACAAAGCGCCGCCCACTTACGATGCAGTCGTGCAAATGGAATATCTTGATA |
| | | TGGTGGTAAACGAGACACTGAGGCTATTCCCAGTAGCTATTAGGTTGGAGC |
| | | GTACTTGTAAAAAGGATGTCGAGATTAACGGAGTTTTTATACCCAAGGGCT |
| | | CAATGGTGGTTATTCCGACGTATGCACTTCACCACGATCCTAAATATTGGA |
| | | CAGAACCAGAAGAATTCAGACCGGAGAGGTTCTCAAAAAAGAAAGACTCAA |
| | | TCGACCCATACATATATACACCCTTTGGCACGGGTCCGAGGAATTGCATTG |
| | | GTATGAGGTTTGCTTTGATGAATATGAAATTAGCTTTAATAAGAGTACTGC |
| | | AAAATTTTTCCTTCAAACCCTGCAAGGAAACCCAGATACCACTTAAACTGG |
| | | ACACTCAGGGTCTGCTGCAGCCCGAAAAGCCAATAGTGTTGAAGGTAGATT |
| | | CTCGTGACGGCACCTTGAGTGGAGAATGA |
| 122 | Homo sapiens | MALSQSVPFSATELLLASAIFCLVFWVLKGLRPRVPKGLKSPPEPWGWPLLG |
| | species of | HVLTLGKNPHLALSRMSQRYGDVLQIRIGSTPVLVLSRLDTIRQALVRQGDD |
| | origin; amino | FKGRPDLYTSTLITDGQSLTFSTDSGPVWAARRRLAQNALNTFSIASDPASSS |
| | acid sequence; | SCYLEEHVSKEAKALISRLQELMAGPGHFDPYNQVVVSVANVIGAMCFGQHF |
| | 14HC_P450_10 | PESSDEMLSLVKNTHEFVETASSGNPLDFFPILRYLPNPALQRFKAFNQRFL |
| | | WFLQKTVQEHYQDFDKNSVRDITGALFKHSKKGPRASGNLIPQEKIVNLVN |
| | | DIFGAGFDTVTTAISWSLMYLVTKPEIQRKIQKELDTVIGRERRPRLSDRPQL |
| | | PYLEAFILETFRHSSFLPFTIPHSTTRDTTLNGFYIPKKCCVFVNQWQVNHDP |
| | | ELWEDPSEFRPERFLTADGTAINKPLSEKMMLFGMGKRRCIGEVLAKWEIF |
| | | LFLAILLQQLEFSVPPGVKVDLTPIYGLTMKHARCEHVQARLRFSIN* |
| 123 | Homo sapiens | ATGGCCTTGTCTCAATCTGTTCCATTTTCTGCTACTGAGTTGTTGTTGGCT |
| | species of | TCTGCTATTTTCTGTTTGGTTTTCTGGGTCTTGAAGGGTTTAAGACCAAGA |
| | origin; nucleic | GTTCCAAAAGGTTTGAAATCTCCACCAGAACCATGGGGTTGGCCATTATTG |
| | acid sequence; | GGTCATGTTTTGACTTTGGGTAAGAACCCACATTTGGCTTTGTCTAGAATG |
| | 14HC_P450_10 | TCTCAAAGATACGGTGATGTCTTGCAAATCAGAATTGGTTCTACTCCAGTT |
| | | TTGGTCTTGTCTAGATTGGATACCATTAGACAAGCCTTGGTTAGACAAGGT |
| | | GATGATTTTAAAGGTAGGCCAGACTTGTACACCTCTACTTTGATTACTGAT |
| | | GGTCAGTCTTTGACCTTCTCTACTGATTCTGGTCCAGTTTGGGCTGCTAGA |
| | | CGTAGATTGGCTCAAAATGCTTTGAATACTTTCTCGATTGCTTCCGATCCA |
| | | GCTTCTTCTTCATCTTGTTATTTGGAGGAACACGTGTCCAAAGAAGCTAAG |
| | | GCTTTGATTTCTAGGTTGCAAGAATTGATGGCTGGTCCAGGTCATTTTGAT |
| | | CCATACAATCAAGTTGTTGTCTCCGTTGCTAATGTTATTGGTGCTATGTGT |
| | | TTCGGTCAACACTTTCCAGAATCTTCAGACGAAATGTTGTCCTTGGTTAAG |
| | | AACACCCACGAATTTGTTGAAACTGCTTCTTCTGGTAACCCTTTGGATTTC |
| | | TTTCCAATCTTGCGTTACTTGCCAAATCCAGCATTGCAAAGATTCAAGGCT |
| | | TTTAACCAGAGATTCTTGTGGTTCTTGCAAAAGACCGTTCAAGAACACTAC |
| | | CAAGATTTCGATAAGAACTCCGTTAGAGATATTACTGGCGCTTTGTTTAAG |
| | | CACTCTAAGAAAGGTCCAAGAGCCTCTGGTAATTTGATTCCACAAGAAAAG |
| | | ATCGTCAACCTGGTTAACGATATCTTTGGTGCTGGTTTCGATACTGTTACT |
| | | ACTGCTATTTCTTGGTCCTTGATGTACTTGGTTACCAAGCCAGAAATTCAG |
| | | AGGAAGATCCAGAAAGAATTGGACACCGTTATTGGTAGAGAAAGAAGGCC |
| | | AAGATTGTCTGATAGACCACAATTGCCATATTTGGAAGCCTTCATTTTGGA |
| | | AACTTTCAGGCACTCTTCTTTCTTGCCATTCACTATTCCACATTCTACTACT |
| | | AGAGATACCACTTTGAACGGTTTCTACATCCCAAAGAAATGCTGCGTTTTC |
| | | GTCAATCAATGGCAAGTTAATCACGATCCAGAATTGTGGGAAGATCCTTCT |
| | | GAGTTTAGACCAGAAAGATTCTTGACTGCTGATGGTACAGCTATTAACAAA |
| | | CCATTGTCCGAGAAGATGATGTTGTTTGGTATGGGTAAGAGAAGATGCATC |
| | | GGTGAAGTTTTGGCTAAGTGGGAAATTTTCTTGTTCTTGGCCATTTTGTTG |
| | | CAGCAGTTGGAATTTTCAGTTCCACCAGGTGTTAAGGTTGATTTGACTCCA |
| | | ATCTATGGTTTGACTATGAAGCACGCTAGATGTGAACACGTTCAAGCTAGA |
| | | TTGAGATTCTCCATCAACTAA |
| 124 | Homo sapiens | MLASGMLLVALLVCLTVMVLMSVWQQRKSKGKLPPGPTPLPFIGNYLQLNT |
| | species of | EQMYNSLMKISERYGPVFTIHLGPRRVVVLCGHDAVREALVDQAEEFSGRGE |
| | origin; amino | QATFDWVFKGYGVVFSNGERAKQLRRFSIATLRDFGVGKRGIEERIQEEAGF |
| | acid sequence; | LIDALRGTGGANIDPTFFLSRTVSNVISSIVFGDRFDYKDKEFLSLLRMMLGIF |
| | 14HC_P450_11 | QFTSTSTGQLYEMFSSVMKHLPGPQQQAFQLLQGLEDFIAKKVEHNQRTLD |
| | | PNSPRDFIDSFLIRMQEEEKNPNTEFYLKNLVMTTLNLFIGGTETVSTTLRYG |
| | | FLLLMKHPEVEAKVHEEIDRVIGKNRQPKFEDRAKMPYMEAVIHEIQRFGD |
| | | VIPMSLARRVKKDTKFRDFFLPKGTEVYPMLGSVLRDPSFFSNPQDFNPQHF |
| | | LNEKGQFKKSDAFVPFSIGKRNCFGEGLARMELFLFFTTVMQNFRLKSSQSP |
| | | KDIDVSPKHVGFATIPRNYTMSFLPR* |
| 125 | Homo sapiens | ATGTTGGCTTCTGGTATGTTGTTGGTTGCTTTGTTGGTTTGTTTGACCGTT |
| | species of | ATGGTTTTGATGTCCGTTTGGCAACAAAGAAAATCTAAAGGCAAATTGCCA |
| | origin; nucleic | CCAGGTCCAACTCCATTGCCTTTTATTGGTAATTACTTGCAGTTGAACACC |
| | acid sequence; | GAGCAGATGTACAATTCCTTGATGAAGATCTCTGAAAGATACGGTCCAGTT |
| | 14HC_P450_11 | TTCACCATTCATTTGGGTCCTAGAAGAGTTGTTGTTTTGTGTGGTCATGAT |
| | | GCTGTTAGAGAAGCTTTGGTTGATCAAGCCGAAGAATTTTCTGGTAGAGGT |
| | | GAACAAGCTACTTTCGACTGGGTTTTCAAAGGTTATGGTGTCGTTTTCTCT |
| | | AACGGTGAAAGAGCTAAACAGTTGAGAAGATTCTCTATTGCCACCTTGAGA |
| | | GATTTTGGTGTTGGTAAGAGAGGTATCGAAGAAAGAATCCAAGAAGAAGC |
| | | CGGTTTCTTGATTGATGCTTTGAGAGGTACTGGTGGTGCTAATATTGATCC |
| | | AACATTCTTCTTGTCCAGGACCGTTTCTAACGTTATTTCCTCTATTGTGTTC |
| | | GGTGACAGATTCGACTACAAAGACAAAGAGTTCCTGTCCTTGTTGAGAATG |
| | | ATGTTGGGTATTTTCCAATTCACCTCCACTTCCACTGGTCAATTATACGAA |
| | | ATGTTCTCCTCAGTCATGAAGCACTTGCCAGGTCCACAACAACAAGCTTTT |
| | | CAATTATTGCAAGGTTTGGAGGACTTCATTGCCAAGAAGGTTGAACATAAT |
| | | CAGAGAACTTTGGACCCAAACTCTCCAAGAGATTTCATCGATTCATTCTTG |
| | | ATCAGGATGCAAGAGGAAGAAAAGAACCCAAACACCGAGTTTTACTTGAAG |
| | | AACTTGGTTATGACCACCTTGAACTTGTTCATTGGTGGTACTGAAACTGTT |
| | | TCCACTACTTTGAGATATGGCTTCCTGTTGTTGATGAAGCACCCAGAAGTT |
| | | GAAGCTAAGGTTCATGAAGAAATCGATAGAGTCATCGGTAAGAACAGACA |
| | | ACCTAAGTTTGAAGATAGAGCCAAGATGCCTTATATGGAAGCTGTTATTCA |
| | | CGAAATCCAAAGATTCGGTGATGTCATCCCAATGTCTTTGGCAAGAAGAGT |
| | | TAAGAAGGATACCAAGTTCAGGGACTTCTTCTTACCAAAAGGTACTGAAGT |
| | | TTACCCAATGTTGGGTTCCGTTTTGAGAGATCCATCTTTCTTTAGCAACCC |
| | | ACAAGACTTTAACCCACAGCATTTCTTGAACGAAAAGGGCCAGTTTAAGAA |
| | | ATCCGATGCTTTTGTTCCATTCTCCATTGGCAAGAGAAATTGCTTTGGTGA |
| | | AGGTTTGGCTAGGATGGAATTATTCTTGTTCTTCACTACCGTCATGCAGAA |
| | | CTTCAGATTGAAGTCATCTCAATCCCCAAAGGATATCGATGTTTCCCCAAA |
| | | ACATGTTGGTTTCGCTACTATTCCAAGAAACTACACCATGTCTTTCTTGCCC |
| | | AGATAA |
| 126 | Homo sapiens | MLASGLLLVTLLACLTVMVLMSVWRQRKSRGKLPPGPTPLPFIGNYLQLNT |
| | species of | EQMYNSLMKISERYGPVFTIHLGPRRVVVLCGHDAVKEALVDQAEEFSGRGE |
| | origin; amino | QATFDWLFKGYGVAFSNGERAKQLRRFSIATLRGFGVGKRGIEERIQEEAGF |
| | acid sequence; | LIDALRGTHGANIDPTFFLSRTVSNVISSIVFGDRFDYEDKEFLSLLRMMLGS |
| | 14HC_P450_12 | FQFTATSTGQLYEMFSSVMKHLPGPQQQAFKELQGLEDFIAKKVEHNQRTL |
| | | DPNSPRDFIDSFLIRMQEEEKNPNTEFYLKNLVMTTLNLFFAGTETVSTTLR |
| | | YGFLLLMKHPEVEAKVHEEIDRVIGKNRQPKFEDRAKMPYTEAVIHEIQRFG |
| | | DMLPMGLAHRVNKDTKFRDFFLPKGTEVFPMLGSVLRDPRFFSNPRDFNP |
| | | QHFLDKKGQFKKSDAFVPFSIGKRYCFGEGLARMELFLFFTTIMQNFRFKSP |
| | | QSPKDIDVSPKHVGFATIPRNYTMSFLPR* |
| 127 | Homo sapiens | ATGTTGGCTTCTGGTTTGTTGTTGGTTACTTTGTTGGCTTGTTTGACCGTT |
| | species of | ATGGTCTTGATGTCTGTTTGGAGACAAAGAAAGTCTAGAGGTAAATTGCCA |
| | origin; nucleic | CCAGGTCCAACTCCATTGCCTTTTATTGGTAATTACTTGCAGTTGAACACC |
| | acid sequence; | GAGCAGATGTACAATTCCTTGATGAAGATCTCTGAAAGATACGGTCCAGTT |
| | 14HC_P450_12 | TTCACCATTCATTTGGGTCCTAGAAGAGTTGTTGTTTTGTGTGGTCATGAT |
| | | GCTGTCAAAGAAGCTTTGGTTGATCAAGCCGAAGAATTTTCTGGTAGAGGT |
| | | GAACAAGCTACTTTCGACTGGTTGTTTAAAGGTTATGGTGTTGCTTTCTCC |
| | | AACGGTGAAAGAGCTAAACAATTGAGAAGATTCTCCATAGCTACCTTGAGA |
| | | GGTTTCGGTGTTGGTAAAAGAGGTATCGAAGAAAGAATCCAAGAAGAAGC |
| | | CGGTTTCTTGATTGATGCTTTGAGAGGTACTCATGGTGCTAATATTGATCC |
| | | AACGTTCTTCTTGTCTAGGACCGTTTCTAACGTTATCTCCTCTATTGTCTTT |
| | | GGTGACAGATTTGACTACGAGGACAAAGAGTTCTTGTCCTTGTTGAGAATG |
| | | ATGTTGGGTTCTTTCCAATTCACTGCTACTTCCACTGGTCAACTGTACGAA |
| | | ATGTTTTCCTCAGTTATGAAGCACTTGCCAGGTCCACAACAACAAGCTTTT |
| | | AAAGAATTGCAAGGTTTGGAGGACTTCATTGCCAAGAAGGTTGAACATAAT |
| | | CAGAGAACTTTGGACCCAAACTCTCCAAGAGATTTCATCGATTCATTCTTG |
| | | ATCAGGATGCAAGAGGAAGAAAAGAACCCTAATACTGAGTTCTACCTGAAG |
| | | AACTTGGTTATGACTACCCTGAATTTGTTCTTCGCTGGTACTGAAACTGTT |
| | | TCTACCACTTTGAGATACGGATTCTTGTTGTTAATGAAGCACCCAGAAGTT |
| | | GAAGCCAAGGTTCATGAAGAAATCGATAGAGTTATCGGTAAGAACAGACA |
| | | GCCAAAGTTTGAAGATAGAGCTAAGATGCCATACACCGAAGCTGTTATTCA |
| | | CGAAATTCAAAGATTCGGTGACATGTTGCCAATGGGTTTAGCTCATAGAGT |
| | | TAACAAGGATACCAAGTTCAGGGATTTCTTCTTGCCTAAAGGTACTGAAGT |
| | | TTTCCCAATGTTGGGTAGCGTTTTGAGAGATCCAAGATTCTTTTCAAACCC |
| | | AAGAGACTTTAACCCTCAGCATTTCTTGGATAAGAAGGGCCAATTCAAGAA |
| | | ATCCGATGCTTTTGTTCCATTCTCCATTGGTAAGAGATACTGTTTTGGTGA |
| | | AGGTTTGGCTAGGATGGAACTGTTCTTGTTCTTCACTACCATCATGCAGAA |
| | | CTTCAGATTCAAGTCTCCACAATCTCCAAAGGATATCGATGTTTCCCCAAA |
| | | ACATGTTGGTTTCGCTACTATTCCAAGAAACTACACCATGTCTTTCTTGCC |
| | | AAGATAA |
| 128 | Homo sapiens | MDSLVVLVLCLSCLLLLSLWRQSSGRGKLPPGPTPLPVIGNILQIGIKDISKSLT |
| | species of | NLSKVYGPVFTLYFGLKPIVVLHGYEAVKEALIDLGEEFSGRGIFPLAERANR |
| | origin; amino | GFGIVFSNGKKWKEIRRFSLMTLRNFGMGKRSIEDRVQEEARCLVEELRKTK |
| | acid sequence; | ASPCDPTFILGCAPCNVICSIIFHKRFDYKDQQFLNLMEKLNENIKILSSPWIQ |
| | 14HC_P450_13 | ICNNFSPIIDYFPGTHNKLLKNVAFMKSYILEKVKEHQESMDMNNPQDFIDC |
| | | FLMKMEKEKHNQPSEFTIESLENTAVDLFGAGTETTSTTLRYALLLLLKHPE |
| | | VTAKVQEEIERVIGRNRSPCMQDRSHMPYTDAVVHEVQRYIDLLPTSLPHA |
| | | VTCDIKFRNYLIPKGTTILISLTSVLHDNKEFPNPEMFDPHHFLDEGGNFKKS |
| | | KYFMPFSAGKRICVGEALAGMELFLFLTSILQNFNLKSLVDPKNLDTTPVVN |
| | | GFASVPPFYQLCFIPV* |
| 129 | Homo sapiens | ATGGATTCCTTGGTTGTTTTGGTCTTGTGTTTGTCTTGCCTGTTGTTGTTG |
| | species of | TCTCTTTGGAGACAATCTTCTGGTAGAGGTAAATTGCCACCAGGTCCAACT |
| | origin; nucleic | CCATTGCCAGTTATTGGTAATATCTTGCAGATCGGTATCAAGGACATCTCT |
| | acid sequence; | AAGTCTTTGACCAACTTGTCTAAAGTTTACGGTCCAGTTTTCACCTTGTAC |
| | 14HC_P450_13 | TTTGGTTTGAAACCTATCGTTGTCTTGCATGGTTATGAAGCTGTAAAAGAA |
| | | GCCTTGATCGATTTGGGTGAAGAATTTTCAGGTAGGGGTATTTTCCCATTG |
| | | GCTGAAAGAGCTAATAGAGGTTTTGGTATCGTTTTCTCCAATGGCAAGAAG |
| | | TGGAAAGAGATTAGAAGGTTCTCTTTGATGACCTTGAGAAACTTTGGTATG |
| | | GGCAAGAGATCCATCGAAGATAGAGTTCAAGAAGAAGCTCGTTGCTTGGTA |
| | | GAAGAATTGAGAAAGACAAAAGCTTCTCCATGTGATCCAACCTTCATTTTG |
| | | GGTTGTGCTCCATGTAACGTTATCTGCTCTATTATCTTCCACAAGAGGTTC |
| | | GATTACAAGGACCAACAGTTCTTGAACCTGATGGAAAAGTTGAACGAGAAC |
| | | ATCAAGATTCTGTCCTCTCCATGGATTCAAATCTGCAACAATTTCTCCCCA |
| | | ATCATCGATTACTTTCCAGGTACTCATAACAAGCTGTTGAAGAACGTTGCT |
| | | TTCATGAAGTCCTACATCTTGGAAAAGGTCAAAGAACACCAAGAGTCCATG |
| | | GATATGAACAACCCACAAGATTTCATCGACTGTTTCTTGATGAAGATGGAA |
| | | AAGGAAAAGCACAATCAGCCATCTGAGTTCACCATTGAATCTTTGGAAAAC |
| | | ACCGCTGTTGATTTGTTTGGTGCTGGTACTGAAACTACTTCTACTACTTTG |
| | | AGATACGCCCTGCTGTTGTTATTGAAACATCCAGAAGTTACCGCTAAGGTC |
| | | CAAGAAGAAATTGAAAGAGTTATCGGTAGGAACAGATCTCCATGTATGCA |
| | | AGATAGATCTCATATGCCATACACTGATGCCGTTGTTCACGAAGTTCAAAG |
| | | GTATATCGATTTGTTGCCAACCTCTTTGCCACATGCTGTTACTTGTGATAT |
| | | CAAGTTCCGTAACTACTTGATCCCAAAGGGTACTACCATCTTGATTTCTTT |
| | | GACTTCTGTCCTGCACGACAACAAAGAATTTCCAAATCCAGAAATGTTCGA |
| | | CCCTCATCACTTCTTGGATGAAGGTGGTAATTTCAAGAAATCCAAGTACTT |
| | | CATGCCATTCTCTGCCGGTAAAAGAATTTGTGTTGGTGAAGCTTTGGCTGG |
| | | CATGGAATTATTCTTGTTCTTGACCTCAATCCTGCAAAACTTCAACCTGAA |
| | | GTCTTTGGTTGATCCAAAGAACTTGGATACCACTCCAGTTGTTAATGGTTT |
| | | TGCTTCTGTTCCACCATTCTACCAATTGTGCTTCATTCCAGTTTAA |
| 130 | Homo sapiens | MDPFVVLVLCLSCLLLLSIWRQSSGRGKLPPGPTPLPVIGNILQIDIKDVSKSL |
| | species of | TNLSKIYGPVFTLYFGLERMVVLHGYEVVKEALIDLGEEFSGRGHFPLAERA |
| | origin; amino | NRGFGIVFSNGKRWKEIRRFSLMTLRNFGMGKRSIEDRVQEEARCLVEELRK |
| | acid sequence; | TKASPCDPTFILGCAPCNVICSIIFQKRFDYKDQQFLNLMEKLNENIRIVSTP |
| | 14HC_P450_14 | WIQICNNFPTIIDYFPGTHNKLLKNLAFMESDILEKVKEHQESMDINNPRDFI |
| | | DCFLIKMEKEKQNQQSEFTIENLVITAADLLGAGTETTSTTLRYALLLLLKHP |
| | | EVTAKVQEEIERVVGRNRSPCMQDRGHMPYTDAVVHEVQRYIDLIPTSLPH |
| | | AVTCDVKFRNYLIPKGTTILTSLTSVLHDNKEFPNPEMFDPRHFLDEGGNFK |
| | | KSNYFMPFSAGKRICVGEGLARMELFLFLTFILQNFNLKSLIDPKDLDTTPVV |
| | | NGFASVPPFYQLCFIPV* |
| 131 | Homo sapiens | ATGGACCCATTCGTTGTTTTGGTTTTGTGTTTGTCTTGCCTGCTGTTGTTG |
| | species of | TCTATTTGGAGACAATCTTCTGGTAGAGGTAAATTGCCACCAGGTCCAACT |
| | origin; nucleic | CCATTGCCAGTTATTGGTAATATCTTGCAGATCGACATCAAGGACGTTTCT |
| | acid sequence; | AAGTCTTTGACCAACCTGTCTAAAATCTACGGTCCAGTTTTCACCTTGTAC |
| | 14HC_P450_14 | TTCGGTTTGGAAAGAATGGTTGTCTTGCATGGTTACGAAGTTGTCAAAGAA |
| | | GCCTTGATTGATTTGGGTGAAGAATTTTCAGGTAGAGGTCATTTTCCATTG |
| | | GCTGAAAGAGCTAATAGAGGTTTCGGTATCGTTTTCTCTAATGGCAAGAGA |
| | | TGGAAAGAAATCCGTAGGTTCTCTTTGATGACCTTGAGAAATTTTGGTATG |
| | | GGCAAGCGTTCCATCGAAGATAGAGTTCAAGAAGAAGCTCGTTGCTTGGTA |
| | | GAAGAATTGAGAAAGACAAAAGCTTCTCCATGTGATCCAACCTTCATTTTG |
| | | GGTTGTGCTCCATGTAACGTTATCTGCTCAATTATCTTCCAGAAGAGGTTC |
| | | GATTACAAGGACCAACAGTTCTTGAACCTGATGGAAAAGTTGAACGAAAAC |
| | | ATCAGGATTGTCTCTACCCCATGGATTCAAATCTGTAACAACTTCCCAACC |
| | | ATCATCGATTACTTTCCAGGTACTCATAACAAGCTGTTGAAGAACTTGGCT |
| | | TTCATGGAATCCGATATCTTGGAAAAGGTCAAAGAACACCAAGAGTCCATG |
| | | GATATTAACAACCCAAGAGATTTCATCGACTGCTTTCTGATCAAAATGGAA |
| | | AAGGAAAAGCAGAACCAGCAATCCGAGTTCACTATTGAAAACTTGGTTATT |
| | | ACCGCTGCCGATTTGTTAGGTGCTGGTACTGAAACTACTTCTACTACTTTG |
| | | AGATACGCCCTGCTGCTTTTGTTGAAACATCCAGAAGTTACTGCTAAGGTC |
| | | CAAGAAGAAATCGAAAGAGTTGTCGGTAGAAACAGATCTCCATGTATGCAA |
| | | GATAGAGGTCACATGCCATATACTGATGCAGTTGTTCATGAAGTCCAAAGG |
| | | TACATTGATTTGATCCCAACTTCTTTGCCACATGCTGTTACTTGTGATGTC |
| | | AAGTTTAGGAACTACTTGATCCCTAAGGGTACTACTATTTTGACCTCTTTG |
| | | ACTTCTGTCCTGCACGATAACAAAGAGTTTCCAAATCCAGAAATGTTCGAC |
| | | CCAAGACACTTCTTGGATGAAGGTGGTAATTTCAAGAAATCCAACTACTTC |
| | | ATGCCATTCTCTGCCGGTAAAAGAATTTGTGTTGGTGAAGGTTTGGCCAGG |
| | | ATGGAATTATTCTTGTTCTTGACTTTCATCCTGCAAAACTTCAACCTGAAG |
| | | TCATTGATCGATCCAAAGGATTTGGATACCACTCCAGTTGTTAATGGTTTT |
| | | GCTTCTGTTCCACCATTCTACCAATTGTGCTTTATCCCAGTTTAA |
| 132 | Homo sapiens | MGLEALVPLAVIVAIFLLLVDLMHRRQRWAARYPPGPLPLPGLGNLLHVDF |
| | species of | QNTPYCFDQLRRRFGDVFSLQLAWTPVVVLNGLAAVREALVTHGEDTADR |
| | origin; amino | PPVPITQILGFGPRSQGVFLARYGPAWREQRRFSVSTLRNLGLGKKSLEQWV |
| | acid sequence; | TEEAACLCAAFANHSGRPFRPNGLLDKAVSNVIASLTCGRRFEYDDPRFLRL |
| | 14HC_P450_15 | LDLAQEGLKEESGFLREVLNAVPVLLHIPALAGKVLRFQKAFLTQLDELLTE |
| | | HRMTWDPAQPPRDLTEAFLAEMEKAKGNPESSFNDENLRIVVADLFSAGM |
| | | VTTSTTLAWGLLLMILHPDVQRRVQQEIDDVIGQVRRPEMGDQAHMPYTT |
| | | AVIHEVQRFGDIVPLGVTHMTSRDIEVQGFRIPKGTTLITNLSSVLKDEAVWE |
| | | KPFRFHPEHFLDAQGHFVKPEAFLPFSAGRRACLGEPLARMELFLFFTSLLQ |
| | | HFSFSVPTGQPRPSHHGVFAFLVSPSPYELCAVPR* |
| 133 | Homo sapiens | ATGGGTTTAGAAGCTTTGGTTCCATTGGCTGTTATCGTTGCTATTTTCTTG |
| | species of | TTGTTGGTTGACTTGATGCATAGAAGGCAAAGATGGGCTGCTAGATATCCA |
| | origin; nucleic | CCAGGTCCATTGCCATTGCCAGGTTTGGGTAATTTGTTGCATGTTGATTTC |
| | acid sequence; | CAAAACACCCCATACTGCTTCGATCAATTGCGTAGAAGATTTGGTGACGTG |
| | 14HC_P450_15 | TTCTCTTTACAATTGGCTTGGACTCCAGTTGTTGTTTTGAATGGTTTGGCT |
| | | GCTGTTAGAGAAGCTTTAGTTACTCATGGTGAAGATACTGCTGATAGACCA |
| | | CCAGTTCCAATCACTCAAATTTTAGGTTTTGGTCCAAGATCTCAGGGTGTT |
| | | TTCTTGGCTAGATATGGTCCAGCTTGGAGAGAACAAAGAAGATTCTCTGTT |
| | | TCTACCTTGAGAAACTTAGGTCTGGGCAAGAAATCTTTGGAACAATGGGTT |
| | | ACTGAAGAGGCTGCTTGTTTGTGTGCTGCTTTTGCTAATCATTCTGGTAGA |
| | | CCATTCAGACCAAATGGTTTGTTGGATAAGGCTGTCTCTAACGTTATTGCT |
| | | TCTTTGACTTGTGGTAGAAGGTTCGAATATGATGACCCAAGATTCTTGAGG |
| | | TTGTTGGATTTGGCTCAAGAAGGTTTGAAAGAAGAGTCTGGTTTCTTGAGA |
| | | GAAGTTTTGAACGCTGTTCCAGTTTTGTTGCACATTCCAGCTTTGGCTGGT |
| | | AAGGTTTTGAGATTTCAAAAGGCTTTCTTGACCCAATTGGATGAGTTGTTG |
| | | ACTGAACATAGAATGACTTGGGACCCAGCTCAACCACCAAGAGATTTGACT |
| | | GAAGCTTTCTTAGCTGAAATGGAAAAGGCTAAGGGTAACCCAGAATCCTCT |
| | | TTTAATGACGAAAACTTGAGAATCGTTGTCGCCGATTTGTTTTCTGCTGGT |
| | | ATGGTTACTACTTCTACTACTTTGGCTTGGGGTTTGTTGTTGATGATATTG |
| | | CATCCAGATGTTCAGAGAAGAGTCCAGCAAGAAATTGATGATGTTATCGGT |
| | | CAAGTTAGAAGGCCAGAAATGGGTGATCAAGCTCATATGCCATATACCACT |
| | | GCTGTTATCCATGAAGTTCAAAGATTCGGTGATATTGTCCCATTGGGTGTT |
| | | ACTCATATGACCTCCAGAGATATAGAAGTTCAGGGTTTTAGAATCCCAAAG |
| | | GGTACTACCTTGATTACCAACTTGTCCTCTGTTTTGAAGGATGAAGCTGTT |
| | | TGGGAAAAGCCATTCAGATTTCATCCAGAACATTTCTTGGATGCCCAAGGT |
| | | CATTTTGTTAAGCCAGAAGCTTTTCTGCCATTTTCAGCAGGTAGAAGGGCT |
| | | TGTTTGGGTGAACCATTAGCTAGAATGGAACTGTTCTTGTTCTTCACCTCT |
| | | TTGCTGCAACATTTCTCTTTCTCTGTTCCAACTGGTCAACCTAGACCATCTC |
| | | ATCATGGTGTTTTCGCTTTCTTGGTTTCTCCATCTCCATATGAATTATGTG |
| | | CCGTTCCAAGATAA |
| 134 | Homo sapiens | MSALGVTVALLVWAAFLLLVSMWRQVHSSWNLPPGPFPLPIIGNLFQLELK |
| | species of | NIPKSFTRLAQRFGPVFTLYVGSQRMVVMHGYKAVKEALLDYKDEFSGRGD |
| | origin; amino | LPAFHAHRDRGIIFNNGPTWKDIRRFSLTTLRNYGMGKQGNESRIQREAHF |
| | acid sequence; | LLEALRKTQGQPFDPTFLIGCAPCNVIADILFRKHFDYNDEKFLRLMYLFNE |
| | 14HC_P450_16 | NFHLLSTPWLQLYNNFPSFLHYLPGSHRKVIKNVAEVKEYVSERVKEHHQS |
| | | LDPNCPRDLTDCLLVEMEKEKHSAERLYTMDGITVTVADLFFAGTETTSTT |
| | | LRYGLLILMKYPEIEEKLHEEIDRVIGPSRIPAIKDRQEMPYMDAVVHEIQRFI |
| | | TLVPSNLPHEATRDTIFRGYLIPKGTVVVPTLDSVLYDNQEFPDPEKFKPEH |
| | | FLNENGKFKYSDYFKPFSTGKRVCAGEGLARMELFLLLCAILQHFNLKPLVD |
| | | PKDIDLSPIHIGFGCIPPRYKLCVIPRS* |
| 135 | Homo sapiens | ATGTCTGCTTTGGGTGTTACTGTTGCTTTGTTGGTTTGGGCTGCTTTCTTG |
| | species of | TTGTTAGTTTCTATGTGGCGTCAAGTTCACTCTTCTTGGAATTTGCCACCA |
| | origin; nucleic | GGTCCATTTCCATTGCCAATTATTGGTAACTTGTTCCAGTTGGAGTTGAAG |
| | acid sequence; | AACATTCCAAAGTCTTTCACCAGATTGGCTCAAAGATTTGGTCCAGTTTTC |
| | 14HC_P450_16 | ACCTTGTATGTCGGTTCTCAAAGAATGGTTGTTATGCATGGTTACAAGGCT |
| | | GTCAAAGAAGCCTTGTTGGATTACAAGGATGAATTTTCTGGTAGAGGTGAT |
| | | TTGCCAGCTTTTCATGCTCATAGAGATAGAGGTATCATCTTCAACAATGGT |
| | | CCAACCTGGAAGGATATCAGAAGATTCTCTTTGACTACCTTGAGGAATTAC |
| | | GGTATGGGTAAGCAAGGTAACGAATCCAGAATCCAAAGAGAAGCCCATTTC |
| | | TTGTTAGAGGCTTTGAGAAAGACTCAAGGTCAACCATTTGATCCCACTTTC |
| | | TTGATTGGTTGTGCTCCATGTAATGTTATCGCCGATATCTTGTTTAGAAAG |
| | | CACTTCGATTACAACGACGAGAAGTTCTTGAGACTGATGTACTTGTTCAAC |
| | | GAGAACTTCCATTTGTTGTCTACTCCATGGTTGCAGCTGTACAACAATTTT |
| | | CCATCTTTCTTGCATTACTTGCCAGGCTCTCATAGAAAGGTTATTAAGAAT |
| | | GTTGCCGAGGTCAAAGAATACGTGTCTGAAAGAGTAAAAGAACACCACCAA |
| | | TCTTTGGACCCAAATTGTCCAAGAGATTTGACTGACTGCTTGTTGGTTGAA |
| | | ATGGAAAAGGAAAAGCACTCTGCTGAAAGGTTGTACACTATGGATGGTATT |
| | | ACTGTTACCGTTGCCGATTTGTTCTTTGCTGGTACTGAAACTACTTCTACC |
| | | ACTTTGAGATACGGCTTGTTGATCTTGATGAAGTACCCAGAAATCGAAGAA |
| | | AAGTTGCACGAAGAAATCGATAGAGTTATTGGCCCATCTAGAATCCCAGCT |
| | | ATTAAGGATAGGCAAGAAATGCCTTATATGGATGCTGTTGTTCACGAAATC |
| | | CAGAGGTTCATTACTTTGGTTCCATCTAACTTGCCACATGAAGCTACTAGA |
| | | GATACCATTTTCAGAGGTTACTTGATCCCAAAGGGTACTGTTGTTGTTCCA |
| | | ACTTTAGATTCCGTCCTGTACGATAATCAAGAGTTTCCAGATCCAGAAAAG |
| | | TTCAAGCCAGAACACTTCTTGAACGAAAATGGCAAGTTCAAGTACTCCGAT |
| | | TACTTCAAGCCATTCTCTACTGGTAAAAGGGTTTGTGCTGGTGAAGGTTTG |
| | | GCTAGAATGGAATTATTCTTGCTGTTGTGCGCTATCTTGCAACACTTTAAT |
| | | TTGAAGCCATTGGTTGACCCAAAGGACATTGATTTGTCCCCAATTCATATT |
| | | GGTTTCGGTTGTATTCCACCAAGGTACAAGTTGTGTGTTATCCCAAGATCT |
| | | TAA |
| 136 | Homo sapiens | MALIPDLAMETWLLLAVSLVLLYLYGTHSHGLFKKLGIPGPTPLPFLGNILSY |
| | species of | HKGFCMFDMECHKKYGKVWGFYDGQQPVLAITDPDMIKTVLVKECYSVFT |
| | origin; amino | NRRPFGPVGFMKSAISIAEDEEWKRLRSLLSPTFTSGKLKEMVPIIAQYGDVL |
| | acid sequence; | VRNLRREAETGKPVTLKDVFGAYSMDVITSTSFGVNIDSLNNPQDPFVENTK |
| | 14HC_P450_17 | KLLRFDFLDPFFLSITVFPFLIPILEVLNICVFPREVTNFLRKSVKRMKESRLE |
| | | DTQKHRVDFLQLMIDSQNSKETESHKALSDLELVAQSIIFIFAGYETTSSVLSF |
| | | IMYELATHPDVQQKLQEEIDAVLPNKAPPTYDTVLQMEYLDMVVNETLRLF |
| | | PIAMRLERVCKKDVEINGMFIPKGVVVMIPSYALHRDPKYWTEPEKFLPERF |
| | | SKKNKDNIDPYIYTPFGSGPRNCIGMRFALMNMKLALIRVLQNFSFKPCKET |
| | | QIPLKLSLGGLLQPEKPVVLKVESRDGTVSGA* |
| 137 | Homo sapiens | ATGGCCTTGATTCCAGATTTGGCTATGGAAACTTGGTTGTTGTTGGCTGTT |
| | species of | TCTTTGGTCTTGTTGTACTTGTACGGTACTCATTCTCATGGCTTGTTTAAG |
| | origin; nucleic | AAGTTGGGTATTCCAGGTCCAACTCCATTGCCATTCTTGGGTAACATTTTG |
| | acid sequence; | TCTTACCATAAGGGTTTCTGCATGTTCGATATGGAATGCCATAAGAAGTAC |
| | 14HC_P450_17 | GGTAAAGTTTGGGGTTTCTACGATGGTCAACAACCTGTTTTGGCTATTACT |
| | | GATCCAGATATGATCAAGACCGTCTTGGTCAAAGAATGCTACTCTGTTTTC |
| | | ACTAACAGAAGGCCATTTGGTCCAGTTGGTTTTATGAAGTCCGCTATTTCT |
| | | ATTGCCGAAGATGAAGAATGGAAGAGGTTGAGATCTTTGTTGTCTCCAACT |
| | | TTCACTTCCGGCAAATTGAAAGAAATGGTTCCAATTATTGCCCAGTACGGT |
| | | GATGTTTTGGTTAGAAACTTGAGAAGAGAAGCCGAAACTGGTAAACCAGTT |
| | | ACTTTGAAAGATGTTTTCGGTGCCTACTCCATGGATGTTATTACTTCTACT |
| | | TCTTTCGGCGTCAACATCGACTCATTGAACAATCCACAAGATCCATTCGTT |
| | | GAGAACACCAAGAAGTTGTTGAGATTCGATTTCTTGGACCCCTTCTTCTTG |
| | | TCCATTACTGTTTTCCCATTCTTGATCCCAATCTTGGAAGTTTTGAACATC |
| | | TGCGTTTTCCCAAGAGAAGTCACCAATTTCTTGAGAAAGTCCGTCAAGAGA |
| | | ATGAAGGAATCCAGATTGGAAGATACCCAAAAGCACAGAGTTGATTTCTTG |
| | | CAATTGATGATCGACTCCCAGAACTCTAAAGAAACTGAATCTCATAAGGCC |
| | | TTGTCCGATTTGGAATTGGTTGCTCAATCCATCATTTTCATTTTCGCTGGT |
| | | TACGAAACCACCTCCTCTGTTTTGTCTTTCATCATGTATGAATTGGCTACCC |
| | | ATCCTGATGTCCAACAAAAGTTGCAAGAAGAAATTGACGCTGTCTTGCCAA |
| | | ACAAAGCTCCACCAACTTATGATACTGTCTTGCAGATGGAATACTTGGATA |
| | | TGGTTGTTAACGAAACCTTGAGGTTGTTCCCAATTGCTATGAGATTGGAAA |
| | | GGGTCTGTAAGAAAGATGTCGAAATCAACGGTATGTTCATCCCAAAAGGTG |
| | | TTGTTGTTATGATTCCATCTTACGCCTTGCATAGAGATCCAAAGTATTGGA |
| | | CTGAACCAGAAAAGTTCTTGCCAGAAAGATTCTCCAAGAAGAACAAGGATA |
| | | ACATCGACCCATATATCTACACTCCATTTGGTTCTGGTCCTAGAAACTGTA |
| | | TCGGTATGAGATTTGCTCTGATGAACATGAAGTTGGCTTTGATTAGAGTGT |
| | | TGCAGAACTTCTCTTTCAAACCCTGTAAAGAAACCCAGATTCCACTGAAGT |
| | | TGTCTTTAGGTGGTTTGTTGCAACCTGAAAAGCCAGTTGTTTTGAAGGTCG |
| | | AATCTAGAGATGGTACTGTTTCTGGTGCTTAA |
| 138 | Bacillus | MTIKEMPQPKTFGELKNLPLLNTDKPVQALMKIADELGEIFKFEAPGRVTRY |
| | megaterium | LSSQRLIKEACDESRFDKNLSQAAKFARDFAGDGLVTSWTHEKNWKKAHNI |
| | species of | LLPSFSQQAMKGYHAMMVDIAVQLVQKWERLNADEHIEVSEDMTRLTLDT |
| | origin; amino | IGLCGFNYRFNSFYRDQPHPFIISMVRAADEVMNKLQRANPDDPAYDENKR |
| | acid sequence; | QFQEDIKVMNDLVDKIIADRKARGEQSDDLLTQMLNGKDPETGEPLDDGNI |
| | 14HC_P450_18 | RYQIITFLIAGHETTSGLLSFALYFLVKNPHVLQKVAEEAARVLVDPVPSYKQ |
| | | VKQLKYVGMVLNEALRLWPTAPAFSLYAKEDTVLGGEYPLEKGDEVMVLIP |
| | | QLHRDKTVWGDDVEEFRPERFENPSAIPQHAFKPFGNGQRACIGQQFALHE |
| | | ATLVLGMMLKHFDFEDHTNYELDIKETLTLKPKGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPRFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHIEIELPKEASYQEGDHLGVIPRNYEGIVNRVTA |
| | | RFGLDASQQIRLEAEEEKLAHLPLAKTVSVEELLQYVELQDPATRTQLRAMA |
| | | AKTVCPPHKVELEALLEKQAYKEQVLAKRLTMLELLEKYPACEMKFSEFIAL |
| | | LPSLRPRYYSISSSPRVDEKQASITVSVVSGPAWSGYGEYKGIASNYLAELQEG |
| | | DTITCFISTPQSEFTLPKDPETPLIMVGPGTGVAPFRGFIQARKQLKEQGQSL |
| | | GEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 139 | Bacillus | ATGACTATAAAAGAGATGCCACAACCTAAGACCTTCGGCGAGCTAAAGAAC |
| | megaterium | CTGCCTCTATTGAATACGGACAAGCCAGTGCAGGCCCTTATGAAAATTGCG |
| | species of | GATGAGCTGGGTGAGATCTTCAAATTTGAAGCGCCAGGGCGTGTGACTAGA |
| | origin; nucleic | TATTTATCCAGCCAAAGGCTGATAAAAGAGGCTTGCGACGAGTCTCGTTTC |
| | acid sequence; | GATAAGAACCTGAGCCAGGCGGCCAAATTTGCCAGGGATTTCGCGGGAGAC |
| | 14HC_P450_18 | GGGTTGGTAACGTCTTGGACTCACGAGAAAAATTGGAAAAAGGCGCATAAT |
| | | ATATTGCTACCTTCCTTCTCCCAACAAGCGATGAAGGGTTACCATGCAATG |
| | | ATGGTTGATATTGCAGTGCAGTTAGTCCAAAAGTGGGAGAGGCTGAATGCT |
| | | GATGAGCACATTGAAGTCTCTGAGGACATGACGCGTCTAACATTGGATACA |
| | | ATTGGCTTATGTGGATTTAACTATAGATTCAACTCTTTCTATAGAGACCAG |
| | | CCTCATCCTTTCATTATCAGCATGGTCCGTGCCGCAGACGAGGTCATGAAT |
| | | AAATTGCAGCGTGCTAACCCAGATGATCCCGCATACGATGAGAACAAAAGG |
| | | CAGTTCCAGGAGGATATCAAAGTAATGAATGATCTTGTCGACAAAATCATA |
| | | GCGGATCGTAAGGCAAGGGGAGAGCAGTCTGACGACTTGCTGACGCAAATG |
| | | CTGAACGGAAAAGATCCTGAAACAGGTGAACCCCTAGACGACGGGAACATC |
| | | CGTTACCAGATAATAACTTTTTTAATTGCCGGGCACGAGACAACCAGTGGA |
| | | CTGCTGTCCTTTGCCCTGTATTTCTTAGTCAAGAACCCTCATGTGTTACAA |
| | | AAAGTCGCCGAGGAAGCAGCTAGAGTCCTAGTAGACCCCGTGCCAAGCTAT |
| | | AAACAGGTTAAGCAACTTAAATACGTTGGGATGGTCCTGAACGAGGCATTA |
| | | AGGTTATGGCCCACCGCACCTGCGTTTTCACTTTATGCGAAAGAAGATACG |
| | | GTTCTGGGTGGCGAGTACCCTCTTGAAAAGGGAGACGAAGTTATGGTTTTG |
| | | ATACCGCAGCTTCATAGGGACAAGACTGTCTGGGGGGATGATGTCGAAGAA |
| | | TTTCGTCCTGAGAGGTTCGAGAATCCTAGTGCTATCCCGCAACACGCGTTC |
| | | AAACCCTTTGGTAACGGACAAAGGGCCTGTATCGGCCAGCAATTCGCCCTA |
| | | CACGAGGCGACACTTGTTTTAGGCATGATGCTAAAGCATTTTGACTTTGAG |
| | | GATCACACGAACTACGAGCTGGATATCAAAGAGACACTTACCTTGAAACCT |
| | | AAGGGTTTCGTCGTCAAAGCTAAAAGTAAGAAGATCCCTTTGGGAGGCATC |
| | | CCGTCACCGTCCACGGAACAATCTGCGAAGAAAGTTAGGAAAAAAGCCGAG |
| | | AATGCACATAACACCCCGTTATTAGTTCTTTACGGGTCTAACATGGGAACG |
| | | GCCGAGGGAACGGCCCGTGACTTGGCAGACATAGCTATGTCAAAAGGGTTT |
| | | GCCCCGCAGGTCGCGACTCTTGATAGCCATGCGGGCAATCTACCGAGGGAG |
| | | GGCGCCGTTTTGATCGTAACTGCATCCTACAATGGTCACCCCCCCGACAACG |
| | | CCAAGCAATTTGTAGACTGGCTTGACCAAGCGTCCGCTGATGAGGTAAAGG |
| | | GCGTGCGTTATAGTGTTTTCGGGTGTGGCGATAAGAATTGGGCTACTACAT |
| | | ACCAAAAGGTTCCACGTTTCATAGATGAAACTCTGGCCGCAAAGGGCGCGG |
| | | AAAACATCGCCGACCGTGGAGAAGCAGATGCTAGTGATGACTTCGAAGGCA |
| | | CCTACGAAGAGTGGCGTGAACACATGTGGTCCGATGTGGCTGCGTATTTTA |
| | | ACTTAGATATCGAGAATTCTGAGGACAACAAAAGTACGCTATCCCTACAGT |
| | | TCGTAGATTCTGCCGCGGACATGCCTTTGGCTAAAATGCATGGAGCATTCT |
| | | CAACAAATGTGGTGGCCTCTAAAGAGCTACAGCAGCCCGGTTCTGCCCGTT |
| | | CCACTCGTCATATCGAAATCGAGTTACCAAAGGAAGCTTCCTATCAAGAGG |
| | | GAGATCACCTGGGGGTTATTCCACGTAATTACGAGGGCATCGTTAACAGGG |
| | | TAACTGCCCGTTTTGGGTTAGATGCCTCACAACAGATACGTCTGGAGGCCG |
| | | AGGAAGAAAAACTAGCTCACTTGCCACTGGCGAAGACCGTCAGCGTCGAGG |
| | | AGCTGCTGCAATACGTTGAACTGCAAGACCCAGCTACTAGGACGCAATTGC |
| | | GTGCTATGGCAGCTAAAACGGTATGTCCACCTCATAAAGTGGAACTTGAGG |
| | | CTCTGCTGGAAAAGCAGGCGTATAAAGAACAAGTGCTGGCAAAAAGGCTAA |
| | | CCATGCTAGAACTGCTTGAGAAGTACCCGGCGTGCGAAATGAAATTCTCCG |
| | | AGTTCATAGCCCTGTTGCCATCACTTCGTCCTAGGTATTACTCAATATCCTC |
| | | ATCTCCAAGGGTTGATGAGAAGCAAGCGAGCATAACTGTTTCCGTCGTCAG |
| | | CGGTCCAGCGTGGAGCGGATACGGTGAGTACAAAGGAATTGCCTCTAACTA |
| | | CCTGGCAGAGTTACAGGAGGGGGACACTATTACGTGCTTCATCTCTACTCC |
| | | GCAATCAGAGTTCACTCTGCCCAAAGATCCTGAAACTCCCCTAATAATGGT |
| | | AGGCCCAGGCACCGGAGTAGCGCCATTCAGAGGTTTTATTCAGGCTAGAAA |
| | | ACAGCTTAAAGAACAGGGCCAATCTTTAGGTGAAGCCCATCTTTACTTTGG |
| | | TTGTCGTTCCCCTCACGAGGACTATTTATACCAGGAGGAACTTGAAAACGC |
| | | ACAGAGTGAAGGAATCATAACACTACATACCGCATTCTCCAGGATGCCGAA |
| | | TCAACCAAAGACTTACGTTCAACACGTCATGGAGCAAGATGGAAAAAAGCT |
| | | GATCGAGTTACTTGATCAAGGTGCGCACTTTTATATCTGCGGGGATGGTTC |
| | | TCAGATGGCTCCTGCGGTAGAGGCCACCCTAATGAAAAGCTATGCAGACGT |
| | | ACACCAGGTATCCGAGGCGGACGCTAGGCTGTGGCTGCAGCAATTAGAGGA |
| | | AAAGGGTCGTTACGCGAAAGATGTGTGGGCGGGATGA |
| 140 | Bacillus | MTIKEMPQPKTFGELKNLPLLNTDKPVQALMKIADELGEIFKFEAPGRVTRY |
| | megaterium | LSSQRLIKEACDESRFDKNLSQGLKFVRDFAGDGLATSWTHEKNWKKAHNI |
| | species of | LLPSFSQQAMKGYHAMMVDIAVQLVQKWERLNADEHIEVPEDMTRLTLDT |
| | origin; amino | IGLCGFNYRFNSFYRDQPHPFITSMVRALDEAMNKQQRANPDDPAYDENKR |
| | acid sequence; | QFQEDIKVMNDLVDKIIADRKASGEQSDDLLTHMLNGKDPETGEPLDDENI |
| | 14HC_P450_19 | RYQIITFLIAGHVTTSGLLSFALYFLVKNPHVLQKAAEEAARVLVDPVPSYKQ |
| | | VKQLKYVGMVLNEALRLWPTAPAFSLYAKEDTVLGGEYPLEKGDELMVLIP |
| | | QLHRDKTIWGDDVEEFRPERFENPSAIPQHAFKPFGNGQRACIGQQFALHE |
| | | ATLVLGMMLKHFDFEDHTNYELDIKETLTLKPEGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 141 | Bacillus | ATGACTATAAAAGAGATGCCACAACCTAAAACCTTTGGCGAGTTAAAGAAC |
| | megaterium | CTGCCACTACTAAATACGGATAAGCCGGTCCAGGCTCTGATGAAAATTGCG |
| | species of | GATGAGCTGGGGGAGATTTTTAAGTTTGAAGCACCGGGAAGGGTTACTCGT |
| | origin; nucleic | TATCTTTCCTCTCAGAGGTTAATCAAAGAGGCATGTGATGAATCAAGATTC |
| | acid sequence; | GACAAGAACCTGTCTCAGGGACTAAAGTTTGTGCGTGACTTTGCCGGTGAT |
| | 14HC_P450_19 | GGCCTTGCCACGTCCTGGACCCATGAGAAGAACTGGAAGAAAGCTCACAAC |
| | | ATCCTACTTCCCTCATTCTCTCAGCAAGCGATGAAGGGCTACCACGCTATGA |
| | | TGGTCGACATCGCTGTCCAGCTGGTACAAAAATGGGAGAGACTTAATGCGG |
| | | ATGAACATATAGAAGTGCCTGAAGATATGACTAGATTGACTTTAGACACCA |
| | | TAGGCCTGTGCGGGTTTAACTACAGGTTCAACAGCTTTTACCGTGACCAGC |
| | | CTCATCCGTTCATTACATCTATGGTACGTGCCCTTGACGAAGCTATGAATA |
| | | AGCAGCAAAGAGCAAATCCCGACGACCCTGCCTATGATGAGAATAAGAGGC |
| | | AGTTCCAGGAGGACATTAAGGTTATGAATGATTTAGTTGATAAGATCATCG |
| | | CGGACAGGAAAGCGTCAGGTGAACAATCTGATGATCTACTGACACATATGT |
| | | TAAATGGAAAAGATCCCGAGACTGGAGAGCCTTTAGACGACGAAAACATAA |
| | | GATATCAAATTATCACCTTCCTGATTGCTGGACACGTAACAACCTCTGGGT |
| | | TACTATCATTTGCCTTATACTTTCTAGTCAAGAACCCTCATGTCCTTCAAA |
| | | AGGCTGCGGAAGAAGCAGCGAGGGTGCTAGTCGACCCGGTCCCCTCTTACA |
| | | AGCAGGTCAAGCAACTGAAATATGTCGGAATGGTCCTGAATGAGGCGCTAA |
| | | GGCTATGGCCGACGGCTCCTGCTTTCAGTCTATACGCCAAGGAAGATACCG |
| | | TCCTGGGAGGTGAATATCCCCTGGAAAAAGGTGATGAACTAATGGTATTAA |
| | | TTCCACAGCTGCACAGGGACAAGACGATTTGGGGAGACGATGTAGAGGAAT |
| | | TCAGACCCGAAAGATTTGAGAACCCCTCTGCAATTCCCCAACATGCGTTTA |
| | | AGCCGTTTGGCAATGGACAGAGAGCCTGCATCGGACAGCAGTTTGCTCTAC |
| | | ACGAAGCGACGCTGGTGCTAGGGATGATGTTAAAGCATTTTGATTTCGAAG |
| | | ACCACACAAACTATGAGCTGGATATAAAAGAGACATTGACCTTGAAGCCAG |
| | | AAGGGTTTGTCGTGAAAGCTAAGTCTAAAAAAATACCTCTGGGAGGGATTC |
| | | CATCCCCGAGTACCGAGCAAAGTGCCAAAAAGGTAAGAAAGAAAGCGGAAA |
| | | ACGCCCACAACACCCCGTTATTAGTTCTTTACGGGTCTAACATGGGAACGG |
| | | CCGAGGGTACAGCCAGGGACCTTGCAGACATTGCTATGTCAAAGGGCTTTG |
| | | CTCCGCAAGTCGCAACCTTAGATTCCCACGCAGGGAACCTGCCGAGAGAAG |
| | | GAGCGGTGCTTATTGTTACCGCTTCATATAATGGTCATCCTCCGGATAACG |
| | | CCAAACAGTTCGTTGACTGGCTGGATCAGGCCAGCGCGGACGAAGTTAAGG |
| | | GGGTTCGTTATAGCGTGTTCGGTTGCGGAGATAAAAATTGGGCCACCACGT |
| | | ATCAGAAAGTACCAGCGTTCATCGACGAAACTTTAGCCGCCAAGGGTGCAG |
| | | AGAACATAGCTGACAGAGGTGAAGCTGACGCCAGTGACGATTTCGAAGGGA |
| | | CATATGAGGAATGGAGAGAGCACATGTGGAGCGACGTCGCCGCTTATTTTA |
| | | ACCTGGATATAGAGAACTCTGAAGACAATAAATCTACTTTGTCTTTGCAGT |
| | | TTGTCGATTCTGCCGCAGACATGCCGCTTGCTAAAATGCACGGCGCCTTCTC |
| | | CACTAATGTCGTGGCGAGCAAGGAATTGCAACAGCCGGGAAGCGCGCGTTC |
| | | CACCCGTCACTTGGAGATTGAGCTGCCCAAGGAGGCTTCATACCAGGAAGG |
| | | CGACCACTTAGGAGTAATACCTAGGAATTATGAAGGTATAGTTAACAGAGT |
| | | CACGGCGCGTTTTGGCCTGGATGCGTCACAGCAGATCCGTCTAGAGGCAGA |
| | | GGAGGAGAAGTTGGCTCATCTTCCCTTGGCGAAGACGGTCAGTGTCGAGGA |
| | | GCTGCTTCAGTATGTCGAGCTGCAAGATCCCGTCACCAGGACGCAACTTCG |
| | | TGCAATGGCCGCAAAGACCGTGTGCCCTCCACACAAAGTGGAACTAGAAGC |
| | | TCTTTTAGAGAAACAAGCGTACAAGGAGCAGGTCCTGGCCAAGCGTTTGAC |
| | | AATGCTTGAACTATTGGAAAAGTACCCAGCGTGTGAAATGAAATTCTCCGA |
| | | ATTTATCGCTCTACTGCCGAGCATCAGACCCCGTTACTACTCTATCTCTTCC |
| | | AGTCCGAGGGTAGATGAAAAGCAGGCATCTATAACAGTCTCCGTCGTCTCT |
| | | GGGGAAGCTTGGAGTGGGTACGGGGAATATAAAGGCATAGCCTCCAATTAC |
| | | CTTGCTGAGCTGCAGGAAGGTGACACAATCACATGTTTCATTTCAACCCCT |
| | | CAAAGTGAATTTACTCTGCCAAAGGATCCGGAGACTCCACTAATCATGGTC |
| | | GGGCCTGGAACTGGTGTAGCGCCTTTTAGAGGTTTCGTCCAAGCCAGAAAG |
| | | CAACTGAAAGAGCAGGGTCAGTCATTAGGCGAAGCCCATTTATACTTTGGA |
| | | TGTAGATCTCCTCATGAAGATTATCTGTACCAGGAAGAACTAGAAAACGCT |
| | | CAAAGCGAAGGGATTATTACACTGCACACGGCCTTCAGCCGTATGCCTAAT |
| | | CAGCCCAAAACCTATGTGCAACATGTAATGGAGCAAGATGGTAAAAAGTTA |
| | | ATTGAGTTGTTAGATCAGGGCGCACATTTTTACATTTGTGGGGATGGCTCT |
| | | CAGATGGCGCCCGCTGTGGAAGCAACCCTGATGAAGAGTTACGCAGATGTA |
| | | CACCAGGTCAGTGAGGCGGATGCAAGGCTGTGGTTACAGCAACTTGAAGAA |
| | | AAGGGCCGTTACGCTAAGGACGTGTGGGGGGATGA |
| 142 | Bacillus | MTIKEMPQPKTFGELKNLPLLNTDKPVQALMKIADELGEIFKFEAPGLVTRY |
| | megaterium | LSSQRLIKEACDESRFDKNLSQGLKFVRDFAGDGLATSWTHEKNWKKAHNI |
| | species of | LLPSFSQQAMKGYHAMMVDIAVQLVQKWERLNADEHIEVPEDMTRLTLDT |
| | origin; amino | IGLCGFNYRFNSFYRDQPHPFITSMVRALDEAMNKQQRANPDDPAYDENKR |
| | acid sequence; | QFQEDIKVMNDLVDKIIADRKASGEQSDDLLTHMLNGKDPETGEPLDDENI |
| | 14HC_P450_20 | RYQIITFLIAGHVTTSGLLSFALYFLVKNPHVLQKAAEEAARVLVDPVPSYKQ |
| | | VKQLKYVGMVLNEALRLWPTAPAFSLYAKEDTVLGGEYPLEKGDELMVLIP |
| | | QLHRDKTIWGDDVEEFRPERFENPSAIPQHAFKPFGNGQRACIGQQFALHE |
| | | ATLVLGMMLKHFDFEDHTNYELDIKETLTLKPEGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 143 | Bacillus | ATGACGATTAAGGAGATGCCCCAGCCAAAAACATTCGGCGAGCTGAAGAAC |
| | megaterium | TTGCCGCTGTTAAATACCGACAAGCCGGTCCAGGCATTGATGAAAATAGCG |
| | species of | GACGAATTAGGAGAAATTTTCAAATTTGAAGCGCCGGGCCTAGTGACAAGA |
| | origin; nucleic | TACTTATCATCACAGAGACTAATCAAGGAAGCGTGTGATGAGAGCCGTTTC |
| | acdi sequence; | GATAAGAACCTAAGCCAGGGATTGAAATTTGTGCGTGATTTCGCGGGTGAT |
| | 14HC_P450_20 | GGTTTGGCTACCTCTTGGACGCACGAAAAGAACTGGAAAAAGGCTCACAAC |
| | | ATATTGCTGCCCTCCTTCAGCCAACAGGCGATGAAGGGCTATCATGCTATG |
| | | ATGGTAGACATTGCGGTTCAGCTGGTACAGAAGTGGGAGAGGCTGAACGCA |
| | | GATGAGCATATTGAGGTCCCTGAAGATATGACAAGGCTGACCCTAGACACT |
| | | ATAGGATTATGCGGCTTTAATTACCGTTTTAATTCATTCTACAGGGATCAA |
| | | CCACATCCATTCATCACATCAATGGTACGTGCATTAGACGAAGCAATGAAC |
| | | AAGCAGCAGAGGGCGAACCCAGATGACCCTGCATATGACGAGAACAAGAGG |
| | | CAATTTCAGGAGGACATCAAGGTAATGAATGACCTTGTTGACAAGATCATC |
| | | GCCGACAGAAAGGCATCCGGGGAACAGTCTGACGATCTGCTAACACACATG |
| | | CTTAACGGCAAAGATCCCGAGACTGGGGAGCCTCTTGACGATGAGAATATC |
| | | CGTTACCAAATCATTACGTTCTTAATCGCGGGGCACGTTACCACATCTGGC |
| | | TTACTTTCATTCGCCTTATATTTCTTAGTAAAGAACCCACACGTCCTGCAA |
| | | AAGGCCGCAGAAGAGGCTGCGCGTGTCCTTGTGGACCCGGTTCCAAGTTAT |
| | | AAACAGGTGAAACAACTAAAGTATGTGGGTATGGTACTAAATGAGGCCCTG |
| | | CGTCTTTGGCCTACCGCCCCTGCCTTTAGTCTATATGCAAAGGAGGACACTG |
| | | TATTAGGTGGGGAATATCCCCTGGAGAAGGGCGATGAGCTAATGGTGCTGA |
| | | TTCCTCAACTACATAGGGATAAAACTATCTGGGGAGATGACGTAGAGGAAT |
| | | TCAGACCTGAAAGGTTTGAAAACCCGAGTGCGATACCTCAACATGCTTTCA |
| | | AACCATTCGGCAATGGTCAGAGGGCATGTATAGGACAACAATTTGCACTAC |
| | | ATGAAGCAACGCTAGTGCTGGGTATGATGCTAAAGCACTTCGATTTTGAGG |
| | | ATCACACTAACTATGAACTAGACATAAAAGAGACTCTTACCCTTAAACCGG |
| | | AAGGGTTCGTAGTGAAAGCGAAGAGTAAAAAAATACCCCTTGGAGGTATA |
| | | CCCTCCCCTAGCACCGAACAGTCAGCTAAGAAAGTGCGTAAAAAGGCGGAG |
| | | AACGCTCACAATACCCCGTTATTAGTTCTTTACGGGTCTAACATGGGAACG |
| | | GCCGAGGGTACAGCCAGGGACCTTGCAGACATTGCTATGTCAAAGGGCTTT |
| | | GCTCCGCAAGTCGCAACCTTAGATTCCCACGCAGGGAACCTGCCGAGAGAA |
| | | GGAGCGGTGCTTATTGTTACCGCTTCATATAATGGTCATCCTCCGGATAAC |
| | | GCCAAACAGTTCGTTGACTGGCTGGATCAGGCCAGCGCGGACGAAGTTAAG |
| | | GGGGTTCGTTATAGCGTGTTCGGTTGCGGAGATAAAAATTGGGCCACCACG |
| | | TATCAGAAAGTACCAGCGTTCATCGACGAAACTTTAGCCGCCAAGGGTGCA |
| | | GAGAACATAGCTGACAGAGGTGAAGCTGACGCCAGTGACGATTTCGAAGGG |
| | | ACATATGAGGAATGGAGAGAGCACATGTGGAGCGACGTCGCCGCTTATTTT |
| | | AACCTGGATATAGAGAACTCTGAAGACAATAAATCTACTTTGTCTTTGCAG |
| | | TTTGTCGATTCTGCCGCAGACATGCCGCTTGCTAAAATGCACGGCGCCTTCT |
| | | CCACTAATGTCGTGGCGAGCAAGGAATTGCAACAGCCGGGAAGCGCGCGTT |
| | | CCACCCGTCACTTGGAGATTGAGCTGCCCAAGGAGGCTTCATACCAGGAAG |
| | | GCGACCACTTAGGAGTAATACCTAGGAATTATGAAGGTATAGTTAACAGAG |
| | | TCACGGCGCGTTTTGGCCTGGATGCGTCACAGCAGATCCGTCTAGAGGCAG |
| | | AGGAGGAGAAGTTGGCTCATCTTCCCTTGGCGAAGACGGTCAGTGTCGAGG |
| | | AGCTGCTTCAGTATGTCGAGCTGCAAGATCCCGTCACCAGGACGCAACTTC |
| | | GTGCAATGGCCGCAAAGACCGTGTGCCCTCCACACAAAGTGGAACTAGAAG |
| | | CTCTTTTAGAGAAACAAGCGTACAAGGAGCAGGTCCTGGCCAAGCGTTTGA |
| | | CAATGCTTGAACTATTGGAAAAGTACCCAGCGTGTGAAATGAAATTCTCCG |
| | | AATTTATCGCTCTACTGCCGAGCATCAGACCCCGTTACTACTCTATCTCTTC |
| | | CAGTCCGAGGGTAGATGAAAAGCAGGCATCTATAACAGTCTCCGTCGTCTC |
| | | TGGGGAAGCTTGGAGTGGGTACGGGGAATATAAAGGCATAGCCTCCAATTA |
| | | CCTTGCTGAGCTGCAGGAAGGTGACACAATCACATGTTTCATTTCAACCCC |
| | | TCAAAGTGAATTTACTCTGCCAAAGGATCCGGAGACTCCACTAATCATGGT |
| | | CGGGCCTGGAACTGGTGTAGCGCCTTTTAGAGGTTTCGTCCAAGCCAGAAA |
| | | GCAACTGAAAGAGCAGGGTCAGTCATTAGGCGAAGCCCATTTATACTTTGG |
| | | ATGTAGATCTCCTCATGAAGATTATCTGTACCAGGAAGAACTAGAAAACGC |
| | | TCAAAGCGAAGGGATTATTACACTGCACACGGCCTTCAGCCGTATGCCTAA |
| | | TCAGCCCAAAACCTATGTGCAACATGTAATGGAGCAAGATGGTAAAAAGTT |
| | | AATTGAGTTGTTAGATCAGGGCGCACATTTTTACATTTGTGGGGATGGCTC |
| | | TCAGATGGCGCCCGCTGTGGAAGCAACCCTGATGAAGAGTTACGCAGATGT |
| | | ACACCAGGTCAGTGAGGCGGATGCAAGGCTGTGGTTACAGCAACTTGAAGA |
| | | AAAGGGCCGTTACGCTAAGGACGTGTGGGCGGGATGA |
| 144 | Bacillus | MTIKEMPQPKTFGELKNLPLLNTDKPVQALMKIADELGEIFKFEAPGLVTRY |
| | megaterium | LSSQRLIKEACDESRFDKNLSQGLKFVRDIAGDGIATSWTHEKNWKKAHNIL |
| | species of | LPSFSQQAMKGYHAMMVDIAVQLVQKWERLNADEHIEVPEDMTRLTLDTI |
| | origin; amino | GLCGFNYRFNSFYRDQPHPFITSMVRALDEAMNKQQRANPDDPAYDENKR |
| | acid sequence; | QFQEDIKVMNDLVDKIIADRKASGEQSDDLLTHMLNGKDPETGEPLDDENI |
| | 14HC_P450_21 | RYQIITFLIAGHVTTSGLLSFALYFLVKNPHVLQKAAEEAARVLVDPVPSYKQ |
| | | VKQLKYVGMVLNEALRLWPTAPAFSLYAKEDTVLGGEYPLEKGDELMVLIP |
| | | QLHRDKTIWGDDVEEFRPERFENPSAIPQHAFKPFGNGQRACIGQQFALHE |
| | | ATLVLGMMLKHFDFEDHTNYELDIKETLTLKPEGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 145 | Bacillus | ATGACTATTAAAGAGATGCCGCAACCTAAAACTTTTGGGGAACTAAAAAAC |
| | megaterium | TTACCACTTCTAAACACAGACAAACCTGTTCAAGCGTTGATGAAGATCGCA |
| | species of | GATGAGCTGGGAGAAATTTTCAAGTTCGAGGCACCGGGGCTGGTAACGCGT |
| | origin; nucleic | TACCTTAGTAGCCAGCGTCTAATTAAAGAAGCGTGTGATGAAAGTAGGTTC |
| | acid sequence; | GACAAGAATTTATCACAGGGGCTTAAGTTCGTCAGGGATATTGCCGGTGAT |
| | 14HC_P450_21 | GGGATCGCTACAAGCTGGACACATGAGAAAAATTGGAAGAAGGCCCATAAT |
| | | ATCCTTCTACCCAGTTTCAGCCAACAGGCTATGAAGGGGTATCATGCAATG |
| | | ATGGTGGATATTGCTGTTCAGTTAGTCCAGAAGTGGGAACGTCTTAATGCG |
| | | GATGAGCACATAGAAGTGCCTGAAGATATGACAAGACTGACGCTTGACACC |
| | | ATAGGCCTGTGTGGGTTCAACTATAGATTCAATAGTTTTTATAGAGACCAA |
| | | CCGCATCCGTTTATCACGTCTATGGTGAGAGCACTGGATGAGGCTATGAAC |
| | | AAGCAACAAAGGGCAAATCCCGATGATCCAGCTTACGACGAGAATAAGCGT |
| | | CAGTTTCAAGAAGACATCAAGGTAATGAATGATCTAGTGGACAAAATTAT |
| | | AGCCGATAGAAAAGCCTCTGGTGAACAGAGCGATGATCTGCTAACACATAT |
| | | GCTGAACGGCAAAGATCCAGAGACTGGAGAGCCTCTGGATGATGAAAATAT |
| | | AAGATACCAAATAATTACTTTCTTAATCGCGGGCCATGTTACTACGTCAGG |
| | | ACTGTTAAGTTTTGCATTGTACTTTCTAGTTAAGAATCCCCACGTTCTGCA |
| | | AAAAGCGGCGGAGGAGGCCGCCAGAGTCTTAGTTGACCCAGTGCCCTCCTA |
| | | CAAACAGGTAAAACAGCTTAAGTATGTGGGTATGGTGTTAAATGAGGCACT |
| | | TCGTTTGTGGCCCACTGCCCCAGCGTTTAGTCTATACGCAAAAGAAGACAC |
| | | CGTCTTAGGAGGTGAATATCCGCTTGAGAAGGGAGATGAATTGATGGTCCT |
| | | AATTCCCCAATTACATAGAGACAAAACCATCTGGGGAGACGACGTTGAAGA |
| | | GTTCAGACCAGAGAGGTTCGAAAACCCTTCTGCCATACCACAGCACGCTTT |
| | | CAAACCTTTCGGAAATGGTCAGAGGGCCTGTATAGGACAACAGTTTGCGCT |
| | | TCATGAAGCAACGCTTGTACTTGGAATGATGCTGAAGCACTTTGACTTTGA |
| | | GGACCACACGAATTACGAATTAGACATCAAGGAGACATTGACACTTAAGCC |
| | | TGAAGGCTTCGTTGTAAAGGCGAAGTCCAAAAAGATCCCACTTGGTGGTAT |
| | | ACCATCACCATCCACCGAACAAAGCGCGAAGAAGGTTAGGAAAAAAGCTGA |
| | | AAATGCTCATAACACCCCGTTATTAGTTCTTTACGGGTCTAACATGGGAAC |
| | | GGCCGAGGGTACAGCCAGGGACCTTGCAGACATTGCTATGTCAAAGGGCTT |
| | | TGCTCCGCAAGTCGCAACCTTAGATTCCCACGCAGGGAACCTGCCGAGAGA |
| | | AGGAGCGGTGCTTATTGTTACCGCTTCATATAATGGTCATCCTCCGGATAA |
| | | CGCCAAACAGTTCGTTGACTGGCTGGATCAGGCCAGCGCGGACGAAGTTAA |
| | | GGGGGTTCGTTATAGCGTGTTCGGTTGCGGAGATAAAAATTGGGCCACCAC |
| | | GTATCAGAAAGTACCAGCGTTCATCGACGAAACTTTAGCCGCCAAGGGTGC |
| | | AGAGAACATAGCTGACAGAGGTGAAGCTGACGCCAGTGACGATTTCGAAGG |
| | | GACATATGAGGAATGGAGAGAGCACATGTGGAGCGACGTCGCCGCTTATTT |
| | | TAACCTGGATATAGAGAACTCTGAAGACAATAAATCTACTTTGTCTTTGCA |
| | | GTTTGTCGATTCTGCCGCAGACATGCCGCTTGCTAAAATGCACGGCGCCTTC |
| | | TCCACTAATGTCGTGGCGAGCAAGGAATTGCAACAGCCGGGAAGCGCGCGT |
| | | TCCACCCGTCACTTGGAGATTGAGCTGCCCAAGGAGGCTTCATACCAGGAA |
| | | GGCGACCACTTAGGAGTAATACCTAGGAATTATGAAGGTATAGTTAACAGA |
| | | GTCACGGCGCGTTTTGGCCTGGATGCGTCACAGCAGATCCGTCTAGAGGCA |
| | | GAGGAGGAGAAGTTGGCTCATCTTCCCTTGGCGAAGACGGTCAGTGTCGAG |
| | | GAGCTGCTTCAGTATGTCGAGCTGCAAGATCCCGTCACCAGGACGCAACTT |
| | | CGTGCAATGGCCGCAAAGACCGTGTGCCCTCCACACAAAGTGGAACTAGAA |
| | | GCTCTTTTAGAGAAACAAGCGTACAAGGAGCAGGTCCTGGCCAAGCGTTTG |
| | | ACAATGCTTGAACTATTGGAAAAGTACCCAGCGTGTGAAATGAAATTCTCC |
| | | GAATTTATCGCTCTACTGCCGAGCATCAGACCCCGTTACTACTCTATCTCTT |
| | | CCAGTCCGAGGGTAGATGAAAAGCAGGCATCTATAACAGTCTCCGTCGTCT |
| | | CTGGGGAAGCTTGGAGTGGGTACGGGGAATATAAAGGCATAGCCTCCAATT |
| | | ACCTTGCTGAGCTGCAGGAAGGTGACACAATCACATGTTTCATTTCAACCC |
| | | CTCAAAGTGAATTTACTCTGCCAAAGGATCCGGAGACTCCACTAATCATGG |
| | | TCGGGCCTGGAACTGGTGTAGCGCCTTTTAGAGGTTTCGTCCAAGCCAGAA |
| | | AGCAACTGAAAGAGCAGGGTCAGTCATTAGGCGAAGCCCATTTATACTTTG |
| | | GATGTAGATCTCCTCATGAAGATTATCTGTACCAGGAAGAACTAGAAAACG |
| | | CTCAAAGCGAAGGGATTATTACACTGCACACGGCCTTCAGCCGTATGCCTA |
| | | ATCAGCCCAAAACCTATGTGCAACATGTAATGGAGCAAGATGGTAAAAAGT |
| | | TAATTGAGTTGTTAGATCAGGGCGCACATTTTTACATTTGTGGGGATGGCT |
| | | CTCAGATGGCGCCCGCTGTGGAAGCAACCCTGATGAAGAGTTACGCAGATG |
| | | TACACCAGGTCAGTGAGGCGGATGCAAGGCTGTGGTTACAGCAACTTGAAG |
| | | AAAAGGGCCGTTACGCTAAGGACGTGTGGGCGGGATGA |
| 146 | Bacillus | MTIKEMPQPKTFGELKNLPLLNTDKPVQALMKIADELGEIFKFEAPGLVTRY |
| | megaterium | LSSQRLIKEACDGSRFDKNLSQGLKFVRDIAGDGLATSWTHEKNWKKAHNI |
| | species of | LLPSFSQQAMKGYHAMMVDIAVQLVQKWERLNADEHIEVPEDMTRLTLDT |
| | origin; amino | IGLCGFNYRFNSFYRDQPHPFITSMVRALDEAMNKQQRANPDDPAYDENKR |
| | acid sequence; | QFQEDIKVMNDLVDKIIADRKASGEQSDDLLTHMLNGKDPETGEPLDDENI |
| | 14HC_P450_22 | RYQIITFLIAGHVTTSGLLSFALYFLVKNPHVLQKAAEEAARVLVDPVPSYKQ |
| | | VKQLKYVGMVLNEALRLWPTAPAFSLYAKEDTVLGGEYPLEKGDELMVLIP |
| | | QLHRDKTIWGDDVEEFRPERFENPSAIPQHAFKPFGNGQRACIGQQFALHE |
| | | ATLVLGMMLKHFDFEDHTNYELDIKETLTLKPEGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 147 | Bacillus | ATGACGATAAAGGAGATGCCCCAACCCAAGACCTTTGGAGAGCTAAAGAAT |
| | megaterium | TTGCCCCTTCTGAACACTGATAAGCCGGTCCAGGCACTAATGAAGATTGCA |
| | species of | GACGAATTGGGGGAAATATTTAAGTTTGAAGCTCCGGGTTTGGTTACAAGG |
| | origin; nucleic | TACCTTAGCAGCCAACGTCTAATCAAGGAAGCGTGCGATGGAAGTCGTTTC |
| | acid sequence; | GACAAAAACCTAAGCCAAGGCTTAAAGTTCGTGAGAGACATAGCCGGGGAC |
| | 14HC_P450_22 | GGCTTAGCAACCAGTTGGACCCACGAAAAGAATTGGAAGAAGGCACATAAT |
| | | ATACTGCTTCCGAGCTTTTCACAACAGGCTATGAAGGGATATCATGCGATG |
| | | ATGGTCGATATAGCGGTACAACTTGTACAAAAATGGGAGCGTCTAAATGCT |
| | | GACGAGCATATCGAGGTGCCTGAAGACATGACTAGACTGACGCTTGATACC |
| | | ATTGGGCTGTGCGGCTTTAACTATAGATTCAACAGCTTTTATCGTGACCAA |
| | | CCACACCCCTTTATTACCAGCATGGTTAGAGCGCTAGACGAGGCCATGAAT |
| | | AAGCAGCAAAGAGCAAATCCCGACGATCCAGCATATGACGAAAATAAGCGT |
| | | CAGTTTCAAGAAGACATAAAGGTAATGAATGACCTGGTCGATAAAATAAT |
| | | TGCTGATAGAAAGGCCTCAGGGGAGCAGTCCGACGATTTACTTACCCACAT |
| | | GCTGAACGGTAAGGATCCTGAAACCGGGGAACCACTAGACGACGAAAACAT |
| | | AAGGTACCAAATTATTACGTTCTTGATCGCCGGACACGTTACCACTTCTGG |
| | | CTTACTATCCTTTGCGTTGTACTTTCTTGTTAAGAATCCACACGTTCTACA |
| | | GAAAGCCGCCGAGGAGGCGGCACGTGTCCTAGTTGACCCCGTACCGTCCTA |
| | | TAAACAGGTTAAACAACTGAAATACGTCGGCATGGTACTGAACGAAGCTCT |
| | | AAGGCTATGGCCCACCGCCCCAGCCTTCTCCCTTTATGCGAAGGAGGACACC |
| | | GTCTTAGGAGGAGAGTACCCATTGGAAAAAGGCGACGAACTAATGGTGTTA |
| | | ATTCCCCAGCTTCACAGAGATAAGACGATATGGGGAGATGACGTGGAGGAA |
| | | TTCCGTCCGGAAAGGTTCGAAAATCCATCCGCAATACCACAGCACGCCTTC |
| | | AAGCCTTTTGGGAATGGACAAAGGGCGTGTATAGGGCAACAATTCGCTTTA |
| | | CATGAAGCGACGCTAGTGCTAGGAATGATGCTGAAACACTTCGATTTCGAG |
| | | GATCACACCAATTATGAGCTGGACATAAAAGAAACCCTGACTCTAAAACCG |
| | | GAGGGATTCGTGGTGAAAGCCAAGTCCAAGAAAATTCCTCTTGGTGGTATA |
| | | CCGTCACCCTCTACGGAGCAATCTGCGAAAAAAGTACGTAAAAAAGCGGAG |
| | | AACGCGCATAACACCCCGTTATTAGTTCTTTACGGGTCTAACATGGGAACG |
| | | GCCGAGGGTACAGCCAGGGACCTTGCAGACATTGCTATGTCAAAGGGCTTT |
| | | GCTCCGCAAGTCGCAACCTTAGATTCCCACGCAGGGAACCTGCCGAGAGAA |
| | | GGAGCGGTGCTTATTGTTACCGCTTCATATAATGGTCATCCTCCGGATAAC |
| | | GCCAAACAGTTCGTTGACTGGCTGGATCAGGCCAGCGCGGACGAAGTTAAG |
| | | GGGGTTCGTTATAGCGTGTTCGGTTGCGGAGATAAAAATTGGGCCACCACG |
| | | TATCAGAAAGTACCAGCGTTCATCGACGAAACTTTAGCCGCCAAGGGTGCA |
| | | GAGAACATAGCTGACAGAGGTGAAGCTGACGCCAGTGACGATTTCGAAGGG |
| | | ACATATGAGGAATGGAGAGAGCACATGTGGAGCGACGTCGCCGCTTATTTT |
| | | AACCTGGATATAGAGAACTCTGAAGACAATAAATCTACTTTGTCTTTGCAG |
| | | TTTGTCGATTCTGCCGCAGACATGCCGCTTGCTAAAATGCACGGCGCCTTCT |
| | | CCACTAATGTCGTGGCGAGCAAGGAATTGCAACAGCCGGGAAGCGCGCGTT |
| | | CCACCCGTCACTTGGAGATTGAGCTGCCCAAGGAGGCTTCATACCAGGAAG |
| | | GCGACCACTTAGGAGTAATACCTAGGAATTATGAAGGTATAGTTAACAGAG |
| | | TCACGGCGCGTTTTGGCCTGGATGCGTCACAGCAGATCCGTCTAGAGGCAG |
| | | AGGAGGAGAAGTTGGCTCATCTTCCCTTGGCGAAGACGGTCAGTGTCGAGG |
| | | AGCTGCTTCAGTATGTCGAGCTGCAAGATCCCGTCACCAGGACGCAACTTC |
| | | GTGCAATGGCCGCAAAGACCGTGTGCCCTCCACACAAAGTGGAACTAGAAG |
| | | CTCTTTTAGAGAAACAAGCGTACAAGGAGCAGGTCCTGGCCAAGCGTTTGA |
| | | CAATGCTTGAACTATTGGAAAAGTACCCAGCGTGTGAAATGAAATTCTCCG |
| | | AATTTATCGCTCTACTGCCGAGCATCAGACCCCGTTACTACTCTATCTCTTC |
| | | CAGTCCGAGGGTAGATGAAAAGCAGGCATCTATAACAGTCTCCGTCGTCTC |
| | | TGGGGAAGCTTGGAGTGGGTACGGGGAATATAAAGGCATAGCCTCCAATTA |
| | | CCTTGCTGAGCTGCAGGAAGGTGACACAATCACATGTTTCATTTCAACCCC |
| | | TCAAAGTGAATTTACTCTGCCAAAGGATCCGGAGACTCCACTAATCATGGT |
| | | CGGGCCTGGAACTGGTGTAGCGCCTTTTAGAGGTTTCGTCCAAGCCAGAAA |
| | | GCAACTGAAAGAGCAGGGTCAGTCATTAGGCGAAGCCCATTTATACTTTGG |
| | | ATGTAGATCTCCTCATGAAGATTATCTGTACCAGGAAGAACTAGAAAACGC |
| | | TCAAAGCGAAGGGATTATTACACTGCACACGGCCTTCAGCCGTATGCCTAA |
| | | TCAGCCCAAAACCTATGTGCAACATGTAATGGAGCAAGATGGTAAAAAGTT |
| | | AATTGAGTTGTTAGATCAGGGCGCACATTTTTACATTTGTGGGGATGGCTC |
| | | TCAGATGGCGCCCGCTGTGGAAGCAACCCTGATGAAGAGTTACGCAGATGT |
| | | ACACCAGGTCAGTGAGGCGGATGCAAGGCTGTGGTTACAGCAACTTGAAGA |
| | | AAAGGGCCGTTACGCTAAGGACGTGTGGGCGGGATGA |
| 148 | Bacillus | MTIKEMPQPKTFGELKNLPLLNTDKPVQALMKIADELGEIFKFEAPGLVTRY |
| | megaterium | LSSQRLIKEACDESRFDKNLSQGLKFVRDIAGDGLVTSWTHEKNWKKAHNI |
| | species of | LLPSFSQQAMKGYHAMMVDIAVQLVQKWERLNADEHIEVPEDMTRLTLDT |
| | origin; amino | IGLCGFNYRFNSFYRDQPHPFITSMVRALDEAMNKQQRANPDDPAYDENKR |
| | acid sequence; | QFQEDIKVMNDLVDKIIADRKASGEQSDDLLTHMLNGKDPETGEPLDDENI |
| | 14HC_P450_23 | RYQIITFLIAGHVTTSGLLSFALYFLVKNPHVLQKAAEEAARVLVDPVPSYKQ |
| | | VKQLKYVGMVLNEALRLWPTAPAFSLYAKEDTVLGGEYPLEKGDELMVLIP |
| | | QLHRDKTIWGDDVEEFRPERFENPSAIPQHAFKPFGNGQRACIGQQFALHE |
| | | ATLVLGMMLKHFDFEDHTNYELDIKETLTLKPEGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 149 | Bacillus | ATGACAATTAAGGAAATGCCACAACCGAAAACTTTTGGTGAGCTGAAGAAT |
| | megaterium | TTACCTCTGCTTAACACGGACAAACCTGTGCAAGCCTTAATGAAAATTGCG |
| | species of | GACGAGTTAGGAGAAATTTTCAAGTTTGAGGCACCTGGATTGGTGACTCGT |
| | origin; nucleic | TATCTAAGCAGTCAAAGGCTAATTAAGGAAGCTTGCGACGAGAGCAGATTT |
| | acid sequence; | GATAAAAACCTGTCACAAGGGTTGAAATTTGTCCGTGATATCGCAGGCGAT |
| | 14HC_P450_23 | GGTCTTGTCACTAGTTGGACGCATGAAAAGAACTGGAAGAAGGCACATAAT |
| | | ATTCTATTACCGTCTTTCAGCCAGCAGGCGATGAAAGGGTATCACGCAATG |
| | | ATGGTCGACATCGCAGTCCAGCTTGTTCAGAAATGGGAGAGGTTGAACGCC |
| | | GACGAGCATATAGAGGTACCTGAGGACATGACCAGATTGACATTGGATACA |
| | | ATAGGGCTATGTGGCTTTAATTATCGTTTTAACTCTTTCTATAGAGACCAA |
| | | CCCCATCCATTCATCACCTCCATGGTAAGAGCTCTAGATGAAGCGATGAAC |
| | | AAACAACAAAGAGCTAATCCCGATGACCCTGCCTACGACGAGAACAAACGT |
| | | CAGTTTCAAGAGGACATAAAAGTCATGAATGATCTTGTGGACAAGATTATT |
| | | GCAGATAGGAAAGCATCCGGAGAACAGTCTGACGACCTTTTAACCCATATG |
| | | CTTAACGGCAAGGACCCCGAAACGGGTGAACCTCTTGACGACGAGAATATA |
| | | AGGTATCAGATAATTACCTTCTTAATAGCTGGTCACGTCACGACCTCCGGC |
| | | TTGTTGTCCTTTGCGCTATATTTTCTAGTTAAAAACCCGCATGTTTTACAG |
| | | AAGGCAGCTGAGGAAGCTGCGAGGGTGCTAGTTGATCCCGTCCCGTCATAT |
| | | AAGCAGGTGAAACAGCTGAAGTATGTGGGAATGGTACTTAACGAGGCTCTA |
| | | CGTTTATGGCCGACCGCGCCCGCGTTCTCTTTATACGCGAAGGAAGATACC |
| | | GTGCTAGGCGGCGAATATCCCTTAGAGAAGGGTGACGAACTTATGGTTCTG |
| | | ATACCTCAGTTACATCGTGATAAAACCATCTGGGGAGACGACGTCGAAGAA |
| | | TTCAGGCCCGAGAGGTTTGAGAACCCGTCTGCAATCCCCCAACATGCGTTC |
| | | AAACCGTTCGGAAACGGCCAAAGAGCGTGTATTGGGCAGCAATTCGCCTTG |
| | | CACGAGGCAACTTTGGTCTTGGGCATGATGCTTAAGCACTTCGACTTTGAA |
| | | GATCATACGAACTATGAGTTAGACATAAAGGAGACGTTGACCCTAAAACCT |
| | | GAAGGCTTTGTAGTTAAAGCCAAGTCCAAGAAGATCCCGCTGGGAGGAATC |
| | | CCGTCCCCGTCCACCGAACAATCCGCAAAGAAAGTGAGAAAAAAAGCTGAA |
| | | AATGCACACAATACCCCGTTATTAGTTCTTTACGGGTCTAACATGGGAACG |
| | | GCCGAGGGTACAGCCAGGGACCTTGCAGACATTGCTATGTCAAAGGGCTTT |
| | | GCTCCGCAAGTCGCAACCTTAGATTCCCACGCAGGGAACCTGCCGAGAGAA |
| | | GGAGCGGTGCTTATTGTTACCGCTTCATATAATGGTCATCCTCCGGATAAC |
| | | GCCAAACAGTTCGTTGACTGGCTGGATCAGGCCAGCGCGGACGAAGTTAAG |
| | | GGGGTTCGTTATAGCGTGTTCGGTTGCGGAGATAAAAATTGGGCCACCACG |
| | | TATCAGAAAGTACCAGCGTTCATCGACGAAACTTTAGCCGCCAAGGGTGCA |
| | | GAGAACATAGCTGACAGAGGTGAAGCTGACGCCAGTGACGATTTCGAAGGG |
| | | ACATATGAGGAATGGAGAGAGCACATGTGGAGCGACGTCGCCGCTTATTTT |
| | | AACCTGGATATAGAGAACTCTGAAGACAATAAATCTACTTTGTCTTTGCAG |
| | | TTTGTCGATTCTGCCGCAGACATGCCGCTTGCTAAAATGCACGGCGCCTTCT |
| | | CCACTAATGTCGTGGCGAGCAAGGAATTGCAACAGCCGGGAAGCGCGCGTT |
| | | CCACCCGTCACTTGGAGATTGAGCTGCCCAAGGAGGCTTCATACCAGGAAG |
| | | GCGACCACTTAGGAGTAATACCTAGGAATTATGAAGGTATAGTTAACAGAG |
| | | TCACGGCGCGTTTTGGCCTGGATGCGTCACAGCAGATCCGTCTAGAGGCAG |
| | | AGGAGGAGAAGTTGGCTCATCTTCCCTTGGCGAAGACGGTCAGTGTCGAGG |
| | | AGCTGCTTCAGTATGTCGAGCTGCAAGATCCCGTCACCAGGACGCAACTTC |
| | | GTGCAATGGCCGCAAAGACCGTGTGCCCTCCACACAAAGTGGAACTAGAAG |
| | | CTCTTTTAGAGAAACAAGCGTACAAGGAGCAGGTCCTGGCCAAGCGTTTGA |
| | | CAATGCTTGAACTATTGGAAAAGTACCCAGCGTGTGAAATGAAATTCTCCG |
| | | AATTTATCGCTCTACTGCCGAGCATCAGACCCCGTTACTACTCTATCTCTTC |
| | | CAGTCCGAGGGTAGATGAAAAGCAGGCATCTATAACAGTCTCCGTCGTCTC |
| | | TGGGGAAGCTTGGAGTGGGTACGGGGAATATAAAGGCATAGCCTCCAATTA |
| | | CCTTGCTGAGCTGCAGGAAGGTGACACAATCACATGTTTCATTTCAACCCC |
| | | TCAAAGTGAATTTACTCTGCCAAAGGATCCGGAGACTCCACTAATCATGGT |
| | | CGGGCCTGGAACTGGTGTAGCGCCTTTTAGAGGTTTCGTCCAAGCCAGAAA |
| | | GCAACTGAAAGAGCAGGGTCAGTCATTAGGCGAAGCCCATTTATACTTTGG |
| | | ATGTAGATCTCCTCATGAAGATTATCTGTACCAGGAAGAACTAGAAAACGC |
| | | TCAAAGCGAAGGGATTATTACACTGCACACGGCCTTCAGCCGTATGCCTAA |
| | | TCAGCCCAAAACCTATGTGCAACATGTAATGGAGCAAGATGGTAAAAAGTT |
| | | AATTGAGTTGTTAGATCAGGGCGCACATTTTTACATTTGTGGGGATGGCTC |
| | | TCAGATGGCGCCCGCTGTGGAAGCAACCCTGATGAAGAGTTACGCAGATGT |
| | | ACACCAGGTCAGTGAGGCGGATGCAAGGCTGTGGTTACAGCAACTTGAAGA |
| | | AAAGGGCCGTTACGCTAAGGACGTGTGGGCGGGATGA |
| 150 | Bacillus | MTIKEMPQPKTFGELKNLPLLNTDKPVQALMKIADELGEIFKFEAPGRVTRY |
| | megaterium | LSSQRLIKEACDESRFDKNLSQAAKFARDFAGDGLVTSWTHEKNWKKAHNI |
| | species of | LLPSFSQQAMKGYHAMMVDIAVQLVQKWERLNADEHIEVSEDMTRLTLDT |
| | origin; amino | IGLCGFNYRFNSFYRDQPHPFIISAVRAADEVMNKLQRANPDDPAYDENKR |
| | acid sequence; | QFQEDIKVMNDLVDKIIADRKARGEQSDDLLTQMLNGKDPETGEPLDDGNI |
| | 14HC_P450_24 | RYQIITFLIAGHETTSGLLSFALYFLVKNPHVLQKVAEEAARVLVDPVPSYKQ |
| | | VKQLKYVGMVLNEALRLWPTAPAFSLYAKEDTVLGGEYPLEKGDEVMVLIP |
| | | QLHRDKTVWGDDVEEFRPERFENPSAIPQHAFKPFGNGQRACIGQQFALHE |
| | | ATLVLGMMLKHFDFEDHTNYELDIKETLTLKPKGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 151 | Bacillus | ATGACGATAAAAGAAATGCCCCAGCCAAAGACATTTGGGGAGCTTAAGAAT |
| | megaterium | CTACCATTGCTAAACACCGATAAACCGGTGCAAGCACTTATGAAAATTGCT |
| | species of | GACGAACTAGGTGAAATATTCAAGTTTGAAGCTCCTGGCAGAGTAACAAGG |
| | origin; nucleic | TACTTATCATCTCAAAGATTAATCAAGGAGGCCTGCGACGAGTCTAGATTC |
| | acid sequence; | GACAAAAATCTATCTCAGGCAGCCAAATTTGCTAGAGATTTCGCCGGGGAT |
| | 14HC_P450_24 | GGTTTGGTGACTTCCTGGACACATGAGAAGAATTGGAAGAAAGCTCATAAC |
| | | ATATTACTACCATCCTTCAGTCAACAAGCGATGAAGGGATATCATGCCATG |
| | | ATGGTTGATATCGCTGTTCAGTTGGTCCAAAAATGGGAACGTCTGAACGCA |
| | | GATGAACATATCGAGGTCTCTGAGGATATGACTAGGTTAACACTGGACACT |
| | | ATCGGTTTGTGTGGATTTAACTATCGTTTTAATAGCTTCTATAGAGACCAA |
| | | CCCCACCCATTCATCATATCTGCTGTACGTGCGGCAGATGAGGTCATGAAT |
| | | AAGCTGCAGAGGGCTAATCCGGACGACCCAGCGTACGACGAAAACAAGAGA |
| | | CAGTTTCAAGAAGACATAAAGGTGATGAATGACCTTGTAGACAAGATCATA |
| | | GCTGATAGAAAGGCCCGTGGAGAACAATCCGATGATCTGCTTACGCAGATG |
| | | TTGAATGGCAAGGATCCCGAGACAGGAGAGCCACTGGATGATGGGAACATT |
| | | AGATACCAGATAATTACATTCCTAATCGCGGGGCACGAGACCACGTCTGGC |
| | | CTACTATCATTCGCTCTTTATTTTTTGGTGAAAAATCCACATGTCTTACAG |
| | | AAAGTCGCGGAGGAGGCGGCCAGAGTTCTAGTTGACCCCGTTCCAAGCTAC |
| | | AAGCAAGTAAAGCAACTTAAATATGTTGGAATGGTCCTTAACGAAGCATTG |
| | | AGACTATGGCCGACCGCCCCCGCATTCTCATTATACGCAAAGGAAGATACC |
| | | GTTCTGGGGGGGGAATACCCTCTAGAGAAAGGTGACGAAGTAATGGTCCTA |
| | | ATCCCCCAGCTTCATCGTGACAAAACAGTATGGGGCGACGACGTCGAAGAA |
| | | TTCAGGCCTGAAAGATTCGAGAATCCGTCAGCAATACCTCAGCACGCTTTC |
| | | AAGCCCTTTGGGAATGGTCAGAGGGCGTGTATAGGGCAACAGTTCGCATTA |
| | | CATGAAGCGACACTGGTGCTGGGAATGATGCTTAAGCATTTCGATTTTGAG |
| | | GACCACACGAATTACGAACTAGACATTAAGGAAACACTGACCCTAAAACCA |
| | | AAGGGCTTTGTCGTGAAGGCAAAAAGCAAGAAAATCCCGTTGGGTGGCATT |
| | | CCCTCTCCTAGCACAGAGCAAAGTGCCAAAAAGGTTAGAAAGAAAGCGGAG |
| | | AATGCCCATAACACCCCGTTATTAGTTCTTTACGGGTCTAACATGGGAACG |
| | | GCCGAGGGTACAGCCAGGGACCTTGCAGACATTGCTATGTCAAAGGGCTTT |
| | | GCTCCGCAAGTCGCAACCTTAGATTCCCACGCAGGGAACCTGCCGAGAGAA |
| | | GGAGCGGTGCTTATTGTTACCGCTTCATATAATGGTCATCCTCCGGATAAC |
| | | GCCAAACAGTTCGTTGACTGGCTGGATCAGGCCAGCGCGGACGAAGTTAAG |
| | | GGGGTTCGTTATAGCGTGTTCGGTTGCGGAGATAAAAATTGGGCCACCACG |
| | | TATCAGAAAGTACCAGCGTTCATCGACGAAACTTTAGCCGCCAAGGGTGCA |
| | | GAGAACATAGCTGACAGAGGTGAAGCTGACGCCAGTGACGATTTCGAAGGG |
| | | ACATATGAGGAATGGAGAGAGCACATGTGGAGCGACGTCGCCGCTTATTTT |
| | | AACCTGGATATAGAGAACTCTGAAGACAATAAATCTACTTTGTCTTTGCAG |
| | | TTTGTCGATTCTGCCGCAGACATGCCGCTTGCTAAAATGCACGGCGCCTTCT |
| | | CCACTAATGTCGTGGCGAGCAAGGAATTGCAACAGCCGGGAAGCGCGCGTT |
| | | CCACCCGTCACTTGGAGATTGAGCTGCCCAAGGAGGCTTCATACCAGGAAG |
| | | GCGACCACTTAGGAGTAATACCTAGGAATTATGAAGGTATAGTTAACAGAG |
| | | TCACGGCGCGTTTTGGCCTGGATGCGTCACAGCAGATCCGTCTAGAGGCAG |
| | | AGGAGGAGAAGTTGGCTCATCTTCCCTTGGCGAAGACGGTCAGTGTCGAGG |
| | | AGCTGCTTCAGTATGTCGAGCTGCAAGATCCCGTCACCAGGACGCAACTTC |
| | | GTGCAATGGCCGCAAAGACCGTGTGCCCTCCACACAAAGTGGAACTAGAAG |
| | | CTCTTTTAGAGAAACAAGCGTACAAGGAGCAGGTCCTGGCCAAGCGTTTGA |
| | | CAATGCTTGAACTATTGGAAAAGTACCCAGCGTGTGAAATGAAATTCTCCG |
| | | AATTTATCGCTCTACTGCCGAGCATCAGACCCCGTTACTACTCTATCTCTTC |
| | | CAGTCCGAGGGTAGATGAAAAGCAGGCATCTATAACAGTCTCCGTCGTCTC |
| | | TGGGGAAGCTTGGAGTGGGTACGGGGAATATAAAGGCATAGCCTCCAATTA |
| | | CCTTGCTGAGCTGCAGGAAGGTGACACAATCACATGTTTCATTTCAACCCC |
| | | TCAAAGTGAATTTACTCTGCCAAAGGATCCGGAGACTCCACTAATCATGGT |
| | | CGGGCCTGGAACTGGTGTAGCGCCTTTTAGAGGTTTCGTCCAAGCCAGAAA |
| | | GCAACTGAAAGAGCAGGGTCAGTCATTAGGCGAAGCCCATTTATACTTTGG |
| | | ATGTAGATCTCCTCATGAAGATTATCTGTACCAGGAAGAACTAGAAAACGC |
| | | TCAAAGCGAAGGGATTATTACACTGCACACGGCCTTCAGCCGTATGCCTAA |
| | | TCAGCCCAAAACCTATGTGCAACATGTAATGGAGCAAGATGGTAAAAAGTT |
| | | AATTGAGTTGTTAGATCAGGGCGCACATTTTTACATTTGTGGGGATGGCTC |
| | | TCAGATGGCGCCCGCTGTGGAAGCAACCCTGATGAAGAGTTACGCAGATGT |
| | | ACACCAGGTCAGTGAGGCGGATGCAAGGCTGTGGTTACAGCAACTTGAAGA |
| | | AAAGGGCCGTTACGCTAAGGACGTGTGGGCGGGATGA |
| 152 | Bacillus | MTIKEMPQPKTFGELKNLPLLNTDKPVQALMKIADELGEIFKFEAPGRVTRY |
| | megaterium | LSSQRLIKEACDESRFDKNLSQAAKFARDFAGDGLVTSWTHEKNWKKAHNI |
| | species of | LLPSFSQQAMKGYHAMMVDIAVQLVQKWERLNADEHIEVSEDMTRLTLDT |
| | origin; amino | IGLCGFNYRFNSFYRDQPHPFIISAVRAADEVMNKLQRANPDDPAYDENKR |
| | acid sequence; | QFQEDIKVMNDLVDKIIADRKARGEQSDDLLTQMLNGKDPETGEPLDDGNI |
| | 14HC_P450_25 | RYQIIAFLIAGHETTSGLLSFALYFLVKNPHVLQKVAEEAARVLVDPVPSYKQ |
| | | VKQLKYVGMVLNEALRLWPTAPAFSLYAKEDTVLGGEYPLEKGDEVMVLIP |
| | | QLHRDKTVWGDDVEEFRPERFENPSAIPQHAFKPFGNGQRACIGQQFALHE |
| | | ATLVLGMMLKHFDFEDHTNYELDIKETLTLKPKGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 153 | Bacillus | ATGACTATTAAAGAAATGCCCCAACCCAAGACGTTTGGCGAGTTAAAAAAC |
| | megaterium | CTGCCGCTATTAAATACGGATAAGCCGGTCCAGGCACTAATGAAGATCGCT |
| | species of | GATGAGCTGGGCGAAATCTTCAAGTTCGAGGCCCCAGGAAGAGTGACTAGG |
| | origin; nucleic | TATCTTAGCTCACAGAGACTGATAAAAGAAGCGTGTGACGAGTCTAGATTC |
| | acid sequence; | GACAAGAATTTATCCCAAGCGGCCAAGTTTGCGCGTGATTTTGCGGGTGAT |
| | 14HC_P450_25 | GGGCTGGTGACGTCCTGGACTCATGAGAAGAACTGGAAGAAAGCCCATAAC |
| | | ATTTTGCTGCCAAGTTTCTCTCAGCAAGCGATGAAGGGTTACCATGCGATG |
| | | ATGGTTGATATCGCTGTTCAATTGGTACAAAAATGGGAGAGATTAAACGCG |
| | | GATGAGCATATCGAAGTCAGCGAAGACATGACTAGACTTACGCTTGACACT |
| | | ATAGGACTTTGCGGTTTTAACTATAGATTTAATTCCTTTTACCGTGATCAA |
| | | CCCCATCCCTTCATAATCTCAGCGGTAAGAGCTGCGGACGAGGTCATGAAT |
| | | AAGCTGCAGCGTGCGAACCCTGACGATCCTGCGTACGATGAGAATAAAAGG |
| | | CAATTTCAAGAAGACATCAAAGTTATGAACGACTTAGTTGACAAGATTATT |
| | | GCAGATAGAAAAGCGCGTGGAGAACAGTCCGACGATCTTTTAACGCAGATG |
| | | TTAAATGGAAAAGACCCAGAAACGGGAGAGCCTCTAGACGACGGTAATATT |
| | | CGTTATCAGATTATAGCTTTCCTGATAGCAGGGCACGAGACCACTTCAGGC |
| | | TTATTAAGCTTCGCCCTGTACTTTCTAGTCAAGAACCCCCACGTCCTGCAGA |
| | | AGGTAGCTGAAGAGGCTGCAAGGGTCCTTGTAGATCCTGTTCCTTCTTATA |
| | | AACAGGTCAAACAATTAAAGTACGTTGGGATGGTTCTTAACGAAGCGTTAC |
| | | GTTTATGGCCCACCGCTCCTGCTTTTTCTTTGTATGCGAAAGAAGACACCG |
| | | TTCTTGGAGGTGAATATCCTCTAGAGAAAGGAGATGAGGTTATGGTTTTAA |
| | | TTCCGCAGCTTCACCGTGATAAAACGGTTTGGGGAGATGACGTCGAGGAAT |
| | | TCCGTCCCGAAAGGTTCGAGAATCCGAGCGCGATCCCCCAACACGCATTCA |
| | | AGCCCTTCGGGAATGGGCAACGTGCCTGTATAGGTCAGCAATTCGCCCTAC |
| | | ACGAGGCCACTCTTGTTCTGGGCATGATGTTGAAACATTTTGACTTTGAAG |
| | | ATCACACTAATTATGAGTTGGATATCAAGGAAACGTTGACACTGAAACCAA |
| | | AAGGATTCGTAGTTAAAGCCAAAAGCAAAAAAATACCCTTAGGGGGTATAC |
| | | CTTCTCCCTCAACTGAGCAGTCTGCGAAAAAGGTAAGAAAGAAAGCAGAGA |
| | | ACGCACATAACACCCCGTTATTAGTTCTTTACGGGTCTAACATGGGAACGG |
| | | CCGAGGGTACAGCCAGGGACCTTGCAGACATTGCTATGTCAAAGGGCTTTG |
| | | CTCCGCAAGTCGCAACCTTAGATTCCCACGCAGGGAACCTGCCGAGAGAAG |
| | | GAGCGGTGCTTATTGTTACCGCTTCATATAATGGTCATCCTCCGGATAACG |
| | | CCAAACAGTTCGTTGACTGGCTGGATCAGGCCAGCGCGGACGAAGTTAAGG |
| | | GGGTTCGTTATAGCGTGTTCGGTTGCGGAGATAAAAATTGGGCCACCACGT |
| | | ATCAGAAAGTACCAGCGTTCATCGACGAAACTTTAGCCGCCAAGGGTGCAG |
| | | AGAACATAGCTGACAGAGGTGAAGCTGACGCCAGTGACGATTTCGAAGGGA |
| | | CATATGAGGAATGGAGAGAGCACATGTGGAGCGACGTCGCCGCTTATTTTA |
| | | ACCTGGATATAGAGAACTCTGAAGACAATAAATCTACTTTGTCTTTGCAGT |
| | | TTGTCGATTCTGCCGCAGACATGCCGCTTGCTAAAATGCACGGCGCCTTCTC |
| | | CACTAATGTCGTGGCGAGCAAGGAATTGCAACAGCCGGGAAGCGCGCGTTC |
| | | CACCCGTCACTTGGAGATTGAGCTGCCCAAGGAGGCTTCATACCAGGAAGG |
| | | CGACCACTTAGGAGTAATACCTAGGAATTATGAAGGTATAGTTAACAGAGT |
| | | CACGGCGCGTTTTGGCCTGGATGCGTCACAGCAGATCCGTCTAGAGGCAGA |
| | | GGAGGAGAAGTTGGCTCATCTTCCCTTGGCGAAGACGGTCAGTGTCGAGGA |
| | | GCTGCTTCAGTATGTCGAGCTGCAAGATCCCGTCACCAGGACGCAACTTCG |
| | | TGCAATGGCCGCAAAGACCGTGTGCCCTCCACACAAAGTGGAACTAGAAGC |
| | | TCTTTTAGAGAAACAAGCGTACAAGGAGCAGGTCCTGGCCAAGCGTTTGAC |
| | | AATGCTTGAACTATTGGAAAAGTACCCAGCGTGTGAAATGAAATTCTCCGA |
| | | ATTTATCGCTCTACTGCCGAGCATCAGACCCCGTTACTACTCTATCTCTTCC |
| | | AGTCCGAGGGTAGATGAAAAGCAGGCATCTATAACAGTCTCCGTCGTCTCT |
| | | GGGGAAGCTTGGAGTGGGTACGGGGAATATAAAGGCATAGCCTCCAATTAC |
| | | CTTGCTGAGCTGCAGGAAGGTGACACAATCACATGTTTCATTTCAACCCCT |
| | | CAAAGTGAATTTACTCTGCCAAAGGATCCGGAGACTCCACTAATCATGGTC |
| | | GGGCCTGGAACTGGTGTAGCGCCTTTTAGAGGTTTCGTCCAAGCCAGAAAG |
| | | CAACTGAAAGAGCAGGGTCAGTCATTAGGCGAAGCCCATTTATACTTTGGA |
| | | TGTAGATCTCCTCATGAAGATTATCTGTACCAGGAAGAACTAGAAAACGCT |
| | | CAAAGCGAAGGGATTATTACACTGCACACGGCCTTCAGCCGTATGCCTAAT |
| | | CAGCCCAAAACCTATGTGCAACATGTAATGGAGCAAGATGGTAAAAAGTTA |
| | | ATTGAGTTGTTAGATCAGGGCGCACATTTTTACATTTGTGGGGATGGCTCT |
| | | CAGATGGCGCCCGCTGTGGAAGCAACCCTGATGAAGAGTTACGCAGATGTA |
| | | CACCAGGTCAGTGAGGCGGATGCAAGGCTGTGGTTACAGCAACTTGAAGAA |
| | | AAGGGCCGTTACGCTAAGGACGTGTGGGCGGGATGA |
| 154 | Bacillus | MTIKEMPQPKTFGELKNLPLLNTDKPVQALMKIADELGEIFKFEAPGRVTRY |
| | megaterium | LSSQRLIKEACDESRFDKNLSQAAKFARDFAGDGLVTSWTHEKNWKKAHNI |
| | species of | LLPSFSQQAMKGYHAMMVDIAVQLVQKWERLNADEHIEVSEDMTRLTLDT |
| | origin; amino | IGLCGFNYRFNSFYRDQPHPFIISMVRAADEVMNKLQRANPDDPAYDENKR |
| | acid sequence; | QFQEDIKVMNDLVDKIIADRKARGEQSDDLLTQMLNGKDPETGEPLDDGNI |
| | 14HC_P450_26 | RYQIITFLIAGHETTSGLLSFALYFLVKNPHVLQKVAEEAARVLVDPVPSYKQ |
| | | VKQLKYVGMVLNEALRLWPTAPAFSLYAKEDTVLGGEYPLEKGDEVMVLIP |
| | | QLHRDKTVWGDDVEEFRPERFENPSAIPQHAFKPFGNGQRACIGQQFALHE |
| | | ATLVLGMMLKHFDFEDHTNYELDIKETLTLKPKGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 155 | Bacillus | ATGACAATAAAGGAAATGCCGCAACCTAAAACTTTCGGCGAATTAAAGAAT |
| | megaterium | CTACCACTACTTAACACAGACAAACCGGTCCAAGCTTTGATGAAGATCGCA |
| | species of | GACGAGCTAGGCGAAATCTTTAAATTTGAGGCACCAGGAAGAGTAACAAGA |
| | origin; nucleic | TACTTGAGTTCTCAAAGACTGATCAAGGAAGCCTGTGATGAATCCAGGTTT |
| | acid sequence; | GATAAAAATCTATCTCAAGCTGCCAAATTCGCTCGTGATTTTGCGGGCGAT |
| | 14HC_P450_26 | GGACTAGTTACAAGTTGGACCCACGAGAAAAACTGGAAGAAGGCGCACAAT |
| | | ATCTTGTTACCCAGTTTCTCCCAACAGGCCATGAAGGGTTATCACGCCATG |
| | | ATGGTTGATATCGCGGTACAGCTGGTTCAAAAATGGGAACGTTTGAATGCA |
| | | GACGAGCATATAGAGGTGAGTGAAGACATGACGCGTCTTACTCTGGATACC |
| | | ATCGGGTTGTGTGGGTTCAACTACAGGTTTAATAGTTTTTACAGGGACCAG |
| | | CCCCATCCATTCATAATTAGTATGGTGAGGGCCGCTGACGAAGTAATGAAC |
| | | AAATTACAACGTGCCAATCCCGACGACCCTGCCTATGACGAGAATAAACGT |
| | | CAGTTCCAAGAGGATATAAAAGTGATGAATGATCTTGTTGATAAGATTAT |
| | | AGCAGATAGAAAGGCGAGAGGAGAACAAAGCGATGATTTGCTGACTCAGA |
| | | TGCTAAACGGAAAGGACCCCGAGACGGGAGAGCCCTTGGACGATGGTAATA |
| | | TTAGATATCAGATCATAACCTTCCTGATCGCCGGACACGAAACAACGAGCG |
| | | GACTTCTTAGCTTCGCCCTATACTTTCTAGTTAAGAACCCTCACGTGCTGCA |
| | | GAAGGTTGCGGAGGAGGCAGCCAGAGTGTTGGTCGATCCTGTACCGTCATA |
| | | CAAACAAGTCAAGCAGCTTAAATACGTTGGAATGGTGCTAAACGAGGCGTT |
| | | GAGGCTTTGGCCTACGGCACCTGCCTTCTCATTGTACGCGAAAGAGGACAC |
| | | GGTACTAGGCGGAGAATATCCTTTGGAGAAGGGCGATGAAGTCATGGTCTT |
| | | AATTCCACAATTACACAGAGACAAAACGGTCTGGGGGGATGACGTTGAAGA |
| | | GTTTAGACCAGAGAGGTTCGAAAATCCATCCGCGATACCTCAGCATGCCTT |
| | | CAAACCGTTTGGCAATGGACAGAGAGCATGCATTGGACAACAATTCGCCCT |
| | | TCATGAGGCTACACTTGTCCTGGGGATGATGTTGAAGCATTTCGACTTTGA |
| | | GGACCATACAAATTACGAGCTAGACATTAAGGAAACGCTAACATTAAAGCC |
| | | GAAGGGCTTCGTCGTAAAAGCTAAGAGTAAGAAGATACCGTTAGGAGGTA |
| | | TCCCTTCCCCTAGTACAGAACAGAGTGCAAAGAAGGTTCGTAAAAAGGCGG |
| | | AGAACGCACATAATACCCCGTTATTAGTTCTTTACGGGTCTAACATGGGAA |
| | | CGGCCGAGGGTACAGCCAGGGACCTTGCAGACATTGCTATGTCAAAGGGCT |
| | | TTGCTCCGCAAGTCGCAACCTTAGATTCCCACGCAGGGAACCTGCCGAGAG |
| | | AAGGAGCGGTGCTTATTGTTACCGCTTCATATAATGGTCATCCTCCGGATA |
| | | ACGCCAAACAGTTCGTTGACTGGCTGGATCAGGCCAGCGCGGACGAAGTTA |
| | | AGGGGGTTCGTTATAGCGTGTTCGGTTGCGGAGATAAAAATTGGGCCACCA |
| | | CGTATCAGAAAGTACCAGCGTTCATCGACGAAACTTTAGCCGCCAAGGGTG |
| | | CAGAGAACATAGCTGACAGAGGTGAAGCTGACGCCAGTGACGATTTCGAAG |
| | | GGACATATGAGGAATGGAGAGAGCACATGTGGAGCGACGTCGCCGCTTATT |
| | | TTAACCTGGATATAGAGAACTCTGAAGACAATAAATCTACTTTGTCTTTGC |
| | | AGTTTGTCGATTCTGCCGCAGACATGCCGCTTGCTAAAATGCACGGCGCCT |
| | | TCTCCACTAATGTCGTGGCGAGCAAGGAATTGCAACAGCCGGGAAGCGCGC |
| | | GTTCCACCCGTCACTTGGAGATTGAGCTGCCCAAGGAGGCTTCATACCAGG |
| | | AAGGCGACCACTTAGGAGTAATACCTAGGAATTATGAAGGTATAGTTAACA |
| | | GAGTCACGGCGCGTTTTGGCCTGGATGCGTCACAGCAGATCCGTCTAGAGG |
| | | CAGAGGAGGAGAAGTTGGCTCATCTTCCCTTGGCGAAGACGGTCAGTGTCG |
| | | AGGAGCTGCTTCAGTATGTCGAGCTGCAAGATCCCGTCACCAGGACGCAAC |
| | | TTCGTGCAATGGCCGCAAAGACCGTGTGCCCTCCACACAAAGTGGAACTAG |
| | | AAGCTCTTTTAGAGAAACAAGCGTACAAGGAGCAGGTCCTGGCCAAGCGTT |
| | | TGACAATGCTTGAACTATTGGAAAAGTACCCAGCGTGTGAAATGAAATTCT |
| | | CCGAATTTATCGCTCTACTGCCGAGCATCAGACCCCGTTACTACTCTATCTC |
| | | TTCCAGTCCGAGGGTAGATGAAAAGCAGGCATCTATAACAGTCTCCGTCGT |
| | | CTCTGGGGAAGCTTGGAGTGGGTACGGGGAATATAAAGGCATAGCCTCCAA |
| | | TTACCTTGCTGAGCTGCAGGAAGGTGACACAATCACATGTTTCATTTCAAC |
| | | CCCTCAAAGTGAATTTACTCTGCCAAAGGATCCGGAGACTCCACTAATCAT |
| | | GGTCGGGCCTGGAACTGGTGTAGCGCCTTTTAGAGGTTTCGTCCAAGCCAG |
| | | AAAGCAACTGAAAGAGCAGGGTCAGTCATTAGGCGAAGCCCATTTATACTT |
| | | TGGATGTAGATCTCCTCATGAAGATTATCTGTACCAGGAAGAACTAGAAAA |
| | | CGCTCAAAGCGAAGGGATTATTACACTGCACACGGCCTTCAGCCGTATGCC |
| | | TAATCAGCCCAAAACCTATGTGCAACATGTAATGGAGCAAGATGGTAAAAA |
| | | GTTAATTGAGTTGTTAGATCAGGGCGCACATTTTTACATTTGTGGGGATGG |
| | | CTCTCAGATGGCGCCCGCTGTGGAAGCAACCCTGATGAAGAGTTACGCAGA |
| | | TGTACACCAGGTCAGTGAGGCGGATGCAAGGCTGTGGTTACAGCAACTTGA |
| | | AGAAAAGGGCCGTTACGCTAAGGACGTGTGGGCGGGATGA |
| 156 | Bacillus | MTIKEMPQPKTFGELKNLPLLNTDKPVQALMKIADELGEIFKFEAPGRVTRY |
| | megaterium | LSSQRLIKEACDESRFDKNLSQALKFARDFAGDGLVTSWTHEKNWKKAHNI |
| | species of | LLPSFSQQAMKGYHAMMVDIAVQLVQKWERLNADEHIEVSEDMTRLTLDT |
| | origin; amino | IGLCGFNYRFNSFYRDQPHPFIISMVRALDEVMNKLQRANPDDPAYDENKR |
| | acid sequence; | QFQEDIKVMNDLVDKIIADRKARGEQSDDLLTQMLNGKDPETGEPLDDGNI |
| | 14HC_P450_27 | RYQIITFLIAGHETTSGLLSFALYFLVKNPHVLQKVAEEAARVLVDPVPSYKQ |
| | | VKQLKYVGMVLNEALRLWPTAPAFSLYAKEDTVLGGEYPLEKGDEVMVLIP |
| | | QLHRDKTVWGDDVEEFRPERFENPSAIPQHAFKPFGNGQRACIGQQFALHE |
| | | ATLVLGMMLKHFDFEDHTNYELDIKETATLKPKGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 157 | Bacillus | ATGACTATAAAGGAGATGCCACAACCAAAAACCTTCGGCGAACTTAAAAAT |
| | megaterium | TTGCCATTATTGAACACTGATAAACCGGTTCAGGCGCTAATGAAAATAGCA |
| | species of | GACGAATTAGGGGAGATCTTTAAATTTGAGGCCCCAGGGAGGGTCACCAGA |
| | origin; nucleic | TATCTTTCCTCTCAACGTTTGATAAAGGAAGCCTGTGACGAGTCCAGATTT |
| | acid sequence; | GATAAAAACTTATCACAAGCTCTGAAGTTCGCCAGAGACTTCGCTGGTGAT |
| | 14HC_P450_27 | GGACTAGTTACATCATGGACTCACGAGAAGAACTGGAAAAAAGCACATAAC |
| | | ATTCTGCTTCCCAGTTTCTCACAACAGGCAATGAAAGGTTACCATGCGATG |
| | | ATGGTGGACATTGCGGTGCAGCTTGTACAAAAATGGGAAAGGTTAAATGCT |
| | | GATGAACATATCGAGGTGAGTGAAGACATGACCAGGCTTACTCTAGACACG |
| | | ATCGGATTATGCGGTTTCAACTACAGATTTAATTCTTTCTACAGAGACCAG |
| | | CCACACCCTTTCATAATCTCTATGGTCCGTGCATTAGATGAAGTGATGAAT |
| | | AAGTTGCAAAGGGCTAATCCCGACGACCCGGCCTACGACGAAAATAAGAGG |
| | | CAATTCCAAGAAGATATTAAAGTGATGAATGATCTTGTGGACAAAATTAT |
| | | TGCAGATCGTAAGGCGAGGGGGGAGCAGTCTGATGACTTACTAACTCAGAT |
| | | GTTGAATGGCAAGGATCCCGAGACCGGAGAACCACTGGACGATGGCAACAT |
| | | CCGTTATCAGATTATAACTTTCTTAATCGCTGGCCACGAGACCACATCTGG |
| | | ACTTCTGTCATTTGCACTATACTTCCTAGTTAAGAATCCGCACGTGCTGCA |
| | | GAAGGTGGCGGAAGAGGCTGCTCGTGTCCTAGTCGACCCCGTACCCAGCTA |
| | | TAAACAGGTAAAACAGCTTAAGTATGTTGGGATGGTCCTGAATGAGGCGCT |
| | | GAGACTATGGCCAACTGCGCCAGCCTTTAGCCTTTATGCCAAAGAGGATAC |
| | | CGTCTTAGGAGGTGAGTATCCACTGGAGAAAGGAGATGAAGTTATGGTGCT |
| | | TATCCCTCAGTTGCATCGTGATAAGACCGTGTGGGGTGATGATGTGGAAGA |
| | | ATTTAGGCCCGAGAGATTTGAGAATCCTTCCGCGATTCCCCAGCATGCTTT |
| | | CAAGCCCTTTGGCAACGGACAGAGAGCTTGCATTGGGCAACAGTTTGCTTT |
| | | GCATGAGGCAACGCTAGTTTTGGGGATGATGCTAAAGCATTTCGATTTCGA |
| | | AGACCATACTAACTATGAACTTGATATCAAGGAGACAGCTACCCTAAAACC |
| | | CAAGGGTTTCGTGGTTAAGGCGAAGTCTAAAAAAATACCACTAGGGGGGAT |
| | | CCCATCACCGTCAACTGAGCAGTCCGCCAAAAAAGTCCGTAAGAAAGCCGA |
| | | AAATGCGCATAATACCCCGTTATTAGTTCTTTACGGGTCTAACATGGGAAC |
| | | GGCCGAGGGTACAGCCAGGGACCTTGCAGACATTGCTATGTCAAAGGGCTT |
| | | TGCTCCGCAAGTCGCAACCTTAGATTCCCACGCAGGGAACCTGCCGAGAGA |
| | | AGGAGCGGTGCTTATTGTTACCGCTTCATATAATGGTCATCCTCCGGATAA |
| | | CGCCAAACAGTTCGTTGACTGGCTGGATCAGGCCAGCGCGGACGAAGTTAA |
| | | GGGGGTTCGTTATAGCGTGTTCGGTTGCGGAGATAAAAATTGGGCCACCAC |
| | | GTATCAGAAAGTACCAGCGTTCATCGACGAAACTTTAGCCGCCAAGGGTGC |
| | | AGAGAACATAGCTGACAGAGGTGAAGCTGACGCCAGTGACGATTTCGAAGG |
| | | GACATATGAGGAATGGAGAGAGCACATGTGGAGCGACGTCGCCGCTTATTT |
| | | TAACCTGGATATAGAGAACTCTGAAGACAATAAATCTACTTTGTCTTTGCA |
| | | GTTTGTCGATTCTGCCGCAGACATGCCGCTTGCTAAAATGCACGGCGCCTTC |
| | | TCCACTAATGTCGTGGCGAGCAAGGAATTGCAACAGCCGGGAAGCGCGCGT |
| | | TCCACCCGTCACTTGGAGATTGAGCTGCCCAAGGAGGCTTCATACCAGGAA |
| | | GGCGACCACTTAGGAGTAATACCTAGGAATTATGAAGGTATAGTTAACAGA |
| | | GTCACGGCGCGTTTTGGCCTGGATGCGTCACAGCAGATCCGTCTAGAGGCA |
| | | GAGGAGGAGAAGTTGGCTCATCTTCCCTTGGCGAAGACGGTCAGTGTCGAG |
| | | GAGCTGCTTCAGTATGTCGAGCTGCAAGATCCCGTCACCAGGACGCAACTT |
| | | CGTGCAATGGCCGCAAAGACCGTGTGCCCTCCACACAAAGTGGAACTAGAA |
| | | GCTCTTTTAGAGAAACAAGCGTACAAGGAGCAGGTCCTGGCCAAGCGTTTG |
| | | ACAATGCTTGAACTATTGGAAAAGTACCCAGCGTGTGAAATGAAATTCTCC |
| | | GAATTTATCGCTCTACTGCCGAGCATCAGACCCCGTTACTACTCTATCTCTT |
| | | CCAGTCCGAGGGTAGATGAAAAGCAGGCATCTATAACAGTCTCCGTCGTCT |
| | | CTGGGGAAGCTTGGAGTGGGTACGGGGAATATAAAGGCATAGCCTCCAATT |
| | | ACCTTGCTGAGCTGCAGGAAGGTGACACAATCACATGTTTCATTTCAACCC |
| | | CTCAAAGTGAATTTACTCTGCCAAAGGATCCGGAGACTCCACTAATCATGG |
| | | TCGGGCCTGGAACTGGTGTAGCGCCTTTTAGAGGTTTCGTCCAAGCCAGAA |
| | | AGCAACTGAAAGAGCAGGGTCAGTCATTAGGCGAAGCCCATTTATACTTTG |
| | | GATGTAGATCTCCTCATGAAGATTATCTGTACCAGGAAGAACTAGAAAACG |
| | | CTCAAAGCGAAGGGATTATTACACTGCACACGGCCTTCAGCCGTATGCCTA |
| | | ATCAGCCCAAAACCTATGTGCAACATGTAATGGAGCAAGATGGTAAAAAGT |
| | | TAATTGAGTTGTTAGATCAGGGCGCACATTTTTACATTTGTGGGGATGGCT |
| | | CTCAGATGGCGCCCGCTGTGGAAGCAACCCTGATGAAGAGTTACGCAGATG |
| | | TACACCAGGTCAGTGAGGCGGATGCAAGGCTGTGGTTACAGCAACTTGAAG |
| | | AAAAGGGCCGTTACGCTAAGGACGTGTGGGCGGGATGA |
| 158 | Bacillus | MTIKEMPQPKTFGELKNLPLLNTDKPVQALMKIADELGEIFKFEAPGLVTRY |
| | megaterium | LSSQRLIKEACDESRFDKNLSQALKFVRDFAGDGIVTSWTHEKNWKKAHNI |
| | species of | LLPSFSQQAMKGYHAMMVDIAVQLVQKWERLNADEHIEVPEDMTRLTLDT |
| | origin; amino | IGLCGFNYRFNSFYRDQPHPFITSMVRALDEAMNKQQRANPDDPAYDENKR |
| | acid sequence; | QFQEDIKVMNDLVDKIIADRKASGEQSDDLLTHMLNGKDPETGEPLDDENI |
| | 14HC_P450_28 | RYQIITFLIAGHETTSGLLSFALYFLVKNPHVLQKAAEEAARVLVDPVPSYKQ |
| | | VKQLKYVGMVLTEALRLWPTAPAFSLYAKEDTVLGGEYPLEKGDELMVLIP |
| | | QLHRDKTIWGDDVEEFRPERFENPSAIPQHAFKPFGNGQRACIGQQFALHE |
| | | ATLVLGMMLKHFDFEDHTNYELDIKETLTLKPEGFVVKAKSKKIPLGGIPSP |
| | | STEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 159 | Bacillus | ATGACAATCAAAGAAATGCCCCAGCCGAAAACCTTCGGTGAGCTAAAAAAT |
| | megaterium | TTGCCCTTATTAAACACAGATAAGCCGGTACAAGCTCTTATGAAAATAGCC |
| | species of | GATGAACTGGGGGAAATTTTTAAGTTTGAAGCTCCTGGACTAGTAACTAGA |
| | origin; nucleic | TATTTGTCTTCTCAAAGACTAATCAAAGAGGCATGCGATGAGTCCAGATTC |
| | acid sequence; | GATAAAAATTTAAGTCAGGCTCTTAAATTCGTTAGAGACTTTGCCGGTGAC |
| | 14HC_P450_28 | GGAATTGTTACAAGCTGGACACATGAAAAAAACTGGAAAAAGGCCCATAAC |
| | | ATTTTACTGCCGTCCTTTTCTCAACAGGCAATGAAAGGGTACCACGCAATG |
| | | ATGGTCGATATTGCCGTTCAGTTAGTACAGAAATGGGAGAGACTAAATGCG |
| | | GATGAACATATAGAGGTGCCAGAAGATATGACGAGGTTAACATTGGACACC |
| | | ATTGGTCTGTGCGGATTTAATTACCGTTTCAACAGTTTCTACCGTGATCAG |
| | | CCCCATCCATTTATAACCAGCATGGTCAGAGCGCTTGATGAAGCTATGAAT |
| | | AAACAACAAAGAGCCAATCCTGACGACCCAGCATATGATGAGAACAAAAGA |
| | | CAGTTCCAGGAAGATATAAAAGTGATGAATGATTTGGTAGACAAGATCAT |
| | | AGCCGACAGAAAGGCTTCAGGCGAACAGAGTGACGATTTATTAACTCATAT |
| | | GCTAAATGGTAAGGACCCGGAAACAGGCGAGCCTCTTGATGATGAGAACAT |
| | | AAGATATCAAATAATCACATTTTTGATCGCGGGCCATGAGACGACGAGTGG |
| | | CTTGTTAAGTTTCGCGCTTTATTTTTTAGTAAAAAACCCTCATGTGCTTCA |
| | | GAAAGCGGCTGAAGAGGCGGCTCGTGTTTTGGTAGACCCAGTGCCTAGCTA |
| | | CAAACAAGTTAAGCAACTTAAATATGTCGGGATGGTATTAACCGAGGCGCT |
| | | GCGTTTGTGGCCAACAGCCCCTGCGTTCTCCCTATACGCAAAGGAGGACAC |
| | | AGTGCTAGGCGGCGAGTATCCATTAGAAAAGGGAGATGAGCTAATGGTTTT |
| | | GATCCCTCAACTTCACCGTGACAAAACGATTTGGGGCGATGATGTGGAGGA |
| | | GTTCAGGCCAGAAAGGTTCGAGAACCCAAGTGCTATCCCCCAGCACGCATT |
| | | TAAGCCCTTTGGGAACGGCCAGCGTGCCTGTATTGGCCAACAGTTCGCACT |
| | | GCACGAGGCGACGCTTGTCCTTGGGATGATGCTAAAACATTTTGATTTTGA |
| | | GGACCATACAAATTACGAGTTAGATATAAAGGAAACTCTGACTCTAAAACC |
| | | GGAAGGTTTTGTCGTTAAAGCCAAATCAAAGAAAATACCTCTTGGTGGAAT |
| | | ACCGTCCCCGAGCACTGAGCAGTCCGCGAAAAAAGTACGTAAGAAGGCGGA |
| | | AAATGCTCACAATACCCCGTTATTAGTTCTTTACGGGTCTAACATGGGAAC |
| | | GGCCGAGGGTACAGCCAGGGACCTTGCAGACATTGCTATGTCAAAGGGCTT |
| | | TGCTCCGCAAGTCGCAACCTTAGATTCCCACGCAGGGAACCTGCCGAGAGA |
| | | AGGAGCGGTGCTTATTGTTACCGCTTCATATAATGGTCATCCTCCGGATAA |
| | | CGCCAAACAGTTCGTTGACTGGCTGGATCAGGCCAGCGCGGACGAAGTTAA |
| | | GGGGGTTCGTTATAGCGTGTTCGGTTGCGGAGATAAAAATTGGGCCACCAC |
| | | GTATCAGAAAGTACCAGCGTTCATCGACGAAACTTTAGCCGCCAAGGGTGC |
| | | AGAGAACATAGCTGACAGAGGTGAAGCTGACGCCAGTGACGATTTCGAAGG |
| | | GACATATGAGGAATGGAGAGAGCACATGTGGAGCGACGTCGCCGCTTATTT |
| | | TAACCTGGATATAGAGAACTCTGAAGACAATAAATCTACTTTGTCTTTGCA |
| | | GTTTGTCGATTCTGCCGCAGACATGCCGCTTGCTAAAATGCACGGCGCCTTC |
| | | TCCACTAATGTCGTGGCGAGCAAGGAATTGCAACAGCCGGGAAGCGCGCGT |
| | | TCCACCCGTCACTTGGAGATTGAGCTGCCCAAGGAGGCTTCATACCAGGAA |
| | | GGCGACCACTTAGGAGTAATACCTAGGAATTATGAAGGTATAGTTAACAGA |
| | | GTCACGGCGCGTTTTGGCCTGGATGCGTCACAGCAGATCCGTCTAGAGGCA |
| | | GAGGAGGAGAAGTTGGCTCATCTTCCCTTGGCGAAGACGGTCAGTGTCGAG |
| | | GAGCTGCTTCAGTATGTCGAGCTGCAAGATCCCGTCACCAGGACGCAACTT |
| | | CGTGCAATGGCCGCAAAGACCGTGTGCCCTCCACACAAAGTGGAACTAGAA |
| | | GCTCTTTTAGAGAAACAAGCGTACAAGGAGCAGGTCCTGGCCAAGCGTTTG |
| | | ACAATGCTTGAACTATTGGAAAAGTACCCAGCGTGTGAAATGAAATTCTCC |
| | | GAATTTATCGCTCTACTGCCGAGCATCAGACCCCGTTACTACTCTATCTCTT |
| | | CCAGTCCGAGGGTAGATGAAAAGCAGGCATCTATAACAGTCTCCGTCGTCT |
| | | CTGGGGAAGCTTGGAGTGGGTACGGGGAATATAAAGGCATAGCCTCCAATT |
| | | ACCTTGCTGAGCTGCAGGAAGGTGACACAATCACATGTTTCATTTCAACCC |
| | | CTCAAAGTGAATTTACTCTGCCAAAGGATCCGGAGACTCCACTAATCATGG |
| | | TCGGGCCTGGAACTGGTGTAGCGCCTTTTAGAGGTTTCGTCCAAGCCAGAA |
| | | AGCAACTGAAAGAGCAGGGTCAGTCATTAGGCGAAGCCCATTTATACTTTG |
| | | GATGTAGATCTCCTCATGAAGATTATCTGTACCAGGAAGAACTAGAAAACG |
| | | CTCAAAGCGAAGGGATTATTACACTGCACACGGCCTTCAGCCGTATGCCTA |
| | | ATCAGCCCAAAACCTATGTGCAACATGTAATGGAGCAAGATGGTAAAAAGT |
| | | TAATTGAGTTGTTAGATCAGGGCGCACATTTTTACATTTGTGGGGATGGCT |
| | | CTCAGATGGCGCCCGCTGTGGAAGCAACCCTGATGAAGAGTTACGCAGATG |
| | | TACACCAGGTCAGTGAGGCGGATGCAAGGCTGTGGTTACAGCAACTTGAAG |
| | | AAAAGGGCCGTTACGCTAAGGACGTGTGGGCGGGATGA |
| 160 | Bacillus | MTIKEMPQPKTFGELKNLPLLNTDKPVQALMKIADELGEIFKFEAPGLVTRY |
| | megaterium | LSSQRLIKEACDESRFDKNLSQALKFVRDIAGDGLVTSWTHEKNWKKAHNI |
| | species of | LLPSFSQQAMKGYHAMMVDIAVQLVQKWERLNADEHIEVPEDMTRLTLDT |
| | origin; amino | IGLCGFNYRFNSFYRDQPHPFITSMVRALDEAMNKQQRANPDDPAYDENKR |
| | acid sequence; | QFQEDIKVMNDLVDKIIADRKASGEQSDDLLTHMLNGKDPETGEPLDDENI |
| | 14HC_P450_29 | RYQIITFLIAGHVTTSGLLSFALYFLVKNPHVLQKAAEEAARVLVDPVPSYKQ |
| | | VKQLKYVGMVLNEALRLWPTAPAFSLYAKEDTVLGGEYPLEKGDELMVLIP |
| | | QLHRDKTIWGDDVEEFRPERFENPSAIPQHAFKPFGNGQRACIGQQFALHE |
| | | ATLVLSMMLKHFDFEDHTNYELDIKETLTLKPEGFVVKAKSKKIPLGGIPSPS |
| | | TEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 161 | Bacillus | ATGACAATTAAAGAGATGCCGCAACCGAAAACTTTTGGTGAACTAAAAAAC |
| | megaterium | TTGCCTTTGCTAAACACGGATAAACCTGTGCAGGCCTTAATGAAAATTGCA |
| | species of | GATGAGCTAGGTGAAATTTTTAAATTCGAAGCCCCCGGCTTAGTGACGAGG |
| | origin; nucleic | TATCTATCATCCCAGAGGCTGATTAAGGAGGCTTGCGACGAATCCAGATTC |
| | acid sequence; | GATAAGAACCTAAGCCAAGCGCTGAAGTTCGTAAGGGACATCGCTGGCGAC |
| | 14HC_P450_29 | GGGCTGGTGACTTCCTGGACCCATGAAAAAAATTGGAAAAAGGCTCATAAC |
| | | ATCTTGTTACCATCATTTTCCCAACAGGCGATGAAGGGTTATCACGCTATG |
| | | ATGGTAGATATCGCCGTCCAGTTAGTCCAAAAGTGGGAACGTCTTAACGCC |
| | | GACGAGCATATCGAGGTACCTGAGGACATGACAAGGCTAACGTTAGACACG |
| | | ATCGGATTGTGTGGTTTCAATTACCGTTTCAACTCATTCTATAGGGACCAA |
| | | CCTCATCCCTTTATAACGAGCATGGTTCGTGCCCTGGATGAAGCAATGAAC |
| | | AAACAACAGAGGGCCAATCCGGACGACCCGGCATATGATGAGAACAAGAGA |
| | | CAGTTTCAAGAGGATATAAAAGTTATGAATGACCTTGTAGACAAGATCATC |
| | | GCGGATCGTAAGGCGTCTGGGGAACAGTCTGACGACCTTTTAACGCATATG |
| | | CTTAATGGAAAGGACCCGGAGACAGGAGAACCATTGGATGATGAGAACATT |
| | | AGGTATCAGATAATCACTTTTCTTATAGCAGGCCATGTCACGACGTCCGGG |
| | | CTGTTATCCTTTGCTCTATATTTTCTAGTGAAGAACCCTCACGTATTGCAG |
| | | AAGGCAGCCGAGGAAGCTGCTAGAGTCCTAGTTGATCCGGTCCCTAGTTAT |
| | | AAACAAGTTAAACAGTTGAAATATGTTGGTATGGTGTTAAATGAGGCACT |
| | | GAGACTATGGCCTACGGCACCAGCTTTCAGCTTGTATGCCAAGGAAGATAC |
| | | GGTCTTAGGGGGAGAATATCCACTAGAGAAGGGCGATGAATTGATGGTGTT |
| | | GATTCCGCAATTGCACAGAGACAAAACGATTTGGGGGGACGATGTTGAGGA |
| | | ATTCCGTCCTGAAAGATTTGAGAACCCATCAGCCATCCCCCAGCACGCATTC |
| | | AAGCCCTTTGGGAACGGGCAGAGGGCATGTATCGGCCAACAGTTCGCACTG |
| | | CATGAGGCAACCCTGGTACTATCAATGATGTTAAAACATTTCGACTTCGAA |
| | | GACCACACCAATTATGAGTTAGACATCAAGGAGACTCTGACCCTGAAACCG |
| | | GAAGGATTCGTGGTAAAGGCCAAGTCAAAGAAGATTCCACTAGGTGGTATT |
| | | CCGAGCCCGTCCACTGAGCAAAGTGCTAAGAAGGTTCGTAAAAAAGCGGAA |
| | | AATGCACACAATACCCCGTTATTAGTTCTTTACGGGTCTAACATGGGAACG |
| | | GCCGAGGGTACAGCCAGGGACCTTGCAGACATTGCTATGTCAAAGGGCTTT |
| | | GCTCCGCAAGTCGCAACCTTAGATTCCCACGCAGGGAACCTGCCGAGAGAA |
| | | GGAGCGGTGCTTATTGTTACCGCTTCATATAATGGTCATCCTCCGGATAAC |
| | | GCCAAACAGTTCGTTGACTGGCTGGATCAGGCCAGCGCGGACGAAGTTAAG |
| | | GGGGTTCGTTATAGCGTGTTCGGTTGCGGAGATAAAAATTGGGCCACCACG |
| | | TATCAGAAAGTACCAGCGTTCATCGACGAAACTTTAGCCGCCAAGGGTGCA |
| | | GAGAACATAGCTGACAGAGGTGAAGCTGACGCCAGTGACGATTTCGAAGGG |
| | | ACATATGAGGAATGGAGAGAGCACATGTGGAGCGACGTCGCCGCTTATTTT |
| | | AACCTGGATATAGAGAACTCTGAAGACAATAAATCTACTTTGTCTTTGCAG |
| | | TTTGTCGATTCTGCCGCAGACATGCCGCTTGCTAAAATGCACGGCGCCTTCT |
| | | CCACTAATGTCGTGGCGAGCAAGGAATTGCAACAGCCGGGAAGCGCGCGTT |
| | | CCACCCGTCACTTGGAGATTGAGCTGCCCAAGGAGGCTTCATACCAGGAAG |
| | | GCGACCACTTAGGAGTAATACCTAGGAATTATGAAGGTATAGTTAACAGAG |
| | | TCACGGCGCGTTTTGGCCTGGATGCGTCACAGCAGATCCGTCTAGAGGCAG |
| | | AGGAGGAGAAGTTGGCTCATCTTCCCTTGGCGAAGACGGTCAGTGTCGAGG |
| | | AGCTGCTTCAGTATGTCGAGCTGCAAGATCCCGTCACCAGGACGCAACTTC |
| | | GTGCAATGGCCGCAAAGACCGTGTGCCCTCCACACAAAGTGGAACTAGAAG |
| | | CTCTTTTAGAGAAACAAGCGTACAAGGAGCAGGTCCTGGCCAAGCGTTTGA |
| | | CAATGCTTGAACTATTGGAAAAGTACCCAGCGTGTGAAATGAAATTCTCCG |
| | | AATTTATCGCTCTACTGCCGAGCATCAGACCCCGTTACTACTCTATCTCTTC |
| | | CAGTCCGAGGGTAGATGAAAAGCAGGCATCTATAACAGTCTCCGTCGTCTC |
| | | TGGGGAAGCTTGGAGTGGGTACGGGGAATATAAAGGCATAGCCTCCAATTA |
| | | CCTTGCTGAGCTGCAGGAAGGTGACACAATCACATGTTTCATTTCAACCCC |
| | | TCAAAGTGAATTTACTCTGCCAAAGGATCCGGAGACTCCACTAATCATGGT |
| | | CGGGCCTGGAACTGGTGTAGCGCCTTTTAGAGGTTTCGTCCAAGCCAGAAA |
| | | GCAACTGAAAGAGCAGGGTCAGTCATTAGGCGAAGCCCATTTATACTTTGG |
| | | ATGTAGATCTCCTCATGAAGATTATCTGTACCAGGAAGAACTAGAAAACGC |
| | | TCAAAGCGAAGGGATTATTACACTGCACACGGCCTTCAGCCGTATGCCTAA |
| | | TCAGCCCAAAACCTATGTGCAACATGTAATGGAGCAAGATGGTAAAAAGTT |
| | | AATTGAGTTGTTAGATCAGGGCGCACATTTTTACATTTGTGGGGATGGCTC |
| | | TCAGATGGCGCCCGCTGTGGAAGCAACCCTGATGAAGAGTTACGCAGATGT |
| | | ACACCAGGTCAGTGAGGCGGATGCAAGGCTGTGGTTACAGCAACTTGAAGA |
| | | AAAGGGCCGTTACGCTAAGGACGTGTGGGCGGGATGA |
| 162 | Bacillus | MTIKEMPQPKTFGELKNLPLLNTDKPVQALMKIADELGEIFKFEAPGLVTRY |
| | megaterium | LSSQRLIKEACDGSRFDKNLSQALKFVRDIAGDGLVTSWTHEKNWKKAHNI |
| | species of | LLPSFSQQAMKGYHAMMVDIAVQLVQKWERLNADEHIEVPGDMTRLTLDT |
| | origin; amino | IGLCGFNYRFNSFYRDQPHPFITSMVRALDEAMNKQQRANPDDPAYDENKR |
| | acid sequence; | QFQEDIKVMNDLVDKIIADRKASGEQSDDLLTHMLNGKDPETGEPLDDENI |
| | 14HC_P450_30 | RYQIITFLIAGHVTTSGLLSFALYFLVKNPHVLQKAAEEAARVLVDPVPSYKQ |
| | | VKQLKYVGMVLNEALRLWPTAPAFSLYAKEDTVLGGEYPLEKGDELMVLIP |
| | | QLHRDKTIWGDDVEEFRPERFENPSAIPQHAFKPFGNGQRACIGQQFALHE |
| | | ATLVLSMMLKHFDFEDHTNYELDIKETLTLKPEGFVVKAKSKKIPLGGIPSPS |
| | | TEQSAKKVRKKAENAHNTPLLVLYGSNMGTAEGTARDLADIAMSKGFAPQ |
| | | VATLDSHAGNLPREGAVLIVTASYNGHPPDNAKQFVDWLDQASADEVKGV |
| | | RYSVFGCGDKNWATTYQKVPAFIDETLAAKGAENIADRGEADASDDFEGTY |
| | | EEWREHMWSDVAAYFNLDIENSEDNKSTLSLQFVDSAADMPLAKMHGAFS |
| | | TNVVASKELQQPGSARSTRHLEIELPKEASYQEGDHLGVIPRNYEGIVNRVT |
| | | ARFGLDASQQIRLEAEEEKLAHLPLAKTVSVEELLQYVELQDPVTRTQLRAM |
| | | AAKTVCPPHKVELEALLEKQAYKEQVLAKRLTMLELLEKYPACEMKFSEFIA |
| | | LLPSIRPRYYSISSSPRVDEKQASITVSVVSGEAWSGYGEYKGIASNYLAELQE |
| | | GDTITCFISTPQSEFTLPKDPETPLIMVGPGTGVAPFRGFVQARKQLKEQGQS |
| | | LGEAHLYFGCRSPHEDYLYQEELENAQSEGIITLHTAFSRMPNQPKTYVQHV |
| | | MEQDGKKLIELLDQGAHFYICGDGSQMAPAVEATLMKSYADVHQVSEADAR |
| | | LWLQQLEEKGRYAKDVWAG* |
| 163 | Bacillus | ATGACAATAAAGGAGATGCCCCAGCCCAAAACATTCGGGGAATTAAAAAAT |
| | megaterium | CTACCACTTCTTAACACCGACAAACCTGTTCAAGCGCTGATGAAAATTGCG |
| | species of | GACGAACTTGGGGAGATTTTCAAGTTCGAAGCACCGGGTCTTGTGACCCGT |
| | origin; nucleic | TATTTATCTTCCCAGAGGCTTATTAAGGAGGCTTGTGACGGCTCAAGGTTT |
| | acid sequence; | GATAAAAATTTGAGCCAAGCTCTGAAATTTGTCAGAGATATAGCCGGTGAC |
| | 14HC_P450_30 | GGCCTGGTCACAAGCTGGACTCACGAAAAAAATTGGAAGAAGGCTCATAAT |
| | | ATACTATTGCCCTCATTTAGCCAGCAGGCTATGAAAGGATATCACGCGATG |
| | | ATGGTCGATATCGCAGTGCAACTAGTGCAGAAGTGGGAACGTTTGAACGCG |
| | | GATGAACATATTGAGGTACCTGGGGATATGACCAGGCTAACTCTTGACACG |
| | | ATAGGGTTGTGTGGTTTTAACTATAGGTTCAATTCATTTTATAGGGATCAA |
| | | CCACACCCGTTCATAACCAGCATGGTGCGTGCGTTGGATGAAGCGATGAAC |
| | | AAACAACAGAGGGCGAACCCAGATGATCCAGCTTACGATGAAAATAAAAGA |
| | | CAATTTCAGGAAGATATTAAAGTAATGAACGATTTGGTTGATAAAATAAT |
| | | AGCGGATAGGAAGGCTAGCGGCGAGCAATCCGACGATCTTCTTACCCACAT |
| | | GTTAAACGGCAAAGATCCGGAGACGGGGGAGCCATTGGATGATGAAAACAT |
| | | AAGATACCAGATTATCACATTTCTTATCGCGGGACATGTCACTACATCCGG |
| | | GCTTCTAAGCTTTGCACTATACTTCCTAGTGAAGAATCCTCATGTACTACA |
| | | AAAAGCGGCCGAAGAAGCAGCACGTGTACTGGTTGACCCTGTGCCCAGTTA |
| | | TAAACAAGTGAAACAGTTAAAATATGTCGGAATGGTTCTAAATGAGGCGT |
| | | TACGTCTATGGCCGACAGCACCCGCATTCTCCCTTTATGCTAAAGAAGACA |
| | | CTGTTTTAGGAGGCGAATACCCCTTGGAAAAGGGCGACGAACTAATGGTGT |
| | | TGATCCCACAATTACACAGAGATAAAACGATCTGGGGGGATGATGTAGAAG |
| | | AATTTAGGCCTGAGAGGTTCGAAAACCCATCCGCGATACCCCAGCATGCTT |
| | | TCAAACCGTTCGGGAATGGTCAAAGGGCATGCATTGGTCAGCAGTTTGCAC |
| | | TGCATGAGGCCACCCTAGTCCTGAGTATGATGCTAAAGCATTTCGATTTCG |
| | | AAGATCACACCAACTATGAGTTAGATATCAAGGAGACCCTAACGCTTAAAC |
| | | CGGAGGGCTTCGTTGTAAAGGCGAAATCCAAAAAGATCCCATTGGGTGGAA |
| | | TTCCCTCACCCTCAACAGAACAATCCGCCAAAAAAGTTAGAAAGAAGGCAG |
| | | AAAATGCCCATAATACCCCGTTATTAGTTCTTTACGGGTCTAACATGGGAA |
| | | CGGCCGAGGGTACAGCCAGGGACCTTGCAGACATTGCTATGTCAAAGGGCT |
| | | TTGCTCCGCAAGTCGCAACCTTAGATTCCCACGCAGGGAACCTGCCGAGAG |
| | | AAGGAGCGGTGCTTATTGTTACCGCTTCATATAATGGTCATCCTCCGGATA |
| | | ACGCCAAACAGTTCGTTGACTGGCTGGATCAGGCCAGCGCGGACGAAGTTA |
| | | AGGGGGTTCGTTATAGCGTGTTCGGTTGCGGAGATAAAAATTGGGCCACCA |
| | | CGTATCAGAAAGTACCAGCGTTCATCGACGAAACTTTAGCCGCCAAGGGTG |
| | | CAGAGAACATAGCTGACAGAGGTGAAGCTGACGCCAGTGACGATTTCGAAG |
| | | GGACATATGAGGAATGGAGAGAGCACATGTGGAGCGACGTCGCCGCTTATT |
| | | TTAACCTGGATATAGAGAACTCTGAAGACAATAAATCTACTTTGTCTTTGC |
| | | AGTTTGTCGATTCTGCCGCAGACATGCCGCTTGCTAAAATGCACGGCGCCT |
| | | TCTCCACTAATGTCGTGGCGAGCAAGGAATTGCAACAGCCGGGAAGCGCGC |
| | | GTTCCACCCGTCACTTGGAGATTGAGCTGCCCAAGGAGGCTTCATACCAGG |
| | | AAGGCGACCACTTAGGAGTAATACCTAGGAATTATGAAGGTATAGTTAACA |
| | | GAGTCACGGCGCGTTTTGGCCTGGATGCGTCACAGCAGATCCGTCTAGAGG |
| | | CAGAGGAGGAGAAGTTGGCTCATCTTCCCTTGGCGAAGACGGTCAGTGTCG |
| | | AGGAGCTGCTTCAGTATGTCGAGCTGCAAGATCCCGTCACCAGGACGCAAC |
| | | TTCGTGCAATGGCCGCAAAGACCGTGTGCCCTCCACACAAAGTGGAACTAG |
| | | AAGCTCTTTTAGAGAAACAAGCGTACAAGGAGCAGGTCCTGGCCAAGCGTT |
| | | TGACAATGCTTGAACTATTGGAAAAGTACCCAGCGTGTGAAATGAAATTCT |
| | | CCGAATTTATCGCTCTACTGCCGAGCATCAGACCCCGTTACTACTCTATCTC |
| | | TTCCAGTCCGAGGGTAGATGAAAAGCAGGCATCTATAACAGTCTCCGTCGT |
| | | CTCTGGGGAAGCTTGGAGTGGGTACGGGGAATATAAAGGCATAGCCTCCAA |
| | | TTACCTTGCTGAGCTGCAGGAAGGTGACACAATCACATGTTTCATTTCAAC |
| | | CCCTCAAAGTGAATTTACTCTGCCAAAGGATCCGGAGACTCCACTAATCAT |
| | | GGTCGGGCCTGGAACTGGTGTAGCGCCTTTTAGAGGTTTCGTCCAAGCCAG |
| | | AAAGCAACTGAAAGAGCAGGGTCAGTCATTAGGCGAAGCCCATTTATACTT |
| | | TGGATGTAGATCTCCTCATGAAGATTATCTGTACCAGGAAGAACTAGAAAA |
| | | CGCTCAAAGCGAAGGGATTATTACACTGCACACGGCCTTCAGCCGTATGCC |
| | | TAATCAGCCCAAAACCTATGTGCAACATGTAATGGAGCAAGATGGTAAAAA |
| | | GTTAATTGAGTTGTTAGATCAGGGCGCACATTTTTACATTTGTGGGGATGG |
| | | CTCTCAGATGGCGCCCGCTGTGGAAGCAACCCTGATGAAGAGTTACGCAGA |
| | | TGTACACCAGGTCAGTGAGGCGGATGCAAGGCTGTGGTTACAGCAACTTGA |
| | | AGAAAAGGGCCGTTACGCTAAGGACGTGTGGGCGGGATGA |
| 164 | Rattus | MEPTILLLLALLVGFLLLLVRGHPKSRGNFPPGPRPLPLLGNLLQLDRGGLLN |
| | norvegicus | SFMQLREKYGDVFTVHLGPRPVVMLCGTDTIKEALVGQAEDFSGRGTIAVIE |
| | species of | PIFKEYGVIFANGERWKALRRFSLATMRDFGMGKRSVEERIQEEAQCLVEEL |
| | origin; amino | RKSQGAPLDPTFLFQCITANIICSIVFGERFDYTDRQFLRLLELFYRTFSLLSSF |
| | acid sequence; | SSQVFEFFSGFLKYFPGAHRQISKNLQEILDYIGHIVEKHRATLDPSAPRDFID |
| | 14HC_P450_31 | TYLLRMEKEKSNHHTEFHHENLMISLLSLFFAGTETSSTTLRYGFLLMLKYP |
| | | HVAEKVQKEIDQVIGSHRLPTLDDRSKMPYTDAVIHEIQRFSDLVPIGVPHR |
| | | VTKDTMFRGYLLPKNTEVYPILSSALHDPQYFDHPDSFNPEHFLDANGALK |
| | | KSEAFMPFSTGKRICLGEGIARNELFLFFTTILQNFSVSSHLAPKDIDLTPKES |
| | | GIGKIPPTYQICFSAR* |
| 165 | Rattus | ATGGAGCCCACCATATTGCTACTGTTGGCGTTATTAGTCGGATTTTTGCTA |
| | norvegicus | CTGCTAGTGAGAGGACATCCAAAGTCCCGTGGCAACTTCCCACCCGGTCCA |
| | species of | AGGCCGTTACCCCTTTTGGGTAATTTATTACAACTTGATAGGGGAGGACTA |
| | origin; nucleic | TTGAATAGTTTCATGCAACTAAGAGAGAAATACGGTGATGTGTTCACAGTT |
| | acid sequence; | CACTTGGGGCCGAGACCTGTAGTAATGCTGTGTGGGACTGATACTATAAAA |
| | 14HC_P450_31 | GAGGCCTTAGTCGGGCAAGCGGAAGACTTCTCAGGAAGAGGCACCATAGCA |
| | | GTAATTGAACCTATCTTCAAAGAATACGGAGTCATATTCGCTAACGGCGAG |
| | | CGTTGGAAGGCGCTGCGTAGATTCTCACTTGCTACTATGAGGGATTTTGGC |
| | | ATGGGGAAGCGTAGCGTGGAAGAGAGAATCCAAGAGGAGGCACAGTGCTT |
| | | AGTAGAGGAACTACGTAAGTCCCAGGGCGCTCCGTTGGACCCAACGTTTTT |
| | | GTTCCAGTGCATCACGGCTAACATCATCTGCTCCATAGTCTTTGGAGAACG |
| | | TTTCGACTACACTGACAGGCAATTTTTACGTTTACTGGAACTGTTTTACCG |
| | | TACGTTTTCACTACTTTCAAGTTTCAGTAGTCAGGTCTTTGAGTTTTTCTC |
| | | TGGATTCTTGAAGTACTTTCCAGGGGCCCATAGGCAGATCAGTAAAAACCT |
| | | ACAAGAAATTTTAGACTACATTGGACATATCGTAGAGAAGCATCGTGCTAC |
| | | GCTTGACCCCAGCGCTCCAAGAGACTTTATCGATACGTACCTGCTTAGGAT |
| | | GGAAAAAGAGAAGTCAAATCATCACACAGAATTCCATCACGAAAACCTGAT |
| | | GATTTCCTTGCTATCTTTATTCTTCGCGGGGACCGAGACAAGTAGCACTAC |
| | | GCTAAGGTATGGGTTTTTACTAATGCTTAAGTACCCCCATGTAGCGGAGAA |
| | | AGTGCAAAAAGAAATAGATCAGGTCATAGGCTCACATAGACTTCCCACTTT |
| | | GGACGACCGTAGCAAGATGCCCTATACAGACGCTGTTATACATGAGATACA |
| | | ACGTTTCTCCGATTTGGTACCTATAGGGGTACCACATAGGGTGACGAAGGA |
| | | TACCATGTTTAGAGGGTACCTGTTACCGAAAAACACAGAGGTCTATCCTAT |
| | | TCTTTCTTCCGCCCTTCATGATCCACAATACTTTGACCACCCCGATTCCTTT |
| | | AACCCGGAACACTTCCTTGACGCTAATGGTGCATTAAAGAAAAGTGAGGCA |
| | | TTTATGCCATTCTCTACCGGCAAACGTATTTGCTTAGGGGAGGGCATCGCC |
| | | CGTAACGAGTTATTCTTATTCTTTACAACTATCCTGCAAAATTTCTCTGTA |
| | | TCCTCACATCTTGCCCCTAAGGACATTGACCTGACGCCTAAAGAGTCCGGT |
| | | ATCGGTAAGATTCCTCCTACTTATCAGATATGCTTTAGTGCGAGATGA |
| 166 | Rattus | MDLLSALTLETWVLLAVILVLLYRLGTHRHGIFKKQGIPGPKPLPFLGTVLNY |
| | norvegicus | YKGLGRFDMECYKKYGKIWGLFDGQTPVFAIMDTEMIKNVLVKECFSVFTN |
| | species of | RRDFGPVGIMGKAVSVAKDEEWKRYRALLSPTFTSGRLKEMFPIIEQYGDIL |
| | origin; amino | VKYLKQEAETGKPVTMKKVFGAYSMDVITSTSFGVNVDSLNNPKDPFVEKT |
| | acid sequence; | KKLLRFDFFDPLFLSVVLFPFLTPIYEMLNICMFPKDSIAFFQKFVHRIKETRL |
| | 14HC_P450_32 | DSKHKHRVDFLQLMLNAHNNSKDEVSHKALSDVEIIAQSVIFIFAGYETTSST |
| | | LSFVLYFLATHPDIQKKLQEEIDGALPSKAPPTYDIVMEMEYLDMVLNETLR |
| | | LYPIGNRLERVCKKDIELDGLFIPKGSVVTIPTYALHHDPQHWPKPEEFHPE |
| | | RFSKENKGSIDPYVYLPFGNGPRNCIGMRFALMNMKLALTKVLQNFSFQPC |
| | | KETQIPLKLSRQAILEPEKPIVLKVLPRDAVINGA* |
| 167 | Rattus | ATGGATCTTCTGTCCGCGCTAACTTTAGAAACGTGGGTACTACTGGCAGTA |
| | norvegicus | ATCCTAGTATTATTGTATAGGCTAGGGACTCACAGACACGGTATTTTCAAG |
| | species of | AAGCAAGGTATCCCAGGTCCGAAACCACTGCCCTTTCTGGGTACTGTCCTT |
| | origin; nucleic | AACTATTATAAAGGTCTGGGTAGGTTCGACATGGAATGCTATAAGAAGTAC |
| | acid sequence; | GGCAAAATTTGGGGCTTATTCGACGGGCAAACGCCCGTGTTTGCCATAATG |
| | 14HC_P450_32 | GATACCGAAATGATAAAGAATGTCCTAGTCAAAGAATGTTTCAGCGTTTTT |
| | | ACAAACAGGCGTGATTTTGGGCCTGTCGGAATTATGGGCAAGGCTGTAAGC |
| | | GTGGCTAAGGATGAAGAGTGGAAGAGATATAGAGCCTTACTGAGCCCTACC |
| | | TTCACTTCTGGTAGACTAAAAGAAATGTTTCCGATAATTGAGCAATACGGC |
| | | GACATCCTAGTTAAATACCTAAAACAAGAGGCCGAGACGGGCAAACCCGTG |
| | | ACTATGAAAAAGGTATTCGGAGCGTACAGTATGGATGTTATAACTTCTACC |
| | | TCTTTTGGGGTAAATGTCGACAGTTTAAACAACCCCAAAGACCCCTTCGTT |
| | | GAAAAAACTAAGAAATTATTAAGATTCGATTTTTTTGACCCTCTATTCTTA |
| | | TCCGTTGTATTATTCCCCTTCCTGACACCGATATATGAAATGTTAAATATC |
| | | TGTATGTTCCCGAAGGACAGTATAGCGTTTTTTCAAAAATTTGTCCATAGA |
| | | ATTAAGGAAACGCGTCTAGATAGCAAGCATAAGCACAGAGTTGACTTTTTG |
| | | CAACTAATGCTAAACGCTCATAACAACTCAAAAGACGAAGTGTCTCATAAA |
| | | GCGCTTAGTGACGTTGAAATAATCGCTCAGAGCGTCATATTTATATTTGCT |
| | | GGGTACGAAACCACCTCCTCAACTTTGTCTTTTGTCCTATATTTCCTTGCTA |
| | | CACATCCTGATATTCAAAAAAAGTTACAAGAAGAGATTGATGGGGCACTGC |
| | | CATCCAAGGCACCACCAACCTACGATATTGTAATGGAGATGGAATACCTAG |
| | | ACATGGTGTTGAACGAGACATTGAGATTGTATCCCATCGGTAATAGACTTG |
| | | AAAGAGTGTGCAAAAAAGACATTGAGCTTGATGGTCTTTTTATCCCGAAGG |
| | | GTTCCGTAGTGACAATCCCTACATACGCGCTACATCATGATCCTCAACATT |
| | | GGCCAAAGCCGGAGGAATTCCACCCCGAGCGTTTTTCCAAAGAAAACAAAG |
| | | GGAGTATAGACCCATATGTATATTTACCATTCGGGAACGGCCCACGTAACT |
| | | GTATCGGAATGAGGTTTGCCCTGATGAACATGAAATTAGCGTTGACCAAAG |
| | | TCCTTCAGAATTTCAGCTTCCAGCCTTGTAAAGAAACGCAAATCCCTCTGA |
| | | AGCTATCAAGACAAGCAATCCTGGAACCCGAAAAGCCAATTGTCTTAAAGG |
| | | TTCTACCGAGAGACGCGGTCATAAATGGTGCATGA |
| 178 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTLDALGKSVFGFEFGALDDPDS |
| | 14HC_P450_5 | VWVKTYRQVFDSFTDVFSLVFPRLDPIYRYFSAKHREQYNAVYKLIDLLDGM |
| | | ADKKRSMLQDASNSDIKDVPDHEKDLLQLMLEAELRGEGSWTKRELRHNM |
| | | AIFFVAGQDTTSHALTFCLYLLAKNQDIQKKAREEILNVFGDEPKDVFPTLE |
| | | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 179 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_5 | TTCCCTTTGCTATCTAAGTGTAATGGGGTATACCTTAATAGAATGCCATTT |
| | | AAGTGGACAATTTATGTCGCGGATCCAGAAATCGCGAGGGCGATTCTTTTT |
| | | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | TTAGACGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 180 | Lichtheimia | [symbol] [symbol] |
| | ramose species | [symbol] [symbol] |
| | of origin; amino | [symbol] [symbol] |
| | acid sequence; | [symbol] [symbol] |
| | 14HC_P450_33 | [symbol] [symbol] |
| | (14HC_P450_5: | [symbol] [symbol] |
| | A59D) | [symbol] [symbol] |
| | | [symbol] [symbol] |
| | | [symbol] [symbol] |
| | | [symbol] [symbol] |
| | | [symbol] [symbol] |
| | | [symbol] |
| 181 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_33 | TTCCCTTTGCTATCTAAGTGTAATGGGGTATACCTTAATAGAATGCCATTT |
| | (14HC_P450_5: | AAGTGGACAATTTATGTCGCGGATCCAGAAATCGCGAGGGCGATTCTTTTT |
| | A59D) | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | TTAGACGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 182 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTIDALGKSVFGFEFGALDDPDS |
| | 14HC_P450_34 | VWVKTYRQVFDSFTDVFSLVFPRLDPIYRYFSAKHREQYNAVYKLIDLLDGM |
| | (14HC_P450_5: | ADKKRSMLQDASNSDIKDVPDHEKDLLQLMLEAELRGEGSWTKRELRHNM |
| | L188I) | AIFFVAGQDTTSHALTFCLYLLAKNQDIQKKAREEILNVFGDEPKDVFPTLE |
| | | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 183 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_34 | TTCCCTTTGCTATCTAAGTGTAATGGGGTATACCTTAATAGAATGCCATTT |
| | (14HC_P450_5: | AAGTGGACAATTTATGTCGCGGATCCAGAAATCGCGAGGGCGATTCTTTTT |
| | L188I) | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | aTAGACGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 184 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTIEALGKSVFGFEFGALDDPDSV |
| | 14HC_P450_35 | WVKTYRQVFDSFTDVFSLVFPRLDPIYRYFSAKHREQYNAVYKLIDLLDGMA |
| | (14HC_P450_5: | DKKRSMLQDASNSDIKDVPDHEKDLLQLMLEAELRGEGSWTKRELRHNMA |
| | L188I, D189E) | IFFVAGQDTTSHALTFCLYLLAKNQDIQKKAREEILNVFGDEPKDVFPTLEDC |
| | | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 185 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_35 | TTCCCTTTGCTATCTAAGTGTAATGGGGTATACCTTAATAGAATGCCATTT |
| | (14HC_P450_5: | AAGTGGACAATTTATGTCGCGGATCCAGAAATCGCGAGGGCGATTCTTTTT |
| | L188I, D189E) | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | aTAGAgGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 186 | Lichtheimia | MTEIKEHIYRYRHYIGVAAAVALVCQQVYYRIFRIPKNLRHIPAIPYGQQLKA |
| | ramose species | LRSDEDLTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTIEALGKSVFGFEFGALDDPDSV |
| | 14HC_P450_36 | WVKTYRQVFDSFTDVFSLVFPRLDPIYRYFSAKHREQYNAVYKLIDLLDGMA |
| | (14HC_P450_5: | DKKRSMLQDASNSDIKDVPDHEKDLLQLMLEAELRGEGSWTKRELRHNMA |
| | L188I, D189E, | IFFVAGQDTTSHALTFCLYLLAKNQDIQKKAREEILNVFGDEPKDVFPTLEDC |
| | A59D) | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 187 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_36 | TTCCCTTTGCTATCTAAGTGTAATGGGGTATACCTTAATAGAATGCCATTT |
| | (14HC_P450_5: | AAGTGGACAATTTATGTCGCGGATCCAGAAATCGCGAGGGCGATTCTTTTT |
| | L188I, D189E, | AAACCCGAATTCGGCCACAAGACCAGGAGTGTGACAGATTCCTTGGATAAG |
| | A59D) | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | aTAGAgGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 188 | Lichtheimia | MTEIKEHIYRYRHYIGVAAAVALVCQQVYYRIFRIPKNLRHIPAIPYGQQLKA |
| | ramose species | LRSDEDLTSRTKRLVFPLLSKCNGVYLNRMPFKWTIYVADPEIARAILLKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_37 | WVKTYRQVFDSFTDVFSLVFPRLDPIYRYFSAKHREQYNAVYKLIDLLDGMA |
| | (A59D, F102L, | DKKRSMLQDASNSDIKDVPDHEKDLLQLMLEAELRGEGSWTKRELRHNMA |
| | D181E, L188I, | IFFVAGQDTTSHAMTFCLYLLAKNQDIQKKAREEILNVFGDEPKDVFPTLED |
| | D189E, S208C, | CKKLNYLDMVIKESMRIYPPANDVLARDVNEDLNVKGVFIPKGSMVSVDIH |
| | L325M) | ALHHRPDLWHEPDKFNPDRFLPGGEHDSHVGVTYAPFSSGSRQCIALKFAT |
| | | MQQRVVLSMLLRKYEWELPKDSKHKDSIQFQIPFNIAPKDLELTFHKRY* |
| 189 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_37 | TTCCCTTTGCTATCTAAGTGTAATGGGGTATACCTTAATAGAATGCCATTT |
| | (A59D, F102L, | AAGTGGACAATTTATGTCGCGGATCCAGAAATCGCGAGGGCGATTCTTTTa |
| | D181E, L188I, | AAACCCGAATTCGGCCACAAGACCAGGAGTGTGACAGATTCCTTGGATAAG |
| | D189E, S208C, | AACTCAAGCCTATTTGAATTTGTTGGGGACGATAATATAGCCATAGTTAAT |
| | L325M) | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTaTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 190 | Lichtheimia | MTEIKEHIYRYRHYIGVAAAVALVCQQVYYRIFRIPKNLRHIPAIPYGQQLKA |
| | ramose species | LRSDKDLTSRTKRLVFPLLSKCNGVYLNRMPFKWTIYVADPEIARAILLKPE |
| | of origin; amino | FGHKTRSVTDSLDKNSSLFEFVGDDNIAIVNGHEWKEQRKIMNPAFHRATP |
| | acid sequence; | VGMFGSLMPKVFRLVEEQPTLPALELMQKLTIEALGKSVFGFEFGALDDPDS |
| | 14HC_P450_39 | VWVKTYRQVFDSFTDVFSLVFPRLDPIYRYFSAKHREQYNAVYKLIDLLDGM |
| | (E58K, A59D, | ADKKRSMLQDASNSDIKDVPDHEKDLLQLMLEAELRGEGSWTKRELRHNM |
| | F102L, D181E, | AIFFVAGQDTTSHALTFCLYLLAKNQDIQKKAREEILNVFGDEPKDVFPTLE |
| | L188I, D189E) | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 191 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATaagGaTCTTACCAGCAGGACGAAGCGTTTAGTAT |
| | 14HC_P450_39 | TCCCTTTGCTATCTAAGTGTAATGGGGTATACCTTAATAGAATGCCATTTA |
| | (E58K, A59D, | AGTGGACAATTTATGTCGCGGATCCAGAAATCGCGAGGGCGATTCTTTTaA |
| | F102L, D181E, | AACCCGAATTCGGCCACAAGACCAGGAGTGTGACAGATTCCTTGGATAAGA |
| | L188I, D189E) | ACTCAAGCCTATTTGAATTTGTTGGGGACGATAATATAGCCATAGTTAATG |
| | | GCCATGAATGGAAAGAGCAACGTAAGATAATGAACCCAGCATTCCACAGGG |
| | | CTACGCCCGTAGGGATGTTCGGCAGTTTAATGCCTAAAGTTTTTAGGCTTG |
| | | TGGAAGAACAGCCTACACTACCCGCCCTAGAaTTAATGCAAAAGTTAACGa |
| | | TAGAgGCACTTGGGAAGTCAGTATTCGGCTTTGAATTCGGCGCACTAGATG |
| | | ACCCCGACAGTGTGTGGGTTAAGACTTATAGACAAGTGTTTGACTCTTTTA |
| | | CCGACGTTTTCTCTCTAGTGTTTCCCCGTTTAGATCCAATCTACCGTTATTT |
| | | TTCTGCGAAACACAGAGAACAGTACAACGCTGTTTACAAACTAATTGATCT |
| | | GCTGGATGGAATGGCAGACAAGAAAAGGAGCATGTTGCAAGATGCCTCCAA |
| | | CTCCGATATCAAAGATGTTCCAGACCACGAGAAAGATTTACTGCAACTTAT |
| | | GTTGGAGGCTGAACTGCGTGGCGAAGGCTCATGGACAAAACGTGAATTGCG |
| | | TCATAACATGGCCATATTTTTTGTGGCAGGTCAGGACACAACGAGTCATGC |
| | | TTTGACTTTTTGCCTTTATTTACTTGCGAAGAATCAAGATATCCAGAAGAA |
| | | GGCGAGAGAAGAAATCTTAAATGTTTTTGGGGATGAACCTAAAGACGTGTT |
| | | TCCCACACTTGAAGATTGCAAAAAACTAAATTATCTTGACATGGTGATCAA |
| | | GGAATCCATGAGAATTTACCCTCCTGCTAACGATGTCCTAGCCAGGGATGT |
| | | CAATGAAGATTTAAACGTCAAGGGTGTCTTCATTCCCAAGGGATCTATGGT |
| | | ATCAGTGGACATTCATGCCCTACACCATAGACCAGACCTGTGGCATGAGCC |
| | | CGATAAGTTCAATCCCGATCGTTTCCTGCCGGGTGGGGAGCATGACAGTCA |
| | | CGTCGGCGTAACATACGCCCCCTTCTCATCCGGCTCCAGACAGTGCATCGCG |
| | | TTAAAATTCGCTACCATGCAGCAAAGGGTAGTCCTGTCCATGTTGCTGAGA |
| | | AAGTACGAATGGGAATTGCCAAAGGATTCCAAACATAAGGACTCCATCCAG |
| | | TTCCAGATTCCCTTCAACATCGCTCCTAAGGATCTAGAGCTTACCTTTCATA |
| | | AACGTTACTAG |
| 194 | Lichtheimia | MTEIKEHIYRYRHYIGVAAAVALVCQQVYYRIFRIPKNLRHIPAIPYGQQLKA |
| | ramose species | LRSDKDLTSRTKRLVFPLLSKCNGVYLNRMPFKWTIYVADPEIARAILFKPE |
| | of origin; amino | FGHKTRSVTDSLDKNSSLFEFVGDDNIAIVNGHEWKEQRKIMNPAFHRATP |
| | acid sequence; | VGMFGSLMPKVFRLVEEQPTLPALELMQKLTIEALGKSVFGFEFGALDDPDC |
| | 14HC_P450_41 | VWVKTYRQVFDSFTDVFSLVFPRLDPIYRYFSAKHREQYNAVYKLIDLLDGM |
| | (E58K, A59D, | ADKKRSMLQDASNSDIKDVPDHEKDLLQLMLEAELRGEGSWTKRELRHNM |
| | D181E, L188I, | AIFFVAGQDTTSHALTFCLYLLAKNQDIQKKAREEILNVFGDEPKDVFPTLE |
| | D189E, S208C) | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 195 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATaagGaTCTTACCAGCAGGACGAAGCGTTTAGTAT |
| | 14HC_P450_41 | TCCCTTTGCTATCTAAGTGTAATGGGGTATACCTTAATAGAATGCCATTTA |
| | (E58K, A59D, | AGTGGACAATTTATGTCGCGGATCCAGAAATCGCGAGGGCGATTCTTTTTA |
| | D181E, L188I, | AACCCGAATTCGGCCACAAGACCAGGAGTGTGACAGATTCCTTGGATAAGA |
| | D189E, S208C) | ACTCAAGCCTATTTGAATTTGTTGGGGACGATAATATAGCCATAGTTAATG |
| | | GCCATGAATGGAAAGAGCAACGTAAGATAATGAACCCAGCATTCCACAGGG |
| | | CTACGCCCGTAGGGATGTTCGGCAGTTTAATGCCTAAAGTTTTTAGGCTTG |
| | | TGGAAGAACAGCCTACACTACCCGCCCTAGAaTTAATGCAAAAGTTAACGa |
| | | TAGAgGCACTTGGGAAGTCAGTATTCGGCTTTGAATTCGGCGCACTAGATG |
| | | ACCCCGACtGTGTGTGGGTTAAGACTTATAGACAAGTGTTTGACTCTTTTAC |
| | | CGACGTTTTCTCTCTAGTGTTTCCCCGTTTAGATCCAATCTACCGTTATTTT |
| | | TCTGCGAAACACAGAGAACAGTACAACGCTGTTTACAAACTAATTGATCTG |
| | | CTGGATGGAATGGCAGACAAGAAAAGGAGCATGTTGCAAGATGCCTCCAAC |
| | | TCCGATATCAAAGATGTTCCAGACCACGAGAAAGATTTACTGCAACTTATG |
| | | TTGGAGGCTGAACTGCGTGGCGAAGGCTCATGGACAAAACGTGAATTGCGT |
| | | CATAACATGGCCATATTTTTTGTGGCAGGTCAGGACACAACGAGTCATGCT |
| | | TTGACTTTTTGCCTTTATTTACTTGCGAAGAATCAAGATATCCAGAAGAAG |
| | | GCGAGAGAAGAAATCTTAAATGTTTTTGGGGATGAACCTAAAGACGTGTTT |
| | | CCCACACTTGAAGATTGCAAAAAACTAAATTATCTTGACATGGTGATCAAG |
| | | GAATCCATGAGAATTTACCCTCCTGCTAACGATGTCCTAGCCAGGGATGTC |
| | | AATGAAGATTTAAACGTCAAGGGTGTCTTCATTCCCAAGGGATCTATGGTA |
| | | TCAGTGGACATTCATGCCCTACACCATAGACCAGACCTGTGGCATGAGCCC |
| | | GATAAGTTCAATCCCGATCGTTTCCTGCCGGGTGGGGAGCATGACAGTCAC |
| | | GTCGGCGTAACATACGCCCCCTTCTCATCCGGCTCCAGACAGTGCATCGCGT |
| | | TAAAATTCGCTACCATGCAGCAAAGGGTAGTCCTGTCCATGTTGCTGAGAA |
| | | AGTACGAATGGGAATTGCCAAAGGATTCCAAACATAAGGACTCCATCCAGT |
| | | TCCAGATTCCCTTCAACATCGCTCCTAAGGATCTAGAGCTTACCTTTCATA |
| | | AACGTTACTAG |
| 196 | Lichtheimia | MTEIKEHIYRYRHYIGIAAAVALVCQQVYYRIFRIPKNLRHIPAIPYGQQLKAL |
| | ramose species | RSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTLDALGKSVFGFEFGALDDPDS |
| | 14HC_P450_43 | VWVKTYRQVFDSFTDVFSLVFPRLDPIYRYFSAKHREQYNAVYKLIDLLDGM |
| | (V17I) | ADKKRSMLQDASNSDIKDVPDHEKDLLQLMLEAELRGEGSWTKRELRHNM |
| | | AIFFVAGQDTTSHALTFCLYLLAKNQDIQKKAREEILNVFGDEPKDVFPTLE |
| | | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 197 | Lichtheimia | ATGACAGAGATCAAGGAGCACATCTACAGATACCGTCACTATATCGGGaTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_43 | TTCCCTTTGCTATCTAAGTGTAATGGGGTATACCTTAATAGAATGCCATTT |
| | (V17I) | AAGTGGACAATTTATGTCGCGGATCCAGAAATCGCGAGGGCGATTCTTTTT |
| | | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | TTAGACGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 198 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILLKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTLDALGKSVFGFEFGALDDPDS |
| | 14HC_P450_44 | VWVKTYRQVFDSFTDVFSLVFPRLDPIYRYFSAKHREQYNAVYKLIDLLDGM |
| | (F102L) | ADKKRSMLQDASNSDIKDVPDHEKDLLQLMLEAELRGEGSWTKRELRHNM |
| | | AIFFVAGQDTTSHALTFCLYLLAKNQDIQKKAREEILNVFGDEPKDVFPTLE |
| | | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 199 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_44 | TTCCCTTTGCTATCTAAGTGTAATGGGGTATACCTTAATAGAATGCCATTT |
| | (F102L) | AAGTGGACAATTTATGTCGCGGATCCAGAAATCGCGAGGGCGATTCTTTTa |
| | | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | TTAGACGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 200 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTLDALGKSVFGFEFGALDDPDS |
| | 14HC_P450_45 | VWVKTYRQVFDSFTDVFSLVFPRLDPIYRYFSAKHREQYNAVYKLIDLLDGM |
| | (D181E) | ADKKRSMLQDASNSDIKDVPDHEKDLLQLMLEAELRGEGSWTKRELRHNM |
| | | AIFFVAGQDTTSHALTFCLYLLAKNQDIQKKAREEILNVFGDEPKDVFPTLE |
| | | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 201 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_45 | TTCCCTTTGCTATCTAAGTGTAATGGGGTATACCTTAATAGAATGCCATTT |
| | (D181E) | AAGTGGACAATTTATGTCGCGGATCCAGAAATCGCGAGGGCGATTCTTTTT |
| | | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | TTAGACGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 202 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTLEALGKSVFGFEFGALDDPDS |
| | 14HC_P450_46 | VWVKTYRQVFDSFTDVFSLVFPRLDPIYRYFSAKHREQYNAVYKLIDLLDGM |
| | (D189E) | ADKKRSMLQDASNSDIKDVPDHEKDLLQLMLEAELRGEGSWTKRELRHNM |
| | | AIFFVAGQDTTSHALTFCLYLLAKNQDIQKKAREEILNVFGDEPKDVFPTLE |
| | | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 203 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_46 | TTCCCTTTGCTATCTAAGTGTAATGGGGTATACCTTAATAGAATGCCATTT |
| | (D189E) | AAGTGGACAATTTATGTCGCGGATCCAGAAATCGCGAGGGCGATTCTTTTT |
| | | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | TTAGAgGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTA |
| 204 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTLDALGKSVFGFEFGALDDPDC |
| | 14HC_P450_47 | VWVKTYRQVFDSFTDVFSLVFPRLDPIYRYFSAKHREQYNAVYKLIDLLDGM |
| | (S280C) | ADKKRSMLQDASNSDIKDVPDHEKDLLQLMLEAELRGEGSWTKRELRHNM |
| | | AIFFVAGQDTTSHALTFCLYLLAKNQDIQKKAREEILNVFGDEPKDVFPTLE |
| | | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 205 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_47 | TTCCCTTTGCTATCTAAGTGTAATGGGGTATACCTTAATAGAATGCCATTT |
| | (S280C) | AAGTGGACAATTTATGTCGCGGATCCAGAAATCGCGAGGGCGATTCTTTTT |
| | | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | TTAGACGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 206 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTLDALGKSVFGFEFGALDDPDS |
| | 14HC_P450_48 | VWVKTYRQVFDSFTDVFSLVFPRLDPIYRYFSAKHREQYNAVYKLIDLLDGM |
| | (L325M) | ADKKRSMLQDASNSDIKDVPDHEKDLLQLMLEAELRGEGSWTKRELRHNM |
| | | AIFFVAGQDTTSHAMTFCLYLLAKNQDIQKKAREEILNVFGDEPKDVFPTLE |
| | | DCKKLNYLDMVIKESMRIYPPANDVLARDVNEDLNVKGVFIPKGSMVSVDI |
| | | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 207 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_48 | TTCCCTTTGCTATCTAAGTGTAATGGGGTATACCTTAATAGAATGCCATTT |
| | (L325M) | AAGTGGACAATTTATGTCGCGGATCCAGAAATCGCGAGGGCGATTCTTTTT |
| | | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | TTAGACGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTaTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 208 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTLDALGKSVFGFEFGALDDPDS |
| | 14HC_P450_49 | VWVKTYRQVFDSFTDVFSLVFPRLDPIYRYFSAKHREQYNAVYKLIDLLDGM |
| | (L396I) | ADKKRSMLQDASNSDIKDVPDHEKDLLQLMLEAELRGEGSWTKRELRHNM |
| | | AIFFVAGQDTTSHALTFCLYLLAKNQDIQKKAREEILNVFGDEPKDVFPTLE |
| | | DCKKLNYLDMVIKESMRIYPPANDVLARDVNEDINVKGVFIPKGSMVSVDIH |
| | | ALHHRPDLWHEPDKFNPDRFLPGGEHDSHVGVTYAPFSSGSRQCIALKFAT |
| | | MQQRVVLSMLLRKYEWELPKDSKHKDSIQFQIPFNIAPKDLELTFHKRY* |
| 209 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_49 | TTCCCTTTGCTATCTAAGTGTAATGGGGTATACCTTAATAGAATGCCATTT |
| | (L396I) | AAGTGGACAATTTATGTCGCGGATCCAGAAATCGCGAGGGCGATTCTTTTT |
| | | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | TTAGACGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATATAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 210 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTIEALGKSVFGFEFGALDDPDCV |
| | 14HC_P450_50 | WVKTYRQVFDSFTDVFSLVFPRLDPIYRYFSAKHREQYNAVYKLIDLLDGMA |
| | (L188I, D189E, | DKKRSMLQDASNSDIKDVPDHEKDLLQLMLEAELRGEGSWTKRELRHNMA |
| | S208C) | IFFVAGQDTTSHALTFCLYLLAKNQDIQKKAREEILNVFGDEPKDVFPTLEDC |
| | | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 211 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_50 | TTCCCTTTGCTATCTAAGTGTAATGGGGTATACCTTAATAGAATGCCATTT |
| | (L188I, D189E, | AAGTGGACAATTTATGTCGCGGATCCAGAAATCGCGAGGGCGATTCTTTTT |
| | S208C) | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 212 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILLKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTIEALGKSVFGFEFGALDDPDSV |
| | 14HC_P450_51 | WVKTYRQVFDSFTDVFSLVFPRLDPIYRYFSAKHREQYNAVYKLIDLLDGMA |
| | (F102L, L188I, | DKKRSMLQDASNSDIKDVPDHEKDLLQLMLEAELRGEGSWTKRELRHNMA |
| | D189E) | IFFVAGQDTTSHALTFCLYLLAKNQDIQKKAREEILNVFGDEPKDVFPTLEDC |
| | | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 213 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_51 | TTCCCTTTGCTATCTAAGTGTAATGGGGTATACCTTAATAGAATGCCATTT |
| | (F102L, L188I, | AAGTGGACAATTTATGTCGCGGATCCAGAAATCGCGAGGGCGATTCTTTTa |
| | D189E) | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | aTAGAgGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 214 | Lichtheimia | MTEIKEHIYRYRHYIGIAAAVALVCQQVYYRIFRIPKNLRHIPAIPYGQQLKAL |
| | ramose species | RSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTIEALGKSVFGFEFGALDDPDSV |
| | 14HC_P450_52 | WVKTYRQVFDSFTDVFSLVFPRLDPIYRYFSAKHREQYNAVYKLIDLLDGMA |
| | (V17I, L1881, | DKKRSMLQDASNSDIKDVPDHEKDLLQLMLEAELRGEGSWTKRELRHNMA |
| | D189E) | IFFVAGQDTTSHALTFCLYLLAKNQDIQKKAREEILNVFGDEPKDVFPTLEDC |
| | | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 215 | Lichtheimia | ATGACAGAGATCAAGGAGCACATCTACAGATACCGTCACTATATCGGGaTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_52 | TTCCCTTTGCTATCTAAGTGTAATGGGGTATACCTTAATAGAATGCCATTT |
| | (V17I, L1881, | AAGTGGACAATTTATGTCGCGGATCCAGAAATCGCGAGGGCGATTCTTTTT |
| | D189E) | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | aTAGAgGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 216 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALDLMQKLTIEALGKSVFGFEFGALDDPDSV |
| | 14HC_P450_53 | WVKTYRQVFDSFTDVFSLVFPRLDPIYRYFSAKHREQYNAVYKLIDLLDGMA |
| | (L188I, D189E, | DKKRSMLQDASNSDIKDVPDHEKDLLQLMLEAELRGEGSWTKRELRHNMA |
| | L325M) | IFFVAGQDTTSHAMTFCLYLLAKNQDIQKKAREEILNVFGDEPKDVFPTLED |
| | | CKKLNYLDMVIKESMRIYPPANDVLARDVNEDLNVKGVFIPKGSMVSVDIH |
| | | ALHHRPDLWHEPDKFNPDRFLPGGEHDSHVGVTYAPFSSGSRQCIALKFAT |
| | | MQQRVVLSMLLRKYEWELPKDSKHKDSIQFQIPFNIAPKDLELTFHKRY* |
| 217 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_53 | TTCCCTTTGCTATCTAAGTGTAATGGGGTATACCTTAATAGAATGCCATTT |
| | (L188I, D189E, | AAGTGGACAATTTATGTCGCGGATCCAGAAATCGCGAGGGCGATTCTTTTT |
| | L325M) | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGATTTAATGCAAAAGTTAACG |
| | | aTAGAgGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTaTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 218 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDSV |
| | 14HC_P450_54 | WVKTYRQVFDSFTDVFSLVFPRLDPIYRYFSAKHREQYNAVYKLIDLLDGMA |
| | (D181E, L188I, | DKKRSMLQDASNSDIKDVPDHEKDLLQLMLEAELRGEGSWTKRELRHNMA |
| | D189E) | IFFVAGQDTTSHALTFCLYLLAKNQDIQKKAREEILNVFGDEPKDVFPTLEDC |
| | | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 219 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_54 | TTCCCTTTGCTATCTAAGTGTAATGGGGTATACCTTAATAGAATGCCATTT |
| | (D181E, L188I, | AAGTGGACAATTTATGTCGCGGATCCAGAAATCGCGAGGGCGATTCTTTTT |
| | D189E) | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 220 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_55 | WVKTYRQVFDSFTDVFSLVFPRLDPIYRYFSAKHREQYNAVYKLIDLLDGMA |
| | (D181E, L188I, | DKKRSMLQDASNSDIKDVPDHEKDLLQLMLEAELRGEGSWTKRELRHNMA |
| | D189E, S208C) | IFFVAGQDTTSHALTFCLYLLAKNQDIQKKAREEILNVFGDEPKDVFPTLEDC |
| | | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 221 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_55 | TTCCCTTTGCTATCTAAGTGTAATGGGGTATACCTTAATAGAATGCCATTT |
| | (D181E, L188I, | AAGTGGACAATTTATGTCGCGGATCCAGAAATCGCGAGGGCGATTCTTTTT |
| | D189E, S208C) | AAACCCGAATTCGGCCACAAGACCAGGAGTGTGACAGATTCCTTGGATAAG |
| | | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 222 | Lichtheimia | MTEIKEHIYRYRHYIGVAAAVALVCQQVYYRIFRIPKNLRHIPAIPYGQQLKA |
| | ramose species | LRSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_56 | WVKTYRQVFDSFTDVFSLVFPRLDPIYRYFSAKHREQYNAVYKLIDLLDGMA |
| | (D181E, L188I, | DKKRSMLQDASNSDIKDVPDHEKDLLQLMLEAELRGEGSWTKRELRHNMA |
| | D189E, S208C, | IFFVAGQDTTSHAMTFCLYLLAKNQDIQKKAREEILNVFGDEPKDVFPTLED |
| | L325M) | CKKLNYLDMVIKESMRIYPPANDVLARDVNEDLNVKGVFIPKGSMVSVDIH |
| | | ALHHRPDLWHEPDKFNPDRFLPGGEHDSHVGVTYAPFSSGSRQCIALKFAT |
| | | MQQRVVLSMLLRKYEWELPKDSKHKDSIQFQIPFNIAPKDLELTFHKRY* |
| 223 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_56 | TTCCCTTTGCTATCTAAGTGTAATGGGGTATACCTTAATAGAATGCCATTT |
| | (D181E, L188I, | AAGTGGACAATTTATGTCGCGGATCCAGAAATCGCGAGGGCGATTCTTTTT |
| | D189E, S208C, | AAACCCGAATTCGGCCACAAGACCAGGAGTGTGACAGATTCCTTGGATAAG |
| | L325M) | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTaTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 224 | Lichtheimia | MTEIKEHIYRYRHYIGIAAAVALVCQQVYYRIFRIPKNLRHIPAIPYGQQLKAL |
| | ramose species | RSDEALTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_57 | WVKTYRQVFDSFTDVFSLVFPRLDPIYRYFSAKHREQYNAVYKLIDLLDGMA |
| | (V17I, D181E, | DKKRSMLQDASNSDIKDVPDHEKDLLQLMLEAELRGEGSWTKRELRHNMA |
| | L188I, D189E, | IFFVAGQDTTSHALTFCLYLLAKNQDIQKKAREEILNVFGDEPKDVFPTLEDC |
| | S208C) | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 225 | Lichtheimia | ATGACAGAGATCAAGGAGCACATCTACAGATACCGTCACTATATCGGGaTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGCTCTTACCAGCAGGACGAAGCGTTTAGTA |
| | 14HC_P450_57 | TTCCCTTTGCTATCTAAGTGTAATGGGGTATACCTTAATAGAATGCCATTT |
| | (V17I, D181E, | AAGTGGACAATTTATGTCGCGGATCCAGAAATCGCGAGGGCGATTCTTTTT |
| | L188I, D189E, | AAACCCGAATTCGGCCACAAGACCAGGAGTGTGACAGATTCCTTGGATAAG |
| | S208C) | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 226 | Lichtheimia | MTEIKEHIYRYRHYIGVAAAVALVCQQVYYRIFRIPKNLRHIPAIPYGQQLKA |
| | ramose species | LRSDEDLTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_58 | WVKTYRQVFDSFTDVFSLVFPRLDPIYRYFSAKHREQYNAVYKLIDLLDGMA |
| | (A59D, D181E, | DKKRSMLQDASNSDIKDVPDHEKDLLQLMLEAELRGEGSWTKRELRHNMA |
| | L188I, D189E, | IFFVAGQDTTSHALTFCLYLLAKNQDIQKKAREEILNVFGDEPKDVFPTLEDC |
| | S208C) | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 227 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_58 | TTCCCTTTGCTATCTAAGTGTAATGGGGTATACCTTAATAGAATGCCATTT |
| | (A59D, D181E, | AAGTGGACAATTTATGTCGCGGATCCAGAAATCGCGAGGGCGATTCTTTTT |
| | L188I, D189E, | AAACCCGAATTCGGCCACAAGACCAGGAGTGTGACAGATTCCTTGGATAAG |
| | S208C) | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 228 | Lichtheimia | MTEIKEHIYRYRHYIGVAAAVALVCQQVYYRIFRIPKNLRHIPAIPYGQQLKA |
| | ramose species | LRSDEDLTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_59 | WVKTYRQVFDSFTDVFSLVFPRLDPIYRYFSAKHREQYNAVYKLIDLLDGMA |
| | (A59D, D181E, | DKKRSMLQDASNSDIKDVPDHEKDLLQLMLEAELRGEGSWTKRELRHNMA |
| | L188I, D189E, | IFFVAGQDTTSHAMTFCLYLLAKNQDIQKKAREEILNVFGDEPKDVFPTLED |
| | S208C, L325M) | CKKLNYLDMVIKESMRIYPPANDVLARDVNEDLNVKGVFIPKGSMVSVDIH |
| | | ALHHRPDLWHEPDKFNPDRFLPGGEHDSHVGVTYAPFSSGSRQCIALKFAT |
| | | MQQRVVLSMLLRKYEWELPKDSKHKDSIQFQIPFNIAPKDLELTFHKRY* |
| 229 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_59 | TTCCCTTTGCTATCTAAGTGTAATGGGGTATACCTTAATAGAATGCCATTT |
| | (A59D, D181E, | AAGTGGACAATTTATGTCGCGGATCCAGAAATCGCGAGGGCGATTCTTTTT |
| | L188I, D189E, | AAACCCGAATTCGGCCACAAGACCAGGAGTGTGACAGATTCCTTGGATAAG |
| | S208C, L325M) | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTaTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 230 | Lichtheimia | MTEIKEHIYRYRHYIGVAAAVALVCQQVYYRIFRIPKNLRHIPAIPYGQQLKA |
| | ramose species | LRSDEDLTSRTKRLVFPLLSKCNGVYLNRMPFKWTIYVADPEIARAILLKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_60 | WVKTYRQVFDSFTDVFSLVFPRLDPIYRYFSAKHREQYNAVYKLIDLLDGMA |
| | (A59D, F102L, | DKKRSMLQDASNSDIKDVPDHEKDLLQLMLEAELRGEGSWTKRELRHNMA |
| | D181E, L188I, | IFFVAGQDTTSHALTFCLYLLAKNQDIQKKAREEILNVFGDEPKDVFPTLEDC |
| | D189E, S208C) | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 231 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_60 | TTCCCTTTGCTATCTAAGTGTAATGGGGTATACCTTAATAGAATGCCATTT |
| | (A59D, F102L, | AAGTGGACAATTTATGTCGCGGATCCAGAAATCGCGAGGGCGATTCTTTTa |
| | D181E, L188I, | AAACCCGAATTCGGCCACAAGACCAGGAGTGTGACAGATTCCTTGGATAAG |
| | D189E, S208C) | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 232 | Lichtheimia | MTEIKEHIYRYRHYIGIAAAVALVCQQVYYRIFRIPKNLRHIPAIPYGQQLKAL |
| | ramose species | RSDEDLTSRTKRLVFPLLSKCNGVYLNRMPFKWTIYVADPEIARAILFKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_61 | WVKTYRQVFDSFTDVFSLVFPRLDPIYRYFSAKHREQYNAVYKLIDLLDGMA |
| | (V17I, A59D, | DKKRSMLQDASNSDIKDVPDHEKDLLQLMLEAELRGEGSWTKRELRHNMA |
| | L188I, D189E, | IFFVAGQDTTSHAMTFCLYLLAKNQDIQKKAREEILNVFGDEPKDVFPTLED |
| | S208C, D181E, | CKKLNYLDMVIKESMRIYPPANDVLARDVNEDLNVKGVFIPKGSMVSVDIH |
| | L325M) | ALHHRPDLWHEPDKFNPDRFLPGGEHDSHVGVTYAPFSSGSRQCIALKFAT |
| | | MQQRVVLSMLLRKYEWELPKDSKHKDSIQFQIPFNIAPKDLELTFHKRY* |
| 233 | Lichtheimia | ATGACAGAGATCAAGGAGCACATCTACAGATACCGTCACTATATCGGGaTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_61 | TTCCCTTTGCTATCTAAGTGTAATGGGGTATACCTTAATAGAATGCCATTT |
| | (V17I, A59D, | AAGTGGACAATTTATGTCGCGGATCCAGAAATCGCGAGGGCGATTCTTTTT |
| | L188I, D189E, | AAACCCGAATTCGGCCACAAGACCAGGAGTGTGACAGATTCCTTGGATAAG |
| | S208C, D181E, | AACTCAAGCCTATTTGAATTTGTTGGGGACGATAATATAGCCATAGTTAAT |
| | L325M) | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTaTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 234 | Lichtheimia | MTEIKEHIYRYRHYIGIAAAVALVCQQVYYRIFRIPKNLRHIPAIPYGQQLKAL |
| | ramose species | RSDEDLTSRTKRLVFPLLSKCNGVYLNRMPFKWTIYVADPEIARAILLKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_62 | WVKTYRQVFDSFTDVFSLVFPRLDPIYRYFSAKHREQYNAVYKLIDLLDGMA |
| | (V17I, A59D, | DKKRSMLQDASNSDIKDVPDHEKDLLQLMLEAELRGEGSWTKRELRHNMA |
| | F102L, D181E, | IFFVAGQDTTSHALTFCLYLLAKNQDIQKKAREEILNVFGDEPKDVFPTLEDC |
| | L188I, D189E, | KKLNYLDMVIKESMRIYPPANDVLARDVNEDLNVKGVFIPKGSMVSVDIHAL |
| | S208C) | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 235 | Lichtheimia | ATGACAGAGATCAAGGAGCACATCTACAGATACCGTCACTATATCGGGaTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_62 | TTCCCTTTGCTATCTAAGTGTAATGGGGTATACCTTAATAGAATGCCATTT |
| | (V17I, A59D, | AAGTGGACAATTTATGTCGCGGATCCAGAAATCGCGAGGGCGATTCTTTTa |
| | F102L, D181E, | AAACCCGAATTCGGCCACAAGACCAGGAGTGTGACAGATTCCTTGGATAAG |
| | L188I, D189E, | AACTCAAGCCTATTTGAATTTGTTGGGGACGATAATATAGCCATAGTTAAT |
| | S208C) | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 236 | Lichtheimia | MTEIKEHIYRYRHYIGVAAAVALVCQQVYYRIFRIPKNLRHIPAIPYGQQLKA |
| | ramose species | LRSDEDLTSRTKRLVFPLLSKCNGVYLNRMPFKWTIYVADPEIARAILLKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDSV |
| | 14HC_P450_63 | WVKTYRQVFDSFTDVFSLVFPRLDPIYRYFSAKHREQYNAVYKLIDLLDGMA |
| | (A59D, F102L, | DKKRSMLQDASNSDIKDVPDHEKDLLQLMLEAELRGEGSWTKRELRHNMA |
| | D181E, L188I, | IFFVAGQDTTSHALTFCLYLLAKNQDIQKKAREEILNVFGDEPKDVFPTLEDC |
| | D189E) | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 237 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_63 | TTCCCTTTGCTATCTAAGTGTAATGGGGTATACCTTAATAGAATGCCATTT |
| | (A59D, F102L, | AAGTGGACAATTTATGTCGCGGATCCAGAAATCGCGAGGGCGATTCTTTTa |
| | D181E, L188I, | AAACCCGAATTCGGCCACAAGACCAGGAGTGTGACAGATTCCTTGGATAAG |
| | D189E) | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 238 | Lichtheimia | MTEIKEHIYRYRHYIGIAAAVALVCQQVYYRIFRIPKNLRHIPAIPYGQQLKAL |
| | ramose species | RSDEDLTSRTKRLVFPLLSKCNGVYLNRMPFKWTIYVADPEIARAILLKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDSV |
| | 14HC_P450_64 | WVKTYRQVFDSFTDVFSLVFPRLDPIYRYFSAKHREQYNAVYKLIDLLDGMA |
| | (V17I, A59D, | DKKRSMLQDASNSDIKDVPDHEKDLLQLMLEAELRGEGSWTKRELRHNMA |
| | F102L, D181E, | IFFVAGQDTTSHALTFCLYLLAKNQDIQKKAREEILNVFGDEPKDVFPTLEDC |
| | L188I, D189E) | KKLNYLDMVIKESMRIYPPANDVLARDVNEDLNVKGVFIPKGSMVSVDIHAL |
| | | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 239 | Lichtheimia | ATGACAGAGATCAAGGAGCACATCTACAGATACCGTCACTATATCGGGaTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_64 | TTCCCTTTGCTATCTAAGTGTAATGGGGTATACCTTAATAGAATGCCATTT |
| | (V17I, A59D, | AAGTGGACAATTTATGTCGCGGATCCAGAAATCGCGAGGGCGATTCTTTTa |
| | F102L, D181E, | AAACCCGAATTCGGCCACAAGACCAGGAGTGTGACAGATTCCTTGGATAAG |
| | L188I, D189E) | AACTCAAGCCTATTTGAATTTGTTGGGGACGATAATATAGCCATAGTTAAT |
| | | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACAGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTTTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 240 | Lichtheimia | MTEIKEHIYRYRHYIGIAAAVALVCQQVYYRIFRIPKNLRHIPAIPYGQQLKAL |
| | ramose species | RSDEDLTSRTKRLVFPLLSKCNGVYLNRMPFKWTIYVADPEIARAILLKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_65 | WVKTYRQVFDSFTDVFSLVFPRLDPIYRYFSAKHREQYNAVYKLIDLLDGMA |
| | (V17I, A59D, | DKKRSMLQDASNSDIKDVPDHEKDLLQLMLEAELRGEGSWTKRELRHNMA |
| | F102L, D181E, | IFFVAGQDTTSHAMTFCLYLLAKNQDIQKKAREEILNVFGDEPKDVFPTLED |
| | L188I, D189E, | CKKLNYLDMVIKESMRIYPPANDVLARDVNEDLNVKGVFIPKGSMVSVDIH |
| | S208C, L325M) | ALHHRPDLWHEPDKFNPDRFLPGGEHDSHVGVTYAPFSSGSRQCIALKFAT |
| | | MQQRVVLSMLLRKYEWELPKDSKHKDSIQFQIPFNIAPKDLELTFHKRY* |
| 241 | Lichtheimia | ATGACAGAGATCAAGGAGCACATCTACAGATACCGTCACTATATCGGGaTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATGAGGaTCTTACCAGCAGGACGAAGCGTTTAGTA |
| | 14HC_P450_65 | TTCCCTTTGCTATCTAAGTGTAATGGGGTATACCTTAATAGAATGCCATTT |
| | (V17I, A59D, | AAGTGGACAATTTATGTCGCGGATCCAGAAATCGCGAGGGCGATTCTTTTa |
| | F102L, D181E, | AAACCCGAATTCGGCCACAAGACCAGGAGTGTGACAGATTCCTTGGATAAG |
| | L188I, D189E, | AACTCAAGCCTATTTGAATTTGTTGGGGACGATAATATAGCCATAGTTAAT |
| | S208C, L325M) | GGCCATGAATGGAAAGAGCAACGTAAGATAATGAACCCAGCATTCCACAGG |
| | | GCTACGCCCGTAGGGATGTTCGGCAGTTTAATGCCTAAAGTTTTTAGGCTT |
| | | GTGGAAGAACAGCCTACACTACCCGCCCTAGAaTTAATGCAAAAGTTAACG |
| | | aTAGAgGCACTTGGGAAGTCAGTATTCGGCTTTGAATTCGGCGCACTAGAT |
| | | GACCCCGACtGTGTGTGGGTTAAGACTTATAGACAAGTGTTTGACTCTTTT |
| | | ACCGACGTTTTCTCTCTAGTGTTTCCCCGTTTAGATCCAATCTACCGTTATT |
| | | TTTCTGCGAAACACAGAGAACAGTACAACGCTGTTTACAAACTAATTGATC |
| | | TGCTGGATGGAATGGCAGACAAGAAAAGGAGCATGTTGCAAGATGCCTCCA |
| | | ACTCCGATATCAAAGATGTTCCAGACCACGAGAAAGATTTACTGCAACTTA |
| | | TGTTGGAGGCTGAACTGCGTGGCGAAGGCTCATGGACAAAACGTGAATTGC |
| | | GTCATAACATGGCCATATTTTTTGTGGCAGGTCAGGACACAACGAGTCATG |
| | | CTaTGACTTTTTGCCTTTATTTACTTGCGAAGAATCAAGATATCCAGAAGA |
| | | AGGCGAGAGAAGAAATCTTAAATGTTTTTGGGGATGAACCTAAAGACGTG |
| | | TTTCCCACACTTGAAGATTGCAAAAAACTAAATTATCTTGACATGGTGATC |
| | | AAGGAATCCATGAGAATTTACCCTCCTGCTAACGATGTCCTAGCCAGGGAT |
| | | GTCAATGAAGATTTAAACGTCAAGGGTGTCTTCATTCCCAAGGGATCTATG |
| | | GTATCAGTGGACATTCATGCCCTACACCATAGACCAGACCTGTGGCATGAG |
| | | CCCGATAAGTTCAATCCCGATCGTTTCCTGCCGGGTGGGGAGCATGACAGT |
| | | CACGTCGGCGTAACATACGCCCCCTTCTCATCCGGCTCCAGACAGTGCATCG |
| | | CGTTAAAATTCGCTACCATGCAGCAAAGGGTAGTCCTGTCCATGTTGCTGA |
| | | GAAAGTACGAATGGGAATTGCCAAAGGATTCCAAACATAAGGACTCCATCC |
| | | AGTTCCAGATTCCCTTCAACATCGCTCCTAAGGATCTAGAGCTTACCTTTCA |
| | | TAAACGTTACTAG |
| 242 | Lichtheimia | MTEIKEHIYRYRHYIGVAAAVALVCQQVYYRIFRIPKNLRHIPAIPYGQQLKA |
| | ramose species | LRSDKDLTSRTKRLVFPLLSKCNGVYLNRMPFKWTIYVADPEIARAILLKPE |
| | of origin; amino | FGHKTRSVTDSLDKNSSLFEFVGDDNIAIVNGHEWKEQRKIMNPAFHRATP |
| | acid sequence; | VGMFGSLMPKVFRLVEEQPTLPALELMQKLTIEALGKSVFGFEFGALDDPDC |
| | 14HC_P450_66 | VWVKTYRQVFDSFTDVFSLVFPRLDPIYRYFSAKHREQYNAVYKLIDLLDGM |
| | (E58K, A59D, | ADKKRSMLQDASNSDIKDVPDHEKDLLQLMLEAELRGEGSWTKRELRHNM |
| | F102L, D181E, | AIFFVAGQDTTSHAMTFCLYLLAKNQDIQKKAREEILNVFGDEPKDVFPTLE |
| | L188I, D189E, | DCKKLNYLDMVIKESMRIYPPANDVLARDVNEDLNVKGVFIPKGSMVSVDI |
| | S208C, L325M) | HALHHRPDLWHEPDKFNPDRFLPGGEHDSHVGVTYAPFSSGSRQCIALKFA |
| | | TMQQRVVLSMLLRKYEWELPKDSKHKDSIQFQIPFNIAPKDLELTFHKRY* |
| 243 | Lichtheimia | ATGACAGAGATCAAGGAGCACATCTACAGATACCGTCACTATATCGGGGTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATaagGaTCTTACCAGCAGGACGAAGCGTTTAGTAT |
| | 14HC_P450_66 | TCCCTTTGCTATCTAAGTGTAATGGGGTATACCTTAATAGAATGCCATTTA |
| | (E58K, A59D, | AGTGGACAATTTATGTCGCGGATCCAGAAATCGCGAGGGCGATTCTTTTaA |
| | F102L, D181E, | AACCCGAATTCGGCCACAAGACCAGGAGTGTGACAGATTCCTTGGATAAGA |
| | L188I, D189E, | ACTCAAGCCTATTTGAATTTGTTGGGGACGATAATATAGCCATAGTTAATG |
| | S208C, L325M) | GCCATGAATGGAAAGAGCAACGTAAGATAATGAACCCAGCATTCCACAGGG |
| | | CTACGCCCGTAGGGATGTTCGGCAGTTTAATGCCTAAAGTTTTTAGGCTTG |
| | | TGGAAGAACAGCCTACACTACCCGCCCTAGAaTTAATGCAAAAGTTAACGa |
| | | TAGAgGCACTTGGGAAGTCAGTATTCGGCTTTGAATTCGGCGCACTAGATG |
| | | ACCCCGACtGTGTGTGGGTTAAGACTTATAGACAAGTGTTTGACTCTTTTAC |
| | | CGACGTTTTCTCTCTAGTGTTTCCCCGTTTAGATCCAATCTACCGTTATTTT |
| | | TCTGCGAAACACAGAGAACAGTACAACGCTGTTTACAAACTAATTGATCTG |
| | | CTGGATGGAATGGCAGACAAGAAAAGGAGCATGTTGCAAGATGCCTCCAAC |
| | | TCCGATATCAAAGATGTTCCAGACCACGAGAAAGATTTACTGCAACTTATG |
| | | TTGGAGGCTGAACTGCGTGGCGAAGGCTCATGGACAAAACGTGAATTGCGT |
| | | CATAACATGGCCATATTTTTTGTGGCAGGTCAGGACACAACGAGTCATGCT |
| | | aTGACTTTTTGCCTTTATTTACTTGCGAAGAATCAAGATATCCAGAAGAAG |
| | | GCGAGAGAAGAAATCTTAAATGTTTTTGGGGATGAACCTAAAGACGTGTTT |
| | | CCCACACTTGAAGATTGCAAAAAACTAAATTATCTTGACATGGTGATCAAG |
| | | GAATCCATGAGAATTTACCCTCCTGCTAACGATGTCCTAGCCAGGGATGTC |
| | | AATGAAGATTTAAACGTCAAGGGTGTCTTCATTCCCAAGGGATCTATGGTA |
| | | TCAGTGGACATTCATGCCCTACACCATAGACCAGACCTGTGGCATGAGCCC |
| | | GATAAGTTCAATCCCGATCGTTTCCTGCCGGGTGGGGAGCATGACAGTCAC |
| | | GTCGGCGTAACATACGCCCCCTTCTCATCCGGCTCCAGACAGTGCATCGCGT |
| | | TAAAATTCGCTACCATGCAGCAAAGGGTAGTCCTGTCCATGTTGCTGAGAA |
| | | AGTACGAATGGGAATTGCCAAAGGATTCCAAACATAAGGACTCCATCCAGT |
| | | TCCAGATTCCCTTCAACATCGCTCCTAAGGATCTAGAGCTTACCTTTCATA |
| | | AACGTTACTAG |
| 244 | Lichtheimia | MTEIKEHIYRYRHYIGIAAAVALVCQQVYYRIFRIPKNLRHIPAIPYGQQLKAL |
| | ramose species | RSDKDLTSRTKRLVFPLLSKCNGVYLNRMPFKWTIYVADPEIARAILLKPEF |
| | of origin; amino | GHKTRSVTDSLDKNSSLFEFVGDDNIAIVNGHEWKEQRKIMNPAFHRATPV |
| | acid sequence; | GMFGSLMPKVFRLVEEQPTLPALELMQKLTIEALGKSVFGFEFGALDDPDCV |
| | 14HC_P450_67 | WVKTYRQVFDSFTDVFSLVFPRLDPIYRYFSAKHREQYNAVYKLIDLLDGMA |
| | (V17I, E58K, | DKKRSMLQDASNSDIKDVPDHEKDLLQLMLEAELRGEGSWTKRELRHNMA |
| | A59D, F102L, | IFFVAGQDTTSHALTFCLYLLAKNQDIQKKAREEILNVFGDEPKDVFPTLEDC |
| | D181E, L188I, | KKLNYLDMVIKESMRIYPPANDVLARDVNEDLNVKGVFIPKGSMVSVDIHAL |
| | D189E, S208C) | HHRPDLWHEPDKFNPDRFLPGGEHDSHVGVTYAPFSSGSRQCIALKFATMQ |
| | | QRVVLSMLLRKYEWELPKDSKHKDSIQFQIPFNIAPKDLELTFHKRY* |
| 245 | Lichtheimia | ATGACAGAGATCAAGGAGCACATCTACAGATACCGTCACTATATCGGGaTT |
| | ramose species | GCGGCTGCTGTCGCTTTGGTGTGCCAACAAGTGTATTACAGGATCTTTCGT |
| | of origin; nucleic | ATCCCTAAGAACCTGAGGCACATACCTGCCATTCCGTACGGTCAACAACTA |
| | acid sequence; | AAGGCATTGAGGTCCGATaagGaTCTTACCAGCAGGACGAAGCGTTTAGTAT |
| | 14HC_P450_67 | TCCCTTTGCTATCTAAGTGTAATGGGGTATACCTTAATAGAATGCCATTTA |
| | (V17I, E58K, | AGTGGACAATTTATGTCGCGGATCCAGAAATCGCGAGGGCGATTCTTTTaA |
| | A59D, F102L, | AACCCGAATTCGGCCACAAGACCAGGAGTGTGACAGATTCCTTGGATAAGA |
| | D181E, L188I, | ACTCAAGCCTATTTGAATTTGTTGGGGACGATAATATAGCCATAGTTAATG |
| | D189E, S208C) | GCCATGAATGGAAAGAGCAACGTAAGATAATGAACCCAGCATTCCACAGGG |
| | | CTACGCCCGTAGGGATGTTCGGCAGTTTAATGCCTAAAGTTTTTAGGCTTG |
| | | TGGAAGAACAGCCTACACTACCCGCCCTAGAaTTAATGCAAAAGTTAACGa |
| | | TAGAgGCACTTGGGAAGTCAGTATTCGGCTTTGAATTCGGCGCACTAGATG |
| | | ACCCCGACtGTGTGTGGGTTAAGACTTATAGACAAGTGTTTGACTCTTTTAC |
| | | CGACGTTTTCTCTCTAGTGTTTCCCCGTTTAGATCCAATCTACCGTTATTTT |
| | | TCTGCGAAACACAGAGAACAGTACAACGCTGTTTACAAACTAATTGATCTG |
| | | CTGGATGGAATGGCAGACAAGAAAAGGAGCATGTTGCAAGATGCCTCCAAC |
| | | TCCGATATCAAAGATGTTCCAGACCACGAGAAAGATTTACTGCAACTTATG |
| | | TTGGAGGCTGAACTGCGTGGCGAAGGCTCATGGACAAAACGTGAATTGCGT |
| | | CATAACATGGCCATATTTTTTGTGGCAGGTCAGGACACAACGAGTCATGCT |
| | | TTGACTTTTTGCCTTTATTTACTTGCGAAGAATCAAGATATCCAGAAGAAG |
| | | GCGAGAGAAGAAATCTTAAATGTTTTTGGGGATGAACCTAAAGACGTGTTT |
| | | CCCACACTTGAAGATTGCAAAAAACTAAATTATCTTGACATGGTGATCAAG |
| | | GAATCCATGAGAATTTACCCTCCTGCTAACGATGTCCTAGCCAGGGATGTC |
| | | AATGAAGATTTAAACGTCAAGGGTGTCTTCATTCCCAAGGGATCTATGGTA |
| | | TCAGTGGACATTCATGCCCTACACCATAGACCAGACCTGTGGCATGAGCCC |
| | | GATAAGTTCAATCCCGATCGTTTCCTGCCGGGTGGGGAGCATGACAGTCAC |
| | | GTCGGCGTAACATACGCCCCCTTCTCATCCGGCTCCAGACAGTGCATCGCGT |
| | | TAAAATTCGCTACCATGCAGCAAAGGGTAGTCCTGTCCATGTTGCTGAGAA |
| | | AGTACGAATGGGAATTGCCAAAGGATTCCAAACATAAGGACTCCATCCAGT |
| | | TCCAGATTCCCTTCAACATCGCTCCTAAGGATCTAGAGCTTACCTTTCATA |
| | | AACGTTACTAG |

TABLE 18
Cytochrome P450 Reductase (CPR) sequences.

| SEQ | | |
|---|---|---|
| ID | | |
| NO. | Description | Sequence |
| 168 | Lichtheimia | MAQSPPALDTLDIVFLGTIGLGTIAWFARRQIAERLFGSTSSSDAKSNGHATP |
| | ramosa species | APPKRERNFVKIMQEQGRKVIFFYGSQTGTAEDYASRLAKECSQKYGVNCMT |
| | of origin; amino | ADLELYDLSYLDTVPEDCLVFFVMATYGEGEPTDNAVDFWDVLSEEEPQFSE |
| | acid sequence; | AEGDKPLQNLRYLVFGLGNKTYEHYNAVARNVDKRLEVLGAHRIHERGEGD |
| | 14HC_CPR_1 | DDGSLEEDFLAWQENMWPAFCEALGVDESNAHSGPRQATYSVEELVDVNM |
| | | DDVYLGELAEKPKEGARVIYDAKRPFNAPIAISQDLFTNTDRHCLHMEIDISD |
| | | SNLSYQTGDHIAIWPTNSENEVARLASILGLADKLDTAINVKAIDPAASKKYPF |
| | | PCPATYRAIFRHYLDICAAVSRQSLMAFVEYAPTEESKERLRQLAKDKDEYRL |
| | | TVGEAVRNLGEVLEIVAGNDVKPGFFSSVPFDLVVESISRLQPRYYSISSSAKES |
| | | PKKIAVTAVTLSYQPDPTPQRTVYGVNTNYLWRIHTASKQQSTEADLPTYDL |
| | | SGPRNALHGTKLPVHVRRSQFKLPRNPTVPVIMVGPGTGVAPFRGFVRERAL |
| | | QKSEGKPVGPTLLFFGCRNSEQDFLYKDEWPALFDTLGESSRIITAFSRETAQ |
| | | KVYVQHRLQENGQEVWGLLQRGAYIYVCGDAKNMARDVQQTFVNFGIEFGG |
| | | LSDDKAHDFVKNLRNTGRYQEDVWS* |
| 169 | Lichtheimia | ATGGCTCAATCTCCACCAGCATTGGATACTTTGGATATCGTTTTCTTGGGTA |
| | ramosa species | CTATCGGTTTGGGTACAATTGCTTGGTTTGCTAGAAGGCAAATTGCTGAAAG |
| | of origin; | ATTATTCGGTTCCACCTCTTCCTCTGATGCTAAATCTAATGGTCATGCTACT |
| | nucleic acid | CCAGCTCCACCAAAAAGAGAAAGAAACTTCGTTAAGATCATGCAAGAGCAA |
| | sequence; | GGTAGAAAGGTCATTTTCTTCTACGGTTCTCAAACTGGTACTGCTGAAGATT |
| | 14HC_CPR_1 | ACGCTTCTAGATTGGCTAAAGAATGCTCTCAAAAGTACGGTGTTAACTGTAT |
| | | GACTGCTGATTTGGAGTTGTACGACTTGTCTTACTTGGATACTGTTCCAGAA |
| | | GATTGCCTGGTTTTTTTCGTTATGGCTACTTATGGTGAAGGTGAACCTACTG |
| | | ATAATGCTGTTGATTTCTGGGATGTCTTGTCTGAAGAAGAACCACAATTTTC |
| | | TGAAGCCGAAGGTGATAAGCCATTGCAAAATTTGAGATACTTGGTTTTCGGC |
| | | TTGGGTAACAAGACTTACGAACATTATAACGCTGTTGCCAGAAACGTTGACA |
| | | AGAGATTGGAAGTTTTGGGTGCTCATAGAATTCATGAACGTGGTGAGGGTG |
| | | ATGATGATGGTTCTTTGGAAGAAGATTTTTTGGCCTGGCAAGAAAACATGT |
| | | GGCCAGCTTTTTGTGAAGCTTTGGGTGTTGATGAATCTAACGCTCATTCTGG |
| | | TCCAAGACAAGCTACTTATTCTGTTGAAGAATTGGTCGATGTCAACATGGAT |
| | | GATGTTTACTTGGGTGAATTGGCCGAAAAACCTAAAGAAGGTGCTAGAGTT |
| | | ATCTACGATGCTAAGAGGCCTTTTAATGCTCCAATTGCCATTTCTCAAGACT |
| | | TGTTCACTAACACCGATAGACATTGCTTGCACATGGAAATCGATATCTCCGA |
| | | TTCCAACTTGTCATACCAAACCGGTGATCATATTGCTATTTGGCCAACTAAC |
| | | TCCGAAAACGAAGTTGCTAGACTGGCTTCTATTTTGGGTTTAGCTGATAAGT |
| | | TGGATACCGCCATCAATGTTAAGGCTATTGATCCAGCTGCTTCCAAAAAGTA |
| | | CCCATTTCCTTGTCCAGCTACTTACAGAGCTATTTTCAGACATTACTTGGAC |
| | | ATTTGCGCTGCCGTTTCTAGACAATCTTTGATGGCTTTTGTTGAATACGCTC |
| | | CAACCGAAGAATCCAAAGAAAGATTGAGACAATTGGCCAAGGATAAGGATG |
| | | AGTACAGATTGACTGTTGGTGAAGCTGTTAGAAATTTGGGTGAAGTATTGG |
| | | AAATCGTTGCTGGTAATGATGTTAAGCCAGGTTTCTTTTCTTCCGTTCCATT |
| | | CGATTTGGTTGTCGAATCTATTTCCAGATTGCAACCTAGGTACTACTCCATT |
| | | TCTTCATCTGCAAAAGAGTCCCCAAAAAAGATCGCTGTTACTGCTGTTACAT |
| | | TGTCCTATCAACCAGATCCAACTCCACAAAGAACTGTTTATGGTGTCAACAC |
| | | TAATTACTTGTGGCGTATTCATACCGCCTCTAAGCAACAATCTACTGAAGCT |
| | | GATTTGCCAACCTATGATTTGTCAGGTCCAAGAAATGCTTTACATGGTACTA |
| | | AGTTGCCAGTTCACGTTAGAAGATCTCAATTCAAGTTGCCAAGAAACCCAAC |
| | | CGTTCCAGTTATTATGGTTGGTCCAGGTACTGGTGTTGCTCCTTTTAGAGGT |
| | | TTTGTTAGAGAAAGAGCCTTGCAGAAATCTGAAGGTAAACCAGTTGGTCCA |
| | | ACTTTGTTGTTTTTCGGTTGTAGAAACTCCGAACAGGACTTCTTGTACAAAG |
| | | ATGAATGGCCTGCTTTGTTTGACACCTTAGGTGAATCCTCTAGAATTATTAC |
| | | CGCCTTCTCAAGAGAAACCGCTCAAAAAGTTTACGTCCAGCATAGATTGCAA |
| | | GAGAACGGTCAAGAAGTTTGGGGTTTGTTGCAAAGGGGTGCTTATATCTAT |
| | | GTTTGTGGTGATGCAAAGAACATGGCCAGAGATGTTCAACAAACTTTCGTTA |
| | | ACTTCGGTATCGAATTCGGTGGTTTGTCTGATGATAAGGCTCATGATTTTGT |
| | | CAAGAACTTGAGAAACACCGGTAGATACCAAGAAGATGTTTGGTCTTGA |
| 170 | Thamnostylum | MSKKPTTFAISPLDLLLLGTLGIGALLYITRKLRAAPPPAAGDAARPSDKAPSN |
| | piriforme | KPERNFVKLMEQQKRRVIFFYGSQTGTAEDYASRLAKESSQKYGVSSMAADIE |
| | species of | LYDLSYLDTVPEDKLVVFVMATYGEGEPTDNAVDFWETLTDEAPMFSLGEEL |
| | origin; amino | APLRNLNYIVFGLGNKTYEHYNEVARVLDKRLAALGATRHGVRGEGDDDASL |
| | acid sequence; | EEDFLAWQEDMWPAFCAALNVDESNAQAGPRQAMYGVKELEGHDENGLFL |
| | 14HC_CPR_2 | GELGEWLQPEQLQKQTAVHDAKHPYLAPIAASRDLFSHAADRHCLHLEVDIA |
| | | SSNLTYQTGDHIAIWPTNNDVEVLRLATVLGLADKLDTAIMVKALDSAASKK |
| | | YPFPVPTTYRAAFKHYLDICSPASRQTLISLVDYAPTASSKDALHKLATDKDA |
| | | YKTQVSEAARNLAEVLELCQDDEGTTAAPGFFSTVPFDLIVESVSRLQPRYYSI |
| | | SSSSLTSPKSVAVTAVTLSYQPSTTKSRTVYGVNTNYLWRIHAPHDASVPAYD |
| | | LDGPRNTLQGKKVPVHIRRSTFKLPRNTKLPVIMVGPGTGVAPFRGFIHDRV |
| | | HQKQSGKEVGPTVLFYGCRHSKQDFLYAEEWPALFEALGEGSQLITAFSRESA |
| | | QKVYVQHRLKEHGEQMWKYIEQGAYIYVCGDAKNMARDVQQTFIEFAQALG |
| | | NKTESQAQDYVKNLRNTGRYQEDVWS* |
| 171 | Thamnostylum | ATGTCTAAAAAACCAACTACTTTTGCTATTTCTCCATTGGATTTGTTGTTGT |
| | piriforme | TGGGTACTTTGGGTATTGGTGCTTTGTTGTATATTACTAGAAAATTGAGAG |
| | species of | CTGCTCCACCACCAGCTGCTGGTGATGCTGCTAGACCATCTGATAAAGCTCC |
| | origin; nucleic | ATCTAATAAACCAGAAAGAAATTTTGTTAAATTGATGGAACAACAAAAAAG |
| | acid sequence; | AAGAGTTATTTTTTTTTATGGTTCTCAAACTGGTACTGCTGAAGATTATGCT |
| | 14HC_CPR_2 | TCTAGATTGGCTAAAGAATCTTCTCAAAAATATGGTGTTTCTTCTATGGCTG |
| | | CTGATATTGAATTGTATGATTTGTCTTATTTGGATACTGTTCCAGAAGATAA |
| | | ATTGGTTGTTTTTGTTATGGCTACTTATGGTGAAGGTGAACCAACTGATAAT |
| | | GCTGTTGATTTTTGGGAAACTTTGACTGATGAAGCTCCAATGTTTTCTTTGG |
| | | GTGAAGAATTGGCTCCATTGAGAAATTTGAATTATATTGTTTTTGGTTTGG |
| | | GTAATAAAACTTATGAACATTATAATGAAGTTGCTAGAGTTTTGGATAAAA |
| | | GATTGGCTGCTTTGGGTGCTACTAGACATGGTGTTAGAGGTGAAGGTGATG |
| | | ATGATGCTTCTTTGGAAGAAGATTTTTTGGCTTGGCAAGAAGATATGTGGCC |
| | | AGCTTTTTGTGCTGCTTTGAATGTTGATGAATCTAATGCTCAAGCTGGTCCA |
| | | AGACAAGCTATGTATGGTGTTAAAGAATTGGAAGGTCATGATGAAAATGGT |
| | | TTGTTTTTGGGTGAATTGGGTGAATGGTTGCAACCAGAACAATTGCAAAAA |
| | | CAAACTGCTGTTCATGATGCTAAACATCCATATTTGGCTCCAATTGCTGCTT |
| | | CTAGAGATTTGTTTTCTCATGCTGCTGATAGACATTGTTTGCATTTGGAAGT |
| | | TGATATTGCTTCTTCTAATTTGACTTATCAAACTGGTGATCATATTGCTATT |
| | | TGGCCAACTAATAATGATGTTGAAGTTTTGAGATTGGCTACTGTTTTGGGTT |
| | | TGGCTGATAAATTGGATACTGCTATTATGGTTAAAGCATTGGATTCTGCTGC |
| | | TTCTAAAAAATATCCATTTCCAGTTCCAACTACTTATAGAGCTGCTTTTAAA |
| | | CATTATTTGGATATTTGTTCTCCAGCTTCTAGACAAACTTTGATTTCTTTGG |
| | | TTGATTATGCTCCAACTGCTTCTTCTAAAGATGCTTTGCATAAATTGGCTAC |
| | | TGATAAAGATGCTTATAAAACTCAAGTTTCTGAAGCTGCTAGAAATTTGGC |
| | | TGAAGTTTTGGAATTGTGTCAAGATGATGAAGGTACTACTGCTGCTCCAGGT |
| | | TTTTTTTCTACTGTTCCATTTGATTTGATTGTTGAATCTGTTTCTAGATTGC |
| | | AACCAAGATATTATTCTATTTCTTCTTCTTCTTTGACTTCTCCAAAATCTGT |
| | | TGCTGTTACTGCTGTTACTTTGTCTTATCAACCATCTACTACTAAATCTAGA |
| | | ACTGTTTATGGTGTTAATACTAATTATTTGTGGAGAATCCATGCTCCACATG |
| | | ATGCTTCTGTTCCAGCTTATGATTTGGATGGTCCAAGAAATACTTTGCAAGG |
| | | TAAAAAAGTTCCAGTTCATATTAGAAGATCTACTTTTAAATTGCCAAGAAA |
| | | TACTAAATTGCCAGTTATTATGGTTGGTCCAGGTACTGGTGTTGCTCCATTT |
| | | AGAGGTTTTATTCATGATAGAGTTCATCAAAAACAATCTGGTAAAGAAGTT |
| | | GGTCCAACTGTTTTGTTTTATGGTTGTAGACATTCTAAACAAGATTTTTTGT |
| | | ATGCTGAAGAATGGCCAGCTTTGTTTGAAGCATTGGGTGAAGGTTCTCAATT |
| | | GATTACTGCTTTTTCTAGAGAATCTGCTCAAAAAGTTTATGTTCAACATAGA |
| | | TTGAAAGAACATGGTGAACAAATGTGGAAATATATTGAACAAGGTGCTTAT |
| | | ATTTATGTTTGTGGTGATGCTAAAAATATGGCTAGAGATGTTCAACAAACT |
| | | TTTATTGAATTTGCTCAAGCATTGGGTAATAAAACTGAATCTCAAGCTCAAG |
| | | ATTATGTTAAAAATTTGAGAAATACTGGTAGATACCAAGAAGATGTTTGGT |
| | | CTTAG |
| 172 | Homo sapiens | MINMGDSHVDTSSTVSEAVAEEVSLFSMTDMILFSLIVGLLTYWFLFRKKKEE |
| | species of | VPEFTKIQTLTSSVRESSFVEKMKKTGRNIIVFYGSQTGTAEEFANRLSKDAHR |
| | origin; amino | YGMRGMSADPEEYDLADLSSLPEIDNALVVFCMATYGEGDPTDNAQDFYDW |
| | acid sequence; | LQETDVDLSGVKFAVFGLGNKTYEHFNAMGKYVDKRLEQLGAQRIFELGLGD |
| | 14HC_CPR_3 | DDGNLEEDFITWREQFWPAVCEHFGVEATGEESSIRQYELVVHTDIDAAKVY |
| | | MGEMGRLKSYENQKPPFDAKNPFLAAVTTNRKLNQGTERHLMHLELDISDS |
| | | KIRYESGDHVAVYPANDSALVNQLGKILGADLDVVMSLNNLDEESNKKHPFP |
| | | CPTSYRTALTYYLDITNPPRTNVLYELAQYASEPSEQELLRKMASSSGEGKELY |
| | | LSWVVEARRHILAILQDCPSLRPPIDHLCELLPRLQARYYSIASSSKVHPNSVHI |
| | | CAVVVEYETKAGRINKGVATNWLRAKEPAGENGGRALVPMFVRKSQFRLPF |
| | | KATTPVIMVGPGTGVAPFIGFIQERAWLRQQGKEVGETLLYYGCRRSDEDYLY |
| | | REELAQFHRDGALTQLNVAFSREQSHKVYVQHLLKQDREHLWKLIEGGAHIY |
| | | VCGDARNMARDVQNTFYDIVAELGAMEHAQAVDYIKKLMTKGRYSLDVWS |
| | | RRAMAEQSDEAVKYYTLEEIQKHNHSKSTWLILHHKVYDLTKFLEEHPGGEE |
| | | VLREQAGGDATENFEDVGHSTDAREMSKTFIIGELHPDDRPKLNKPPETLITT |
| | | ID* |
| 173 | Homo sapiens | ATGATCAACATGGGTGATTCTCACGTTGATACCTCTTCTACTGTTTCTGAAG |
| | species of | CTGTTGCTGAAGAAGTCTCTTTGTTCTCTATGACCGATATGATCCTGTTCTC |
| | origin; nucleic | TTTGATCGTTGGTTTGTTGACTTACTGGTTCCTGTTCAGAAAGAAGAAAGAG |
| | acid sequence; | GAAGTTCCAGAGTTCACCAAGATTCAAACTTTGACCTCTTCCGTCAGAGAAT |
| | 14HC_CPR_3 | CTTCATTCGTTGAGAAGATGAAGAAAACCGGTAGAAACATCATCGTGTTCTA |
| | | CGGTTCTCAAACTGGTACAGCTGAAGAATTTGCTAACAGATTGTCTAAGGAT |
| | | GCCCATAGATATGGTATGAGAGGTATGTCTGCTGATCCAGAAGAATACGATT |
| | | TGGCTGATTTGTCATCCTTGCCAGAAATTGATAATGCCTTGGTTGTTTTCTG |
| | | TATGGCTACTTATGGTGAAGGTGATCCAACTGATAATGCTCAAGATTTCTAC |
| | | GACTGGTTGCAAGAAACTGATGTTGATTTGTCTGGTGTTAAGTTCGCTGTTT |
| | | TCGGTTTGGGTAACAAGACTTACGAACATTTTAACGCTATGGGCAAGTACGT |
| | | TGACAAGAGATTGGAACAATTGGGTGCCCAAAGAATCTTCGAATTAGGTTT |
| | | GGGTGATGATGACGGTAATTTGGAAGAAGATTTCATCACTTGGAGAGAACA |
| | | ATTTTGGCCAGCTGTTTGTGAACATTTTGGTGTTGAAGCTACTGGTGAGGAA |
| | | TCCTCTATTAGACAATACGAATTGGTTGTTCACACCGATATTGATGCTGCTA |
| | | AAGTCTATATGGGTGAGATGGGTAGATTGAAGTCTTACGAAAATCAAAAGC |
| | | CACCATTCGATGCTAAGAATCCATTCTTGGCTGCTGTTACTACCAACAGAAA |
| | | ATTGAATCAGGGTACTGAAAGACACTTGATGCACTTGGAATTGGATATCTCC |
| | | GATTCTAAGATCAGGTACGAATCTGGTGATCATGTTGCTGTTTATCCAGCTA |
| | | ATGATTCCGCCTTGGTTAATCAATTGGGTAAGATTTTGGGTGCCGATTTGGA |
| | | TGTTGTTATGTCCTTGAACAACTTGGACGAAGAGTCCAACAAGAAACATCCA |
| | | TTTCCATGTCCAACCTCTTACAGAACTGCTTTGACTTATTACCTGGACATTA |
| | | CCAATCCACCAAGAACTAATGTCTTGTACGAATTAGCTCAATACGCCTCTGA |
| | | ACCATCCGAACAAGAATTATTGAGAAAGATGGCTTCCTCATCCGGTGAAGGT |
| | | AAAGAATTATACTTGTCCTGGGTTGTTGAAGCCAGAAGGCATATTTTGGCTA |
| | | TATTGCAAGATTGCCCATCCTTAAGACCACCAATTGATCATTTGTGCGAACT |
| | | GTTGCCAAGATTGCAAGCTAGATATTACTCCATTGCCTCCTCATCTAAGGTT |
| | | CATCCAAACTCTGTTCATATTTGCGCCGTTGTTGTTGAATACGAAACAAAAG |
| | | CTGGTAGGATTAACAAGGGTGTTGCTACTAATTGGTTGAGAGCTAAAGAAC |
| | | CTGCTGGTGAAAATGGTGGTAGAGCTTTGGTTCCAATGTTCGTTAGAAAGTC |
| | | CCAATTCAGATTGCCATTCAAAGCTACTACCCCAGTTATTATGGTTGGTCCA |
| | | GGTACTGGTGTTGCTCCTTTTATTGGTTTCATTCAAGAAAGAGCCTGGTTGA |
| | | GACAACAGGGTAAAGAAGTTGGAGAAACCTTGTTGTATTACGGTTGTAGAA |
| | | GATCCGACGAGGATTACTTGTATAGAGAAGAATTGGCTCAGTTCCATAGAG |
| | | ATGGTGCTTTGACACAATTGAACGTTGCCTTTTCTAGAGAACAGTCCCATAA |
| | | GGTTTACGTCCAACACTTGTTGAAACAAGACAGAGAACATCTGTGGAAGTTG |
| | | ATTGAAGGTGGTGCTCATATCTACGTTTGTGGTGATGCTAGAAATATGGCTA |
| | | GAGATGTTCAAAACACCTTCTACGATATTGTCGCTGAATTAGGTGCTATGGA |
| | | ACATGCTCAAGCTGTTGACTACATCAAGAAACTAATGACCAAGGGCAGATAC |
| | | TCATTGGATGTTTGGTCTAGAAGAGCTATGGCCGAACAATCTGATGAAGCTG |
| | | TTAAGTACTACACCTTGGAAGAAATCCAAAAGCACAACCATTCTAAGTCTAC |
| | | CTGGTTGATCTTGCATCACAAGGTTTACGATTTGACCAAGTTCTTAGAAGAA |
| | | CATCCAGGTGGTGAAGAAGTCTTGAGAGAACAAGCTGGTGGTGATGCTACT |
| | | GAAAATTTTGAAGATGTTGGTCATTCCACCGATGCCAGAGAAATGTCTAAG |
| | | ACTTTCATTATCGGTGAATTGCACCCAGATGATAGACCAAAATTGAACAAAC |
| | | CACCAGAAACCTTGATTACCACCATCGATTAA |
| 174 | Arabidopsis | MTSALYASDLFKQLKSIMGTDSLSDDVVLVIATTSLALVAGFVVLLWKKTTAD |
| | thaliana species | RSGELKPLMIPKSLMAKDEDDDLDLGSGKTRVSIFFGTQTGTAEGFAKALSEE |
| | of origin; amino | IKARYEKAAVKVIDLDDYAADDDQYEEKLKKETLAFFCVATYGDGEPTDNAA |
| | acid sequence; | RFYKWFTEENERDIKLQQLAYGVFALGNRQYEHFNKIGIVLDEELCKKGAKR |
| | 14HC_CPR_4 | LIEVGLGDDDQSIEDDFNAWKESLWSELDKLLKDEDDKSVATPYTAVIPEYR |
| | | VVTHDPRFTTQKSMESNVANGNTTIDIHHPCRVDVAVQKELHTHESDRSCIH |
| | | LEFDISRTGITYETGDHVGVYAENHVEIVEEAGKLLGHSLDLVFSIHADKEDGS |
| | | PLESAVPPPFPGPCTLGTGLARYADLLNPPRKSALVALAAYATEPSEAEKLKH |
| | | LTSPDGKDEYSQWIVASQRSLLEVMAAFPSAKPPLGVFFAAIAPRLQPRYYSIS |
| | | SSPRLAPSRVHVTSALVYGPTPTGRIHKGVCSTWMKNAVPAEKSHECSGAPIF |
| | | IRASNFKLPSNPSTPIVMVGPGTGLAPFRGFLQERMALKEDGEELGSSLLFFGC |
| | | RNRQMDFIYEDELNNFVDQGVISELIMAFSREGAQKEYVQHKMMEKAAQVW |
| | | DLIKEEGYLYVCGDAKGMARDVHRTLHTIVQEQEGVSSSEAEAIVKKLQTEGR |
| | | YLRDVW* |
| 175 | Arabidopsis | ATGACTTCTGCTTTGTATGCTTCCGATTTGTTTAAGCAGCTCAAGTCAATTA |
| | thaliana species | TGGGGACAGATTCGTTATCCGACGATGTTGTACTTGTGATTGCAACGACGTC |
| | of origin; | TTTGGCACTAGTAGCTGGATTTGTGGTGTTGTTATGGAAGAAAACGACGGC |
| | nucleic acid | GGATCGGAGCGGGGAGCTGAAGCCTTTGATGATCCCTAAGTCTCTTATGGCT |
| | sequence; | AAGGACGAGGATGATGATTTGGATTTGGGATCCGGGAAGACTAGAGTCTCT |
| | 14HC_CPR_4 | ATCTTCTTCGGTACGCAGACTGGAACAGCTGAGGGATTTGCTAAGGCATTAT |
| | | CCGAAGAAATCAAAGCGAGATATGAAAAAGCAGCAGTCAAAGTCATTGACT |
| | | TGGATGACTATGCTGCCGATGATGACCAGTATGAAGAGAAATTGAAGAAGG |
| | | AAACTTTGGCATTTTTCTGTGTTGCTACTTATGGAGATGGAGAGCCTACTGA |
| | | CAATGCTGCCAGATTTTACAAATGGTTTACGGAGGAAAATGAACGGGATAT |
| | | AAAGCTTCAACAACTAGCATATGGTGTGTTTGCTCTTGGTAATCGCCAATAT |
| | | GAACATTTTAATAAGATCGGGATAGTTCTTGATGAAGAGTTATGTAAGAAA |
| | | GGTGCAAAGCGTCTTATTGAAGTCGGTCTAGGAGATGATGATCAGAGCATT |
| | | GAGGATGATTTTAATGCCTGGAAAGAATCACTATGGTCTGAGCTAGACAAG |
| | | CTCCTCAAAGACGAGGATGATAAAAGTGTGGCAACTCCTTATACAGCTGTTA |
| | | TTCCTGAATACCGGGTGGTGACTCATGATCCTCGGTTTACAACTCAAAAATC |
| | | AATGGAATCAAATGTGGCCAATGGAAATACTACTATTGACATTCATCATCCC |
| | | TGCAGAGTTGATGTTGCTGTGCAGAAGGAGCTTCACACACATGAATCTGATC |
| | | GGTCTTGCATTCATCTCGAGTTCGACATATCCAGGACGGGTATTACATATGA |
| | | AACAGGTGACCATGTAGGTGTATATGCTGAAAATCATGTTGAAATAGTTGA |
| | | AGAAGCTGGAAAATTGCTTGGCCACTCTTTAGATTTAGTATTTTCCATACAT |
| | | GCTGACAAGGAAGATGGCTCCCCATTGGAAAGCGCAGTGCCGCCTCCTTTCC |
| | | CTGGTCCATGCACACTTGGGACTGGTTTGGCAAGATACGCAGACCTTTTGAA |
| | | CCCTCCTCGAAAGTCTGCGTTAGTTGCCTTGGCGGCCTATGCCACTGAACCA |
| | | AGTGAAGCCGAGAAACTTAAGCACCTGACATCACCTGATGGAAAGGATGAG |
| | | TACTCACAATGGATTGTTGCAAGTCAGAGAAGTCTTTTAGAGGTGATGGCTG |
| | | CTTTTCCATCTGCAAAACCCCCACTAGGTGTATTTTTTGCTGCAATAGCTCC |
| | | TCGTCTACAACCTCGTTACTACTCCATCTCATCCTCGCCAAGATTGGCGCCAA |
| | | GTAGAGTTCATGTTACATCCGCACTAGTATATGGTCCAACTCCTACTGGTAG |
| | | AATCCACAAGGGTGTGTGTTCTACGTGGATGAAGAATGCAGTTCCTGCGGAG |
| | | AAAAGTCATGAATGTAGTGGAGCCCCAATCTTTATTCGAGCATCTAATTTCA |
| | | AGTTACCATCCAACCCTTCAACTCCAATCGTTATGGTGGGACCTGGGACTGG |
| | | GCTGGCACCTTTTAGAGGTTTTCTGCAGGAAAGGATGGCACTAAAAGAAGA |
| | | TGGAGAAGAACTAGGTTCATCTTTGCTCTTCTTTGGGTGTAGAAATCGACAG |
| | | ATGGACTTTATATACGAGGATGAGCTCAATAATTTTGTTGATCAAGGCGTA |
| | | ATATCTGAGCTCATCATGGCATTCTCCCGTGAAGGAGCTCAGAAGGAGTATG |
| | | TTCAACATAAGATGATGGAGAAGGCAGCACAAGTTTGGGATCTAATAAAGG |
| | | AAGAAGGATATCTCTATGTATGCGGTGATGCTAAGGGCATGGCGAGGGACG |
| | | TCCACCGAACTCTACACACCATTGTTCAGGAGCAGGAAGGTGTGAGTTCGTC |
| | | AGAGGCAGAGGCTATAGTTAAGAAACTTCAAACCGAAGGAAGATACCTCAG |
| | | AGATGTCTGGTGA |
| 248 | Thamnostylum | MSKKPTTFAISPLDLLLLGTLGIGALLYITRKLRAAPPPAAGDAARPSDKAPSN |
| | piriforme | KPERNFVKLMEQQKRRVIFFYGSQTGTAEDYASRLAKESSQKYGVSSMAADIE |
| | species of origin; | LYDLSYLDTVPEDKLVVFVMATYGEGEPTDNAVDFWETLTDEAPMFSLGEEL |
| | amino acid | APLRNLNYIVFGLGNKTYEHYNEVARVLDKRLAALGATRHGVRGEGDDDASL |
| | sequence; | EEDFLAWQEDMWPAFCAALNVDESNAQAGPRQAMYGVKELEGYDENGLFL |
| | 14HC_CPR_2 | GELGEWLQPEQLQKQTAVHDAKHPYLAPIAASRDLFSHAADRHCLHLEVDIA |
| | | SSNLTYQTGDHIAIWPTNNDVEVLRLATVLGLADKLDTAIMVKALDSAASKK |
| | | YPFPVPTTYRAAFKHYLDICSPASRQTLISLVDYAPTASSKDALHKLATDKDA |
| | | YKTQVSEAARNLAEVLELCQDDEGTTAAPGFFSTVPFDLIVESVSRLQPRYYSI |
| | | SSSSLTSPKSVAVTAVTLSYQPSTTKSRTVYGVNTNYLWRIHAPHDASVPAYD |
| | | LDGPRNTLQGKKVPVHIRRSTFKLPRNTKLPVIMVGPGTGVAPFRGFIHDRV |
| | | HQKQSGKEVGPTVLFYGCRHSKQDFLYAEEWPALFEALGEGSQLITAFSRESA |
| | | QKVYVQHRLKEHGEQMWKYIEQGAYIYVCGDAKNMARDVQQTFIEFAQALG |
| | | NKTESQAQDYVKNLRNTGRYQEDVWS* |
| 249 | Thamnostylum | atgtctaaaaaaccaactacttttgctatttctccattggatttgttgttgttgggtactttgggtattggtgct |
| | piriforme | ttgttgtatattactagaaaattgagagctgctccaccaccagctgctggtgatgctgctagaccatctgat |
| | species of origin; | aaagctccatctaataaaccagaaagaaattttgttaaattgatggaacaacaaaaaagaagagttattt |
| | nucleic acid | ttttttatggttctcaaactggtactgctgaagattatgcttctagattggctaaagaatcttctcaaaaata |
| | sequence; | tggtgtttcttctatggctgctgatattgaattgtatgatttgtcttatttggatactgttccagaagataaatt |
| | 14HC_CPR_2 | ggttgtttttgttatggctacttatggtgaaggtgaaccaactgataatgctgttgatttttgggaaactttg |
| | | actgatgaagctccaatgttttctttgggtgaagaattggctccattgagaaatttgaattatattgtttttg |
| | | gtttgggtaataaaacttatgaacattataatgaagttgctagagttttggataaaagattggctgctttgg |
| | | gtgctactagacatggtgttagaggtgaaggtgatgatgatgcttctttggaagaagattttttggcttggc |
| | | aagaagatatgtggccagctttttgtgctgctttgaatgttgatgaatctaatgctcaagctggtccaagac |
| | | aagctatgtatggtgttaaagaattggaaggtTatgatgaaaatggtttgtttttgggtgaattgggtgaa |
| | | tggttgcaaccagaacaattgcaaaaacaaactgctgttcatgatgctaaacatccatatttggctccaat |
| | | tgctgcttctagagatttgttttctcatgctgctgatagacattgtttgcatttggaagttgatattgcttcttc |
| | | taatttgacttatcaaactggtgatcatattgctatttggccaactaataatgatgttgaagttttgagattg |
| | | gctactgttttgggtttggctgataaattggatactgctattatggttaaagcattggattctgctgcttctaa |
| | | aaaatatccatttccagttccaactacttatagagctgcttttaaacattatttggatatttgttctccagctt |
| | | ctagacaaactttgatttctttggttgattatgctccaactgcttcttctaaagatgctttgcataaattggct |
| | | actgataaagatgcttataaaactcaagtttctgaagctgctagaaatttggctgaagttttggaattgtgt |
| | | caagatgatgaaggtactactgctgctccaggttttttttctactgttccatttgatttgattgttgaatctgtt |
| | | tctagattgcaaccaagatattattctatttcttcttcttctttgacttctccaaaatctgttgctgttactgctg |
| | | ttactttgtcttatcaaccatctactactaaatctagaactgtttatggtgttaatactaattatttgtggaga |
| | | atccatgctccacatgatgcttctgttccagcttatgatttggatggtccaagaaatactttgcaaggtaaa |
| | | aaagttccagttcatattagaagatctacttttaaattgccaagaaatactaaattgccagttattatggtt |
| | | ggtccaggtactggtgttgctccatttagaggttttattcatgatagagttcatcaaaaacaatctggtaaa |
| | | gaagttggtccaactgttttgttttatggttgtagacattctaaacaagattttttgtatgctgaagaatggc |
| | | cagctttgtttgaagcattgggtgaaggttctcaattgattactgctttttctagagaatctgctcaaaaagt |
| | | ttatgttcaacatagattgaaagaacatggtgaacaaatgtggaaatatattgaacaaggtgcttatattt |
| | | atgtttgtggtgatgctaaaaatatggctagagatgttcaacaaacttttattgaatttgctcaagcattgg |
| | | gtaataaaactgaatctcaagctcaagattatgttaaaaatttgagaaatactggtagataccaagaaga |
| | | tgtttggtcttag |
| 250 | Lichtheimia | MAQSPPALDTLDIVFLGTIGLGTIAWFARRQIAERLFGSTSSSDAKSNGHATP |
| | ramosa species | APPKRERNFVKIMQEQGRKVIFFYGSQTGTAEDYASRLAKECSQKYGVNCMT |
| | of origin; amino | ADLELYDLSYLDTVPEDCLVFFVMATYGEGEPTDNAVDFWDVLSEEEPQFSE |
| | acid sequence; | AEGDKPLQNLRYLVFGLGNKTYEHYNAVARNVDKRLEVLGAHRIHERGEGD |
| | 14HC_CPR_1 | DDGSLEEDFLAWQENMWPAFCEALGVDESNAHSGPRQATYSVEELVDVNM |
| | | DDVYLGELAEKPKEGARVIYDAKRPFNAPIAISQDLFTNTDRHCLHMEIDISD |
| | | SNLSYQTGDHIAIWPTNSENEVARLASILGLADKLDTAINVKAIDPAASKKYPF |
| | | PCPATYRAIFRHYLDICAAVSRQSLMAFVEYAPTEESKERLRQLAKDKDEYRL |
| | | TVGEAVRNLGEVLEIVAGNDVKPGFFSSVPFDLVVESISRLQPRYYSISSSAKES |
| | | PKKIAVTAVTLSYQPDPTPQRTVYGVNTNYLWRIHTASKQQSTEADLPTYDL |
| | | SGPRNALHGTKLPVHVRRSQFKLPRNPTVPVIMVGPGTGVAPFRGFVRERAL |
| | | QKSEGKPVGPTLLFFGCRNSEQDFLYKDEWPALFDTLGESSRIITAFSRETAQ |
| | | KVYVQHRLQENGQEVWGLLQRGAYIYVCGDAKNMARDVQQTFVNFGIEFGG |
| | | LSDDKAHDFVKNLRNTGRYQEDVWS* |
| 251 | Lichtheimia | atggctcaatctccaccagcattggatactttggatatcgttttcttgggtactatcggtttgggtacaattg |
| | ramosa species | cttggtttgctagaaggcaaattgctgaaagattattcggttccacctcttcctctgatgctaaatctaatgg |
| | of origin; nucleic | tcatgctactccagctccaccaaaaagagaaagaaacttcgttaagatcatgcaagagcaaggtagaaa |
| | acid sequence; | ggtcattttcttctacggttctcaaactggtactgctgaagattacgcttctagattggctaaagaatgctct |
| | 14HC_CPR_1 | caaaagtacggtgttaactgtatgactgctgatttggagttgtacgacttgtcttacttggatactgttccag |
| | | aagattgcctggtttttttcgttatggctacttatggtgaaggtgaacctactgataatgctgttgatttctgg |
| | | gatgtcttgtctgaagaagaaccacaattttctgaagccgaaggtgataagccattgcaaaatttgagat |
| | | acttggttttcggcttgggtaacaagacttacgaacattataacgctgttgccagaaacgttgacaagaga |
| | | ttggaagttttgggtgctcatagaattcatgaacgtggtgagggtgatgatgatggttctttggaagaaga |
| | | ttttttggcctggcaagaaaacatgtggccagctttttgtgaagctttgggtgttgatgaatctaacgctcat |
| | | tctggtccaagacaagctacttattctgttgaagaattggtcgatgtcaacatggatgatgtttacttgggt |
| | | gaattggccgaaaaacctaaagaaggtgctagagttatctacgatgctaagaggccttttaatgctccaa |
| | | ttgccatttctcaagacttgttcactaacaccgatagacattgcttgcacatggaaatcgatatctccgatt |
| | | ccaacttgtcataccaaaccggtgatcatattgctatttggccaactaactccgaaaacgaagttgctaga |
| | | ctggcttctattttgggtttagctgataagttggataccgccatcaatgttaaggctattgatccagctgctt |
| | | ccaaaaagtacccatttccttgtccagctacttacagagctattttcagacattacttggacatttgcgctgc |
| | | cgtttctagacaatctttgatggcttttgttgaatacgctccaaccgaagaatccaaagaaagattgagac |
| | | aattggccaaggataaggatgagtacagattgactgttggtgaagctgttagaaatttgggtgaagtatt |
| | | ggaaatcgttgctggtaatgatgttaagccaggtttcttttcttccgttccattcgatttggttgtcgaatcta |
| | | tttccagattgcaacctaggtactactccatttcttcatctgcaaaagagtccccaaaaaagatcgctgtta |
| | | ctgctgttacattgtcctatcaaccagatccaactccacaaagaactgtttatggtgtcaacactaattact |
| | | tgtggcgtattcataccgcctctaagcaacaatctactgaagctgatttgccaacctatgatttgtcaggtc |
| | | caagaaatgctttacatggtactaagttgccagttcacgttagaagatctcaattcaagttgccaagaaac |
| | | ccaaccgttccagttattatggttggtccaggtactggtgttgctccttttagaggttttgttagagaaagag |
| | | ccttgcagaaatctgaaggtaaaccagttggtccaactttgttgtttttcggttgtagaaactccgaacagg |
| | | acttcttgtacaaagatgaatggcctgctttgtttgacaccttaggtgaatcctctagaattattaccgcctt |
| | | ctcaagagaaaccgctcaaaaagtttacgtccagcatagattgcaagagaacggtcaagaagtttgggg |
| | | tttgttgcaaaggggtgcttatatctatgtttgtggtgatgcaaagaacatggccagagatgttcaacaaa |
| | | ctttcgttaacttcggtatcgaattcggtggtttgtctgatgataaggctcatgattttgtcaagaacttgag |
| | | aaacaccggtagataccaagaagatgtttggtcttga |
| 252 | Thamnostylum | MAQSSPALLDSLDIVFLGTIGLGTIAWFARRQIAERIFGSKDDANKNVGNGNA |
| | piriforme | PTAPKRERNFVKVMQEQGRKVIFFYGSQTGTAEDYASRLAKECSQKYGVSCM |
| | species of origin; | TADIELYDLTYLDTVPEDFLVFFIMATYGEGEPTDNAVDFWEQLTEEEPQFSE |
| | amino acid | GDTLGNLRYVVFGLGNKTYEHYNEVARRMDKLLTKLGAKRIGERGEGDDDA |
| | sequence; | SLEEDFLAWQDSMWPAFCDALGVDESNGSSGPRQAMYAVEELEGQEAVYLG |
| | 14HC_CPR_5 | ELGEKPKEGVKVVYDAKRPYNAPLVSQDLFKNTDRHCLHIDIDVSDSNLSYQT |
| | | GDHIAIWPTNSDDEVARLASLLGLTDKLDTSVMVKAIDSTASKQYPFPVPATY |
| | | RSIFRHYLDICAPASRQTLMSLVEYAPTEASKEALRLLSKDKDEYRLKVGEAV |
| | | RNLGEVLELAAGADARPGLFSTVPFDLIVESVSRLQPRYYSISSSAKESPKVIAV |
| | | TAVTLTYNPDPTPERTVYGVNTNYLWQIHAAKHNVNDGQRYPTYDLAGPRN |
| | | ALQGAKVPVHIRRSQFKLPRNPTVPVIMVGPGTGVAPFRGFVRERAAQKTDG |
| | | KPVGPTLLFFGCRNSQQDFLYKDEWPELFATLGDESRIVTAFSRETPQKVYVQ |
| | | HRLQENGEELWNLLQKGAYIYVCGDAKNMARDVNQTFVNFAIEFGGQTEEK |
| | | AHDYVKNLRNSGRYQEDVWS* |
| 253 | Thamnostylum | atggctcaatcttctccagctttgttggattctttggatattgtttttttgggtactattggtttgggtactattg |
| | piriforme | cttggtttgctagaagacaaattgctgaaagaatttttggttctaaagatgatgctaataaaaatgttggta |
| | species of origin; | atggtaatgctccaactgctccaaaaagagaaagaaattttgttaaagttatgcaagaacaaggtagaa |
| | nucleic acid | aagttatttttttttatggttctcaaactggtactgctgaagattatgcttctagattggctaaagaatgttct |
| | sequence; | caaaaatatggtgtttcttgtatgactgctgatattgaattgtatgatttgacttatttggatactgttccaga |
| | 14HC_CPR_5 | agattttttggttttttttattatggctacttatggtgaaggtgaaccaactgataatgctgttgatttttggg |
| | | aacaattgactgaagaagaaccacaattttctgaaggtgatactttgggtaatttgagatatgttgtttttg |
| | | gtttgggtaataaaacttatgaacattataatgaagttgctagaagaatggataaattgttgactaaattg |
| | | ggtgctaaaagaattggtgaaagaggtgaaggtgatgatgatgcttctttggaagaagattttttggcttg |
| | | gcaagattctatgtggccagctttttgtgatgctttgggtgttgatgaatctaatggttcttctggtccaaga |
| | | caagctatgtatgctgttgaagaattggaaggtcaagaagctgtttatttgggtgaattgggtgaaaaacc |
| | | aaaagaaggtgttaaagttgtttatgatgctaaaagaccatataatgctccattggtttctcaagatttgttt |
| | | aaaaatactgatagacattgtttgcatattgatattgatgtttctgattctaatttgtcttatcaaactggtga |
| | | tcatattgctatttggccaactaattctgatgatgaagttgctagattggcttctttgttgggtttgactgata |
| | | aattggatacttctgttatggttaaagctattgattctactgcttctaaacaatatccatttccagttccagct |
| | | acttatagatctatttttagacattatttggatatttgtgctccagcttctagacaaactttgatgtctttggtt |
| | | gaatatgctccaactgaagcatctaaagaagcattgagattgttgtctaaagataaagatgaatatagat |
| | | tgaaagttggtgaagctgttagaaatttgggtgaagttttggaattggctgctggtgctgatgctagacca |
| | | ggtttgttttctactgttccatttgatttgattgttgaatctgtttctagattgcaaccaagatattattctattt |
| | | cttcttctgctaaagaatctccaaaagttattgctgttactgctgttactttgacttataatccagatccaact |
| | | ccagaaagaactgtttatggtgttaatactaattatttgtggcaaattcatgctgctaaacataatgttaat |
| | | gatggtcaaagatacccaacttatgatttggctggtccaagaaatgctttgcaaggtgctaaagttccagt |
| | | tcatattagaagatctcaatttaaattgccaagaaatccaactgttccagttattatggttggtccaggtac |
| | | tggtgttgctccatttagaggttttgttagagaaagagctgctcaaaaaactgatggtaaaccagttggtc |
| | | caactttgttgttttttggttgtagaaattctcaacaagattttttgtataaagatgaatggccagaattgttt |
| | | gctactttgggtgatgaatctagaattgttactgctttttctagagaaactccacaaaaagtttatgttcaac |
| | | atagattgcaagaaaatggtgaagaattgtggaatttgttgcaaaaaggtgcttatatttatgtttgtggtg |
| | | atgctaaaaatatggctagagatgttaatcaaacttttgttaattttgctattgaatttggtggtcaaactga |
| | | agaaaaagctcatgattatgttaaaaatttgagaaattctggtagataccaagaagatgtttggtcttag |
| 254 | Saccharomyces | MPFGIDNTDFTVLAGLVLAVLLYVKRNSIKELLMSDDGDITAVSSGNRDIAQV |
| | cerevisiae and | VTENNKNYLVLYASQTGTAEDYAKKFSKELVAKFNLNVMCADVENYDFESL |
| | Homo sapiens | NDVPVIVSIFISTYGEGDFPDGAVNLEDFICNAEAGALSNLRYNMFGLGNSTYK |
| | species of origin; | FFNGAAKKAEKHLSAAGAIRLGKLGEADDGAGTTDEDYMAWKDSILEVLKD |
| | amino acid | ELHLDEQEAKFTSQFQYTVLNEITDSMSLGEPSAHYLPSHQLNRNADGIQLGP |
| | sequence; | FDLSQPYIAPIVKSRELFSSNDRNCIHSEFDLSGSNIKYSTGDHLAVWPSNPLE |
| | 14HC_CPR_6 | KVEQFLSIFNLDPETIFDLKPLDPTVKVPFPTPTTIGAAIKHYLEITGPVSRQLF |
| | | SSLIQFAPNADVKEKLTLLSKDKDQFAVEITSKYFNIADALKYLSDGAKWDTV |
| | | PMQFLVESVPQMTPRYYSISSSSLSEKQTVHVTSIVENFPNPELPDAPPVVGVT |
| | | TNLLRNIQLAQNNVNIAETNLPVHYDLNGPRKLFANYKLPVHVRRSNFRLPS |
| | | NPSTPVIMIGPGTGVAPFRGFIRERVAFLESQKKGGNNVSLGKHILFYGSRNTD |
| | | DFLYQDEWPEYAKKLDGSFEMVVAHSRLPNTKKVYVQDKLKDYEDQVFEMI |
| | | NNGAFIYVCGDAKGMAKGVSTALVGILSRGKSITTDEATELIKMLKTSGRYQE |
| | | DVWRRAMAEQSDEAVKYYTLEEIQKHNHSKSTWLILHHKVYDLTKFLEEHP |
| | | GGEEVLREQAGGDATENFEDVGHSTDAREMSKTFIIGELHPDDRPKLNKPPE |
| | | TLITTID* |
| 255 | Saccharomyces | atgccgtttggaatagacaacaccgacttcactgtcctggcggggctagtgcttgccgtgctactgtacgt |
| | cerevisiae and | aaagagaaactccatcaaggaactgctgatgtccgatgacggagatatcacagctgtcagctcgggcaa |
| | Homo sapiens | cagagacattgctcaggtggtgaccgaaaacaacaagaactacttggtgttgtatgcgtcgcagactgg |
| | species of origin; | gactgccgaggattacgccaaaaagttttccaaggagctggtggccaagttcaacctaaacgtgatgtgc |
| | nucleic acid | gcagatgttgagaactacgactttgagtcgctaaacgatgtgcccgtcatagtctcgatttttatctctaca |
| | sequence; | tatggtgaaggagacttccccgacggggcggtcaacCttgaagactttatttgtaatgcggaagcgggtg |
| | 14HC_CPR_6 | cactatcgaacctgaggtataatatgtttggtctgggaaattctacttatAaattctttaatggtgccgcca |
| | | agaaggccgagaagcatctctccgccgcgggcgctatcagactaggcaagctcggtgaagctgatgat |
| | | ggtgcaggaactacagacgaagattacatggcctggaaggactccatcctggaggttttgaaagacgaa |
| | | ctgcatttggacgaacaggaagccaagttcacctctcaattccagtacactgtgttgaacgaaatcactg |
| | | actccatgtcgcttggtgaaccctctgctcactatttgccctcgcatcagttgaaccgcaacgcagacggc |
| | | atccaattgggtcccttcgatttgtctcaaccgtatattgcacccatcgtgaaatctcgcgaactgttctctt |
| | | ccaatgaccgtaattgcatccactctgaatttgacttgtccggctctaacatcaagtactccactggtgacc |
| | | atcttgctgtttggccttccaacccattggaaaaggtcgaacagttcttatccatattcaacctggaccctg |
| | | aaaccatttttgacttgaagcccctggatcccaccgtcaaagtgcccttcccaacgccaactactattggc |
| | | gctgctattaaacactatttggaaattacaggacctgtctccagacaattgttttcatctttgattcagttcg |
| | | cccccaacgctgacgtcaaggaaaaattgactctgctttcgaaagacaaggaccaattcgccgtcgaga |
| | | taacctccaaatatttcaacatcgcagatgctctgaaatatttgtctgatggcgccaaatgggacaccgta |
| | | cccatgcaattcttggtcgaatcagttccccaaatgactcctcgttactactctatctcttcctcttctctgtct |
| | | gaaaagcaaaccgtccatgtcacctccattgtggaaaactttcctaacccagaattgcctgatgctcctcc |
| | | agttgttggtgttacgactaacttgttaagaaacattcaattggctcaaaacaatgttaacattgccgaaa |
| | | ctaacctacctgttcactacgatttaaatggcccacgtaaacttttcgccaattacaaattgcccgtccacg |
| | | ttcgtcgttctaacttcagattgccttccaacccttccaccccagttatcatgatcggtccaggtaccggtgt |
| | | tgccccattccgtgggtttatcagagagcgtgtcgcgttcctcgaatcacaaaagaagggcggtaacaac |
| | | gtttcgctaggtaagcatatactgttttatggatcccgtaacactgatgatttcttgtaccaggacgaatgg |
| | | ccagaatacgccaaaaaattggatggttcgttcgaaatggtcgtggcccattccaggttgccaaacacca |
| | | aaaaagtttatgttcaagataaattaaaggattacgaagaccaagtatttgaaatgattaacaacggtgc |
| | | atttatctacgtctgtggtgatgcaaagggtatggccaagggtgtgtcaaccgcattggttggcatcttatc |
| | | ccgtggtaaatccattaccactgatgaagcaacagagctaatcaagatgctcaagacttcaggtagatac |
| | | caagaagatgtctggAGAAGAGCTatggccgaacaatctgatgaagctgttaagtactacaccttgg |
| | | aagaaatccaaaagcacaaccattctaagtctacctggttgatcttgcatcacaaggtttacgatttgacc |
| | | aagttcttagaagaacatccaggtggtgaagaagtcttgagagaacaagctggtggtgatgctactgaa |
| | | aattttgaagatgttggtcattccaccgatgccagagaaatgtctaagactttcattatcggtgaattgcac |
| | | ccagatgatagaccaaaattgaacaaaccaccagaaaccttgattaccaccatcgattaa |
| 256 | Homo sapiens | MINMGDSHVDTSSTVSEAVAEEVSLFSMTDMILFSLIVGLLTYWFLFRKKKEE |
| | species of origin; | VPEFTKIQTLTSSVRESSFVEKMKKTGRNIIVFYGSQTGTAEEFANRLSKDAHR |
| | amino acid | YGMRGMSADPEEYDLADLSSLPEIDNALVVFCMATYGEGDPTDNAQDFYDW |
| | sequence; | LQETDVDLSGVKFAVFGLGNKTYEHFNAMGKYVDKRLEQLGAQRIFELGLGD |
| | 14HC_CPR_3 | DDGNLEEDFITWREQFWPAVCEHFGVEATGEESSIRQYELVVHTDIDAAKVY |
| | | MGEMGRLKSYENQKPPFDAKNPFLAAVTTNRKLNQGTERHLMHLELDISDS |
| | | KIRYESGDHVAVYPANDSALVNQLGKILGADLDVVMSLNNLDEESNKKHPFP |
| | | CPTSYRTALTYYLDITNPPRTNVLYELAQYASEPSEQELLRKMASSSGEGKELY |
| | | LSWVVEARRHILAILQDCPSLRPPIDHLCELLPRLQARYYSIASSSKVHPNSVHI |
| | | CAVVVEYETKAGRINKGVATNWLRAKEPAGENGGRALVPMFVRKSQFRLPF |
| | | KATTPVIMVGPGTGVAPFIGFIQERAWLRQQGKEVGETLLYYGCRRSDEDYLY |
| | | REELAQFHRDGALTQLNVAFSREQSHKVYVQHLLKQDREHLWKLIEGGAHIY |
| | | VCGDARNMARDVQNTFYDIVAELGAMEHAQAVDYIKKLMTKGRYSLDVWS |
| | | RRAMAEQSDEAVKYYTLEEIQKHNHSKSTWLILHHKVYDLTKFLEEHPGGEE |
| | | VLREQAGGDATENFEDVGHSTDAREMSKTFIIGELHPDDRPKLNKPPETLITT |
| | | ID* |
| 257 | Homo sapiens | ATGATCAACATGGGTGATTCTCACGTTGATACCTCTTCTACTGTTTCTGAAG |
| | species of origin; | CTGTTGCTGAAGAAGTCTCTTTGTTCTCTATGACCGATATGATCCTGTTCTC |
| | nucleic acid | TTTGATCGTTGGTTTGTTGACTTACTGGTTCCTGTTCAGAAAGAAGAAAGAG |
| | sequence; | GAAGTTCCAGAGTTCACCAAGATTCAAACTTTGACCTCTTCCGTCAGAGAAT |
| | 14HC_CPR_3 | CTTCATTCGTTGAGAAGATGAAGAAAACCGGTAGAAACATCATCGTGTTCTA |
| | | CGGTTCTCAAACTGGTACAGCTGAAGAATTTGCTAACAGATTGTCTAAGGAT |
| | | GCCCATAGATATGGTATGAGAGGTATGTCTGCTGATCCAGAAGAATACGATT |
| | | TGGCTGATTTGTCATCCTTGCCAGAAATTGATAATGCCTTGGTTGTTTTCTG |
| | | TATGGCTACTTATGGTGAAGGTGATCCAACTGATAATGCTCAAGATTTCTAC |
| | | GACTGGTTGCAAGAAACTGATGTTGATTTGTCTGGTGTTAAGTTCGCTGTTT |
| | | TCGGTTTGGGTAACAAGACTTACGAACATTTTAACGCTATGGGCAAGTACGT |
| | | TGACAAGAGATTGGAACAATTGGGTGCCCAAAGAATCTTCGAATTAGGTTT |
| | | GGGTGATGATGACGGTAATTTGGAAGAAGATTTCATCACTTGGAGAGAACA |
| | | ATTTTGGCCAGCTGTTTGTGAACATTTTGGTGTTGAAGCTACTGGTGAGGAA |
| | | TCCTCTATTAGACAATACGAATTGGTTGTTCACACCGATATTGATGCTGCTA |
| | | AAGTCTATATGGGTGAGATGGGTAGATTGAAGTCTTACGAAAATCAAAAGC |
| | | CACCATTCGATGCTAAGAATCCATTCTTGGCTGCTGTTACTACCAACAGAAA |
| | | ATTGAATCAGGGTACTGAAAGACACTTGATGCACTTGGAATTGGATATCTCC |
| | | GATTCTAAGATCAGGTACGAATCTGGTGATCATGTTGCTGTTTATCCAGCTA |
| | | ATGATTCCGCCTTGGTTAATCAATTGGGTAAGATTTTGGGTGCCGATTTGGA |
| | | TGTTGTTATGTCCTTGAACAACTTGGACGAAGAGTCCAACAAGAAACATCCA |
| | | TTTCCATGTCCAACCTCTTACAGAACTGCTTTGACTTATTACCTGGACATTA |
| | | CCAATCCACCAAGAACTAATGTCTTGTACGAATTAGCTCAATACGCCTCTGA |
| | | ACCATCCGAACAAGAATTATTGAGAAAGATGGCTTCCTCATCCGGTGAAGGT |
| | | AAAGAATTATACTTGTCCTGGGTTGTTGAAGCCAGAAGGCATATTTTGGCTA |
| | | TATTGCAAGATTGCCCATCCTTAAGACCACCAATTGATCATTTGTGCGAACT |
| | | GTTGCCAAGATTGCAAGCTAGATATTACTCCATTGCCTCCTCATCTAAGGTT |
| | | CATCCAAACTCTGTTCATATTTGCGCCGTTGTTGTTGAATACGAAACAAAAG |
| | | CTGGTAGGATTAACAAGGGTGTTGCTACTAATTGGTTGAGAGCTAAAGAAC |
| | | CTGCTGGTGAAAATGGTGGTAGAGCTTTGGTTCCAATGTTCGTTAGAAAGTC |
| | | CCAATTCAGATTGCCATTCAAAGCTACTACCCCAGTTATTATGGTTGGTCCA |
| | | GGTACTGGTGTTGCTCCTTTTATTGGTTTCATTCAAGAAAGAGCCTGGTTGA |
| | | GACAACAGGGTAAAGAAGTTGGAGAAACCTTGTTGTATTACGGTTGTAGAA |
| | | GATCCGACGAGGATTACTTGTATAGAGAAGAATTGGCTCAGTTCCATAGAG |
| | | ATGGTGCTTTGACACAATTGAACGTTGCCTTTTCTAGAGAACAGTCCCATAA |
| | | GGTTTACGTCCAACACTTGTTGAAACAAGACAGAGAACATCTGTGGAAGTTG |
| | | ATTGAAGGTGGTGCTCATATCTACGTTTGTGGTGATGCTAGAAATATGGCTA |
| | | GAGATGTTCAAAACACCTTCTACGATATTGTCGCTGAATTAGGTGCTATGGA |
| | | ACATGCTCAAGCTGTTGACTACATCAAGAAACTAATGACCAAGGGCAGATAC |
| | | TCATTGGATGTTTGGTCTagaagagctatggccgaacaatctgatgaagctgttaagtactac |
| | | accttggaagaaatccaaaagcacaaccattctaagtctacctggttgatcttgcatcacaaggtttacga |
| | | tttgaccaagttcttagaagaacatccaggtggtgaagaagtcttgagagaacaagctggtggtgatgct |
| | | actgaaaattttgaagatgttggtcattccaccgatgccagagaaatgtctaagactttcattatcggtga |
| | | attgcacccagatgatagaccaaaattgaacaaaccaccagaaaccttgattaccaccatcgattaa |
| 258 | Arabidopsis | MTSALYASDLFKQLKSIMGTDSLSDDVVLVIATTSLALVAGFVVLLWKKTTAD |
| | thaliana species | RSGELKPLMIPKSLMAKDEDDDLDLGSGKTRVSIFFGTQTGTAEGFAKALSEE |
| | of origin; amino | IKARYEKAAVKVIDLDDYAADDDQYEEKLKKETLAFFCVATYGDGEPTDNAA |
| | acid sequence; | RFYKWFTEENERDIKLQQLAYGVFALGNRQYEHFNKIGIVLDEELCKKGAKR |
| | 14HC_CPR_4 | LIEVGLGDDDQSIEDDFNAWKESLWSELDKLLKDEDDKSVATPYTAVIPEYR |
| | | VVTHDPRFTTQKSMESNVANGNTTIDIHHPCRVDVAVQKELHTHESDRSCIH |
| | | LEFDISRTGITYETGDHVGVYAENHVEIVEEAGKLLGHSLDLVFSIHADKEDGS |
| | | PLESAVPPPFPGPCTLGTGLARYADLLNPPRKSALVALAAYATEPSEAEKLKH |
| | | LTSPDGKDEYSQWIVASQRSLLEVMAAFPSAKPPLGVFFAAIAPRLQPRYYSIS |
| | | SSPRLAPSRVHVTSALVYGPTPTGRIHKGVCSTWMKNAVPAEKSHECSGAPIF |
| | | IRASNFKLPSNPSTPIVMVGPGTGLAPFRGFLQERMALKEDGEELGSSLLFFGC |
| | | RNRQMDFIYEDELNNFVDQGVISELIMAFSREGAQKEYVQHKMMEKAAQVW |
| | | DLIKEEGYLYVCGDAKGMARDVHRTLHTIVQEQEGVSSSEAEAIVKKLQTEGR |
| | | YLRDVW* |
| 259 | Arabidopsis | ATGACTTCTGCTTTGTATGCTTCCGATTTGTTTAAGCAGCTCAAGTCAATTA |
| | thaliana species | TGGGGACAGATTCGTTATCCGACGATGTTGTACTTGTGATTGCAACGACGTC |
| | of origin; nucleic | TTTGGCACTAGTAGCTGGATTTGTGGTGTTGTTATGGAAGAAAACGACGGC |
| | acid sequence; | GGATCGGAGCGGGGAGCTGAAGCCTTTGATGATCCCTAAGTCTCTTATGGCT |
| | 14HC_CPR_4 | AAGGACGAGGATGATGATTTGGATTTGGGATCCGGGAAGACTAGAGTCTCT |
| | | ATCTTCTTCGGTACGCAGACTGGAACAGCTGAGGGATTTGCTAAGGCATTAT |
| | | CCGAAGAAATCAAAGCGAGATATGAAAAAGCAGCAGTCAAAGTCATTGACT |
| | | TGGATGACTATGCTGCCGATGATGACCAGTATGAAGAGAAATTGAAGAAGG |
| | | AAACTTTGGCATTTTTCTGTGTTGCTACTTATGGAGATGGAGAGCCTACTGA |
| | | CAATGCTGCCAGATTTTACAAATGGTTTACGGAGGAAAATGAACGGGATAT |
| | | AAAGCTTCAACAACTAGCATATGGTGTGTTTGCTCTTGGTAATCGCCAATAT |
| | | GAACATTTTAATAAGATCGGGATAGTTCTTGATGAAGAGTTATGTAAGAAA |
| | | GGTGCAAAGCGTCTTATTGAAGTCGGTCTAGGAGATGATGATCAGAGCATT |
| | | GAGGATGATTTTAATGCCTGGAAAGAATCACTATGGTCTGAGCTAGACAAG |
| | | CTCCTCAAAGACGAGGATGATAAAAGTGTGGCAACTCCTTATACAGCTGTTA |
| | | TTCCTGAATACCGGGTGGTGACTCATGATCCTCGGTTTACAACTCAAAAATC |
| | | AATGGAATCAAATGTGGCCAATGGAAATACTACTATTGACATTCATCATCCC |
| | | TGCAGAGTTGATGTTGCTGTGCAGAAGGAGCTTCACACACATGAATCTGATC |
| | | GGTCTTGCATTCATCTCGAGTTCGACATATCCAGGACGGGTATTACATATGA |
| | | AACAGGTGACCATGTAGGTGTATATGCTGAAAATCATGTTGAAATAGTTGA |
| | | AGAAGCTGGAAAATTGCTTGGCCACTCTTTAGATTTAGTATTTTCCATACAT |
| | | GCTGACAAGGAAGATGGCTCCCCATTGGAAAGCGCAGTGCCGCCTCCTTTCC |
| | | CTGGTCCATGCACACTTGGGACTGGTTTGGCAAGATACGCAGACCTTTTGAA |
| | | CCCTCCTCGAAAGTCTGCGTTAGTTGCCTTGGCGGCCTATGCCACTGAACCA |
| | | AGTGAAGCCGAGAAACTTAAGCACCTGACATCACCTGATGGAAAGGATGAG |
| | | TACTCACAATGGATTGTTGCAAGTCAGAGAAGTCTTTTAGAGGTGATGGCTG |
| | | CTTTTCCATCTGCAAAACCCCCACTAGGTGTATTTTTTGCTGCAATAGCTCC |
| | | TCGTCTACAACCTCGTTACTACTCCATCTCATCCTCGCCAAGATTGGCGCCAA |
| | | GTAGAGTTCATGTTACATCCGCACTAGTATATGGTCCAACTCCTACTGGTAG |
| | | AATCCACAAGGGTGTGTGTTCTACGTGGATGAAGAATGCAGTTCCTGCGGAG |
| | | AAAAGTCATGAATGTAGTGGAGCCCCAATCTTTATTCGAGCATCTAATTTCA |
| | | AGTTACCATCCAACCCTTCAACTCCAATCGTTATGGTGGGACCTGGGACTGG |
| | | GCTGGCACCTTTTAGAGGTTTTCTGCAGGAAAGGATGGCACTAAAAGAAGA |
| | | TGGAGAAGAACTAGGTTCATCTTTGCTCTTCTTTGGGTGTAGAAATCGACAG |
| | | ATGGACTTTATATACGAGGATGAGCTCAATAATTTTGTTGATCAAGGCGTA |
| | | ATATCTGAGCTCATCATGGCATTCTCCCGTGAAGGAGCTCAGAAGGAGTATG |
| | | TTCAACATAAGATGATGGAGAAGGCAGCACAAGTTTGGGATCTAATAAAGG |
| | | AAGAAGGATATCTCTATGTATGCGGTGATGCTAAGGGCATGGCGAGGGACG |
| | | TCCACCGAACTCTACACACCATTGTTCAGGAGCAGGAAGGTGTGAGTTCGTC |
| | | AGAGGCAGAGGCTATAGTTAAGAAACTTCAAACCGAAGGAAGATACCTCAG |
| | | AGATGTCTGGTGA |
| 260 | Alosa | MADVQSEAPTQPDLMEADEPLLSNLDIFLFSLIAGLVIYWFTFRKKPEPIPEFK |
| | sapidissima | KLDMPAPSTRETSFIEKMKKTGRNIVVFYGSQTGTAEEFSNRLSKDAQRYGM |
| | species of origin; | KGMAADPEEYDLSELARLSEIENSLAIFCMATYGEGDPTDNAQDFYDWLQEG |
| | amino acid | DADLDGLNFAVFALGNKTYEHYNAMGKYVDKRLEELGGKRVFDLGMGDDD |
| | sequence; | GNLEEDFVSWREQFWPAMCEHFGVEATGEESSIRQYELKVHDDLNMNKVYT |
| | 14HC_CPR_24 | GELGRLKSFESQKPPFDSKNPFLAPVTVNRKLNKGGDRHLMHLELDITGSKIR |
| | | YESGDHVAVYPINDTTIVTKLGDILGVDLDTVISLNNLDEESNKKHPFPCPTTY |
| | | RTALTHYLDITHPPRTNVLYELAQYATDPKDQENMRKMASSSSEGKALYQSF |
| | | VLDPSRNILAILEDMPSLKPPADHLCELLPRLQARYYSIASSSKVHPNSIHVCC |
| | | VVVEYKTSTGRTNKGVATNWLKSKLPTDNGHKATVPMYVRKSQFRLPFKAT |
| | | NPVIMIGPGTGIAPFIGFVQERGWLKEQGKEVGETVLYYGCRHKNEDYLYQEE |
| | | LEEFEKTGVLTKLNVAFSRDQDQKVYVQHLIKNNKEEIWNQVHSQSAHIYICG |
| | | DARNMARDVQAAFYDIAEELGGMTHAQATDYVKKLMTKGRYSQDVWS |
| 261 | Alosa | ATGGCGGATGTACAAAGCGAGGCTCCCACTCAACCCGACCTAATGGAAGCGG |
| | sapidissima | ATGAACCTCTGTTATCAAACTTGGATATTTTTCTTTTTAGCCTGATTGCTGG |
| | species of origin; | GTTAGTTATCTATTGGTTCACGTTTAGAAAGAAGCCGGAACCAATTCCCGAG |
| | nucleic acid | TTCAAGAAGCTAGACATGCCTGCTCCCAGTACACGTGAAACATCCTTCATAG |
| | sequence; | AAAAGATGAAGAAGACAGGGAGAAATATTGTCGTTTTCTATGGCTCACAAA |
| | 14HC_CPR_24 | CAGGAACTGCAGAGGAGTTTTCCAATAGACTTTCCAAGGATGCACAACGTTA |
| | | CGGTATGAAGGGTATGGCTGCAGACCCAGAAGAGTATGACCTTTCAGAATTG |
| | | GCGAGGTTGAGCGAAATAGAAAATTCATTAGCCATTTTTTGTATGGCCACAT |
| | | ACGGCGAAGGTGATCCAACGGACAACGCTCAGGATTTTTACGATTGGTTGCA |
| | | AGAGGGGGACGCTGATCTAGACGGTCTTAACTTTGCTGTTTTCGCACTAGGT |
| | | AACAAGACGTACGAACATTACAATGCAATGGGTAAGTATGTCGATAAGCGT |
| | | TTAGAGGAACTAGGCGGCAAAAGAGTCTTCGACCTTGGTATGGGGGACGACG |
| | | ATGGGAATCTAGAAGAGGACTTCGTAAGTTGGAGAGAACAGTTCTGGCCTG |
| | | CCATGTGCGAGCATTTTGGCGTAGAGGCAACAGGCGAAGAGTCTTCTATACG |
| | | TCAGTACGAACTTAAAGTACACGACGACTTAAATATGAATAAGGTATACAC |
| | | AGGAGAGTTGGGTAGGTTAAAGTCCTTTGAGTCACAAAAGCCGCCATTCGAC |
| | | TCCAAAAATCCCTTTTTAGCTCCAGTAACAGTAAATCGTAAGCTGAACAAGG |
| | | GCGGTGATCGTCATTTAATGCATCTGGAATTAGACATTACAGGGAGCAAGAT |
| | | TAGATATGAGTCAGGCGATCATGTGGCGGTTTATCCTATCAATGACACTACT |
| | | ATCGTAACGAAACTGGGTGATATTTTAGGGGTAGACCTGGACACTGTTATA |
| | | AGTTTAAATAATCTTGATGAGGAATCCAATAAGAAGCATCCGTTCCCGTGTC |
| | | CAACAACCTACAGAACAGCTTTAACACATTATTTAGACATAACACATCCACC |
| | | CAGGACTAACGTTCTATATGAACTAGCTCAGTACGCGACGGACCCCAAGGAT |
| | | CAGGAGAACATGAGAAAGATGGCAAGCTCATCTTCCGAGGGAAAAGCATTG |
| | | TATCAGTCATTTGTGCTAGACCCCTCAAGGAACATACTAGCCATATTAGAAG |
| | | ACATGCCATCACTAAAACCGCCCGCCGACCATTTGTGTGAATTGCTACCGAG |
| | | GCTGCAAGCACGTTATTATAGCATCGCTTCAAGCTCCAAGGTACATCCCAAC |
| | | AGTATTCACGTGTGCTGTGTCGTCGTAGAGTACAAGACCTCCACGGGCAGAA |
| | | CCAACAAGGGGGTAGCTACAAACTGGCTTAAGTCTAAACTACCGACGGATAA |
| | | TGGCCACAAAGCAACGGTGCCCATGTACGTTCGTAAGAGTCAATTCAGGTTG |
| | | CCTTTCAAAGCTACTAACCCTGTCATAATGATTGGACCAGGGACGGGAATTG |
| | | CACCGTTCATTGGGTTCGTCCAGGAAAGAGGCTGGTTGAAGGAACAGGGCAA |
| | | AGAAGTTGGAGAAACAGTTCTGTATTATGGATGTAGGCATAAAAATGAGGA |
| | | CTACCTATACCAAGAAGAGTTGGAGGAATTCGAAAAGACGGGCGTCCTAACA |
| | | AAGTTGAACGTGGCCTTCTCTAGGGATCAGGACCAGAAGGTATATGTACAAC |
| | | ACTTAATAAAGAACAATAAAGAGGAAATATGGAACCAAGTGCACTCTCAGT |
| | | CAGCACACATCTATATCTGCGGGGACGCGAGGAACATGGCGAGAGATGTGCA |
| | | AGCTGCGTTCTATGACATCGCTGAAGAATTGGGGGGGATGACGCACGCTCAA |
| | | GCCACGGATTATGTAAAGAAGCTGATGACCAAAGGCAGGTACAGTCAGGAC |
| | | GTGTGGTCA |
| 262 | Circinella minor | MAYSKKTTSSLLAITPLDIFLGGILGLGTIIWISRQFRSSSFKYDNSNSKPDNQK |
| | species of origin; | TAVNTGPERNFVKLMKQQNRQVIFFYGSQTGTAEDYASRLAKECSQKYGVSA |
| | amino acid | MTADIELYDMTYLDAVPSDNLVVFVMATYGEGEPTDNAVEFWELFNGEEPE |
| | sequence; | FSESTTLENLKYIVFGLGNKTYEHYNAVARVLDTRLSELGAKRIGERGEGDDD |
| | 14HC_CPR_28 | ASLEEDFLAWQENMWPHFCNALGVDESNAQSGPRQVMFGVEELDAVDKTS |
| | | LFLGELGEWKNPEQLHQTVVYDAKHPYQAPISLSRDLFNESDDRHCLHLEID |
| | | LSKSNLTYQTGDHIAIWPTNNEVEVERFGSILGLSDKLDTAIMVEALDSAASK |
| | | KHPFPVPTTYRALFRHYLDICAPASRQTLMSLVEYAPTESSKETLRQLATDKD |
| | | VYRIKVGEAMRNLAEVLELCQDDDEGTATKMSSGFFSTVPFDLIVESLSRLQP |
| | | RYYSISSSASTSPKSVAVTAVTLAYQPTKEEDRTVYGVNTNYLWRIHAPEDNS |
| | | IPAYDLNGPRDTLKNQKIPVHIRRSQFKLPRNTKLPVIMVGPGTGVAPFRGFV |
| | | QERVYQKQQGKEVGPTVLFYGCRRSDQDFLYKDEWPQLFKDLGEESRIITAFS |
| | | RETEQKVYVQHRLKEHGEKMWEYMEQGALLYVCGDAKNMARDVNQTFVSF |
| | | AIEFGGKTEQQAQDYVKNLRTSGRYQEDVWS |
| 263 | Circinella minor | ATGGCGTATAGTAAGAAGACGACAAGTAGCCTGCTTGCTATAACACCCCTTG |
| | species of origin; | ATATCTTTCTGGGCGGAATCTTAGGGCTGGGCACTATTATATGGATTAGCAG |
| | nucleic acid | GCAATTTAGATCAAGCTCTTTTAAATATGACAACAGTAATAGTAAACCCGAT |
| | sequence; | AATCAAAAAACGGCCGTCAATACGGGGCCAGAAAGGAATTTCGTCAAATTG |
| | 14HC_CPR_28 | ATGAAACAACAGAACAGACAGGTAATTTTTTTCTATGGCAGCCAGACCGGA |
| | | ACTGCGGAGGATTACGCGAGCAGGTTGGCGAAGGAATGCAGCCAAAAATAT |
| | | GGCGTGTCTGCAATGACTGCCGATATAGAACTTTACGATATGACCTACTTGG |
| | | ACGCCGTACCGTCAGATAACCTAGTGGTTTTCGTTATGGCTACCTACGGAGA |
| | | AGGGGAACCGACGGACAATGCGGTCGAGTTTTGGGAGCTTTTTAACGGGGA |
| | | AGAGCCGGAGTTTTCAGAGAGCACAACGCTAGAAAATCTGAAATACATCGT |
| | | GTTCGGATTAGGGAACAAAACGTACGAGCATTACAACGCCGTAGCCAGGGTG |
| | | CTGGACACGAGACTAAGCGAGCTAGGGGCCAAGAGAATCGGTGAGCGTGGCG |
| | | AGGGTGACGATGACGCAAGTCTAGAGGAAGATTTTTTAGCGTGGCAGGAAA |
| | | ATATGTGGCCGCATTTCTGTAATGCGCTGGGAGTAGACGAAAGTAATGCGCA |
| | | GTCCGGGCCTCGTCAAGTTATGTTCGGCGTAGAAGAACTTGACGCCGTAGAC |
| | | AAGACGAGTCTTTTTTTAGGTGAACTAGGAGAGTGGAAAAACCCTGAGCAG |
| | | TTACACCAGACAGTAGTGTATGATGCGAAGCATCCTTATCAGGCGCCTATTA |
| | | GCTTATCTAGGGACCTTTTTAACGAGAGCGACGATCGTCACTGCCTGCACTT |
| | | AGAGATTGACTTGAGTAAATCCAACCTAACCTACCAAACCGGGGATCACATC |
| | | GCGATATGGCCTACGAATAACGAGGTTGAGGTGGAAAGATTCGGAAGTATA |
| | | CTGGGTCTAAGTGACAAGCTGGATACTGCCATCATGGTTGAGGCGCTGGACA |
| | | GTGCAGCTAGCAAGAAACATCCATTCCCCGTACCTACAACTTATAGGGCTCT |
| | | GTTTAGACATTATCTGGACATTTGCGCACCCGCAAGCCGTCAGACATTGATG |
| | | AGCCTAGTAGAGTATGCACCCACGGAGTCTAGCAAGGAGACACTGAGACAGC |
| | | TTGCCACGGATAAGGATGTGTATAGGATAAAGGTAGGGGAGGCTATGAGAA |
| | | ACTTGGCCGAGGTTCTGGAACTTTGCCAGGATGATGACGAAGGAACGGCTAC |
| | | AAAGATGTCCTCAGGTTTCTTCTCCACCGTACCATTTGATCTTATTGTCGAG |
| | | AGTCTAAGCAGGCTTCAACCCAGGTACTACTCTATCTCCAGTAGCGCGTCCA |
| | | CCTCACCGAAATCTGTGGCTGTAACTGCGGTAACGTTGGCCTACCAACCTAC |
| | | CAAAGAAGAGGATAGGACGGTATATGGTGTAAACACAAATTATTTATGGAG |
| | | AATCCACGCCCCCGAGGACAACTCCATACCAGCCTACGATTTAAATGGTCCA |
| | | AGGGACACGCTAAAGAACCAGAAGATCCCCGTGCACATCAGACGTTCACAGT |
| | | TCAAGCTGCCGCGTAACACGAAGTTGCCGGTGATAATGGTCGGTCCTGGGAC |
| | | CGGCGTTGCCCCTTTCCGTGGCTTCGTTCAGGAGCGTGTGTATCAAAAGCAG |
| | | CAGGGTAAAGAGGTTGGGCCGACCGTTCTTTTTTACGGATGCAGGCGTAGTG |
| | | ATCAAGATTTTTTGTACAAAGACGAATGGCCACAACTGTTCAAAGACCTTGG |
| | | CGAAGAGAGCAGGATTATAACCGCCTTTTCAAGAGAGACTGAGCAAAAAGT |
| | | TTATGTCCAACACAGATTGAAGGAGCATGGGGAGAAAATGTGGGAATACAT |
| | | GGAGCAAGGCGCATTGCTATACGTATGCGGGGACGCCAAGAATATGGCCCGT |
| | | GATGTTAACCAGACGTTCGTTAGCTTCGCCATTGAATTTGGTGGTAAAACGG |
| | | AACAGCAGGCCCAGGATTATGTGAAGAATCTGCGTACAAGTGGAAGGTACC |
| | | AAGAGGACGTCTGGTCC |
| 264 | Rhizopus | MTRNNSHHLLDTVDLILLGTIGLGTVAWFARHQIANRLFKSDSTNKSEVKDE |
| | delemar species | AKTPKQERNFVKVMQQQGRRVIFFYGSQTGTAEDFASRLAKECTQKYGVSA |
| | of origin; amino | MTADIEQYDLSYLDSVPEDSLVFFIMATYGEGEPTDNAVDFWDLLAEEVPEF |
| | acid sequence; | SNDDGEGKPLQKLRYVAFGLGNKTYEHYNEVIRKVDNRLLSLGAKRIGERGE |
| | 14HC_CPR_29 | GDDDGTLEEDFLAWQEEMWPAFCEALGVDESNAHSGPRQAIFKIEELTAYD |
| | | QAKVYLGEIGEWLKEGASIVYDAKRPYNAPITSKDIFKAGDRHCLHLEIDISNT |
| | | NLSYQTGDHVAIWPTNNEVEVNRLAKLLGLQNKLDTVIHVQSLDPAASKKYP |
| | | FPVPTTYRAVFRHYLDICSAVPRQVLMSLIEYAPTEASKEALRKLATDKDEYR |
| | | VHVGDVTRNLGEVLQMLAESESLELDGAFSSVPFDLVIESISRLQPRYYSISSSS |
| | | KENPKKIAVTAVTLQYTPEHGSPRTVYGVNTNYLWRVHEAVNNLTPNSVIPE |
| | | YNLTGPRDSLFSQEDKVARIPVHVRRSQFKLPRNPTVPIIMIGPGTGVAPFRGF |
| | | VRERALQKKENKPVGPTILFFGCRNRAEDFLYEEEWPELFEVLGGDSRIITAF |
| | | SRETEKKVYVQHRLMENGQEMWNLLEKGAYVYVCGDAKNMARDVNQTFV |
| | | RFAQQFGGMDENRSQDYVKNLRNTGRYQEDVWS |
| 265 | Rhizopus | ATGACAAGAAACAATTCACACCATTTGCTTGACACTGTAGATCTGATTTTGT |
| | delemar species | TGGGCACAATAGGCCTTGGTACAGTCGCCTGGTTTGCTAGACACCAGATTGC |
| | of origin; nucleic | TAATAGGCTTTTCAAGAGCGATTCCACGAATAAGTCCGAAGTTAAAGATGA |
| | acid sequence; | AGCGAAGACACCTAAGCAAGAACGTAACTTTGTTAAGGTAATGCAGCAACA |
| | 14HC_CPR_29 | AGGCAGGAGAGTAATCTTCTTTTATGGGTCCCAGACGGGTACAGCGGAGGAC |
| | | TTCGCTAGTCGTCTAGCTAAAGAGTGTACCCAAAAGTATGGTGTTTCTGCTA |
| | | TGACCGCGGACATTGAGCAATATGACCTTAGCTACCTTGACTCAGTGCCCGA |
| | | GGATAGCCTAGTGTTTTTCATCATGGCTACTTATGGCGAAGGAGAGCCAACG |
| | | GATAACGCTGTCGATTTTTGGGACCTACTTGCGGAAGAGGTACCCGAGTTTA |
| | | GCAATGACGACGGTGAAGGGAAGCCGTTGCAGAAATTAAGGTATGTAGCGT |
| | | TTGGGCTAGGCAATAAAACGTATGAGCATTATAATGAAGTCATCCGTAAAG |
| | | TCGACAATAGACTGCTGTCACTTGGAGCAAAGAGGATTGGTGAGCGTGGGG |
| | | AGGGGGATGATGACGGCACGTTAGAAGAGGACTTTCTTGCCTGGCAAGAGG |
| | | AAATGTGGCCCGCTTTTTGTGAGGCGTTGGGGGTAGACGAATCAAATGCTCA |
| | | CAGCGGGCCGCGTCAGGCCATCTTCAAGATAGAAGAGCTTACGGCTTATGAT |
| | | CAAGCCAAAGTCTACCTGGGAGAGATAGGGGAGTGGCTGAAGGAGGGAGCC |
| | | TCTATAGTTTATGATGCAAAGAGGCCATACAACGCTCCTATTACCTCCAAAG |
| | | ACATCTTCAAAGCTGGGGATAGACATTGCTTACATTTGGAAATAGATATTA |
| | | GTAATACTAACCTGTCATACCAGACGGGAGACCACGTTGCGATTTGGCCCAC |
| | | CAATAACGAAGTTGAGGTAAATCGTCTTGCAAAACTGCTAGGTCTGCAAAA |
| | | TAAATTAGATACGGTAATTCACGTTCAATCCCTAGATCCGGCAGCTTCAAAA |
| | | AAATACCCGTTCCCCGTACCCACGACCTATAGGGCGGTGTTCAGGCACTATC |
| | | TGGACATATGCTCCGCAGTACCGAGGCAAGTACTTATGAGCTTAATAGAATA |
| | | CGCACCCACGGAAGCCTCCAAGGAAGCGTTGAGGAAGCTTGCGACGGATAAA |
| | | GATGAGTATCGTGTCCATGTAGGAGATGTTACACGTAATTTGGGTGAGGTT |
| | | TTACAGATGCTAGCTGAGAGCGAGTCCCTAGAGCTGGATGGCGCGTTTTCCT |
| | | CAGTACCCTTCGACCTAGTTATTGAATCAATATCAAGATTGCAACCGAGGTA |
| | | CTACAGTATCTCTAGCAGTTCAAAGGAGAATCCTAAGAAGATAGCAGTGACG |
| | | GCAGTGACCTTACAATATACACCGGAGCATGGTTCTCCTAGAACTGTTTACG |
| | | GTGTAAACACTAACTATTTATGGCGTGTTCACGAAGCCGTTAACAATTTAAC |
| | | GCCGAACTCAGTCATTCCTGAATATAATTTGACCGGACCGAGGGACAGTCTA |
| | | TTCTCACAGGAGGACAAAGTTGCCAGGATCCCTGTGCATGTTCGTAGATCCC |
| | | AATTTAAGCTTCCTAGAAACCCCACGGTTCCTATAATTATGATAGGGCCCGG |
| | | AACTGGCGTTGCTCCCTTTAGGGGGTTCGTAAGGGAACGTGCGTTGCAAAAA |
| | | AAGGAAAACAAACCGGTAGGTCCAACTATCTTATTTTTCGGGTGCAGGAACC |
| | | GTGCCGAAGACTTTCTATATGAAGAGGAATGGCCCGAACTATTTGAGGTGCT |
| | | TGGGGGGGACTCTCGTATTATTACCGCATTTAGTCGTGAGACAGAGAAAAA |
| | | AGTCTATGTCCAACACAGGTTGATGGAAAACGGACAAGAAATGTGGAACTT |
| | | ATTGGAGAAAGGCGCGTATGTGTACGTTTGCGGGGACGCCAAGAATATGGC |
| | | AAGAGACGTCAATCAAACATTCGTGAGGTTTGCCCAGCAATTCGGAGGGATG |
| | | GATGAGAACAGGTCCCAGGACTATGTCAAGAATTTACGTAATACAGGGCGTT |
| | | ACCAGGAAGACGTTTGGTCC |
| 266 | Mucor ambiguus | MDPEPATALFDTTDLVLLTAIGLGTAAWFSRHQLAELFGKKKQKVVTAGTN |
| | species of origin; | TAQPKRETNFVKVMEQQGRRVIVFYGSQTGTAEGFAARIAKECSQRFGVSCM |
| | amino acid | TADLELYDLTYLDQVPKDNLVVFVLATYGEGDPTDNAMEFWDLVTEEEPTF |
| | sequence; | SESSEDRPLTNLNYVMFGLGNKTYDQYNEVSRRINKCLLKLGAHLVGEAGEG |
| | 14HC_CPR_30 | DDDASMEEDYLAWKEKMWPAFCETLGVDESSARSGPRQPVYMVEELPEYE |
| | | KNEVYFGELAEKAKNNAKIIYDAKRPYIATMTSRELFHGADRHCMHIDVDISG |
| | | TNLSYNTGDHLAIWPTNNEIEVNRLASVLGLTDKLDTVVMVKAIDPMAPKK |
| | | HPFPVPTTYRSIFRYYIEICAPISRQVLNSFVEYAPSEEAKANLTRLATDKDEYR |
| | | LQVGDACRNLGEVLQLVTGADTRPGLFANVPFDLIVETVARMQPRFYSISSSA |
| | | KESPKAISATVVTLAYQPLPTPHRTVYGVNTNFLYSIQMQNESTVADGQEHPP |
| | | YDLAGPRQAYLGSDGYCHKIPVHVRHSQFKLPRNPSVPVIMVGPGTGVAPFR |
| | | GFVRERVVEKRNGKPVGTTLLFYGSRNQAQDFLYADEWPELFETLGGESRIV |
| | | TAFSRDQAQKIYVQHRLQEHGAQVWELLQKGGYVYVCGDAKSMARDVNQTF |
| | | INCAIEYGGMTDDKAHEYVKGLRTTGRYQEDVWS |
| 267 | Mucor ambiguus | ATGGACCCCGAGCCCGCGACGGCTCTTTTTGACACAACAGACTTAGTATTAT |
| | species of origin; | TAACCGCTATAGGTCTAGGGACAGCAGCGTGGTTCAGCAGACATCAGTTGGC |
| | nucleic acid | GGAGTTGTTCGGCAAAAAGAAGCAAAAGGTTGTCACTGCGGGTACGAACAC |
| | sequence; | TGCCCAGCCGAAGAGAGAGACTAATTTCGTGAAGGTAATGGAACAACAAGG |
| | 14HC_CPR_30 | CCGTAGGGTCATCGTATTTTATGGTAGTCAGACTGGAACTGCGGAGGGTTTC |
| | | GCGGCTAGAATTGCAAAAGAGTGTTCTCAGCGTTTTGGCGTGTCTTGTATGA |
| | | CTGCAGACCTAGAGTTATACGACCTAACGTATCTTGACCAGGTGCCCAAAGA |
| | | TAATCTGGTCGTCTTTGTATTAGCCACGTATGGCGAAGGGGACCCAACGGAC |
| | | AATGCCATGGAGTTCTGGGACCTTGTAACAGAGGAAGAGCCAACCTTTAGTG |
| | | AGTCTTCTGAGGATCGTCCATTAACCAACCTGAACTACGTTATGTTCGGTCT |
| | | AGGTAATAAGACATACGACCAGTATAATGAGGTTTCTAGAAGAATCAATAA |
| | | ATGTCTTCTGAAACTTGGTGCCCATCTTGTGGGTGAGGCTGGAGAAGGCGAC |
| | | GACGATGCTTCCATGGAAGAAGACTATCTAGCTTGGAAGGAGAAGATGTGG |
| | | CCTGCTTTTTGTGAAACGTTGGGGGTGGACGAGAGTTCTGCACGTTCCGGAC |
| | | CTCGTCAACCCGTATATATGGTTGAAGAGTTACCGGAATATGAGAAAAATG |
| | | AGGTTTATTTTGGAGAATTGGCCGAGAAGGCAAAAAATAATGCAAAAATTA |
| | | TTTACGACGCCAAGCGTCCTTATATTGCTACCATGACCAGTCGTGAGCTGTT |
| | | TCACGGTGCTGATCGTCACTGTATGCACATAGACGTGGATATTAGCGGGACC |
| | | AACCTAAGTTATAATACGGGTGATCACCTAGCAATCTGGCCCACCAATAACG |
| | | AGATCGAAGTTAATAGGCTAGCGTCTGTGTTAGGTCTTACCGATAAGTTGGA |
| | | TACTGTCGTTATGGTTAAGGCGATCGACCCCATGGCGCCGAAAAAACATCCT |
| | | TTTCCCGTACCGACGACTTATAGATCAATCTTTAGATACTATATTGAGATAT |
| | | GTGCTCCCATATCCAGGCAAGTCTTGAACAGCTTTGTGGAGTACGCACCTTC |
| | | AGAAGAAGCTAAAGCTAACCTGACTCGTCTGGCCACCGACAAGGATGAATAC |
| | | AGACTGCAGGTCGGAGATGCCTGCAGGAATTTGGGAGAGGTCCTGCAGCTTG |
| | | TTACAGGTGCGGATACCAGGCCCGGATTATTCGCAAACGTTCCGTTTGATCT |
| | | AATAGTCGAGACGGTGGCCAGGATGCAGCCGCGTTTTTACAGTATCAGCAGT |
| | | AGTGCTAAGGAATCTCCCAAGGCGATTAGCGCAACCGTTGTTACGCTGGCCT |
| | | ATCAACCATTGCCTACCCCTCATAGAACCGTGTATGGCGTTAATACTAACTT |
| | | TTTGTATTCCATACAGATGCAGAACGAGTCTACTGTCGCCGACGGACAGGAA |
| | | CACCCGCCCTATGACCTAGCCGGGCCGAGACAGGCATACCTGGGATCCGATG |
| | | GGTACTGTCATAAGATTCCAGTACACGTTAGGCACAGCCAATTTAAGCTGCC |
| | | GAGAAACCCGTCCGTCCCCGTCATCATGGTCGGACCAGGAACCGGGGTAGCG |
| | | CCTTTCAGAGGTTTCGTCCGTGAGAGGGTCGTTGAGAAGAGGAACGGGAAAC |
| | | CAGTAGGAACAACACTACTTTTCTATGGGTCACGTAACCAGGCGCAAGATTT |
| | | CTTATACGCCGACGAGTGGCCCGAACTATTTGAGACGCTGGGCGGAGAATCA |
| | | AGAATTGTAACTGCGTTCTCAAGAGACCAAGCTCAAAAAATATACGTACAGC |
| | | ATAGACTACAGGAGCATGGCGCTCAAGTCTGGGAATTGCTTCAAAAGGGAG |
| | | GCTATGTATATGTCTGTGGAGATGCTAAGTCCATGGCGCGTGATGTCAACCA |
| | | AACTTTCATAAATTGTGCTATCGAGTATGGAGGGATGACCGACGACAAGGCG |
| | | CATGAGTATGTGAAGGGTCTACGTACCACTGGCAGATACCAGGAAGATGTCT |
| | | GGTCA |
| 268 | Thamnostylum | MAKNTTLNKQVVLLISALGLSAVAYMASRYFFGNSASTPEQSKKPIQPEEKED |
| | piriforme | ESNFVQTMKKQNRKVVIFYGSQTGTAEDCAQRLGKQCKKQFGVPALVLDIEQ |
| | species of origin; | CNMSYLDQIPEDCVAIFVMATYGEGEPTDNATDFWEMLEAHPEFSQSDNLK |
| | amino acid | NLRYFIFGLGNSSYTYYNWVSKTVDKKLTELGATRLGKLGLGDDEKSLEDDF |
| | sequence; | EAWQEGMWPIFGEAVETDAASNISGGHEPTYHVVELTEHDGHVYSGELGDK |
| | 14HC_CPR_31 | SQALYSGKKPYPAPITTRDLLNGSDRHCLHLEVDISGSGMSYTTGDHIAIWPT |
| | | NSEDEVLSLARAIGLENKLDTVICVTAADETSAKQSPFPQPTTYRAMLRHYLD |
| | | ICQMPSRQTLEMLVPFAPSPEAAETLEKLAKDKDEHRRVVLGPVRNLAAVLS |
| | | YASSGAAYKIPSDVLLECLGRLQPRYYSISSSALESPNSVSVTAVTLKFNPEPTP |
| | | ERTVFGVNTNYLWAVHTALNDDDEAIQVETEYSIGGPNGQYFDEKLKTARLP |
| | | VHIRQSNFKLPADTTKPVIMIGPGTGVAPFRAFVRERAYQKKHLGKSIGPTILY |
| | | FGCRRSNEDFLYQEEWPELFNELGGSSRLITAFSRETDRKVYVQDKLNENGP |
| | | ETWDLINNKGAYVYVCGDAKRMAKDVQSALVSFAKQYGSYDDAGAADYISK |
| | | LRDAGRYQEDVWA* |
| 269 | Thamnostylum | atggcaaaaaatactacattaaacaagcaagtggtgcttctcatcagtgccctgggcctgagcgctgttg |
| | piriforme | cctacatggccagccgatacttttttggaaacagcgcaagcacccctgaacaatcgaaaaagcccattca |
| | species of origin; | accagaagaaaaagaagacgaatcaaactttgtgcaaaccatgaagaagcagaatcgtaaagtagtga |
| | nucleic acid | tcttttatggttcgcaaacaggcacggctgaagattgtgcgcagcgacttggcaagcagtgcaaaaagc |
| | sequence; | aattcggcgttcctgctttggttcttgatatcgaacaatgcaacatgtcgtacttggatcagattcccgagg |
| | 14HC_CPR_31 | actgtgttgcgatatttgtcatggcaacttatggcgaaggtgaacccactgacaacgccactgacttttgg |
| | | gaaatgcttgaagcccatcctgaattttcccaaagcgacaacctcaagaatctccgatacttcatcttcgg |
| | | acttggcaacagctcctatacctattacaactgggtctccaaaactgtcgacaagaaactcactgaattag |
| | | gcgcgacacgacttggcaaacttggcttgggcgacgacgaaaagtcgctcgaagacgactttgaagcat |
| | | ggcaggaaggcatgtggcctatttttggagaagctgtggaaacggacgccgccagcaatatcagcggc |
| | | ggacacgagcccacataccatgtggtggagctaaccgaacatgacggccacgtgtattctggtgaattg |
| | | ggagacaaatcgcaagctctctatagcggcaagaagccttacccagcacctattaccacgcgcgatttgt |
| | | tgaacggatcggatcgccactgtctacacctggaagtcgatatcagtggatcaggcatgagctataccac |
| | | tggcgatcatattgccatctggcccacaaacagcgaagacgaggtcttgagcttggcacgcgccattggt |
| | | ctcgaaaacaagctggataccgtcatttgcgttactgctgcggatgagacgtccgccaagcaatcaccgt |
| | | tccctcagccaacaacataccgtgccatgctgcgacactacttggatatttgccaaatgccatcacggca |
| | | gacactggagatgctcgtaccttttgcaccgtcgcccgaagctgcagagactctagaaaagctggcaaa |
| | | ggacaaggatgagcatcgtcgagtggttctgggaccagttcgtaacttggctgctgtgctgagttatgcgt |
| | | cgtcgggcgctgcatacaagatcccatccgatgtgctgcttgagtgccttggtcgactacagccccgatat |
| | | tactcgatttcctcctctgccttggagagcccaaacagtgtcagtgtcactgccgttacactcaaattcaac |
| | | cccgaaccaacgcctgagcgcactgtctttggcgtcaataccaactacctgtgggccgtccataccgcgc |
| | | tcaacgatgacgacgaggcaatccaagttgaaaccgagtattccatcggtggacctaacggtcaatactt |
| | | tgacgagaagctcaaaactgcaagattgcctgttcacatccgccaatcaaacttcaagctgccagcagac |
| | | acaactaagccagtgatcatgattggccccggcaccggtgtcgctcctttccgagccttcgttcgcgagcg |
| | | tgcctatcaaaagaagcaccttggcaaatccatcggtccaactattctgtactttggctgccgacgttcca |
| | | acgaggactttttgtatcaggaagagtggccagagctctttaacgaattgggcggctcctctcgattgatc |
| | | actgcattctcccgggaaacggatcgaaaagtatacgtccaagacaagctcaacgagaacggcccaga |
| | | gacttgggatttgattaataacaagggcgcctatgtctatgtttgtggcgacgctaagcgtatggccaaag |
| | | acgtccagtccgcacttgttagttttgcaaagcagtatggcagctatgacgacgctggtgcagctgactac |
| | | atcagcaagttaagagatgccggacgatatcaagaagacgtctgggcataa |
| 270 | Cunninghamella | MAQQTPAVIDTLDLILLGSIGLGTIAWFTRRQLSERLFGTGQSNATPKPTTPQ |
| | elegans species | APKRERNFVKVMEQQGRKVIFFYGSQTGTAEDFASRLAKQCSQKYGVSCMTA |
| | of origin; amino | DIEMYDLSYLDTLSEDSLVCFVMATYGEGEPTDNAVDFWEQFITDESPVFSEG |
| | acid sequence; | GETLENLRYLMFGLGNKTYEHYNAVARILDKKLTGLGAKRIGERGEGDDDGS |
| | 14HC_CPR_32 | LEEDFLAWQESMWPTFCDALGVDENNAQQGPRQASYSVDELKEEYKNDDV |
| | | YFGELGTKSKDSSRVVYDAKRPYNAPITTRELFNSSERHCLHVDIDISGTNLSY |
| | | QTGDHVAMWPTNNEDEVLRLANILGLQDKLDNVISVKAIDPAAPKQHPFPV |
| | | PTTYRAIFRHYIDICAPASRQSLMSFVEFAPTDSAKDLLKLLATDKDEYRLKVG |
| | | EAVRNLGEVLELVSGNDKDTQPGSFTSVPFDLIVETIPRLQPRYYSISSSSKENS |
| | | SIISATCVTLAYQPDPTPDRTVYGVNTNFLYRIHTQSSDNDIQGLPKYDLAGPR |
| | | KAFLNEQGQSHKLPIHIRRSQFKLPRNTSCPVIMIGPGTGVAPFRGFVRERAL |
| | | QKKEGKSVGPTVLFFGNRHSEHDFLYSDEWPELFNTLGDDSKLITAFSRETE |
| | | HKVYVQHRLEENGKDIWQLLEKGAYIYVCGDARNMARDVNQTFVNLAMEY |
| | | GEKTEQKALDYVKSLRNTGRYQEDVWS |
| 271 | Cunninghamella | ATGGCACAGCAAACACCAGCCGTTATCGACACTTTGGATCTTATTTTGTTAG |
| | elegans species | GCTCCATCGGCCTGGGAACAATCGCCTGGTTTACACGTAGACAGTTATCTGA |
| | of origin; nucleic | GAGATTATTTGGGACTGGGCAATCAAATGCAACGCCAAAACCCACGACTCCA |
| | acid sequence; | CAGGCTCCGAAGCGTGAGAGGAACTTTGTTAAGGTTATGGAACAGCAAGGG |
| | 14HC_CPR_32 | AGAAAGGTTATCTTTTTCTATGGATCTCAGACGGGAACTGCCGAAGACTTCG |
| | | CGTCTAGATTAGCTAAGCAGTGCAGCCAGAAGTATGGCGTGTCTTGCATGAC |
| | | AGCTGATATAGAAATGTACGATCTTAGTTACCTAGATACCTTGTCCGAGGAC |
| | | AGTTTGGTATGTTTTGTCATGGCAACTTACGGAGAAGGTGAGCCTACGGATA |
| | | ATGCAGTAGACTTCTGGGAACAATTTATCACAGACGAATCACCCGTGTTTTC |
| | | AGAGGGAGGAGAAACCCTAGAGAATTTGCGTTATCTAATGTTTGGTTTGGG |
| | | GAATAAAACTTACGAGCACTACAATGCCGTAGCCCGTATTCTTGATAAGAAA |
| | | CTTACCGGTTTGGGGGCTAAGCGTATTGGAGAAAGGGGGGAAGGGGATGAC |
| | | GATGGCTCACTAGAAGAGGACTTTTTGGCGTGGCAAGAGTCTATGTGGCCCA |
| | | CATTTTGTGACGCATTGGGAGTTGATGAAAATAACGCCCAACAGGGGCCCAG |
| | | ACAGGCAAGCTACAGTGTCGATGAGTTAAAAGAGGAGTATAAAAACGATGA |
| | | CGTCTACTTCGGTGAGCTGGGAACCAAGTCAAAGGATTCCTCCAGAGTTGTT |
| | | TATGACGCGAAGCGTCCGTACAACGCCCCAATAACGACGAGAGAGCTATTTA |
| | | ATTCTAGCGAAAGACATTGCCTGCACGTTGACATAGACATCTCAGGAACTAA |
| | | TCTGTCCTATCAAACGGGTGATCACGTGGCCATGTGGCCAACCAACAATGAA |
| | | GATGAAGTGCTGAGATTAGCGAACATCTTGGGACTGCAAGATAAACTGGAC |
| | | AATGTTATAAGCGTTAAGGCCATTGATCCGGCAGCTCCAAAGCAACACCCCT |
| | | TTCCTGTTCCTACCACTTATAGGGCAATTTTTCGTCACTATATTGATATTTG |
| | | CGCGCCCGCTTCAAGGCAAAGTCTGATGAGTTTCGTAGAATTCGCTCCCACA |
| | | GATTCCGCCAAAGACTTACTAAAGCTTCTAGCTACGGATAAAGATGAGTATA |
| | | GGCTGAAGGTGGGTGAGGCCGTGAGGAACCTTGGCGAGGTCCTTGAGTTGGT |
| | | TTCCGGCAATGACAAAGACACACAGCCGGGAAGCTTTACCAGTGTACCGTTT |
| | | GATCTGATAGTTGAGACGATACCTCGTCTTCAACCCAGATATTACTCAATCA |
| | | GTTCCTCTTCCAAGGAAAATTCATCTATTATCTCCGCGACTTGCGTTACGCT |
| | | AGCTTACCAACCGGACCCCACCCCGGATAGAACGGTATATGGTGTGAACACT |
| | | AATTTCCTTTACCGTATTCACACCCAGAGTAGTGACAACGATATCCAAGGGC |
| | | TGCCCAAGTACGACCTAGCGGGGCCGAGAAAAGCCTTTCTGAACGAACAAGG |
| | | CCAATCACACAAACTACCTATTCACATCCGTCGTTCCCAGTTTAAATTGCCA |
| | | AGAAATACTTCCTGTCCGGTGATCATGATTGGTCCCGGGACTGGCGTAGCGC |
| | | CTTTCCGTGGGTTTGTCAGGGAAAGGGCGCTTCAGAAAAAAGAAGGTAAGT |
| | | CCGTTGGGCCAACTGTCCTGTTTTTCGGTAATAGACATAGTGAACACGACTT |
| | | CCTATATTCTGATGAGTGGCCAGAGCTGTTTAATACATTGGGGGACGATTCC |
| | | AAACTTATCACTGCCTTTAGTAGGGAAACAGAACACAAAGTCTACGTACAAC |
| | | ACCGTCTAGAAGAAAATGGGAAAGACATCTGGCAATTGTTAGAAAAGGGTG |
| | | CGTATATTTACGTGTGTGGAGACGCCAGAAACATGGCCCGTGACGTGAACCA |
| | | AACGTTCGTTAATCTAGCGATGGAGTATGGGGAGAAAACTGAGCAGAAGGC |
| | | ACTAGACTATGTGAAGTCTCTTCGTAATACTGGACGTTATCAGGAGGATGTT |
| | | TGGTCATAA |

TABLE 19
Plasmid Sequences

| SEQ ID | | |
|---|---|---|
| NO. | Description | Nucleic Acid Sequence |
| 176 | pA36 (containing | TGTAACTTACACGCGCCTCGTATCTTTTAATGATGGAATAATTTGGGAA |
| | 14HC_P450_5) | TTTACTCTGTGTTTATTTATTTTTATGTTTTGTATTTGGATTTTAGAAA |
| | This plasmid | GTAAATAAAGAAGGTAGAAGAGTTACGGAATGAAGAAAAAAAAATAAA |
| | expresses | CAAAGGTTTAAAAAATTTCAACAAAAAGCGTACTTTACATATATATTTA |
| | 14HC_P450_5 off | TTAGACAAGAAAAGCAGATTAAATAGATATACATTCGATTAACGATAAG |
| | of a TDH3 | TAAAATGTAAAATCACAGGATTTTCGTGTGTGGTCTTCTACACAGACAA |
| | promoter with a | GGTGAAACAATTCGGCATTAATACCTGAGAGCAGGAAGAGCAAGATAAA |
| | CYC1 terminator, | AGGTAGTATTTGTTGGCGATCCCCCTAGAGTCTTTTACATCTTCGGAAAA |
| | contains a | CAAAAACTATTTTTTCTTTAATTTCTTTTTTTACTTTCTATTTTTAATTT |
| | CEN/ARS origin | ATATATTTATATTAAAAAATTTAAATTATAATTATTTTTATAGCACGTG |
| | for replication in | ATGAAAAGGACCCAGGTGGCACTTTTCGGGGAAATGTGCGCGGAACCCCT |
| | S. cerevisiae, a | ATTTGTTTATTTTTCTAAATACATTCAAATATGTATCCGCTCATGAGAC |
| | pBR322 origin | AATAACCCTGATAAATGCTTCAATAATATTGAAAAAGGAAGAGTATGAG |
| | for replication in | TATTCAACATTTCCGTGTCGCCCTTATTCCCTTTTTTGCGGCATTTTGCC |
| | E. coli, a TRP1 | TTCCTGTTTTTGCTCACCCAGAAACGCTGGTGAAAGTAAAAGATGCTGA |
| | auxotrophic | AGATCAGTTGGGACGCGTAGTCTAGACCAGCCAGGACAGAAATGCCTCG |
| | marker, and | ACTTCGCTGCTACCCAAGGTTGCCGGGTGACGCACACCGTGGAAACGGAT |
| | spectinomycin | GAAGGCACGAACCCAGTGGACATAAGCCTGTTCGGTTCGTAAGCTGTAA |
| | resistance. | TGCAAGTAGCGTATGCGCTCACGCAACTGGTCCAGAACCTTGACCGAACG |
| | | CAGCGGTGGTAACGGCGCAGTGGCGGTTTTCATGGCTTGTTATGACTGTT |
| | | TTTTTGGGGTACAGTCTATGCCTCGGGCATCCAAGCAGCAAGCGCGTTAC |
| | | GCCGTGGGTCGATGTTTGATGTTATGGAGCAGCAACGATGTTACGCAGC |
| | | AGGGCAGTCGCCCTAAAACAAAGTTAAACATTATGAGGGAAGCGGTGAT |
| | | CGCCGAAGTATCGACTCAACTATCAGAGGTAGTTGGCGCCATCGAGCGCC |
| | | ATCTCGAACCGACGTTGCTGGCCGTACATTTGTACGGCTCCGCAGTGGAT |
| | | GGCGGCCTGAAGCCACACAGTGATATTGATTTGCTGGTTACGGTGACCGT |
| | | AAGGCTTGATGAAACAACGCGGCGAGCTTTGATCAACGACCTTTTGGAA |
| | | ACTTCGGCTTCCCCTGGAGAGAGCGAGATTCTCCGCGCTGTAGAAGTCAC |
| | | CATTGTTGTGCACGACGACATCATTCCGTGGCGTTATCCAGCTAAGCGCG |
| | | AACTGCAATTTGGAGAATGGCAGCGCAATGACATTCTTGCAGGTATCTT |
| | | CGAGCCAGCCACGATCGACATTGATCTGGCTATCTTGCTGACAAAAGCAA |
| | | GAGAACATAGCGTTGCCTTGGTAGGTCCAGCGGCGGAGGAACTCTTTGA |
| | | TCCGGTTCCTGAACAGGATCTATTTGAGGCGCTAAATGAAACCTTAACGC |
| | | TATGGAACTCGCCGCCCGACTGGGCTGGCGATGAGCGAAATGTAGTGCTT |
| | | ACGTTGTCCCGCATTTGGTACAGCGCAGTAACCGGCAAAATCGCGCCGAA |
| | | GGATGTCGCTGCCGGCTGGGCAATGGAGCGCCTGCCGGCCCAGTATCAGC |
| | | CCGTCATACTTGAAGCTAGACAGGCTTATCTTGGACAAGAAGAAGATCG |
| | | CTTGGCCTCGCGCGCAGATCAGTTGGAAGAATTTGTCCACTACGTGAAAG |
| | | GCGAGATCACCAAGGTAGTCGGCAAATAACCCTCGAGCATTCAAGGCGCC |
| | | TTGATTATTTGACGTGGTTTGATGGCCTCCACGCACGTTGTGATATGTAG |
| | | ATGATTCAGTTCGAGTTTATCATTATCAATACTGCCATTTCAAAGAATA |
| | | CGTAAATAATTAATAGTAGTGATTTTCCTAACTTTATTTAGTCAAAAAA |
| | | TTAGCCTTTTAATTCTGCTGTAACCCGTACATGCCCAAAATAGGGGGCGG |
| | | GTTACACAGAATATATAACATCGTAGGTGTCTGGGTGAACAGTTTATTC |
| | | CTGGCATCCACTAAATATAATGGAGCCCGCTTTTTAAGCTGGCATCCAGA |
| | | AAAAAAAAGAATCCCAGCACCAAAATATTGTTTTCTTCACCAACCATCA |
| | | GTTCATAGGTCCATTCTCTTAGCGCAACTACAGAGAACAGGGGCACAAAC |
| | | AGGCAAAAAACGGGCACAACCTCAATGGAGTGATGCAACCTGCCTGGAG |
| | | TAAATGATGACACAAGGCAATTGACCCACGCATGTATCTATCTCATTTTC |
| | | TTACACCTTCTATTACCTTCTGCTCTCTCTGATTTGGAAAAAGCTGAAAA |
| | | AAAAGGTTGAAACCAGTTCCCTGAAATTATTCCCCTACTTGACTAATAA |
| | | GTATATAAAGACGGTAGGTATTGATTGTAATTCTGTAAATCTATTTCTT |
| | | AAACTTCTTAAATTCTACTTTTATAGTTAGTCTTTTTTTTAGTTTTAAA |
| | | ACACCAAGAACTTAGTTTCGAATAAACACACATAAACAAACAAAATGAC |
| | | AGAGATCAAGGAGCACATCTACAGATACCGTCACTATATCGGGGTTGCG |
| | | GCTGCTGTCGCTTTGGTGTGCCAACAAGTGTATTACAGGATCTTTCGTAT |
| | | CCCTAAGAACCTGAGGCACATACCTGCCATTCCGTACGGTCAACAACTAA |
| | | AGGCATTGAGGTCCGATGAGGCTCTTACCAGCAGGACGAAGCGTTTAGT |
| | | ATTCCCTTTGCTATCTAAGTGTAATGGGGTATACCTTAATAGAATGCCAT |
| | | TTAAGTGGACAATTTATGTCGCGGATCCAGAAATCGCGAGGGCGATTCT |
| | | TTTTAAACCCGAATTCGGCCACAAGACCAGGAGTGTGACAGATTCCTTGG |
| | | ATAAGAACTCAAGCCTATTTGAATTTGTTGGGGACGATAATATAGCCAT |
| | | AGTTAATGGCCATGAATGGAAAGAGCAACGTAAGATAATGAACCCAGCA |
| | | TTCCACAGGGCTACGCCCGTAGGGATGTTCGGCAGTTTAATGCCTAAAGT |
| | | TTTTAGGCTTGTGGAAGAACAGCCTACACTACCCGCCCTAGATTTAATGC |
| | | AAAAGTTAACGTTAGACGCACTTGGGAAGTCAGTATTCGGCTTTGAATT |
| | | CGGCGCACTAGATGACCCCGACAGTGTGTGGGTTAAGACTTATAGACAA |
| | | GTGTTTGACTCTTTTACCGACGTTTTCTCTCTAGTGTTTCCCCGTTTAGA |
| | | TCCAATCTACCGTTATTTTTCTGCGAAACACAGAGAACAGTACAACGCTG |
| | | TTTACAAACTAATTGATCTGCTGGATGGAATGGCAGACAAGAAAAGGAG |
| | | CATGTTGCAAGATGCCTCCAACTCCGATATCAAAGATGTTCCAGACCACG |
| | | AGAAAGATTTACTGCAACTTATGTTGGAGGCTGAACTGCGTGGCGAAGG |
| | | CTCATGGACAAAACGTGAATTGCGTCATAACATGGCCATATTTTTTGTG |
| | | GCAGGTCAGGACACAACGAGTCATGCTTTGACTTTTTGCCTTTATTTACT |
| | | TGCGAAGAATCAAGATATCCAGAAGAAGGCGAGAGAAGAAATCTTAAAT |
| | | GTTTTTGGGGATGAACCTAAAGACGTGTTTCCCACACTTGAAGATTGCA |
| | | AAAAACTAAATTATCTTGACATGGTGATCAAGGAATCCATGAGAATTTA |
| | | CCCTCCTGCTAACGATGTCCTAGCCAGGGATGTCAATGAAGATTTAAACG |
| | | TCAAGGGTGTCTTCATTCCCAAGGGATCTATGGTATCAGTGGACATTCAT |
| | | GCCCTACACCATAGACCAGACCTGTGGCATGAGCCCGATAAGTTCAATCC |
| | | CGATCGTTTCCTGCCGGGTGGGGAGCATGACAGTCACGTCGGCGTAACAT |
| | | ACGCCCCCTTCTCATCCGGCTCCAGACAGTGCATCGCGTTAAAATTCGCT |
| | | ACCATGCAGCAAAGGGTAGTCCTGTCCATGTTGCTGAGAAAGTACGAAT |
| | | GGGAATTGCCAAAGGATTCCAAACATAAGGACTCCATCCAGTTCCAGAT |
| | | TCCCTTCAACATCGCTCCTAAGGATCTAGAGCTTACCTTTCATAAACGTT |
| | | ACTAGACAGGCCCCTTTTCCTTTGTCGATATCATGTAATTAGTTATGTCA |
| | | CGCTTACATTCACGCCCTCCCCCCACATCCGCTCTAACCGAAAAGGAAGG |
| | | AGTTAGACAACCTGAAGTCTAGGTCCCTATTTATTTTTTTATAGTTATGT |
| | | TAGTATTAAGAACGTTATTTATATTTCAAATTTTTCTTTTTTTTCTGTAC |
| | | AAACGCGTGTACGCATGTAACATTATACTGAAAACCTTGCTTGAGAAGG |
| | | TTTTGGGACGCTCGAAGGCTTTAATTTGTAATCATTATCACTTTACGGGT |
| | | CCTTTCCGGTGATCCGACAGGTTACGGGGCGGCGACCTCGCGGGTTTTCG |
| | | CTATTTATGAAAATTTTCCGGTTTAAGGCGTTTCCGTTCTTCTTCGTCAT |
| | | AACTTAATGTTTTTATTTAAAATACCTCGCGAGTGGCAACACTGAAAAT |
| | | ACCCATGGAGCGGCGTAACCGTCGCACAGGATCTAGGTGAAGATCCTTTT |
| | | TGATAATCTCATGACCAAAATCCCTTAACGTGAGTTTTCGTTCCACTGAG |
| | | CGTCAGACCCCGTAGAAAAGATCAAAGGATCTTCTTGAGATCCTTTTTTT |
| | | CTGCGCGTAATCTGCTGCTTGCAAACAAAAAAACCACCGCTACCAGCGGT |
| | | GGTTTGTTTGCCGGATCAAGAGCTACCAACTCTTTTTCCGAAGGTAACTG |
| | | GCTTCAGCAGAGCGCAGATACCAAATACTGTCCTTCTAGTGTAGCCGTAG |
| | | TTAGGCCACCACTTCAAGAACTCTGTAGCACCGCCTACATACCTCGCTCT |
| | | GCTAATCCTGTTACCAGTGGCTGCTGCCAGTGGCGATAAGTCGTGTCTTA |
| | | CCGGGTTGGACTCAAGACGATAGTTACCGGATAAGGCGCAGCGGTCGGGC |
| | | TGAACGGGGGGTTCGTGCACACAGCCCAGCTTGGAGCGAACGACCTACAC |
| | | CGAACTGAGATACCTACAGCGTGAGCTATGAGAAAGCGCCACGCTTCCCG |
| | | AAGGGAGAAAGGCGGACAGGTATCCGGTAAGCGGCAGGGTCGGAACAGG |
| | | AGAGCGCACGAGGGAGCTTCCAGGGGGAAACGCCTGGTATCTTTATAGT |
| | | CCTGTCGGGTTTCGCCACCTCTGACTTGAGCGTCGATTTTTGTGATGCTC |
| | | GTCAGGGGGGCGGAGCCTATGGAAAAACGCCAGCAACGCGGCAGTGGAA |
| | | CGTGCATTATGAATTAGTTACGCTAGGGATAACAGGGTAATATAGAACC |
| | | CGAACGACCGAGCGCAGCGGCGGCCGCGCTGATACCGCCGCCCTCGCCGC |
| | | AGTTAATTAAAGTCAGTGAGCGAGGAAGCGCGTAACTATAACGGTCCTA |
| | | AGGTAGCGAATCCTGATGCGGTATTTTCTCCTTACGCATCTGTGCGGTAT |
| | | TTCACACCGCATAGATCGGCAAGTGCACAAACAATACTTAAATAAATAC |
| | | TACTCAGTAATAACCTATTTCTTAGCATTTTTGACGAAATTTGCTATTTT |
| | | GTTAGAGTCTTTTACACCATTTGTCTCCACACCTCCGCTTACATCAACAC |
| | | CAATAACGCCATTTAATCTAAGCGCATCACCAACATTTTCTGGCGTCAGT |
| | | CCACCAGCTAACATAAAATGTAAGCTTTCGGGGCTCTCTTGCCTTCCAAC |
| | | CCAGTCAGAAATCGAGTTCCAATCCAAAAGTTCACCTGTCCCACCTGCTT |
| | | CTGAATCAAACAAGGGAATAAACGAATGAGGTTTCTGTGAAGCTGCACT |
| | | GAGTAGTATGTTGCAGTCTTTTGGAAATACGAGTCTTTTAATAACTGGC |
| | | AAACCGAGGAACTCTTGGTATTCTTGCCACGACTCATCTCCATGCAGTTG |
| | | GACGATATCAATGCCGTAATCATTGACCAGAGCCAAAACATCCTCCTTAA |
| | | GTTGATTACGAAACACGCCAACCAAGTATTTCGGAGTGCCTGAACTATTT |
| | | TTATATGCTTTTACAAGACTTGAAATTTTCCTTGCAATAACCGGGTCAAT |
| | | TGTTCTCTTTCTATTGGGCACACATATAATACCCAGCAAGTCAGCATCGG |
| | | AATCTAGAGCACATTCTGCGGCCTCTGTGCTCTGCAAGCCGCAAACTTTC |
| | | ACCAATGGACCAGAACTACCTGTGAAATTAATAACAGACATACTCCAAG |
| | | CTGCCTTTGTGTGCTTAATCACGTATACTCACGTGCTCAATAGTCACCAA |
| | | TGCCCTCCCTCTTGGCCCTCTCCTTTTCTTTTTTCGACCGAATTAATTCTT |
| | | AATCGGCAAAAAAAGAAAAGCTCCGGATCAAGATTGTACGTAAGGTGAC |
| | | AAGCTATTTTTCAATAAAGAATATCTTCCACTACTGCCATCTGGCGTCAT |
| | | AACTGCAAAGTACACATATATTACGATGCTGTTCTATTAAATGCTTCCTA |
| | | TATTATATATATAGTAATGTCGTGATCTATGGTGCACTCTCAGTACAAT |
| | | CTGCTCTGATGCCGCATAGTTAAGCCAGCCCCGACACCCGCCAACACCCG |
| | | CTGACGCGCCCTGACGGGCTTGTCTGCTCCCGGCATCCGCTTACAGACAA |
| | | GCTGTGACCGTCTCCGGGAGCTGCATGTGTCAGAGGTTTTCACCGTCATC |
| | | ACCGAAACGCGCGAGACGAAAGGGCCTCGTGATACGCCTATTTTTATAGG |
| | | TTAATGTCATGATAATAATGGTTTCTTAGACGGATCGCTTGCC |
| 177 | pA48 | AACGACCTACACCGAACTGAGATACCTACAGCGTGAGCTATGAGAAAGC |
| | (containing | GCCACGCTTCCCGAAGGGAGAAAGGCGGACAGGTATCCGGTAAGCGGCA |
| | 14HC_P450_5) | GGGTCGGAACAGGAGAGCGCACGAGGGAGCTTCCAGGGGGAAACGCCTG |
| | This plasmid | GTATCTTTATAGTCCTGTCGGGTTTCGCCACCTCTGACTTGAGCGTCGAT |
| | expresses | TTTTGTGATGCTCGTCAGGGGGGCGGAGCCTATGGAAAAACGCCAGCAA |
| | 14HC_CPR_1 off | CGCGGCAGTGGAACGTGCATTATGAATTAGTTACGCTAGGGATAACAGG |
| | of a TDH3 | GTAATATAGAACCCGAACGACCGAGCGCAGCGGCGGCCGCGCTGATACCG |
| | promoter with a | CCGCCCTCGCCGCAGTTAATTAAAGTCAGTGAGCGAGGAAGCGCGTAACT |
| | CYC1 terminator, | ATAACGGTCCTAAGGTAGCGAATCCTGATGGGTAATAACTGATATAATT |
| | contains a | AAATTGAAGCTCTAATTTGTGAGTTTAGTATACATGCATTTACTTATAA |
| | CEN/ARS origin | TACAGTTTTTTAGTTTTGCTGGCCGCATCTTCTCAAATATGCTTCCCAGC |
| | for replication in | CTGCTTTTCTGTAACGTTCACCCTCTACCTTAGCATCCCTTCCCTTTGCA |
| | S. cerevisiae, a | AATAGTCCTCTTCCAACAATAATAATGTCAGATCCTGTAGAGACCACATC |
| | pBR322 origin | ATCCACGGTTCTATACTGTTGACCCAATGCGTCTCCCTTGTCATCTAAAC |
| | for replication in | CCACACCGGGTGTCATAATCAACCAATCGTAACCTTCATCTCTTCCACCC |
| | E. coli, a URA3 | ATGTCTCTTTGAGCAATAAAGCCGATAACAAAATCTTTGTCGCTCTTCGC |
| | auxotrophic | AATGTCAACAGTACCCTTAGTATATTCTCCAGTAGATAGGGAGCCCTTGC |
| | marker, and | ATGACAATTCTGCTAACATCAAAAGGCCTCTAGGTTCCTTTGTTACTTCT |
| | spectinomycin | TCTGCCGCCTGCTTCAAACCGCTAACAATACCTGGGCCCACCACACCGTG |
| | resistance | TGCATTCGTAATGTCTGCCCATTCTGCTATTCTGTATACACCCGCAGAGT |
| | | ACTGCAATTTGACTGTATTACCAATGTCAGCAAATTTTCTGTCTTCGAAG |
| | | AGTAAAAAATTGTACTTGGCGGATAATGCCTTTAGCGGCTTAACTGTGC |
| | | CCTCCATGGAAAAATCAGTCAAGATATCCACATGTGTTTTTAGTAAACA |
| | | AATTTTGGGACCTAATGCTTCAACTAACTCCAGTAATTCCTTGGTGGTAC |
| | | GAACATCCAATGAAGCACACAAGTTTGTTTGCTTTTCGTGCATGATATT |
| | | AAATAGCTTGGCAGCAACAGGACTAGGATGAGTAGCAGCACGTTCCTTA |
| | | TATGTAGCTTTCGACATGATTTATCTTCGTTTCCTGCAGGTTTTTGTTCT |
| | | GTGCAGTTGGGTTAAGAATACTGGGCAATTTCATGTTTCTTCAACACTAC |
| | | ATATGCGTATATATACCAATCTAAGTCTGTGCTCCTTCCTTCGTTCTTCC |
| | | TTCTGTTCGGAGATTACCGAATCAAAAAAATTTCAAAGAAACCGAAATC |
| | | AAAAAAAAGAATAAAAAAAAAATGATGAATTGAATTGAAAAGCTTCCT |
| | | ATATTATATATATAGTAATGTCGTGATCTATGGTGCACTCTCAGTACAA |
| | | TCTGCTCTGATGCCGCATAGTTAAGCCAGCCCCGACACCCGCCAACACCC |
| | | GCTGACGCGCCCTGACGGGCTTGTCTGCTCCCGGCATCCGCTTACAGACA |
| | | AGCTGTGACCGTCTCCGGGAGCTGCATGTGTCAGAGGTTTTCACCGTCAT |
| | | CACCGAAACGCGCGAGACGAAAGGGCCTCGTGATACGCCTATTTTTATAG |
| | | GTTAATGTCATGATAATAATGGTTTCTTAGACGGATCGCTTGCCTGTAA |
| | | CTTACACGCGCCTCGTATCTTTTAATGATGGAATAATTTGGGAATTTACT |
| | | CTGTGTTTATTTATTTTTATGTTTTGTATTTGGATTTTAGAAAGTAAAT |
| | | AAAGAAGGTAGAAGAGTTACGGAATGAAGAAAAAAAAATAAACAAAGG |
| | | TTTAAAAAATTTCAACAAAAAGCGTACTTTACATATATATTTATTAGAC |
| | | AAGAAAAGCAGATTAAATAGATATACATTCGATTAACGATAAGTAAAAT |
| | | GTAAAATCACAGGATTTTCGTGTGTGGTCTTCTACACAGACAAGGTGAA |
| | | ACAATTCGGCATTAATACCTGAGAGCAGGAAGAGCAAGATAAAAGGTAG |
| | | TATTTGTTGGCGATCCCCCTAGAGTCTTTTACATCTTCGGAAAACAAAAA |
| | | CTATTTTTTCTTTAATTTCTTTTTTTACTTTCTATTTTTAATTTATATAT |
| | | TTATATTAAAAAATTTAAATTATAATTATTTTTATAGCACGTGATGAAA |
| | | AGGACCCAGGTGGCACTTTTCGGGGAAATGTGCGCGGAACCCCTATTTGT |
| | | TTATTTTTCTAAATACATTCAAATATGTATCCGCTCATGAGACAATAACC |
| | | CTGATAAATGCTTCAATAATATTGAAAAAGGAAGAGTATGAGTATTCAA |
| | | CATTTCCGTGTCGCCCTTATTCCCTTTTTTGCGGCATTTTGCCTTCCTGT |
| | | TTTTGCTCACCCAGAAACGCTGGTGAAAGTAAAAGATGCTGAAGATCAG |
| | | TTGGGACGCGTAGTCTAGACCAGCCAGGACAGAAATGCCTCGACTTCGCT |
| | | GCTACCCAAGGTTGCCGGGTGACGCACACCGTGGAAACGGATGAAGGCAC |
| | | GAACCCAGTGGACATAAGCCTGTTCGGTTCGTAAGCTGTAATGCAAGTA |
| | | GCGTATGCGCTCACGCAACTGGTCCAGAACCTTGACCGAACGCAGCGGTG |
| | | GTAACGGCGCAGTGGCGGTTTTCATGGCTTGTTATGACTGTTTTTTTGGG |
| | | GTACAGTCTATGCCTCGGGCATCCAAGCAGCAAGCGCGTTACGCCGTGGG |
| | | TCGATGTTTGATGTTATGGAGCAGCAACGATGTTACGCAGCAGGGCAGT |
| | | CGCCCTAAAACAAAGTTAAACATTATGAGGGAAGCGGTGATCGCCGAAG |
| | | TATCGACTCAACTATCAGAGGTAGTTGGCGCCATCGAGCGCCATCTCGAA |
| | | CCGACGTTGCTGGCCGTACATTTGTACGGCTCCGCAGTGGATGGCGGCCT |
| | | GAAGCCACACAGTGATATTGATTTGCTGGTTACGGTGACCGTAAGGCTT |
| | | GATGAAACAACGCGGCGAGCTTTGATCAACGACCTTTTGGAAACTTCGGC |
| | | TTCCCCTGGAGAGAGCGAGATTCTCCGCGCTGTAGAAGTCACCATTGTTG |
| | | TGCACGACGACATCATTCCGTGGCGTTATCCAGCTAAGCGCGAACTGCAA |
| | | TTTGGAGAATGGCAGCGCAATGACATTCTTGCAGGTATCTTCGAGCCAGC |
| | | CACGATCGACATTGATCTGGCTATCTTGCTGACAAAAGCAAGAGAACAT |
| | | AGCGTTGCCTTGGTAGGTCCAGCGGCGGAGGAACTCTTTGATCCGGTTCC |
| | | TGAACAGGATCTATTTGAGGCGCTAAATGAAACCTTAACGCTATGGAAC |
| | | TCGCCGCCCGACTGGGCTGGCGATGAGCGAAATGTAGTGCTTACGTTGTC |
| | | CCGCATTTGGTACAGCGCAGTAACCGGCAAAATCGCGCCGAAGGATGTCG |
| | | CTGCCGGCTGGGCAATGGAGCGCCTGCCGGCCCAGTATCAGCCCGTCATA |
| | | CTTGAAGCTAGACAGGCTTATCTTGGACAAGAAGAAGATCGCTTGGCCTC |
| | | GCGCGCAGATCAGTTGGAAGAATTTGTCCACTACGTGAAAGGCGAGATC |
| | | ACCAAGGTAGTCGGCAAATAACCCTCGAGCATTCAAGGCGCCTTGATTAT |
| | | TTGACGTGGTTTGATGGCCTCCACGCACGTTGTGATATGTAGATGATTCA |
| | | GTTCGAGTTTATCATTATCAATACTGCCATTTCAAAGAATACGTAAATA |
| | | ATTAATAGTAGTGATTTTCCTAACTTTATTTAGTCAAAAAATTAGCCTT |
| | | TTAATTCTGCTGTAACCCGTACATGCCCAAAATAGGGGGCGGGTTACACA |
| | | GAATATATAACATCGTAGGTGTCTGGGTGAACAGTTTATTCCTGGCATC |
| | | CACTAAATATAATGGAGCCCGCTTTTTAAGCTGGCATCCAGAAAAAAAA |
| | | AGAATCCCAGCACCAAAATATTGTTTTCTTCACCAACCATCAGTTCATAG |
| | | GTCCATTCTCTTAGCGCAACTACAGAGAACAGGGGCACAAACAGGCAAA |
| | | AAACGGGCACAACCTCAATGGAGTGATGCAACCTGCCTGGAGTAAATGA |
| | | TGACACAAGGCAATTGACCCACGCATGTATCTATCTCATTTTCTTACACC |
| | | TTCTATTACCTTCTGCTCTCTCTGATTTGGAAAAAGCTGAAAAAAAAGG |
| | | TTGAAACCAGTTCCCTGAAATTATTCCCCTACTTGACTAATAAGTATATA |
| | | AAGACGGTAGGTATTGATTGTAATTCTGTAAATCTATTTCTTAAACTTC |
| | | TTAAATTCTACTTTTATAGTTAGTCTTTTTTTTAGTTTTAAAACACCAA |
| | | GAACTTAGTTTCGAATAAACACACATAAACAAACAAAATGGCTCAATCT |
| | | CCACCAGCATTGGATACTTTGGATATCGTTTTCTTGGGTACTATCGGTTT |
| | | GGGTACAATTGCTTGGTTTGCTAGAAGGCAAATTGCTGAAAGATTATTC |
| | | GGTTCCACCTCTTCCTCTGATGCTAAATCTAATGGTCATGCTACTCCAGC |
| | | TCCACCAAAAAGAGAAAGAAACTTCGTTAAGATCATGCAAGAGCAAGGT |
| | | AGAAAGGTCATTTTCTTCTACGGTTCTCAAACTGGTACTGCTGAAGATT |
| | | ACGCTTCTAGATTGGCTAAAGAATGCTCTCAAAAGTACGGTGTTAACTG |
| | | TATGACTGCTGATTTGGAGTTGTACGACTTGTCTTACTTGGATACTGTTC |
| | | CAGAAGATTGCCTGGTTTTTTTCGTTATGGCTACTTATGGTGAAGGTGA |
| | | ACCTACTGATAATGCTGTTGATTTCTGGGATGTCTTGTCTGAAGAAGAA |
| | | CCACAATTTTCTGAAGCCGAAGGTGATAAGCCATTGCAAAATTTGAGAT |
| | | ACTTGGTTTTCGGCTTGGGTAACAAGACTTACGAACATTATAACGCTGTT |
| | | GCCAGAAACGTTGACAAGAGATTGGAAGTTTTGGGTGCTCATAGAATTC |
| | | ATGAACGTGGTGAGGGTGATGATGATGGTTCTTTGGAAGAAGATTTTTT |
| | | GGCCTGGCAAGAAAACATGTGGCCAGCTTTTTGTGAAGCTTTGGGTGTT |
| | | GATGAATCTAACGCTCATTCTGGTCCAAGACAAGCTACTTATTCTGTTGA |
| | | AGAATTGGTCGATGTCAACATGGATGATGTTTACTTGGGTGAATTGGCC |
| | | GAAAAACCTAAAGAAGGTGCTAGAGTTATCTACGATGCTAAGAGGCCTT |
| | | TTAATGCTCCAATTGCCATTTCTCAAGACTTGTTCACTAACACCGATAGA |
| | | CATTGCTTGCACATGGAAATCGATATCTCCGATTCCAACTTGTCATACCA |
| | | AACCGGTGATCATATTGCTATTTGGCCAACTAACTCCGAAAACGAAGTT |
| | | GCTAGACTGGCTTCTATTTTGGGTTTAGCTGATAAGTTGGATACCGCCAT |
| | | CAATGTTAAGGCTATTGATCCAGCTGCTTCCAAAAAGTACCCATTTCCTT |
| | | GTCCAGCTACTTACAGAGCTATTTTCAGACATTACTTGGACATTTGCGCT |
| | | GCCGTTTCTAGACAATCTTTGATGGCTTTTGTTGAATACGCTCCAACCGA |
| | | AGAATCCAAAGAAAGATTGAGACAATTGGCCAAGGATAAGGATGAGTAC |
| | | AGATTGACTGTTGGTGAAGCTGTTAGAAATTTGGGTGAAGTATTGGAAA |
| | | TCGTTGCTGGTAATGATGTTAAGCCAGGTTTCTTTTCTTCCGTTCCATTC |
| | | GATTTGGTTGTCGAATCTATTTCCAGATTGCAACCTAGGTACTACTCCAT |
| | | TTCTTCATCTGCAAAAGAGTCCCCAAAAAAGATCGCTGTTACTGCTGTTA |
| | | CATTGTCCTATCAACCAGATCCAACTCCACAAAGAACTGTTTATGGTGTC |
| | | AACACTAATTACTTGTGGCGTATTCATACCGCCTCTAAGCAACAATCTAC |
| | | TGAAGCTGATTTGCCAACCTATGATTTGTCAGGTCCAAGAAATGCTTTAC |
| | | ATGGTACTAAGTTGCCAGTTCACGTTAGAAGATCTCAATTCAAGTTGCC |
| | | AAGAAACCCAACCGTTCCAGTTATTATGGTTGGTCCAGGTACTGGTGTTG |
| | | CTCCTTTTAGAGGTTTTGTTAGAGAAAGAGCCTTGCAGAAATCTGAAGG |
| | | TAAACCAGTTGGTCCAACTTTGTTGTTTTTCGGTTGTAGAAACTCCGAAC |
| | | AGGACTTCTTGTACAAAGATGAATGGCCTGCTTTGTTTGACACCTTAGGT |
| | | GAATCCTCTAGAATTATTACCGCCTTCTCAAGAGAAACCGCTCAAAAAGT |
| | | TTACGTCCAGCATAGATTGCAAGAGAACGGTCAAGAAGTTTGGGGTTTG |
| | | TTGCAAAGGGGTGCTTATATCTATGTTTGTGGTGATGCAAAGAACATGG |
| | | CCAGAGATGTTCAACAAACTTTCGTTAACTTCGGTATCGAATTCGGTGGT |
| | | TTGTCTGATGATAAGGCTCATGATTTTGTCAAGAACTTGAGAAACACCG |
| | | GTAGATACCAAGAAGATGTTTGGTCTTGAACAGGCCCCTTTTCCTTTGTC |
| | | GATATCATGTAATTAGTTATGTCACGCTTACATTCACGCCCTCCCCCCAC |
| | | ATCCGCTCTAACCGAAAAGGAAGGAGTTAGACAACCTGAAGTCTAGGTC |
| | | CCTATTTATTTTTTTATAGTTATGTTAGTATTAAGAACGTTATTTATAT |
| | | TTCAAATTTTTCTTTTTTTTCTGTACAAACGCGTGTACGCATGTAACATT |
| | | ATACTGAAAACCTTGCTTGAGAAGGTTTTGGGACGCTCGAAGGCTTTAA |
| | | TTTGTAATCATTATCACTTTACGGGTCCTTTCCGGTGATCCGACAGGTTA |
| | | CGGGGCGGCGACCTCGCGGGTTTTCGCTATTTATGAAAATTTTCCGGTTT |
| | | AAGGCGTTTCCGTTCTTCTTCGTCATAACTTAATGTTTTTATTTAAAATA |
| | | CCTCGCGAGTGGCAACACTGAAAATACCCATGGAGCGGCGTAACCGTCGC |
| | | ACAGGATCTAGGTGAAGATCCTTTTTGATAATCTCATGACCAAAATCCCT |
| | | TAACGTGAGTTTTCGTTCCACTGAGCGTCAGACCCCGTAGAAAAGATCAA |
| | | AGGATCTTCTTGAGATCCTTTTTTTCTGCGCGTAATCTGCTGCTTGCAAA |
| | | CAAAAAAACCACCGCTACCAGCGGTGGTTTGTTTGCCGGATCAAGAGCTA |
| | | CCAACTCTTTTTCCGAAGGTAACTGGCTTCAGCAGAGCGCAGATACCAAA |
| | | TACTGTCCTTCTAGTGTAGCCGTAGTTAGGCCACCACTTCAAGAACTCTG |
| | | TAGCACCGCCTACATACCTCGCTCTGCTAATCCTGTTACCAGTGGCTGCT |
| | | GCCAGTGGCGATAAGTCGTGTCTTACCGGGTTGGACTCAAGACGATAGTT |
| | | ACCGGATAAGGCGCAGCGGTCGGGCTGAACGGGGGGTTCGTGCACACAGC |
| | | CCAGCTTGGAGCG |

While certain embodiments of the disclosure have been shown and described herein, those skilled in the art will appreciate that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of producing a benzylisoquinoline alkaloid (BIA) product in an engineered host cell, the method comprising:
(a) expressing a heterologous enzyme having 14-hydroxylase activity in the engineered host cell;
(b) expressing a heterologous enzyme having cytochrome P450 reductase (CPR) activity in the engineered host cell,
(c) contacting the heterologous enzyme with a BIA-precursor substrate that is a morphinan alkaloid with a free hydrogen at carbon C-14, wherein the heterologous enzyme hydroxylates the C-14 carbon on the BIA-precursor substrate; and
(d) producing the BIA product within the host cell;
wherein the engineered host cell produces more BIA product than a non-engineered host cell.

2. The method of claim 1, wherein the enzyme is a cytochrome P450.

3. The method of claim 1 or claim 2, wherein the heterologous enzyme comprises an amino acid sequence having at least 90%, 95%, 96%, 97%, 98% or 99% sequence identity to SEQ ID NO. 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, or 166.

4. The method of any one of claims 1-3, wherein the BIA-precursor substrate is selected from the group consisting of codeine, codeinone, norcodeinone, hydrocodone, northebaine, oripavine, morphinone, normorphinone, hydromorphine, norhydromorphine, and norhydrocodone.

5. The method of any one of claims 1-4, wherein the BIA product is noroxymorphone.

6. The method of any one of claims 1-5, wherein the engineered host cell expresses a cytochrome P450 reductase (CPR).

7. The method of claim 6, wherein the CPR is heterologous to the engineered host cell.

8. The method of claim 6 or claim 7, wherein the CPR comprises an amino acid sequence having at least 90%, 95%, 96%, 97%, 98% or 99% sequence identity to SEQ ID NO. 168, 170, 172, or 174.

9. The method of one of claims 6-8, wherein the CPR is a fungal CPR.

10. A host cell that produces a BIA product, the host cell comprising a first heterologous polynucleotide that encodes a heterologous enzyme having 14-hydroxylase activity and a second heterologous polynucleotide that encodes a cytochrome P450 reductase (CPR).

11. The host cell of claim 10, wherein the enzyme having 14-hydroxylase is a cytochrome P450.

12. The host cell of claim 10 or claim 11, wherein the enzyme having 14-hydroxylase activity comprises an amino acid sequence having at least 90%, 95%, 96%, 97%, 98% or 99% sequence identity to SEQ ID NO. 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, or 166.

13. The host cell of any one of claims 10-12, wherein the CPR is a fungal CPR.

14. The host cell of any one of claims 10-13, wherein the CPR comprises an amino acid sequence having at least 90%, 95%, 96%, 97%, 98% or 99% sequence identity to SEQ ID NO. 168, 170, 172, or 174.

15. The host cell of any one of claims 10-14, wherein the production of the BIA product comprises hydroxylation of a C-14 carbon on a BIA-precursor substrate.

16. A vector comprising a first polynucleotide that encodes an enzyme having 14-hydroxylase activity and a second polynucleotide that encodes a cytochrome P450 reductase (CPR).

17. The vector of claim 16, wherein the enzyme having 14-hydroxylase is a cytochrome P450.

18. The vector of claim 16 or claim 17, wherein the polynucleotide that encodes the enzyme having 14-hydroxylase activity comprises a nucleotide sequence selected from SEQ ID Nos. 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, and 167.

19. The vector of any one of claims 16-18, wherein the CPR is a fungal CPR.

20. The vector of any one of claims 16-19, wherein the polynucleotide that encodes the CPR comprises a nucleotide sequence selected from SEQ ID Nos. 169, 171, 173, and 175.

21. The vector of any one of claims 16-20, wherein enzyme having 14-hydroxylase activity comprises an amino acid sequence having at least 90%, 95%, 96%, 97%, 98% or 99% sequence identity to SEQ ID NO. 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, or 167.

22. The vector of any one of claims 16-21, wherein the CPR comprises an amino acid sequence having at least 90%, 95%, 96%, 97%, 98% or 99% sequence identity to SEQ ID NO. 169, 171, 173, or 175.

23. A host cell comprising a vector that comprises a first polynucleotide that encodes an enzyme having 14-hydroxylase activity and a second polynucleotide that encodes a cytochrome P450 reductase (CPR).

24. The host cell of claim 23, wherein the enzyme having 14-hydroxylase is a cytochrome P450.

25. The host cell of claim 23 or claim 24, wherein the CPR is a fungal CPR.

26. The host cell of any one of claims 23-25, wherein the polynucleotide that encodes the enzyme having 14-hydroxylase activity comprises a nucleotide sequence selected from SEQ ID Nos. 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, and 167.

27. The host cell of any one of claims 23-26, wherein the polynucleotide that encodes the CPR comprises a nucleotide sequence selected from SEQ ID Nos. 169, 171, 173, and 175.

28. The host cell of any one of claims 23-27, wherein enzyme having 14-hydroxylase activity comprises an amino acid sequence having at least 90%, 95%, 96%, 97%, 98% or 99% sequence identity to SEQ ID NO. 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, or 166.

29. The host cell of any one of claims 23-28, wherein the CPR comprises an amino acid sequence having at least 90%, 95%, 96%, 97%, 98% or 99% sequence identity to SEQ ID NO. 168, 170, 172, or 174.

30. A host cell comprising a vector that comprises a first heterologous polynucleotide sequence that encodes a heterologous enzyme having formaldehyde dehydrogenase activity and a second heterologous polynucleotide designed to repress expression of a target gene, wherein the target gene is selected from DUG2 or DUG3.

31. The host cell of claim 30, wherein the heterologous enzyme is SFA1.


SEQUENCE LISTING (267 sequences)

SEQ ID NO: 1 - protein, 901 aa, Papaver somniferum
  REGION 1..901: note = full-length amino acid sequence: RQNK-2062398
  REGION 1..901: note = full-length amino acid sequence: RQNK-2062398
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNHGNYTTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 2 - protein, 919 aa, Papaver somniferum
  REGION 1..919: note = full-length amino acid sequence: KKCW-2026866
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNHGNYTTXLLLPQLAWRQPWKLYYXTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 3 - protein, 732 aa, Papaver somniferum
  REGION 1..732: note = partial-length amino acid sequence: SUFP-2025636
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNHGNYTTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRXVSKPWLH

SEQ ID NO: 4 - protein, 764 aa, Papaver somniferum
  REGION 1..764: note = partial-length amino acid sequence: MIKW-2013651
MRWHRXIDSYGLSSVPYGKYWRELRKVCVHNLLSNQQLLKFRHLIISQVDTSFNKLYELCKNSEDNQGNYPTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 5 - protein, 900 aa, Papaver setigerum
  REGION 1..900: note = full-length amino acid sequence: EPRK-2027940
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 6 - protein, 724 aa, Papaver setigerum
  REGION 1..724: note = partial-length amino acid sequence: QCOU-2000833
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQALYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEE

SEQ ID NO: 7 - protein, 889 aa, Papaver bracteatum
  REGION 1..889: note = full-length amino acid sequence: SSDU-2015634
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSSKKLQELMATANIPPAVNQVEMSPAFQQKKLREYCNANNILVSAVSILGSNGTPWGSNAVLGSEVLKQIAMAKGKSVAQVSMRWVYEQGASLVVKSFSEERLRENLNIFDWELTKEDNEKIGEIPQCRILTAYFLVSPNGPFKSQEELWDDKA

SEQ ID NO: 8 - protein, 889 aa, Papaver bracteatum
  REGION 1..889: note = full-length amino acid sequence: TMWO-2027322
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSCKKLQELMATANIPPAVNQVEMSPAFQQKKLREYCNANNILVSAVSILGSNGTPWGSNAVLGSEVLKQIAMAKGKSVAQVSMRWVYEQGASLVVKSFSEERLRENLNIFDWELTKEDNEKIGEIPQCRILTAYFLVSPNGPFKSQEELWDDKA

SEQ ID NO: 9 - protein, 848 aa, Papaver bracteatum
  REGION 1..848: note = partial-length amino acid sequence: pbr.PBRST1PF_89405
SSPASSTETAVLCHQRQQSCALPISGLLHIFMNKNGLIHVTLGNMADKYGPIFSFPTGSHRILVVSSWEMVKECFTGNNDTAFSNRPIPLAFKTIFYACRGIDSYGLSSVPYGKYWRELRKVCVHNLLSNQQLLKFRHLIISQVDTSFNKLYELCKNSEDNQGMVRMDDWLAQLSFSVIGRIVCGFQSDPKTGAPSRVEQFKEAINEASYFMSTSPVSDNVPMLGWIDQLTGLTRNMTHCGKKLDLVVESIINDHRQKRRFSRTKGGDEKDDEQDDFIDICLSIMEQPQLPGNNNPPKIPIKSIVLDMIGAGTDTTKLTIIWTLSLLLNNPNVLAKAKQEVDAHFETKKRSTNEASVVVDFDDIGNLVYIQAIIKESMRLYPVSPVVERLSSEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 10 - protein, 847 aa, Papaver bracteatum
  REGION 1..847: note = partial-length amino acid sequence: pbr.PBRST1PF_4328
SSPASSTETAVLCHQRQQSCALPISGLLHIFMNKNGLIHVTLGNMADKYGPIFSFPTGSHRILVVSSWEMVKECFTGNNDTFFSNRPIPLAFKIIFYAGGVDSYGLALVPYGKYWRELRKICVHNLLSNQQLLKFRHLIISQVDTSFNKLYELCKNSEDNQGMVRMDDWLAQLSFSVIGRIVCGFQSDPKTGAPSRVEQFKEAINEASYFMSTSPVSDNVPMLGWIDQLTGLTRNMTHCGKKLDLVVESIINDHRQKRRFSRTKGGDEKDDEQDDFIDICLSIMEQPQLPGNNNPPKIPIKSIVLDMIGGGTDTTKLTTIWTLSLLLNNPHVLDKAKQEVDAHFLTKRRSTNDAAVVDFDDIRNLVYIQAIIKESMRLYPASPVVERLSGEDCVVGGFHVPAGTRLWVNVWKMQRDPNVWADPMVFRPERFLSHGQKKMVDVRGKNYELLPFGAGRRICPGISFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 11 - protein, 845 aa, Papaver bracteatum
  REGION 1..845: note = partial-length amino acid sequence: pbr.PBRST1PF_12180
SSPASSTETAVLCHQRQQSCALPISGLLHIFMNKNGLIHVTLGNMADKYGPIFSFPTGSHRILVVSSWEMVKECFTGNNDTFFSNRPIPLAFKIIFYAGGVDSYGLALVPYGKYWRELRKICVHNLLSNQQLLNFRHLIISQVDTSFNKLYDLSNKKKNTTTDSGTVRMDDWLAQLSFNVIGRIVCGFQTHTETSATSSVERFTEAIDEASRFMSIATVSDTFPWLGWIDQLTGLTRKMKHYGKKLDLVVESIIEDHRQNRRISGTKQGDDFIDICLSIMEQPQIIPGNNDPPRQIPIKSIVLDMIGGGTDTTKLTTTWTLSLLLNNPHVLEKAREEVDAHFGTKRRPTNDDAVMVEFDDIRNLVYIQAIIKESMRLYPASPVVERLSGEDCVVGGFHVPAGTRLWVNVWKMQRDPNVWADPMVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 12 - protein, 869 aa, Papaver bracteatum
  REGION 1..869: note = partial-length amino acid sequence: pbr.PBRST1PF_4329
VALRKKILKNYYSSSSSTATAVSHQWPKASRALPLIDLLHVFFNKTDLMHVTLGNMADKFGPIFSFPTGSHRTLVVSSWEKAKECFTGNNDIVFSGRPLPLAFKLIFYAGGIDSYGISQVPYGKKWRELRNICVHNILSNQQLLKFRHLMISQVDNSFNKLYEVCNSNKDEGDSATSTTAAGIVRMDDWLGKLAFDVIARIVCGFQSQTETSTTSSMERFTEAMDEASRFMSVTAVSDTVPWLGWIDQLTGLKRNMKHCGKKLNLVVKSIIEDHRQKRRLSSTKKGDENIIDEDEQDDFIDICLSIMEQPQLPGNNNPPKIPIKSIVLDMIGGGTDTTKLTTIWTLSLLLNNPHVLDKAKQEVDAHFLTKRRSTNDAAVVDFDDIRNLVYIQAIIKESMRLYPASPVVERLSGEDCVVGGFHVPAGTRLWVNVWKMQRDPNVWADPMVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 13 - protein, 858 aa, Papaver bracteatum
  REGION 1..858: note = partial-length amino acid sequence: SSDU-2015635
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSSKKLQELMATANIPPAVNQVEMSPAFQQKKLREYCNANNILVSAVSILGSNGTPWGSNAVLGSEVLKQIAMAKGKSVAQVSMRWVXKFSAYAIVWSLFFGHRICITLYSFLIRNVAYICITY

SEQ ID NO: 14 - protein, 716 aa, Papaver bracteatum
  REGION 1..716: note = partial-length amino acid sequence: SSDU-2015637
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRQVFLMQIRLIYICTYQQVHLNIYFQINEFVLCDMYRNLKLEY

SEQ ID NO: 15 - protein, 864 aa, Chelidonium majus
  REGION 1..864: note = partial-length amino acid sequence: chm.CMAST2PF_14984
LNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYPTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAFLKAIEVGYRYFDTAAAYETEEFLGEAIAEALQLGLIKSRDELFITSKLWPCDAHPDLVVPALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 16 - protein, 889 aa, Papaver bracteatum; identical to SEQ ID NO: 8
  REGION 1..889: note = DRS-DRR

SEQ ID NO: 17 - protein, 568 aa, Papaver bracteatum
  REGION 1..568: note = DRS
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERD

SEQ ID NO: 18 - protein, 321 aa, Papaver bracteatum
  REGION 1..321: note = DRR
MESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSCKKLQELMATANIPPAVNQVEMSPAFQQKKLREYCNANNILVSAVSILGSNGTPWGSNAVLGSEVLKQIAMAKGKSVAQVSMRWVYEQGASLVVKSFSEERLRENLNIFDWELTKEDNEKIGEIPQCRILTAYFLVSPNGPFKSQEELWDDKA

SEQ ID NO: 19 - protein, 153 aa, Papaver bracteatum
  REGION 1..153: note = Bet v1
MAPRGVSGLVGKLSTELDVNCDAEKYYNMYKNGEDVQKAVPHLCMDVKVISGDATRSGCIKEWNVNIDGKTIRSVEETTHNDETKTLRHRVFEGDMMKDYKKFDTIMEVNPKPDGNGCVVTRSIEYEKVNENSPTPFDYLQFGHQAMEDMNKY

SEQ ID NO: 20 - protein, 144 aa, Papaver setigerum
  REGION 1..144: note = Bet v1
MLVGKLSTELEVDCDAEKYYNMYKHGEDVKKALCVDVKVISGDPTRSGCIKEWNVNIDGKTIRSVEETTHNDETKTLRHRVFEGDMMKDFKKFDTIMVVNPKPDGNGCVVTRSIEYEKTNENSPTPFDYLQFGHQAIEDMNKYL

SEQ ID NO: 21 - protein, 141 aa, Papaver setigerum
  REGION 1..141: note = Bet v1
MLVGKLSTELEVDCDAEKYYNMYKHGEDKRQCVDVKVISGDPTRSGCIKEWNVNIDGKTIRSVEETTHNDETKTLRHRVFEGDMMKDFKKFDTIMVVNPKPDGNGCVVTRSIEYEKTNENSPTPFDYLQFGHQAIEDMNKY

SEQ ID NO: 22 - protein, 147 aa, Papaver setigerum
  REGION 1..147: note = Bet v1
MLVGKLSTELEVDCDAEKYYNMYKHGEDVKKAVPHLCVDVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYL

SEQ ID NO: 23 - protein, 109 aa, Papaver setigerum
  REGION 1..109: note = Bet v1
MVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYL

SEQ ID NO: 24 - protein, 180 aa, Papaver somniferum
  REGION 1..180: note = Bet v1
MDSINSSIYFCAYFRELIIKLLMAPPGVSGLVGKLSTELEVNCDAEKYYNMYKHGEDVQKAVPHLCVDVKVISGDPTRSGCIKEWNVNIDGKTIRSVEETTHNDETKTLRHRVFEGDVMKDFKKFDTIMVVNPKPDGNGCVVTRSIEYEKTNDNSPTPFDYLQFGHQAIEDMNKYLRDSE

SEQ ID NO: 25 - protein, 164 aa, Papaver somniferum
  REGION 1..164: note = Bet v1
MNFFIKDHLYICLVGKLSTELEVDCDAEKYYNMYKHGEDVKKAVPHLCVDVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYLRDSESN

SEQ ID NO: 26 - protein, 160 aa, Papaver somniferum
  REGION 1..160: note = Bet v1
MAPLGVSGLVGKLSTELEVDCDAEKYYNMYKHGEDVKKAVPHLCVDVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYLRDSESN

SEQ ID NO: 27 - protein, 400 aa, Papaver somniferum
  REGION 1..400: note = SalAT
MMKVCVSSREKIKPSRPTPGHLKTHKLSFLDQVAARIYVPLLLYYAGNKENVDTDTRCNIIKKSLAETLTKFYILAGKIVNDEIERFVNCNDDGVDFCVTKVSNCQLFQVIKRPDIFDQVTLFLPFDPCDNEITASGDFLLSVQVNVFEDCRGMVIGLCINHKVADASSITTFVNYWATIARGLVLNVDDRQIQDPCFQVQSIFPQKEKGIGFKISSSSIDGTLVTKKFGFEASKLAELKERCKFAGATEDIRGGYKPNRVEALSTFLWKCFIDIDQAKTKAAAPARVYLASNAVNIRSRIVPQLPTSSFGNMVAITDAIFTVNSNENNGINDPYYPKLVQKFRDAVKRVDGEYIEALQSTDLLLNNVTKLFKHILNGQTLSISFTSWCRFPFYDTDLLD

SEQ ID NO: 28 - protein, 386 aa, Papaver somniferum
  REGION 1..386: note = SalAT
MKVQVISKELIKPSTPTPPRLRNFKLSLLDQLLPPFYVPIIIFYPANDDHESNNNDQCIKANILKKSLSETLTRFYPIAGRIRDKILVECNDEGVHYIEAKVNAVMSDFMSLDVIHQLHPSYITLDDLAEEAQLAVQVTMFDCGGIALSICSSHKIIDGCTSTTFLNSWAATARAPSNPEIVYPTFDAAAIFPAQPSGVQVSTLESDDRLQGENVVTKRFLFSASKITALRARIAESRSSNILSKYPSRSEAVSALVWKSFMETSRVKVTREHTFSAEASTKPIVRSIANFVVNLRTRLNPPLPNVSFGNIIMDATAESLIIDNGENTLGFVETLDGLISQLRLGVTKMDDEYVRKLREDDVEFLKSLDEASHPSNGEGDGNGERV

SEQ ID NO: 29 - protein, 525 aa, Papaver setigerum
  REGION 1..525: note = SalAT
MNDTMKIEVVSKESIKPSYPTPNNLKIHNLSNLDQLIPAFYMDHILYYPSLDSNDSSLGDDEEDKKMIFSASSRHRCDVVKKSLAETLTRYYPLAGRIKDEKSVECNDEGVDYIEARVVGITVSQVIQLASSDIEVMEPFLPYEPYGGTGSAFRRAGIHSNSKPLLKIQVNVFDCGGMVICLSGSHKVIDATSILNFVNDWAATARGGFDTHDDELKVAVVDKPCYIFSSMFPPTSFGNQEEKDTADQAQLVPDRIEIVTKRFVFKDSSIAKLKKKCIHVNTNNGSDHQVDKQEHNMQQMPSRIEALTSLIWMCFMDVDRRFRVKQIDDAVSPVNTVNEVSLPKQVQYVAGFAINLRTRTIQPLPTNSFGNMTDTAIAEVTLNLTGSDHFNNEKGIRDQSQNYPELVSKIKDSIKLVDNKHIEAMKRNLAISCNNIKMHQMMKESTFDQNTRELLMFSSWCRFPIYEADFGWGKPSWASITKLLYKNCVMFLDTSSGDGIEAWVSLKEEDMVEFERHEELVALAS

SEQ ID NO: 30 - protein, 459 aa, Papaver somniferum
  REGION 1..459: note = SalAT
MKVQVISKEIIKPSSPTPPHLRNFKLSLLDQILPPFYVPIVMFYPAGDDYVTNNNIHDQSSKSEFLKKSLSETLTRFYPIAGRIKDNILIDCNNEGVDYIEAKVNGIMSDFMSVDVVHQLHPSHIMLDDVAKEAQLAVQVNLFDCGGIAISISMSHKIVDACTAITFINGWAATARAAPKQEIVCPTFDSAAIFPALPPGVQVSSLESDDSVQGVNVVTKMFAFTAPKIASLRARIAELRSSSDGLSKYPTRTEALSALVWKSFIRTSRVKAARKYSLSPASTKPVIKSVANYAVNLRTRLNPPLPQVSFGNILMDATAESTTTIDDDDSHEFADTLAGLIGQLRLGVSRINGDYIRKLQEGDLAFLKSLDEASHDSNGEKVQICWISSLCRFPFYEADFGWGKPSWVALNTNAEYKNSLFLMDTKCGTGIEAWVSLEEDDMAIFEEDQDLLQCVKSIN

SEQ ID NO: 31 - protein, 477 aa, Papaver setigerum
  REGION 1..477: note = SalAT
MENMKVEVVLKQTIKPSTQTPLHSKTFNLSFLDQHLGPPIYIPFTLYYESGDVNNKNNHCDGYKNNLEEACEHRVSVIKQSLSETLARYYPLAGRMKEDNLAVECNDEGVEYFETRVSDVRLSQVIKRSPNHNSVLRKFLPPCISSCDNSMSIPFDYGFKSKTLLAIQVNIFECGGIVIGMCMAHRLADASTMFTFITDWAATARGAIEDIKGPSFDFSYTLFPQKDVINNFKPFDPMLTREEDLVTKYFVFPASKIVELKRRNVNNIVCQDTSQQNTSPCTRVEAVTSFMWKRYMDSVRAKNQTQATSVEKYGALYTVNLRSRITPPLPANSFGNIYTFTIALSTPSDENDIDDGLRKDVSSPNDLNLVGKVRDAIKKIDDKYTRKLQSSEDELVNDVKPLTSGEAIFLGFSSWCRFPIYEADFGWGKPTWVSIGTMALRNTVFLMDTKSGDGIEAFVNMAKEDMDNFEVKLLADQ

SEQ ID NO: 32 - protein, 485 aa, Papaver setigerum
  REGION 1..485: note = SalAT
MENMKVEVVLEQTIKPSTQTPLHSKTFNLSFLDQHLGPPIYIPFTLYYESGDVNNKNNHCDGYKNNLEEVCEHRVSVIKQSLSETLARYYPLAGRMKEDNLAVECNDEGVEYFETRVSDVRLSQVIKRSPNHNSVLRKFLPPCISSCDNSMSIPFDYGFKSKTLLAIQVNIFECGGIVIGMCMAHRLADASTMFTFITDWAATARGAIEDIKGPSFDFSYTLFPQKDVINNFKPFDPMLTREEDLVTKYFVFPASKIVELKRRNVNNIVCQDTSQQNTSPCTRVEAVTSFMWKRYMDSVRAKNQTQATSVEKYGALYTVNLRSRITPPLPANSFGNIYTFTIALSTPSDENDIDDGLRKDVSSPNDLNLVGKVRDAIKKIDDKYTRKLQSSEDELVNDVKPLTSGEAIFLGFSSWCRFPIYEADFGWGKPTWVSIGTMALRNTVFLMDTKSGDGIEAFVNMAKEDMDNFEVKLLADQLLHVHPTV

SEQ ID NO: 33 - protein, 452 aa, Papaver setigerum
  REGION 1..452: note = SalAT
MSSTVEVISKQTIKPSTPTPIQRKNHSLSLIDQHFAPIYIPIVLFYPAAAVNDTGNVQHGDNTCVLKRSLSETLVHFYPLAGRMKDNIVVDCNDQGVEFTEVKVSGTMCDFLMKPDEQLSGLLPSEAVCMNFVREAQVMIQVNTFDCGSKAISLCVSHKIADASTITTFSRCWAETTIAVSKSTSAVTPIVSSKFHPTFDAASLFPPIKQLISPSGVTPALPELIPSEESKFGKIISKRFLFSATTINSVREKLSALMADKLKYRRLTRVEVVSALIWNSFDKLATTGSVAVMVKHAVNLRKRIDPPLPDVSFGNILEFTKAVVGEAAANTTTQGTVGSSSKLLEELSEFAGQLREPVSKMNKGDHDFDMENTDYEERDLWMSSWCNYGLYDIDFGCGKPVWVTTVATMYPYSDGFFMNDTRCGQGIEVWGNLVEEDMANFQLNLSELLDRI

SEQ ID NO: 34 - protein, 451 aa, Papaver somniferum
  REGION 1..451: note = SalAT
MMKVCVSSREKIKPSRPTPGHLKTHKLSFLDQVAARIYVPLLLYYAGNKENVDTDTRCNIIKKSLAETLTKFYILAGKIVNDEIERFVNCNDDGVDFCVTKVSNCQLFQVIKRPDIFDQVTLFLPFDPCDNEITASGDFLLSVQVNVFEDCRGMVIGLCINHKVADASSITTFVNYWATIARGLVLNVDDRQIQDPCFQVQSIFPQKEKGIGFKISSSSIDGTLVTKKFGFEASKLAELKERCKFTTEPEDGYKPTRVEALSAFLWKCFIDIDQAKLKGVARTKVYLATNAVNMRSRMVPQLPTSSFGNIISITDAVFSINNDDSTGINDPYYPKLVRKFRDAIKKIDRDYIEALRSTDLLLNNMMKLIEHVLSGHTLSIYFSSWCRFPLYETDFGWGKPIWVSTCTIPQKNVIVLMDSNSSADGIEAYVTLAKEDMGELEHHEELLALIS

SEQ ID NO: 35 - protein, 153 aa, Papaver bracteatum; identical to SEQ ID NO: 19
  REGION 1..153: note = THS

SEQ ID NO: 36 - protein, 144 aa, Papaver setigerum; identical to SEQ ID NO: 20
  REGION 1..144: note = THS

SEQ ID NO: 37 - protein, 141 aa, Papaver setigerum; identical to SEQ ID NO: 21
  REGION 1..141: note = THS

SEQ ID NO: 38 - protein, 147 aa, Papaver setigerum; identical to SEQ ID NO: 22
  REGION 1..147: note = THS

SEQ ID NO: 39 - protein, 109 aa, Papaver setigerum; identical to SEQ ID NO: 23
  REGION 1..109: note = THS

SEQ ID NO: 40 - protein, 180 aa, Papaver somniferum; identical to SEQ ID NO: 24
  REGION 1..180: note = THS

SEQ ID NO: 41 - protein, 164 aa, Papaver somniferum; identical to SEQ ID NO: 25
  REGION 1..164: note = THS

SEQ ID NO: 42 - protein, 160 aa, Papaver somniferum; identical to SEQ ID NO: 26
  REGION 1..160: note = THS

SEQ ID NO: 43 - protein, 158 aa, Papaver setigerum
  REGION 1..158: note = NPI
MAQNGDFGIVGKLVIELEVSSPADKFYTIFKHQKDVPKAIPHLFTDGKVIEGDARRSGCIKEWKYVLEGKTISVTEKTTHNDETKTLHHRIFEGDLMKDYKKFDSIIEVNPKPTGHGSIVTWSFVYEKINKNSPTPFAYLPFCYQAIEDINNHLAASE

SEQ ID NO: 44 - protein, 153 aa, Papaver setigerum
  REGION 1..153: note = NPI
MAHHGVSGLVGKLVTQLEVNCDADKLYKIYVPKAISHLFTGVKVLEGHGLRSGCIKEWKYIIDGKALTAVEETTHGDETRTLKHRVIDGDLMKDYKKFEKIIEANPKPNGHGSIVTVSLLYEKINEDSPAPFDHLKFFHQNIEDMNSHICASE

SEQ ID NO: 45 - protein, 152 aa, Papaver setigerum
  REGION 1..152: note = NPI
MARHSVSGLVGKLVTELEVSSDAEKYYKVYKHAEDVEKAIPHLCTGIRVIKGEASRSGCIKEWNFILEGKAIRSIEETTHNDATRTVHHRIFEGNLMKDYKKFDSIIEVNGCIVARSIVYEKRSEDSPTPFAYILFCHQAIEDMNKHLCDNE

SEQ ID NO: 46 - protein, 201 aa, Papaver setigerum
  REGION 1..201: note = NPI
MDSVSAALVFHSSIYLCAMAHHGVSGLVGKIVTELEVNCNADEFYKILKRDEDVPRAVSDLFPPVKIAKGDGLVSGCIKEWDCVLDGKAMSGKEETTHNDETRTLRHRELKKFDSIIEVNPKPNGHGSIVTWSIEYEKMNEDSPAPFAYLASFHQPNGHGSIVTWSIEYEKMNEDSPAPFAYLASFHQNVVEVDSHLCLSE

SEQ ID NO: 47 - protein, 184 aa, Papaver bracteatum
  REGION 1..184: note = NPI
MYSVSAALVSIAPYTFVRTDNNLRLLMACDGVSGLVGKLVTELKVNCDADKYYQIYKRPDDLQKAIPHLCTGIKLINGDASRSGCIKEWNFTLEGKRIHTVEETTHNDETRTLHHRIFEGDLMKDYKKFDSIIEVNPKPNGNGCVVKRSIVYEKINKDSPTPFSYLPFCHQAIEDMNKHLCDSE

SEQ ID NO: 48 - protein, 158 aa, Papaver bracteatum
  REGION 1..158: note = NPI
MACDGVSGLVGKLVTELKVNCDADKYYQIYKRPDDLQKAIPHLCTGIKLINGDASRSGCIKEWNFTLEGKRIHTVEETTHNDETRTLHHRIFEGDLMKDYKKFDSIIEVNPKPNGNGCVVKRSIVYEKINKDSPTPFSYLPFCHQAIEDMNKHLCDSE

SEQ ID NO: 49 - protein, 166 aa, Papaver bracteatum
  REGION 1..166: note = NPI
MAHHGVSGLVGKLVTQLEVNCDADEFYKIWKHHEEVPQAVSHLFPAVKVVKGDGLVSGCIKEWDYILEGKAMSAMEETTHNDETRTLHHRIVEGEVMKDYKAIASIIEVNPNPNGHGSIVTWSIEYEKMNEDSPTPFAYLEFFHQNLVDMNSHLYVGSDSHLHVDE

SEQ ID NO: 50 - protein, 158 aa, Papaver rhoeas
  REGION 1..158: note = NPI
MAPHGVSDLSGKLVTELEVSCDADKYYKIYKHAEDVQKAVPHLCTDVKVINGDATLSGCIKEWHYILEGKALSAKEETTINDETRTLHHRVLEGDMMKDYKKFDSVIEVNPKPNGNGSVVTRSIAYEKINEDSPTPFAYILFSHRAVEDMNKYLCDSE

SEQ ID NO: 51 - protein, 157 aa, Papaver rhoeas
  REGION 1..157: note = NPI
MAPHGVSDLSGKLVTELEVSCDADKYYKIYKHAEDVQKAVPHLCTDVKVINGDATLSGCIKEWHYILEGKALSAKEETTINDETRTLHHRVLEGDMMKDYKKFDSVIEVNPKPNGNGSVVTRSIAYEKINEDPAPFAYLAFFHQNAVEVNSYLCLSE

SEQ ID NO: 52 - protein, 147 aa, Papaver rhoeas
  REGION 1..147: note = NPI
MAPHGVSDLSGKLVTELEVSCDADKYYKIYKHAEDVQKAVPHLCTDVKVINGDATLSGCIKEWHYILEGKALSAKEETTINDETRTLHHRVLEGDMMKDYKKFDSVIEVNPKPNGNGSVVTRSIAYEKINEDSSSLCVSSFLPSERG

SEQ ID NO: 53 - protein, 159 aa, Papaver rhoeas
  REGION 1..159: note = NPI
MAHHGVSGLVGKLVTQLEVNCDADKFYKMAKHHEDVPKAVPHFFTAVKVTEGDGLVSGCIKEWDYILEGKAMSCKEEEETTHYDETRTLHHRVFGGDMMMDYKKFDAIIEVNPKPNVHGCIVTWSIAYEKINEDSPVPFDYLAFYHQNIIDVGSHLCSE

SEQ ID NO: 54 - protein, 176 aa, Papaver somniferum
  REGION 1..176: note = NPI
MDSVSAALVFHSSIYLCAMAHHGVSGLVGKIVTELEVNCNADEFYKILKRDEDVPRAVSDLFPPVKIAKGDGLVSGCIKEWDCVLDGKAMSGKEETTHNDETRTLRHRELEGDLMKDYKKFDSIIEVNPKPNGHGSIVTWSIEYEKMNEDSPAPFAYLASFHQNVVEVDSHLCLSE

SEQ ID NO: 55 - protein, 158 aa, Papaver somniferum
  REGION 1..158: note = NPI
MAHHGVSGLVGKIVTELEVNCNADEFYKILKRDEDVPRAVSDLFPPVKIAKGDGLVSGCIKEWDCVLDGKAMSGKEETTHNDETRTLRHRELEGDLMKDYKKFDSIIEVNPKPNGHGSIVTWSIEYEKMNEDSPAPFAYLASFHQNVVEVDSHLCLSE

SEQ ID NO: 56 - protein, 162 aa, Papaver somniferum
  REGION 1..162: note = NPI
MAHHGISGLVGKLVTQLEVNCDADEFYKIWKHHEEVPKAVSHLLPAVKVVKGDGLVSGCIKEWHYILEGKAMSAMEETTHNDETRTLHHQVVEGELMKDYKAIASIIQVNPNGSIVTWSIEYEKMNEDSPTPFAYLEFFHQNIIDMNSHLYVGSDSHLHVDE

SEQ ID NO: 57 - protein, 157 aa, Papaver somniferum
  REGION 1..157: note = NPI
MAHHGVSGLVGKLVTELEVHCNADAYYKIFKHQEDVPKAMPHLYTGGKVISGDATRSGCIKEWNYILEGKALIAVEETTHDDETRTLTHRITGGDLTKDYKKFVKIVEVNPKPNGHGSIVTVSLVYEKMNEGSPTPFNYLQFVHQTIVGLNSHICAS

SEQ ID NO: 58 - protein, 158 aa, Papaver somniferum
  REGION 1..158: note = NPI
MAHHGISGLVGKLVIGLEVNCDADKYYQIFKHAEDVQKAVPHHYDSIKVINGDAKSSGCIKEWNFIHEGKTFHTVEETTHNDETRTLHHRIFEGDLMKDYKKFDLIIEANPKPTGNGCVVTWTIEYEKINQDSPAPIAYLPFCNQVIEDMNKHLCDSE

SEQ ID NO: 59 - protein, 321 aa, Papaver somniferum
  REGION 1..321: note = COR 1.3
MESNGVPMITLSSGIRMPALGMGTAETMVKGTEREKLAFLKAIEVGYRHFDTAAAYQSEECLGEAIAEALQLGLIKSRDELFITSKLWCADAHADLVLPALQNSLRNLKLDYLDLYLIHHPVSLKPGKFVNEIPKDHILPMDYKSVWAAMEECQTLGFTRAIGVCNFSCKKLQELMAAAKIPPVVNQVEMSPTLHQKNLREYCKANNIMITAHSVLGAICAPWGSNAVMDSKVLHQIAVARGKSVAQVSMRWVYQQGASLVVKSFNEGRMKENLKIFDWELTAENMEKISEIPQSRTSSADFLLSPTGPFKTEEEFWDEKD

SEQ ID NO: 60 - protein, 321 aa, Papaver somniferum
  REGION 1..321: note = COR
MESNGVPMITLSSGIRMPALGMGTVETMEKGTEREKLAFLKAIEVGYRRFDTAAAYQTEECLGEAIAEALQLGLIKSRDELFITSKLWCADAHADLVLPALQNSLRNLKLDYLDLYLIHHPVSLKPGKFVNEIPKDHILPMDYKSVWAAMEECQTLGFTRAIGVCNFSCKKLQELMATANSPPVVNQVEMSPTLHQKNLREYCKANNIMITAHSVLGAIGAPWGSNAVMDSKVLHQIAVARGKSVAQVSMRWVYQQGASLVVKSFNEARMKENLKIFDWELTAEDMEKISEIPQSRTSSAAFLLSPTGPFKTEEEFWDEKD

SEQ ID NO: 61 - protein, 320 aa, Papaver somniferum
  REGION 1..320: note = COR
MESNGVPMITLSSGIRMPALGMGTAETMVKGTEREKLAFLKAIEVGYRHFDTAAAYQSEECLGEAIAEALQLGLIKSRDELFITSKLWCADAHADLVLPALQNSLRNLKLDYLDLYLIHHPVSLKPGKFVNEIPKDHILPMDYKSVWAAMEECQTLGFTRAIGVCNFSCKKLQELMATANSPPVVNQVEMSPTLHQKNLREYCKANNIMITAHSVLGAVGAAWGTNAVMHSKVLHQIAVARKSVAQVSMRWVYQQGASLVVKSFNEARMKEDLKIFDWELTAEDMEKISEIPQSRTSSAAFLLSPTGPFKTEEEFWDEKD

SEQ ID NO: 62 - protein, 321 aa, Papaver somniferum
  REGION 1..321: note = COR
MESNGVPMITLSSGIRMPALGMGTVETMEKGTEREKLAFLKAIEVGYRHFDTAAAYQTEECLGEAIAEALQLGLIKSRDELFITSKLWCADAHADLVLPALQNSLRNLKLDYLDLYLIHHPVSLKPGKFVNEIPKDHILPMDYKSVWAAMEECQTLGFTRAIGVCNFSCKKLQELMATANSPPVVNRVEMSPTLHQKNLREYCKANNIMITAHSVLGAVGAAWGTNAVMHSKVLHQIAVARGKSVAQVSMRWVYQQGASLVVKSFNEARMKENLKIFDWELTAEDMEKISEIPQSRTSSAAFLLSPTGPFKTEEEFWDEKD

SEQ ID NO: 63 - protein, 321 aa, Papaver somniferum
  REGION 1..321: note = COR
MESNGVPMITLSSGIRMPALGMGTAETMVKGTEREKLAFLKAIEVGYRHFDTAAAYQSEECLGEAIAEALQLGLIKSRDELFITSKLWCADAHADLVLPALQNSLRNLKLEYLDLYLIHHPVSLKPGKFVNEIPKDHILPMDYKSVWAAMEECQTLGFTRAIGVCNFSCKKLQELMATANSPPVVNQVEMSPTLHQKNLREYCKANNIMITAHSVLGAVGAAWGTNAVMHSKVLHQIAVARGKSVAQVSMRWVYQQGASLVVKSFNEARMKENLKIFDWELTAEDVEKISEIPQSRTSSAAFLLSPTGPFKTEEEFWDEKD

SEQ ID NO: 64 - protein, 321 aa, Papaver somniferum
  REGION 1..321: note = COR
MESNGVPMITLSSGIRMPALGMGTVETMEKGTEREKLAFLKAIEVGYRHFDTAAAYQTEECLGEAIAEALQLGLIKSRDELFITSKLWCADAHADLVLPALQNSLRNLKLDYLDLYLIHHPVSLKPGKFVNEIPKDHILPMDYKSVWAAMEECQTLGFTRAIGVCNFSCKKLQELMATANSPPVVNQVEMSPTLHQKNLREYCKANNIMITAHSVLGAVGAAWGTNAVMHSKVLHQIAVARGKSVAQVSMRWVYQQGASLVVKSFNEARMKENLKIFDWELTAEDMEKISEIPQSRTSSADFLLSPTGPFKTEEEFWDEKD

SEQ ID NO: 65 - protein, 321 aa, Papaver setigerum
  REGION 1..321: note = COR
MESNGVPMITLSSGIRMPALGMGTVETMEKGTEREKLAFLKAIEVGYRHFDTAAAYQTEECLGEAIAEALQLGLIKSRDELFITSKLWCADAHADLVLPALQNSLRNLKLDYLDLYLIRHPVSLKPGKFVNEIPKDHILPMDYKSVWAAMEECQTLGFTRAIGVCNFSCKKLQELMATVNSPPVVNQVEMSPTLHQKNLREYCKANNIMITAHSVLGAVGAAWGTKAVMHSKVLHQIAVARGKSVAQVSMRWVYQQGASLVVKSFNEARMKENLKIFDWELTAEDMEKISEIPQSRTSSAAFLLSPTGPFKTEEEFWDEKD

SEQ ID NO: 66 - protein, 321 aa, Papaver bracteatum
  REGION 1..321: note = COR
MESNGVPMITLSSGIRMPALGMGTVETMEKGTEREKLAFLKAIEVGYRHFDTAAAYQTEECLGEAIAEALQLGLIKSREELFITSKLWCTDAHADLVLPALQNSLRNLKLEYLDLYLIHFPVSLKPGKIVSDIPKDQMLPMDYKSVWVAMEECQTLGFTRAIGVSNFSCKKLQELMATANSPPVVNEVEMSPVFQQKNLRAYCKANNIMITAYSVLGARGAAWGSNAVMDSKVLHEIAVARGKSVAQVSMRWVYQQGACLVVKSFNEERMKENLKIFDWELSAEDMEMISEIPQCRTSSADFLLSPTGPFKTEEEFWDEKD

SEQ ID NO: 67 - protein, 321 aa, Papaver somniferum
  REGION 1..321: note = COR 1.3 Mutant
MESNGVPMITLSSGIRMPALGMGTAETMVKGAEREKLAFLKAIEVGYRHFDTAAAYQSEECLGEAIAEALQLGLIKSRDELFITSKLWCADAHADLVLPALQNSLRNLKLDYLDLYLIHHPVSLKPGKFVNEIPKDHILPMDYKSVWAAMEECQTLGFTRAIGVCNFSCKKLQELMAAAKIPPVVNQVEMSPTLHQKNLREYCKANNIMITAHSVLGAICAPWGSNAVMDSKVLHQIAVARGKSVAQVSMRWVYQQGASLVVKSFNEGRMKENLKIFDWELTAENMEKISEIPQSRTSSADFLLSPTGPFKTEEEFWDEKD

SEQ ID NO: 68 - protein, 321 aa, Papaver somniferum
  REGION 1..321: note = COR 1.3 Mutant
MESNGVPMITLSSGIRMPALGMGTAETMVKGTEREKLAFLKAIEVGYRHFDTAAAYQSEECLGEAIAEALQLGLIKSRDELFITSKLWCADAHADLVLPALQNSLRNLKLDYLDLYLIHHPVSLKPGKFVNEIPKDHILPMDYKSVWAAMEECQTLGFTRAIGVCNFSCKKLQELMAAAKIPPVVNQVEMSPTLHQKNLREYCKANNIMITAHSVLGAICAPWGSNAVMDFKVLHQIAVARGKSVAQVSMRWVYQQGASLVVKSFNEGRMKENLKIFDWELTAENMEKISEIPQSRTSSADFLLSPTGPFKTEEEFWDEKD

SEQ ID NO: 69 - protein, 196 aa, Coptis japonica
  REGION 1..196: note = CjNCS full length
MRMEVVLVVFLMFIGTINCERLIFNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLAIMSEAIAKVVLENKHKSSE

SEQ ID NO: 70 - protein, 173 aa, Camellia japonica
  REGION 1..173: note = NCS parent
MNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLAIMSEAIAKVVLENKHKSSE

SEQ ID NO: 71 - protein, 173 aa, Camellia japonica
  REGION 1..173: note = NCS1
MNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLANMSEAIAKVVLENKHKSSE

SEQ ID NO: 72 - protein, 173 aa, Camellia japonica
  REGION 1..173: note = NCS2
MNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLITTVPLANMSEAIAKVVLENKHKSSE

SEQ ID NO: 73 - protein, 173 aa, Camellia japonica
  REGION 1..173: note = NCS3
MNGRPLLHRVTKEETVMLYHELEVAASADEVWSVEGSPELGLHLPDLLPAGIFAKFEITGDGGEGSILDITFPPGQFPHHYREKFVFFDHKNRYKLVEQIDGDFFDLGVTYYMDTIRVVATGPDSCVIKSTTEYHVKPEFAKIVKPLITTVPLANMSEAIAKVVLENKHKSSE

SEQ ID NO: 74 - protein, 210 aa, Thalictrum flavum
  REGION 1..210: note = TfNCS PDB: 5N8Q
MMKMEVVFVFLMLLGTINCQKLILTGRPFLHHQGIINQVSTVTKVIHHELEVAASADDIWTVYSWPGLAKHLPDLLPGAFEKLEIIGDGGVGTILDMTFVPGEFPHEYKEKFILVDNEHRLKKVQMIEGGYLDLGVTYYMDTIHVVPTGKDSCVIKSSTEYHVKPEFVKIVEPLITTGPLAAMADAISKLVLEHKSKSNSDEIEAAIITV

SEQ ID NO: 75 - protein, 210 aa, Thalictrum flavum; identical to SEQ ID NO: 74
  REGION 1..210: note = TfNCS

SEQ ID NO: 76 - protein, 220 aa, Argemone mexicana
  REGION 1..220: note = AmNCS
MSKLITTAPLKSMSEAIANYVLKKQSVIRKVVTYELEVPASADSIWAVYSSPNIPTLLRDVLLPGVFEKLDVIEGNGGVGTVLDIVFPPGAVPRRYKEKFVKINNEKRLKEVIMIEGGYLDMGCTSYMDRIHVLEKTPNSCVIESSIIYEVKQEYADEMSKLITTVPLKSMSEVIANYVLKKQFRVFGYEIKPKLGLSLLLCLIICLVILGGLLIAGVPV

SEQ ID NO: 77 - protein, 194 aa, Podophyllum hexandrum
  REGION 1..194: note = ShNCS
MRMEIVSLFLFFLACGTSQGRPLLESKLYRKSTIQKVLHHESTVAASAEEVWQVYSSPELPKHLSELLPGAFEKVEIFGDGGVGTILEMVFPPGEIPQSYKEKFVLIDDERRLKKVEMIEGGYLDMGVTFYMDTIHIVATGCNSCIIKSSTEYYVKPEFADKVQPLITTAPLEAMDEAITKIVLANKNKSLIEI

SEQ ID NO: 78 - protein, 193 aa, Papaver bracteatum
  REGION 1..193: note = PbNCS
MMRKVIKYDMEVATSADSVWAVYSSPDIPRLLRDVLLPGVFEKLDVIEGNGGVGTVLDIAFPPGAVPRSYKEKFVNIDRVKRLKEVIMIEGGYLDMGCTFYLDRIHVVEKTPNSCVIESSIIYEVKEEFADKMAKLITTEPLQSMAEVISGYVLKKRLQVFGFEIKPNLRFNLLLCLIICLVIAGGMLIGRVP

SEQ ID NO: 79 - protein, 231 aa, Papaver somniferum
  REGION 1..231: note = PsNCS2
MSKLITTEPLKSMAEVISNYVIQRESFSARNILNKNSLVKKEIRYDLEVPTSADSIWSVYSCPDIPRLLRDVLLPGVFQKLDVIEGNGGVGTVLDIVFPPGAVPRSYKEKFVNINHEKRLKEVIMIEGGYLDMGCTFYMDRIHIFEKTPNSCVIESSIIYEVKEEYAGKMAKLITTEPLESMAEVISGYVLKKRLQVFGFEIKPKLRFNLLLCLIICLVIAGGMFVAGVPL

SEQ ID NO: 80 - protein, 200 aa, Papaver somniferum
  REGION 1..200: note = PsNCS3
MRKVIKYDMEVAVSADSVWAVYSSPDIPRLLRDVLLPGVFEKLDVIEGNGGVGTVLDIVFPPGAVPRSYKEKFVNIDREKRLKEVIMIEGGYLDMGCTFYLDRIHVVEKTKSSCVIESSIVYDVKEECADAMSKLITTEPLKSMAEVISNYVIQKELFSARNILSKQSVVKKEIRYDLEVPISVDSIWSVYSCPDIPRLL

SEQ ID NO: 81 - protein, 206 aa, Corydalis saxicola
  REGION 1..206: note = CsNCS
MGKRIQKKEAKKILRKELTHELEVPTSADSIWAVYGSPDIPRLLRDVLLPGVFEKLDIIEGNGGVGTVLDIAFPPGTVPRSYKEKFVKVDHDKHLKEVVMIEGGYLDLGCTFYMDRIHVLPKGPNSCVIESSLIYEVREELADAVGSLISIEPLASMAEVVSSYVLKQQLRVFGVVVQPRVGLSLLLCLILCLVILGGLLIGGVSI

SEQ ID NO: 82 - protein, 188 aa, Papaver somniferum
  REGION 1..188: note = PsPR10-1
MRYELINEFDVGASADDVWAVYSSPDLPRLIVELLPGVFKKIDVVEGNGGLGTVLHLVYPHGSVPLSYKEKFVTMDNHKRLKEVRQIEGGYLEMGCTFYMDSFQILKKTHDSCTIRSITKYEVSAELAEKVSSHISVESLVDMARAISKYVIQDNEKNKAEDPSTCSEEGHDKKHGHLHRIWISLFGK

SEQ ID NO: 83 - protein, 364 aa, Papaver somniferum
  REGION 1..364: note = T6ODM
MEKAKLMKLGNGMEIPSVQELAKLTLAEIPSRYVCANENLLLPMGASVINDHETIPVIDIENLLSPEPIIGKLELDRLHFACKEWGFFQVVNHGVDASLVDSVKSEIQGFFNLSMDEKTKYEQEDGDVEGFGQGFIESEDQTLDWADIFMMFTLPLHLRKPHLFSKLPVPLRETIESYSSEMKKLSMVLFNKMEKALQVQAAEIKGMSEVFIDGTQAMRMNYYPPCPQPNLAIGLTSHSDFGGLTILLQINEVEGLQIKREGTWISVKPLPNAFVVNVGDILEIMTNGIYHSVDHRAVVNSTNERLSIATFHDPSLESVIGPISSLITPETPALFKSGSTYGDLVEECKTRKLDGKSFLDSMRI

SEQ ID NO: 84 - protein, 360 aa, Papaver somniferum
  REGION 1..360: note = CODM
METPILIKLGNGLSIPSVQELAKLTLAEIPSRYTCTGESPLNNIGASVTDDETVPVIDLQNLLSPEPVVGKLELDKLHSACKEWGFFQLVNHGVDALLMDNIKSEIKGFFNLPMNEKTKYGQQDGDFEGFGQPYIESEDQRLDWTEVFSMLSLPLHLRKPHLFPELPLPFRETLESYLSKMKKLSTVVFEMLEKSLQLVEIKGMTDLFEDGLQTMRMNYYPPCPRPELVLGLTSHSDFSGLTILLQLNEVEGLQIRKEERWISIKPLPDAFIVNVGDILEIMTNGIYRSVEHRAVVNSTKERLSIATFHDSKLESEIGPISSLVTPETPALFKRGRYEDILKENLSRKLDGKSFLDYMRM

SEQ ID NO: 85 - protein, 364 aa, Papaver somniferum
  REGION 1..364: note = PsP7ODM
MEKAKLMKLGNGLSIPSVQELAELTFAEVPSRYVCTNDENLLLMTMGASEIDDETVPVIDLQNLLSPEPAIGKSELDWLHYSCKEWGFFQLVNHGVDALLVDHVKSEIHSFFNLPLNEKTKYGQRDGDVEGFGQAFLVSENQKLDWADMFFINTLPLHLRKPHLFPNLPLPLRETIESYSSEMKKLSMVLFEMMGKAIEVIDIKEAITEMFEDGMQSMRMNYYPPCPQPERVIGITPHSDFDGLTILLQLNEVEGLQIRKEDKWISIKPLPDAFIVNVGDIWEIMTNGVHRSVDHRGVINSTKERLSIATFHSPKLELEIGPISSLIRPETPAVFKSAGRFEDLLKEGLSRKLDGKSFLDCMRM

SEQ ID NO: 86 - protein, 294 aa, Papaver somniferum
  REGION 1..294: note = PsoDIOX1
MEKAKLMKLGNGMEIPSVQELAKLTLAEIPSRYVCANENLLLPMGASVINDHETIPVIDIENLLSPEPIIGKLELDRLHFACKEWGFFQVVNHGVDASLVDSVKSEIQGFFNLSMDEKTKYEQEDGDVEGFGQGFIESEDQTLDWADIFMMFTLPLHLRKPHLFSKLPVPLRETIESYSSEMKKLSMVLFNKMEKALQVQAAEIKGMSEVFIDGTQAMRMNYYPPCPQPNLAIGLTSHSDFGGLTILLQINEVEGLQIKREGTWISVKPLPNAFVVNVGDILEIMTNGIYHSVD

SEQ ID NO: 87 - protein, 364 aa, Papaver somniferum
  REGION 1..364: note = PsoDIOX2
METAKLMKLGNGMSIPSVQELAKLTLAEIPSRYICTVENLQLPVGASVIDDHETVPVIDIENLISSEPVTEKLELDRLHSACKEWGFFQVVNHGVDTSLVDNVKSDIQGFFNLSMNEKIKYGQKDGDVEGFGQAFVASEDQTLDWADIFMILTLPLHLRKPHLFSKLPLPLRETIESYSSEMKKLSMVLFEKMEKALQVQAVEIKEISEVFKDMTQVMRMNYYPPCPQPELAIGLTPHSDFGGLTILLQLNEVEGLQIKNEGRWISVKPLPNAFVVNVGDVLEIMTNGMYRSVDHRAVVNSTKERLSIATFHDPNLESEIGPISSLITPNTPALFRSGSTYGELVEEFHSRKLDGKSFLDSMRM

SEQ ID NO: 88 - protein, 369 aa, Papaver bracteatum
  REGION 1..369: note = PbrDIOX2
METPKSIKLGGSLLVPSVQELAQQSFAEVPARYVRDDLEPLTDLSGVSMIDQTIPVIDLQKLQSPVPIIRELESEKLHSACKEWGFFQVVNHGVDILLVEKTKSEIKDFFNLPMDEKKKFWQEEGDIQGFGQAFVQSEDQKLDWADIFLMVTLPRHTRNPRLFPKLPLPLRNTMDSYSSKLSKLASTLIEMMGKALHMETSVLAELFEDGRQTMRINYYPPCPQPKDVIGLTPHSDGGGLTILLQLNEVDGLQIRKEKIWIPIKPLPNAFVVNIGNILEIMTNGIYRSVEHRATIHSTKERLSVAAFHNPKVGVEIGPIVSMITPESPALFRTIEYDDYGKKYFSRKLDGKSSLDFMRIGEGDEENKAT

SEQ ID NO: 89 - protein, 366 aa, Papaver bracteatum
  REGION 1..366: note = PbrDIOX3
METPKLIKLGGSLLVPSVLELTKQSPAEVPARYIRNDLEPMTDLSSASLTDQTIPVIDLQNLLSPEPELELEKLHSGCKEWGFFQVMNHGVDILLVEKVKSEIQGFFNLPIDEKNKFWQEEGDLEGYGKAFVHSEDEKLDWADMFFILTQPQYMRKPRVFPKLPLRLRETIESYSLELSKLGLTLLDLMGKALQIETGVMSELFEDGRQTMRMNYYPPCPQPEHVIGLTPHSDGGALTILLQLNQVDGLQIRKEEIWVPIKPLPNAFVVNIGDILEIMSNGVYRSVEHRATINSSKERLSVAIFQSPKHGTEIGPILSMITPEAPALFKTIPYEDYLRKFFSRKLGGKSFVDSMRIGESDEDNNTA

SEQ ID NO: 90 - protein, 366 aa, Papaver bracteatum
  REGION 1..366: note = PbrDIOX4
METQKQENFGASLSVPNVQELAKQSPEQVPDRYIRSDQDSSTNISCPSMTDQIPVIDLQSLLSPDPIIGELELERLHSACKEWGFFQVVNHGVDNLLVEKVKSEIQGFFNLPMDEKKKFWQEEGDFEGFGQAFVFSEDQKLDWGDVFFILTQPQHMRKPRLFPKLPLPFRKTIESYSLETNKLSMTLLELMEKALKIETGVMTELFEGGIQRMRMTYYPPCPQPKHVIGLTPHSDPDALTILLQLNEVDGLQIRKEKIWVPIKPLSNAFVVNIGDILEIMSNGIYRSVEHRATVNSTKERLSVATFHSPRKDTEIGPILITPETPALFRTSGFEDYFRKFFAHKLNGKSFLSSIRIGETDEGNNAT

SEQ ID NO: 91 - protein, 379 aa, Papaver bracteatum
  REGION 1..379: note = PbrDIOX5
MEAPKLIMLGGSLFVPSVQELAKQSLAEVPVRYVRDDQDTLGNNINITPMSMIDQSIPVIDLEKLLSPEPIVGELELERLHSACKEWGFFQVVNHGVDSLLVEKVKSEIEGFFKLPMDEKTKFWQEEGDIEGFGQVFVHSQDQKLDWGDMFLMQTLPRHTRKPRLFPNLPLPLRQTIESYSSELSKLVLTLVDLMGKALQMESGVLTELFENGIQRMRMNYYPPCPQPEQVIGLTPHSDVGGLTILLQLNEVDGLQIKKDKVWVPIKPLANAFVVNVGDALEIMSNGIYRSVEHRATINSTKERLSIATFHNPRADREIGPIPSMISPETPALFKTTGYEEYFKKFFSRKLEGKSFLDSLRIREGDEHCGRLDVKGPCN

SEQ ID NO: 92 - protein, 365 aa, Papaver bracteatum
  REGION 1..365: note = PbrDIOX6
MEIPNPIKIGSSLLVPSVQELAKQSFAEVPARYIRNDVDPLITKLSDVSLIDQTVPVIDLQKLLSPEPIVGELELERLHSACKEWGFFQVVNHGVDNLLVEKVKSEIQGFFNLPMEEKKKFWQEEGDFEGFGQMFVQSEEQKLDWGDMFFILTQPQHMRKPRLFSKLPLPLRETIESYSLELIKLGLTIIKLMEKALQIDAGVMAELFEDGIHTMRMNYYPPCPQPEHVIGLTPHSDGGGLTILLQLNEVDGLQIRRENIWVPIKPLPNAFVVNIGDILEILSNGIYRSVEHRSTVNATKERLSVATFQNPKQESVIGPNMITPERPALFRKIVYKDYMKKLFSRKLDGKSFLDSLRIGEGDERP

SEQ ID NO: 93 - protein, 349 aa, Papaver bracteatum
  REGION 1..349: note = PbrDIOX8
METLKTVKPGGSLFIPNGQELAKQSLEEVYVGNDQDTMLLIGQTIPVIDLQKLLSPEPITGDMELDKLHSACKEWGFFQVVNHGVDILLVEKVKSEVHDFFNIPMDEKKPFWQEEGDLEGFGQVFITSEDQQLDWGDMFFMVTLPKHMRKPRLFLKLPLPLRETIESYSLKLSKLGVTLVELMGKALQMEDRIMSELFDDGRQTMRMNYYPPCPQPEQVIGLTPHSDPGGLTILLELNEVNGLIRKENIWVPIIPLPNAFIVNIGDILEIMSNGIYHSVEHRATINSTKERLSVAMFNSPKVDTEIGPIHSMITPETPALFRTIGYDEYLKIFFSRKLDGKSLLESMKI

SEQ ID NO: 94 - protein, 379 aa, Papaver bracteatum
  REGION 1..379: note = PbrDIOX10
MEAPKLIMLGGSLFVPSVQELAKQSLAEVPVRYVRDDQDTLGNNINITPMSMIDQSIPVIDLEKLLSPEPIVGELELERLHSACKEWGFFQVVNHGVDSLLVEKVKSEIEGFFELPVDEKKKFWQEEGDIEGFGQIFVHSEDQKLDWADMFYMLTLPPNMRKPRLFPNLPLPLRQTIDSYSSELSKLVLTLVDLMGKALQMESGVLTELFENGIQRMRMNYYPPCPQPEQVIGLTPHSDVGGLTILLQLNEVDGLQIKKDKIWVPIKPLRNAFVVNVGDALEIMSNGIYRSVEHRATINSTKERLSIATFHNPRADREIGPIPSMISPETPALFKTTGYEEYFKKFFSRKLEGKSFLDSLRIGEGDEHCGRLXVKGXCN

SEQ ID NO: 95 - protein, 362 aa, Papaver bracteatum
  REGION 1..362: note = PbrDIOX11
METPKLMKLGGSLFVPSVQELAKQSLAEVPARYVRDDRDMVGNIINVTPMSMIDQSIPVIDLEKLLSPDLIVGELELERLHSACKEWGFFQVVNHGVDSLLVEKVKSEIEGFFELPMDEKKKFWQEEGDAEGFAQFFVQSEDQKLDYSGDMFFMLNLPQHMRKPRLFLKLPLPLRETIESYSLKLSKLGVTLVELMGKALQMEDRIMSELFDDGRQTMRMNYYPPCPQPEQVIGLTPHSDPGGLTILLELNEVNGLIRKENIWVPIIPLPNAFIVNIGDILEIMSNGIYHSVEHRATINSTKERLSVAMFNSPKVDTEIGPIHSMITPETPALFRTIGYDEYLKIFFSRKLDGKSLLESMKI

SEQ ID NO: 96 - protein, 365 aa, Papaver bracteatum
  REGION 1..365: note = PbrDIOX13
METPKLRDFGSFLPVPSVQELAKQVLTEIPPRYIRTDLEALNKLSCASNTDQTVPIIDMQCLLSAEPEMELEKLHSACKEWGFFRVVNHGVDNLESVKSEIESFLNLPVNAKNKYGQKQGDDQGFGSRFVLSEEQKLDWGDFFYMVTRPLYLRKPHLFPELPLPLRETIESYSSEVSKLAMALFEMMGKALKIETGVMTEIFEGGMQAMRMNYYPPCPRPDLVIGLNAHSDFGGLTILLQLNEVEGLEIRNKGEWVSVKPLANAFVVNVGDVMEILTNGIYHSVEHRATINSSKERLSVATFHYPKLETGIGPLPCMITPKTPALFGRIERYELLLRKYYARKLNGKSTLDCMRIGNGFEDDNTA

SEQ ID NO: 97 - protein, 370 aa, Papaver bracteatum
  REGION 1..370: note = PbrDIOX18
MEAPKLIMLGGSLFVPSVQELAKQSLAEVPARYVRDDQDTLGNNINITPMSMIDQSIPVIDLEKLLSPEPIVGELELERLHSACKEWGFFQVVNHGVDSLLVEKVKSEIEGFFELPVDEKKKFWQEEGDIEGFGQIFVHSEDQKLDWADMFYMLTLPPNMRKPRLFPNLPLPLRQTIDSYSSELSKLVLTLVDLMGKALQMESGVLTELFENGIQRMRMNYYPPCPQPEQVIGLTPHSEVGGLTILLQLNEVDGLQIRKEKIWVPIKPLSNAFIVNIGDILEIMSNGIYRSVEHRATVNSTKERLSVATFHSPRKDTEIGPILITPETPALFRTSGFEDYFRKFFAHKLNGKSFLSSIRIGETDEGNNAT

SEQ ID NO: 98 - protein, 313 aa, Papaver bracteatum
  REGION 1..313: note = PbrDIOX19
MSMIDQSIPVIDLEKLLSPEPIVGELELERLHSACKEWGFFQVVNHGVDSLLVEKVKSEIEGFFELPVDEKKKFWQEEGDIEGFGQIFVHSEDQKLDWADMFYMLTLPPNMRKPRLFPNLPLPLRQTIDSYSSELSKLVLTLVDLMGKALQMESGVLTELFENGIQRMRMNYYPPCPQPEQVIGLTPHSDVGGLTILLQLNEVDGLQIRKEKIWVPIKPLSNAFIVNIGDILEIMSNGIYHSVEHRATINSTKERLSVAMFNSPKVDTEIGPIHSMITPETPALFRTIGYDEYLKIFFSRKLDGKSLLESMKI

SEQ ID NO: 99 - protein, 365 aa, Papaver bracteatum
  REGION 1..365: note = PbrDIOX21
METPKLVKSSGSSLFLSTSVQELAKQSLPEVPARYIRTNLEPLSNVSGDSQSVPVIDLQKLLSSEPIIGELELDKLHSACKEWGFFQVVNHGVDNLVMEKIKTEIQGFFNLSLDEKQKFWKKEGDAEGFGQNFIESEDQKLDWGDTFGMFTLPIHMRNPRLFPELPLPLRETIESYSLDVRKLALALIGLMEKALKIKTSAMSELFEDGGQAMRMNYYPPCPQPEHVIGLTPHSDAGGLTILLQLNEVDGLQIKKDKIWVPIKPLPNAFVVNIGDILEIMTNGIYRSVEHRATINSSKERLSVAAFHSPKGDTLIGPMVSLITPETPALFRTIGYQDYMKKFMSRKLDGKSLVNSMRIGEGDEDK

SEQ ID NO: 100 - protein, 356 aa, Papaver bracteatum
  REGION 1..356: note = PbrDIOX-ZSNV-2004018
METPTLMKLGNGLSVPSVQELAKATLAEIPSRYICTDENLLTMGASTTDNETVPVIDLQNLLSPEPVIGMLELDRLHSACKEWGFFQLVNHGVDALLVDNEVQGFFNLPMDEKTKYGQKDGDDEGFGQFFVISEDQKLDWADVFYMSTLPLHSRKPHLFPELPLPLRETMESYSSEMKKLSMVLFDMMGKALQVVEIKGITELFEDGAQQIRMNYYPPCPQPELVFGLTSHSDFDGLTILLQLGEVEGLQIKKEERWISIKPLPDAFIVNVGDILEIMTNGIYRSVDHRAVVNSIKERLTIATFHDPRLEAEIGPISSLITPETPALFKRGVFEDLLKEMFLRKLDGKSFLDCMRM

SEQ ID NO: 101 - protein, 350 aa, Papaver rhoeas
  REGION 1..350: note = PrhDIOX-MVTX-2001522
GNGLSVPSVQELAKQTLAEIPSRYICTDENPLITGASVVDDETVPVINLQNLLSPEPVIGKLELDKLHSACKEWGFFQVVNHGVNDSLVDSVKSEIEGFFNLPANEKLKYGQKDGDVEGFGQHFVVSEDQKLDWADVFYMVTLPVRLRKPHLFPELPLPLRDTLDSYSSELNKLSMVLLEMMEKALKLVECKGITDFFEDGFQQMRMNYYPPCPRPELVTGLTSHSDFGGLTILLQLNDVEGLQIKKEERWISIKPLPNAFIVNIGDVLEIMSNGIYRSVDHRAVINSTKVRMSVATFHDPRLEAVIGPISSLITPETPALFKRGVFEDLLKEMFLRKLDGKSFLDCMRI

SEQ ID NO: 102 - protein, 351 aa, Papaver setigerum
  REGION 1..351: note = PseDIOX-JSVC-2005842
LMKLANGMSVPIVQELAKLTVGEIPSRYICTDGNLLTMGASVIDYETVPVIDLQNLQSREPVIEKLELDRLHSACKEWGFFQLLNHGVDASLMDNVRSEIRGFFNLPISDKMKYGQKDGDEEGFGQHFIVSEDQKLDWVDAFMMFTLPLHSRNPRLTPEFPQPLRETVESYSSEMKKLSVLLFELMEKALQVKGITEMFEDGLQSIRMNYYPPCPRPELAIGLTSHSDFDGLTILLQLNEVEGLQIKKEERWISIKPLPNAFIVNVGDVLEVMTNGIYRSVDHRAVVNSTKERLSIATFHDPELESEIGPIASLITPETPALFKRGRFKDLLKENLSTKLDGKSFLDCIRM

SEQ ID NO: 103 - protein, 497 aa, Homo sapiens
  REGION 1..497: note = CYP2D6
MGLEALVPLAVIVAIFLLLVDLMHRRQRWAARYSPGPLPLPGLGNLLHVDFQNTPYCFDQLRRRFGDVFSLQLAWTPVVVLNGLAAVREALVTHGEDTADRPPVPITQILGFGPRSQGVFLARYGPAWREQRRFSVSTLRNLGLGKKSLEQWVTEEAACLCAAFANHSGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLRIVVADLFSAGMVTTSTTLAWGLLLMILHPDVQRRVQQEIDDVIGQVRRPEMGDQAHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVTPSPYELCAVPR

SEQ ID NO: 104 - protein, 405 aa, Sorangium cellulosum
  REGION 1..405: note = 14HC_P450_1
MVDQDAFPELFHPSSRAEPHAIYARMRAAGRLHRLVHPRLDVPIWVAVRYDDCVELLKDPRLIRDFRKLPDEVRRRYFPLSDRTTFMDQHMLDADPPDHTRLRAIVQRAFSPRMMEGLRPRIQEIADGLIDAVIDRRRMELIADFAFPLPTAVIAELLGLPVEDRGRFRRWTKILLAPAKDREFVERAQPVVEEFAAYFRALADARRKAPRDDLISGLLLAEEQEHKLSPAELSSMVFLLLVAGHETTVHLIASGMLLLLSHPAERRRLDEDPGLVGSAVEEALRCEGPAELSTIRWSLEDIELFGARVPAGEGVAAGLLAANRDPQHFPDPDRFDIGRSPNRHIGFGGGIHFCLGAMLARIEAAIAFSTLLRRLPRIELATSTRDIVWSEWPTIRGPAAVPVVF

SEQ ID NO: 105 - DNA, 1218 bp, Sorangium cellulosum
  source 1..1218: mol_type = genomic DNA
  misc_feature 1..1218: note = 14HC_P450_1
atggtggaccaggatgcttttcctgagctttttcatcccagctcacgtgccgagccccatgcgatatacgccaggatgagagcggctgggaggttgcacagactagtgcatcctaggcttgatgtgccgatctgggttgcagtacgttatgacgactgcgtggagctattgaaagatcctagactaattcgtgattttcgtaaattacccgacgaagtccgtcgtagatactttccgttaagtgatcgtacgactttcatggaccaacatatgttggatgctgatcccccggaccataccagactgagagcaatcgtacaaagagcgttctccccgagaatgatggagggcttacgtcctcgtattcaagaaatcgcagatggactaatagatgctgtaattgatcgtagaaggatggagcttattgcggactttgcgttcccacttcccacggccgttattgcggaactgttgggcctacctgtggaggataggggcagattccgtcgttggactaaaattcttctagctcctgcgaaggatcgtgagttcgtagagagagcccaaccagtggttgaggagtttgccgcctactttcgtgcgttggcagacgctaggaggaaggcacctcgtgatgatctgatctccggactgcttctggcagaagaacaggagcataaactaagcccagcggaattaagtagcatggtattccttctgttagttgcaggtcatgaaacgaccgtacacttaatcgcgtcaggcatgcttctattgctttctcacccggctgaaaggagaaggttggacgaggacccaggcctagttgggtcagccgtcgaggaggcgttacgttgtgagggtccagcagagttgtccactatcaggtggtcacttgaggatatagagttgttcggcgccagagtgcctgccggcgagggggttgcagctggcttactagcggctaatcgtgacccccaacatttcccagaccccgatagatttgatattgggagatcacctaatagacacatcggttttggcgggggaatacacttttgtcttggtgctatgttggctaggatagaagccgcaatcgctttcagtacgttattaaggcgtttgccccgtattgagcttgcaacatctacccgtgatatagtttggagcgagtggcccactatccgtgggcccgcagcggttccggtggttttttga

SEQ ID NO: 106 - protein, 539 aa, Absidia glauca
  REGION 1..539: note = 14HC_P450_2
MPIVNALQTYSYEEPINFLKDVWLNRLFPAGTSNRKKAALLTLGATIALVCRTVYRLYRVPRSLRHIPAVGYVAMLRSILKREDATTRAMTIFQPAMKKGNGVFLVNFPLEWSIYVAEPLAAKAVLMKSDNFPKSIDFIHALGKENPVVKFFGTDNVALVNGEQWKRQRKVMNPAFHRAMPVQMFGRLLQKGFKNIEEQGHQVAALDFFQRLTLDALGHAVFGFDFGALNDREAVWTVTYESIRLNLRNPLAFAFPSMDWLLKYVIPGRLQMAASVDKLNGLMMDMIKEKRIKLLESQAQDDTPENEKDLLTLMLEAEQRGEGTTNDEELRSNMAVFFLAGHETTANTMSFCLYNLAMDKSVQEKARQEVIKVLGDEPEDVLPRNEELRQIPYLDMILKENLRRFGPASMLNARKTVEDFDMNGTFIPKNTSVIVETNALHHNPEVWKNPEQFDPERFAPGGEHETSHEGMAWLPFSSGSRGCLGMNFSMAEQRVFLAMLLRKYEWDLPKDSIHKNGIQIGNFQNACPDSLKIQFTPRY

SEQ ID NO: 107 - DNA, 1620 bp, Absidia glauca
  source 1..1620: mol_type = genomic DNA
  misc_feature 1..1620: note = 14HC_P450_2
atgcctatagtaaacgcccttcagacatattcatatgaggaaccaatcaactttcttaaggatgtatggttaaaccgtctgttcccggctggcacatccaacaggaagaaagcagcactactaacgctaggcgcgactatcgcgttggtatgtagaacggtgtacaggttgtatcgtgttccgcgtagtttaaggcacatcccagcggttggatacgtggctatgctaagaagtattcttaagcgtgaggatgcgacaaccagggctatgacgatttttcaacccgccatgaaaaagggtaatggggttttcctagtaaatttccccttggaatggtctatttatgtggcagaacccttagctgctaaagccgtcttgatgaagtccgacaatttcccgaaatccattgacttcatccatgcgttgggtaaggagaacccagtcgttaagtttttcggcaccgataacgtggcacttgtcaacggggaacagtggaaaaggcagaggaaagttatgaaccctgcatttcatagagccatgccagtacagatgttcggcaggttgttgcagaaaggatttaaaaatattgaggaacaaggccaccaagtggcggccttagacttttttcaacgtcttacgctggatgcgttaggacatgctgtgttcgggttcgacttcggggctttgaatgatcgtgaagctgtgtggacggtaacttatgagtcaatcagactgaacctaagaaatcctctggcatttgcgttcccttccatggactggcttcttaaatatgtgattcccgggagattacagatggcagcgtccgtagacaagttgaatggtctaatgatggatatgataaaagaaaaaaggattaagttgctagagtcacaggctcaggacgacactccggagaatgaaaaagatttgcttacgctgatgctggaagcggaacagagaggcgagggaaccactaacgacgaagaacttagaagcaatatggccgtctttttcttggctggacatgagacaaccgccaataccatgtccttctgcttatacaatttagcaatggacaagagtgtgcaggagaaggcgagacaggaggtaataaaggtcctaggggatgagccagaggatgtattgccacgtaacgaagagctaagacaaatcccgtatctagatatgatattaaaggagaatcttcgtaggttcggtcctgcgtctatgcttaatgccaggaagaccgtggaagactttgatatgaatggcaccttcatcccgaagaatacgtctgtaattgtcgaaacaaacgcgcttcatcacaatcctgaagtatggaagaacccggagcaattcgatccggaaagatttgctccaggtggagagcacgagaccagccacgaagggatggcttggttgcctttttcatctggtagcaggggttgtttgggaatgaacttttctatggcggaacagcgtgtcttcttggcaatgctgctgaggaaatacgagtgggatctaccaaaggatagcattcataagaacggtatccagattggtaactttcaaaacgcttgccctgacagtctgaagatccaatttacgccgcgttattag

SEQ ID NO: 108 - protein, 525 aa, Choanephora cucurbitarum
  REGION 1..525: note = 14HC_P450_3
MFTETALQIYHQMAEKVLPILKRQPKSSYIGAALVFLLVAEIRRQLSVPKHLKKFPTIGVFTLMKSFMRNDSVIERHNRLVAPIVKQGHKFYAAKIPFDWSLSIVDPEIAKIMLMKSDAFPKSQGFTRKLGDSSLIVRFTGRDNVSISNGHVWKRQRKFMNPAFRRSTPIKTFGELTLKFFDCVDEQPQDFPAAIKLKNYTLDALGIAAFDFDFKSLSGDPEGWTEIYNVIMKGMFDPWVFLFGKMEFILQYIIPSKRECIKSVVKFNKMLVEMADKRRQEIQNGKKLNTPNSEKDLLTLMIEAEMEEGIMTTNEELRENIALFFLAGQDSTGNSLSFCLYHLAKNKHVQDKLRREIMSVMGDRDLDAIPTVEDFKDMPYLNMVIKENLRLSGPADRFLDRVVAEDIVLGGELIPKGTLITVDVASIHYNPEYWHDPEVFIPERFEPNGEFDQHAGVAWLPFSNGARQCIGMNFSLAEQRVFLTMLLRRYEVGISKDSIHYDSIVYEQSFTFAPSSLTLNFTKLN

SEQ ID NO: 109 - DNA, 1578 bp, Choanephora cucurbitarum
  source 1..1578: mol_type = genomic DNA
  misc_feature 1..1578: note = 14HC_P450_3
atgttcacagagactgcgttgcagatctatcaccaaatggcggaaaaagtgctacccattttgaaaagacaacccaaatcttcttatatcggcgcggcgttagtcttcctgcttgtagccgaaataagaaggcagctatctgtacctaaacacctgaagaagtttccgacgattggggttttcacgttaatgaagagctttatgaggaatgactcagtgatcgaaaggcataataggttggtggcacccattgtgaagcaaggtcataagttttacgcggcaaagattccttttgattggtctttgtcaattgtggacccagagatcgccaagataatgcttatgaaatctgatgctttccccaaaagccagggtttcaccaggaaactgggtgactcatcactgatcgtgaggtttactggcagggataatgtgagtatttctaatggacacgtttggaaaagacagagaaagttcatgaatcccgcgttcaggcgttctacacccattaagaccttcggggaattaacgctaaaattcttcgattgtgtagatgaacaaccgcaagatttccctgccgcaattaaactaaagaactacacacttgatgcgctgggaattgcggcattcgatttcgactttaaaagtctatctggcgatcccgaaggatggacagaaatttacaacgtgatcatgaagggtatgtttgatccatgggtattcttgttcggtaagatggagtttattctacagtatatcataccatcaaaacgtgagtgcattaaatctgtcgttaagttcaataaaatgctagtcgaaatggcagacaaaagacgtcaagaaatccagaatggaaaaaaattgaacacgcccaatagtgaaaaagacctgctaactttgatgatagaagcggaaatggaagagggtataatgactactaatgaagagcttcgtgagaacatcgccctatttttcttggccggtcaggacagtactggtaacagtctgagtttctgtttatatcatttggcaaaaaataaacatgtacaggacaaactaagaagggagatcatgtctgtgatgggggatagagaccttgatgcaatacccacggtggaagacttcaaagacatgccttatcttaatatggtaatcaaagaaaacttacgtttgtctggccctgcggatcgtttcctagatagagttgtagcggaagatatcgtgcttgggggtgagttaatcccaaaaggaaccttaataacagttgatgtagccagtattcattacaatcctgagtactggcatgacccggaggtcttcatccccgaaagattcgagccgaacggtgagttcgatcagcacgctggtgtggcgtggctaccattctctaatggagcccgtcaatgcataggtatgaatttcagtcttgctgagcagagagtgtttctgactatgttattgcgtcgttacgaggtgggtatcagtaaagatagcatccattacgatagcatagtatatgagcaatcatttacgtttgctcctagtagccttacgctgaattttacgaagctgaactag

SEQ ID NO: 110 - protein, 534 aa, Lichtheimia corymbifera
  REGION 1..534: note = 14HC_P450_4
MYTLVSFLKDQNIIAVLKNAIHAQQQGTTASSITILSLAVAITTFAVHRIRLYSVKEHGVPLVPYVLPFIGSSPEYRKDPKAFLEKWTAKFGPVFRAHIFGRVYTIISGHYVREVFLNEDFSFEVGMGKTFDGWLITDTKKSELFSTPLVRSMVMKHLAVDVKNYTPRAVEHLTIAAREMLGDIGESKELPHLYPLIQHMVSTAIASILVGLKICKDKDLLETFKNVAVDIGSELNPDSYLYEAFPTISRLRQWYLGKYGKAINKHQEHMLRVLGPEIDERLAAMERGDGGWERPEDILQGILETAKLTSDHPQRYMVPIKWFLILVFASIHTTSQNTTVAMYRLLQHPEVIDELLEEQNQVFEKHHGSNYDDHDITKLLTGEVIKDLVKLDSVCREAMRISSFYAELPHTYIGKSPLTMSNGTIINPGDDVLINGYTNHHDPDIQIDGGGDYAEFKPFRFVEKGRQSTRIGDDYLIFGQGKHACPGRWFAMQEMKTTISFLIRQYIITAKGDIMFLKGHRQKIPMGQVIFQKR

SEQ ID NO: 111 - DNA, 1605 bp, Lichtheimia corymbifera
  source 1..1605: mol_type = genomic DNA
  misc_feature 1..1605: note = 14HC_P450_4
atgtataccctagtctcattcttaaaagaccaaaacatcattgcggtgttgaaaaatgccattcatgcacaacaacaaggcaccaccgcaagttctattactattctttctctagctgttgcgataacgactttcgctgttcatcgtattagactttactccgttaaggagcatggtgttcccctggtaccgtatgtcctaccctttattgggtcctctccagagtacagaaaagacccaaaggccttcctagaaaagtggaccgctaaattcgggccagtgttccgtgcgcatatctttggcagggtgtacacaataatatccggccattacgtcagggaggttttcttgaatgaagatttcagctttgaagttggtatgggtaaaacgttcgacggctggcttataaccgacacaaagaaatctgagctgttttccactccacttgtacgtagtatggtcatgaagcatctggccgttgatgtcaagaactacacgccgagagcagtggaacatcttaccattgccgctagggagatgctgggtgacatcggtgaatcaaaagagctgccacacttatatcccctgattcaacatatggtaagcacggcgatagcctctatcttggtgggccttaaaatctgtaaagacaaagatctacttgagacctttaagaacgtggccgttgatataggctctgaattgaatccagactcctacttgtatgaagcgtttcctaccataagcaggttgagacagtggtacctgggaaagtatgggaaggcaatcaacaaacaccaggaacacatgctgagagtactgggacctgagatagatgaacgtctagcggccatggagcgtggagatggtggctgggaaaggcccgaggacattcttcaagggattttagagaccgccaaactgacaagtgatcaccctcaacgttacatggttcccatcaaatggtttctgatattggtttttgcaagtatccacacgacctcacagaatacaacggtagctatgtacaggcttttgcaacatccggaagttatagatgagttacttgaagaacaaaaccaggtcttcgagaagcaccacgggagcaattatgatgatcatgacataactaagttgctaacgggggaagtcataaaagatttggtcaagttagactcagtctgccgtgaagcaatgcgtatatcatctttctacgctgaattgcctcacacatacattggcaagagtccattaactatgagtaacggaacaatcatcaaccccggagacgatgtgctgattaatggctacaccaaccaccatgaccccgacattcagattgacgggggtggcgactatgcagaattcaagcccttcaggtttgtcgagaaaggcaggcagtctacaagaatcggagacgattatcttatcttcggacaaggcaaacacgcatgtccaggaaggtggtttgccatgcaagaaatgaagactactatctccttcttgatacgtcaatatattatcacagccaagggtgatataatgttcctaaagggtcacagacaaaaaatccctatgggacaggtgatttttcagaagcgttag

SEQ ID NO: 112 - protein, 514 aa, Lichtheimia ramosa
  REGION 1..514: note = 14HC_P450_5
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTLDALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 113 - DNA, 1545 bp, Lichtheimia ramosa
  source 1..1545: mol_type = genomic DNA
  misc_feature 1..1545: note = 14HC_P450_5
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgttagacgcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 114 - protein, 523 aa, Mucor circinelloides
  REGION 1..523: note = 14HC_P450_6
MTLIASYNQTLLERVISVVRKNKTSYIGMAILCVVLQQVYASFTVPPKYLRRYPTVSFFEMMKSFYRKESVLNRNKRLVTPLTNAGHGFYVCRIPLDWTIYVTDPIAAKTLLLKTDNFPKSHAIFGALGESSPAVKFMGSENVAVSNGEMWKKQRKIMNPAFHRSQPVKLFGGVMPDLFALIDQDPEHVFVAPRMKSFALDALGLSAFGFDFQSLKGDPEGWTSKYNTVISTLFNPFINLFAKYDFLIKYISPERRRVIKATDEFNVMLSNLADKRRQEILDGEKKDMAENEKDLLTLMIEADIREGVETTTTELRHNMAIFFLAGHDTTANTLALCLYQLAKHKHVQKKARQEVLDILGDDPCDVTPTLEDLKKLNYVNMVIKENLRRNSPVDNLMARDTHQDIDLNGTFIPKGSKVTVNVASIHLNPKIWHDPESFIPERFEPGGEYDGHDGFTWIPFSNGSRQCLGLNFSLTEQRVVLCMLLKRYEIEIPKDSIHYNEIVFDKPFTFAPQSLELSFKKRY

SEQ ID NO: 115 - DNA, 1572 bp, Mucor circinelloides
  source 1..1572: mol_type = genomic DNA
  misc_feature 1..1572: note = 14HC_P450_6
atgaccctgatagcttcttataatcagacactgcttgaacgtgttataagcgtggttaggaagaacaagacctcctatatcggaatggcgattttatgcgtggttctgcagcaagtgtatgcgtcttttacggtgccgcctaaatatttaaggagatacccgacagttagcttttttgaaatgatgaagagcttttaccgtaaagagtcagtgctgaaccgtaataagcgtctggtaaccccccttactaacgccgggcatggtttctatgtgtgtagaatcccattggattggaccatttatgttaccgatcctatagcagctaagaccctactgcttaaaacagacaactttcctaaatctcacgcaatatttggcgcgttaggcgaatcctcccctgcagtcaagttcatgggctctgaaaatgtagctgtatctaacggcgaaatgtggaaaaagcaaagaaaaataatgaaccccgcgtttcacagatcacagcccgttaagcttttcggtggcgttatgccggatctattcgctctgattgaccaggaccctgagcatgtctttgttgcgccgcgtatgaaaagttttgcgcttgatgcccttgggctgtcagccttcggttttgattttcaaagtctgaagggagaccctgagggttggaccagcaagtacaataccgtgatatctacgctatttaacccttttatcaaccttttcgctaagtatgacttccttattaagtacatttctccggaaaggcgtcgtgtgattaaagctacggacgagtttaatgtaatgctgtccaatctagccgacaagagaaggcaggaaatcttggacggggaaaagaaagacatggccgaaaatgaaaaggacctattgacattaatgattgaagcggatataagagagggagttgaaacaactacaacggagctgagacacaatatggctatattttttcttgctgggcacgacactaccgccaataccctggcattatgtttatatcaacttgccaaacataagcatgtacaaaaaaaagctagacaagaggtgttggacatcctaggagacgacccatgcgacgttaccccgactctagaggacttgaagaagcttaactacgtgaacatggtgataaaggaaaacctgcgtcgtaattcccccgtggacaatttgatggccagggacacacaccaagacatagacctgaatggtacttttatcccgaaaggaagcaaagtcactgttaatgtagcgagtatccatctgaatcccaaaatttggcatgatcccgaaagtttcataccggagaggttcgagccaggcggggaatatgatggtcatgatggattcacctggattccgtttagtaatgggagtcgtcagtgcctggggctgaattttagtttaacagagcagagagtagtcttatgtatgttgctaaagagatacgaaatcgagataccaaaagattcaatacattacaatgagattgtcttcgataagcccttcacgttcgcgccccagtccttggaattaagctttaaaaagcgttattag

SEQ ID NO: 116 - protein, 522 aa, Rhizopus microsporus
  REGION 1..522: note = 14HC_P450_7
MDHLIQVYNSSTQVLIPVLQKRSKASYITAAIALIIAQRLYSYFRVPKHLRGFPKLPYFGIAKSFFAKESPRERVKKYILPIINERDGFYISNIPFGWMLYVTNPIAAKQILLKSNGFPKNHGLLEDMGENLFIEFIGKDNVVLTNGDTWKRQRKVMNPAFHHSLPIKTMSNVVFSLISVIDQANGTVPVASTMQNFTLDTLGLAIFGFDFKALQGDGDEWTKTYRLVSDCLFDPIINVFSSYSFIFDRIYPRRRRGAMATRKLGEKFLEIAQQKRMEIKSGAFADVPDNEKDLLTLMLEAEEKGDVWTSEDELRHNIAVLFLAGHDTTAHALSFCFYHLAKNKDIQQKLRKEVLDLLGDEPVDVVPTVEQLKDMQYLNMVIKENLRMNSPADMLFSRDVQEDIVLANTFIPKGTVISINIEALHCNPKLWHNPDQFDPERFAPGGEHEQHEGMTWLPFSNGTRQCLGMNFSLFEQRLVIAMILKKYEISIPEDSIHRNHIINDMPFNVAPKSLELTFTKRY

SEQ ID NO: 117 - DNA, 1569 bp, Rhizopus microsporus
  source 1..1569: mol_type = genomic DNA
  misc_feature 1..1569: note = 14HC_P450_7
atggatcatttaattcaagtatacaatagttctacacaagtattgattccagttcttcaaaagagatcaaaggcatcttacattacagccgcgattgccttaataatcgctcaacgactgtactcttactttcgtgtacccaagcatcttcgtggcttccccaaattaccttactttggaatagcaaagtcattctttgcaaaagaatcaccaagggaaagagtgaagaaatatatattacctattattaatgaacgcgatggattttatattagtaatatcccttttggatggatgctctacgtcactaaccctatagcagccaagcagattctactcaaatcaaatgggtttcccaagaatcatggactactagaagatatgggtgaaaatctatttatagaattcattggcaaagataatgtagttcttactaatggagatacctggaagaggcaaagaaaggtcatgaaccccgctttccatcactctttgcctatcaaaaccatgtctaacgttgtgttctcattgatatcagtcatcgaccaagcaaacggtacagttccggttgcatctaccatgcagaattttactcttgatacacttggtcttgctatctttggctttgactttaaagcattacagggcgatggggatgaatggaccaagacttacagacttgtaagcgactgtctctttgaccctattatcaatgtcttcagctcgtactcttttatctttgaccgcatctatcctagacgacgaagaggcgctatggctactagaaaacttggtgaaaaattcttggagattgcgcaacaaaagcgcatggaaatcaaaagtggtgcttttgcagatgtaccagacaatgaaaaggatcttttgacacttatgcttgaggctgaagaaaagggcgatgtatggacatcagaagacgagcttagacataatattgcagtcttgtttttagctggacatgataccactgctcatgctctgtccttctgtttctaccatcttgcaaagaataaggatattcaacaaaagctaagaaaagaagtgcttgatcttctgggcgatgagccagttgatgtagtgcctacagttgagcaactcaaagatatgcaatacctcaatatggtcataaaagagaatctgcgtatgaatagcccagctgatatgctattttcacgagatgtacaagaagacatcgttttggccaacacgttcattcccaagggtactgttatttcaatcaatatagaggctttgcactgcaaccccaagctttggcataacccagaccaatttgatcctgaaagatttgctcctggtggtgaacatgaacaacatgaaggtatgacttggctaccttttagtaatggaacccgtcaatgccttggcatgaatttctctctgtttgagcaacgattggttattgctatgatcttgaaaaagtatgagattagtattccggaagactccattcatcgtaatcatattattaatgacatgccattcaatgtcgcgcccaagtcactggaactgacttttaccaagcgttattaa

SEQ ID NO: 118 - protein, 522 aa, Rhizopus microsporus
  REGION 1..522: note = 14HC_P450_8
MDHLIQVYNSSTQVLIPVLQKRSKASYITAAIALIIAQRLYSYFRVPKHLRGFPKLPYFGIAKSLLTRESPRERVKKYVLPIIDENDGFYISKIPFGWMLYIANPVAAKQLLLKSSGFPKNHGLLDNMGENLFVEFIGKDNVALSNGDTWKRQRKVMNPAFHHSLPIKTMSKVVFSLISAIEQANGTIPVASAMQNFTLDTLGLAIFGFDFKALQGDPDEWTKTYRFVSDCIFDPVINVFSSYSFIIDRIYPRRRRGAIATKKLSEKFLKIAQQKRKEIKSGAYADVPDNEKDLLTLMLEAEEKGDTWTSEDELRHNIAILFVAGHDTTAHALSFCFYHLAKNKDVQQKLRKEVLSILGDKPVDVVPTVEQLKNMQYLNMVIKENLRINSPADMLFSRDVKEDTILANTLIPKGTVVTINIEALHYNPRLWHNPDQFDPERFAPGGEHEKHEGMTWLPFSNGTRQCLGMNFSLFEQRLVIAMILKKYEISIPEDSIHRDHIVSDIPFNGAPKSLKLTFTKRH

SEQ ID NO: 119 - DNA, 1569 bp, Rhizopus microsporus
  source 1..1569: mol_type = genomic DNA
  misc_feature 1..1569: note = 14HC_P450_8
atggatcatcttattcaggtgtacaacagctcaacgcaagttttaattccggttcttcagaaaaggtctaaggcgagttacattaccgccgcaatagcgctgattatagcacagaggctatattcttactttcgtgtacctaaacatctgcgtggctttcccaaattaccgtacttcggcatcgccaaatcactactgactagggaaagcccaagagagagagtcaagaagtacgtattacctataatcgacgagaacgatggtttctacataagtaaaattccctttgggtggatgctgtatatcgcaaatccggtggctgcgaaacaacttttgctgaagtcttcagggttccccaagaaccacggcttattagataacatgggcgaaaatttattcgtggagtttattggaaaggacaacgtcgctcttagcaatggggatacgtggaagaggcagagaaaagtcatgaacccagccttccaccattctttgcctattaagaccatgtctaaagtagtgttctcactgatctcagcgattgaacaggcaaatggcacaatccccgtcgcgtcagctatgcagaattttacgctagacactttggggttggctattttcgggtttgacttcaaagccttacaaggagatcccgacgaatggacaaaaacataccgttttgtctctgactgtatttttgaccccgttatcaacgtcttttcttcctacagtttcataatcgaccgtatatatcctagaagaaggcgtggcgcaattgcaacgaaaaagttaagtgagaaattccttaaaatcgcccaacaaaaaaggaaggagataaaatctggagcctacgccgacgtgcctgataacgagaaagatttgctaacgctgatgttggaagcagaggaaaagggcgacacttggaccagcgaagatgagcttagacacaatatcgcgatccttttcgtcgcgggccatgatacaacggcccacgctctatcattctgtttttatcatctggccaagaacaaagacgtacaacagaagctgaggaaggaggttctaagtatcctaggggataagcccgtggatgttgtccccaccgtggagcaattaaagaacatgcagtacctgaatatggtgataaaagaaaatcttaggattaactcacccgccgatatgttattcagcagggatgtcaaggaggacacgatattggctaacactctaatcccgaaagggacagtggtcacgatcaatatcgaagccctacattacaatccgaggttatggcataacccggatcagttcgatccggaaaggttcgctcctggaggcgaacatgagaaacacgaaggcatgacatggttaccattctccaatggaacacgtcagtgtttaggaatgaattttagcttgttcgagcaaagattagttatcgccatgattttgaagaagtacgagatatccatacccgaggattctattcatagggaccatatagtgagtgatataccctttaacggtgctcctaaatctctgaagctaacttttactaagaggcactag

SEQ ID NO: 120 - protein, 502 aa, Homo sapiens
  REGION 1..502: note = 14HC_P450_9
MDLIPNLAVETWLLLAVSLVLLYLYGTRTHGLFKRLGIPGPTPLPLLGNVLSYRQGLWKFDTECYKKYGKMWGTYEGQLPVLAITDPDVIRTVLVKECYSVFTNRRSLGPVGFMKSAISLAEDEEWKRIRSLLSPTFTSGKLKEMFPIIAQYGDVLVRNLRREAEKGKPVTLKDIFGAYSMDVITGTSFGVNIDSLNNPQDPFVESTKKFLKFGFLDPLFLSIILFPFLTPVFEALNVSLFPKDTINFLSKSVNRMKKSRLNDKQKHRLDFLQLMIDSQNSKETESHKALSDLELAAQSIIFIFAGYETTSSVLSFTLYELATHPDVQQKLQKEIDAVLPNKAPPTYDAVVQMEYLDMVVNETLRLFPVAIRLERTCKKDVEINGVFIPKGSMVVIPTYALHHDPKYWTEPEEFRPERFSKKKDSIDPYIYTPFGTGPRNCIGMRFALMNMKLALIRVLQNFSFKPCKETQIPLKLDTQGLLQPEKPIVLKVDSRDGTLSGE

SEQ ID NO: 121 - DNA, 1509 bp, Homo sapiens
  source 1..1509: mol_type = genomic DNA
  misc_feature 1..1509: note = 14HC_P450_9
atggaccttatcccaaacctggcggtcgagacttggttacttctggccgtaagcctagttttgctgtatttgtatggtactcgtactcacggtttattcaagagactaggtattccagggcctactcccttacctctgttaggaaatgttctgtcttacagacaaggcctatggaaattcgatacggagtgctacaagaaatatggcaaaatgtggggcacctacgagggacagcttcccgtgctggcgattacggatcctgacgtaataagaaccgtgttggtaaaagaatgctacagtgtattcaccaaccgtaggtccttaggacctgtcggattcatgaaatccgcgataagcctagcagaggacgaagaatggaagaggatcagatccttattaagcccaacgtttacatcaggaaagttgaaagaaatgttccccattattgctcagtacggtgatgtcctggtcaggaatttgaggcgtgaagctgagaagggtaaacccgttacacttaaagacatctttggagcttactccatggacgtcatcaccggcacatctttcggtgtcaacatcgatagtcttaacaatcctcaagatccctttgtggagagcacaaagaagtttctaaaatttgggtttttggaccctctatttctatctataatacttttcccgtttcttacgcctgtcttcgaggctttaaatgtctcactttttccgaaggataccattaatttcttgagtaaatcagtcaatcgtatgaaaaaatcacgtttgaatgacaagcagaaacatcgtctggactttttacaactgatgatcgattctcagaattccaaggaaactgagtctcacaaggcgctttcagatttggagctagctgctcagagtataatttttatctttgccgggtatgagacgactagctctgtcctgtcatttactctttacgagttagcaacgcatcctgatgttcaacaaaaacttcagaaagaaattgatgctgtactaccgaacaaagcgccgcccacttacgatgcagtcgtgcaaatggaatatcttgatatggtggtaaacgagacactgaggctattcccagtagctattaggttggagcgtacttgtaaaaaggatgtcgagattaacggagtttttatacccaagggctcaatggtggttattccgacgtatgcacttcaccacgatcctaaatattggacagaaccagaagaattcagaccggagaggttctcaaaaaagaaagactcaatcgacccatacatatatacaccctttggcacgggtccgaggaattgcattggtatgaggtttgctttgatgaatatgaaattagctttaataagagtactgcaaaatttttccttcaaaccctgcaaggaaacccagataccacttaaactggacactcagggtctgctgcagcccgaaaagccaatagtgttgaaggtagattctcgtgacggcaccttgagtggagaatga

SEQ ID NO: 122 - protein, 516 aa, Homo sapiens
  REGION 1..516: note = 14HC_P450_10
MALSQSVPFSATELLLASAIFCLVFWVLKGLRPRVPKGLKSPPEPWGWPLLGHVLTLGKNPHLALSRMSQRYGDVLQIRIGSTPVLVLSRLDTIRQALVRQGDDFKGRPDLYTSTLITDGQSLTFSTDSGPVWAARRRLAQNALNTFSIASDPASSSSCYLEEHVSKEAKALISRLQELMAGPGHFDPYNQVVVSVANVIGAMCFGQHFPESSDEMLSLVKNTHEFVETASSGNPLDFFPILRYLPNPALQRFKAFNQRFLWFLQKTVQEHYQDFDKNSVRDITGALFKHSKKGPRASGNLIPQEKIVNLVNDIFGAGFDTVTTAISWSLMYLVTKPEIQRKIQKELDTVIGRERRPRLSDRPQLPYLEAFILETFRHSSFLPFTIPHSTTRDTTLNGFYIPKKCCVFVNQWQVNHDPELWEDPSEFRPERFLTADGTAINKPLSEKMMLFGMGKRRCIGEVLAKWEIFLFLAILLQQLEFSVPPGVKVDLTPIYGLTMKHARCEHVQARLRFSIN

SEQ ID NO: 123 - DNA, 1551 bp, Homo sapiens
  source 1..1551: mol_type = genomic DNA
  misc_feature 1..1551: note = 14HC_P450_10
atggccttgtctcaatctgttccattttctgctactgagttgttgttggcttctgctattttctgtttggttttctgggtcttgaagggtttaagaccaagagttccaaaaggtttgaaatctccaccagaaccatggggttggccattattgggtcatgttttgactttgggtaagaacccacatttggctttgtctagaatgtctcaaagatacggtgatgtcttgcaaatcagaattggttctactccagttttggtcttgtctagattggataccattagacaagccttggttagacaaggtgatgattttaaaggtaggccagacttgtacacctctactttgattactgatggtcagtctttgaccttctctactgattctggtccagtttgggctgctagacgtagattggctcaaaatgctttgaatactttctcgattgcttccgatccagcttcttcttcatcttgttatttggaggaacacgtgtccaaagaagctaaggctttgatttctaggttgcaagaattgatggctggtccaggtcattttgatccatacaatcaagttgttgtctccgttgctaatgttattggtgctatgtgtttcggtcaacactttccagaatcttcagacgaaatgttgtccttggttaagaacacccacgaatttgttgaaactgcttcttctggtaaccctttggatttctttccaatcttgcgttacttgccaaatccagcattgcaaagattcaaggcttttaaccagagattcttgtggttcttgcaaaagaccgttcaagaacactaccaagatttcgataagaactccgttagagatattactggcgctttgtttaagcactctaagaaaggtccaagagcctctggtaatttgattccacaagaaaagatcgtcaacctggttaacgatatctttggtgctggtttcgatactgttactactgctatttcttggtccttgatgtacttggttaccaagccagaaattcagaggaagatccagaaagaattggacaccgttattggtagagaaagaaggccaagattgtctgatagaccacaattgccatatttggaagccttcattttggaaactttcaggcactcttctttcttgccattcactattccacattctactactagagataccactttgaacggtttctacatcccaaagaaatgctgcgttttcgtcaatcaatggcaagttaatcacgatccagaattgtgggaagatccttctgagtttagaccagaaagattcttgactgctgatggtacagctattaacaaaccattgtccgagaagatgatgttgtttggtatgggtaagagaagatgcatcggtgaagttttggctaagtgggaaattttcttgttcttggccattttgttgcagcagttggaattttcagttccaccaggtgttaaggttgatttgactccaatctatggtttgactatgaagcacgctagatgtgaacacgttcaagctagattgagattctccatcaactaa

SEQ ID NO: 124 - protein, 494 aa, Homo sapiens
  REGION 1..494: note = 14HC_P450_11
MLASGMLLVALLVCLTVMVLMSVWQQRKSKGKLPPGPTPLPFIGNYLQLNTEQMYNSLMKISERYGPVFTIHLGPRRVVVLCGHDAVREALVDQAEEFSGRGEQATFDWVFKGYGVVFSNGERAKQLRRFSIATLRDFGVGKRGIEERIQEEAGFLIDALRGTGGANIDPTFFLSRTVSNVISSIVFGDRFDYKDKEFLSLLRMMLGIFQFTSTSTGQLYEMFSSVMKHLPGPQQQAFQLLQGLEDFIAKKVEHNQRTLDPNSPRDFIDSFLIRMQEEEKNPNTEFYLKNLVMTTLNLFIGGTETVSTTLRYGFLLLMKHPEVEAKVHEEIDRVIGKNRQPKFEDRAKMPYMEAVIHEIQRFGDVIPMSLARRVKKDTKFRDFFLPKGTEVYPMLGSVLRDPSFFSNPQDFNPQHFLNEKGQFKKSDAFVPFSIGKRNCFGEGLARMELFLFFTTVMQNFRLKSSQSPKDIDVSPKHVGFATIPRNYTMSFLPR

SEQ ID NO: 125 - DNA, 1485 bp, Homo sapiens
  source 1..1485: mol_type = genomic DNA
  misc_feature 1..1485: note = 14HC_P450_11
atgttggcttctggtatgttgttggttgctttgttggtttgtttgaccgttatggttttgatgtccgtttggcaacaaagaaaatctaaaggcaaattgccaccaggtccaactccattgccttttattggtaattacttgcagttgaacaccgagcagatgtacaattccttgatgaagatctctgaaagatacggtccagttttcaccattcatttgggtcctagaagagttgttgttttgtgtggtcatgatgctgttagagaagctttggttgatcaagccgaagaattttctggtagaggtgaacaagctactttcgactgggttttcaaaggttatggtgtcgttttctctaacggtgaaagagctaaacagttgagaagattctctattgccaccttgagagattttggtgttggtaagagaggtatcgaagaaagaatccaagaagaagccggtttcttgattgatgctttgagaggtactggtggtgctaatattgatccaacattcttcttgtccaggaccgtttctaacgttatttcctctattgtgttcggtgacagattcgactacaaagacaaagagttcctgtccttgttgagaatgatgttgggtattttccaattcacctccacttccactggtcaattatacgaaatgttctcctcagtcatgaagcacttgccaggtccacaacaacaagcttttcaattattgcaaggtttggaggacttcattgccaagaaggttgaacataatcagagaactttggacccaaactctccaagagatttcatcgattcattcttgatcaggatgcaagaggaagaaaagaacccaaacaccgagttttacttgaagaacttggttatgaccaccttgaacttgttcattggtggtactgaaactgtttccactactttgagatatggcttcctgttgttgatgaagcacccagaagttgaagctaaggttcatgaagaaatcgatagagtcatcggtaagaacagacaacctaagtttgaagatagagccaagatgccttatatggaagctgttattcacgaaatccaaagattcggtgatgtcatcccaatgtctttggcaagaagagttaagaaggataccaagttcagggacttcttcttaccaaaaggtactgaagtttacccaatgttgggttccgttttgagagatccatctttctttagcaacccacaagactttaacccacagcatttcttgaacgaaaagggccagtttaagaaatccgatgcttttgttccattctccattggcaagagaaattgctttggtgaaggtttggctaggatggaattattcttgttcttcactaccgtcatgcagaacttcagattgaagtcatctcaatccccaaaggatatcgatgtttccccaaaacatgttggtttcgctactattccaagaaactacaccatgtctttcttgcccagataa

SEQ ID NO: 126 - protein, 494 aa, Homo sapiens
  REGION 1..494: note = 14HC_P450_12
MLASGLLLVTLLACLTVMVLMSVWRQRKSRGKLPPGPTPLPFIGNYLQLNTEQMYNSLMKISERYGPVFTIHLGPRRVVVLCGHDAVKEALVDQAEEFSGRGEQATFDWLFKGYGVAFSNGERAKQLRRFSIATLRGFGVGKRGIEERIQEEAGFLIDALRGTHGANIDPTFFLSRTVSNVISSIVFGDRFDYEDKEFLSLLRMMLGSFQFTATSTGQLYEMFSSVMKHLPGPQQQAFKELQGLEDFIAKKVEHNQRTLDPNSPRDFIDSFLIRMQEEEKNPNTEFYLKNLVMTTLNLFFAGTETVSTTLRYGFLLLMKHPEVEAKVHEEIDRVIGKNRQPKFEDRAKMPYTEAVIHEIQRFGDMLPMGLAHRVNKDTKFRDFFLPKGTEVFPMLGSVLRDPRFFSNPRDFNPQHFLDKKGQFKKSDAFVPFSIGKRYCFGEGLARMELFLFFTTIMQNFRFKSPQSPKDIDVSPKHVGFATIPRNYTMSFLPR

SEQ ID NO: 127 - DNA, 1485 bp, Homo sapiens
  source 1..1485: mol_type = genomic DNA
  misc_feature 1..1485: note = 14HC_P450_12
atgttggcttctggtttgttgttggttactttgttggcttgtttgaccgttatggtcttgatgtctgtttggagacaaagaaagtctagaggtaaattgccaccaggtccaactccattgccttttattggtaattacttgcagttgaacaccgagcagatgtacaattccttgatgaagatctctgaaagatacggtccagttttcaccattcatttgggtcctagaagagttgttgttttgtgtggtcatgatgctgtcaaagaagctttggttgatcaagccgaagaattttctggtagaggtgaacaagctactttcgactggttgtttaaaggttatggtgttgctttctccaacggtgaaagagctaaacaattgagaagattctccatagctaccttgagaggtttcggtgttggtaaaagaggtatcgaagaaagaatccaagaagaagccggtttcttgattgatgctttgagaggtactcatggtgctaatattgatccaacgttcttcttgtctaggaccgtttctaacgttatctcctctattgtctttggtgacagatttgactacgaggacaaagagttcttgtccttgttgagaatgatgttgggttctttccaattcactgctacttccactggtcaactgtacgaaatgttttcctcagttatgaagcacttgccaggtccacaacaacaagcttttaaagaattgcaaggtttggaggacttcattgccaagaaggttgaacataatcagagaactttggacccaaactctccaagagatttcatcgattcattcttgatcaggatgcaagaggaagaaaagaaccctaatactgagttctacctgaagaacttggttatgactaccctgaatttgttcttcgctggtactgaaactgtttctaccactttgagatacggattcttgttgttaatgaagcacccagaagttgaagccaaggttcatgaagaaatcgatagagttatcggtaagaacagacagccaaagtttgaagatagagctaagatgccatacaccgaagctgttattcacgaaattcaaagattcggtgacatgttgccaatgggtttagctcatagagttaacaaggataccaagttcagggatttcttcttgcctaaaggtactgaagttttcccaatgttgggtagcgttttgagagatccaagattcttttcaaacccaagagactttaaccctcagcatttcttggataagaagggccaattcaagaaatccgatgcttttgttccattctccattggtaagagatactgttttggtgaaggtttggctaggatggaactgttcttgttcttcactaccatcatgcagaacttcagattcaagtctccacaatctccaaaggatatcgatgtttccccaaaacatgttggtttcgctactattccaagaaactacaccatgtctttcttgccaagataa

SEQ ID NO: 128 - protein, 490 aa, Homo sapiens [sequence omitted]

SEQ ID NO: 129 - DNA, 1473 bp, Homo sapiens
  source 1..1473: mol_type = genomic DNA
  misc_feature 1..1473: note = 14HC_P450_13
atggattccttggttgttttggtcttgtgtttgtcttgcctgttgttgttgtctctttggagacaatcttctggtagaggtaaattgccaccaggtccaactccattgccagttattggtaatatcttgcagatcggtatcaaggacatctctaagtctttgaccaacttgtctaaagtttacggtccagttttcaccttgtactttggtttgaaacctatcgttgtcttgcatggttatgaagctgtaaaagaagccttgatcgatttgggtgaagaattttcaggtaggggtattttcccattggctgaaagagctaatagaggttttggtatcgttttctccaatggcaagaagtggaaagagattagaaggttctctttgatgaccttgagaaactttggtatgggcaagagatccatcgaagatagagttcaagaagaagctcgttgcttggtagaagaattgagaaagacaaaagcttctccatgtgatccaaccttcattttgggttgtgctccatgtaacgttatctgctctattatcttccacaagaggttcgattacaaggaccaacagttcttgaacctgatggaaaagttgaacgagaacatcaagattctgtcctctccatggattcaaatctgcaacaatttctccccaatcatcgattactttccaggtactcataacaagctgttgaagaacgttgctttcatgaagtcctacatcttggaaaaggtcaaagaacaccaagagtccatggatatgaacaacccacaagatttcatcgactgtttcttgatgaagatggaaaaggaaaagcacaatcagccatctgagttcaccattgaatctttggaaaacaccgctgttgatttgtttggtgctggtactgaaactacttctactactttgagatacgccctgctgttgttattgaaacatccagaagttaccgctaaggtccaagaagaaattgaaagagttatcggtaggaacagatctccatgtatgcaagatagatctcatatgccatacactgatgccgttgttcacgaagttcaaaggtatatcgatttgttgccaacctctttgccacatgctgttacttgtgatatcaagttccgtaactacttgatcccaaagggtactaccatcttgatttctttgacttctgtcctgcacgacaacaaagaatttccaaatccagaaatgttcgaccctcatcacttcttggatgaaggtggtaatttcaagaaatccaagtacttcatgccattctctgccggtaaaagaatttgtgttggtgaagctttggctggcatggaattattcttgttcttgacctcaatcctgcaaaacttcaacctgaagtctttggttgatccaaagaacttggataccactccagttgttaatggttttgcttctgttccaccattctaccaattgtgcttcattccagtttaa

SEQ ID NO: 130 - protein, 490 aa, Homo sapiens [sequence omitted]

SEQ ID NO: 131 - DNA, 1473 bp, Homo sapiens
  source 1..1473: mol_type = genomic DNA
  misc_feature 1..1473: note = 14HC_P450_14
atggacccattcgttgttttggttttgtgtttgtcttgcctgctgttgttgtctatttggagacaatcttctggtagaggtaaattgccaccaggtccaactccattgccagttattggtaatatcttgcagatcgacatcaaggacgtttctaagtctttgaccaacctgtctaaaatctacggtccagttttcaccttgtacttcggtttggaaagaatggttgtcttgcatggttacgaagttgtcaaagaagccttgattgatttgggtgaagaattttcaggtagaggtcattttccattggctgaaagagctaatagaggtttcggtatcgttttctctaatggcaagagatggaaagaaatccgtaggttctctttgatgaccttgagaaattttggtatgggcaagcgttccatcgaagatagagttcaagaagaagctcgttgcttggtagaagaattgagaaagacaaaagcttctccatgtgatccaaccttcattttgggttgtgctccatgtaacgttatctgctcaattatcttccagaagaggttcgattacaaggaccaacagttcttgaacctgatggaaaagttgaacgaaaacatcaggattgtctctaccccatggattcaaatctgtaacaacttcccaaccatcatcgattactttccaggtactcataacaagctgttgaagaacttggctttcatggaatccgatatcttggaaaaggtcaaagaacaccaagagtccatggatattaacaacccaagagatttcatcgactgctttctgatcaaaatggaaaaggaaaagcagaaccagcaatccgagttcactattgaaaacttggttattaccgctgccgatttgttaggtgctggtactgaaactacttctactactttgagatacgccctgctgcttttgttgaaacatccagaagttactgctaaggtccaagaagaaatcgaaagagttgtcggtagaaacagatctccatgtatgcaagatagaggtcacatgccatatactgatgcagttgttcatgaagtccaaaggtacattgatttgatcccaacttctttgccacatgctgttacttgtgatgtcaagtttaggaactacttgatccctaagggtactactattttgacctctttgacttctgtcctgcacgataacaaagagtttccaaatccagaaatgttcgacccaagacacttcttggatgaaggtggtaatttcaagaaatccaactacttcatgccattctctgccggtaaaagaatttgtgttggtgaaggtttggccaggatggaattattcttgttcttgactttcatcctgcaaaacttcaacctgaagtcattgatcgatccaaaggatttggataccactccagttgttaatggttttgcttctgttccaccattctaccaattgtgctttatcccagtttaa

SEQ ID NO: 132 - protein, 497 aa, Homo sapiens [sequence omitted]

SEQ ID NO: 133 - DNA, 1494 bp, Homo sapiens
  source 1..1494: mol_type = genomic DNA
  misc_feature 1..1494: note = 14HC_P450_15
atgggtttagaagctttggttccattggctgttatcgttgctattttcttgttgttggttgacttgatgcatagaaggcaaagatgggctgctagatatccaccaggtccattgccattgccaggtttgggtaatttgttgcatgttgatttccaaaacaccccatactgcttcgatcaattgcgtagaagatttggtgacgtgttctctttacaattggcttggactccagttgttgttttgaatggtttggctgctgttagagaagctttagttactcatggtgaagatactgctgatagaccaccagttccaatcactcaaattttaggttttggtccaagatctcagggtgttttcttggctagatatggtccagcttggagagaacaaagaagattctctgtttctaccttgagaaacttaggtctgggcaagaaatctttggaacaatgggttactgaagaggctgcttgtttgtgtgctgcttttgctaatcattctggtagaccattcagaccaaatggtttgttggataaggctgtctctaacgttattgcttctttgacttgtggtagaaggttcgaatatgatgacccaagattcttgaggttgttggatttggctcaagaaggtttgaaagaagagtctggtttcttgagagaagttttgaacgctgttccagttttgttgcacattccagctttggctggtaaggttttgagatttcaaaaggctttcttgacccaattggatgagttgttgactgaacatagaatgacttgggacccagctcaaccaccaagagatttgactgaagctttcttagctgaaatggaaaaggctaagggtaacccagaatcctcttttaatgacgaaaacttgagaatcgttgtcgccgatttgttttctgctggtatggttactacttctactactttggcttggggtttgttgttgatgatattgcatccagatgttcagagaagagtccagcaagaaattgatgatgttatcggtcaagttagaaggccagaaatgggtgatcaagctcatatgccatataccactgctgttatccatgaagttcaaagattcggtgatattgtcccattgggtgttactcatatgacctccagagatatagaagttcagggttttagaatcccaaagggtactaccttgattaccaacttgtcctctgttttgaaggatgaagctgtttgggaaaagccattcagatttcatccagaacatttcttggatgcccaaggtcattttgttaagccagaagcttttctgccattttcagcaggtagaagggcttgtttgggtgaaccattagctagaatggaactgttcttgttcttcacctctttgctgcaacatttctctttctctgttccaactggtcaacctagaccatctcatcatggtgttttcgctttcttggtttctccatctccatatgaattatgtgccgttccaagataa

SEQ ID NO: 134 - protein, 493 aa, Homo sapiens
  REGION 1..493: note = 14HC_P450_16
MSALGVTVALLVWAAFLLLVSMWRQVHSSWNLPPGPFPLPIIGNLFQLELKNIPKSFTRLAQRFGPVFTLYVGSQRMVVMHGYKAVKEALLDYKDEFSGRGDLPAFHAHRDRGIIFNNGPTWKDIRRFSLTTLRNYGMGKQGNESRIQREAHFLLEALRKTQGQPFDPTFLIGCAPCNVIADILFRKHFDYNDEKFLRLMYLFNENFHLLSTPWLQLYNNFPSFLHYLPGSHRKVIKNVAEVKEYVSERVKEHHQSLDPNCPRDLTDCLLVEMEKEKHSAERLYTMDGITVTVADLFFAGTETTSTTLRYGLLILMKYPEIEEKLHEEIDRVIGPSRIPAIKDRQEMPYMDAVVHEIQRFITLVPSNLPHEATRDTIFRGYLIPKGTVVVPTLDSVLYDNQEFPDPEKFKPEHFLNENGKFKYSDYFKPFSTGKRVCAGEGLARMELFLLLCAILQHFNLKPLVDPKDIDLSPIHIGFGCIPPRYKLCVIPRS

SEQ ID NO: 135 - DNA, 1482 bp, Homo sapiens
  source 1..1482: mol_type = genomic DNA
  misc_feature 1..1482: note = 14HC_P450_16
atgtctgctttgggtgttactgttgctttgttggtttgggctgctttcttgttgttagtttctatgtggcgtcaagttcactcttcttggaatttgccaccaggtccatttccattgccaattattggtaacttgttccagttggagttgaagaacattccaaagtctttcaccagattggctcaaagatttggtccagttttcaccttgtatgtcggttctcaaagaatggttgttatgcatggttacaaggctgtcaaagaagccttgttggattacaaggatgaattttctggtagaggtgatttgccagcttttcatgctcatagagatagaggtatcatcttcaacaatggtccaacctggaaggatatcagaagattctctttgactaccttgaggaattacggtatgggtaagcaaggtaacgaatccagaatccaaagagaagcccatttcttgttagaggctttgagaaagactcaaggtcaaccatttgatcccactttcttgattggttgtgctccatgtaatgttatcgccgatatcttgtttagaaagcacttcgattacaacgacgagaagttcttgagactgatgtacttgttcaacgagaacttccatttgttgtctactccatggttgcagctgtacaacaattttccatctttcttgcattacttgccaggctctcatagaaaggttattaagaatgttgccgaggtcaaagaatacgtgtctgaaagagtaaaagaacaccaccaatctttggacccaaattgtccaagagatttgactgactgcttgttggttgaaatggaaaaggaaaagcactctgctgaaaggttgtacactatggatggtattactgttaccgttgccgatttgttctttgctggtactgaaactacttctaccactttgagatacggcttgttgatcttgatgaagtacccagaaatcgaagaaaagttgcacgaagaaatcgatagagttattggcccatctagaatcccagctattaaggataggcaagaaatgccttatatggatgctgttgttcacgaaatccagaggttcattactttggttccatctaacttgccacatgaagctactagagataccattttcagaggttacttgatcccaaagggtactgttgttgttccaactttagattccgtcctgtacgataatcaagagtttccagatccagaaaagttcaagccagaacacttcttgaacgaaaatggcaagttcaagtactccgattacttcaagccattctctactggtaaaagggtttgtgctggtgaaggtttggctagaatggaattattcttgctgttgtgcgctatcttgcaacactttaatttgaagccattggttgacccaaaggacattgatttgtccccaattcatattggtttcggttgtattccaccaaggtacaagttgtgtgttatcccaagatcttaa

SEQ ID NO: 136 - protein, 503 aa, Homo sapiens
  REGION 1..503: note = 14HC_P450_17
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 137 - DNA, 1512 bp, Homo sapiens
  source 1..1512: mol_type = genomic DNA
  misc_feature 1..1512: note = 14HC_P450_17
atggccttgattccagatttggctatggaaacttggttgttgttggctgtttctttggtcttgttgtacttgtacggtactcattctcatggcttgtttaagaagttgggtattccaggtccaactccattgccattcttgggtaacattttgtcttaccataagggtttctgcatgttcgatatggaatgccataagaagtacggtaaagtttggggtttctacgatggtcaacaacctgttttggctattactgatccagatatgatcaagaccgtcttggtcaaagaatgctactctgttttcactaacagaaggccatttggtccagttggttttatgaagtccgctatttctattgccgaagatgaagaatggaagaggttgagatctttgttgtctccaactttcacttccggcaaattgaaagaaatggttccaattattgcccagtacggtgatgttttggttagaaacttgagaagagaagccgaaactggtaaaccagttactttgaaagatgttttcggtgcctactccatggatgttattacttctacttctttcggcgtcaacatcgactcattgaacaatccacaagatccattcgttgagaacaccaagaagttgttgagattcgatttcttggaccccttcttcttgtccattactgttttcccattcttgatcccaatcttggaagttttgaacatctgcgttttcccaagagaagtcaccaatttcttgagaaagtccgtcaagagaatgaaggaatccagattggaagatacccaaaagcacagagttgatttcttgcaattgatgatcgactcccagaactctaaagaaactgaatctcataaggccttgtccgatttggaattggttgctcaatccatcattttcattttcgctggttacgaaaccacctcctctgttttgtctttcatcatgtatgaattggctacccatcctgatgtccaacaaaagttgcaagaagaaattgacgctgtcttgccaaacaaagctccaccaacttatgatactgtcttgcagatggaatacttggatatggttgttaacgaaaccttgaggttgttcccaattgctatgagattggaaagggtctgtaagaaagatgtcgaaatcaacggtatgttcatcccaaaaggtgttgttgttatgattccatcttacgccttgcatagagatccaaagtattggactgaaccagaaaagttcttgccagaaagattctccaagaagaacaaggataacatcgacccatatatctacactccatttggttctggtcctagaaactgtatcggtatgagatttgctctgatgaacatgaagttggctttgattagagtgttgcagaacttctctttcaaaccctgtaaagaaacccagattccactgaagttgtctttaggtggtttgttgcaacctgaaaagccagttgttttgaaggtcgaatctagagatggtactgtttctggtgcttaa

SEQ ID NO: 138 - protein, 1049 aa, Bacillus megaterium
  REGION 1..1049: note = 14HC_P450_18
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAADEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPRFIDETLAAKGAENIADRGEADASDDFEGTYEEWREHMWSDVAAYFNLDIENSEDNKSTLSLQFVDSAADMPLAKMHGAFSTNVVASKELQQPGSARSTRHIEIELPKEASYQEGDHLGVIPRNYEGIVNRVTARFGLDASQQIRLEAEEEKLAHLPLAKTVSVEELLQYVELQDPATRTQLRAMAAKTVCPPHKVELEALLEKQAYKEQVLAKRLTMLELLEKYPACEMKFSEFIALLPSLRPRYYSISSSPRVDEKQASITVSVVSGPAWSGYGEYKGIASNYLAELQEGDTITCFISTPQSEFTLPKDPETPLIMVGPGTGVAPFRGFIQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 139 - DNA, 3150 bp, Bacillus megaterium
  source 1..3150: mol_type = genomic DNA
  misc_feature 1..3150: note = 14HC_P450_18
atgactataaaagagatgccacaacctaagaccttcggcgagctaaagaacctgcctctattgaatacggacaagccagtgcaggcccttatgaaaattgcggatgagctgggtgagatcttcaaatttgaagcgccagggcgtgtgactagatatttatccagccaaaggctgataaaagaggcttgcgacgagtctcgtttcgataagaacctgagccaggcggccaaatttgccagggatttcgcgggagacgggttggtaacgtcttggactcacgagaaaaattggaaaaaggcgcataatatattgctaccttccttctcccaacaagcgatgaagggttaccatgcaatgatggttgatattgcagtgcagttagtccaaaagtgggagaggctgaatgctgatgagcacattgaagtctctgaggacatgacgcgtctaacattggatacaattggcttatgtggatttaactatagattcaactctttctatagagaccagcctcatcctttcattatcagcatggtccgtgccgcagacgaggtcatgaataaattgcagcgtgctaacccagatgatcccgcatacgatgagaacaaaaggcagttccaggaggatatcaaagtaatgaatgatcttgtcgacaaaatcatagcggatcgtaaggcaaggggagagcagtctgacgacttgctgacgcaaatgctgaacggaaaagatcctgaaacaggtgaacccctagacgacgggaacatccgttaccagataataacttttttaattgccgggcacgagacaaccagtggactgctgtcctttgccctgtatttcttagtcaagaaccctcatgtgttacaaaaagtcgccgaggaagcagctagagtcctagtagaccccgtgccaagctataaacaggttaagcaacttaaatacgttgggatggtcctgaacgaggcattaaggttatggcccaccgcacctgcgttttcactttatgcgaaagaagatacggttctgggtggcgagtaccctcttgaaaagggagacgaagttatggttttgataccgcagcttcatagggacaagactgtctggggggatgatgtcgaagaatttcgtcctgagaggttcgagaatcctagtgctatcccgcaacacgcgttcaaaccctttggtaacggacaaagggcctgtatcggccagcaattcgccctacacgaggcgacacttgttttaggcatgatgctaaagcattttgactttgaggatcacacgaactacgagctggatatcaaagagacacttaccttgaaacctaagggtttcgtcgtcaaagctaaaagtaagaagatccctttgggaggcatcccgtcaccgtccacggaacaatctgcgaagaaagttaggaaaaaagccgagaatgcacataacaccccgttattagttctttacgggtctaacatgggaacggccgagggaacggcccgtgacttggcagacatagctatgtcaaaagggtttgccccgcaggtcgcgactcttgatagccatgcgggcaatctaccgagggagggcgccgttttgatcgtaactgcatcctacaatggtcacccccccgacaacgccaagcaatttgtagactggcttgaccaagcgtccgctgatgaggtaaagggcgtgcgttatagtgttttcgggtgtggcgataagaattgggctactacataccaaaaggttccacgtttcatagatgaaactctggccgcaaagggcgcggaaaacatcgccgaccgtggagaagcagatgctagtgatgacttcgaaggcacctacgaagagtggcgtgaacacatgtggtccgatgtggctgcgtattttaacttagatatcgagaattctgaggacaacaaaagtacgctatccctacagttcgtagattctgccgcggacatgcctttggctaaaatgcatggagcattctcaacaaatgtggtggcctctaaagagctacagcagcccggttctgcccgttccactcgtcatatcgaaatcgagttaccaaaggaagcttcctatcaagagggagatcacctgggggttattccacgtaattacgagggcatcgttaacagggtaactgcccgttttgggttagatgcctcacaacagatacgtctggaggccgaggaagaaaaactagctcacttgccactggcgaagaccgtcagcgtcgaggagctgctgcaatacgttgaactgcaagacccagctactaggacgcaattgcgtgctatggcagctaaaacggtatgtccacctcataaagtggaacttgaggctctgctggaaaagcaggcgtataaagaacaagtgctggcaaaaaggctaaccatgctagaactgcttgagaagtacccggcgtgcgaaatgaaattctccgagttcatagccctgttgccatcacttcgtcctaggtattactcaatatcctcatctccaagggttgatgagaagcaagcgagcataactgtttccgtcgtcagcggtccagcgtggagcggatacggtgagtacaaaggaattgcctctaactacctggcagagttacaggagggggacactattacgtgcttcatctctactccgcaatcagagttcactctgcccaaagatcctgaaactcccctaataatggtaggcccaggcaccggagtagcgccattcagaggttttattcaggctagaaaacagcttaaagaacagggccaatctttaggtgaagcccatctttactttggttgtcgttcccctcacgaggactatttataccaggaggaacttgaaaacgcacagagtgaaggaatcataacactacataccgcattctccaggatgccgaatcaaccaaagacttacgttcaacacgtcatggagcaagatggaaaaaagctgatcgagttacttgatcaaggtgcgcacttttatatctgcggggatggttctcagatggctcctgcggtagaggccaccctaatgaaaagctatgcagacgtacaccaggtatccgaggcggacgctaggctgtggctgcagcaattagaggaaaagggtcgttacgcgaaagatgtgtgggcgggatga

SEQ ID NO: 140 - protein, 1049 aa, Bacillus megaterium
  REGION 1..1049: note = 14HC_P450_19
MTIKEMPQPKTFGELKNLPLLNTDKPVQALMKIADELGEIFKFEAPGRVTRYLSSQRLIKEACDESRFDKNLSQGLKFVRDFAGDGLATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKQQRANPDDPAYDENKRQFQEDIKVMNDLVDKIIADRKASGEQSDDLLTHMLNGKDPETGEPLDDENIRYQIITFLIAGHVTTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 141 - DNA, 3150 bp, Bacillus megaterium
  source 1..3150: mol_type = genomic DNA
  misc_feature 1..3150: note = 14HC_P450_19
atgactataaaagagatgccacaacctaaaacctttggcgagttaaagaacctgccactactaaatacggataagccggtccaggctctgatgaaaattgcggatgagctgggggagatttttaagtttgaagcaccgggaagggttactcgttatctttcctctcagaggttaatcaaagaggcatgtgatgaatcaagattcgacaagaacctgtctcagggactaaagtttgtgcgtgactttgccggtgatggccttgccacgtcctggacccatgagaagaactggaagaaagctcacaacatcctacttccctcattctctcagcaagcgatgaagggctaccacgctatgatggtcgacatcgctgtccagctggtacaaaaatgggagagacttaatgcggatgaacatatagaagtgcctgaagatatgactagattgactttagacaccataggcctgtgcgggtttaactacaggttcaacagcttttaccgtgaccagcctcatccgttcattacatctatggtacgtgcccttgacgaagctatgaataagcagcaaagagcaaatcccgacgaccctgcctatgatgagaataagaggcagttccaggaggacattaaggttatgaatgatttagttgataagatcatcgcggacaggaaagcgtcaggtgaacaatctgatgatctactgacacatatgttaaatggaaaagatcccgagactggagagcctttagacgacgaaaacataagatatcaaattatcaccttcctgattgctggacacgtaacaacctctgggttactatcatttgccttatactttctagtcaagaaccctcatgtccttcaaaaggctgcggaagaagcagcgagggtgctagtcgacccggtcccctcttacaagcaggtcaagcaactgaaatatgtcggaatggtcctgaatgaggcgctaaggctatggccgacggctcctgctttcagtctatacgccaaggaagataccgtcctgggaggtgaatatcccctggaaaaaggtgatgaactaatggtattaattccacagctgcacagggacaagacgatttggggagacgatgtagaggaattcagacccgaaagatttgagaacccctctgcaattccccaacatgcgtttaagccgtttggcaatggacagagagcctgcatcggacagcagtttgctctacacgaagcgacgctggtgctagggatgatgttaaagcattttgatttcgaagaccacacaaactatgagctggatataaaagagacattgaccttgaagccagaagggtttgtcgtgaaagctaagtctaaaaaaatacctctgggagggattccatccccgagtaccgagcaaagtgccaaaaaggtaagaaagaaagcggaaaacgcccacaacaccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 142 - protein, 1049 aa, Bacillus megaterium
  REGION 1..1049: note = 14HC_P450_20
MTIKEMPQPKTFGELKNLPLLNTDKPVQALMKIADELGEIFKFEAPGLVTRYLSSQRLIKEACDESRFDKNLSQGLKFVRDFAGDGLATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKQQRANPDDPAYDENKRQFQEDIKVMNDLVDKIIADRKASGEQSDDLLTHMLNGKDPETGEPLDDENIRYQIITFLIAGHVTTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 143 - DNA, 3150 bp, Bacillus megaterium
  source 1..3150: mol_type = genomic DNA
  misc_feature 1..3150: note = 14HC_P450_20
atgacgattaaggagatgccccagccaaaaacattcggcgagctgaagaacttgccgctgttaaataccgacaagccggtccaggcattgatgaaaatagcggacgaattaggagaaattttcaaatttgaagcgccgggcctagtgacaagatacttatcatcacagagactaatcaaggaagcgtgtgatgagagccgtttcgataagaacctaagccagggattgaaatttgtgcgtgatttcgcgggtgatggtttggctacctcttggacgcacgaaaagaactggaaaaaggctcacaacatattgctgccctccttcagccaacaggcgatgaagggctatcatgctatgatggtagacattgcggttcagctggtacagaagtgggagaggctgaacgcagatgagcatattgaggtccctgaagatatgacaaggctgaccctagacactataggattatgcggctttaattaccgttttaattcattctacagggatcaaccacatccattcatcacatcaatggtacgtgcattagacgaagcaatgaacaagcagcagagggcgaacccagatgaccctgcatatgacgagaacaagaggcaatttcaggaggacatcaaggtaatgaatgaccttgttgacaagatcatcgccgacagaaaggcatccggggaacagtctgacgatctgctaacacacatgcttaacggcaaagatcccgagactggggagcctcttgacgatgagaatatccgttaccaaatcattacgttcttaatcgcggggcacgttaccacatctggcttactttcattcgccttatatttcttagtaaagaacccacacgtcctgcaaaaggccgcagaagaggctgcgcgtgtccttgtggacccggttccaagttataaacaggtgaaacaactaaagtatgtgggtatggtactaaatgaggccctgcgtctttggcctaccgcccctgcctttagtctatatgcaaaggaggacactgtattaggtggggaatatcccctggagaagggcgatgagctaatggtgctgattcctcaactacatagggataaaactatctggggagatgacgtagaggaattcagacctgaaaggtttgaaaacccgagtgcgatacctcaacatgctttcaaaccattcggcaatggtcagagggcatgtataggacaacaatttgcactacatgaagcaacgctagtgctgggtatgatgctaaagcacttcgattttgaggatcacactaactatgaactagacataaaagagactcttacccttaaaccggaagggttcgtagtgaaagcgaagagtaaaaaaataccccttggaggtataccctcccctagcaccgaacagtcagctaagaaagtgcgtaaaaaggcggagaacgctcacaataccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 144 - protein, 1049 aa, Bacillus megaterium
  REGION 1..1049: note = 14HC_P450_21
MTIKEMPQPKTFGELKNLPLLNTDKPVQALMKIADELGEIFKFEAPGLVTRYLSSQRLIKEACDESRFDKNLSQGLKFVRDIAGDGIATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKQQRANPDDPAYDENKRQFQEDIKVMNDLVDKIIADRKASGEQSDDLLTHMLNGKDPETGEPLDDENIRYQIITFLIAGHVTTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 145 - DNA, 3150 bp, Bacillus megaterium
  source 1..3150: mol_type = genomic DNA
  misc_feature 1..3150: note = 14HC_P450_21
atgactattaaagagatgccgcaacctaaaacttttggggaactaaaaaacttaccacttctaaacacagacaaacctgttcaagcgttgatgaagatcgcagatgagctgggagaaattttcaagttcgaggcaccggggctggtaacgcgttaccttagtagccagcgtctaattaaagaagcgtgtgatgaaagtaggttcgacaagaatttatcacaggggcttaagttcgtcagggatattgccggtgatgggatcgctacaagctggacacatgagaaaaattggaagaaggcccataatatccttctacccagtttcagccaacaggctatgaaggggtatcatgcaatgatggtggatattgctgttcagttagtccagaagtgggaacgtcttaatgcggatgagcacatagaagtgcctgaagatatgacaagactgacgcttgacaccataggcctgtgtgggttcaactatagattcaatagtttttatagagaccaaccgcatccgtttatcacgtctatggtgagagcactggatgaggctatgaacaagcaacaaagggcaaatcccgatgatccagcttacgacgagaataagcgtcagtttcaagaagacatcaaggtaatgaatgatctagtggacaaaattatagccgatagaaaagcctctggtgaacagagcgatgatctgctaacacatatgctgaacggcaaagatccagagactggagagcctctggatgatgaaaatataagataccaaataattactttcttaatcgcgggccatgttactacgtcaggactgttaagttttgcattgtactttctagttaagaatccccacgttctgcaaaaagcggcggaggaggccgccagagtcttagttgacccagtgccctcctacaaacaggtaaaacagcttaagtatgtgggtatggtgttaaatgaggcacttcgtttgtggcccactgccccagcgtttagtctatacgcaaaagaagacaccgtcttaggaggtgaatatccgcttgagaagggagatgaattgatggtcctaattccccaattacatagagacaaaaccatctggggagacgacgttgaagagttcagaccagagaggttcgaaaacccttctgccataccacagcacgctttcaaacctttcggaaatggtcagagggcctgtataggacaacagtttgcgcttcatgaagcaacgcttgtacttggaatgatgctgaagcactttgactttgaggaccacacgaattacgaattagacatcaaggagacattgacacttaagcctgaaggcttcgttgtaaaggcgaagtccaaaaagatcccacttggtggtataccatcaccatccaccgaacaaagcgcgaagaaggttaggaaaaaagctgaaaatgctcataacaccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 146 - protein, 1049 aa, Bacillus megaterium
  REGION 1..1049: note = 14HC_P450_22
MTIKEMPQPKTFGELKNLPLLNTDKPVQALMKIADELGEIFKFEAPGLVTRYLSSQRLIKEACDGSRFDKNLSQGLKFVRDIAGDGLATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKQQRANPDDPAYDENKRQFQEDIKVMNDLVDKIIADRKASGEQSDDLLTHMLNGKDPETGEPLDDENIRYQIITFLIAGHVTTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 147 - DNA, 3150 bp, Bacillus megaterium
  source 1..3150: mol_type = genomic DNA
  misc_feature 1..3150: note = 14HC_P450_22
atgacgataaaggagatgccccaacccaagacctttggagagctaaagaatttgccccttctgaacactgataagccggtccaggcactaatgaagattgcagacgaattgggggaaatatttaagtttgaagctccgggtttggttacaaggtaccttagcagccaacgtctaatcaaggaagcgtgcgatggaagtcgtttcgacaaaaacctaagccaaggcttaaagttcgtgagagacatagccggggacggcttagcaaccagttggacccacgaaaagaattggaagaaggcacataatatactgcttccgagcttttcacaacaggctatgaagggatatcatgcgatgatggtcgatatagcggtacaacttgtacaaaaatgggagcgtctaaatgctgacgagcatatcgaggtgcctgaagacatgactagactgacgcttgataccattgggctgtgcggctttaactatagattcaacagcttttatcgtgaccaaccacacccctttattaccagcatggttagagcgctagacgaggccatgaataagcagcaaagagcaaatcccgacgatccagcatatgacgaaaataagcgtcagtttcaagaagacataaaggtaatgaatgacctggtcgataaaataattgctgatagaaaggcctcaggggagcagtccgacgatttacttacccacatgctgaacggtaaggatcctgaaaccggggaaccactagacgacgaaaacataaggtaccaaattattacgttcttgatcgccggacacgttaccacttctggcttactatcctttgcgttgtactttcttgttaagaatccacacgttctacagaaagccgccgaggaggcggcacgtgtcctagttgaccccgtaccgtcctataaacaggttaaacaactgaaatacgtcggcatggtactgaacgaagctctaaggctatggcccaccgccccagccttctccctttatgcgaaggaggacaccgtcttaggaggagagtacccattggaaaaaggcgacgaactaatggtgttaattccccagcttcacagagataagacgatatggggagatgacgtggaggaattccgtccggaaaggttcgaaaatccatccgcaataccacagcacgccttcaagccttttgggaatggacaaagggcgtgtatagggcaacaattcgctttacatgaagcgacgctagtgctaggaatgatgctgaaacacttcgatttcgaggatcacaccaattatgagctggacataaaagaaaccctgactctaaaaccggagggattcgtggtgaaagccaagtccaagaaaattcctcttggtggtataccgtcaccctctacggagcaatctgcgaaaaaagtacgtaaaaaagcggagaacgcgcataacaccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 148 - protein, 1049 aa, Bacillus megaterium
  REGION 1..1049: note = 14HC_P450_23
MTIKEMPQPKTFGELKNLPLLNTDKPVQALMKIADELGEIFKFEAPGLVTRYLSSQRLIKEACDESRFDKNLSQGLKFVRDIAGDGLVTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKQQRANPDDPAYDENKRQFQEDIKVMNDLVDKIIADRKASGEQSDDLLTHMLNGKDPETGEPLDDENIRYQIITFLIAGHVTTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 149 - DNA, 3150 bp, Bacillus megaterium
  source 1..3150: mol_type = genomic DNA
  misc_feature 1..3150: note = 14HC_P450_23
atgacaattaaggaaatgccacaaccgaaaacttttggtgagctgaagaatttacctctgcttaacacggacaaacctgtgcaagccttaatgaaaattgcggacgagttaggagaaattttcaagtttgaggcacctggattggtgactcgttatctaagcagtcaaaggctaattaaggaagcttgcgacgagagcagatttgataaaaacctgtcacaagggttgaaatttgtccgtgatatcgcaggcgatggtcttgtcactagttggacgcatgaaaagaactggaagaaggcacataatattctattaccgtctttcagccagcaggcgatgaaagggtatcacgcaatgatggtcgacatcgcagtccagcttgttcagaaatgggagaggttgaacgccgacgagcatatagaggtacctgaggacatgaccagattgacattggatacaatagggctatgtggctttaattatcgttttaactctttctatagagaccaaccccatccattcatcacctccatggtaagagctctagatgaagcgatgaacaaacaacaaagagctaatcccgatgaccctgcctacgacgagaacaaacgtcagtttcaagaggacataaaagtcatgaatgatcttgtggacaagattattgcagataggaaagcatccggagaacagtctgacgaccttttaacccatatgcttaacggcaaggaccccgaaacgggtgaacctcttgacgacgagaatataaggtatcagataattaccttcttaatagctggtcacgtcacgacctccggcttgttgtcctttgcgctatattttctagttaaaaacccgcatgttttacagaaggcagctgaggaagctgcgagggtgctagttgatcccgtcccgtcatataagcaggtgaaacagctgaagtatgtgggaatggtacttaacgaggctctacgtttatggccgaccgcgcccgcgttctctttatacgcgaaggaagataccgtgctaggcggcgaatatcccttagagaagggtgacgaacttatggttctgatacctcagttacatcgtgataaaaccatctggggagacgacgtcgaagaattcaggcccgagaggtttgagaacccgtctgcaatcccccaacatgcgttcaaaccgttcggaaacggccaaagagcgtgtattgggcagcaattcgccttgcacgaggcaactttggtcttgggcatgatgcttaagcacttcgactttgaagatcatacgaactatgagttagacataaaggagacgttgaccctaaaacctgaaggctttgtagttaaagccaagtccaagaagatcccgctgggaggaatcccgtccccgtccaccgaacaatccgcaaagaaagtgagaaaaaaagctgaaaatgcacacaataccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 150 - protein, 1049 aa, Bacillus megaterium
  REGION 1..1049: note = 14HC_P450_24
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISAVRAADEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 151 - DNA, 3150 bp, Bacillus megaterium
  source 1..3150: mol_type = genomic DNA
  misc_feature 1..3150: note = 14HC_P450_24
atgacgataaaagaaatgccccagccaaagacatttggggagcttaagaatctaccattgctaaacaccgataaaccggtgcaagcacttatgaaaattgctgacgaactaggtgaaatattcaagtttgaagctcctggcagagtaacaaggtacttatcatctcaaagattaatcaaggaggcctgcgacgagtctagattcgacaaaaatctatctcaggcagccaaatttgctagagatttcgccggggatggtttggtgacttcctggacacatgagaagaattggaagaaagctcataacatattactaccatccttcagtcaacaagcgatgaagggatatcatgccatgatggttgatatcgctgttcagttggtccaaaaatgggaacgtctgaacgcagatgaacatatcgaggtctctgaggatatgactaggttaacactggacactatcggtttgtgtggatttaactatcgttttaatagcttctatagagaccaaccccacccattcatcatatctgctgtacgtgcggcagatgaggtcatgaataagctgcagagggctaatccggacgacccagcgtacgacgaaaacaagagacagtttcaagaagacataaaggtgatgaatgaccttgtagacaagatcatagctgatagaaaggcccgtggagaacaatccgatgatctgcttacgcagatgttgaatggcaaggatcccgagacaggagagccactggatgatgggaacattagataccagataattacattcctaatcgcggggcacgagaccacgtctggcctactatcattcgctctttattttttggtgaaaaatccacatgtcttacagaaagtcgcggaggaggcggccagagttctagttgaccccgttccaagctacaagcaagtaaagcaacttaaatatgttggaatggtccttaacgaagcattgagactatggccgaccgcccccgcattctcattatacgcaaaggaagataccgttctggggggggaataccctctagagaaaggtgacgaagtaatggtcctaatcccccagcttcatcgtgacaaaacagtatggggcgacgacgtcgaagaattcaggcctgaaagattcgagaatccgtcagcaatacctcagcacgctttcaagccctttgggaatggtcagagggcgtgtatagggcaacagttcgcattacatgaagcgacactggtgctgggaatgatgcttaagcatttcgattttgaggaccacacgaattacgaactagacattaaggaaacactgaccctaaaaccaaagggctttgtcgtgaaggcaaaaagcaagaaaatcccgttgggtggcattccctctcctagcacagagcaaagtgccaaaaaggttagaaagaaagcggagaatgcccataacaccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 152 - protein, 1049 aa, Bacillus megaterium
  REGION 1..1049: note = 14HC_P450_25
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISAVRAADEVMNKLQRANPDDPAYDENKRQFQEDIKVMNDLVDKIIADRKARGEQSDDLLTQMLNGKDPETGEPLDDGNIRYQIIAFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 153 - DNA, 3150 bp, Bacillus megaterium
  source 1..3150: mol_type = genomic DNA
  misc_feature 1..3150: note = 14HC_P450_25
atgactattaaagaaatgccccaacccaagacgtttggcgagttaaaaaacctgccgctattaaatacggataagccggtccaggcactaatgaagatcgctgatgagctgggcgaaatcttcaagttcgaggccccaggaagagtgactaggtatcttagctcacagagactgataaaagaagcgtgtgacgagtctagattcgacaagaatttatcccaagcggccaagtttgcgcgtgattttgcgggtgatgggctggtgacgtcctggactcatgagaagaactggaagaaagcccataacattttgctgccaagtttctctcagcaagcgatgaagggttaccatgcgatgatggttgatatcgctgttcaattggtacaaaaatgggagagattaaacgcggatgagcatatcgaagtcagcgaagacatgactagacttacgcttgacactataggactttgcggttttaactatagatttaattccttttaccgtgatcaaccccatcccttcataatctcagcggtaagagctgcggacgaggtcatgaataagctgcagcgtgcgaaccctgacgatcctgcgtacgatgagaataaaaggcaatttcaagaagacatcaaagttatgaacgacttagttgacaagattattgcagatagaaaagcgcgtggagaacagtccgacgatcttttaacgcagatgttaaatggaaaagacccagaaacgggagagcctctagacgacggtaatattcgttatcagattatagctttcctgatagcagggcacgagaccacttcaggcttattaagcttcgccctgtactttctagtcaagaacccccacgtcctgcagaaggtagctgaagaggctgcaagggtccttgtagatcctgttccttcttataaacaggtcaaacaattaaagtacgttgggatggttcttaacgaagcgttacgtttatggcccaccgctcctgctttttctttgtatgcgaaagaagacaccgttcttggaggtgaatatcctctagagaaaggagatgaggttatggttttaattccgcagcttcaccgtgataaaacggtttggggagatgacgtcgaggaattccgtcccgaaaggttcgagaatccgagcgcgatcccccaacacgcattcaagcccttcgggaatgggcaacgtgcctgtataggtcagcaattcgccctacacgaggccactcttgttctgggcatgatgttgaaacattttgactttgaagatcacactaattatgagttggatatcaaggaaacgttgacactgaaaccaaaaggattcgtagttaaagccaaaagcaaaaaaatacccttagggggtataccttctccctcaactgagcagtctgcgaaaaaggtaagaaagaaagcagagaacgcacataacaccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 154 - protein, 1049 aa, Bacillus megaterium
  REGION 1..1049: note = 14HC_P450_26
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAADEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 155 - DNA, 3150 bp, Bacillus megaterium
  source 1..3150: mol_type = genomic DNA
  misc_feature 1..3150: note = 14HC_P450_26
atgacaataaaggaaatgccgcaacctaaaactttcggcgaattaaagaatctaccactacttaacacagacaaaccggtccaagctttgatgaagatcgcagacgagctaggcgaaatctttaaatttgaggcaccaggaagagtaacaagatacttgagttctcaaagactgatcaaggaagcctgtgatgaatccaggtttgataaaaatctatctcaagctgccaaattcgctcgtgattttgcgggcgatggactagttacaagttggacccacgagaaaaactggaagaaggcgcacaatatcttgttacccagtttctcccaacaggccatgaagggttatcacgccatgatggttgatatcgcggtacagctggttcaaaaatgggaacgtttgaatgcagacgagcatatagaggtgagtgaagacatgacgcgtcttactctggataccatcgggttgtgtgggttcaactacaggtttaatagtttttacagggaccagccccatccattcataattagtatggtgagggccgctgacgaagtaatgaacaaattacaacgtgccaatcccgacgaccctgcctatgacgagaataaacgtcagttccaagaggatataaaagtgatgaatgatcttgttgataagattatagcagatagaaaggcgagaggagaacaaagcgatgatttgctgactcagatgctaaacggaaaggaccccgagacgggagagcccttggacgatggtaatattagatatcagatcataaccttcctgatcgccggacacgaaacaacgagcggacttcttagcttcgccctatactttctagttaagaaccctcacgtgctgcagaaggttgcggaggaggcagccagagtgttggtcgatcctgtaccgtcatacaaacaagtcaagcagcttaaatacgttggaatggtgctaaacgaggcgttgaggctttggcctacggcacctgccttctcattgtacgcgaaagaggacacggtactaggcggagaatatcctttggagaagggcgatgaagtcatggtcttaattccacaattacacagagacaaaacggtctggggggatgacgttgaagagtttagaccagagaggttcgaaaatccatccgcgatacctcagcatgccttcaaaccgtttggcaatggacagagagcatgcattggacaacaattcgcccttcatgaggctacacttgtcctggggatgatgttgaagcatttcgactttgaggaccatacaaattacgagctagacattaaggaaacgctaacattaaagccgaagggcttcgtcgtaaaagctaagagtaagaagataccgttaggaggtatcccttcccctagtacagaacagagtgcaaagaaggttcgtaaaaaggcggagaacgcacataataccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 156 - protein, 1049 aa, Bacillus megaterium
  REGION 1..1049: note = 14HC_P450_27
MTIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETATLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 157 - DNA, 3150 bp, Bacillus megaterium
  source 1..3150: mol_type = genomic DNA
  misc_feature 1..3150: note = 14HC_P450_27
atgactataaaggagatgccacaaccaaaaaccttcggcgaacttaaaaatttgccattattgaacactgataaaccggttcaggcgctaatgaaaatagcagacgaattaggggagatctttaaatttgaggccccagggagggtcaccagatatctttcctctcaacgtttgataaaggaagcctgtgacgagtccagatttgataaaaacttatcacaagctctgaagttcgccagagacttcgctggtgatggactagttacatcatggactcacgagaagaactggaaaaaagcacataacattctgcttcccagtttctcacaacaggcaatgaaaggttaccatgcgatgatggtggacattgcggtgcagcttgtacaaaaatgggaaaggttaaatgctgatgaacatatcgaggtgagtgaagacatgaccaggcttactctagacacgatcggattatgcggtttcaactacagatttaattctttctacagagaccagccacaccctttcataatctctatggtccgtgcattagatgaagtgatgaataagttgcaaagggctaatcccgacgacccggcctacgacgaaaataagaggcaattccaagaagatattaaagtgatgaatgatcttgtggacaaaattattgcagatcgtaaggcgaggggggagcagtctgatgacttactaactcagatgttgaatggcaaggatcccgagaccggagaaccactggacgatggcaacatccgttatcagattataactttcttaatcgctggccacgagaccacatctggacttctgtcatttgcactatacttcctagttaagaatccgcacgtgctgcagaaggtggcggaagaggctgctcgtgtcctagtcgaccccgtacccagctataaacaggtaaaacagcttaagtatgttgggatggtcctgaatgaggcgctgagactatggccaactgcgccagcctttagcctttatgccaaagaggataccgtcttaggaggtgagtatccactggagaaaggagatgaagttatggtgcttatccctcagttgcatcgtgataagaccgtgtggggtgatgatgtggaagaatttaggcccgagagatttgagaatccttccgcgattccccagcatgctttcaagccctttggcaacggacagagagcttgcattgggcaacagtttgctttgcatgaggcaacgctagttttggggatgatgctaaagcatttcgatttcgaagaccatactaactatgaacttgatatcaaggagacagctaccctaaaacccaagggtttcgtggttaaggcgaagtctaaaaaaataccactaggggggatcccatcaccgtcaactgagcagtccgccaaaaaagtccgtaagaaagccgaaaatgcgcataataccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 158 - protein, 1049 aa, Bacillus megaterium
  REGION 1..1049: note = 14HC_P450_28
MTIKEMPQPKTFGELKNLPLLNTDKPVQALMKIADELGEIFKFEAPGLVTRYLSSQRLIKEACDESRFDKNLSQALKFVRDFAGDGIVTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKQQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLTEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 159 - DNA, 3150 bp, Bacillus megaterium
  source 1..3150: mol_type = genomic DNA
  misc_feature 1..3150: note = 14HC_P450_28
atgacaatcaaagaaatgccccagccgaaaaccttcggtgagctaaaaaatttgcccttattaaacacagataagccggtacaagctcttatgaaaatagccgatgaactgggggaaatttttaagtttgaagctcctggactagtaactagatatttgtcttctcaaagactaatcaaagaggcatgcgatgagtccagattcgataaaaatttaagtcaggctcttaaattcgttagagactttgccggtgacggaattgttacaagctggacacatgaaaaaaactggaaaaaggcccataacattttactgccgtccttttctcaacaggcaatgaaagggtaccacgcaatgatggtcgatattgccgttcagttagtacagaaatgggagagactaaatgcggatgaacatatagaggtgccagaagatatgacgaggttaacattggacaccattggtctgtgcggatttaattaccgtttcaacagtttctaccgtgatcagccccatccatttataaccagcatggtcagagcgcttgatgaagctatgaataaacaacaaagagccaatcctgacgacccagcatatgatgagaacaaaagacagttccaggaagatataaaagtgatgaatgatttggtagacaagatcatagccgacagaaaggcttcaggcgaacagagtgacgatttattaactcatatgctaaatggtaaggacccggaaacaggcgagcctcttgatgatgagaacataagatatcaaataatcacatttttgatcgcgggccatgagacgacgagtggcttgttaagtttcgcgctttattttttagtaaaaaaccctcatgtgcttcagaaagcggctgaagaggcggctcgtgttttggtagacccagtgcctagctacaaacaagttaagcaacttaaatatgtcgggatggtattaaccgaggcgctgcgtttgtggccaacagcccctgcgttctccctatacgcaaaggaggacacagtgctaggcggcgagtatccattagaaaagggagatgagctaatggttttgatccctcaacttcaccgtgacaaaacgatttggggcgatgatgtggaggagttcaggccagaaaggttcgagaacccaagtgctatcccccagcacgcatttaagccctttgggaacggccagcgtgcctgtattggccaacagttcgcactgcacgaggcgacgcttgtccttgggatgatgctaaaacattttgattttgaggaccatacaaattacgagttagatataaaggaaactctgactctaaaaccggaaggttttgtcgttaaagccaaatcaaagaaaatacctcttggtggaataccgtccccgagcactgagcagtccgcgaaaaaagtacgtaagaaggcggaaaatgctcacaataccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 160 - protein, 1049 aa, Bacillus megaterium
  REGION 1..1049: note = 14HC_P450_29
MTIKEMPQPKTFGELKNLPLLNTDKPVQALMKIADELGEIFKFEAPGLVTRYLSSQRLIKEACDESRFDKNLSQALKFVRDIAGDGLVTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKQQRANPDDPAYDENKRQFQEDIKVMNDLVDKIIADRKASGEQSDDLLTHMLNGKDPETGEPLDDENIRYQIITFLIAGHVTTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLSMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 161 - DNA, 3150 bp, Bacillus megaterium
  source 1..3150: mol_type = genomic DNA
  misc_feature 1..3150: note = 14HC_P450_29
atgacaattaaagagatgccgcaaccgaaaacttttggtgaactaaaaaacttgcctttgctaaacacggataaacctgtgcaggccttaatgaaaattgcagatgagctaggtgaaatttttaaattcgaagcccccggcttagtgacgaggtatctatcatcccagaggctgattaaggaggcttgcgacgaatccagattcgataagaacctaagccaagcgctgaagttcgtaagggacatcgctggcgacgggctggtgacttcctggacccatgaaaaaaattggaaaaaggctcataacatcttgttaccatcattttcccaacaggcgatgaagggttatcacgctatgatggtagatatcgccgtccagttagtccaaaagtgggaacgtcttaacgccgacgagcatatcgaggtacctgaggacatgacaaggctaacgttagacacgatcggattgtgtggtttcaattaccgtttcaactcattctatagggaccaacctcatccctttataacgagcatggttcgtgccctggatgaagcaatgaacaaacaacagagggccaatccggacgacccggcatatgatgagaacaagagacagtttcaagaggatataaaagttatgaatgaccttgtagacaagatcatcgcggatcgtaaggcgtctggggaacagtctgacgaccttttaacgcatatgcttaatggaaaggacccggagacaggagaaccattggatgatgagaacattaggtatcagataatcacttttcttatagcaggccatgtcacgacgtccgggctgttatcctttgctctatattttctagtgaagaaccctcacgtattgcagaaggcagccgaggaagctgctagagtcctagttgatccggtccctagttataaacaagttaaacagttgaaatatgttggtatggtgttaaatgaggcactgagactatggcctacggcaccagctttcagcttgtatgccaaggaagatacggtcttagggggagaatatccactagagaagggcgatgaattgatggtgttgattccgcaattgcacagagacaaaacgatttggggggacgatgttgaggaattccgtcctgaaagatttgagaacccatcagccatcccccagcacgcattcaagccctttgggaacgggcagagggcatgtatcggccaacagttcgcactgcatgaggcaaccctggtactatcaatgatgttaaaacatttcgacttcgaagaccacaccaattatgagttagacatcaaggagactctgaccctgaaaccggaaggattcgtggtaaaggccaagtcaaagaagattccactaggtggtattccgagcccgtccactgagcaaagtgctaagaaggttcgtaaaaaagcggaaaatgcacacaataccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 162 - protein, 1049 aa, Bacillus megaterium
  REGION 1..1049: note = 14HC_P450_30
MTIKEMPQPKTFGELKNLPLLNTDKPVQALMKIADELGEIFKFEAPGLVTRYLSSQRLIKEACDGSRFDKNLSQALKFVRDIAGDGLVTSWTHEKNWKKAHNILLPSFSQQAMKGYHAMMVDIAVQLVQKWERLNADEHIEVPGDMTRLTLDTIGLCGFNYRFNSFYRDQPHPFITSMVRALDEAMNKQQRANPDDPAYDENKRQFQEDIKVMNDLVDKIIADRKASGEQSDDLLTHMLNGKDPETGEPLDDENIRYQIITFLIAGHVTTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLSMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 163 - DNA, 3150 bp, Bacillus megaterium
  source 1..3150: mol_type = genomic DNA
  misc_feature 1..3150: note = 14HC_P450_30
atgacaataaaggagatgccccagcccaaaacattcggggaattaaaaaatctaccacttcttaacaccgacaaacctgttcaagcgctgatgaaaattgcggacgaacttggggagattttcaagttcgaagcaccgggtcttgtgacccgttatttatcttcccagaggcttattaaggaggcttgtgacggctcaaggtttgataaaaatttgagccaagctctgaaatttgtcagagatatagccggtgacggcctggtcacaagctggactcacgaaaaaaattggaagaaggctcataatatactattgccctcatttagccagcaggctatgaaaggatatcacgcgatgatggtcgatatcgcagtgcaactagtgcagaagtgggaacgtttgaacgcggatgaacatattgaggtacctggggatatgaccaggctaactcttgacacgatagggttgtgtggttttaactataggttcaattcattttatagggatcaaccacacccgttcataaccagcatggtgcgtgcgttggatgaagcgatgaacaaacaacagagggcgaacccagatgatccagcttacgatgaaaataaaagacaatttcaggaagatattaaagtaatgaacgatttggttgataaaataatagcggataggaaggctagcggcgagcaatccgacgatcttcttacccacatgttaaacggcaaagatccggagacgggggagccattggatgatgaaaacataagataccagattatcacatttcttatcgcgggacatgtcactacatccgggcttctaagctttgcactatacttcctagtgaagaatcctcatgtactacaaaaagcggccgaagaagcagcacgtgtactggttgaccctgtgcccagttataaacaagtgaaacagttaaaatatgtcggaatggttctaaatgaggcgttacgtctatggccgacagcacccgcattctccctttatgctaaagaagacactgttttaggaggcgaataccccttggaaaagggcgacgaactaatggtgttgatcccacaattacacagagataaaacgatctggggggatgatgtagaagaatttaggcctgagaggttcgaaaacccatccgcgataccccagcatgctttcaaaccgttcgggaatggtcaaagggcatgcattggtcagcagtttgcactgcatgaggccaccctagtcctgagtatgatgctaaagcatttcgatttcgaagatcacaccaactatgagttagatatcaaggagaccctaacgcttaaaccggagggcttcgttgtaaaggcgaaatccaaaaagatcccattgggtggaattccctcaccctcaacagaacaatccgccaaaaaagttagaaagaaggcagaaaatgcccataataccccgttattagttctttacgggtctaacatgggaacggccgagggtacagccagggaccttgcagacattgctatgtcaaagggctttgctccgcaagtcgcaaccttagattcccacgcagggaacctgccgagagaaggagcggtgcttattgttaccgcttcatataatggtcatcctccggataacgccaaacagttcgttgactggctggatcaggccagcgcggacgaagttaagggggttcgttatagcgtgttcggttgcggagataaaaattgggccaccacgtatcagaaagtaccagcgttcatcgacgaaactttagccgccaagggtgcagagaacatagctgacagaggtgaagctgacgccagtgacgatttcgaagggacatatgaggaatggagagagcacatgtggagcgacgtcgccgcttattttaacctggatatagagaactctgaagacaataaatctactttgtctttgcagtttgtcgattctgccgcagacatgccgcttgctaaaatgcacggcgccttctccactaatgtcgtggcgagcaaggaattgcaacagccgggaagcgcgcgttccacccgtcacttggagattgagctgcccaaggaggcttcataccaggaaggcgaccacttaggagtaatacctaggaattatgaaggtatagttaacagagtcacggcgcgttttggcctggatgcgtcacagcagatccgtctagaggcagaggaggagaagttggctcatcttcccttggcgaagacggtcagtgtcgaggagctgcttcagtatgtcgagctgcaagatcccgtcaccaggacgcaacttcgtgcaatggccgcaaagaccgtgtgccctccacacaaagtggaactagaagctcttttagagaaacaagcgtacaaggagcaggtcctggccaagcgtttgacaatgcttgaactattggaaaagtacccagcgtgtgaaatgaaattctccgaatttatcgctctactgccgagcatcagaccccgttactactctatctcttccagtccgagggtagatgaaaagcaggcatctataacagtctccgtcgtctctggggaagcttggagtgggtacggggaatataaaggcatagcctccaattaccttgctgagctgcaggaaggtgacacaatcacatgtttcatttcaacccctcaaagtgaatttactctgccaaaggatccggagactccactaatcatggtcgggcctggaactggtgtagcgccttttagaggtttcgtccaagccagaaagcaactgaaagagcagggtcagtcattaggcgaagcccatttatactttggatgtagatctcctcatgaagattatctgtaccaggaagaactagaaaacgctcaaagcgaagggattattacactgcacacggccttcagccgtatgcctaatcagcccaaaacctatgtgcaacatgtaatggagcaagatggtaaaaagttaattgagttgttagatcagggcgcacatttttacatttgtggggatggctctcagatggcgcccgctgtggaagcaaccctgatgaagagttacgcagatgtacaccaggtcagtgaggcggatgcaaggctgtggttacagcaacttgaagaaaagggccgttacgctaaggacgtgtgggcgggatga

SEQ ID NO: 164 - protein, 491 aa, Rattus norvegicus
  REGION 1..491: note = 14HC_P450_31
MEPTILLLLALLVGFLLLLVRGHPKSRGNFPPGPRPLPLLGNLLQLDRGGLLNSFMQLREKYGDVFTVHLGPRPVVMLCGTDTIKEALVGQAEDFSGRGTIAVIEPIFKEYGVIFANGERWKALRRFSLATMRDFGMGKRSVEERIQEEAQCLVEELRKSQGAPLDPTFLFQCITANIICSIVFGERFDYTDRQFLRLLELFYRTFSLLSSFSSQVFEFFSGFLKYFPGAHRQISKNLQEILDYIGHIVEKHRATLDPSAPRDFIDTYLLRMEKEKSNHHTEFHHENLMISLLSLFFAGTETSSTTLRYGFLLMLKYPHVAEKVQKEIDQVIGSHRLPTLDDRSKMPYTDAVIHEIQRFSDLVPIGVPHRVTKDTMFRGYLLPKNTEVYPILSSALHDPQYFDHPDSFNPEHFLDANGALKKSEAFMPFSTGKRICLGEGIARNELFLFFTTILQNFSVSSHLAPKDIDLTPKESGIGKIPPTYQICFSAR

SEQ ID NO: 165 - DNA, 1476 bp, Rattus norvegicus
  source 1..1476: mol_type = genomic DNA
  misc_feature 1..1476: note = 14HC_P450_31
atggagcccaccatattgctactgttggcgttattagtcggatttttgctactgctagtgagaggacatccaaagtcccgtggcaacttcccacccggtccaaggccgttaccccttttgggtaatttattacaacttgataggggaggactattgaatagtttcatgcaactaagagagaaatacggtgatgtgttcacagttcacttggggccgagacctgtagtaatgctgtgtgggactgatactataaaagaggccttagtcgggcaagcggaagacttctcaggaagaggcaccatagcagtaattgaacctatcttcaaagaatacggagtcatattcgctaacggcgagcgttggaaggcgctgcgtagattctcacttgctactatgagggattttggcatggggaagcgtagcgtggaagagagaatccaagaggaggcacagtgcttagtagaggaactacgtaagtcccagggcgctccgttggacccaacgtttttgttccagtgcatcacggctaacatcatctgctccatagtctttggagaacgtttcgactacactgacaggcaatttttacgtttactggaactgttttaccgtacgttttcactactttcaagtttcagtagtcaggtctttgagtttttctctggattcttgaagtactttccaggggcccataggcagatcagtaaaaacctacaagaaattttagactacattggacatatcgtagagaagcatcgtgctacgcttgaccccagcgctccaagagactttatcgatacgtacctgcttaggatggaaaaagagaagtcaaatcatcacacagaattccatcacgaaaacctgatgatttccttgctatctttattcttcgcggggaccgagacaagtagcactacgctaaggtatgggtttttactaatgcttaagtacccccatgtagcggagaaagtgcaaaaagaaatagatcaggtcataggctcacatagacttcccactttggacgaccgtagcaagatgccctatacagacgctgttatacatgagatacaacgtttctccgatttggtacctataggggtaccacatagggtgacgaaggataccatgtttagagggtacctgttaccgaaaaacacagaggtctatcctattctttcttccgcccttcatgatccacaatactttgaccaccccgattcctttaacccggaacacttccttgacgctaatggtgcattaaagaaaagtgaggcatttatgccattctctaccggcaaacgtatttgcttaggggagggcatcgcccgtaacgagttattcttattctttacaactatcctgcaaaatttctctgtatcctcacatcttgcccctaaggacattgacctgacgcctaaagagtccggtatcggtaagattcctcctacttatcagatatgctttagtgcgagatga

SEQ ID NO: 166 - protein, 504 aa, Rattus norvegicus
  REGION 1..504: note = 14HC_P450_32
MDLLSALTLETWVLLAVILVLLYRLGTHRHGIFKKQGIPGPKPLPFLGTVLNYYKGLGRFDMECYKKYGKIWGLFDGQTPVFAIMDTEMIKNVLVKECFSVFTNRRDFGPVGIMGKAVSVAKDEEWKRYRALLSPTFTSGRLKEMFPIIEQYGDILVKYLKQEAETGKPVTMKKVFGAYSMDVITSTSFGVNVDSLNNPKDPFVEKTKKLLRFDFFDPLFLSVVLFPFLTPIYEMLNICMFPKDSIAFFQKFVHRIKETRLDSKHKHRVDFLQLMLNAHNNSKDEVSHKALSDVEIIAQSVIFIFAGYETTSSTLSFVLYFLATHPDIQKKLQEEIDGALPSKAPPTYDIVMEMEYLDMVLNETLRLYPIGNRLERVCKKDIELDGLFIPKGSVVTIPTYALHHDPQHWPKPEEFHPERFSKENKGSIDPYVYLPFGNGPRNCIGMRFALMNMKLALTKVLQNFSFQPCKETQIPLKLSRQAILEPEKPIVLKVLPRDAVINGA

SEQ ID NO: 167 - DNA, 1515 bp, Rattus norvegicus
  source 1..1515: mol_type = genomic DNA
  misc_feature 1..1515: note = 14HC_P450_32
atggatcttctgtccgcgctaactttagaaacgtgggtactactggcagtaatcctagtattattgtataggctagggactcacagacacggtattttcaagaagcaaggtatcccaggtccgaaaccactgccctttctgggtactgtccttaactattataaaggtctgggtaggttcgacatggaatgctataagaagtacggcaaaatttggggcttattcgacgggcaaacgcccgtgtttgccataatggataccgaaatgataaagaatgtcctagtcaaagaatgtttcagcgtttttacaaacaggcgtgattttgggcctgtcggaattatgggcaaggctgtaagcgtggctaaggatgaagagtggaagagatatagagccttactgagccctaccttcacttctggtagactaaaagaaatgtttccgataattgagcaatacggcgacatcctagttaaatacctaaaacaagaggccgagacgggcaaacccgtgactatgaaaaaggtattcggagcgtacagtatggatgttataacttctacctcttttggggtaaatgtcgacagtttaaacaaccccaaagaccccttcgttgaaaaaactaagaaattattaagattcgatttttttgaccctctattcttatccgttgtattattccccttcctgacaccgatatatgaaatgttaaatatctgtatgttcccgaaggacagtatagcgttttttcaaaaatttgtccatagaattaaggaaacgcgtctagatagcaagcataagcacagagttgactttttgcaactaatgctaaacgctcataacaactcaaaagacgaagtgtctcataaagcgcttagtgacgttgaaataatcgctcagagcgtcatatttatatttgctgggtacgaaaccacctcctcaactttgtcttttgtcctatatttccttgctacacatcctgatattcaaaaaaagttacaagaagagattgatggggcactgccatccaaggcaccaccaacctacgatattgtaatggagatggaatacctagacatggtgttgaacgagacattgagattgtatcccatcggtaatagacttgaaagagtgtgcaaaaaagacattgagcttgatggtctttttatcccgaagggttccgtagtgacaatccctacatacgcgctacatcatgatcctcaacattggccaaagccggaggaattccaccccgagcgtttttccaaagaaaacaaagggagtatagacccatatgtatatttaccattcgggaacggcccacgtaactgtatcggaatgaggtttgccctgatgaacatgaaattagcgttgaccaaagtccttcagaatttcagcttccagccttgtaaagaaacgcaaatccctctgaagctatcaagacaagcaatcctggaacccgaaaagccaattgtcttaaaggttctaccgagagacgcggtcataaatggtgcatga

SEQ ID NO: 168 - protein, 514 aa, Lichtheimia ramosa; identical to SEQ ID NO: 112
  REGION 1..514: note = 14HC_P450_5

SEQ ID NO: 169 - DNA, 1545 bp, Lichtheimia ramosa; identical to SEQ ID NO: 113
  source 1..1545: mol_type = genomic DNA
  misc_feature 1..1545: note = 14HC_P450_5

SEQ ID NO: 170 - protein, 514 aa, Lichtheimia ramosa
  REGION 1..514: note = 14HC_P450_33 (14HC_P450_5: A59D)
MTEIKEHIYRYRHYIGVAAAVALVCQQVYYRIFRIPKNLRHIPAIPYGQQLKALRSDEDLTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTLDALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 171 - DNA, 1545 bp, Lichtheimia ramosa
  source 1..1545: mol_type = genomic DNA
  misc_feature 1..1545: note = 14HC_P450_33 (14HC_P450_5: A59D)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgttagacgcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 172 - protein, 514 aa, Lichtheimia ramosa
  REGION 1..514: note = 14HC_P450_34 (14HC_P450_5: L188I)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTIDALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 173 - DNA, 1545 bp, Lichtheimia ramosa
  source 1..1545: mol_type = genomic DNA
  misc_feature 1..1545: note = 14HC_P450_34 (14HC_P450_5: L188I)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgatagacgcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 174 - protein, 514 aa, Lichtheimia ramosa
  REGION 1..514: note = 14HC_P450_35 (14HC_P450_5: L188I,D189E)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTIEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 175 - DNA, 1545 bp, Lichtheimia ramosa
  source 1..1545: mol_type = genomic DNA
  misc_feature 1..1545: note = 14HC_P450_35 (14HC_P450_5: L188I,D189E)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgatagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 176 - protein, 514 aa, Lichtheimia ramosa
  REGION 1..514: note = 14HC_P450_36 (14HC_P450_5: L188I,D189E,A59D)
MTEIKEHIYRYRHYIGVAAAVALVCQQVYYRIFRIPKNLRHIPAIPYGQQLKALRSDEDLTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTIEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 177 - DNA, 1545 bp, Lichtheimia ramosa
  source 1..1545: mol_type = genomic DNA
  misc_feature 1..1545: note = 14HC_P450_36 (14HC_P450_5: L188I,D189E,A59D)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgatagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 178 - protein, 514 aa, Lichtheimia ramosa
  REGION 1..514: note = 14HC_P450_37 (A59D,F102L,D181E,L188I,D189E,S208C,L325M )
MTEIKEHIYRYRHYIGVAAAVALVCQQVYYRIFRIPKNLRHIPAIPYGQQLKALRSDEDLTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHAMTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 179 - DNA, 1545 bp, Lichtheimia ramosa
  source 1..1545: mol_type = genomic DNA
  misc_feature 1..1545: note = 14HC_P450_37 (A59D,F102L,D181E,L188I,D189E,S208C,L325M )
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctatgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 180 - protein, 514 aa, Lichtheimia ramosa
  REGION 1..514: note = 14HC_P450_39 (E58K,A59D,F102L,D181E,L188I,D189E )
MTEIKEHIYRYRHYIGVAAAVALVCQQVYYRIFRIPKNLRHIPAIPYGQQLKALRSDKDLTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 181 - DNA, 1545 bp, Lichtheimia ramosa
  source 1..1545: mol_type = genomic DNA
  misc_feature 1..1545: note = 14HC_P450_39 (E58K,A59D,F102L,D181E,L188I,D189E )
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgataaggatcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 182 - protein, 514 aa, Lichtheimia ramosa
  REGION 1..514: note = 14HC_P450_41 (E58K,A59D,D181E,L188I,D189E,S208C)
MTEIKEHIYRYRHYIGVAAAVALVCQQVYYRIFRIPKNLRHIPAIPYGQQLKALRSDKDLTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 183 - DNA, 1545 bp, Lichtheimia ramosa
  source 1..1545: mol_type = genomic DNA
  misc_feature 1..1545: note = 14HC_P450_41 (E58K,A59D,D181E,L188I,D189E,S208C)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgataaggatcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 184 - protein, 514 aa, Lichtheimia ramosa
  REGION 1..514: note = 14HC_P450_43 (V17I)
MTEIKEHIYRYRHYIGIAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTLDALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 185 - DNA, 1545 bp, Lichtheimia ramosa
  source 1..1545: mol_type = genomic DNA
  misc_feature 1..1545: note = 14HC_P450_43 (V17I)
atgacagagatcaaggagcacatctacagataccgtcactatatcgggattgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgttagacgcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 186 - protein, 514 aa, Lichtheimia ramosa
  REGION 1..514: note = 14HC_P450_44 (F102L)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALDLMQKLTLDALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 187 - DNA, 1545 bp, Lichtheimia ramosa
  source 1..1545: mol_type = genomic DNA
  misc_feature 1..1545: note = 14HC_P450_44 (F102L)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgttagacgcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 188 - protein, 514 aa, Lichtheimia ramosa
  REGION 1..514: note = 14HC_P450_45 (D181E)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALELMQKLTLDALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 189 - DNA, 1545 bp, Lichtheimia ramosa
  source 1..1545: mol_type = genomic DNA
  misc_feature 1..1545: note = 14HC_P450_45 (D181E)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgttagacgcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 190 - protein, 514 aa, Lichtheimia ramosa
  REGION 1..514: note = 14HC_P450_46 (D189E)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTLEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 191 - DNA, 1544 bp, Lichtheimia ramosa
  source 1..1544: mol_type = genomic DNA
  misc_feature 1..1544: note = 14HC_P450_46 (D189E)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgttagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttacta

SEQ ID NO: 192 - protein, 514 aa, Lichtheimia ramosa
  REGION 1..514: note = 14HC_P450_47 (S280C)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTLDALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 193 - DNA, 1545 bp, Lichtheimia ramosa
  source 1..1545: mol_type = genomic DNA
  misc_feature 1..1545: note = 14HC_P450_47 (S280C)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgttagacgcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 194 - protein, 514 aa, Lichtheimia ramosa
  REGION 1..514: note = 14HC_P450_48 (L325M)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTLDALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHAMTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 195 - DNA, 1545 bp, Lichtheimia ramosa
  source 1..1545: mol_type = genomic DNA
  misc_feature 1..1545: note = 14HC_P450_48 (L325M)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgttagacgcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctatgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 196 - protein, 514 aa, Lichtheimia ramosa
  REGION 1..514: note = 14HC_P450_49 (L396I)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTLDALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDINVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 197 - DNA, 1545 bp, Lichtheimia ramosa
  source 1..1545: mol_type = genomic DNA
  misc_feature 1..1545: note = 14HC_P450_49 (L396I)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgttagacgcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatataaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 198 - protein, 514 aa, Lichtheimia ramosa
  REGION 1..514: note = 14HC_P450_50 (L188I,D189E,S208C)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 199 - DNA, 1545 bp, Lichtheimia ramosa
  source 1..1545: mol_type = genomic DNA
  misc_feature 1..1545: note = 14HC_P450_50 (L188I,D189E,S208C)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 200 - protein, 514 aa, Lichtheimia ramosa
  REGION 1..514: note = 14HC_P450_51 ( F102L, L188I, D189E)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALDLMQKLTIEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 201 - DNA, 1545 bp, Lichtheimia ramosa
  source 1..1545: mol_type = genomic DNA
  misc_feature 1..1545: note = 14HC_P450_51 ( F102L, L188I, D189E)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgatagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 202 - protein, 514 aa, Lichtheimia ramosa
  REGION 1..514: note = 14HC_P450_52 ( V17I, L1881, D189E)
MTEIKEHIYRYRHYIGIAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTIEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 203 - DNA, 1545 bp, Lichtheimia ramosa
  source 1..1545: mol_type = genomic DNA
  misc_feature 1..1545: note = 14HC_P450_52 ( V17I, L1881, D189E)
atgacagagatcaaggagcacatctacagataccgtcactatatcgggattgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgatagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 204 - protein, 514 aa, Lichtheimia ramosa
  REGION 1..514: note = 14HC_P450_53 ( L188I, D189E,L325M)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTIEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHAMTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 205 - DNA, 1545 bp, Lichtheimia ramosa
  source 1..1545: mol_type = genomic DNA
  misc_feature 1..1545: note = 14HC_P450_53 ( L188I, D189E,L325M)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgatagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctatgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 206 - protein, 514 aa, Lichtheimia ramosa
  REGION 1..514: note = 14HC_P450_54 ( D181E, L188I, D189E)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 207 - DNA, 1545 bp, Lichtheimia ramosa
  source 1..1545: mol_type = genomic DNA
  misc_feature 1..1545: note = 14HC_P450_54 ( D181E, L188I, D189E)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 208 - protein, 514 aa, Lichtheimia ramosa
  REGION 1..514: note = 14HC_P450_55 ( D181E, L188I, D189E, S208C)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 209 - DNA, 1545 bp, Lichtheimia ramosa
  source 1..1545: mol_type = genomic DNA
  misc_feature 1..1545: note = 14HC_P450_55 ( D181E, L188I, D189E, S208C)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 210 - protein, 514 aa, Lichtheimia ramosa
  REGION 1..514: note = 14HC_P450_56 ( D181E, L188I, D189E, S208C, L325M)
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHAMTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 211 - DNA, 1545 bp, Lichtheimia ramosa
  source 1..1545: mol_type = genomic DNA
  misc_feature 1..1545: note = 14HC_P450_56 ( D181E, L188I, D189E, S208C, L325M)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctatgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 212 - protein, 514 aa, Lichtheimia ramosa
  REGION 1..514: note = 14HC_P450_57 ( V17I, D181E, L188I, D189E, S208C)
MTEIKEHIYRYRHYIGIAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 213 - DNA, 1545 bp, Lichtheimia ramosa
  source 1..1545: mol_type = genomic DNA
  misc_feature 1..1545: note = 14HC_P450_57 ( V17I, D181E, L188I, D189E, S208C)
atgacagagatcaaggagcacatctacagataccgtcactatatcgggattgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 214 - protein, 514 aa, Lichtheimia ramosa
  REGION 1..514: note = 14HC_P450_58 ( A59D, D181E, L188I, D189E, S208C)
MTEIKEHIYRYRHYIGVAAAVALVCQQVYYRIFRIPKNLRHIPAIPYGQQLKALRSDEDLTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 215 - DNA, 1545 bp, Lichtheimia ramosa
  source 1..1545: mol_type = genomic DNA
  misc_feature 1..1545: note = 14HC_P450_58 ( A59D, D181E, L188I, D189E, S208C)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 216 - protein, 514 aa, Lichtheimia ramosa
  REGION 1..514: note = 14HC_P450_59 ( A59D, D181E, L188I, D189E, S208C, L325M)
MTEIKEHIYRYRHYIGVAAAVALVCQQVYYRIFRIPKNLRHIPAIPYGQQLKALRSDEDLTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHAMTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 217 - DNA, 1545 bp, Lichtheimia ramosa
  source 1..1545: mol_type = genomic DNA
  misc_feature 1..1545: note = 14HC_P450_59 ( A59D, D181E, L188I, D189E, S208C, L325M)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctatgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 218 - protein, 514 aa, Lichtheimia ramosa
  REGION 1..514: note = 14HC_P450_60 ( A59D, F102L, D181E, L188I, D189E, S208C)
MTEIKEHIYRYRHYIGVAAAVALVCQQVYYRIFRIPKNLRHIPAIPYGQQLKALRSDEDLTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 219 - DNA, 1545 bp, Lichtheimia ramosa
  source 1..1545: mol_type = genomic DNA
  misc_feature 1..1545: note = 14HC_P450_60 ( A59D, F102L, D181E, L188I, D189E, S208C)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 220 - protein, 514 aa, Lichtheimia ramosa
  REGION 1..514: note = 14HC_P450_61 ( V17I, A59D, L188I, D189E, S208C, D181E, L325M)
MTEIKEHIYRYRHYIGIAAAVALVCQQVYYRIFRIPKNLRHIPAIPYGQQLKALRSDEDLTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHAMTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 221 - DNA, 1545 bp, Lichtheimia ramosa
  source 1..1545: mol_type = genomic DNA
  misc_feature 1..1545: note = 14HC_P450_61 ( V17I, A59D, L188I, D189E, S208C, D181E, L325M)
atgacagagatcaaggagcacatctacagataccgtcactatatcgggattgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctatgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 222 - protein, 514 aa, Lichtheimia ramosa
  REGION 1..514: note = 14HC_P450_62 ( V17I, A59D, F102L, D181E, L188I, D189E, S208C)
MTEIKEHIYRYRHYIGIAAAVALVCQQVYYRIFRIPKNLRHIPAIPYGQQLKALRSDEDLTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 223 - DNA, 1545 bp, Lichtheimia ramosa
  source 1..1545: mol_type = genomic DNA
  misc_feature 1..1545: note = 14HC_P450_62 ( V17I, A59D, F102L, D181E, L188I, D189E, S208C)
atgacagagatcaaggagcacatctacagataccgtcactatatcgggattgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 224 - protein, 514 aa, Lichtheimia ramosa
  REGION 1..514: note = 14HC_P450_63 (A59D,F102L,D181E,L188I,D189E)
MTEIKEHIYRYRHYIGVAAAVALVCQQVYYRIFRIPKNLRHIPAIPYGQQLKALRSDEDLTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 225 - DNA, 1545 bp, Lichtheimia ramosa
  source 1..1545: mol_type = genomic DNA
  misc_feature 1..1545: note = 14HC_P450_63 (A59D,F102L,D181E,L188I,D189E)
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 226 - protein, 514 aa, Lichtheimia ramosa
  REGION 1..514: note = 14HC_P450_64 (V17I,A59D,F102L,D181E,L188I,D189E)
MTEIKEHIYRYRHYIGIAAAVALVCQQVYYRIFRIPKNLRHIPAIPYGQQLKALRSDEDLTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 227 - DNA, 1545 bp, Lichtheimia ramosa
  source 1..1545: mol_type = genomic DNA
  misc_feature 1..1545: note = 14HC_P450_64 (V17I,A59D,F102L,D181E,L188I,D189E)
atgacagagatcaaggagcacatctacagataccgtcactatatcgggattgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 228 - protein, 514 aa, Lichtheimia ramosa
  REGION 1..514: note = 14HC_P450_65 (V17I,A59D,F102L,D181E,L188I,D189E,S208C,L325M )
MTEIKEHIYRYRHYIGIAAAVALVCQQVYYRIFRIPKNLRHIPAIPYGQQLKALRSDEDLTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHAMTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 229 - DNA, 1545 bp, Lichtheimia ramosa
  source 1..1545: mol_type = genomic DNA
  misc_feature 1..1545: note = 14HC_P450_65 (V17I,A59D,F102L,D181E,L188I,D189E,S208C,L325M )
atgacagagatcaaggagcacatctacagataccgtcactatatcgggattgcggctgctgtcgctttggtgtgccaacaagtgtattacaggatctttcgtatccctaagaacctgaggcacatacctgccattccgtacggtcaacaactaaaggcattgaggtccgatgaggatcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctatgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 230 - protein, 514 aa, Lichtheimia ramosa
  REGION 1..514: note = 14HC_P450_66 (E58K,A59D,F102L,D181E,L188I,D189E,S208C,L325M )
MTEIKEHIYRYRHYIGVAAAVALVCQQVYYRIFRIPKNLRHIPAIPYGQQLKALRSDKDLTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHAMTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 231 - DNA, 1545 bp, Lichtheimia ramosa
  source 1..1545: mol_type = genomic DNA
  misc_feature 1..1545: note = 14HC_P450_66 (E58K,A59D,F102L,D181E,L188I,D189E,S208C,L325M )
atgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgataaggatcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctatgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 232 - protein, 514 aa, Lichtheimia ramosa
  REGION 1..514: note = 14HC_P450_67 ( V17I, E58K, A59D,F102L,D181E,L188I,D189E,S208C)
MTEIKEHIYRYRHYIGIAAAVALVCQQVYYRIFRIPKNLRHIPAIPYGQQLKALRSDKDLTSRTKRLVFPLLSKCNGVYLNRMPFKWTIYVADPEIARAILLKPEFGHKTRSVTDSLDKNSSLFEFVGDDNIAIVNGHEWKEQRKIMNPAFHRATPVGMFGSLMPKVFRLVEEQPTLPALELMQKLTIEALGKSVFGFEFGALDDPDCVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 233 - DNA, 1545 bp, Lichtheimia ramosa
  source 1..1545: mol_type = genomic DNA
  misc_feature 1..1545: note = 14HC_P450_67 ( V17I, E58K, A59D,F102L,D181E,L188I,D189E,S208C)
atgacagagatcaaggagcacatctacagataccgtcactatatcgggattgcggctgctgtcgctttggtgtgccaacaagtgtattacaggatctttcgtatccctaagaacctgaggcacatacctgccattccgtacggtcaacaactaaaggcattgaggtccgataaggatcttaccagcaggacgaagcgtttagtattccctttgctatctaagtgtaatggggtataccttaatagaatgccatttaagtggacaatttatgtcgcggatccagaaatcgcgagggcgattcttttaaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagaattaatgcaaaagttaacgatagaggcacttgggaagtcagtattcggctttgaattcggcgcactagatgaccccgactgtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactag

SEQ ID NO: 234 - protein, 706 aa, Lichtheimia ramosa
  REGION 1..706: note = 14HC_CPR_1
MAQSPPALDTLDIVFLGTIGLGTIAWFARRQIAERLFGSTSSSDAKSNGHATPAPPKRERNFVKIMQEQGRKVIFFYGSQTGTAEDYASRLAKECSQKYGVNCMTADLELYDLSYLDTVPEDCLVFFVMATYGEGEPTDNAVDFWDVLSEEEPQFSEAEGDKPLQNLRYLVFGLGNKTYEHYNAVARNVDKRLEVLGAHRIHERGEGDDDGSLEEDFLAWQENMWPAFCEALGVDESNAHSGPRQATYSVEELVDVNMDDVYLGELAEKPKEGARVIYDAKRPFNAPIAISQDLFTNTDRHCLHMEIDISDSNLSYQTGDHIAIWPTNSENEVARLASILGLADKLDTAINVKAIDPAASKKYPFPCPATYRAIFRHYLDICAAVSRQSLMAFVEYAPTEESKERLRQLAKDKDEYRLTVGEAVRNLGEVLEIVAGNDVKPGFFSSVPFDLVVESISRLQPRYYSISSSAKESPKKIAVTAVTLSYQPDPTPQRTVYGVNTNYLWRIHTASKQQSTEADLPTYDLSGPRNALHGTKLPVHVRRSQFKLPRNPTVPVIMVGPGTGVAPFRGFVRERALQKSEGKPVGPTLLFFGCRNSEQDFLYKDEWPALFDTLGESSRIITAFSRETAQKVYVQHRLQENGQEVWGLLQRGAYIYVCGDAKNMARDVQQTFVNFGIEFGGLSDDKAHDFVKNLRNTGRYQEDVWS

SEQ ID NO: 235 - DNA, 2121 bp, Lichtheimia ramosa
  source 1..2121: mol_type = genomic DNA
  misc_feature 1..2121: note = 14HC_CPR_1
atggctcaatctccaccagcattggatactttggatatcgttttcttgggtactatcggtttgggtacaattgcttggtttgctagaaggcaaattgctgaaagattattcggttccacctcttcctctgatgctaaatctaatggtcatgctactccagctccaccaaaaagagaaagaaacttcgttaagatcatgcaagagcaaggtagaaaggtcattttcttctacggttctcaaactggtactgctgaagattacgcttctagattggctaaagaatgctctcaaaagtacggtgttaactgtatgactgctgatttggagttgtacgacttgtcttacttggatactgttccagaagattgcctggtttttttcgttatggctacttatggtgaaggtgaacctactgataatgctgttgatttctgggatgtcttgtctgaagaagaaccacaattttctgaagccgaaggtgataagccattgcaaaatttgagatacttggttttcggcttgggtaacaagacttacgaacattataacgctgttgccagaaacgttgacaagagattggaagttttgggtgctcatagaattcatgaacgtggtgagggtgatgatgatggttctttggaagaagattttttggcctggcaagaaaacatgtggccagctttttgtgaagctttgggtgttgatgaatctaacgctcattctggtccaagacaagctacttattctgttgaagaattggtcgatgtcaacatggatgatgtttacttgggtgaattggccgaaaaacctaaagaaggtgctagagttatctacgatgctaagaggccttttaatgctccaattgccatttctcaagacttgttcactaacaccgatagacattgcttgcacatggaaatcgatatctccgattccaacttgtcataccaaaccggtgatcatattgctatttggccaactaactccgaaaacgaagttgctagactggcttctattttgggtttagctgataagttggataccgccatcaatgttaaggctattgatccagctgcttccaaaaagtacccatttccttgtccagctacttacagagctattttcagacattacttggacatttgcgctgccgtttctagacaatctttgatggcttttgttgaatacgctccaaccgaagaatccaaagaaagattgagacaattggccaaggataaggatgagtacagattgactgttggtgaagctgttagaaatttgggtgaagtattggaaatcgttgctggtaatgatgttaagccaggtttcttttcttccgttccattcgatttggttgtcgaatctatttccagattgcaacctaggtactactccatttcttcatctgcaaaagagtccccaaaaaagatcgctgttactgctgttacattgtcctatcaaccagatccaactccacaaagaactgtttatggtgtcaacactaattacttgtggcgtattcataccgcctctaagcaacaatctactgaagctgatttgccaacctatgatttgtcaggtccaagaaatgctttacatggtactaagttgccagttcacgttagaagatctcaattcaagttgccaagaaacccaaccgttccagttattatggttggtccaggtactggtgttgctccttttagaggttttgttagagaaagagccttgcagaaatctgaaggtaaaccagttggtccaactttgttgtttttcggttgtagaaactccgaacaggacttcttgtacaaagatgaatggcctgctttgtttgacaccttaggtgaatcctctagaattattaccgccttctcaagagaaaccgctcaaaaagtttacgtccagcatagattgcaagagaacggtcaagaagtttggggtttgttgcaaaggggtgcttatatctatgtttgtggtgatgcaaagaacatggccagagatgttcaacaaactttcgttaacttcggtatcgaattcggtggtttgtctgatgataaggctcatgattttgtcaagaacttgagaaacaccggtagataccaagaagatgtttggtcttga

SEQ ID NO: 236 - protein, 707 aa, Thamnostylum piriforme
  REGION 1..707: note = 14HC_CPR_2
MSKKPTTFAISPLDLLLLGTLGIGALLYITRKLRAAPPPAAGDAARPSDKAPSNKPERNFVKLMEQQKRRVIFFYGSQTGTAEDYASRLAKESSQKYGVSSMAADIELYDLSYLDTVPEDKLVVFVMATYGEGEPTDNAVDFWETLTDEAPMFSLGEELAPLRNLNYIVFGLGNKTYEHYNEVARVLDKRLAALGATRHGVRGEGDDDASLEEDFLAWQEDMWPAFCAALNVDESNAQAGPRQAMYGVKELEGHDENGLFLGELGEWLQPEQLQKQTAVHDAKHPYLAPIAASRDLFSHAADRHCLHLEVDIASSNLTYQTGDHIAIWPTNNDVEVLRLATVLGLADKLDTAIMVKALDSAASKKYPFPVPTTYRAAFKHYLDICSPASRQTLISLVDYAPTASSKDALHKLATDKDAYKTQVSEAARNLAEVLELCQDDEGTTAAPGFFSTVPFDLIVESVSRLQPRYYSISSSSLTSPKSVAVTAVTLSYQPSTTKSRTVYGVNTNYLWRIHAPHDASVPAYDLDGPRNTLQGKKVPVHIRRSTFKLPRNTKLPVIMVGPGTGVAPFRGFIHDRVHQKQSGKEVGPTVLFYGCRHSKQDFLYAEEWPALFEALGEGSQLITAFSRESAQKVYVQHRLKEHGEQMWKYIEQGAYIYVCGDAKNMARDVQQTFIEFAQALGNKTESQAQDYVKNLRNTGRYQEDVWS

SEQ ID NO: 237 - DNA, 2124 bp, Thamnostylum piriforme
  source 1..2124: mol_type = genomic DNA
  misc_feature 1..2124: note = 14HC_CPR_2
atgtctaaaaaaccaactacttttgctatttctccattggatttgttgttgttgggtactttgggtattggtgctttgttgtatattactagaaaattgagagctgctccaccaccagctgctggtgatgctgctagaccatctgataaagctccatctaataaaccagaaagaaattttgttaaattgatggaacaacaaaaaagaagagttatttttttttatggttctcaaactggtactgctgaagattatgcttctagattggctaaagaatcttctcaaaaatatggtgtttcttctatggctgctgatattgaattgtatgatttgtcttatttggatactgttccagaagataaattggttgtttttgttatggctacttatggtgaaggtgaaccaactgataatgctgttgatttttgggaaactttgactgatgaagctccaatgttttctttgggtgaagaattggctccattgagaaatttgaattatattgtttttggtttgggtaataaaacttatgaacattataatgaagttgctagagttttggataaaagattggctgctttgggtgctactagacatggtgttagaggtgaaggtgatgatgatgcttctttggaagaagattttttggcttggcaagaagatatgtggccagctttttgtgctgctttgaatgttgatgaatctaatgctcaagctggtccaagacaagctatgtatggtgttaaagaattggaaggtcatgatgaaaatggtttgtttttgggtgaattgggtgaatggttgcaaccagaacaattgcaaaaacaaactgctgttcatgatgctaaacatccatatttggctccaattgctgcttctagagatttgttttctcatgctgctgatagacattgtttgcatttggaagttgatattgcttcttctaatttgacttatcaaactggtgatcatattgctatttggccaactaataatgatgttgaagttttgagattggctactgttttgggtttggctgataaattggatactgctattatggttaaagcattggattctgctgcttctaaaaaatatccatttccagttccaactacttatagagctgcttttaaacattatttggatatttgttctccagcttctagacaaactttgatttctttggttgattatgctccaactgcttcttctaaagatgctttgcataaattggctactgataaagatgcttataaaactcaagtttctgaagctgctagaaatttggctgaagttttggaattgtgtcaagatgatgaaggtactactgctgctccaggttttttttctactgttccatttgatttgattgttgaatctgtttctagattgcaaccaagatattattctatttcttcttcttctttgacttctccaaaatctgttgctgttactgctgttactttgtcttatcaaccatctactactaaatctagaactgtttatggtgttaatactaattatttgtggagaatccatgctccacatgatgcttctgttccagcttatgatttggatggtccaagaaatactttgcaaggtaaaaaagttccagttcatattagaagatctacttttaaattgccaagaaatactaaattgccagttattatggttggtccaggtactggtgttgctccatttagaggttttattcatgatagagttcatcaaaaacaatctggtaaagaagttggtccaactgttttgttttatggttgtagacattctaaacaagattttttgtatgctgaagaatggccagctttgtttgaagcattgggtgaaggttctcaattgattactgctttttctagagaatctgctcaaaaagtttatgttcaacatagattgaaagaacatggtgaacaaatgtggaaatatattgaacaaggtgcttatatttatgtttgtggtgatgctaaaaatatggctagagatgttcaacaaacttttattgaatttgctcaagcattgggtaataaaactgaatctcaagctcaagattatgttaaaaatttgagaaatactggtagataccaagaagatgtttggtcttag

SEQ ID NO: 238 - protein, 787 aa, Homo sapiens
  REGION 1..787: note = 14HC_CPR_3
MINMGDSHVDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWSRRAMAEQSDEAVKYYTLEEIQKHNHSKSTWLILHHKVYDLTKFLEEHPGGEEVLREQAGGDATENFEDVGHSTDAREMSKTFIIGELHPDDRPKLNKPPETLITTID

SEQ ID NO: 239 - DNA, 2364 bp, Homo sapiens
  source 1..2364: mol_type = genomic DNA
  misc_feature 1..2364: note = 14HC_CPR_3
atgatcaacatgggtgattctcacgttgatacctcttctactgtttctgaagctgttgctgaagaagtctctttgttctctatgaccgatatgatcctgttctctttgatcgttggtttgttgacttactggttcctgttcagaaagaagaaagaggaagttccagagttcaccaagattcaaactttgacctcttccgtcagagaatcttcattcgttgagaagatgaagaaaaccggtagaaacatcatcgtgttctacggttctcaaactggtacagctgaagaatttgctaacagattgtctaaggatgcccatagatatggtatgagaggtatgtctgctgatccagaagaatacgatttggctgatttgtcatccttgccagaaattgataatgccttggttgttttctgtatggctacttatggtgaaggtgatccaactgataatgctcaagatttctacgactggttgcaagaaactgatgttgatttgtctggtgttaagttcgctgttttcggtttgggtaacaagacttacgaacattttaacgctatgggcaagtacgttgacaagagattggaacaattgggtgcccaaagaatcttcgaattaggtttgggtgatgatgacggtaatttggaagaagatttcatcacttggagagaacaattttggccagctgtttgtgaacattttggtgttgaagctactggtgaggaatcctctattagacaatacgaattggttgttcacaccgatattgatgctgctaaagtctatatgggtgagatgggtagattgaagtcttacgaaaatcaaaagccaccattcgatgctaagaatccattcttggctgctgttactaccaacagaaaattgaatcagggtactgaaagacacttgatgcacttggaattggatatctccgattctaagatcaggtacgaatctggtgatcatgttgctgtttatccagctaatgattccgccttggttaatcaattgggtaagattttgggtgccgatttggatgttgttatgtccttgaacaacttggacgaagagtccaacaagaaacatccatttccatgtccaacctcttacagaactgctttgacttattacctggacattaccaatccaccaagaactaatgtcttgtacgaattagctcaatacgcctctgaaccatccgaacaagaattattgagaaagatggcttcctcatccggtgaaggtaaagaattatacttgtcctgggttgttgaagccagaaggcatattttggctatattgcaagattgcccatccttaagaccaccaattgatcatttgtgcgaactgttgccaagattgcaagctagatattactccattgcctcctcatctaaggttcatccaaactctgttcatatttgcgccgttgttgttgaatacgaaacaaaagctggtaggattaacaagggtgttgctactaattggttgagagctaaagaacctgctggtgaaaatggtggtagagctttggttccaatgttcgttagaaagtcccaattcagattgccattcaaagctactaccccagttattatggttggtccaggtactggtgttgctccttttattggtttcattcaagaaagagcctggttgagacaacagggtaaagaagttggagaaaccttgttgtattacggttgtagaagatccgacgaggattacttgtatagagaagaattggctcagttccatagagatggtgctttgacacaattgaacgttgccttttctagagaacagtcccataaggtttacgtccaacacttgttgaaacaagacagagaacatctgtggaagttgattgaaggtggtgctcatatctacgtttgtggtgatgctagaaatatggctagagatgttcaaaacaccttctacgatattgtcgctgaattaggtgctatggaacatgctcaagctgttgactacatcaagaaactaatgaccaagggcagatactcattggatgtttggtctagaagagctatggccgaacaatctgatgaagctgttaagtactacaccttggaagaaatccaaaagcacaaccattctaagtctacctggttgatcttgcatcacaaggtttacgatttgaccaagttcttagaagaacatccaggtggtgaagaagtcttgagagaacaagctggtggtgatgctactgaaaattttgaagatgttggtcattccaccgatgccagagaaatgtctaagactttcattatcggtgaattgcacccagatgatagaccaaaattgaacaaaccaccagaaaccttgattaccaccatcgattaa

SEQ ID NO: 240 - protein, 692 aa, Arabidopsis thaliana [sequence omitted]

SEQ ID NO: 241 - DNA, 2079 bp, Arabidopsis thaliana
  source 1..2079: mol_type = genomic DNA
  misc_feature 1..2079: note = 14HC_CPR_4
atgacttctgctttgtatgcttccgatttgtttaagcagctcaagtcaattatggggacagattcgttatccgacgatgttgtacttgtgattgcaacgacgtctttggcactagtagctggatttgtggtgttgttatggaagaaaacgacggcggatcggagcggggagctgaagcctttgatgatccctaagtctcttatggctaaggacgaggatgatgatttggatttgggatccgggaagactagagtctctatcttcttcggtacgcagactggaacagctgagggatttgctaaggcattatccgaagaaatcaaagcgagatatgaaaaagcagcagtcaaagtcattgacttggatgactatgctgccgatgatgaccagtatgaagagaaattgaagaaggaaactttggcatttttctgtgttgctacttatggagatggagagcctactgacaatgctgccagattttacaaatggtttacggaggaaaatgaacgggatataaagcttcaacaactagcatatggtgtgtttgctcttggtaatcgccaatatgaacattttaataagatcgggatagttcttgatgaagagttatgtaagaaaggtgcaaagcgtcttattgaagtcggtctaggagatgatgatcagagcattgaggatgattttaatgcctggaaagaatcactatggtctgagctagacaagctcctcaaagacgaggatgataaaagtgtggcaactccttatacagctgttattcctgaataccgggtggtgactcatgatcctcggtttacaactcaaaaatcaatggaatcaaatgtggccaatggaaatactactattgacattcatcatccctgcagagttgatgttgctgtgcagaaggagcttcacacacatgaatctgatcggtcttgcattcatctcgagttcgacatatccaggacgggtattacatatgaaacaggtgaccatgtaggtgtatatgctgaaaatcatgttgaaatagttgaagaagctggaaaattgcttggccactctttagatttagtattttccatacatgctgacaaggaagatggctccccattggaaagcgcagtgccgcctcctttccctggtccatgcacacttgggactggtttggcaagatacgcagaccttttgaaccctcctcgaaagtctgcgttagttgccttggcggcctatgccactgaaccaagtgaagccgagaaacttaagcacctgacatcacctgatggaaaggatgagtactcacaatggattgttgcaagtcagagaagtcttttagaggtgatggctgcttttccatctgcaaaacccccactaggtgtattttttgctgcaatagctcctcgtctacaacctcgttactactccatctcatcctcgccaagattggcgccaagtagagttcatgttacatccgcactagtatatggtccaactcctactggtagaatccacaagggtgtgtgttctacgtggatgaagaatgcagttcctgcggagaaaagtcatgaatgtagtggagccccaatctttattcgagcatctaatttcaagttaccatccaacccttcaactccaatcgttatggtgggacctgggactgggctggcaccttttagaggttttctgcaggaaaggatggcactaaaagaagatggagaagaactaggttcatctttgctcttctttgggtgtagaaatcgacagatggactttatatacgaggatgagctcaataattttgttgatcaaggcgtaatatctgagctcatcatggcattctcccgtgaaggagctcagaaggagtatgttcaacataagatgatggagaaggcagcacaagtttgggatctaataaaggaagaaggatatctctatgtatgcggtgatgctaagggcatggcgagggacgtccaccgaactctacacaccattgttcaggagcaggaaggtgtgagttcgtcagaggcagaggctatagttaagaaacttcaaaccgaaggaagatacctcagagatgtctggtga

SEQ ID NO: 242 - protein, 707 aa, Thamnostylum piriforme
  REGION 1..707: note = 14HC_CPR_2
MSKKPTTFAISPLDLLLLGTLGIGALLYITRKLRAAPPPAAGDAARPSDKAPSNKPERNFVKLMEQQKRRVIFFYGSQTGTAEDYASRLAKESSQKYGVSSMAADIELYDLSYLDTVPEDKLVVFVMATYGEGEPTDNAVDFWETLTDEAPMFSLGEELAPLRNLNYIVFGLGNKTYEHYNEVARVLDKRLAALGATRHGVRGEGDDDASLEEDFLAWQEDMWPAFCAALNVDESNAQAGPRQAMYGVKELEGYDENGLFLGELGEWLQPEQLQKQTAVHDAKHPYLAPIAASRDLFSHAADRHCLHLEVDIASSNLTYQTGDHIAIWPTNNDVEVLRLATVLGLADKLDTAIMVKALDSAASKKYPFPVPTTYRAAFKHYLDICSPASRQTLISLVDYAPTASSKDALHKLATDKDAYKTQVSEAARNLAEVLELCQDDEGTTAAPGFFSTVPFDLIVESVSRLQPRYYSISSSSLTSPKSVAVTAVTLSYQPSTTKSRTVYGVNTNYLWRIHAPHDASVPAYDLDGPRNTLQGKKVPVHIRRSTFKLPRNTKLPVIMVGPGTGVAPFRGFIHDRVHQKQSGKEVGPTVLFYGCRHSKQDFLYAEEWPALFEALGEGSQLITAFSRESAQKVYVQHRLKEHGEQMWKYIEQGAYIYVCGDAKNMARDVQQTFIEFAQALGNKTESQAQDYVKNLRNTGRYQEDVWS

SEQ ID NO: 243 - DNA, 2124 bp, Thamnostylum piriforme
  source 1..2124: mol_type = genomic DNA
  misc_feature 1..2124: note = 14HC_CPR_2
atgtctaaaaaaccaactacttttgctatttctccattggatttgttgttgttgggtactttgggtattggtgctttgttgtatattactagaaaattgagagctgctccaccaccagctgctggtgatgctgctagaccatctgataaagctccatctaataaaccagaaagaaattttgttaaattgatggaacaacaaaaaagaagagttatttttttttatggttctcaaactggtactgctgaagattatgcttctagattggctaaagaatcttctcaaaaatatggtgtttcttctatggctgctgatattgaattgtatgatttgtcttatttggatactgttccagaagataaattggttgtttttgttatggctacttatggtgaaggtgaaccaactgataatgctgttgatttttgggaaactttgactgatgaagctccaatgttttctttgggtgaagaattggctccattgagaaatttgaattatattgtttttggtttgggtaataaaacttatgaacattataatgaagttgctagagttttggataaaagattggctgctttgggtgctactagacatggtgttagaggtgaaggtgatgatgatgcttctttggaagaagattttttggcttggcaagaagatatgtggccagctttttgtgctgctttgaatgttgatgaatctaatgctcaagctggtccaagacaagctatgtatggtgttaaagaattggaaggttatgatgaaaatggtttgtttttgggtgaattgggtgaatggttgcaaccagaacaattgcaaaaacaaactgctgttcatgatgctaaacatccatatttggctccaattgctgcttctagagatttgttttctcatgctgctgatagacattgtttgcatttggaagttgatattgcttcttctaatttgacttatcaaactggtgatcatattgctatttggccaactaataatgatgttgaagttttgagattggctactgttttgggtttggctgataaattggatactgctattatggttaaagcattggattctgctgcttctaaaaaatatccatttccagttccaactacttatagagctgcttttaaacattatttggatatttgttctccagcttctagacaaactttgatttctttggttgattatgctccaactgcttcttctaaagatgctttgcataaattggctactgataaagatgcttataaaactcaagtttctgaagctgctagaaatttggctgaagttttggaattgtgtcaagatgatgaaggtactactgctgctccaggttttttttctactgttccatttgatttgattgttgaatctgtttctagattgcaaccaagatattattctatttcttcttcttctttgacttctccaaaatctgttgctgttactgctgttactttgtcttatcaaccatctactactaaatctagaactgtttatggtgttaatactaattatttgtggagaatccatgctccacatgatgcttctgttccagcttatgatttggatggtccaagaaatactttgcaaggtaaaaaagttccagttcatattagaagatctacttttaaattgccaagaaatactaaattgccagttattatggttggtccaggtactggtgttgctccatttagaggttttattcatgatagagttcatcaaaaacaatctggtaaagaagttggtccaactgttttgttttatggttgtagacattctaaacaagattttttgtatgctgaagaatggccagctttgtttgaagcattgggtgaaggttctcaattgattactgctttttctagagaatctgctcaaaaagtttatgttcaacatagattgaaagaacatggtgaacaaatgtggaaatatattgaacaaggtgcttatatttatgtttgtggtgatgctaaaaatatggctagagatgttcaacaaacttttattgaatttgctcaagcattgggtaataaaactgaatctcaagctcaagattatgttaaaaatttgagaaatactggtagataccaagaagatgtttggtcttag

SEQ ID NO: 244 - protein, 706 aa, Lichtheimia ramosa; identical to SEQ ID NO: 234
  REGION 1..706: note = 14HC_CPR_1

SEQ ID NO: 245 - DNA, 2121 bp, Lichtheimia ramosa; identical to SEQ ID NO: 235
  source 1..2121: mol_type = genomic DNA
  misc_feature 1..2121: note = 14HC_CPR_1

SEQ ID NO: 246 - protein, 701 aa, Thamnostylum piriforme
  REGION 1..701: note = 14HC_CPR_5
MAQSSPALLDSLDIVFLGTIGLGTIAWFARRQIAERIFGSKDDANKNVGNGNAPTAPKRERNFVKVMQEQGRKVIFFYGSQTGTAEDYASRLAKECSQKYGVSCMTADIELYDLTYLDTVPEDFLVFFIMATYGEGEPTDNAVDFWEQLTEEEPQFSEGDTLGNLRYVVFGLGNKTYEHYNEVARRMDKLLTKLGAKRIGERGEGDDDASLEEDFLAWQDSMWPAFCDALGVDESNGSSGPRQAMYAVEELEGQEAVYLGELGEKPKEGVKVVYDAKRPYNAPLVSQDLFKNTDRHCLHIDIDVSDSNLSYQTGDHIAIWPTNSDDEVARLASLLGLTDKLDTSVMVKAIDSTASKQYPFPVPATYRSIFRHYLDICAPASRQTLMSLVEYAPTEASKEALRLLSKDKDEYRLKVGEAVRNLGEVLELAAGADARPGLFSTVPFDLIVESVSRLQPRYYSISSSAKESPKVIAVTAVTLTYNPDPTPERTVYGVNTNYLWQIHAAKHNVNDGQRYPTYDLAGPRNALQGAKVPVHIRRSQFKLPRNPTVPVIMVGPGTGVAPFRGFVRERAAQKTDGKPVGPTLLFFGCRNSQQDFLYKDEWPELFATLGDESRIVTAFSRETPQKVYVQHRLQENGEELWNLLQKGAYIYVCGDAKNMARDVNQTFVNFAIEFGGQTEEKAHDYVKNLRNSGRYQEDVWS

SEQ ID NO: 247 - DNA, 2106 bp, Thamnostylum piriforme
  source 1..2106: mol_type = genomic DNA
  misc_feature 1..2106: note = 14HC_CPR_5
atggctcaatcttctccagctttgttggattctttggatattgtttttttgggtactattggtttgggtactattgcttggtttgctagaagacaaattgctgaaagaatttttggttctaaagatgatgctaataaaaatgttggtaatggtaatgctccaactgctccaaaaagagaaagaaattttgttaaagttatgcaagaacaaggtagaaaagttatttttttttatggttctcaaactggtactgctgaagattatgcttctagattggctaaagaatgttctcaaaaatatggtgtttcttgtatgactgctgatattgaattgtatgatttgacttatttggatactgttccagaagattttttggttttttttattatggctacttatggtgaaggtgaaccaactgataatgctgttgatttttgggaacaattgactgaagaagaaccacaattttctgaaggtgatactttgggtaatttgagatatgttgtttttggtttgggtaataaaacttatgaacattataatgaagttgctagaagaatggataaattgttgactaaattgggtgctaaaagaattggtgaaagaggtgaaggtgatgatgatgcttctttggaagaagattttttggcttggcaagattctatgtggccagctttttgtgatgctttgggtgttgatgaatctaatggttcttctggtccaagacaagctatgtatgctgttgaagaattggaaggtcaagaagctgtttatttgggtgaattgggtgaaaaaccaaaagaaggtgttaaagttgtttatgatgctaaaagaccatataatgctccattggtttctcaagatttgtttaaaaatactgatagacattgtttgcatattgatattgatgtttctgattctaatttgtcttatcaaactggtgatcatattgctatttggccaactaattctgatgatgaagttgctagattggcttctttgttgggtttgactgataaattggatacttctgttatggttaaagctattgattctactgcttctaaacaatatccatttccagttccagctacttatagatctatttttagacattatttggatatttgtgctccagcttctagacaaactttgatgtctttggttgaatatgctccaactgaagcatctaaagaagcattgagattgttgtctaaagataaagatgaatatagattgaaagttggtgaagctgttagaaatttgggtgaagttttggaattggctgctggtgctgatgctagaccaggtttgttttctactgttccatttgatttgattgttgaatctgtttctagattgcaaccaagatattattctatttcttcttctgctaaagaatctccaaaagttattgctgttactgctgttactttgacttataatccagatccaactccagaaagaactgtttatggtgttaatactaattatttgtggcaaattcatgctgctaaacataatgttaatgatggtcaaagatacccaacttatgatttggctggtccaagaaatgctttgcaaggtgctaaagttccagttcatattagaagatctcaatttaaattgccaagaaatccaactgttccagttattatggttggtccaggtactggtgttgctccatttagaggttttgttagagaaagagctgctcaaaaaactgatggtaaaccagttggtccaactttgttgttttttggttgtagaaattctcaacaagattttttgtataaagatgaatggccagaattgtttgctactttgggtgatgaatctagaattgttactgctttttctagagaaactccacaaaaagtttatgttcaacatagattgcaagaaaatggtgaagaattgtggaatttgttgcaaaaaggtgcttatatttatgtttgtggtgatgctaaaaatatggctagagatgttaatcaaacttttgttaattttgctattgaatttggtggtcaaactgaagaaaaagctcatgattatgttaaaaatttgagaaattctggtagataccaagaagatgtttggtcttag

SEQ ID NO: 248 - protein, 798 aa, Saccharomyces cerevisiae and Homo sapiens
  REGION 1..798: note = 14HC_CPR_6
MPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNLEDFICNAEAGALSNLRYNMFGLGNSTYKFFNGAAKKAEKHLSAAGAIRLGKLGEADDGAGTTDEDYMAWKDSILEVLKDELHLDEQEAKFTSQFQYTVLNEITDSMSLGEPSAHYLPSHQLNRNADGIQLGPFDLSQPYIAPIVKSRELFSSNDRNCIHSEFDLSGSNIKYSTGDHLAVWPSNPLEKVEQFLSIFNLDPETIFDLKPLDPTVKVPFPTPTTIGAAIKHYLEITGPVSRQLFSSLIQFAPNADVKEKLTLLSKDKDQFAVEITSKYFNIADALKYLSDGAKWDTVPMQFLVESVPQMTPRYYSISSSSLSEKQTVHVTSIVENFPNPELPDAPPVVGVTTNLLRNIQLAQNNVNIAETNLPVHYDLNGPRKLFANYKLPVHVRRSNFRLPSNPSTPVIMIGPGTGVAPFRGFIRERVAFLESQKKGGNNVSLGKHILFYGSRNTDDFLYQDEWPEYAKKLDGSFEMVVAHSRLPNTKKVYVQDKLKDYEDQVFEMINNGAFIYVCGDAKGMAKGVSTALVGILSRGKSITTDEATELIKMLKTSGRYQEDVWRRAMAEQSDEAVKYYTLEEIQKHNHSKSTWLILHHKVYDLTKFLEEHPGGEEVLREQAGGDATENFEDVGHSTDAREMSKTFIIGELHPDDRPKLNKPPETLITTID

SEQ ID NO: 249 - DNA, 2397 bp, Saccharomyces cerevisiae and Homo sapiens
  source 1..2397: mol_type = genomic DNA
  misc_feature 1..2397: note = 14HC_CPR_6
atgccgtttggaatagacaacaccgacttcactgtcctggcggggctagtgcttgccgtgctactgtacgtaaagagaaactccatcaaggaactgctgatgtccgatgacggagatatcacagctgtcagctcgggcaacagagacattgctcaggtggtgaccgaaaacaacaagaactacttggtgttgtatgcgtcgcagactgggactgccgaggattacgccaaaaagttttccaaggagctggtggccaagttcaacctaaacgtgatgtgcgcagatgttgagaactacgactttgagtcgctaaacgatgtgcccgtcatagtctcgatttttatctctacatatggtgaaggagacttccccgacggggcggtcaaccttgaagactttatttgtaatgcggaagcgggtgcactatcgaacctgaggtataatatgtttggtctgggaaattctacttataaattctttaatggtgccgccaagaaggccgagaagcatctctccgccgcgggcgctatcagactaggcaagctcggtgaagctgatgatggtgcaggaactacagacgaagattacatggcctggaaggactccatcctggaggttttgaaagacgaactgcatttggacgaacaggaagccaagttcacctctcaattccagtacactgtgttgaacgaaatcactgactccatgtcgcttggtgaaccctctgctcactatttgccctcgcatcagttgaaccgcaacgcagacggcatccaattgggtcccttcgatttgtctcaaccgtatattgcacccatcgtgaaatctcgcgaactgttctcttccaatgaccgtaattgcatccactctgaatttgacttgtccggctctaacatcaagtactccactggtgaccatcttgctgtttggccttccaacccattggaaaaggtcgaacagttcttatccatattcaacctggaccctgaaaccatttttgacttgaagcccctggatcccaccgtcaaagtgcccttcccaacgccaactactattggcgctgctattaaacactatttggaaattacaggacctgtctccagacaattgttttcatctttgattcagttcgcccccaacgctgacgtcaaggaaaaattgactctgctttcgaaagacaaggaccaattcgccgtcgagataacctccaaatatttcaacatcgcagatgctctgaaatatttgtctgatggcgccaaatgggacaccgtacccatgcaattcttggtcgaatcagttccccaaatgactcctcgttactactctatctcttcctcttctctgtctgaaaagcaaaccgtccatgtcacctccattgtggaaaactttcctaacccagaattgcctgatgctcctccagttgttggtgttacgactaacttgttaagaaacattcaattggctcaaaacaatgttaacattgccgaaactaacctacctgttcactacgatttaaatggcccacgtaaacttttcgccaattacaaattgcccgtccacgttcgtcgttctaacttcagattgccttccaacccttccaccccagttatcatgatcggtccaggtaccggtgttgccccattccgtgggtttatcagagagcgtgtcgcgttcctcgaatcacaaaagaagggcggtaacaacgtttcgctaggtaagcatatactgttttatggatcccgtaacactgatgatttcttgtaccaggacgaatggccagaatacgccaaaaaattggatggttcgttcgaaatggtcgtggcccattccaggttgccaaacaccaaaaaagtttatgttcaagataaattaaaggattacgaagaccaagtatttgaaatgattaacaacggtgcatttatctacgtctgtggtgatgcaaagggtatggccaagggtgtgtcaaccgcattggttggcatcttatcccgtggtaaatccattaccactgatgaagcaacagagctaatcaagatgctcaagacttcaggtagataccaagaagatgtctggagaagagctatggccgaacaatctgatgaagctgttaagtactacaccttggaagaaatccaaaagcacaaccattctaagtctacctggttgatcttgcatcacaaggtttacgatttgaccaagttcttagaagaacatccaggtggtgaagaagtcttgagagaacaagctggtggtgatgctactgaaaattttgaagatgttggtcattccaccgatgccagagaaatgtctaagactttcattatcggtgaattgcacccagatgatagaccaaaattgaacaaaccaccagaaaccttgattaccaccatcgattaa

SEQ ID NO: 250 - protein, 787 aa, Homo sapiens; identical to SEQ ID NO: 238
  REGION 1..787: note = 14HC_CPR_3

SEQ ID NO: 251 - DNA, 2364 bp, Homo sapiens; identical to SEQ ID NO: 239
  source 1..2364: mol_type = genomic DNA
  misc_feature 1..2364: note = 14HC_CPR_3

SEQ ID NO: 252 - protein, 692 aa, Arabidopsis thaliana [sequence omitted]

SEQ ID NO: 253 - DNA, 2079 bp, Arabidopsis thaliana; identical to SEQ ID NO: 241
  source 1..2079: mol_type = genomic DNA
  misc_feature 1..2079: note = 14HC_CPR_4

SEQ ID NO: 254 - protein, 676 aa, Alosa sapidissima
  REGION 1..676: note = 14HC_CPR_24
MADVQSEAPTQPDLMEADEPLLSNLDIFLFSLIAGLVIYWFTFRKKPEPIPEFKKLDMPAPSTRETSFIEKMKKTGRNIVVFYGSQTGTAEEFSNRLSKDAQRYGMKGMAADPEEYDLSELARLSEIENSLAIFCMATYGEGDPTDNAQDFYDWLQEGDADLDGLNFAVFALGNKTYEHYNAMGKYVDKRLEELGGKRVFDLGMGDDDGNLEEDFVSWREQFWPAMCEHFGVEATGEESSIRQYELKVHDDLNMNKVYTGELGRLKSFESQKPPFDSKNPFLAPVTVNRKLNKGGDRHLMHLELDITGSKIRYESGDHVAVYPINDTTIVTKLGDILGVDLDTVISLNNLDEESNKKHPFPCPTTYRTALTHYLDITHPPRTNVLYELAQYATDPKDQENMRKMASSSSEGKALYQSFVLDPSRNILAILEDMPSLKPPADHLCELLPRLQARYYSIASSSKVHPNSIHVCCVVVEYKTSTGRTNKGVATNWLKSKLPTDNGHKATVPMYVRKSQFRLPFKATNPVIMIGPGTGIAPFIGFVQERGWLKEQGKEVGETVLYYGCRHKNEDYLYQEELEEFEKTGVLTKLNVAFSRDQDQKVYVQHLIKNNKEEIWNQVHSQSAHIYICGDARNMARDVQAAFYDIAEELGGMTHAQATDYVKKLMTKGRYSQDVWS

SEQ ID NO: 255 - DNA, 2028 bp, Alosa sapidissima
  source 1..2028: mol_type = genomic DNA
  misc_feature 1..2028: note = 14HC_CPR_24
atggcggatgtacaaagcgaggctcccactcaacccgacctaatggaagcggatgaacctctgttatcaaacttggatatttttctttttagcctgattgctgggttagttatctattggttcacgtttagaaagaagccggaaccaattcccgagttcaagaagctagacatgcctgctcccagtacacgtgaaacatccttcatagaaaagatgaagaagacagggagaaatattgtcgttttctatggctcacaaacaggaactgcagaggagttttccaatagactttccaaggatgcacaacgttacggtatgaagggtatggctgcagacccagaagagtatgacctttcagaattggcgaggttgagcgaaatagaaaattcattagccattttttgtatggccacatacggcgaaggtgatccaacggacaacgctcaggatttttacgattggttgcaagagggggacgctgatctagacggtcttaactttgctgttttcgcactaggtaacaagacgtacgaacattacaatgcaatgggtaagtatgtcgataagcgtttagaggaactaggcggcaaaagagtcttcgaccttggtatgggggacgacgatgggaatctagaagaggacttcgtaagttggagagaacagttctggcctgccatgtgcgagcattttggcgtagaggcaacaggcgaagagtcttctatacgtcagtacgaacttaaagtacacgacgacttaaatatgaataaggtatacacaggagagttgggtaggttaaagtcctttgagtcacaaaagccgccattcgactccaaaaatccctttttagctccagtaacagtaaatcgtaagctgaacaagggcggtgatcgtcatttaatgcatctggaattagacattacagggagcaagattagatatgagtcaggcgatcatgtggcggtttatcctatcaatgacactactatcgtaacgaaactgggtgatattttaggggtagacctggacactgttataagtttaaataatcttgatgaggaatccaataagaagcatccgttcccgtgtccaacaacctacagaacagctttaacacattatttagacataacacatccacccaggactaacgttctatatgaactagctcagtacgcgacggaccccaaggatcaggagaacatgagaaagatggcaagctcatcttccgagggaaaagcattgtatcagtcatttgtgctagacccctcaaggaacatactagccatattagaagacatgccatcactaaaaccgcccgccgaccatttgtgtgaattgctaccgaggctgcaagcacgttattatagcatcgcttcaagctccaaggtacatcccaacagtattcacgtgtgctgtgtcgtcgtagagtacaagacctccacgggcagaaccaacaagggggtagctacaaactggcttaagtctaaactaccgacggataatggccacaaagcaacggtgcccatgtacgttcgtaagagtcaattcaggttgcctttcaaagctactaaccctgtcataatgattggaccagggacgggaattgcaccgttcattgggttcgtccaggaaagaggctggttgaaggaacagggcaaagaagttggagaaacagttctgtattatggatgtaggcataaaaatgaggactacctataccaagaagagttggaggaattcgaaaagacgggcgtcctaacaaagttgaacgtggccttctctagggatcaggaccagaaggtatatgtacaacacttaataaagaacaataaagaggaaatatggaaccaagtgcactctcagtcagcacacatctatatctgcggggacgcgaggaacatggcgagagatgtgcaagctgcgttctatgacatcgctgaagaattgggggggatgacgcacgctcaagccacggattatgtaaagaagctgatgaccaaaggcaggtacagtcaggacgtgtggtca

SEQ ID NO: 256 - protein, 712 aa, Circinella minor
  REGION 1..712: note = 14HC_CPR_28
MAYSKKTTSSLLAITPLDIFLGGILGLGTIIWISRQFRSSSFKYDNSNSKPDNQKTAVNTGPERNFVKLMKQQNRQVIFFYGSQTGTAEDYASRLAKECSQKYGVSAMTADIELYDMTYLDAVPSDNLVVFVMATYGEGEPTDNAVEFWELFNGEEPEFSESTTLENLKYIVFGLGNKTYEHYNAVARVLDTRLSELGAKRIGERGEGDDDASLEEDFLAWQENMWPHFCNALGVDESNAQSGPRQVMFGVEELDAVDKTSLFLGELGEWKNPEQLHQTVVYDAKHPYQAPISLSRDLFNESDDRHCLHLEIDLSKSNLTYQTGDHIAIWPTNNEVEVERFGSILGLSDKLDTAIMVEALDSAASKKHPFPVPTTYRALFRHYLDICAPASRQTLMSLVEYAPTESSKETLRQLATDKDVYRIKVGEAMRNLAEVLELCQDDDEGTATKMSSGFFSTVPFDLIVESLSRLQPRYYSISSSASTSPKSVAVTAVTLAYQPTKEEDRTVYGVNTNYLWRIHAPEDNSIPAYDLNGPRDTLKNQKIPVHIRRSQFKLPRNTKLPVIMVGPGTGVAPFRGFVQERVYQKQQGKEVGPTVLFYGCRRSDQDFLYKDEWPQLFKDLGEESRIITAFSRETEQKVYVQHRLKEHGEKMWEYMEQGALLYVCGDAKNMARDVNQTFVSFAIEFGGKTEQQAQDYVKNLRTSGRYQEDVWS

SEQ ID NO: 257 - DNA, 2136 bp, Circinella minor
  source 1..2136: mol_type = genomic DNA
  misc_feature 1..2136: note = 14HC_CPR_28
atggcgtatagtaagaagacgacaagtagcctgcttgctataacaccccttgatatctttctgggcggaatcttagggctgggcactattatatggattagcaggcaatttagatcaagctcttttaaatatgacaacagtaatagtaaacccgataatcaaaaaacggccgtcaatacggggccagaaaggaatttcgtcaaattgatgaaacaacagaacagacaggtaatttttttctatggcagccagaccggaactgcggaggattacgcgagcaggttggcgaaggaatgcagccaaaaatatggcgtgtctgcaatgactgccgatatagaactttacgatatgacctacttggacgccgtaccgtcagataacctagtggttttcgttatggctacctacggagaaggggaaccgacggacaatgcggtcgagttttgggagctttttaacggggaagagccggagttttcagagagcacaacgctagaaaatctgaaatacatcgtgttcggattagggaacaaaacgtacgagcattacaacgccgtagccagggtgctggacacgagactaagcgagctaggggccaagagaatcggtgagcgtggcgagggtgacgatgacgcaagtctagaggaagattttttagcgtggcaggaaaatatgtggccgcatttctgtaatgcgctgggagtagacgaaagtaatgcgcagtccgggcctcgtcaagttatgttcggcgtagaagaacttgacgccgtagacaagacgagtctttttttaggtgaactaggagagtggaaaaaccctgagcagttacaccagacagtagtgtatgatgcgaagcatccttatcaggcgcctattagcttatctagggacctttttaacgagagcgacgatcgtcactgcctgcacttagagattgacttgagtaaatccaacctaacctaccaaaccggggatcacatcgcgatatggcctacgaataacgaggttgaggtggaaagattcggaagtatactgggtctaagtgacaagctggatactgccatcatggttgaggcgctggacagtgcagctagcaagaaacatccattccccgtacctacaacttatagggctctgtttagacattatctggacatttgcgcacccgcaagccgtcagacattgatgagcctagtagagtatgcacccacggagtctagcaaggagacactgagacagcttgccacggataaggatgtgtataggataaaggtaggggaggctatgagaaacttggccgaggttctggaactttgccaggatgatgacgaaggaacggctacaaagatgtcctcaggtttcttctccaccgtaccatttgatcttattgtcgagagtctaagcaggcttcaacccaggtactactctatctccagtagcgcgtccacctcaccgaaatctgtggctgtaactgcggtaacgttggcctaccaacctaccaaagaagaggataggacggtatatggtgtaaacacaaattatttatggagaatccacgcccccgaggacaactccataccagcctacgatttaaatggtccaagggacacgctaaagaaccagaagatccccgtgcacatcagacgttcacagttcaagctgccgcgtaacacgaagttgccggtgataatggtcggtcctgggaccggcgttgcccctttccgtggcttcgttcaggagcgtgtgtatcaaaagcagcagggtaaagaggttgggccgaccgttcttttttacggatgcaggcgtagtgatcaagattttttgtacaaagacgaatggccacaactgttcaaagaccttggcgaagagagcaggattataaccgccttttcaagagagactgagcaaaaagtttatgtccaacacagattgaaggagcatggggagaaaatgtgggaatacatggagcaaggcgcattgctatacgtatgcggggacgccaagaatatggcccgtgatgttaaccagacgttcgttagcttcgccattgaatttggtggtaaaacggaacagcaggcccaggattatgtgaagaatctgcgtacaagtggaaggtaccaagaggacgtctggtcc

SEQ ID NO: 258 - protein, 713 aa, Rhizopus delemar
  REGION 1..713: note = 14HC_CPR_29
MTRNNSHHLLDTVDLILLGTIGLGTVAWFARHQIANRLFKSDSTNKSEVKDEAKTPKQERNFVKVMQQQGRRVIFFYGSQTGTAEDFASRLAKECTQKYGVSAMTADIEQYDLSYLDSVPEDSLVFFIMATYGEGEPTDNAVDFWDLLAEEVPEFSNDDGEGKPLQKLRYVAFGLGNKTYEHYNEVIRKVDNRLLSLGAKRIGERGEGDDDGTLEEDFLAWQEEMWPAFCEALGVDESNAHSGPRQAIFKIEELTAYDQAKVYLGEIGEWLKEGASIVYDAKRPYNAPITSKDIFKAGDRHCLHLEIDISNTNLSYQTGDHVAIWPTNNEVEVNRLAKLLGLQNKLDTVIHVQSLDPAASKKYPFPVPTTYRAVFRHYLDICSAVPRQVLMSLIEYAPTEASKEALRKLATDKDEYRVHVGDVTRNLGEVLQMLAESESLELDGAFSSVPFDLVIESISRLQPRYYSISSSSKENPKKIAVTAVTLQYTPEHGSPRTVYGVNTNYLWRVHEAVNNLTPNSVIPEYNLTGPRDSLFSQEDKVARIPVHVRRSQFKLPRNPTVPIIMIGPGTGVAPFRGFVRERALQKKENKPVGPTILFFGCRNRAEDFLYEEEWPELFEVLGGDSRIITAFSRETEKKVYVQHRLMENGQEMWNLLEKGAYVYVCGDAKNMARDVNQTFVRFAQQFGGMDENRSQDYVKNLRNTGRYQEDVWS

SEQ ID NO: 259 - DNA, 2139 bp, Rhizopus delemar
  source 1..2139: mol_type = genomic DNA
  misc_feature 1..2139: note = 14HC_CPR_29
atgacaagaaacaattcacaccatttgcttgacactgtagatctgattttgttgggcacaataggccttggtacagtcgcctggtttgctagacaccagattgctaataggcttttcaagagcgattccacgaataagtccgaagttaaagatgaagcgaagacacctaagcaagaacgtaactttgttaaggtaatgcagcaacaaggcaggagagtaatcttcttttatgggtcccagacgggtacagcggaggacttcgctagtcgtctagctaaagagtgtacccaaaagtatggtgtttctgctatgaccgcggacattgagcaatatgaccttagctaccttgactcagtgcccgaggatagcctagtgtttttcatcatggctacttatggcgaaggagagccaacggataacgctgtcgatttttgggacctacttgcggaagaggtacccgagtttagcaatgacgacggtgaagggaagccgttgcagaaattaaggtatgtagcgtttgggctaggcaataaaacgtatgagcattataatgaagtcatccgtaaagtcgacaatagactgctgtcacttggagcaaagaggattggtgagcgtggggagggggatgatgacggcacgttagaagaggactttcttgcctggcaagaggaaatgtggcccgctttttgtgaggcgttgggggtagacgaatcaaatgctcacagcgggccgcgtcaggccatcttcaagatagaagagcttacggcttatgatcaagccaaagtctacctgggagagataggggagtggctgaaggagggagcctctatagtttatgatgcaaagaggccatacaacgctcctattacctccaaagacatcttcaaagctggggatagacattgcttacatttggaaatagatattagtaatactaacctgtcataccagacgggagaccacgttgcgatttggcccaccaataacgaagttgaggtaaatcgtcttgcaaaactgctaggtctgcaaaataaattagatacggtaattcacgttcaatccctagatccggcagcttcaaaaaaatacccgttccccgtacccacgacctatagggcggtgttcaggcactatctggacatatgctccgcagtaccgaggcaagtacttatgagcttaatagaatacgcacccacggaagcctccaaggaagcgttgaggaagcttgcgacggataaagatgagtatcgtgtccatgtaggagatgttacacgtaatttgggtgaggttttacagatgctagctgagagcgagtccctagagctggatggcgcgttttcctcagtacccttcgacctagttattgaatcaatatcaagattgcaaccgaggtactacagtatctctagcagttcaaaggagaatcctaagaagatagcagtgacggcagtgaccttacaatatacaccggagcatggttctcctagaactgtttacggtgtaaacactaactatttatggcgtgttcacgaagccgttaacaatttaacgccgaactcagtcattcctgaatataatttgaccggaccgagggacagtctattctcacaggaggacaaagttgccaggatccctgtgcatgttcgtagatcccaatttaagcttcctagaaaccccacggttcctataattatgatagggcccggaactggcgttgctccctttagggggttcgtaagggaacgtgcgttgcaaaaaaaggaaaacaaaccggtaggtccaactatcttatttttcgggtgcaggaaccgtgccgaagactttctatatgaagaggaatggcccgaactatttgaggtgcttgggggggactctcgtattattaccgcatttagtcgtgagacagagaaaaaagtctatgtccaacacaggttgatggaaaacggacaagaaatgtggaacttattggagaaaggcgcgtatgtgtacgtttgcggggacgccaagaatatggcaagagacgtcaatcaaacattcgtgaggtttgcccagcaattcggagggatggatgagaacaggtcccaggactatgtcaagaatttacgtaatacagggcgttaccaggaagacgtttggtcc

SEQ ID NO: 260 - protein, 710 aa, Mucor ambiguus
  REGION 1..710: note = 14HC_CPR_30
MDPEPATALFDTTDLVLLTAIGLGTAAWFSRHQLAELFGKKKQKVVTAGTNTAQPKRETNFVKVMEQQGRRVIVFYGSQTGTAEGFAARIAKECSQRFGVSCMTADLELYDLTYLDQVPKDNLVVFVLATYGEGDPTDNAMEFWDLVTEEEPTFSESSEDRPLTNLNYVMFGLGNKTYDQYNEVSRRINKCLLKLGAHLVGEAGEGDDDASMEEDYLAWKEKMWPAFCETLGVDESSARSGPRQPVYMVEELPEYEKNEVYFGELAEKAKNNAKIIYDAKRPYIATMTSRELFHGADRHCMHIDVDISGTNLSYNTGDHLAIWPTNNEIEVNRLASVLGLTDKLDTVVMVKAIDPMAPKKHPFPVPTTYRSIFRYYIEICAPISRQVLNSFVEYAPSEEAKANLTRLATDKDEYRLQVGDACRNLGEVLQLVTGADTRPGLFANVPFDLIVETVARMQPRFYSISSSAKESPKAISATVVTLAYQPLPTPHRTVYGVNTNFLYSIQMQNESTVADGQEHPPYDLAGPRQAYLGSDGYCHKIPVHVRHSQFKLPRNPSVPVIMVGPGTGVAPFRGFVRERVVEKRNGKPVGTTLLFYGSRNQAQDFLYADEWPELFETLGGESRIVTAFSRDQAQKIYVQHRLQEHGAQVWELLQKGGYVYVCGDAKSMARDVNQTFINCAIEYGGMTDDKAHEYVKGLRTTGRYQEDVWS

SEQ ID NO: 261 - DNA, 2130 bp, Mucor ambiguus
  source 1..2130: mol_type = genomic DNA
  misc_feature 1..2130: note = 14HC_CPR_30
atggaccccgagcccgcgacggctctttttgacacaacagacttagtattattaaccgctataggtctagggacagcagcgtggttcagcagacatcagttggcggagttgttcggcaaaaagaagcaaaaggttgtcactgcgggtacgaacactgcccagccgaagagagagactaatttcgtgaaggtaatggaacaacaaggccgtagggtcatcgtattttatggtagtcagactggaactgcggagggtttcgcggctagaattgcaaaagagtgttctcagcgttttggcgtgtcttgtatgactgcagacctagagttatacgacctaacgtatcttgaccaggtgcccaaagataatctggtcgtctttgtattagccacgtatggcgaaggggacccaacggacaatgccatggagttctgggaccttgtaacagaggaagagccaacctttagtgagtcttctgaggatcgtccattaaccaacctgaactacgttatgttcggtctaggtaataagacatacgaccagtataatgaggtttctagaagaatcaataaatgtcttctgaaacttggtgcccatcttgtgggtgaggctggagaaggcgacgacgatgcttccatggaagaagactatctagcttggaaggagaagatgtggcctgctttttgtgaaacgttgggggtggacgagagttctgcacgttccggacctcgtcaacccgtatatatggttgaagagttaccggaatatgagaaaaatgaggtttattttggagaattggccgagaaggcaaaaaataatgcaaaaattatttacgacgccaagcgtccttatattgctaccatgaccagtcgtgagctgtttcacggtgctgatcgtcactgtatgcacatagacgtggatattagcgggaccaacctaagttataatacgggtgatcacctagcaatctggcccaccaataacgagatcgaagttaataggctagcgtctgtgttaggtcttaccgataagttggatactgtcgttatggttaaggcgatcgaccccatggcgccgaaaaaacatccttttcccgtaccgacgacttatagatcaatctttagatactatattgagatatgtgctcccatatccaggcaagtcttgaacagctttgtggagtacgcaccttcagaagaagctaaagctaacctgactcgtctggccaccgacaaggatgaatacagactgcaggtcggagatgcctgcaggaatttgggagaggtcctgcagcttgttacaggtgcggataccaggcccggattattcgcaaacgttccgtttgatctaatagtcgagacggtggccaggatgcagccgcgtttttacagtatcagcagtagtgctaaggaatctcccaaggcgattagcgcaaccgttgttacgctggcctatcaaccattgcctacccctcatagaaccgtgtatggcgttaatactaactttttgtattccatacagatgcagaacgagtctactgtcgccgacggacaggaacacccgccctatgacctagccgggccgagacaggcatacctgggatccgatgggtactgtcataagattccagtacacgttaggcacagccaatttaagctgccgagaaacccgtccgtccccgtcatcatggtcggaccaggaaccggggtagcgcctttcagaggtttcgtccgtgagagggtcgttgagaagaggaacgggaaaccagtaggaacaacactacttttctatgggtcacgtaaccaggcgcaagatttcttatacgccgacgagtggcccgaactatttgagacgctgggcggagaatcaagaattgtaactgcgttctcaagagaccaagctcaaaaaatatacgtacagcatagactacaggagcatggcgctcaagtctgggaattgcttcaaaagggaggctatgtatatgtctgtggagatgctaagtccatggcgcgtgatgtcaaccaaactttcataaattgtgctatcgagtatggagggatgaccgacgacaaggcgcatgagtatgtgaagggtctacgtaccactggcagataccaggaagatgtctggtca

SEQ ID NO: 262 - protein, 694 aa, Thamnostylum piriforme
  REGION 1..694: note = 14HC_CPR_31
MAKNTTLNKQVVLLISALGLSAVAYMASRYFFGNSASTPEQSKKPIQPEEKEDESNFVQTMKKQNRKVVIFYGSQTGTAEDCAQRLGKQCKKQFGVPALVLDIEQCNMSYLDQIPEDCVAIFVMATYGEGEPTDNATDFWEMLEAHPEFSQSDNLKNLRYFIFGLGNSSYTYYNWVSKTVDKKLTELGATRLGKLGLGDDEKSLEDDFEAWQEGMWPIFGEAVETDAASNISGGHEPTYHVVELTEHDGHVYSGELGDKSQALYSGKKPYPAPITTRDLLNGSDRHCLHLEVDISGSGMSYTTGDHIAIWPTNSEDEVLSLARAIGLENKLDTVICVTAADETSAKQSPFPQPTTYRAMLRHYLDICQMPSRQTLEMLVPFAPSPEAAETLEKLAKDKDEHRRVVLGPVRNLAAVLSYASSGAAYKIPSDVLLECLGRLQPRYYSISSSALESPNSVSVTAVTLKFNPEPTPERTVFGVNTNYLWAVHTALNDDDEAIQVETEYSIGGPNGQYFDEKLKTARLPVHIRQSNFKLPADTTKPVIMIGPGTGVAPFRAFVRERAYQKKHLGKSIGPTILYFGCRRSNEDFLYQEEWPELFNELGGSSRLITAFSRETDRKVYVQDKLNENGPETWDLINNKGAYVYVCGDAKRMAKDVQSALVSFAKQYGSYDDAGAADYISKLRDAGRYQEDVWA

SEQ ID NO: 263 - DNA, 2085 bp, Thamnostylum piriforme
  source 1..2085: mol_type = genomic DNA
  misc_feature 1..2085: note = 14HC_CPR_31
atggcaaaaaatactacattaaacaagcaagtggtgcttctcatcagtgccctgggcctgagcgctgttgcctacatggccagccgatacttttttggaaacagcgcaagcacccctgaacaatcgaaaaagcccattcaaccagaagaaaaagaagacgaatcaaactttgtgcaaaccatgaagaagcagaatcgtaaagtagtgatcttttatggttcgcaaacaggcacggctgaagattgtgcgcagcgacttggcaagcagtgcaaaaagcaattcggcgttcctgctttggttcttgatatcgaacaatgcaacatgtcgtacttggatcagattcccgaggactgtgttgcgatatttgtcatggcaacttatggcgaaggtgaacccactgacaacgccactgacttttgggaaatgcttgaagcccatcctgaattttcccaaagcgacaacctcaagaatctccgatacttcatcttcggacttggcaacagctcctatacctattacaactgggtctccaaaactgtcgacaagaaactcactgaattaggcgcgacacgacttggcaaacttggcttgggcgacgacgaaaagtcgctcgaagacgactttgaagcatggcaggaaggcatgtggcctatttttggagaagctgtggaaacggacgccgccagcaatatcagcggcggacacgagcccacataccatgtggtggagctaaccgaacatgacggccacgtgtattctggtgaattgggagacaaatcgcaagctctctatagcggcaagaagccttacccagcacctattaccacgcgcgatttgttgaacggatcggatcgccactgtctacacctggaagtcgatatcagtggatcaggcatgagctataccactggcgatcatattgccatctggcccacaaacagcgaagacgaggtcttgagcttggcacgcgccattggtctcgaaaacaagctggataccgtcatttgcgttactgctgcggatgagacgtccgccaagcaatcaccgttccctcagccaacaacataccgtgccatgctgcgacactacttggatatttgccaaatgccatcacggcagacactggagatgctcgtaccttttgcaccgtcgcccgaagctgcagagactctagaaaagctggcaaaggacaaggatgagcatcgtcgagtggttctgggaccagttcgtaacttggctgctgtgctgagttatgcgtcgtcgggcgctgcatacaagatcccatccgatgtgctgcttgagtgccttggtcgactacagccccgatattactcgatttcctcctctgccttggagagcccaaacagtgtcagtgtcactgccgttacactcaaattcaaccccgaaccaacgcctgagcgcactgtctttggcgtcaataccaactacctgtgggccgtccataccgcgctcaacgatgacgacgaggcaatccaagttgaaaccgagtattccatcggtggacctaacggtcaatactttgacgagaagctcaaaactgcaagattgcctgttcacatccgccaatcaaacttcaagctgccagcagacacaactaagccagtgatcatgattggccccggcaccggtgtcgctcctttccgagccttcgttcgcgagcgtgcctatcaaaagaagcaccttggcaaatccatcggtccaactattctgtactttggctgccgacgttccaacgaggactttttgtatcaggaagagtggccagagctctttaacgaattgggcggctcctctcgattgatcactgcattctcccgggaaacggatcgaaaagtatacgtccaagacaagctcaacgagaacggcccagagacttgggatttgattaataacaagggcgcctatgtctatgtttgtggcgacgctaagcgtatggccaaagacgtccagtccgcacttgttagttttgcaaagcagtatggcagctatgacgacgctggtgcagctgactacatcagcaagttaagagatgccggacgatatcaagaagacgtctgggcataa

SEQ ID NO: 264 - protein, 710 aa, Cunninghamella elegans
  REGION 1..710: note = 14HC_CPR_32
MAQQTPAVIDTLDLILLGSIGLGTIAWFTRRQLSERLFGTGQSNATPKPTTPQAPKRERNFVKVMEQQGRKVIFFYGSQTGTAEDFASRLAKQCSQKYGVSCMTADIEMYDLSYLDTLSEDSLVCFVMATYGEGEPTDNAVDFWEQFITDESPVFSEGGETLENLRYLMFGLGNKTYEHYNAVARILDKKLTGLGAKRIGERGEGDDDGSLEEDFLAWQESMWPTFCDALGVDENNAQQGPRQASYSVDELKEEYKNDDVYFGELGTKSKDSSRVVYDAKRPYNAPITTRELFNSSERHCLHVDIDISGTNLSYQTGDHVAMWPTNNEDEVLRLANILGLQDKLDNVISVKAIDPAAPKQHPFPVPTTYRAIFRHYIDICAPASRQSLMSFVEFAPTDSAKDLLKLLATDKDEYRLKVGEAVRNLGEVLELVSGNDKDTQPGSFTSVPFDLIVETIPRLQPRYYSISSSSKENSSIISATCVTLAYQPDPTPDRTVYGVNTNFLYRIHTQSSDNDIQGLPKYDLAGPRKAFLNEQGQSHKLPIHIRRSQFKLPRNTSCPVIMIGPGTGVAPFRGFVRERALQKKEGKSVGPTVLFFGNRHSEHDFLYSDEWPELFNTLGDDSKLITAFSRETEHKVYVQHRLEENGKDIWQLLEKGAYIYVCGDARNMARDVNQTFVNLAMEYGEKTEQKALDYVKSLRNTGRYQEDVWS

SEQ ID NO: 265 - DNA, 2133 bp, Cunninghamella elegans
  source 1..2133: mol_type = genomic DNA
  misc_feature 1..2133: note = 14HC_CPR_32
atggcacagcaaacaccagccgttatcgacactttggatcttattttgttaggctccatcggcctgggaacaatcgcctggtttacacgtagacagttatctgagagattatttgggactgggcaatcaaatgcaacgccaaaacccacgactccacaggctccgaagcgtgagaggaactttgttaaggttatggaacagcaagggagaaaggttatctttttctatggatctcagacgggaactgccgaagacttcgcgtctagattagctaagcagtgcagccagaagtatggcgtgtcttgcatgacagctgatatagaaatgtacgatcttagttacctagataccttgtccgaggacagtttggtatgttttgtcatggcaacttacggagaaggtgagcctacggataatgcagtagacttctgggaacaatttatcacagacgaatcacccgtgttttcagagggaggagaaaccctagagaatttgcgttatctaatgtttggtttggggaataaaacttacgagcactacaatgccgtagcccgtattcttgataagaaacttaccggtttgggggctaagcgtattggagaaaggggggaaggggatgacgatggctcactagaagaggactttttggcgtggcaagagtctatgtggcccacattttgtgacgcattgggagttgatgaaaataacgcccaacaggggcccagacaggcaagctacagtgtcgatgagttaaaagaggagtataaaaacgatgacgtctacttcggtgagctgggaaccaagtcaaaggattcctccagagttgtttatgacgcgaagcgtccgtacaacgccccaataacgacgagagagctatttaattctagcgaaagacattgcctgcacgttgacatagacatctcaggaactaatctgtcctatcaaacgggtgatcacgtggccatgtggccaaccaacaatgaagatgaagtgctgagattagcgaacatcttgggactgcaagataaactggacaatgttataagcgttaaggccattgatccggcagctccaaagcaacacccctttcctgttcctaccacttatagggcaatttttcgtcactatattgatatttgcgcgcccgcttcaaggcaaagtctgatgagtttcgtagaattcgctcccacagattccgccaaagacttactaaagcttctagctacggataaagatgagtataggctgaaggtgggtgaggccgtgaggaaccttggcgaggtccttgagttggtttccggcaatgacaaagacacacagccgggaagctttaccagtgtaccgtttgatctgatagttgagacgatacctcgtcttcaacccagatattactcaatcagttcctcttccaaggaaaattcatctattatctccgcgacttgcgttacgctagcttaccaaccggaccccaccccggatagaacggtatatggtgtgaacactaatttcctttaccgtattcacacccagagtagtgacaacgatatccaagggctgcccaagtacgacctagcggggccgagaaaagcctttctgaacgaacaaggccaatcacacaaactacctattcacatccgtcgttcccagtttaaattgccaagaaatacttcctgtccggtgatcatgattggtcccgggactggcgtagcgcctttccgtgggtttgtcagggaaagggcgcttcagaaaaaagaaggtaagtccgttgggccaactgtcctgtttttcggtaatagacatagtgaacacgacttcctatattctgatgagtggccagagctgtttaatacattgggggacgattccaaacttatcactgcctttagtagggaaacagaacacaaagtctacgtacaacaccgtctagaagaaaatgggaaagacatctggcaattgttagaaaagggtgcgtatatttacgtgtgtggagacgccagaaacatggcccgtgacgtgaaccaaacgttcgttaatctagcgatggagtatggggagaaaactgagcagaaggcactagactatgtgaagtctcttcgtaatactggacgttatcaggaggatgtttggtcataa

SEQ ID NO: 266 - DNA, 6887 bp, Saccharomyces cerevisiae
  source 1..6887: mol_type = genomic DNA
  misc_feature 1..6887: note = pA36 (containing 14HC_P450_5)
tgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgattcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgacagagatcaaggagcacatctacagataccgtcactatatcggggttgcggctgctgtcgctttggtgtgccaacaagtgtattacaggatctttcgtatccctaagaacctgaggcacatacctgccattccgtacggtcaacaactaaaggcattgaggtccgatgaggctcttaccagcaggacgaagcgtttagtattccctttgctatctaagtgtaatggggtataccttaatagaatgccatttaagtggacaatttatgtcgcggatccagaaatcgcgagggcgattctttttaaacccgaattcggccacaagaccaggagtgtgacagattccttggataagaactcaagcctatttgaatttgttggggacgataatatagccatagttaatggccatgaatggaaagagcaacgtaagataatgaacccagcattccacagggctacgcccgtagggatgttcggcagtttaatgcctaaagtttttaggcttgtggaagaacagcctacactacccgccctagatttaatgcaaaagttaacgttagacgcacttgggaagtcagtattcggctttgaattcggcgcactagatgaccccgacagtgtgtgggttaagacttatagacaagtgtttgactcttttaccgacgttttctctctagtgtttccccgtttagatccaatctaccgttatttttctgcgaaacacagagaacagtacaacgctgtttacaaactaattgatctgctggatggaatggcagacaagaaaaggagcatgttgcaagatgcctccaactccgatatcaaagatgttccagaccacgagaaagatttactgcaacttatgttggaggctgaactgcgtggcgaaggctcatggacaaaacgtgaattgcgtcataacatggccatattttttgtggcaggtcaggacacaacgagtcatgctttgactttttgcctttatttacttgcgaagaatcaagatatccagaagaaggcgagagaagaaatcttaaatgtttttggggatgaacctaaagacgtgtttcccacacttgaagattgcaaaaaactaaattatcttgacatggtgatcaaggaatccatgagaatttaccctcctgctaacgatgtcctagccagggatgtcaatgaagatttaaacgtcaagggtgtcttcattcccaagggatctatggtatcagtggacattcatgccctacaccatagaccagacctgtggcatgagcccgataagttcaatcccgatcgtttcctgccgggtggggagcatgacagtcacgtcggcgtaacatacgcccccttctcatccggctccagacagtgcatcgcgttaaaattcgctaccatgcagcaaagggtagtcctgtccatgttgctgagaaagtacgaatgggaattgccaaaggattccaaacataaggactccatccagttccagattcccttcaacatcgctcctaaggatctagagcttacctttcataaacgttactagacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgccctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcc

SEQ ID NO: 267 - DNA, 7547 bp, Saccharomyces cerevisiae
  source 1..7547: mol_type = genomic DNA
  misc_feature 1..7547: note = pA48 (containing 14HC_P450_5)
aacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgccctcgccgcagttaattaaagtcagtgagcgaggaagcgcgtaactataacggtcctaaggtagcgaatcctgatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgattcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggctcaatctccaccagcattggatactttggatatcgttttcttgggtactatcggtttgggtacaattgcttggtttgctagaaggcaaattgctgaaagattattcggttccacctcttcctctgatgctaaatctaatggtcatgctactccagctccaccaaaaagagaaagaaacttcgttaagatcatgcaagagcaaggtagaaaggtcattttcttctacggttctcaaactggtactgctgaagattacgcttctagattggctaaagaatgctctcaaaagtacggtgttaactgtatgactgctgatttggagttgtacgacttgtcttacttggatactgttccagaagattgcctggtttttttcgttatggctacttatggtgaaggtgaacctactgataatgctgttgatttctgggatgtcttgtctgaagaagaaccacaattttctgaagccgaaggtgataagccattgcaaaatttgagatacttggttttcggcttgggtaacaagacttacgaacattataacgctgttgccagaaacgttgacaagagattggaagttttgggtgctcatagaattcatgaacgtggtgagggtgatgatgatggttctttggaagaagattttttggcctggcaagaaaacatgtggccagctttttgtgaagctttgggtgttgatgaatctaacgctcattctggtccaagacaagctacttattctgttgaagaattggtcgatgtcaacatggatgatgtttacttgggtgaattggccgaaaaacctaaagaaggtgctagagttatctacgatgctaagaggccttttaatgctccaattgccatttctcaagacttgttcactaacaccgatagacattgcttgcacatggaaatcgatatctccgattccaacttgtcataccaaaccggtgatcatattgctatttggccaactaactccgaaaacgaagttgctagactggcttctattttgggtttagctgataagttggataccgccatcaatgttaaggctattgatccagctgcttccaaaaagtacccatttccttgtccagctacttacagagctattttcagacattacttggacatttgcgctgccgtttctagacaatctttgatggcttttgttgaatacgctccaaccgaagaatccaaagaaagattgagacaattggccaaggataaggatgagtacagattgactgttggtgaagctgttagaaatttgggtgaagtattggaaatcgttgctggtaatgatgttaagccaggtttcttttcttccgttccattcgatttggttgtcgaatctatttccagattgcaacctaggtactactccatttcttcatctgcaaaagagtccccaaaaaagatcgctgttactgctgttacattgtcctatcaaccagatccaactccacaaagaactgtttatggtgtcaacactaattacttgtggcgtattcataccgcctctaagcaacaatctactgaagctgatttgccaacctatgatttgtcaggtccaagaaatgctttacatggtactaagttgccagttcacgttagaagatctcaattcaagttgccaagaaacccaaccgttccagttattatggttggtccaggtactggtgttgctccttttagaggttttgttagagaaagagccttgcagaaatctgaaggtaaaccagttggtccaactttgttgtttttcggttgtagaaactccgaacaggacttcttgtacaaagatgaatggcctgctttgtttgacaccttaggtgaatcctctagaattattaccgccttctcaagagaaaccgctcaaaaagtttacgtccagcatagattgcaagagaacggtcaagaagtttggggtttgttgcaaaggggtgcttatatctatgtttgtggtgatgcaaagaacatggccagagatgttcaacaaactttcgttaacttcggtatcgaattcggtggtttgtctgatgataaggctcatgattttgtcaagaacttgagaaacaccggtagataccaagaagatgtttggtcttgaacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcg